(19) United States
(12) Patent Application Publication
(10) Pub. No.: US 2001/0034023 A1
(43) Pub. Date: Oct. 25, 2001

(54) Gene sequence variations with utility in determining the treatment of disease, in genes relating to drug processing

(76) Inventors: Vincent P. Stanton JR., Belmont, MA (US); Martin Zillmann, Shrewsbury, MA (US)

Correspondence Address: EDWARD O. KRUESSER, BROBECK PHLEGER & HARRISON, 12390 EL CAMINO REAL, SAN DIEGO, CA, 92130, US

(21) Appl. No.: 09/733,000

(22) Filed: Dec. 7, 2000

Related U.S. Application Data
(63) Continuation-in-part of application No. 09/710,467, filed on Nov. 8, 2000.
Application No. 09/710,467 is a continuation-in-part of application No. 09/696,482, filed on Oct. 24, 2000.
(60) Provisional application No. 60/131,334, filed on Apr. 26, 1999.
(60) Provisional application No. 60/139,440, filed on Jun. 15, 1999.

(30) Foreign Application Priority Data
Jan. 20, 2000 (WO) PCT/US00/01392

(51) Int. Cl. C12Q 1/68; G06F 19/00

(52) U.S. Cl. 435/6; 702/20

(57) ABSTRACT

Methods for identifying and utilizing variances in genes relating to efficacy and safety of medical therapy and other aspects of medical therapy are described, including methods for selecting an effective treatment.

### RELATED APPLICATIONS

This application is a continuation-in-part of Stanton et al., U.S. application Ser. No. 09/710,467, filed Nov. 8, 2000 entitled GENE SEQUENCE VARIATIONS WITH UTILITY IN DETERMINING THE TREATMENT OF DISEASE, IN GENES RELATING TO DRUG PROCESSING, which is a continuation-in-part of Stanton et al., U.S. application Ser. No. 09/696,482, filed Oct. 24, 2000 entitled GENE SEQUENCE VARIATIONS WITH UTILITY IN DETERMINING THE TREATMENT OF DISEASE, IN GENES RELATING TO DRUG PROCESSING, which is a continuation-in-part of U.S. application Ser. No. not yet assigned, Attorney Docket No. 030586.0009CIP4, filed Oct. 6, 2000 entitled GENE SEQUENCE VARIATIONS WITH UTILITY IN DETERMINING THE TREATMENT OF DISEASE, IN GENES RELATING TO DRUG PROCESSING, which is a continuation-in-part of Stanton et al., U.S. application Ser. No. 09/639,474, filed Aug. 15, 2000 GENE SEQUENCE VARIATIONS WITH UTILITY IN DETERMINING THE TREATMENT OF DISEASE, IN GENES RELATING TO DRUG PROCESSING, which is a continuation in part of Stanton et al., U.S. application Ser. No. 09/590,783, filed Jun. 8, 2000 GENE SEQUENCE VARIATIONS WITH UTILITY IN DETERMINING THE TREATMENT OF DISEASE, IN GENES RELATING TO DRUG PROCESSING, which is a continuation-in-part of Stanton, U.S. application Ser. No. 09/501,955, filed Feb. 10, 2000, which is a continuation-in-part of Stanton, International Application Ser. No. PCT/US00/01392, filed Jan. 20, 2000, Stanton, U.S. application Ser. No. 09/427,835, filed Oct. 26, 1999, and Stanton et al., U.S. application Ser. No. 09/300,747, filed Apr. 26, 1999, and claims the benefit of U.S. Provisional Patent Application, Stanton & Adams, Ser. No. 60/131,334, filed Apr. 26, 1999, and U.S. Provisional Patent Application, Stanton, Ser. No. 60/139,440, filed Jun. 15, 1999, which are hereby incorporated by reference in their entireties, including drawings and tables.

### BACKGROUND OF THE INVENTION

This application concerns the field of mammalian therapeutics and the selection of therapeutic regimens utilizing host genetic information, including gene sequence variances within the human genome in human populations.

The information provided below is not admitted to be prior art to the present invention, but is provided solely to assist the understanding of the reader.

Many drugs or other treatments are known to have highly variable safety and efficacy in different individuals. A consequence of such variability is that a given drug or other treatment may be effective in one individual, and ineffective or not well-tolerated in another individual. Thus, administration of such a drug to an individual in whom the drug would be ineffective would result in wasted cost and time during which the patient's condition may significantly worsen. Also, administration of a drug to an individual in whom the drug would not be tolerated could result in a direct worsening of the patient's condition and could even result in the patient's death.

For some drugs, over 90% of the measurable intersubject variation in selected pharmacokinetic parameters has been shown to be heritable. For a limited number of drugs, DNA sequence variances have been identified in specific genes that are involved in drug action or metabolism, and these variances have been shown to account for the variable efficacy or safety of the drugs in different individuals. As the sequence of the human genome is completed, and as additional human gene sequence variances are identified, the power of genetic methods for predicting drug response will further increase. This application concerns methods for identifying and exploiting gene sequence variances that account for interpatient variation in drug response, particularly interpatient variation attributable to pharmacokinetic factors and interpatient variation in drug tolerability or toxicity.

The efficacy of a drug is a function of both pharmacodynamic effects and pharmacokinetic effects, or bioavailability. In the present invention, interpatient variability in drug safety, tolerability and efficacy are discussed in terms of the genetic determinants of interpatient variation in absorption, distribution, metabolism, and excretion, i.e. pharmacokinetic parameters.

Adverse drug reactions are a principal cause of the low success rate of drug development programs (less than one in four compounds that enters human clinical testing is ultimately approved for use by the U.S. Food and Drug Administration (FDA)). Adverse drug reactions can be categorized as 1) mechanism based reactions and 2) idiosyncratic, “unpredictable” effects apparently unrelated to the primary pharmacologic action of the compound. Although some side effects appear shortly after administration, in some instances side effects appear only after a latent period. Adverse drug reactions can also be categorized into reversible and irreversible effects. The methods of this invention are useful for identifying the genetic basis of both mechanism based and ‘idiosyncratic’ toxic effects, whether reversible or not. Methods for identifying the genetic sources of interpatient variation in efficacy and mechanism based toxicity may be initially directed to analysis of genes affecting pharmacokinetic parameters, while the genetic causes of idiosyncratic adverse drug reactions are more likely to be attributable to genes affecting variation in pharmacodynamic responses or immunological responsiveness.

Absorption is the first pharmacokinetic parameter to consider when determining the causes of intersubject variation in drug response. The relevant genes depend on the route of administration of the compound being evaluated. For orally administered drugs the major steps in absorption may occur during exposure to salivary enzymes in the mouth, exposure to the acidic environment of the stomach, exposure to pancreatic digestive enzymes and bile in the small intestine, exposure to enteric bacteria and exposure to cell surface proteins throughout the gastrointestinal tract. For example, uptake of a drug that is absorbed across the gastrointestinal tract by facilitated transport may vary on account of allelic variation in the gene encoding the transporter protein. Many drugs are lipophilic (a property which promotes passive movement across biological membranes). Variation in levels of such drugs may depend, for example, on the enterohepatic circulation of the drug, which may be affected by genetic variation in liver canalicular transporters, or intestinal transporters; alternatively renal reabsorbtion mechanisms may vary among patients as a consequence of gene sequence variances. If a compound is delivered parenterally then absorption is not an issue, however transcutaneous administration of a compound may be subject to genetically determined variation in skin absorptive properties.

Once a drug or candidate therapeutic intervention is absorbed, injected or otherwise enters the bloodstream it is distributed to various biological compartments via the blood. The drug may exist free in the blood, or, more commonly, may be bound with varying degrees of affinity to plasma proteins. One classic source of interpatient variation in drug response is attributable to amino acid polymorphisms in serum albumin, which affect the binding affinity of drugs such as warfarin. Consequent interpatient variation in levels of free warfarin have a significant effect on the degree of anticoagulation. From the blood a compound diffuses into and is retained in interstitial and cellular fluids of different organs to different degrees. Interpatient variation in the levels of a drug in different anatomical compartments may be attributable to variation in the genetically encoded chemical environment of those tissues (cell surface proteins, matrix proteins, cytoplasmic proteins and other factors)

Once absorbed by the gastrointestinal tract, compounds encounter detoxifying and metabolizing enzymes in the tissues of the gastrointestinal system. Many of these enzymes are known to be polymorphic in man and account for well studied variation in pharmacokinetic parameters of many drugs. Subsequently compounds enter the hepatic portal circulation in a process commonly known as first pass. The compounds then encounter a vast array of xenobiotic detoxifying mechanisms in the liver, including enzymes that are expressed solely or at high levels only in liver. These enzymes include the cytochrome P450s, glucuronlytransferases, sulfotransferases, acetyltransferases, methyltransferases, the glutathione conjugating system, flavine monooxygenases, and other enzymes known in the art. Polymorphisms have been detected in all of these metabolizing systems, however the genetic factors responsible for intersubject variation have only been partially identified, and in some cases not yet identified at all. Biotransformation reactions in the liver often have the effect of converting lipophilic compounds into hydrophilic molecules that are then more readily excreted. Variation in these conjugation reactions may affect half-life and other pharmacokinetic parameters. It is important to note that metabolic transformation of a compound not infrequently gives rise to a second or additional compounds that have biological activity greater than, less than, or different from that of the parent compound. Metabolic transformation may also be responsible for producing toxic metabolites.

Biotransformation reactions can be divided into two phases. Phase I are oxidation-reduction reactions and phase II are conjugation reactions. The enzymes involved in both of these phases are located predominantly in the liver, however biotransformation can also occur in the kidney, gastrointestinal tract, skin, lung, and other organs. Phase I reactions occur predominantly in the endoplasmic reticulum, while phase II reactions occur predominantly in the cytosol. Both types of reactions can occur in the mitochondria, nuclear envelope, or plasma membrane. One skilled in the art can, for some compounds, make reasonable predictions concerning likely metabolic systems given the structure of the compound. Experimental means of assessing relevant biotransformation systems are also described.

Drug-induced disease or toxicity presents a unique series of challenges to drug developers, as these reactions are often not predictable from preclinical studies and may not be detected in early clinical trials involving small numbers of subjects. When such effects are detected in later stages of clinical development they often result in termination of a drug development program because, until now, there have been no effective tools to seek the determinants of such reactions. When a drug is approved despite some toxicity, its clinical use is frequently severely constrained by the possible occurrence of adverse reactions in even a small group of patients. The likelihood of such a compound becoming first line therapy is small (unless there are no competing products). Thus, clinical trials that lead to detection of genetic causes of adverse events and subsequently to the creation of genetic tests to identify and screen out patients susceptible to such events have the potential to (i) enable approval of compounds for genetically circumscribed populations or (ii) enable repositioning of approved compounds for broader clinical use.

Similarly, many compounds are not approved due to unimpressive efficacy. The identification of genetic determinants of pharmacokinetic variation may lead to identification of a genetically defined population in whom a significant response is occurring. Approval of a compound for this population, defined by a genetic diagnostic test, may be the only means of getting regulatory approval for a drug. As healthcare becomes increasingly costly, the ability to allocate healthcare resources effectively becomes increasingly urgent. The use of genetic tests to develop and rationally administer medicines represents a powerful tool for accomplishing more cost effective medical care.

### SUMMARY OF THE INVENTION

The present invention is concerned generally with the field of pharmacology, specifically pharmacokinetics and toxicology, and more specifically with identifying and predicting inter-patient differences in response to drugs in order to achieve superior efficacy and safety in selected patient populations. It is further concerned with the genetic basis of inter-patient variation in response to therapy, including drug therapy, and with methods for determining and exploiting such differences to improve medical outcomes. Specifically, this invention describes the identification of genes and gene sequence variances useful in the field of therapeutics for optimizing efficacy and safety of drug therapy by allowing prediction of pharmacokinetic and/or toxicologic behavior of specific drugs in specific patients. Relevant pharmacokinetic processes include absorption, distribution, metabolism and excretion. Relevant toxicological processes include both dose related and idiosyncratic adverse reactions to drugs, including, for example, hepatotoxicity, blood dyscrasias and immunological reactions. The invention also describes methods for establishing diagnostic tests useful in (i) the development of, (ii) obtaining regulatory approval for and (iii) safe and efficacious clinical use of pharmaceutical products. These variances may be useful either during the drug development process or in guiding the optimal use of already approved compounds. DNA sequence variances in candidate genes (i.e. genes that may plausibly affect the action of a drug) are tested in clinical trials, leading to the establishment of diagnostic tests useful for improving the development of new pharmaceutical products and/or the more effective use of existing pharmaceutical products. Methods for identifying genetic variances and determining their utility in the selection of optimal therapy for specific patients are also described. In general, the invention relates to methods for identifying and dealing effectively with the genetic sources of interpatient variation in drug response, including both variable efficacy as determined by pharmacokinetic variability and variable toxicity as determined by pharmacokinetic factors or by other genetic factors (e.g. factors responsible for idiosyncratic drug response).

The inventors have determined that the identification of gene sequence variances in genes that may be involved in drug action are useful for determining whether genetic variances account for variable drug efficacy and safety and for determining whether a given drug or other therapy may be safe and effective in an individual patient. Provided in this invention are identifications of genes and sequence variances which can be useful in connection with predicting differences in response to treatment and selection of appropriate treatment of a disease or condition. A target gene and variances have utility in pharmacogenetic association studies and diagnostic tests to improve the use of certain drugs or other therapies including, but not limited to, the drug classes and specific drugs identified in the 1999 Physicians' Desk Reference (53rd edition), Medical Economics Data, 1998, or the 1995 United States Pharmacopeia XXIII National Formulary XVIII, Interpharm Press, 1994, or other sources as described below.

The terms “disease” or “condition” are commonly recognized in the art and designate the presence of signs and/or symptoms in an individual or patient that are generally recognized as abnormal. Diseases or conditions may be diagnosed and categorized based on pathological changes. Signs may include any objective evidence of a disease such as changes that are evident by physical examination of a patient or the results of diagnostic tests which may include, among others, laboratory tests to determine the presence of DNA sequence variances or variant forms of certain genes in a patient. Symptoms are subjective evidence of disease or a patients condition, i.e. the patients perception of an abnormal condition that differs from normal function, sensation, or appearance, which may include, without limitations, physical disabilities, morbidity, pain, and other changes from the normal condition experienced by an individual. Various diseases or conditions include, but are not limited to; those categorized in standard textbooks of medicine including, without limitation, textbooks of nutrition, allopathic, homeopathic, and osteopathic medicine. In certain aspects of this invention, the disease or condition is selected from the group consisting of the types of diseases listed in standard texts such as Harrison's Principles of Internal Medicine (14th Ed) by Anthony S. Fauci, Eugene Braunwald, Kurt J. Isselbacher, et al. (Editors), McGraw Hill, 1997, or Robbins Pathologic Basis of Disease (6th edition) by Ramzi S. Cotran, Vinay Kumar, Tucker Collins & Stanley L. Robbins, W B Saunders Co., 1998, or the Diagnostic and Statistical Manual of Mental Disorders: DSM-IV (4^{th} edition), American Psychiatric Press, 1994, or other texts described below.

In connection with the methods of this invention, unless otherwise indicated, the term “suffering from a disease or condition” means that a person is either presently subject to the signs and symptoms, or is more likely to develop such signs and symptoms than a normal person in the population. Thus, for example, a person suffering from a condition can include a developing fetus, a person subject to a treatment or environmental condition which enhances the likelihood of developing the signs or symptoms of a condition, or a person who is being given or will be given a treatment which increase the likelihood of the person developing a particular condition. For example, tardive dyskinesia is associated with long-term use of anti-psychotics; dyskinesias, paranoid ideation, psychotic episodes and depression have been associated with use of L-dopa in Parkinson's disease; and dizziness, diplopia, ataxia, sedation, impaired mentation, weight gain, and other undesired effects have been described for various anticonvulsant therapies, alopecia and bone marrow suppression are associated with cancer chemotherapeutic regimens, and immunosuppression is associated with agents to limit graft rejection following transplantation. Thus, methods of the present invention which relate to treatments of patients (e.g., methods for selecting a treatment, selecting a patient for a treatment, and methods of treating a disease or condition in a patient) can include primary treatments directed to a presently active disease or condition, secondary treatments which are intended to cause a biological effect relevant to a primary treatment, and prophylactic treatments intended to delay, reduce, or prevent the development of a disease or condition, as well as treatments intended to cause the development of a condition different from that which would have been likely to develop in the absence of the treatment.

The term “therapy” refers to a process that is intended to produce a beneficial change in the condition of a mammal, e.g., a human, often referred to as a patient. A beneficial change can, for example, include one or more of: restoration of function, reduction of symptoms, limitation or retardation of progression of a disease, disorder, or condition or prevention, limitation or retardation of deterioration of a patient's condition, disease or disorder. Such therapy can involve, for example, nutritional modifications, administration of radiation, administration of a drug, behavioral modifications, and combinations of these, among others.

The term “drug” as used herein refers to a chemical entity or biological product, or combination of chemical entities or biological products, administered to a person to treat or prevent or control a disease or condition. The chemical entity or biological product is preferably, but not necessarily a low molecular weight compound, but may also be a larger compound, for example, an oligomer of nucleic acids, amino acids, or carbohydrates including without limitation proteins, oligonucleotides, ribozymes, DNAzymes, glycoproteins, lipoproteins, and modifications and combinations thereof. A biological product is preferably a monoclonal or polyclonal antibody or fragment thereof such as a variable chain fragment; cells; or an agent or product arising from recombinant technology, such as, without limitation, a recombinant protein, recombinant vaccine, or DNA construct developed for therapeutic, e.g., human therapeutic, use. The term “drug” may include, without limitation, compounds that are approved for sale as pharmaceutical products by government regulatory agencies (e.g., U.S. Food and Drug Administration (USFDA or FDA), European Medicines Evaluation Agency (EMEA), and a world regulatory body governing the International Conference of Harmonization (ICH) rules and guidelines), compounds that do not require approval by government regulatory agencies, food additives or supplements including compounds commonly characterized as vitamins, natural products, and completely or incompletely characterized mixtures of chemical entities including natural compounds or purified or partially purified natural products. The term “drug” as used herein is synonymous with the terms “medicine”, “pharmaceutical product”, or “product”. Most preferably the drug is approved by a government agency for treatment of a specific disease or condition.

The term “candidate therapeutic intervention” refers to a drug, agent or compound that is under investigation, either in laboratory or human clinical testing for a specific disease, disorder, or condition.

A “low molecular weight compound” has a molecular weight <5,000 Da, more preferably <2500 Da, still more preferably <1000 Da, and most preferably <700 Da.

Those familiar with drug use in medical practice will recognize that regulatory approval for drug use is commonly limited to approved indications, such as to those patients afflicted with a disease or condition for which the drug has been shown to be likely to produce a beneficial effect in a controlled clinical trial. Unfortunately, it has generally not been possible with current knowledge to predict which patients will have a beneficial response, with the exception of certain diseases such as bacterial infections where suitable laboratory methods have been developed. Likewise, it has generally not been possible to determine in advance whether a drug will be safe in a given patient. Regulatory approval for the use of most drugs is limited to the treatment of selected diseases and conditions. The descriptions of approved drug usage, including the suggested diagnostic studies or monitoring studies, and the allowable parameters of such studies, are commonly described in the “label” or “insert” which is distributed with the drug. Such labels or inserts are preferably required by government agencies as a condition for marketing the drug and are listed in common references such as the Physicians Desk Reference (PDR). These and other limitations or considerations on the use of a drug are also found in medical journals, publications such as pharmacology, pharmacy or medical textbooks including, without limitation, textbooks of nutrition, allopathic, homeopathic, and osteopathic medicine.

Many widely used drugs are effective in a minority of patients receiving the drug, particularly when one controls for the placebo effect. For example, the PDR shows that about 45% of patients receiving Cognex (tacrine hydrochloride) for Alzheimer's disease show no change or minimal worsening of their disease, as do about 68% of controls (including about 5% of controls who were much worse). About 58% of Alzheimer's patients receiving Cognex were minimally improved, compared to about 33% of controls, while about 2% of patients receiving Cognex were much improved compared to about 1% of controls. Thus a tiny fraction of patients had a significant benefit. Response to many cancer chemotherapy drugs is even worse. For example, 5-fluorouracil is standard therapy for advanced colorectal cancer, but only about 20-40% of patients have an objective response to the drug, and, of these, only 1-5% of patients have a complete response (complete tumor disappearance; the remaining patients have only partial tumor shrinkage). Conversely, up to 20-30% of patients receiving 5-FU suffer serious gastrointestinal or hematopoietic toxicity, depending on the regimen.

Thus, in a first aspect, the invention provides a method for selecting a treatment for a patient suffering from a disease or condition by determining whether or not a gene or genes in cells of the patient (in some cases including both normal and disease cells, such as cancer cells) contain at least one sequence variance which is indicative of the effectiveness of the treatment of the disease or condition. The gene or genes are preferably specified herein, in Table 1, 3, or 4. Preferably the at least one variance includes a plurality of variances which may provide a haplotype or haplotypes. Preferably the joint presence of the plurality of variances is indicative of the potential effectiveness or safety of the treatment in a patient having such plurality of variances. The plurality of variances may each be indicative of the potential effectiveness of the treatment, and the effects of the individual variances may be independent or additive, or the plurality of variances may be indicative of the potential effectiveness if at least 2, 3, 4, or more appear jointly. The plurality of variances may also be combinations of these relationships. The plurality of variances may include variances from one, two, three or more gene loci.

In preferred embodiments of aspects of the invention involving genes relating to pharmacokinetic parameters that affect efficacy and safety, e.g. drug-induced disease or drug-induced, disorder, or dysfunction or other drug-induced pathophysiologic disease, or protection or sensitivity to toxic compounds, the gene product is involved in a function as described in the Background of the Invention or otherwise described herein.

In some cases, the selection of a method of treatment, i.e., a therapeutic regimen, may incorporate selection of one or more from a plurality of medical therapies. Thus, the selection may be the selection of a method or methods which is/are more effective or less effective than certain other therapeutic regimens (with either having varying safety parameters). Likewise or in combination with the preceding selection, the selection may be the selection of a method or methods, which is safer than certain other methods of treatment in the patient.

The selection may involve either positive selection or negative selection or both, meaning that the selection can involve a choice that a particular method would be an appropriate method to use and/or a choice that a particular method would be an inappropriate method to use. Thus, in certain embodiments, the presence of the at least one variance is indicative that the treatment will be effective or otherwise beneficial (or more likely to be beneficial) in the patient. Stating that the treatment will be effective means that the probability of beneficial therapeutic effect is greater than in a person not having the appropriate presence or absence of particular variances. In other embodiments, the presence of the at least one variance is indicative that the treatment will be ineffective or contra-indicated for the patient. For example, a treatment may be contra-indicated if the treatment results, or is more likely to result, in undesirable side effects, or an excessive level of undesirable side effects. A determination of what constitutes excessive side-effects will vary, for example, depending on the disease or condition being treated, the availability of alternatives, the expected or experienced efficacy of the treatment, and the tolerance of the patient. As for an effective treatment, this means that it is more likely that desired effect will result from the treatment administration in a patient with a particular variance or variances than in a patient who has a different variance or variances. Also in preferred embodiments, the presence of the at least one variance is indicative that the treatment is both effective and unlikely to result in undesirable effects or outcomes, or vice versa (is likely to have undesirable side effects but unlikely to produce desired therapeutic effects).

In reference to response to a treatment, the term “tolerance” refers to the ability of a patient to accept a treatment, based, e.g., on deleterious effects and/or effects on lifestyle. Frequently, the term principally concerns the patients perceived magnitude of deleterious effects such as nausea, weakness, dizziness, and diarrhea, among others. Such experienced effects can, for example, be due to general or cell-specific toxicity, activity on non-target cells, cross-reactivity on non-target cellular constituents (non-mechanism based), and/or side effects of activity on the target cellular substituents (mechanism based), or the cause of toxicity may not be understood. In any of these circumstances one may identify an association between the undesirable effects and variances in specific genes.

Adverse responses to drugs constitute a major medical problem, as shown in two recent meta-analyses (Lazarou, J. et al, Incidence of adverse drug reactions in hospitalized patients: a meta-analysis of prospective studies, JAMA 279:1200-1205, 1998; Bonn, Adverse drug reactions remain a major cause of death, Lancet 351:1183, 1998). An estimated 2.2 million hospitalized patients in the United Stated had serious adverse drug reactions in 1994, with an estimated 106,000 deaths (Lazarou et al.). To the extent that some of these adverse events are due to genetically encoded biochemical diversity among patients in pathways that effect drug action, the identification of variances that are predictive of such effects will allow for more effective and safer drug use.

In embodiments of this invention, the variance or variant form or forms of a gene is/are associated with a specific response to a drug. The frequency of a specific variance or variant form of the gene may correspond to the frequency of an efficacious response to administration of a drug. Alternatively, the frequency of a specific variance or variant form of the gene may correspond to the frequency of an adverse event resulting from administration of a drug. Alternatively the frequency of a specific variance or variant form of a gene may not correspond closely with the frequency of a beneficial or adverse response, yet the variance may still be useful for identifying a patient subset with high response or toxicity incidence because the variance may account for only a fraction of the patients with high response or toxicity. In such a case the preferred course of action is identification of a second or third or additional variances that permit identification of the patient groups not usefully identified by the first variance. Preferably, the drug will be effective in more than 20% of individuals with one or more specific variances or variant forms of the gene, more preferably in 40% and most preferably in >60%. In other embodiments, the drug will be toxic or create clinically unacceptable side effects in more than 10% of individuals with one or more variances or variant forms of the gene, more preferably in >30%, more preferably in >50%, and most preferably in >70% or in more than 90%.

Also in other embodiments, the method of selecting a treatment includes eliminating a treatment, where the presence or absence of the at least one variance is indicative that the treatment will be ineffective or contra-indicated, e.g., would result in excessive weight gain. In other preferred embodiments, in cases in which undesirable side-effects may occur or are expected to occur from a particular therapeutic treatment, the selection of a method of treatment can include identifying both a first and second treatment, where the first treatment is effective to treat the disease or condition, and the second treatment reduces a deleterious effect of the first treatment.

The phrase “eliminating a treatment” refers to removing a possible treatment from consideration, e.g., for use with a particular patient based on the presence or absence of a particular variance(s) in one or more genes in cells of that patient, or to stopping the administration of a treatment which was in the course of administration.

Usually, the treatment will involve the administration of a compound preferentially active or safe in patients with a form or forms of a gene, where the gene is one identified herein. The administration may involve a combination of compounds. Thus, in preferred embodiments, the method involves identifying such an active compound or combination of compounds, where the compound is less active or is less safe or both when administered to a patient having a different form of the gene.

Also in preferred embodiments, the method of selecting a treatment involves selecting a method of administration of a compound, combination of compounds, or pharmaceutical composition, for example, selecting a suitable dosage level and/or frequency of administration, and/or mode of administration of a compound. The method of administration can be selected to provide better, preferably maximum therapeutic benefit. In this context, “maximum” refers to an approximate local maximum based on the parameters being considered, not an absolute maximum.

Also in this context, a “suitable dosage level” refers to a dosage level which provides a therapeutically reasonable balance between pharmacological effectiveness and deleterious effects. Often this dosage level is related to the peak or average serum levels resulting from administration of a drug at the particular dosage level.

Similarly, a “frequency of administration” refers to how often in a specified time period a treatment is administered, e.g., once, twice, or three times per day, every other day, once per week, etc. For a drug or drugs, the frequency of administration is generally selected to achieve a pharmacologically effective average or peak serum level without excessive deleterious effects (and preferably while still being able to have reasonable patient compliance for self-administered drugs). Thus, it is desirable to maintain the serum level of the drug within a therapeutic window of concentrations for the greatest percentage of time possible without such deleterious effects as would cause a prudent physician to reduce the frequency of administration for a particular dosage level.

A particular gene or genes can be relevant to the treatment of more than one disease or condition, for example, the gene or genes can have a role in the initiation, development, course, treatment, treatment outcomes, or health-related quality of life outcomes of a number of different diseases, disorders, or conditions. Thus, in preferred embodiments, the disease or condition or treatment of the disease or condition is any which involves a gene from the gene list described herein as Tables 1, 3, and 4.

Determining the presence of a particular variance or plurality of variances in a particular gene in a patient can be performed in a variety of ways. In preferred embodiments, the detection of the presence or absence of at least one variance involves amplifying a segment of nucleic acid including at least one of the at least one variances. Preferably a segment of nucleic acid to be amplified is 500 nucleotides or less in length, more preferably 100 nucleotides or less, and most preferably 45 nucleotides or less. Also, preferably the amplified segment or segments includes a plurality of variances, or a plurality of segments of a gene or of a plurality of genes.

In another aspect determining the presence of a set of variances in a specific gene related to treatment of pharmacokinetic parameters associated efficacy or safety, e.g. drug-induced disease, disorder, dysfunction, or other toxicity-related gene or genes listed in Tables 1, 3 and 4 may entail a haplotyping test that requires allele specific amplification of a large DNA segment of no greater than 25,000 nucleotides, preferably no greater than 10,000 nucleotides and most preferably no greater than 5,000 nucleotides. Alternatively one allele may be enriched by methods other than amplification prior to determining genotypes at specific variant positions on the enriched allele as a way of determining haplotypes. Preferably the determination of the presence or absence of a haplotype involves determining the sequence of the variant site or sites by methods such as chain terminating DNA sequencing or minisequencing, or by oligonucleotide hybridization or by mass spectrometry.

The term “genotype” in the context of this invention refers to the alleles present in DNA from a subject or patient, where an allele can be defined by the particular nucleotide(s) present in a nucleic acid sequence at a particular site(s). Often a genotype is the nucleotide(s) present at a single polymorphic site known to vary in the human population.

In preferred embodiments, the detection of the presence or absence of the at least one variance involves contacting a nucleic acid sequence corresponding to one of the genes identified above or a product of such a gene with a probe. The probe is able to distinguish a particular form of the gene or gene product or the presence or a particular variance or variances, e.g., by differential binding or hybridization. Thus, exemplary probes include nucleic acid hybridization probes, peptide nucleic acid probes, nucleotide-containing probes which also contain at least one nucleotide analog, and antibodies, e.g., monoclonal antibodies, and other probes as discussed herein. Those skilled in the art are familiar with the preparation of probes with particular specificities. Those skilled in the art will recognize that a variety of variables can be adjusted to optimize the discrimination between two variant forms of a gene, including changes in salt concentration, temperature, pH and addition of various compounds that affect the differential affinity of GC vs. AT base pairs, such as tetramethyl ammonium chloride. (See Current Protocols in Molecular Biology by F. M. Ausubel, R. Brent, R. E. Kngston, D. D. Moore, J. D. Seidman, K. Struhl, and V. B. Chanda (editors, John Wiley & Sons.)

In other preferred embodiments, determining the presence or absence of the at least one variance involves sequencing at least one nucleic acid sample. The sequencing involves sequencing of a portion or portions of a gene and/or portions of a plurality of genes which includes at least one variance site, and may include a plurality of such sites. Preferably, the portion is 500 nucleotides or less in length, more preferably 200 or 100 nucleotides or less, and most preferably 45 nucleotides or less in length. Such sequencing can be carried out by various methods recognized by those skilled in the art, including use of dideoxy termination methods (e.g., using dye-labeled dideoxy nucleotides) and the use of mass spectrometric methods. In addition, mass spectrometric methods may be used to determine the nucleotide present at a variance site. In preferred embodiments in which a plurality of variances is determined, the plurality of variances can constitute a haplotype or collection of haplotypes. Preferably the methods for determining genotypes or haplotypes are designed to be sensitive to all the common genotypes or haplotypes present in the population being studied (for example, a clinical trial population).

The terms “variant form of a gene”, “form of a gene”, or “allele” refer to one specific form of a gene in a population, the specific form differing from other forms of the same gene in the sequence of at least one, and frequently more than one, variant sites within the sequence of the gene. The sequences at these variant sites that differ between different alleles of the gene are termed “gene sequence variances” or “variances” or “variants”. The term “alternative form” refers to an allele that can be distinguished from other alleles by having distinct variances at least one, and frequently more than one, variant sites within the gene sequence. Other terms known in the art to be equivalent include mutation and polymorphism, although mutation is often used to refer to an allele associated with a deleterious phenotype. In preferred aspects of this invention, the variances are selected from the group consisting of the variances listed in the variance tables herein or in a patent or patent application referenced and incorporated by reference in this disclosure. In the methods utilizing variance presence or absence, reference to the presence of a variance or variances means particular variances, i.e., particular nucleotides at particular polymorphic sites, rather than just the presence of any variance in the gene.

Variances occur in the human genome at approximately one in every 500-1,000 bases within the human genome when two alleles are compared. When multiple alleles from unrelated individuals are compared the density of variant sites increases as different individuals, when compared to a reference sequence, will often have sequence variances at different sites. At most variant sites there are only two alternative nucleotides involving the substitution of one base for another or the insertion/deletion of one or more nucleotides. Within a gene there may be several variant sites. Variant forms of the gene or alternative alleles can be distinguished by the presence of alternative variances at a single variant site, or a combination of several different variances at different sites (haplotypes).

It is estimated that there are 3,300,000,000 bases in the sequence of a single haploid human genome. All human cells except germ cells are normally diploid. Each gene in the genome may span 100-10,000,000 bases of DNA sequence or 100-20,000 bases of mRNA. It is estimated that there are between 60,000 and 120,000 genes in the human genome. The “identification” of genetic variances or variant forms of a gene involves the discovery of variances that are present in a population. The identification of variances is required for development of a diagnostic test to determine whether a patient has a variant form of a gene that is known to be associated with a disease, condition, or predisposition or with the efficacy or safety of the drug. Identification of previously undiscovered genetic variances is distinct from the process of “determining” the status of known variances by a diagnostic test (often referred to as genotyping). The present invention provides exemplary variances in genes listed in the gene tables, as well as methods for discovering additional variances in those genes and a comprehensive written description of such additional possible variances. Also described are methods for DNA diagnostic tests to determine the DNA sequence at a particular variant site or sites.

The process of “identifying” or discovering new variances involves comparing the sequence of at least two alleles of a gene, more preferably at least 10 alleles and most preferably at least 50 alleles (keeping in mind that each somatic cell has two alleles. The analysis of large numbers of individuals to discover variances in the gene sequence between individuals in a population will result in detection of a greater fraction of all the variances in the population. Preferably the process of identifying reveals whether there is a variance within the gene; more preferably identifying reveals the location of the variance within the gene; more preferably identifying provides knowledge of the sequence of the nucleic acid sequence of the variance, and most preferably identifying provides knowledge of the combination of different variances that comprise specific variant forms of the gene (referred to as alleles). In identifying new variances it is often useful to screen different population groups based on racial, ethnic, gender, and/or geographic origin because particular variances may differ in frequency between such groups. It may also be useful to screen DNA from individuals with a particular disease or condition of interest because they may have a higher frequency of certain variances than the general population.

The process of genotyping involves using diagnostic tests for specific variances that have already been identified. It will be apparent that such diagnostic tests can only be performed after variances and variant forms of the gene have been identified. Identification of new variances can be accomplished by a variety of methods, alone or in combination, including, for example, DNA sequencing, SSCP, heteroduplex analysis, denaturing gradient gel electrophoresis (DGGE), heteroduplex cleavage (either enzymatic as with T4 Endonuclease 7, or chemical as with osmium tetroxide and hydroxylamine), computational methods (described in “VARIANCE SCANNING METHOD FOR IDENTIFYING GENE SEQUENCE VARIANCES” filed Oct. 14, 1999, Ser. No. 09/419,705, and other methods described herein as well as others known to those skilled in the art. (See, for example: Cotton, R. G. H., Slowly but surely towards better scanning for mutations, Trends in Genetics 13(2): 43-6, 1997 or Current Protocols in Human Genetics by N. C. Dracoli, J. L. Haines, B. R. Korf, D. T. Moir, C. C. Morton, C. E. Seidman, D. R. Smith, and A. Boyle (editors), John Wiley & Sons.)

In the context of this invention, the term “analyzing a sequence” refers to determining at least some sequence information about the sequence,, e.g., determining the nucleotides present at a particular site or sites in the sequence, particularly sites that are known to vary in a population, or determining the base sequence of all of a portion of the particular sequence.

In the context of this invention, the term “haplotype” refers to a cis arrangement of two or more polymorphic nucleotides, i.e., variances, on a particular chromosome, e.g., in a particular gene. The haplotype preserves information about the phase of the polymorphic nucleotides—that is, which set of variances were inherited from one parent, and which from the other. A genotyping test does not provide information about phase. For example, an individual heterozygous at nucleotide 25 of a gene (both A and C are present) and also at nucleotide 100 (both G and T are present) could have haplotypes 25A-100G and 25C-100T, or alternatively 25A-100T and 25C-100G. Only a haplotyping test can discriminate these two cases definitively.

The terms “variances”, “variants” and “polymorphisms”, as used herein, may also refer to a set of variances, haplotypes or a mixture of the two. Further, the term variance, variant or polymorphism (singular), as used herein, also encompasses a haplotype. This usage is intended to minimize the need for cumbersome phrases such as: “. . . measure correlation between drug response and a variance, variances, haplotype, haplotypes or a combination of variances and haplotypes . . . ”, throughout the application. Instead, the italicized text in the foregoing sentence can be represented by the word “variance”, “variant” or “polymorphism”. Similarly, the term genotype, as used herein, means a procedure for determining the status of one or more variances in a gene, including a set of variances comprising a haplotype. Thus phrases such as “. . . genotype a patient . . . ” refer to determining the status of one or more variances, including a set of variances for which phase is known (i.e. a haplotype).

In preferred embodiments of this invention, the frequency of the variance or variant form of the gene in a population is known. Measures of frequency known in the art include “allele frequency”, namely the fraction of genes in a population that have one specific variance or set of variances. The allele frequencies for any gene should sum to 1. Another measure of frequency known in the art is the “heterozygote frequency” namely, the fraction of individuals in a population who carry two alleles, or two forms of a particular variance or variant form of a gene, one inherited from each parent. Alternatively, the number of individuals who are homozygous for a particular form of a gene may be a useful measure. The relationship between allele frequency, heterozygote frequency, and homozygote frequency is described for many genes by the Hardy-Weinberg equation, which provides the relationship between allele frequency, heterozygote frequency and homozygote frequency in a freely breeding population at equilibrium. Most human variances are substantially in Hardy-Weinberg equilibrium. In a preferred aspect of this invention, the allele frequency, heterozygote frequency, and homozygote frequencies are determined experimentally. Preferably a variance has an allele frequency of at least 0.01, more preferably at least 0.05, still more preferably at least 0.10. However, the allele may have a frequency as low as 0.001 if the associated phenotype is, for example, a rare form of toxic reaction to a treatment or drug. Beneficial responses may also be rare.

In this regard, “population” refers to a defined group of individuals or a group of individuals with a particular disease or condition or individuals that may be treated with a specific drug identified by, but not limited to geographic, ethnic, race, gender, and/or cultural indices. In most cases a population will preferably encompass at least ten thousand, one hundred thousand, one million, ten million, or more individuals, with the larger numbers being more preferable. In a preferred aspect of this invention, the population refers to individuals with a specific disease or condition that may be treated with a specific drug. In an aspect of this invention, the allele frequency, heterozygote frequency, or homozygote frequency of a specific variance or variant form of a gene is known. In preferred embodiments of this invention, the frequency of one or more variances that may predict response to a treatment is determined in one or more populations using a diagnostic test.

It should be emphasized that it is currently not generally practical to study an entire population to establish the association between a specific disease or condition or response to a treatment and a specific variance or variant form of a gene. Such studies are preferably performed in controlled clinical trials using a limited number of patients that are considered to be representative of the population with the disease. Since drug development programs are generally targeted at the largest possible population, the study population will generally consist of men and women, as well as members of various racial and ethnic groups, depending on where the clinical trial is being performed. This is important to establish the efficacy of the treatment in all segments of the population.

In the context of this invention, the term “probe” refers to a molecule which detectably distinguishes between target molecules differing in structure. Detection can be accomplished in a variety of different ways depending on the type of probe used and the type of target molecule. Thus, for example, detection may be based on discrimination of activity levels of the target molecule, but preferably is based on detection of specific binding. Examples of such specific binding include antibody binding and nucleic acid probe hybridization. Thus, for example, probes can include enzyme substrates, antibodies and antibody fragments, and nucleic acid hybridization probes. Thus, in preferred embodiments, the detection of the presence or absence of the at least one variance involves contacting a nucleic acid sequence which includes a variance site with a probe, preferably a nucleic acid probe, where the probe preferentially hybridizes with a form of the nucleic acid sequence containing a complementary base at the variance site as compared to hybridization to a form of the nucleic acid sequence having a non-complementary base at the variance site, where the hybridization is carried out under selective hybridization conditions. Such a nucleic acid hybridization probe may span two or more variance sites. Unless otherwise specified, a nucleic acid probe can include one or more nucleic acid analogs, labels or other substituents or moieties so long as the base-pairing function is retained.

As is generally understood, administration of a particular treatment, e.g., administration of a therapeutic compound or combination of compounds, is chosen depending on the disease or condition which is to be treated. Thus, in certain preferred embodiments, the disease or condition is one for which administration of a treatment is expected to provide a therapeutic benefit.

As used herein, the terms “effective” and “effectiveness” includes both pharmacological effectiveness and physiological safety. Pharmacological effectiveness refers to the ability of the treatment to result in a desired biological effect in the patient. Physiological safety refers to the level of toxicity, or other adverse physiological effects at the cellular, organ and/or organism level (often referred to as side-effects) resulting from administration of the treatment. On the other hand, the term “ineffective” indicates that a treatment does not provide sufficient pharmacological effect to be therapeutically useful, even in the absence of deleterious effects, at least in the unstratified population. (Such a treatment may be ineffective in a subgroup that can be identified by the presence of one or more sequence variances or alleles.) “Less effective” means that the treatment results in a therapeutically significant lower level of pharmacological effectiveness and/or a therapeutically greater level of adverse physiological effects, e.g., greater liver toxicity.

Thus, in connection with the administration of a drug, a drug which is “effective against” a disease or condition indicates that administration in a clinically appropriate manner results in a beneficial effect for at least a statistically significant fraction of patients, such as a improvement of symptoms, a cure, a reduction in disease load, reduction in tumor mass or cell numbers, extension of life, improvement in quality of life, or other effect generally recognized as positive by medical doctors familiar with treating the particular type of disease or condition.

Effectiveness is measured in a particular population. In conventional drug development the population is generally every subject who meets the enrollment criteria (i.e. has the particular form of the disease or condition being treated). It is an aspect of the present invention that segmentation of a study population by genetic criteria can provide the basis for identifying a subpopulation in which a drug is effective against the disease or condition being treated.

The term “deleterious effects” refers to physical effects in a patient caused by administration of a treatment which are regarded as medically undesirable. Thus, for example, deleterious effects can include a wide spectrum of toxic effects injurious to health such as death of normally functioning cells when only death of diseased cells is desired, nausea, fever, inability to retain food, dehydration, damage to critical organs such as arrythmias, renal tubular necrosis, fatty liver, or pulmonary fibrosis leading to coronary, renal, hepatic, or pulmonary insufficiency among many others. In this regard, the term “adverse reactions” refers to those manifestations of clinical symptomology of pathological disorder or dysfunction is induced by administration or a drug, agent, or candidate therapeutic intervention. In this regard, the term “contraindicated” means that a treatment results in deleterious effects such that a prudent medical doctor treating such a patient would regard the treatment as unsuitable for administration. Major factors in such a determination can include, for example, availability and relative advantages of alternative treatments, consequences of non-treatment, and permanency of deleterious effects of the treatment.

It is recognized that many treatment methods, e.g., administration of certain compounds or combinations of compounds, may produce side-effects or other deleterious effects in patients. Such effects can limit or even preclude use of the treatment method in particular patients, or may even result in irreversible injury, disorder, dysfunction, or death of the patient. Thus, in certain embodiments, the variance information is used to select both a first method of treatment and a second method of treatment. Usually the first treatment is a primary treatment which provides a physiological effect directed against the disease or condition or its symptoms. The second method is directed to reducing or eliminating one or more deleterious effects of the first treatment, e.g., to reduce a general toxicity or to reduce a side effect of the primary treatment. Thus, for example, the second method can be used to allow use of a greater dose or duration of the first treatment, or to allow use of the first treatment in patients for whom the first treatment would not be tolerated or would be contra-indicated in the absence of a second method to reduce deleterious effects or to potentiate the effectiveness of the first treatment.

In a related aspect, the invention provides a method for selecting a method of treatment for a patient suffering from a disease or condition by comparing at least one variance in at least one gene in the patient, with a list of variances in the gene from Tables 1, 3 and 4, or other gene related to pharmacokinetic parameters, or organ and tissue damage, or inordinate immune response, which are indicative of the effectiveness or safety of at least one method of treatment. Preferably the comparison involves a plurality of variances or a haplotype indicative of the effectiveness of at least one method of treatment. Also, preferably the list of variances includes a plurality of variances.

Similar to the above aspect, in preferred embodiments the at least one method of treatment involves the administration of a compound effective in at least some patients with a disease or condition; the presence or absence of the at least one variance is indicative that the treatment will be effective in the patient; and/or the presence or absence of the at least one variance is indicative that the treatment will be ineffective or contra-indicated in the patient; and/or the treatment is a first treatment and the presence or absence of the at least one variance is indicative that a second treatment will be beneficial to reduce a deleterious effect or potentiate the effectiveness of the first treatment; and/or the at least one treatment is a plurality of methods of treatment. For a plurality of treatments, preferably the selecting involves determining whether any of the methods of treatment will be more effective than at least one other of the plurality of methods of treatment. Yet other embodiments are provided as described for the preceding aspect in connection with methods of treatment using administration of a compound; treatment of various diseases, and variances in particular genes.

In the context of variance information in the methods of this invention, the term “list” refers to one or more variances which have been identified for a gene of potential importance in accounting for inter-individual variation in treatment response. Preferably there is a plurality of variances for the gene, preferably a plurality of variances for the particular gene. Preferably, the list is recorded in written or electronic form. For example, identified variances of identified genes are recorded for some of the genes in Tables 3 and 4, additional variances for genes in Table 1 are provided in Table 1 of Stanton & Adams, application Ser. No. 09/300,747, supra, and additional gene variance identification tables are provided in a form which allows comparison with other variance information. The possible additional variances in the identified genes are provided in Table 3 in Stanton & Adams, application Ser. No. 09/300,747, supra.

In addition to the basic method of treatment, often the mode of administration of a given compound as a treatment for a disease or condition in a patient is significant in determining the course and/or outcome of the treatment for the patient. Thus, the invention also provides a method for selecting a method of administration of a compound to a patient suffering from a disease or condition, by determining the presence or absence of at least one variance in cells of the patient in at least one identified gene from Tables 1, 3, and 4, where such presence or absence is indicative of an appropriate method of administration of the compound. Preferably, the selection of a method of treatment (a treatment regimen) involves selecting a dosage level or frequency of administration or route of administration of the compound or combinations of those parameters. In preferred embodiments, two or more compounds are to be administered, and the selecting involves selecting a method of administration for one, two, or more than two of the compounds, jointly, concurrently, or separately. As understood by those skilled in the art, such plurality of compounds may be used in combination therapy, and thus may be formulated in a single drug, or may be separate drugs administered concurrently, serially, or separately. Other embodiments are as indicated above for selection of second treatment methods, methods of identifying variances, and methods of treatment as described for aspects above.

In another aspect, the invention provides a method for selecting a patient for administration of a method of treatment for a disease or condition, or of selecting a patient for a method of administration of a treatment, by comparing the presence or absence of at least one variance in a gene as identified above in cells of a patient, with a list of variances in the gene, where the presence or absence of the at least one variance is indicative that the treatment or method of administration will be effective in the patient. If the at least one variance is present in the patient's cells, then the patient is selected for administration of the treatment.

In preferred embodiments, the disease or the method of treatment is as described in aspects above, specifically including, for example, those described for selecting a method of treatment.

In another aspect, the invention provides a method for identifying a subset of patients with enhanced or diminished response or tolerance to a treatment method or a method of administration of a treatment where the treatment is for a disease or condition in the patient. The method involves correlating one or more variances in one or more genes as identified in aspects above in a plurality of patients with response to a treatment or a method of administration of a treatment. The correlation may be performed by determining the one or more variances in the one or more genes in the plurality of patients and correlating the presence or absence of each of the variances (alone or in various combinations) with the patient's response to treatment. The variances may be previously known to exist or may also be determined in the present method or combinations of prior information and newly determined information may be used. The enhanced or diminished response should be statistically significant, preferably such that p=0.10 or less, more preferably 0.05 or less, and most preferably 0.02 or less. A positive correlation between the presence of one or more variances and an enhanced response to treatment is indicative that the treatment is particularly effective in the group of patients having those variances. A positive correlation of the presence of the one or more variances with a diminished response to the treatment is indicative that the treatment will be less effective in the group of patients having those variances. Such information is useful, for example, for selecting or de-selecting patients for a particular treatment or method of administration of a treatment, or for demonstrating that a group of patients exists for which the treatment or method of treatment would be particularly beneficial or contra-indicated. Such demonstration can be beneficial, for example, for obtaining government regulatory approval for a new drug or a new use of a drug

In preferred embodiments, the variances are in at least one of the identified genes listed in Tables 1, 3, and 4, or are particular variances described herein. Also, preferred embodiments include drugs, treatments, variance identification or determination, determination of effectiveness, and/or diseases as described for aspects above or otherwise described herein.

In preferred embodiments, the correlation of patient responses to therapy according to patient genotype is carried out in a clinical trial, e.g., as described herein according to any of the variations described. Detailed description of methods for associating variances with clinical outcomes using clinical trials are provided below. Further, in preferred embodiments the correlation of pharmacological effect (positive or negative) to treatment response according to genotype or haplotype in such a clinical trial is part of a regulatory submission to a government agency leading to approval of the drug. Most preferably the compound or compounds would not be approvable in the absence of the genetic information allowing identification of an optimal responder population.

As indicated above, in aspects of this invention involving selection of a patient for a treatment, selection of a method or mode of administration of a treatment, and selection of a patient for a treatment or a method of treatment, the selection may be positive selection or negative selection. Thus, the methods can include eliminating a treatment for a patient, eliminating a method or mode of administration of a treatment to a patient, or elimination of a patient for a treatment or method of treatment.

Also, in methods involving identification and/or comparison of variances present in a gene of a patient, the methods can involve such identification or comparison for a plurality of genes. Preferably, the genes are functionally related to the same disease or condition, or to the aspect of disease pathophysiology that is being subjected to pharmacological manipulation by the treatment (e.g., a drug), or to the activation or inactivation or elimination of the drug, and more preferably the genes are involved in the same biochemical process or pathway.

In another aspect, the invention provides a method for identifying the forms of a gene in an individual, where the gene is one specified as for aspects above, by determining the presence or absence of at least one variance in the gene. In preferred embodiments, the at least one variance includes at least one variance selected from the group of variances identified in variance tables herein. Preferably, the presence or absence of the at least one variance is indicative of the effectiveness of a therapeutic treatment in a patient suffering from a disease or condition and having cells containing the at least one variance.

The presence or absence of the variances can be determined in any of a variety of ways as recognized by those skilled in the art. For example, the nucleotide sequence of at least one nucleic acid sequence which includes at least one variance site (or a complementary sequence) can be determined, such as by chain termination methods, hybridization methods or by mass spectrometric methods. Likewise, in preferred embodiments, the determining involves contacting a nucleic acid sequence or a gene product of one of one of the genes with a probe which specifically identifies the presence or absence of a form of the gene. For example, a probe, e.g., a nucleic acid probe, can be used which specifically binds, e.g., hybridizes, to a nucleic acid sequence corresponding to a portion of the gene and which includes at least one variance site under selective binding conditions. As described for other aspects, determining the presence or absence of at least two variances and their relationship on the two gene copies present in a patient can constitute determining a haplotype or haplotypes.

Other preferred embodiments involve variances related to types of treatment, drug responses, diseases, nucleic acid sequences, and other items related to variances and variance determination as described for aspects above.

In yet another aspect, the invention provides a pharmaceutical composition which includes a compound which has a differential effect in patients having at least one copy, or alternatively, two copies of a form of a gene as identified for aspects above and a pharmaceutically acceptable carrier, excipient, or diluent. The composition is adapted to be preferentially effective to treat a patient with cells containing the one, two, or more copies of the form of the gene.

In preferred embodiments of aspects involving pharmaceutical compositions, active compounds, or drugs, the material is subject to a regulatory limitation, restriction, or recommendation on approved uses or indications, e.g., by the U.S. Food and Drug Administration (FDA), limiting or recommending limiting approved use of the composition to patients having at least one copy of the particular form of the gene which contains at least one variance. Alternatively, the composition is subject to a regulatory limitation, restriction, or recommendation on approved uses indicating or recommending that the composition is not approved for use or should not be used in patients having at least one copy of a form of the gene including at least one variance. Also in preferred embodiments, the composition is packaged, and the packaging includes a label or insert indicating or suggesting beneficial therapeutic approved use of the composition in patients having one or two copies of a form of the gene including at least one variance. Alternatively, the label or insert limits or recommends limiting approved use of the composition to patients having zero or one or two copies of a form of the gene including at least one variance. The latter embodiment would be likely where the presence of the at least one variance in one or two copies in cells of a patient means that the composition would be ineffective or deleterious to the patient. Also in preferred embodiments, the composition is indicated for use in treatment of a disease or condition that is one of those identified for aspects above. Also in preferred embodiments, the at least one variance includes at least one variance from those identified herein.

The term “packaged” means that the drug, compound, or composition is prepared in a manner suitable for distribution or shipping with a box, vial, pouch, bubble pack, or other protective container, which may also be used in combination. The packaging may have printing on it and/or printed material may be included in the packaging.

In preferred embodiments, the drug is selected from the drug classes or specific exemplary drugs identified in an example, in a table herein, and is subject to a regulatory limitation or suggestion or warning as described above that limits or suggests limiting approved use to patients having specific variances or variant forms of a gene identified in Examples or in the gene list provided below in order to achieve maximal benefit and avoid toxicity or other deleterious effect.

A pharmaceutical composition can be adapted to be preferentially effective in a variety of ways. In some cases, an active compound is selected which was not previously known to be differentially active, or which was not previously recognized as a therapeutic compound. Alternatively the compound was previously known as a therapeutic compound, but the composition is formulated in a manner appropriate for administration for treatment of a disease or condition for which a gene of this invention is involved in treatment response, and the active compound had not been formulated appropriately for such use before. For example, a compound may previously have been formulated for topical treatment of a skin condition, but is found to be effective in IV or other internal treatment of a disease identified for this invention. For compounds that are differentially effective on the gene, such alternative formulations are adapted to be preferentially effective. In some cases, the concentration of an active compound which has differential activity can be adjusted such that the composition is appropriate for administration to a patient with the specified variances. For example, the presence of a specified variance may allow or require the administration of a much larger dose, which would not be practical with a previously utilized composition. Conversely, a patient may require a much lower dose, such that administration of such a dose with a prior composition would be impractical or inaccurate. Thus, the composition may be prepared in a higher or lower unit dose form, or prepared in a higher or lower concentration of the active compound or compounds. In yet other cases, the composition can include additional compounds useful to enable administration of a particular active compound in a patient with the specified variances, which was not in previous compositions, e.g., because the majority of patients did not require or benefit from the added component.

The term “differential” or “differentially” generally refers to a statistically significant different level in the specified property or effect. Preferably, the difference is also functionally significant. Thus, “differential binding or hybridization” is sufficient difference in binding or hybridization to allow discrimination using an appropriate detection technique. Likewise, “differential effect” or “differentially active” in connection with a therapeutic treatment or drug refers to a difference in the level of the effect or activity which is distinguishable using relevant parameters and techniques for measuring the effect or activity being considered. Preferably the difference in effect or activity is also sufficient to be clinically significant, such that a corresponding difference in the course of treatment or treatment outcome would be expected, at least on a statistical basis.

Also usefully provided in the present invention are probes which specifically recognize a nucleic acid sequence corresponding to a variance or variances in a gene as identified in aspects above or a product expressed from the gene, and are able to distinguish a variant form of the sequence or gene or gene product from one or more other variant forms of that sequence, gene, or gene product under selective conditions. Those skilled in the art recognize and understand the identification or determination of selective conditions for particular probes or types of probes. An exemplary type of probe is a nucleic acid hybridization probe, which will selectively bind under selective binding conditions to a nucleic acid sequence or a gene product corresponding to one of the genes identified for aspects above. Another type of probe is a peptide or protein, e.g., an antibody or antibody fragment which specifically or preferentially binds to a polypeptide expressed from a particular form of a gene as characterized by the presence or absence of at least one variance. Thus, in another aspect, the invention concerns such probes. In the context of this invention, a “probe” is a molecule, commonly a nucleic acid, though also potentially a protein, carbohydrate, polymer, or small molecule, that is capable of binding to one variance or variant form of the gene to a greater extent than to a form of the gene having a different base at one or more variance sites, such that the presence of the variance or variant form of the gene can be determined. Preferably the probe distinguishes at least one variance identified in Examples, tables or lists below or in Tables 1 or 3 of Stanton & Adams, application Ser. No. 09/300,747, supra.

In preferred embodiments, the probe is a nucleic acid probe 6, 7, 8, 9, 10, 11, 12, 13, 14 or preferably at least 17 nucleotides in length, more preferably at least 20 or 22 or 25, preferably 500 or fewer nucleotides in length, more preferably 200 or 100 or fewer, still more preferably 50 or fewer, and most preferably 30 or fewer. In preferred embodiments, the probe has a length in a range from any one of the above lengths to any other of the above lengths (including endpoints). The probe specifically hybridizes under selective hybridization conditions to a nucleic acid sequence corresponding to a portion of one of the genes identified in connection with above aspects. The nucleic acid sequence includes at least one and preferably two or more variance sites. Also in preferred embodiments, the probe has a detectable label, preferably a fluorescent label. A variety of other detectable labels are known to those skilled in the art. Such a nucleic acid probe can also include one or more nucleic acid analogs.

In preferred embodiments, the probe is an antibody or antibody fragment which specifically binds to a gene product expressed from a form of one of the above genes, where the form of the gene has at least one specific variance with a particular base at the variance site, and preferably a plurality of such variances.

In connection with nucleic acid probe hybridization, the term “specifically hybridizes” indicates that the probe hybridizes to a sufficiently greater degree to the target sequence than to a sequence having a mismatched base at least one variance site to allow distinguishing such hybridization. The term “specifically hybridizes” thus means that the probe hybridizes to the target sequence, and not to non-target sequences, at a level which allows ready identification of probe/target sequence hybridization under selective hybridization conditions. Thus, “selective hybridization conditions” refer to conditions which allow such differential binding. Similarly, the terms “specifically binds” and “selective binding conditions” refer to such differential binding of any type of probe, e.g., antibody probes, and to the conditions which allow such differential binding. Typically hybridization reactions to determine the status of variant sites in patient samples are carried out with two different probes, one specific for each of the (usually two) possible variant nucleotides. The complementary information derived from the two separate hybridization reactions is useful in corroborating the results.

Likewise, the invention provides an isolated, purified or enriched nucleic acid sequence of 15 to 500 nucleotides in length, preferably 15 to 100 nucleotides in length, more preferably 15 to 50 nucleotides in length, and most preferably 15 to 30 nucleotides in length, which has a sequence which corresponds to a portion of one of the genes identified for aspects above. Preferably the lower limit for the preceding ranges is 17, 20, 22, or 25 nucleotides in length. In other embodiments, the nucleic acid sequence is 30 to 300 nucleotides in length, or 45 to 200 nucleotides in length, or 45 to 100 nucleotides in length. The nucleic acid sequence includes at least one variance site. Such sequences can, for example, be amplification products of a sequence which spans or includes a variance site in a gene identified herein. Likewise, such a sequence can be a primer, or amplification oligonucleotide which is able to bind to or extend through a variance site in such a gene. Yet another example is a nucleic acid hybridization probe comprised of such a sequence. In such probes, primers, and amplification products, the nucleotide sequence can contain a sequence or site corresponding to a variance site or sites, for example, a variance site identified herein. Preferably the presence or absence of a particular variant form in the heterozygous or homozygous state is indicative of the effectiveness of a method of treatment in a patient.

Likewise, the invention provides a set of primers or amplification oligonucleutides (e.g., 2, 3, 4, 6, 8, 10 or even more) adapted for binding to or extending through at least one gene identified herein. In preferred embodiments the set includes primers or amplification oligonucleotides adapted to bind to or extend through a plurality of sequence variances in a gene(s) identified herein. The plurality of variances preferably provides a haplotype. Those skilled in the art are familiar with the use of amplification oligonucleotides (e.g., PCR primers) and the appropriate location, testing and use of such oligonucleotides. In certain embodiments, the oligonucleotides are designed and selected to provide variance-specific amplification.

In reference to nucleic acid sequences which “correspond” to a gene, the term “correspond” refers to a nucleotide sequence relationship, such that the nucleotide sequence has a nucleotide sequence which is the same as the reference gene or an indicated portion thereof, or has a nucleotide sequence which is exactly complementary in normal Watson-Crick base pairing, or is an RNA equivalent of such a sequence, e.g., an mRNA, or is a cDNA derived from an mRNA of the gene.

In another aspect, the invention provides a kit containing at least one probe or at least one primer (or other amplification oligonucleotide) or both (e.g., as described above) corresponding to a gene or genes listed in Tables 1, 3, and 4 or other gene related to a drug-induced disease or condition, or other gene involved in absorption, distribution, metabolism, excretion, or in toxicity-related modification of a drug. The kit is preferably adapted and configured to be suitable for identification of the presence or absence of a particular variance or variances, which can include or consist of a nucleic acid sequence corresponding to a portion of a gene. A plurality of variances may comprise a haplotype of haplotypes. The kit may also contain a plurality of either or both of such probes and/or primers, e.g., 2, 3, 4, 5, 6, or more of such probes and/or primers. Preferably the plurality of probes and/or primers are adapted to provide detection of a plurality of different sequence variances in a gene or plurality of genes, e.g., in 2, 3, 4, 5, or more genes or to amplify and/or sequence a nucleic acid sequence including at least one variance site in a gene or genes. Preferably one or more of the variance or variances to be detected are correlated with variability in a treatment response or tolerance, and are preferably indicative of an effective response to a treatment. In preferred embodiments, the kit contains components (e.g., probes and/or primers) adapted or useful for detection of a plurality of variances (which may be in one or more genes) indicative of the effectiveness of at least one treatment, preferably of a plurality of different treatments for a particular disease or condition. It may also be desirable to provide a kit containing components adapted or useful to allow detection of a plurality of variances indicative of the effectiveness of a treatment or treatment against a plurality of diseases. The kit may also optionally contain other components, preferably other components adapted for identifying the presence of a particular variance or variances. Such additional components can, for example, independently include a buffer or buffers, e.g., amplification buffers and hybridization buffers, which may be in liquid or dry form, a DNA polymerase, e.g., a polymerase suitable for carrying out PCR (e.g., a thermostable DNA polymerase), and deoxy nucleotide triphosphates (dNTPs). Preferably a probe includes a detectable label, e.g., a fluorescent label, enzyme label, light scattering label, or other label. Preferably the kit includes a nucleic acid or polypeptide array on a solid phase substrate. The array may, for example, include a plurality of different antibodies, and/or a plurality of different nucleic acid sequences. Sites in the array can allow capture and/or detection of nucleic acid sequences or gene products corresponding to different variances in one or more different genes. Preferably the array is arranged to provide variance detection for a plurality of variances in one or more genes which correlate with the effectiveness of one or more treatments of one or more diseases, which is preferably a variance as described herein.

The kit may also optionally contain instructions for use, which can include a listing of the variances correlating with a particular treatment or treatments for a disease or diseases and/or a statement or listing of the diseases for which a particular variance or variances correlates with a treatment efficacy and/or safety.

Preferably the kit components are selected to allow detection of a variance described herein, and/or detection of a variance indicative of a treatment, e.g., administration of a drug, pointed out herein.

Additional configurations for kits of this invention will be apparent to those skilled in the art.

The invention also includes the use of such a kit to determine the genotype(s) of one or more individuals with respect to one or more variance sites in one or more genes identified herein. Such use can include providing a result or report indicating the presence and/or absence of one or more variant forms or a gene or genes which are indicative of the effectiveness of a treatment or treatments.

In another aspect, the invention provides a method for determining a genotype of an individual in relation to one or more variances in one or more of the genes identified in above aspects by using mass spectrometric determination of a nucleic acid sequence which is a portion of a gene identified for other aspects of this invention or a complementary sequence. Such mass spectrometric methods are known to those skilled in the art. In preferred embodiments, the method involves determining the presence or absence of a variance in a gene; determining the nucleotide sequence of the nucleic acid sequence; the nucleotide sequence is 100 nucleotides or less in length, preferably 50 or less, more preferably 30 or less, and still more preferably 20 nucleotides or less. In general, such a nucleotide sequence includes at least one variance site, preferably a variance site which is informative with respect to the expected response of a patient to a treatment as described for above aspects.

As indicated above, many therapeutic compounds or combinations of compounds or pharmaceutical compositions show variable efficacy and/or safety in various patients in whom the compound or compounds is administered. Thus, it is beneficial to identify variances in relevant genes, e.g., genes related to the action or toxicity of the compound or compounds. Thus, in a further aspect, the invention provides a method for determining whether a compound has a differential effect due to the presence or absence of at least one variance in a gene or a variant form of a gene, where the gene is a gene identified for aspects above.

The method involves identifying a first patient or set of patients suffering from a disease or condition whose response to a treatment differs from the response (to the same treatment) of a second patient or set of patients suffering from the same disease or condition, and then determining whether the occurrence or frequency of occurrence of at least one variance in at least one gene differs between the first patient or set of patients and the second patient or set of patients. A correlation between the presence or absence of the variance or variances and the response of the patient or patients to the treatment indicates that the variance provides information about variable patient response. In general, the method will involve identifying at least one variance in at least one gene. An alternative approach is to identify a first patient or set of patients suffering from a disease or condition and having a particular genotype, haplotype or combination of genotypes or haplotypes, and a second patient or set of patients suffering from the same disease or condition that have a genotype or haplotype or sets of genotypes or haplotypes that differ in a specific way from those of the first set of patients. Subsequently the extent and magnitude of clinical response can be compared between the first patient or set of patients and the second patient or set of patients. A correlation between the presence or absence of a variance or variances or haplotypes and the response of the patient or patients to the treatment indicates that the variance provides information about variable patient response and is useful for the present invention.

The method can utilize a variety of different informative comparisons to identify correlations. For example a plurality of pairwise comparisons of treatment response and the presence or absence of at least one variance can be performed for a plurality of patients. Likewise, the method can involve comparing the response of at least one patient homozygous for at least one variance with at least one patient homozygous for the alternative form of that variance or variances. The method can also involve comparing the response of at least one patient heterozygous for at least one variance with the response of at least one patient homozygous for the at least one variance. Preferably the heterozygous patient response is compared to both alternative homozygous forms, or the response of heterozygous patients is grouped with the response of one class of homozygous patients and said group is compared to the response of the alternative homozygous group.

Such methods can utilize either retrospective or prospective information concerning treatment response variability. Thus, in a preferred embodiment, it is previously known that patient response to the method of treatment is variable.

Also in preferred embodiments, the disease or condition is as for other aspects of this invention; for example, the treatment involves administration of a compound or pharmaceutical composition.

In preferred embodiments, the method involves a clinical trial, e.g., as described herein. Such a trial can be arranged, for example, in any of the ways described herein, e.g., in the Detailed Description.

The present invention also provides methods of treatment of a disease or condition, preferably a disease or condition related to pharmacokinetic parameters, e.g. absorption, distribution, metabolism, or excretion, that affect a drug or candidate therapeutic intervention regarding efficacy and or safety, i.e. drug-induced disease, disorder or dysfunction or other toxicity effects or clinical symptomatology. Such methods combine identification of the presence or absence of particular variances, preferably in a gene or genes from Tables 1, 3, and 4, with the administration of a compound; identification of the presence of particular variances with selection of a method of treatment and administration of the treatment; and identification of the presence or absence of particular variances with elimination of a method of treatment based on the variance information indicating that the treatment is likely to be ineffective or contra-indicated, and thus selecting and administering an alternative treatment effective against the disease or condition. Thus, preferred embodiments of these methods incorporate preferred embodiments of such methods as described for such sub-aspects.

As used herein, a “gene” is a sequence of DNA present in a cell that directs the expression of a “biologically active” molecule or “gene product”, most commonly by transcription to produce RNA and translation to produce protein. The “gene product’ is most commonly a RNA molecule or protein or a RNA or protein that is subsequently modified by reacting with, or combining with, other constituents of the cell. Such modifications may include, without limitation, modification of proteins to form glycoproteins, lipoproteins, and phosphoproteins, or other modifications known in the art. RNA may be modified without limitation by polyadenylation, splicing, capping or export from the nucleus or by covalent or noncovalent interactions with proteins. The term “gene product” refers to any product directly resulting from transcription of a gene. In particular this includes partial, precursor, and mature transcription products (i.e., pre-mRNA and mRNA), and translation products with or without further processing including, without limitation, lipidation, phosphorylation, glycosylation, or combinations of such processing

The term “gene involved in the origin or pathogenesis of a disease or condition” refers to a gene that harbors mutations or polymorphisms that contribute to the cause of disease, or variances that affect the progression of the disease or expression of specific characteristics of the disease. The term also applies to genes involved in the synthesis, accumulation, or elimination of products that are involved in the origin or pathogenesis of a disease or condition including, without limitation, proteins, lipids, carbohydrates, hormones, or small molecules.

The term “gene involved in the action of a drug” refers to any gene whose gene product affects the efficacy or safety of the drug or affects the disease process being treated by the drug, and includes, without limitation, genes that encode gene products that are targets for drug action, gene products that are involved in the metabolism, activation or degradation of the drug, gene products that are involved in the bioavailability or elimination of the drug to the target, gene products that affect biological pathways that, in turn, affect the action of the drug such as the synthesis or degradation of competitive substrates or allosteric effectors or rate-limiting reaction, or, alternatively, gene products that affect the pathophysiology of the disease process via pathways related or unrelated to those altered by the presence of the drug compound. (Particular variances in the latter category of genes may be associated with patient groups in whom disease etiology is more or less susceptible to amelioration by the drug. For example, there are several pathophysiological mechanisms in hypertension, and depending on the dominant mechanism in a given patient, that patient may be more or less likely than the average hypertensive patient to respond to a drug that primarily targets one pathophysiological mechanism. The relative importance of different pathophysiological mechanisms in individual patients is likely to be affected by variances in genes associated with the disease pathophysiology.) The “action” of a drug refers to its effect on biological products within the body. The action of a drug also refers to its effects on the signs or symptoms of a disease or condition, or effects of the drug that are unrelated to the disease or condition leading to unanticipated effects on other processes. Such unanticipated processes often lead to adverse events or toxic effects. The terms “adverse event” or “toxic” event” are known in the art and include, without limitation, those listed in the FDA reference system for adverse events.

In accordance with the aspects above and the Detailed Description below, there is also described for this invention an approach for developing drugs that are explicitly indicated for, and/or for which approved use is restricted to individuals in the population with specific variances or combinations of variances, as determined by diagnostic tests for variances or variant forms of certain genes involved in the disease or condition or involved in the action or metabolism or transport of the drug. Such drugs may provide more effective treatment for a disease or condition in a population identified or characterized with the use of a diagnostic test for a specific variance or variant form of the gene if the gene is involved in the action of the drug or in determining a characteristic of the disease or condition. Such drugs may be developed using the diagnostic tests for specific variances or variant forms of a gene to determine the inclusion of patients in a clinical trial.

Thus, the invention also provides a method for producing a pharmaceutical composition by identifying a compound which has differential activity or effectiveness against a disease or condition in patients having at least one variance in a gene, preferably in a gene from Tables 1, 3 and 4, compounding the pharmaceutical composition by combining the compound with a pharmaceutically acceptable carrier, excipient, or diluent such that the composition is preferentially effective in patients who have at least one copy of the variance or variances. In some cases, the patient has two copies of the variance or variances. In preferred embodiments, the disease or condition, gene or genes, variances, methods of administration, or method of determining the presence or absence of variances is as described for other aspects of this invention.

Similarly, the invention provides a method for producing a pharmaceutical agent by identifying a compound which has differential activity against a disease or condition in patients having at least one copy of a form of a gene, preferably a gene listed in Table 1, having at least one variance and synthesizing the compound in an amount sufficient to provide a pharmaceutical effect in a patient suffering from the disease or condition. The compound can be identified by conventional screening methods and its activity confirmed. For example, compound libraries can be screened to identify compounds which differentially bind to products of variant forms of a particular gene product, or which differentially affect expression of variant forms of the particular gene, or which differentially affect the activity of a product expressed from such gene. Alternatively, the design of a compound can exploit knowledge of the variances provided herein to avoid significant allele specific effects, in order to reduce the likelihood of significant pharmacogenetic effects during the clinical development process. Preferred embodiments are as for the preceding aspect.

In another aspect, the invention provides a method of treating a disease or condition in a patient by selecting a patient whose cells have an allele of an identified gene, preferably a gene selected from the genes listed in Table 1, and determining whether that alteration provides a differential effect (with respect to reducing or alleviating a disease or condition, or with respect to variation in toxicity or tolerance to a treatment) in patients with at least one copy of at least one allele of the gene as compared to patients with at least one copy of one alternative allele. The presence of such a differential effect indicates that altering the level or activity of the gene provides at least part of an effective treatment for the disease or condition.

Preferably the allele contains a variance as shown in Tables 3 and 4 or other variance table herein, or in Table 1 or 3 of Stanton & Adams, application Ser. No. 09/300,747, supra. Also preferably, the altering involves administering to the patient a compound preferentially active on at least one but less than all alleles of the gene.

Preferred embodiments include those as described above for other aspects of treating a disease or condition.

As recognized by those skilled in the art, all the methods of treating described herein include administration of the treatment to a patient.

In a further aspect, the invention provides a method for determining a method of treatment effective to treat a disease or condition by altering the level of activity of a product of an allele of a gene selected from the genes listed in Tables 1, 3 or 4, and determining whether that alteration provides a differential effect related to reducing or alleviating a disease or condition as compared to at least one alternative allele or an alteration in toxicity or tolerance of the treatment by a patient or patients. The presence of such a differential effect indicates that altering that level of activity provides at least part of an effective treatment for the disease or condition.

Preferably the method for determining a method of treatment is carried out in a clinical trial, e.g., as described above and/or in the Detailed Description below.

In still another aspect, the invention provides a method for evaluating differential efficacy of or tolerance to a treatment in a subset of patients who have a particular variance or variances in at least one gene, preferably a gene in Tables 1, 3, or 4, by utilizing a clinical trial. In preferred embodiments, the clinical trial is a Phase I, II, III, or IV trial. Preferred embodiments include the stratifications and/or statistical analyses as described below in the Detailed Description.

In yet another aspect, the invention provides experimental methods for finding additional variances in a gene provided in Tables 3 and 4. A number of experimental methods can also beneficially be used to identify variances. Thus, the invention provides methods for producing cDNA (Example 12) and detecting additional variances in the genes provided in Tables 1 and 2 using the single strand conformation polymorphism (SSCP) method (Example 13), the T4 Endonuclease VII method (Example 14) or DNA sequencing (Example 15) or other methods pointed out below. The application of these methods to the identified genes will provide identification of additional variances that can affect inter-individual variation in drug or other treatment response. One skilled in the art will recognize that many methods for experimental variance detection have been described (in addition to the exemplary methods of examples 13, 14, and 15) which can be utilized. These additional methods include chemical cleavage of mismatches (see, e.g., Ellis T P, et al., Chemical cleavage of mismatch: a new look at an established method. Human Mutation 11(5):345-53, 1998), denaturing gradient gel electrophoresis (see, e.g., Van Orsouw N J, et al., Design and application of 2-D DGGE-based gene mutational scanning tests. Genet Anal. 14(5-6):205-13, 1999) and heteroduplex analysis (see, e.g., Ganguly A, et al., Conformation-sensitive gel electrophoresis for rapid detection of single-base differences in double-stranded PCR products and DNA fragments: evidence for solvent-induced bends in DNA heteroduplexes. Proc Natl Acad Sci U S A. 90 (21):10325-9, 1993). Table 3 of Stanton & Adams, application Ser. No. 09/300,747, supra, provides a description of the additional possible variances that could be detected by one skilled in the art by testing an identified gene in Tables 1 and 2 using the variance detection methods described or other methods which are known or are developed.

The present invention provides a method for treating a patient at risk for drug responsiveness, i.e., efficacy differences associated with pharmacokinetic parameters, and safety concerns, i.e. drug-induced disease, disorder, or dysfunction or diagnosed with organ failure or a disease associated with drug-induced organ failure. The methods include identifying such a patient and determining the patient's genotype or haplotype for an identified gene or genes. The patient identification can, for example, be based on clinical evaluation using conventional clinical metrics and/or evaluation of a genetic variance or variances in one or more genes, preferably a gene or genes from Tables 1, 3 and 4. The invention provides a method for using the patient's genotype status to determine a treatment protocol which includes a prediction of the efficacy and safety of a therapy for concurrent treatment in light of drug-induced disease or an drug-induced or drug associated pathological condition. In a related aspect, the invention features a treatment protocol that provides a prediction of patient outcome. Such predictions are based on a demonstrated correlation between a particular type of treatment and outcome, efficacy, safety, likelihood of development of drug-induced disease, disorder, or dysfunction, or other such parameter relevant to clinical treatment decisions as evaluated by a normal prudent physician.

In an another related aspect, the invention provides a method for identifying a patient for participation in a clinical trial of a therapy for the treatment of drug-induced disease, disorder, or dysfunction, or an associated drug-induced toxicity. The method involves determining the genotype or haplotype of a patient with (or at risk for) a drug-induced disease, disorder, or dysfunction. Preferably the genotype is for a variance in a gene from Table 1. Patients with eligible genotypes are then assigned to a treatment or placebo group, preferably by a blinded randomization procedure. In preferred embodiments, the selected patients have no copies, one copy or two copies of a specific allele of a gene or genes identified in Table 1. Alternatively, patients selected for the clinical trial may have zero, one or two copies of an allele belonging to a set of alleles, where the set of alleles comprise a group of related alleles. One procedure for rigorously defining a set of alleles is by applying phylogenetic methods to the analysis of haplotypes. (See, for example: Templeton A. R., Crandall K. A. and C. F. Sing A cladistic analysis of phenotypic associations with haplotypes inferred from restriction endonuclease mapping and DNA sequence data. III. Cladogram estimation. Genetics 1992 Oct;132(2):619-33.) Regardless of the specific tools used to group alleles, the trial would then test the hypothesis that a statistically significant difference in response to a treatment can be demonstrated between two groups of patients each defined by the presence of zero, one or two alleles (or allele groups) at a gene or genes. Said response may be a desired or an undesired response. In a preferred embodiment, the treatment protocol involves a comparison of placebo vs. treatment response rates in two or more genotype-defined groups. For example a group with no copies of an allele may be compared to a group with two copies, or a group with no copies may be compared to a group consisting of those with one or two copies. In this manner different genetic models (dominant, co-dominant, recessive) for the transmission of a treatment response trait can be tested. Alternatively, statistical methods that do not posit a specific genetic model, such as contingency tables, can be used to measure the effects of an allele on treatment response.

In another preferred embodiment, patients in a clinical trial can be grouped (at the end of the trial) according to treatment response, and statistical methods can be used to compare allele (or genotype or haplotype) frequencies in two groups. For example responders can be compared to nonresponders, or patients suffering adverse events can be compared to those not experiencing such effects. Alternatively response data can be treated as a continuous variable and the ability of genotype to predict response can be measured. In a preferred embodiments patients who exhibit extreme phenotypes are compared with all other patients or with a group of patients who exhibit a divergent extreme phenotype. For example if there is a continuous or semi-continuous measure of treatment response (for example the Alzheimer's Disease Assessment Scale, the Mini-Mental State Examination or the Hamilton Depression Rating Scale) then the 10% of patients with the most favorable responses could be compared to the 10% with the least favorable, or the patients one standard deviation above the mean score could be compared to the remainder, or to those one standard deviation below the mean score. One useful way to select the threshold for defining a response is to examine the distribution of responses in a placebo group. If the upper end of the range of placebo responses is used as a lower threshold for an ‘outlier response’ then the outlier response group should be almost free of placebo responders. This is a useful threshold because the inclusion of placebo responders in a ‘true’ response group decreases the ability of statistical methods to detect a genetic difference between responders and nonresponders.

In a related aspect, the invention provides a method for developing a disease management protocol that entails diagnosing a patient with a disease or a disease susceptibility, determining the genotype of the patient at a gene or genes correlated with treatment response and then selecting an optimal treatment based on the disease and the genotype (or genotypes or haplotypes). The disease management protocol may be useful in an education program for physicians, other caregivers or pharmacists; may constitute part of a drug label; or may be useful in a marketing campaign.

By “disease management protocol” or “treatment protocol” is meant a means for devising a therapeutic plan for a patient using laboratory, clinical and genetic data, including the patient's diagnosis and genotype. The protocol clarifies therapeutic options and provides information about probable prognoses with different treatments. The treatment protocol may provide an estimate of the likelihood that a patient will respond positively or negatively to a therapeutic intervention. The treatment protocol may also provide guidance regarding optimal drug dose and administration and likely timing of recovery or rehabilitation. A “disease management protocol” or “treatment protocol” may also be formulated for asymptomatic and healthy subjects in order to forecast future disease risks based on laboratory, clinical and genetic variables. In this setting the protocol specifies optimal preventive or prophylactic interventions, including use of compounds, changes in diet or behavior, or other measures. The treatment protocol may include the use of a computer program.

In preferred embodiments, the method further involves determining the patient's allele status and selecting those patients having at least one wild type allele, preferably having two wild type alleles for an identified gene, as candidates likely to develop drug-induced pathological conditions or drug-associated pathological disease or conditions. In a preferred embodiment, the treatment protocol involves a comparison of the allele status of a patient with a control population and a responder population. This comparison allows for a statistical calculation of a patient's likelihood of responding to a therapy, e.g., a calculation of the correlation between a particular allele status and treatment response. In the context of this aspect, the term “wild-type allele” refers to an allele of a gene which produces a product having a level of activity which is most common in the general population. Two different alleles may both be wild-type alleles for this purpose if both have essentially the same level of activity (e.g., specific activity and numbers of active molecules).

In preferred embodiments of above aspects involving prediction of drug efficacy, the prediction of drug efficacy involves candidate therapeutic interventions that are known or have been identified to be affected by pharmacokinetic parameters, i.e. absorption, distribution, metabolism, or excretion. These parameters may be associated with hepatic or extra-hepatic biological mechanisms. Preferably the candidate therapeutic intervention will be effective in patients with the genotype of a least one allele, and preferably two alleles from Tables 1, 3 and 4, but have a risk of drug ineffectiveness, i.e. nonresponsive to a drug or candidate therapeutic intervention.

In particular applications of the invention, all of the above aspects involving a gene variance evaluation or treatment selection or patient selection or method of treatment, the method includes a determination of the genotypic allele status of the patient, where a determination of the patient's allele status as being heterozygous or homozygous, is predictive of the patient having a poor response to a candidate therapeutic intervention and development of drug-induced disease, disorder, or dysfunction.

In preferred embodiments, the above methods are used for or include identification of a safety or toxicity concern involving a drug-induced disease, disorder, or dysfunction and/or the likelihood of occurrence and/or severity of said disease, disorder, or dysfunction.

In preferred embodiments, the invention is suitable for identifying a patient with non-drug-induced disease, disorder, or dysfunction but with dysfunction related to aberrant enzymatic metabolism or excretion of endogenous biologically relevant molecules or compounds. The method preferably involves determination of the allele status or variance presence or absence determination for at least one gene from Tables 1, 3, 4.

In another aspect, the invention provides a method for treating a patient at risk for a drug-induced disease, disorder or dysfunction by a) identifying a patient with such a risk, b) determining the genotypic allele status of the patient, and c) converting the data obtained in step b) into a treatment protocol that includes a comparison of the genotypic allele status determination with the allele frequency of a control population. This comparison allows for a statistical calculation of the patient's risk for having drug-induced disease, disorder, or dysfunction, e.g., based on correlation of the allele frequencies for a population with response or disease occurrence and/or severity. In preferred embodiments, the method provides a treatment protocol that predicts a patient being heterozygous or homozygous for an identified allele to exhibit signs and or symptoms of drug-induced disease, disorder, or dysfunction and a patient who is wild-type homozygous for the said allele, as responding favorably to these therapies.

In a related aspect, the invention provides a method for treating a patient at risk for or diagnosed with drug-induced disease or pathological condition or dysfunction using the methods of the above aspect and conducting a step c) which involves determining the gene allele load status of the patient. This method further involves converting the data obtained in steps b) and c) into a treatment protocol that includes a comparison of the allele status determinations of these steps with the allele frequency of a control population. This affords a statistical calculation of the patient's risk for having drug-induced disease, disorder or dysfunction. In a preferred embodiment, the method is useful for identifying drug-induced disease, disorder or dysfunction. In addition, in related embodiments, the methods provide a treatment protocol that predicts a patient to be at high risk for drug-induced disease, disorder or dysfunction responding by exhibiting signs and symptoms of drug-induced toxicity, disorders, dysfunction if the patient is determined as having a genotype or allelic difference in the identified gene or genes. Such patients are preferably given alternative therapies.

The invention also provides a method for improving the safety of candidate therapies for the identification of a drug-induced disease, disorder, or dysfunction. The method includes the step of comparing the relative safety of the candidate therapeutic intervention in patients having different alleles in one or more than one of the genes listed in Tables 1, 3, and 4. Preferably, administration of the drug is preferentially provided to those patients with an allele type associated with increased efficacy. In a preferred embodiment, the alleles of identified gene or genes used are wild-type and those associated with altered biological activity.

As used herein, by “therapy associated with drug-induced disease” is meant any therapy resulting in pathophysiologic dysfunction or signs and symptoms of failure or dysfunction, or those associated with the pathophysiological manifestations of a disorder. A suitable therapy can be a pharmacological agent, drug, or therapy that alters a pathways identified to affect the molecular structure or function of the parent candidate therapeutic intervention thereby affecting drug-induced disease or disorder progression of any of the described organ system dysfunctions.

By “drug-induced disease” or “drug-induced syndrome” is meant any physiologic condition that may be correlated with medical therapy by a drug, agent, or candidate therapeutic intervention.

By “drug-induced dysfunction” is meant a physiologic disorder or syndrome that may be correlated with medical therapy by a drug, agent, or candidate therapeutic intervention in which symptomology is similar to drug-induced disease. Specifically included are: a) hemostasis dysfunction; b) cutaneous disorders; c) cardiovascular dysfunction; d) renal dysfunction; e) pulmonary dysfunction; f) hepatic dysfunction; g) systemic reactions; and h) central nervous system dysfunction.

By “drug associated disorder” is meant a physiologic dysfunction that may be correlated with medical therapy by a drug, agent, or candidate therapeutic intervention. The drug associated disorder may include disease, disorder, or dysfunction.

By “pathway” or “gene pathway” is meant the group of biologically relevant genes involved in a pharmacodynamic or pharmacokinetic mechanism of drug, agent, or candidate therapeutic intervention. These mechanisms may further include any physiologic effect the drug or candidate therapeutic intervention renders.

As used herein, a “clinical trial” is the testing of a therapeutic intervention in a volunteer human population for the purpose of determining whether a therapeutic intervention is safe and/or efficacious in the human volunteer or patient population for a given disease, disorder, or condition. The analysis of safety and efficacy in genetically defined subgroups differing by at least one variance is of particular interest.

As used herein “clinical study” is that part of a clinical trial that involves determination of the effect a candidate therapeutic intervention on human subjects. It includes clinical evaluations of physiologic responses including pharmacokinetic (absorption, distribution, bioavailability, and excretion) as well as pharmacodynamic (physiologic response and efficacy) parameters. A pharmacogenetic clinical study is a clinical study that involves testing of one or more specific hypotheses regarding the effect of a genetic variance or variances (or set of variances, i.e. haplotype or haplotypes) in enrolled subjects or patients on response to a therapeutic intervention. These hypotheses are articulated before the study in the form of primary or secondary endpoints. For example the endpoint may be that in a particular genetic subgroup the rate of objectively defined responses exceeds some predefined threshold.

As used herein, “supplemental applications” are those in which a candidate therapeutic intervention is tested in a human clinical trial in order for the product to have an expanded label to include additional indications for therapeutic use. In these cases, the previous clinical studies of the therapeutic intervention, i.e. those involving the preclinical safety and Phase I human safety studies can be used to support the testing of the particular candidate therapeutic intervention in a patient population for a different disease, disorder, or condition than that previously approved in the U.S. In these cases, a limited Phase II study is performed in the proposed patient population. With adequate signs of efficacy, a Phase III study is designed. All other parameters of clinical development for this category of candidate therapeutic interventions proceeds as described above for interventions first tested in human candidates.

As used herein, “outcomes” or “therapeutic outcomes” are used to describe the results and value of healthcare intervention. Outcomes can be multi-dimensional, e.g., including one or more of the following: improvement of symptoms; regression of the disease, disorder, or condition; economic outcomes of healthcare decisions.

As used herein, “pharmacoeconomics” is the analysis of a therapeutic intervention in a population of patients diagnosed with a disease, disorder, or condition that includes at least one of the following studies: cost of illness study (COI); cost benefit analysis (CBA), cost minimization analysis (CMA), or cost utility analysis (CUA), or an analysis comparing the relative costs of a therapeutic intervention with one or a group of other therapeutic interventions. In each of these studies, the cost of the treatment of a disease, disorder, or condition is compared among treatment groups. As used herein, costs are those economic variables associated with a disease, disorder, or condition fall into two broad categories: direct and indirect. Direct costs are associated with the medical and non-medical resources used as therapeutic interventions, including medical, surgical, diagnostic, pharmacologic, devices, rehabilitation, home care, nursing home care, institutional care, and prosthesis. Indirect costs are associated with loss of productivity due to the disease, disorder, or condition suffered by the patient or relatives. A third category, the tangible and intangible losses due to pain and suffering of a patient or relatives often is included in indirect cost studies.

As used herein, “health-related quality of life” is a measure of the impact of the disease, disorder, or condition on an individual's or group of patient's activities of daily living. Preferably, included in pharmacoeconomic studies is an analysis of the health-related quality of life. Standardized surveys or questionnaires for general health-related quality of life or disease, disorder, or condition specific determine the impact the disease, disorder, or condition has on an individuals day to day life activities or specific activities that are affected by a particular disease, disorder, or condition.

As used herein, the term “stratification” refers to the creation of a distinction between patients on the basis of a characteristic or characteristics of the patient. Generally, in the context of clinical trials, the distinction is used to distinguish responses or effects in different sets of patients distinguished according to the stratification parameters. For the present invention, stratification preferably includes distinction of patient groups based on the presence or absence of particular variance or variances in one or more genes. The stratification may be performed only in the course of analysis or may be used in creation of distinct groups or in other ways.

By “drug efficacy” is meant the determination of an appropriate drug, drug dosage, administration schedule, and prediction of therapeutic utility.

By “allele load” is meant the relative ratio of identified gene alleles in the patient's chromosomal DNA.

By “identified allele” is meant a particular gene isoform that can be distinguished from other identified gene isoforms using the methods of the invention.

By “PCR, PT-PCR, or ligase chain reaction amplification” is meant subjecting a DNA sample to a Polymerase Chain Reaction step or ligase-mediated chain reaction step, or RNA to a RT-PCR step, such that, in the presence of appropriately designed primers, a nucleic acid fragment is synthesized or fails to be synthesized and thereby reveals the allele status of a patient. The nucleic acid may be further analyzed by DNA sequencing using techniques known in the art.

By “gene allele status” is meant a determination of the relative ratio of wild type identified alleles compared to an allelic variant that may encode a gene product of reduced catalytic activity. This may be accomplished by nucleic acid sequencing, RT-PCR, PCR, examination of the identified gene translated protein, a determination of the identified protein activity, or by other methods available to those skilled in the art.

By “treatment protocol” is meant a therapy plan for a patient using genetic and diagnostic data, including the patient's diagnosis and genotype. The protocol enhances therapeutic options and clarifies prognoses. The treatment protocol may include an indication of whether or not the patient is likely to respond positively to a candidate therapeutic intervention that is known to affect physiologic function. The treatment protocol may also include an indication of appropriate drug dose, recovery time, age of disease onset, rehabilitation time, symptomology of attacks, and risk for future disease. A treatment protocol, including any of the above aspects, may also be formulated for asymptomatic and healthy subjects in order to forecast future disease risks an determine what preventive therapies should be considered or invoked in order to lessen these disease risks. The treatment protocol may include the use of a computer software program to analyze patient data.

By “patient at risk for a disease” or “patient with high risk for a disease” is meant a patient identified or diagnosed as having drug-induced disease, disorder, dysfunction or having a genetic predisposition or risk for acquiring drug-induced disease, disorder or dysfunction, where the predisposition or risk is higher than average for the general population or is sufficiently higher than for other individuals as to be clinically relevant. Such risk can be evaluated, for example, using the methods of the invention and techniques available to those skilled in the art.

By “converting” is meant compiling genotype determinations to predict either prognosis, drug efficacy, or suitability of the patient for participating in clinical trials of a candidate therapeutic intervention with known propensity of drug-induced disease, disorder or dysfunction. For example, the genotype may be compiled with other patient parameters such as age, sex, disease diagnosis, and known allelic frequency of a representative control population. The converting step may provide a determination of the statistical probability of the patient having a particular disease risk, drug response, or patient outcome.

By “prediction of patient outcome” is meant a forecast of the patient's likely health status. This may include a prediction of the patient's response to therapy, rehabilitation time, recovery time, cure rate, rate of disease progression, predisposition for future disease, or risk of having relapse.

By “therapy for the treatment of a disease” is meant any pharmacological agent or drug with the property of healing, curing, or ameliorating any symptom or disease mechanism associated with drug-induced disease, disorder or dysfunction.

By “responder population” is meant a patient or patients that respond favorably to a given therapy.

In another aspect, the invention provides a method for determining whether there is a genetic component to intersubject variation in a surrogate treatment response. The method involves administering the treatment to a group of related (preferably normal) subjects and a group of unrelated (preferably normal) subjects, measuring a surrogate pharmacodynamic or pharmacokinetic drug response variable in the subjects, performing a statistical test measuring the variation in response in the group of related subjects and, separately in the group of unrelated subjects, comparing the magnitude or pattern of variation in response or both between the groups to determine if the responses of the groups are different, using a predetermined statistical measure of difference. A difference in response between the groups is indicative that there is a genetic component to intersubject variation in the surrogate treatment response.

In preferred embodiments, the size of the related and unrelated groups is set in order to achieve a predetermined degree of statistical power.

In another aspect, the invention provides a method for evaluating the combined contribution of two or more variances to a surrogate drug response phenotype in subjects (preferably normal subjects) by a. genotyping a set of unrelated subjects participating in a clinical trial or study, e.g., a Phase I trial, of a compound. The genotyping is for two or more variances (which can be a haplotype), thereby identifying subjects with specific genotypes, where the two or more specific genotypes define two or more genotype-defined groups. A drug is administered to subjects with two or more of said specific genotypes, and a surrogate pharmacodynamic or pharmacokinetic drug response variable is measured in the subjects. A statistical test or tests is performed to measure response in the groups separately, where the statistical tests provide a measurement of variation in response with each group. The magnitude or pattern of variation in response or both is compared between the groups to determine if the groups are different using a predetermined statistical measure of difference.

In preferred embodiments, the specific genotypes are homozygous genotypes for two variances. In preferred embodiments, the comparison is between groups of subjects differing in three or more variances, e.g., 3, 4, 5, 6, or even more variances.

In another aspect, the invention provides a method for providing contract research services to clients (preferably in the pharmaceutical and biotechnology industries), by enrolling subjects (e.g., normal and/or patient subjects) in a clinical drug trial or study unit (preferably a Phase I drug trial or study unit) for the purpose of genotyping the subjects in order to assess the contribution of genetic variation to variation in drug response, genotyping the subjects to determine the status of one or more variances in the subjects, administering a compound to the subjects and measuring a surrogate drug response variable, comparing responses between two or more genotype-defined groups of subjects to determine whether there is a genetic component to the interperson variability in response to said compound; and reporting the results of the Phase I drug trial to a contracting entity. Clearly, intermediate results, e.g., response data and/or statistical analysis of response or variation in response can also be reported.

In preferred embodiments, at least some of the subjects have disclosed that they are related to each other and the genetic analysis includes comparison of groups of related individuals. To encourage participation of sufficient numbers of related individuals, it can be advantageous to offer or provide compensation to one or more of the related individuals based on the number of subjects related to them who participate in the clinical trial, or on whether at least a minimum number of related subjects participate, e.g., at least 3, 5, 10, 20, or more.

In a related aspect, the invention provides a method for recruiting a clinical trial population for studies of the influence of genetic variation on drug response, by soliciting subjects to participate in the clinical trial, obtaining consent of each of a set of subjects for participation in the clinical trial, obtaining additional related subjects for participation in the clinical trial by compensating one or more of the related subjects for participation of their related subjects at a level based on the number of related subjects participating or based on participation of at least a minimum specified number of related subjects, e.g., at minimum levels as specified in the preceding aspect.

In addition to application of the present invention to drug-induced diseases and conditions, the present invention also provides for the use of variances in genes and gene pathways involved in drug absorption, distribution, metabolism, or excretion (e.g., as specified in any of Tables 1, 3, and 4 herein) of a drug. Thus, the above aspects can be utilized in connection with virtually any type of drug. For example, the pharmacogenetic effect, and the determination of such effect, of variances in genes in pathways involved in drug absorption, distribution, metabolism, or excretion can be utilized, for example, for in connection with drugs and drug classes as described Stanton, International Application No. PCT/US00/01392, filed Jan. 20, 2000, entitled GENE SEQUENCE VARIATIONS WITH UTILITY IN DETERMINING THE TREATMENT OF DISEASE. Further, the particular drug and/or pharmacogenetic determination can also be applied in the context of any disease, disorder, or dysfunction for which a drug treatment is considered or tested, e.g., any of the diseases, disorders, or conditions pointed out in Stanton (Id.). Still further, such analysis and use of pharmacogenetic information for genes involved in drug adsorption, distribution, metabolism, and excretion can also be combined with any of the different aspects described for genes involved in treatment response for other diseases, conditions, and dysfunctions as described in Stanton (Id.).

The use of variance information for genes involved in drug adsorption, distribution, metabolism, and excretion for any drug is advantageous, as those processes can affect the efficacy of any drug. Therefore, variances in such genes that alter one or more of those parameters can be significant in determining interpatient variation in treatment response. Additional aspects and embodiments as described in Stanton, International Application No. PCT/US00/01392, filed Jan. 20, 2000, entitled GENE SEQUENCE VARIATIONS WITH UTILITY IN DETERMINING THE TREATMENT OF DISEASE, are also included in the scope of this invention.

By “pathway” or “gene pathway” is meant the group of biologically relevant genes involved in a pharmacodynamic or pharmacokinetic mechanism of drug, agent, or candidate therapeutic intervention. These mechanisms may further include any physiologic effect the drug or candidate therapeutic intervention renders. Included in this are “biochemical pathways” which is used in its usual sense to refer to a series of related biochemical processes (and the corresponding genes and gene products) involved in carrying out a reaction or series of reactions. Generally in a cell, a pathway performs a significant process in the cell.

By “pharmacological activity” used herein is meant a biochemical or physiological effect of drugs, compounds, agents, or candidate therapeutic interventions upon administration and the mechanism of action of that effect.

The pharmacological activity is then determined by interactions of drugs, compounds, agents, or candidate therapeutic interventions, or their mechanism of action, on their target proteins or macromolecular components. By “agonist” or “mimetic” or “activators” is meant a drug, agent, or compound that activate physiologic components and mimic the effects of endogenous regulatory compounds. By “antagonists”, “blockers” or “inhibitors” is meant drugs, agents, or compounds that bind to physiologic components and do not mimic endogenous regulatory compounds, or interfere with the action of endogenous regulatory compounds at physiologic components. These inhibitory compounds do not have intrinsic regulatory activity, but prevent the action of agonists. By “partial agonist” or “partial antagonist” is meant an agonist or antagonist, respectively, with limited or partial activity. By “negative agonist” or “inverse antagonists” is meant that a drug, compound, or agent that can interact with a physiologic target protein or macromolecular component and stabilizes the protein or component such that agonist-dependent conformational changes of the component do not occur and agonist mediated mechanism of physiological action is prevented. By “modulators” or “factors” is meant a drug, agent, or compound that interacts with a target protein or macromolecular component and modifies the physiological effect of an agonist.

As used herein the term “chemical class” refers to a group of compounds that share a common chemical scaffold but which differ in respect to the substituent groups linked to the scaffold. Examples of chemical classes of drugs include, for example, phenothiazines, piperidines, benzodiazepines and aminoglycosides. Members of the phenothiazine class include, for example, compounds such as chlorpromazine hydrochloride, mesoridazine besylate, thioridazine hydrochloride, acetophenazine maleate trifluoperazine hydrochloride and others, all of which share a phenothiazine backbone. Members of the piperidine class include, for example, compounds such as meperidine, diphenoxylate and loperamide, as well as phenylpiperidines such as fentanyl, sufentanil and alfentanil, all of which share the piperidine backbone. Chemical classes and their members are recognized by those skilled in the art of medicinal chemistry.

As used herein the term “surrogate marker” refers to a biological or clinical parameter that is measured in place of the biologically definitive or clinically most meaningful parameter. In comparison to definitive markers, surrogate markers are generally either more convenient, less expensive, provide earlier information or provide pharmacological or physiological information not directly obtainable with definitive markers. Examples of surrogate biological parameters: (i) testing erythrocyte membrane acetylcholinesterase levels in subjects treated with an acetylcholinesterase inhibitor intended for use in Alzheimer's disease patients (where inhibition of brain acetylcholinesterase would be the definitive biological parameter); (ii) measuring levels of CD4 positive lymphocytes as a surrogate marker for response to a treatment for acquired immune deficiency syndrome (AIDS). Examples of surrogate clinical parameters: (i) performing a psychometric test on normal subjects treated for a short period of time with a candidate Alzheimer's compound in order to determine if there is a measurable effect on cognitive function. The definitive clinical test would entail measuring cognitive function in a clinical trial in Alzheimer's disease patients. (ii) Measuring blood pressure as a surrogate marker for myocardial infarction. The measurement of a surrogate marker or parameter may be an endpoint in a clinical study or clinical trial, hence “surrogate endpoint”.

As used herein the term “related” when used with respect to human subjects indicates that the subjects are known to share a common line of descent; that is, the subjects have a known ancestor in common. Examples of preferred related subjects include sibs (brothers and sisters), parents, grandparents, children, grandchildren, aunts, uncles, cousins, second cousins and third cousins. Subjects less closely related than third cousins are not sufficiently related to be useful as “related” subjects for the methods of this invention, even if they share a known ancestor, unless some related individuals that lie between the distantly related subjects are also included. Thus, for a group of related individuals, each subject shares a known ancestor within three generations or less with at least one other subject in the group, and preferably with all other subjects in the group or has at least that degree of consanguinity due to multiple known common ancestors. More preferably, subjects share a common ancestor within two generations or less, or otherwise have equivalent level of consanguinity. Conversely, as used herein the term “unrelated”, when used in respect to human subjects, refers to subjects who do not share a known ancestor within 3 generations or less, or otherwise have known relatedness at that degree.

As used herein the term “pedigree” refers to a group of related individuals, usually comprising at least two generations, such as parents and their children, but often comprising three generations (that is, including grandparents or grandchildren as well). The relation between all the subjects in the pedigree is known and can be represented in a genealogical chart.

As used herein the term “hybridization”, when used with respect to DNA fragments or polynucleotides encompasses methods including both natural polynucleotides, non-natural polynucleotides or a combination of both. Natural polynucleotides are those that are polymers of the four natural deoxynucleotides (deoxyadenosine triphosphate [dA], deoxycytosine triphosphate [dC], deoxyguanine triphosphate [dG] or deoxythymidine triphosphate [dT], usually designated simply thymidine triphosphate [T]) or polymers of the four natural ribonucleotides (adenosine triphosphate [A], cytosine triphosphate [C], guanine triphosphate [G] or uridine triphosphate [U]). Non-natural polynucleotides are made up in part or entirely of nucleotides that are not natural nucleotides; that is, they have one or more modifications. Also included among non-natural polynucleotides are molecules related to nucleic acids, such as peptide nucleic acid [PNA]). Non-natural polynucleotides may be polymers of non-natural nucleotides, polymers of natural and non-natural nucleotides (in which there is at least one non-natural nucleotide), or otherwise modified polynucleotides. Non-natural polynucleotides may be useful because their hybridization properties differ from those of natural polynucleotides. As used herein the term “complementary”, when used in respect to DNA fragments, refers to the base pairing rules established by Watson and Crick: A pairs with T or U; G pairs with C. Complementary DNA fragments have sequences that, when aligned in antiparallel orientation, conform to the Watson-Crick base pairing rules at all positions or at all positions except one. As used herein, complementary DNA fragments may be natural polynucleotides, non-natural polynucleotides, or a mixture of natural and non-natural polynucleotides.

As used herein “amplify” when used with respect to DNA refers to a family of methods for increasing the number of copies of a starting DNA fragment. Amplification of DNA is often performed to simplify subsequent determination of DNA sequence, including genotyping or haplotyping. Amplification methods include the polymerase chain reaction (PCR), the ligase chain reaction (LCR) and methods using Q beta replicase, as well as transcription-based amplification systems such as the isothermal amplification procedure known as self-sustained sequence replication (3 SR, developed by T. R. Gingeras and colleagues), strand displacement amplification (SDA, developed by G. T. Walker and colleagues) and the rolling circle amplification method (developed by P. Lizardi and D. Ward).

As used herein “contract research services for a client” refers to a business arrangement wherein a client entity pays for services consisting in part or in whole of work performed using the methods described herein. The client entity may include a commercial or non-profit organization whose primary business is in the pharmaceutical, biotechnology, diagnostics, medical device or contract research organization (CRO) sector, or any combination of those sectors. Services provided to such a client may include any of the methods described herein, particularly including clinical trial services, and especially the services described in the Detailed Description relating to a Pharmacogenetic Phase I Unit. Such services are intended to allow the earliest possible assessment of the contribution of a variance or variances or haplotypes, from one or more genes, to variation in a surrogate marker in humans. The surrogate marker is generally selected to provide information on a biological or clinical response, as defined above.

As used herein, “comparing the magnitude or pattern of variation in response” between two or more groups refers to the use of a statistical procedure or procedures to measure the difference between two different distributions. For example, consider two genotype-defined groups, AA and aa, each homozygous for a different variance or haplotype in a gene believed likely to affect response to a drug. The subjects in each group are subjected to treatment with the drug and a treatment response is measured in each subject (for example a surrogate treatment response). One can then construct two distributions: the distribution of responses in the AA group and the distribution of responses in the aa group. These distributions may be compared in many ways, and the significance of any difference qualified as to its significance (often expressed as a p value), using methods known to those skilled in the art. For example, one can compare the means, medians or modes of the two distributions, or one can compare the variance or standard deviations of the two distributions. Or, if the form of the distributions is not known, one can use nonparametric statistical tests to test whether the distributions are different, and whether the difference is significant at a specified level (for example, the p<0.05 level, meaning that, by chance, the distributions would differ to the degree measured less than one in 20 similar experiments). The types of comparisons described are similar to the analysis of heritability in quantitative genetics, and would draw on standard methods from quantitative genetics to measure heritability by comparing data from related subjects.

Another type of comparison that can be usefully made is between related and unrelated groups of subjects. That is, the comparison of two or more distributions is of particular interest when one distribution is drawn from a population of related subjects and the other distribution is drawn from a group of unrelated subjects, both subjected to the same treatment. (The related subjects may consist of small groups of related subjects, each compared only to their relatives.) A comparison of the distribution of a drug response variable (e.g. a surrogate marker) between two such groups may provide information on whether the drug response variable is under genetic control. For example, a narrow distribution in the group(s) of related subjects (compared to the unrelated subjects) would tend to indicate that the measured variable is under genetic control (i.e. the related subjects, on account of their genetic homogeneity, are more similar than the unrelated individuals). The degree to which the distribution was narrower in the related individuals (compared to the unrelated individuals) would be proportionate to the degree of genetic control. The narrowness of the distribution could be quantified by, for example, computing variance or standard deviation. In other cases the shape of the distribution may not be known and nonparametric tests may be preferable. Nonparametric tests include methods for comparing medians such as the sign test, the slippage test, or the rank correlation coefficient (the nonparametric equivalent of the ordinary correlation coefficient). Pearson's Chi square test for comparing an observed set of frequencies with an expected set of frequencies can also be useful.

The present invention provides a number of advantages. For example, the methods described herein allow for use of a determination of a patient's genotype for the timely administration of the most suitable therapy for that particular patient. The methods of this invention provide a basis for successfully developing and obtaining regulatory approval for a compound even though efficacy or safety of the compound in an unstratified population is not adequate to justify approval. From the point of view of a pharmaceutical or biotechnology company, the information obtained in pharmacogenetic studies of the type described herein could be the basis of a marketing campaign for a drug. For example, a marketing campaign that emphasized the superior efficacy or safety of a compound in a genotype or haplotype restricted patient population, compared to a similar or competing compound used in an undifferentiated population of all patients with the disease. In this respect a marketing campaign could promote the use of a compound in a genetically defined subpopulation, even though the compound was not intrinsically superior to competing compounds when used in the undifferentiated population with the target disease. In fact even a compound with an inferior profile of action in the undifferentiated disease population could become superior when coupled with the appropriate pharmacogenetic test.

By “comprising” is meant including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Other features and advantages of the invention will be apparent from the following description of the preferred embodiments thereof, and from the claims.

### DETAILED DESCRIPTION OF THE PREFERRED EMBODIMENTS

First, the content of tables provided in this description is briefly described.

Table 1, the ADME/Toxicology Gene Table, lists genes that may be involved in pharmacological responses involving adsorption, distribution, metabolism, excretion affecting efficacy or safety of drug response. The table has seven columns. Column 1, headed “Class” provides broad groupings of genes relevant to the pharmacology of absorption, distribution, metabolism, or excretion of drugs. The categories are: adsorption and distribution, Phase I drug metabolism, Phase II drug metabolism, excretion, oxidative stress, and immune response. Column 2, headed “Pathway”, provides a more detailed categorization of the different classes of genes by indicating the overall purpose of large groups of genes. These pathways contain genes implicated in the etiology or treatment response of the various patient outcomes detailed in Table 2. Column 3, headed “Function”, further categorizes the pathways listed in column 2.

Column 4, headed “Name”, lists the genes belonging to the class, pathway and function shown to the left (in columns 1-3). The gene names given are generally those used in the OMIM database or in GenBank, however one skilled in the art will recognize that many genes have more than one name, and that it is a straightforward task to identify synonymous names. For example, many alternate gene names are provided in the OMIM record for a gene.

In column 5, headed “OMIM”, the Online Mendelian Inheritance in Man (OMIM) record number is listed for each gene in column 4. This record number can be entered next to the words: “Enter one or more search keywords:” at the OMIM world wide web site. The url is: http://www3.ncbi.nlm.nih.gov/Omim/searchomim.html. An OMIM record exists for most characterized human genes. The record often has useful information on the chromosome location, function, alleles, and human diseases or disorders associated with each gene.

Column 6, headed “GID”, provides the GenBank identification number (hence GID) of a genomic, cDNA, or partial sequence of the gene named in column 4. Usually the GID provides the record of a cDNA sequence. Many genes have multiple Genbank accession numbers, representing different versions of a sequence 5 obtained by different research groups, or corrected or updated versions of a sequence. As with the gene name, one skilled in the art will recognize that alternative GenBank records related to the named record can be obtained easily. All other GenBank records listing sequences that are alternate versions of the sequences named in the table are equally suitable for the inventions described in this application. (One straightforward way to obtain additional GenBank records for a gene is on the internet. General instructions can be found at the NCBI web site at: http://www3.ncbi.nlm.nih.gov. More specifically, the GenBank record number in column 6 can be entered at the url: http://www3.ncbi.nlm.nih.gov/Entrez/nucleotide.html. Once the GenBank record has been retrieved one can click on the “nucleotide neighbors” link and additional GenBank records from the same gene will be listed.

Column 7, headed “locus”, provides the chromosome location of the gene listed on the same row. The chromosome location helps confirm the identity of the named gene if there is any ambiguity.

Table 2 is a matrix showing the intersection of genes and patient outcomes—that is, which categories of genes are most likely to account for interpatient variation in response to treatments. Column 1 is similar to the ‘Class’ column in Table 1, while column 2 is a combination of the ‘Pathway’ and ‘Function’ columns in Table 1. It is intended that the summary terms listed in columns 1 and 2 be read as referring to all the genes in the corresponding sections of Table 1. The remaining columns in Table 2 lists potential effects on efficacy or on eight patient outcomes. The information in the Table lies in the shaded boxes at the intersection of various ‘Pathways” (the rows) and the patient outcomes (the nine columns) An intersection box is shaded when a row corresponding to a particular pathway (and by extension all the genes listed in that pathway in Table 1) intersects a column for a specific effect on patient outcome in response to a candidate therapeutic intervention such that the pathway and genes are of possible use in explaining interpatient differences in response (patient outcomes) to candidate therapeutic interventions. Thus the Table enables one skilled in the art to identify therapeutically relevant genes in patients with one of the nine patient outcomes for the purposes of stratification of these patients based upon genotype and subsequent correlation of genotype with drug response. The shaded intersections indicate preferred sets of genes for understanding the basis of interpatient variation in response to therapy of the indicated disease indication, and in that respect are exemplary. Any of the genes in the table may account for interpatient variation in response to treatments for any of the diseases listed. Thus, the shaded boxes indicate the gene pathways that one skilled in the art would first investigate in trying to understand interpatient variation in response to a candidate therapeutic indications with the listed patient outcomes.

Table 3 lists DNA sequence variances in genes relevant to the methods described in the present invention. These variances were identified by the inventors in studies of selected genes listed in Table 1, and are provided here as useful for the methods of the present invention. The variances in Table 3 were discovered by one or more of the methods described below in the Detailed Description or Examples. Table 3 has eight columns. Column 1, the “Name” column, contains the Human Genome Organization (HUGO) identifier for the gene. Column 2, the “GID” column provides the GenBank accession number of a genomic, cDNA, or partial sequence of a particular gene. Column 3, the “OMIM_ID” column contains the record number corresponding to the Online Mendelian Inheritance in Man database for the gene provided in columns 1 and 2. This record number can be entered at the world wide web site http://www3.ncbi.nlm.nih.gov/Omim/searchomim.html to search the OMIM record on the gene. Column 4, the VGX_Symbol column, provides an internal identifier for the gene. Column 5, the “Description” column provides a descriptive name for the gene, when available. Column 6, the “Variance_Start” column provides the nucleotide location of a variance with respect to the first listed nucleotide in the GenBank accession number provided in column 2. That is, the first nucleotide of the GenBank accession is counted as nucleotide 1 and the variant nucleotide is numbered accordingly. Column 7, the “variance” column provides the nucleotide location of a variance with respect to an ATG codon believed to be the authentic ATG start codon of the gene, where the A of ATG is numbered as one (1) and the immediately preceding nucleotide is numbered as minus one (−1). This reading frame is important because it allows the potential consequence of the variant nucleotide to be interpreted in the context of the gene anatomy (5′ untranslated region, protein coding sequence, 3′ untranslated region). Column 7 also provides the identity of the two variant nucleotides at the indicated position. For example, in the first entry in Table 3, DG90040, the variance is 191G>A, indicating the presence of a G or an A at nucleotide 232 of GenBank sequence DG90040. Column 8, the “CDS_Context” column indicates whether the variance is in a coding region but silent (S); in a coding region and results in an amino acid change (e.g., R347C, where the letters are one letter amino acid abbreviations and the number is the amino acid residue in the encoded amino acid sequence which is changed); in a sequence 5′ to the coding region (5); or in a sequence 3′ to the coding region (3). As indicated above, interpreting the location of the variance in the gene depends on the correct assignment of the initial ATG of the encoded protein (the translation start site). It should be recognized that assignment of the correct ATG may occasionally be incorrect in GenBank, but that one skilled in the art will know how to carry out experiments to definitively identify the correct translation initiation codon (which is not always an ATG). In the event of any potential question concerning the proper identification of a gene or part of a gene, due for example, to an error in recording an identifier or the absence of one or more of the identifiers, the priority for use to resolve the ambiguity is GenBank accession number, OMIM identification number, HUGO identifier, common name identifier.

If a haplotype for any of the genes listed in this table has been identified, a series of nucleotides (A, C, G, T) are listed separated by commas and to the left of each listing is the associated nucleotide location also separated by commas in brackets. For example, if the haplotype listing is T,G,C,A [12, 245, 385, 612] there is a T at position 12, a G at position 246, a C at position 385, and an A at position 612. Below this list will occur the identified variance start, variance, and CDS context for the identified single nucleotide polymorphisms as described above.

Table 4 lists additional DNA sequence variances (in addition to those in Table 3) in genes relevant to the methods of the present invention (i.e. selected genes from Table 1). These variances were identified by various research groups and published in the scientific literature over the past 20 years. The inventors realized that these variances may be useful for understanding interpatient variation in response to treatment of the diseases listed in Table 2, and more generally useful for the methods of the present invention. The columns of Table 4 are similar to those of Table 3, and therefore the descriptions of the rows and columns in Table 3 (above) pertain to Table 4, as do the other remarks.

I. Pharmacokinetic Parameters and Effects on Efficacy

The pharmacokinetic parameters with potential effects on efficacy are absorption, distribution, metabolism, and excretion. These parameters affect efficacy broadly by modulating the availability of a compound at the site(s) of action. Interpatient variation in the availability of a compound drug, agent, or candidate therapeutic intervention can result in a reduction of the available compound or more compound at the site of action with a corresponding altered clinical effect. Differences in these parameters, therefore, can be a potential foundation of interpatient variability to drug response.

A. Pharmacokinetic Parameters that Result in a Reduction of Available Drug

1. Absorption—Depending on the solubility of the drug, and its ability to passively cross membranes is fundamental to the ability of the drug, agent, or candidate therapeutic intervention to effectively enter the circulation and gain access to the principle site of action. For enteral delivery or administration, absorption is a critical first step in the pharmacologic process. Within the gastrointestinal tract, absorption of a drug, agent, or candidate therapeutic intervention can be affected by the pH of the contents, speed of gastric emptying, and presence of chelating or binding molecules to the drug, agent or candidate therapeutic intervention. Each of these parameters can effectively reduce the rate of passive absorption of the drug across the gastrointestinal mucosal membrane.

2. Distribution—Once absorbed, the drug, agent or candidate therapeutic intervention must be delivered or distributed to the primary site of pharmacologic action. Although distribution is dependent on regional blood flow and cardiac output; distribution may be further affected by the rate and extent of sequestration of the drug into biological spaces that render the product unavailable to the principle or primary site of pharmacologic site of action. For example, many drugs are actively transported into biological compartments. These processes, if over- or under active may affect the availability and hence reduce the efficacy of the product. Further, only unbound drug may be effective to a cell, tissue, or physiological process, and bound product may be transported to a space that is physiologically unrelated to the pharmacologic mechanism of action or may be of deleterious adverse or toxic consequence.

3. Metabolism—Induction of metabolic enzymes to covalently modify the parent drug, agent or candidate therapeutic intervention may reduce the ability of the parent drug to elicit a pharmacologic action. Metabolism may affect the target active site binding, rate and extent of distribution and excretion, and overall availability of the active molecule.

4. Excretion—If the excretion of the drug or drug metabolite is rapid, less drug is available to elicit a pharmacologic effect.

B. Pharmacokinetic Parameters that Result in More Available Drug

1. Absorption—Enhanced absorption of drugs, agents or candidate therapeutic interventions may result in increased drug availability. For example, in some cases of decreased gastric emptying, there is an enhanced degree of absorption by prolonging contact with gastrointestinal mucosal membranes. In others, a change in the solubility of the drug may enhance the passive transport across the gastrointestinal mucosal membrane.

2. Distribution—Since free drug is the form that renders pharmacologic action and is metabolized and excreted, drug binding may serve to protect the drug from mechanisms of inactivation. The rate and extent of drug binding affects the free drug concentration relative to the total concentration.

3. Metabolism—If drug metabolism induction is occurring and the inducer is rapidly removed without adjustment in the dose of the drug, drug metabolism may be decreased and adverse effects or toxicities may occur.

4. Excretion—If inhibition of active transport of the parent drug or metabolite across the bile cannicula or the renal tubule, there is a net result of enhanced drug availability.

II. Impaired Drug Tolerability and Drug-Induced Disease, Disorder, Dysfunction or Toxicity

In response to chemical substances, drugs, or xenobiotics, drug-induced disease, disorder, dysfunction, or toxicity manifests as cellular damage or organ physiologic dysfunction, with one potentially leading to the other.

Adverse drug reactions can be categorized as 1) mechanism based reactions which are exaggerations of pharmacologic effects and 2) idiosyncratic, unpredictable effects unrelated to the primary pharmacologic action. Although some side effects appear shortly after administration of a drug, some side effects appear long after drug administration or after cessation of the drug. Furthermore, these reactions can be categorized by reversible or irreversible manifestations of the drug-induced toxicity referring to whether the clinical symptomology subsides or persists upon withdrawal of the offending agent.

In the first category, excessive drug effects may result from alterations of pharmacokinetic parameters by either drug-drug interactions, pathophysiologic disease mediated alterations in the organs or processes involved in absorption, distribution, metabolism, or excretion, or genetic predisposition to heightened pharmacodynamic effect of the drug. The excessive or heightened response may be receptor or drug target or non-receptor or non-drug target mediated.

There are a large number of adverse events that are suspected and or known to occur as a result of administration of a drug, agent, or candidate therapeutic intervention. For example, many antineoplastic agents act by prevention of cell division in dividing cells or promoting cytotoxicity via disruption of DNA synthesis, transcription, and formation of mitotic spindles. These agents, unfortunately, do not distinguish between normal and cancerous cells, e.g. normally dividing cells and cancer cells are equally killed. Therefore, adverse events of antineoplastic agents include bone marrow suppression leading to anemia, leukopenia, and thrombocytopenia; immunosuppression rendering the patient susceptible and vulnerable to infectious agents; and initiation of mutagenesis and the formation of alternate forms of cancer, in many cases, acute myeloid leukemia.

In another example of predictable adverse events related to drug therapy is immunosuppression as a result of therapy to reduce or ablate immune response. This therapy includes but is not exclusive to prevention of graft vs. host or autoimmune disease. These agents, e.g. corticosteroids, cyclosporine, and azathioprine, all suppress humoral or cell-mediated immunity. Patients taking these agents are rendered susceptible to microbial infections, particular opportunistic infections such as cytomegalovirus, pneumocystis carnii, Candida, and sperigillus. Furthermore, long-term immunosuppressive therapy is associated with increased risk of developing lymphoma. Individual drugs are associated with renal injury (cyclosporine) and interstitial pneumonitis (azathioprine).

In the second category of adverse events, idiosyncratic reactions arise often by unpredictable, unknown mechanisms or reactions that evoke immunologic reactions or unanticipated cytotoxicity.

Adverse reactions in this category are often found together, because often it is difficult to ascertain the etiology of the offending reaction. These toxic events can be specific for a target organ, e.g. ototoxicity, nephrotoxicity, hepatotoxicity, neurotoxicity, etc. or are caused by reactive metabolic intermediates and are toxic or create local damage usually near the site of metabolism.

Immunologic reactions to drugs are thought or result from the combination of the drug or agent with a protein to form an antigenic protein-drug complex that stimulates the immune system response. Without the formation of a complex, most small molecular drugs are unable, alone, to elicit an immunological response. First exposure to the offending drug produces a latent reaction, subsequent exposures usually results in heightened and rapid immunological response. These allergic reactions, characterized by immunohypersensitivity, are most dramatic in anaphylaxis. There are other immune responses that result in adverse reactions or toxicities. They include but are not limited to: 1) immune response mediated cytotoxicity which occurs when the drug-protein complex binds to the surface of a cell and this cell-complex is then recognized by circulating antibodies; 2) serum sickness which occurs when immune complexes of drug and antibody are found in the circulation; and 3) lupus syndromes in which the drug or reactive intermediate interact with nuclear material to stimulate the formation of antinuclear antibodies.

In addition to the immune phenomena described above, there are other drug reactions that are syndromes involving allergic reactions. These reactions include, but are not limited to, skin e rashes, drug induced fever, pulmonary reactions, hepatocellular or cholestatic reactions, interstitial nephritis, and lymphadenopathy. Further, there are some drug reactions that mimic allergic reactions but are not immune related. For example, such reactions are due to direct release of mediators by drugs and are called anaphylactoid reactions. An example of this type of adverse event is reaction to radiocontrast dye.

These are common adverse drug reactions that may prevent a candidate therapeutic intervention from use, continued development, and marketing rights. Some of these reactions are reversible, others are not.

Adverse drug reactions include, but are not limited to, the following organs systems: a) hemostasis which encompass blood dyscrasias (feature of over half of all drug-related deaths) which are bone marrow aplasia, granulocytopenia, aplastic anemia, leukopenia, pancytopenia, lymphoid hyperplasia, hemolytic anemia, and thrombocytopenia; b) cutaneous which encompass urticaria, macules, papules, angioedema, morbilliform-maculopapular rash, toxic epidermal necrolysis, erythema multiforme, erythema nodosum, contact dermititis, vesicles, petechiae, exfolliative dermititis, fixed drug eruptions, and severe skin rash (Stevens-Johnson syndrome); c) cardiovascular which includes arrythmias, QT prolongation, cardiomyopathy, hypotension, or hypertension; d) renal which includes glomerulonephritis and tubular necrosis; e) pulmonary which includes asthma, acute pneumonitis, eosinophilic pneumonitis, fibrotic and pleural reactions, and interstitial fibrosis; f) hepatic which includes steatosis, hepatocellular damage and cholestasis; g) systemic which includes anaphylaxis, vasiculitis, fever, lupus erythematosus syndrome; and h) the central nervous system which includes tinnitus and dizziness, acute dystonic reactions, parkinsonian syndrome, coma, convulsions, depression and psychosis, and respiratory depression.

In the cases whereby severe, fatal reactions occur after drug administration, there may be a warning label in the product insert.

For example, tricyclic antidepressants can cause central nervous system depression, seizures, respiratory arrest, cardiac arrythmias and arrest. The mechanism for the injury is a result of the increased synaptic concentrations of biogenic amines and inhibition of postsynaptic receptors.

Acetominophen can cause hepatic necrosis as a result of prolonged high dose usage or overdose. In the hepatocyte, acetominophen is converted to a toxic metabolite that binds to glutathione. As the concentration of acetominophen increases the levels of glutathione are depleted and the toxic acetominophen metabolite then binds liver macromolecules. Aggregation of polymorphonuclear neutrophils in hepatic microcirculation may cause ischemia and foster necrotic events.

Halothane can cause hepatic necrosis as well as prodrome fever and jaundice. Interestingly, the liver effects of halothane are usually after a first time exposure. The hepatic reaction is thought to occur via a genetic predisposition to deranged metabolism with the formation of toxic metabolites.

III. Pharmacokinetic Parameters as Potential Mechanisms of Drug-Induced Adverse Reactions Leading to Disease, Disorder, Dysfunction or Toxicities

A. Absorption

Absorption is the pharmacokinetic parameter that describes the rate and extent of the drug, agent, or candidate therapeutic intervention leaves the site of administration. Although absorption is critical for the drug, agent, or candidate therapeutic intervention to ultimately reach the site of physiologic action, the term bioavailability is the parameter that is clinically relevant. Bioavailability is the term used to define the extent to which the active component of the drug, agent, or candidate therapeutic intervention reaches the its site of physiologic action or a biological fluid to which has access to the site of biological action. Although bioavailability is related to all pharmacokinetic parameters, e.g. absorption, distribution, metabolism, and excretion, bioavailability is primarily dependent on the first ability of the drug, agent, or candidate therapeutic intervention to be absorbed from the site of delivery, i.e. cross cellular membranes.

There are many factors that influence absorption of a drug, agent, or candidate therapeutic intervention. For example, compound solubility, conditions of absorption, and route of administration. In the present invention, we concern ourselves with genes that are involved in the active or passive process of drug, agent, or candidate therapeutic intervention absorption through a biological membrane.

The absorption surface is dependent on the route of administration. For example, absorption of drugs can occur via 1) oral (enteral); 2) sublingual; 3) injections (parenteral, i.e., intravenous, intramuscular, intraarterial, intrathecal, intraperiotoneal, or subcutaneous); 4) rectal; 5) inhalation (pulmonary); 6) topical application (skin and eye). In each of these routes of administration, the adsorption rate and extent is dependent on the concentration of the drug at the site, the patency of the epithelial cells, local biological conditions, and function of the active or passive transport.

Absorption can affect both the efficacy and safety of a drug, agent, or candidate therapeutic intervention. For example, for a compound to achieve full pharmacologic potential, it must be available at the target site, be active, and be unbound. In regards to safety, absorption affects safety in one or more of the following: site of delivery pain, necrosis, or irritation; rate of administration; and erratic available concentrations.

B. Distribution

The distribution of the drug, agent, or candidate therapeutic intervention is dependent on the rate and extent the compound enters the bloodstream. Once in the bloodstream, the compound may be distributed to the interstitial and cellular fluids. The distribution of drugs to target tissues can be categorized into two phases. The first distribution phase, is dependent on cardiac output and regional blood flow, both of which are dependent on the health and status of the cardiovascular system. In a second distribution phase, diffusion into tissues is dependent on the level and extent that the drug, agent, or candidate therapeutic intervention is bound. Drug binding by proteins found in the blood can serve to protect the compound from modifications by enzymes, proteins, or compounds in the circulation and or limit the bioavailability of the compound to enter target tissues or individual cells.

Drug entry into tissues requires free drug, and drug binding proteins may limit this active or passive transport. Once distributed into tissues, the drug may be sequestered within that tissue, to render full pharmacologic activity or to prevent that drug from reaching the appropriate target tissue.

Distribution can affect both the efficacy and safety of a drug, agent, or candidate therapeutic intervention. For example, for a compound to achieve full pharmacologic potential, it must be available at the target site, be active, and be unbound. In regards to safety, distribution affects safety in one or more of the following: distribution to a tissue that is more or less affected by the pharmacologic action of the compound, erratic available concentrations, and tissue specific distribution characteristics.

C. Metabolism

Drugs or xenobiotics, are usually found in the circulation bound to plasma proteins, generally but not exclusive to serum albumin. It is the bound form of the drug that is taken up by the hepatocyte. Bile salts in the circulation are taken up via organic anion transporters. Once inside the hepatocyte, the drug or bile salt is a substrate for a series of reactions that are either oxidative or reductive or reactions that are conjugative steps in the metabolism of the substrate. Generally these chemical modifications are a refined process to render the substrate more hydrophilic, or polar, to be more likely excreted in the bile (via the intestinal tract) or urine (via the kidneys). However, there are exceptions whereby the redox reactions produce reactive intermediates or products that retard elimination. Except for their role in detoxification, there is little in common among the enzymes involved in the redox detoxification reactions. For certain enzymes there are specific groups that will act as substrates, for others there are general classes of chemical compounds that will be suitable substrates for a given enzyme or enzymes.

In the mammalian liver these mechanisms to detoxify and/or enhance the excretion of metabolic by-products, endogenous substrates, and exogenous molecules. The ability to determine whether hepatic function if inadequate is based upon clinical observation, e.g., the presence of jaundice, right upper quadrant abdominal discomfort or pain, pruritis, or by clinical laboratory analyses, e.g., aspartate transaminase (AST or SGOT) or alanine transferase (ALT or SGPT). The hepatic metabolic and excretory mechanisms are critical for short- and long-term survival and are inheritable characteristics. These hepatic biotransformations mechanisms have broad substrate specificity that have been evolutionarily inherited for the host protection from environmental, biological, and chemical substances.

There are two categories of drug, agent, or candidate therapeutic intervention biotransformation (metabolism). In the first, phase I, functionalization reactions occur. Phase I reactions introduce or expose a functional group to the parent compound. In general, phase I reactions render the parent compound pharmacologically inactive, however there are examples of phase I reaction activation or retention of activity. In phase II reactions, biosynthetic reactions occur. Phase II conjugation reactions leads to a covalent linkage between a functional group on the parent compound with glucuronic acid, sulfate, glutathione, amino acids, or acetate. The metabolic conversion of drugs is the liver, however, all tissues have enzymatic activity.

Factors affecting drug biotransformation are 1) induction of metabolizing enzymes, 2) inhibition of enzymatic reactions, and 3) genetic polymorphisms. It is the interplay of these factors and the health and well being of the patient or subject that determines the fate of parent drug molecules in the body.

The first factor affecting drug biotransformation is induction of metabolizing enzyme activity. The metabolic processes that modify drugs or chemicals (oxidation, reduction, or conjugation) can be induced to significant enzymatic activity. Under physiological conditions, the induction process is in place to coordinately metabolize excess substrates. The induction process can be both at the level of enzymatic activity and increased protein levels of the pertinent enzyme or enzymes. Induction may include one or several of the enzymatic pathways or processes in response to the presence of drugs, xenobiotics, endogenous substrates, or metabolic by-products. There may or may not be increased toxicity as a result of increased concentrations of metabolites. Further, induction of phase I reactive processes (oxidation or reduction reactions) may or may not induce the phase II reactive processes (conjugation reactions).

The second factor affecting drug biotransformation is the inhibition of metabolic enzymes. Enzymatic inhibition can occur via 1) competition of two or more substrates for the enzymatic active site, 2) suicide inhibitors, or 3) depletion of required cofactors for the enzymatic pathways or processes in phase I or phase II reactions.

In competitive inhibition, two or more drugs, xenobiotics, or substrates present can interact with the active site of the enzyme. If one drug binds specifically to the enzymatic active site or to an other intracellular regulatory protein molecule, other compounds are blocked from binding and remain unbound. In this case, unmetabolized parent drug or xenobiotic remains in the circulation, potentially for extended periods of time. Competitive inhibition is dependent on the relative specificity of the substrates for the enzymatic active site and the concentration of the drugs or substrates. An example of competitive drug biotransformation inhibition are cimetidine and ketoconazole which inhibit oxidative drug metabolism by forming a tight complex with the heme iron complex of cytochrome P450, and macrolide antibiotics such as erythromycin and troleandomycin are metabolized to products bind to heme groups on the cytochrome P450 molecules.

In the second case, the inhibition of enzymes involved in the drug biotransformation process may also occur by suicide inactivation. In these cases, the drug or xenobiotic may interact and covalently modify or render inactive the enzyme involved in the metabolic pathway. In this way, the parent drug compound or molecule is not metabolized, nor is it free to interact with another molecule. Examples of suicide inactivators are secobarbital and synthetic steroids (norethindrone or ethinyl estradiol) which bind to cytochrome P450 and destroy the heme portion of the enzyme unit.

In the third case, inhibition of the enzymes involved in the drug biotransformation pathway can also occur by agents or compounds or physiological status that deplete NADPH or other cofactors required for the enzymatic reactions to occur. In the cases of phase I oxidation or reduction, lack of oxygen or NADPH, may reduce the efficiency and activity of a particular enzyme. In phase II reactions, cofactors provide specific groups for the enzymatic covalent modification of the drug or xenobiotic. These phase II cofactors are required for conjugation biotransformation reactions to occur and depletion of these cofactors would be rate limiting.

The third factor that can affect drug biotransformation is genetic polymorphism. Differences among individuals to metabolize drugs have long been known. Observed phenotypic differences, as determined by amount of drug excreted, through polymorphically controlled pathway/s has lead to a generalized classification of slow (poor) metabolizers and fast (rapid or extensive) metabolizers. In general, poor metabolizers are those with impaired metabolism of a drug via a polymorphic pathway have been associated with an increased incidence of adverse effects. In addition, to date all major deficiencies in drug metabolizing activity are inherited as autosomal recessive traits. Fast or rapid metabolizers are those individuals with processes that extensively metabolize a drug via a polymorphic pathway. The fast or rapid metabolizers have been associated with an increased incidence of ineffective treatment. In these individuals active drug is rapidly metabolized to less active or inactive metabolites such that a reassessment of the pharmacokinetic parameters and dosing regimen may require analysis or readjustment, respectively, for appropriate therapy to occur.

The first observed and catalogued genetic polymorphism associated with drug metabolism was described for isoniazid. Isoniazid is a primary drug prescribed for the chemotherapy of tuberculosis. Marked interindividual variation in the elimination of this drug was observed and genetic studies of families revealed that this variation was genetically controlled. Isoniazid is predominantly metabolized via N-acetylation. In the analysis of the phenotypically distinct individuals, it was shown that slow acetylators were homozygous for a recessive gene and fast acetylators were homozygous or heterozygous for the wild type gene. It has been determined that the incidence of the slow acetylator phenotype is approximately 50% for U.S. Caucasians and blacks, 60-70% of Northern Europeans, and 5-10% in Asians. Other drugs have been shown to be polymorphically acetylated, e.g. sulfonamides (sulfadiazine, sulfamethazine, sulfapyridine, sulfameridine, and sulfadoxine), aminoglutethimide, amonafide, amrinone, dapsone, dipyrone, endralazine, hydralazine, prizidilol, and procainamide. Other drugs that first undergo metabolism and then polymorphically acetylated are clonazepam and caffeine.

Another common genetic polymorphism associated with oxidative metabolism is exemplified by the drug debrisoquine (a sympatholytic antihypertensive). It was discovered that variable inter-patient hypotensive response was due to differing metabolic rates of debrisoquine 4-hydroxylase. Further analysis of family studies revealed that oxidative metabolic reactions are under monogeneic control. A cytochrome P450 enzyme, CYP2D6, was determined to be the target gene for debrisoquine 4-hydroxylase activity. Poor metabolizers of desbrisoquine are homozygous for a recessive CYP2D6 allele and rapid or fast metabolizers are homozygous or heterozygous for the wild type CYP2D6 allele. Urinary metabolic ratio can be determined after administration of a probe drug and phenotypic assignments (poor or extensive metabolizer) can be identified. The extent of debrisoquine metabolic analysis achieved clinical importance as it was determined that other drugs were poorly metabolized in individuals that poorly metabolized debrisoquine. For example, anti-arryhthmics such as flecainide, propafenone, and mexiletine; antidepressants such as amitryptiline, clomipramine, desipramine, fluoetine, imipramine, maprotiline, mianserin, paroxetine, and nortriptyline; neuroleptics such as haloperidol, perphenazine, and thioridazine; antianginals such as perhexilene; opioids such as dextromethorphan and codeine; and amphetamines such as methylenedioxymethamphetamine. Further, many β-adrenergic antagonists are metabolized and are subject to polymorphic influence in elimination patterns.

Another example of a genetic polymorphism affecting oxidative metabolism was described for mephenytoin, a drug prescribed for epilepsy. It was shown that a deficiency in the 4′-hydroxylation of S-mephenytoin is inherited as an autosomal recessive trait. The other main metabolic pathway, N-methylation of R-mephenytoin to 5-phenyl-5-ethylhydantoin remains unaffected. Individuals with poor metabolic rate of mephenytoin are subject to adverse central effects, i.e. sedation. Other drugs can be grouped into the poor mephenytoin metabolizers are mephobarbital, hexobarbital, side-chain oxidization of propanolol, the demethylation of imipramine, and the metabolism of diazepam and desmethyldiazepam. Further analysis of other drugs such as the metabolism of antidepressant drugs (citalopram), the proton pump inhibitor omeprazol, the antimalarial drugs pantoprazole and lansoprazole cosegregate with mephenytoin metabolites.

Because the majority of metabolic enzymes for the conduct of drug biotransformation occurs in the liver, impairment of liver function as a result of hepatic pathological conditions or other disease states can lead to alterations of hepatic or other organ metabolic drug biotransformation. Liver disease pathologies such as hepatitis, alcoholic liver disease, fatty liver disease, biliary cirrhosis, and hepatocarcinomas can impair function of normal physiological metabolic pathways. Further, decreases in hepatic circulation as a result of cardiac insufficiency, hypertension, vascular obstruction, or vascular insult can affect the rate and extent of drug biotransformation. For example drugs with a high hepatocyte extraction ratio would have different metabolism rates affected by alterations of hepatic circulation. Changes in liver blood flow can affect the rate and extent of the metabolism and the clearance of the parent drug. In all cases of hepatic pathological conditions, the affect on drug biotransformation and clearance of parent drugs or metabolized products will be dependent on the severity and extent of the liver organ and hepatocellular damage.

Although hepatic damage may affect the metabolism and clearance of a parent drug or metabolic by-product, residual concentrations of parent drug or metabolic by-products may be deleterious to the liver and its metabolic functions. Following nonparenteral (enteral) administration of a drug, a significant portion of the drug will be metabolized by intestinal or hepatic enzymes before it reaches the general circulation. This first pass effect may generate active drug (administered drug was a prodrug), inactive drug, or toxic drug. Prior to circulation of the metabolized product, circulation to the kidney, the major organ for excretion of the hydrophilic moiety, and excretion via the urine will occur. Therefore, a metabolic product of hepatic metabolic pathways can affect the liver, kidney, and other organs of the body prior to excretion.

1. Phase I Drug Biotransformation: Oxidation and Reduction Reactions

Enzymatic Oxidation of Drugs

In oxidative metabolism, oxidases catalyze the transfer of electrons from substrate to oxygen, generating either hydrogen peroxide or superoxide anions. There are two oxidases present in hepatocytes; they are aldehyde oxidases and monoamine oxidases. Both of these enzymes have broad substrate specificity and contribute broadly to the metabolism of drugs. A third oxidase, xanthine oxidase, may contribute to the oxidation of drugs, due its ability to catalyze the oxidation of heterocyclic aromatic amines, for example methotrexate and 6-mercaptopurine. Xanthine oxidase in intact tissues is present as a NAD-dependent dehydrogenase, and is converted to an oxidase when there is disruption of the tissue, for example during hepatic cellular damage.

Aldehyde oxidase catalyzes the oxidation of fatty aldehydes to carboxylic acids and the hydroxylation of substituted pyridines, pyrimidines, purines, and pteridines. Generally, xenobiotic aromatic nitrogen heterocycles are metabolized by this enzyme.

Monoamine oxidase is present in two forms, A and B. They are dimeric proteins consisting of identical subunits and FAD is covalently linked to the protein through a cysteinyl residue. Catalytic cycles of monoamine oxidases A or B occur in discrete steps that take an amine and convert it to an aldehyde, while in the process creating hydrogen peroxide and ammonia. These oxidases have a broad specificity; they protect mitochondrial proteins from xenobiotic amines and hydrazines. Further neurotransmitters are metabolized through this route, e.g. serotonin, dopamine, and catecholamines. Primary alkylamines containing unsubstituted methylene group or groups adjacent to the nitrogen exhibits activity. Activity increases as the length of a side chain, with optimal side length being C6. These enzymes also catalyze the oxidation of secondary and tertiary amines and acyclic amines. Hydrazines can be oxidized by these oxidases. Substrates for monoamine oxidases include but are not exclusive to the following amines: benzylamine, dopamine, tyramine, epinephrine, N-methylbenzylamine, and N,N-dimethlybenzylamine; and the following hydrazines: procarbazine 1,2-dimethylhydrazine.

Mono-oxygenases are present in liver cell homogenates and contain two distinct types of xenobiotic mono-oxygenases. They are the cytochrome P450 and the flavin-dependent mono-oxygenases.

The liver microsomal P-450 system consists of a flavoprotein, and a family of related, but distinct, hemoproteins. The flavoprotein catalyzes the transfer of the electrons from NADPH to the hemoprotein, and is the mono-oxygenase. The reaction also requires phosphatidylcholine. The reductase is a monomeric flavoprotein that contains both FAD and FMN. The reductase is specific for NADPH as a reductant, but other oxidants can be substituted. In addition to cytochrome P-450, the flavoprotein catalyzes reduction of quinones, nitro, and azo compounds.

There are many P450 gene families. Subsequent cloning and sequence determination has afforded the ability to divide this gene family into three main groups, CYP1, CYP2, and CYP3, that are responsible for the majority of drug biotransformation. There are further subdivisions in each of these families, examples being CYP2D6, CYP3A4, CYP2E1, as well as others.

Examples of enzymatic inductive processes that affect biotransformation reactions involve the P450 gene family. Specifically, glucocorticoids and anticonvulsants induce CYP3A4; isoniazid, acetone, and chronic ethanol consumption for CYP2E1. Many inducers of the cytochrome P450 enzymes also induce conjugation metabolic enzymes, e.g. glucuronosyltransferases.

In contrast to the monooxygenases, multiple forms of the terminal oxidase (P-450) are present in the hepatocyte. There are many distinct isoforms characterized in different species including humans. It should be noted that mitochondrial P-450 exhibit little or no activity in the metabolism of drugs, xenobiotics, biological compounds, or chemicals. Representative functional groups oxidated by the microsomal P-450 system are as follows: alkanes (hexane, decane, hexadecane); alkenes (vinyl chloride, aflatoxin-B1, dieldrin); aromatic hydrocarbons (naphthylene, bromobenzene, benzo(a)pyrene, biphenyl); alipathic amines (aminopyrine, benzphetamine, ethylmorphine); heterocyclic amines (3-acetylpyridine, 4,4′-bipyridine, quinoline); amides (N-acetlyaminofluorene, urethane); ethers (indemethacin, pheancetin, p-nitroanisole); and sulfides (chloropromazine, thioanisole).

There are many P450s that have been identified in human liver. Substrate specificities vary among these P-450 dependent mono-oxygenases. For example, P4501A1 prefers polycyclic aromatic hydrocarbons; P-4501A2 prefers arylamines, arylamides; P-450A26 prefers coumarin, 7-ethoxycoumarin; P-450 2C8, 2C9, 2C10 prefers tolbutamide, hexobarbital; P-450 2C18 prefers mephenytoin; P-450 mp-1, mp-2 prefers debrisoquine and related amines; P450 2E1 prefers ethanol, N-nitrosoalkylamines, vinyl monomers; P-450 3A3, 3A4, 3A5, 3A7 prefers dihydropyridines, cyclosporin, lovastatin, aflatoxins.

The effect of genetic polymorphism of the P450s has been known for some time. For example, debrisoquine and related drugs; alfentanil, tolbutamide; (S)mephenytoin. Because the P450s can be induced by xenobiotics, an enhanced metabolic rate or efficiency can lead to one drug affecting the potency, efficacy, dosing of another. For example, women taking rifampicin or barbiturates can lead to metabolic inactivation of synthetic oral contraceptives.

The flavin-containing mono-oxygenases are the principle enzymes catalyzing the N-oxidation of tertiary amine drugs to N-oxides. The N-oxides are found in abundance in serum. Although isoforms have been identified and the catalytic cycle is similar to the cytochrome P450 system, flavin-containing mono-oxygenases substrate specificity differs. Unlike the other flavin-bearing mono-oxygenases, these flavin-containing mono-oxygenases are present in the cell as very reactive oxygen-activated form. It is believed that particular protein structure stabilizes the nucleophilic molecule. Since the molecule is so highly reactive, precise substrate-to-enzyme fit is unnecessary. The following lists substrate types and examples oxidized by the flavin-containing mono-oxygenases: tertiary amines (trifluroperazine, bromopheniramine, morphine, nicotine, pargyline); secondary amines (desipramine, methamphetamine, propanolol); hydrazines (1,1-demethlyhydrazine, N-aminopiperidine, 1-methyl-1-phenylhydrazine); thiols and disulfides (dithiothreitol, β-mercaptomethanol, thiophenol); thiocarbamides (thiourea, methimazole, propylthiouracil); sulfides (dimethylsulfide, sulindac sulfide).

Examples of drugs that undergo oxidative reactions are: N-dealkylation (imipramine, diazepam, codeine, erythromycin, morphine, tamoxifen, theophylline); O-dealkylation (codeine, indomethacin, dextromethorphan); alipathic hydroxylation (tolbutamide, ibuprofen, pentobarbital, meprobamate, cyclosporin, midazolam); aromatic hydroxylation (phenytoin, phenobarbital, propanolol, phenylbutazone, ethinyl estradiol); N-oxidation (chlorpheniramine, dapsone); S-oxidation (cimetidine, chlorpromazine, thioridazine); deamination (diazepam, amphetamine).

Enzymatic Reduction of Drugs

The reductases are a class of enzymes that are involved in the metabolic reduction of xenobiotics. This class of enzymes includes the aldehyde and ketone reductases, the quinone reductases, the nitro and nitroso reductases, the azoreductases, the N-oxide reductases, and the sulfoxide reductases. These classes of enzymes are involved in sequential one-electron reduction of some functional groups and produce radicals that can produce damage cellular components directly or indirectly.

The dehydrogenases consist of alcohol dehydrogenases, aldehyde dehydrogenases, or dihydrodiol dehydrogenases. This class of enzymes is involved in the catalysis of hydrogen transfer to a hydrogen acceptor, usually a pyridine nucleotide.

Hydrolysis of Drugs

Alternative reactions of detoxification and metabolism of drugs and xenobiotics are initial steps of hydrolysis. Esters, amides, imides, or other functional groups that are generated as a result of a hydrolysis reaction can alter the hydophilicity of a molecule and enhance urinary excretion. Hydrolysis occurs both enzymatically and nonenzymatically. Hydrolysis of proteins before they are degraded has been suggested as a step in the process of the aging of intracellular proteins. Antibodies with an affinity for certain esters and certain proteases e.g. 3-phosphoglyceraldehyde dehydrogenase and carbonic anhydrase, have been shown to have esterase activity.

Enzymatic hydrolysis of drugs and xenobiotics include the following enzymes: esterases, amidases, imidases, and epoxide hydratases. Examples of drugs undergoing hydrolysis reactions are: procaine, aspirin, clofibrate, lidocaine, procainamide, indomethacin.

Other hydrolytic processes include reactions owing to both enzymes in tissues, circulation, and those elaborated by microorganisms in the lower bowel; for example, sulfatases, glucoronidases, and phosphatases.

2. Phase II Drug Biotransformation: Conjugation Reactions

In addition, to the redox reactions of the hepatocyte to detoxify or metabolize xenobiotics, there are a series of conjugation reactions. The substrates for these reactions are generally the products from the redox reactions described above. These conjugation reactions involve donation of a suitable hydrophilic molecular group to an accepting xenobiotic or its metabolite. The major function of these covalent modifications is to render the parent compound pharmacologically inactive. The covalent addition of such a group to a parent drug or compound not only inactivates the substrate but also renders the recipient molecule more polar and is more readily excreted via the bile ducts into the intestinal tract or via the urine.

Lipophilic compounds that have one of the functional groups that can serve as an acceptor undergo enzymatic catalysis with a second, donor substrate. The conjugation reactions include the following broad categories: glucuronidation, sulfation, methylation, N-acetylation, and conjugation with amino acids. The enzymes involved in these reactions are as follows: UDP-glucuronyltransferase, alcohol sulfotransferase, amine N-sulfotransferase, phenol sulfotransferase, glutathione transferase, catechol O-methyltransferase, amine N-methyltransferase, histamine N-methyltransferase, thiol S-methyltransferase, benzoyl-CoA glycine acyltransferase, acetyltransacetylase, cysteine S-conjugate N-acetyltransferase, cysteine S-conjugate N-acetyltransferase, cysteine conjugate β-lyase, thioltransferase, and rhodanese. Each of these enzymes has donor and acceptor specificities. The importance of these reactions in the detoxification and metabolism of drugs and xenobiotics are discussed in the examples

Examples of drugs that are known to be conjugated are: glucuronidation (acetominophen, morphine, diazepam); sulfation (acetominophen, steroids, methyldopa); acetylation (sulfonamides, isoniazid, dapsone, clonazepan).

D. Excretion

Excretion of parent drugs and metabolites can occur in the excretory organs, namely the kidneys, liver, lungs, skin, and breasts (milk). The kidneys are the most important organs for the excretion of drugs and metabolites. Renal excretion involves glomerular filtration, active tubular absorption, and passive tubule reabsorption. The more hydrophilic the compound is the more readily excreted via urine. In addition, many drugs and metabolites are excreted via the bile into the intestinal tract. These metabolites may be excreted in the feces, or may be reabsorbed by the gastrointestinal epithelial cell lining. Organic anions and cations, steroids, fatty acids, and other drugs may be specifically transported into the bile canniculus.

In all of the metabolism and excretion routes, the physiologic goal is to detoxify and rid the body of drugs, xenobiotics, endogenous or exogenous chemicals, or compounds that may or may not be deleterious to the major organs of the body. In principle the detoxification mechanisms function to attain this goal, however there are many cases of major organ toxicity upon exposure to drugs or metabolites of drugs. Although drugs and drug metabolites predominantly affect the liver and kidneys due to the circulatory and physiological processes, other organs can be affected. In the present invention, we address specific genes that may have polymorphic sites affecting metabolic rates to ultimately affect these major organ functions.

1. Excretion of Drugs and Drug Metabolites via the Bile

After parent drugs or xenobiotics are metabolized by redox and or conjugation reactions, the modified products can then be actively transported into the bile cannicula. The transport occurs in an energy dependent fashion requiring ATP. It has been shown that the transporters involved in the active transport from the basolateral (sinusoidal) to the apical (canalicular) surfaces of hepatocytes are members of the ATP binding cassette (ABC) family. The transmembrane electrical potential required to maintain the chemical and electrical potentials required for this active transport is provided by the Na+/K+ ATPases located on the basolateral membrane. Other ion transporters are the potassium channel, sodium-bicarbonate symporter, chloride-bicarbonate anion exchanger, and the chloride channel. In the cholangiocyte there are other ion transporters, for example chloride-bicarbonate anion exchanger, isoform 2, and other organic-solute transporters. Bile acids, phosphatidyl chorine, organic anions, organic cations, and cholesterol are actively transported. Approximately 5% of the transporters is multi-drug resistance protein 1 (MDR1) and the remaining are the phospholipid transporter multi-drug resistance protein 3 (MDR3), alicular multispecific organic-anion transporter (multi-drug resistance associated protein (MRP2 or cMOAT), canalicular bile-salt-export pump (BSEP or SPGP(sister of p-glycoprotein)), sodium-taurocholate cotransporter, organic anion-transporting polypeptide, glutathione transporter, and a chloride-bicarbonate anion exchanger are also involved in the transport.

These transporters have been identified to move specific molecules or compounds across biological membranes. For example, the MDR1 protein mediates the canicular excretion of bulky lipophilic cations, e.g. anticancer drugs, calcium channel blockers, cyclosporine A, and various other drugs. In contrast, the MDR3 protein transports phosphatidyl choline from the inner leaflet to the outer leaflet of the canicular membrane. Phosphatidyl choline then can be selectively extracted by intracanicular bile salts and secreted into bile as vesicles or mixed micelles. MRP2 is involved in the transport of amphipathic anionic substrates e.g. leukotriene C4, glutathione-S conjugates, glucuronides (bilirubin diglucuronide and estradiol-17b-glucuronide), sulfate conjugates, and is responsible for the generation of bile flow independent of bile salts within the bile cannicula. SPGP is the canicular bile salt export pump in the mammalian liver.

The hepatocyte has the ability to recruit the ATP-requiring transporters when faced with excessive metabolites. After synthesis, these transporters are stored in compartments that, in response to cAMP, can be actively moved through the cell to the membrane and fused to the cannicula. The active movement from the intracellular compartment to the membrane requires microtubules, cytoplasmic kinesin, cytoplasmic dynesin, and calcium. It has been shown that peptides activate phophosinositide 3 kinase, and increased turnover of phosphoinostides drives the formation of 3′phophoinositol, which can activate the transporter in the membrane and ultimately increases movement to the cannicular membrane. Signaling pathways via the activation of rab5 stimulate the active movement of the transporters to the internal compartment.

2. Excretion of Drugs and Drug Metabolites via the Kidney

Excretion of drugs or drug metabolites via the kidney and into the urine involves three processes: 1) glomerular filtration, 2) active tubular secretion, and 3) passive tubular reabsorption. The amount of drug or metabolites entering the tubular lumen is dependent on its fractional plasma protein binding and glomerular filtration rate. In the proximal renal tubule anions and cations are actively transported by carrier mediated tubular secretion and bases are transported by a separate system that secretes choline, histamine, and other endogenous bases. In the proximal and distal tubules there is passive reabsorption of these molecules. The concentration gradient for back-diffusion is created by sodium and other inorganic ions and water.

IV. Identification of Interpatient Variation in Response; Identification of Genes and Variances Relevant to Drug Action; Development of Diagnostic Tests; and Use of Variance Status to Determine Treatment

Development of therapeutics in man follows a course from compound discovery and analysis in a laboratory (preclinical development) to testing the candidate therapeutic intervention in human subjects (clinical development). The preclinical development of candidate therapeutic interventions for use in the treatment of human diseases, disorders, or conditions begins at the discovery stage whereby a candidate therapy is tested in vitro to achieve a desired biochemical alteration of a biochemical or physiological event. If successful, the candidate is generally tested in animals to determine toxicity, adsorption, distribution, metabolism and excretion in a living species. Occasionally, there are available animal models that mimic human diseases, disorders, and conditions in which testing the candidate therapeutic intervention can provide supportive data to warrant proceeding to test the compound in humans. It is widely recognized that preclinical data is imperfect in predicting response to a compound in man. Both safety and efficacy have to ultimately be demonstrated in humans. Therefore, given economic constraints, and considering the complexities of human clinical trials, any technical advance that increases the likelihood of successfully developing and registering a compound, or getting new indications for a compound, or marketing a compound successfully against competing compounds or treatment regimens, will find immediate use. Indeed, there has been much written about the potential of pharmacogenetics to change the practice of medicine. In this application we provide descriptions of the methods one skilled in the art would use to advance compounds through clinical trials using genetic stratification as a tool to circumvent some of the difficulties typically encountered in clinical development, such as poor efficacy or toxicity. We also provide specific genes, variation in which may account for interpatient variation in treatment response, and further we provide specific exemplary variances in those genes that may account for variation in treatment response.

The study of sequence variation in genes that mediate and modulate the action of drugs may provide advances at virtually all stages of drug development. For example, identification of amino acid variances in a drug target during preclinical development would allow development of non-allele selective agents. During early clinical development, knowledge of variation in a gene related to drug action could be used to design a clinical trial in which the variances are taken account of by, for example, including secondary endpoints that incorporate an analysis of response rates in genetic subgroups. In later stages of clinical development the goal might be to first establish retrospectively whether a particular problem, such as liver toxicity, can be understood in terms of genetic subgroups, and thereby controlled using a genetic test to screen patients. Thus genetic analysis of drug response can aid successful development of therapeutic products at any stage of clinical development. Even after a compound has achieved regulatory approval its commercialization can be aided by the methods of this invention, for example by allowing identification of genetically defined responder subgroups in new indications (for which approval in the entire disease population could not be achieved) or by providing the basis for a marketing campaign that highlights the superior efficacy and/or safety of a compound coupled with a genetic test to identify preferential responders. Thus the methods of this invention will provide medical, economic and marketing advantages for products, and over the longer term increase therapeutic alternatives for patients.

As indicated in the Summary above, certain aspects of the present invention typically involve the following process, which need not occur separately or in the order stated. Not all of these described processes must be present in a particular method, or need be performed by a single entity or organization or person. Additionally, if certain of the information is available from other sources, that information can be utilized in the present invention. The processes are as follows: a) variability between patients in the response to a particular treatment is observed; b) at least a portion of the variable response is correlated with the presence or absence of at least one variance in at least one gene; c) an analytical or diagnostic test is provided to determine the presence or absence of the at least one variance in individual patients; d) the presence or absence of the variance or variances is used to select a patient for a treatment or to select a treatment for a patient, or the variance information is used in other methods described herein.

A. Identification of Interpatient Variability in Response to a Treatment

Interpatient variability is the rule, not the exception, in clinical therapeutics. One of the best sources of information on interpatient variability is the nurses and physicians supervising the clinical trial who accumulate a body of first hand observations of physiological responses to the drug in different normal subjects or patients. Evidence of interpatient variation in response can also be measured statistically, and may be best assessed by descriptive statistical measures that examine variation in response (beneficial or adverse) across a large number of subjects, including in different patient subgroups (men vs. women; whites vs. blacks; Northern Europeans vs. Southern Europeans, etc.).

In accord with the other portions of this description, the present invention concerns DNA sequence variances that can affect one or more of:

i. The susceptibility of individuals to a disease;

ii. The course or natural history of a disease;

iii. The response of a patient with a disease to a medical intervention, such as, for example, a drug, a biologic substance, physical energy such as radiation therapy, or a specific dietary regimen. (The terms ‘drug’, ‘compound’ or ‘treatment’ as used herein may refer to any of the foregoing medical interventions.) The ability to predict either beneficial or detrimental responses is medically useful.

Thus variation in any of these three parameters may constitute the basis for initiating a pharmacogenetic study directed to the identification of the genetic sources of interpatient variation. The effect of a DNA sequence variance or variances on disease susceptibility or natural history (i and ii, above) are of particular interest as the variances can be used to define patient subsets which behave differently in response to medical interventions such as those described in (iii). The methods of this invention are also useful in a clinical development program where there is not yet evidence of interpatient variation (perhaps because the compound is just entering clinical trials) but such variation in response can be reliably anticipated. It is more economical to design pharmacogenetic studies from the beginning of a clinical development program than to start at a later stage when the costs of any delay are likely to be high given the resources typically committed to such a program.

In other words, a variance can be useful for customizing medical therapy at least for either of two reasons. First, the variance may be associated with a specific disease subset that behaves differently with respect to one or more therapeutic interventions (i and ii above); second, the variance may affect response to a specific therapeutic intervention (iii above). Consider for exemplary purposes pharmacological therapeutic interventions. In the first case, there may be no effect of a particular gene sequence variance on the observable pharmacological action of a drug, yet the disease subsets defined by the variance or variances differ in their response to the drug because, for example, the drug acts on a pathway that is more relevant to disease pathophysiology in one variance-defined patient subset than in another variance-defined patient subset. The second type of useful gene sequence variance affects the pharmacological action of a drug or other treatment. Effects on pharmacological responses fall generally into two categories; pharmacokinetic and pharmacodynamic effects. These effects have been defined as follows in Goodman and Gilman's Pharmacologic Basis of Therapeutics (ninth edition, McGraw Hill, New York, 1986): “Pharmacokinetics” deals with the absorption, distribution, biotransformations and excretion of drugs. The study of the biochemical and physiological effects of drugs and their mechanisms of action is termed “pharmacodynamics.”

Useful gene sequence variances for this invention can be described as variances which partition patients into two or more groups that respond differently to a therapy or that correlate with differences in disease susceptibility or progression, regardless of the reason for the difference, and regardless of whether the reason for the difference is known. The latter is true because it is possible, with genetic methods, to establish reliable associations even in the absence of a pathophysiological hypothesis linking a gene to a phenotype, such as a pharmacological response, disease susceptibility or disease prognosis.

B. Identification of Specific Genes and Correlation of Variances in Those Genes with Response to Treatment of Diseases or Conditions

It is useful to identify particular genes which do or are likely to mediate the efficacy or safety of a treatment method for a disease or condition, particularly in view of the large number of genes which have been identified and which continue to be identified in humans. As is further discussed in section C below, this correlation can proceed by different paths. One exemplary method utilizes prior information on the pharmacology or pharmacokinetics or pharmacodynamics of a treatment method, e.g., the action of a drug, which indicates that a particular gene is, or is likely to be, involved in the action of the treatment method, and further suggests that variances in the gene may contribute to variable response to the treatment method. For example if a compound is known to be glucuronidated then a glucuronyltransferase is likely involved. If the compound is a phenol, the likely glucuronyltransferase is UGT1 (either the UGT1*1 or UGT1*6 transcripts, both of which catalyze the conjugation of planar phenols with glucuronic acid). Similar inferences can be made for many other biotransformation reactions.

Alternatively, if such information is not known, variances in a gene can be correlated empirically with treatment response. In this method, variances in a gene which exist in a population can be identified. The presence of the different variances or haplotypes in individuals of a study group, which is preferably representative of a population or populations of known geographic, ethnic and/or racial background, is determined. This variance information is then correlated with treatment response of the various individuals as an indication that genetic variability in the gene is at least partially responsible for differential treatment response. It may be useful to independently analyze variances in the different geographic, ethnic and/or racial groups as the presence of different genetic variances in these groups (i.e. different genetic background) may influence the effect of a specific variance. That is, there may be a gene×gene interaction involving one unstudied gene, however the indicated demographic variables may act as a surrogate for the unstudied allele. Statistical measures known to those skilled in the art are preferably used to measure the fraction of interpatient variation attributable to any one variance, or to measure the response rates in different subgroups defined genetically or defined by some combination of genetic, demographic and clinical criteria.

Useful methods for identifying genes relevant to the pharmacological action of a drug or other treatment are known to those skilled in the art, and include review of the scientific literature combined with inferential or deductive reasoning that one skilled in the art of molecular pharmacology and molecular biology would be capable of; large scale analysis of gene expression in cells treated with the drug compared to control cells; large scale analysis of the protein expression pattern in treated vs. untreated cells, or the use of techniques for identification of interacting proteins or ligand-protein interactions, such as yeast two-hybrid systems.

C. Development of a Diagnostic Test to Determine Variance Status

In accordance with the description in the Summary above, the present invention generally concerns the identification of variances in genes which are indicative of the effectiveness of a treatment in a patient. The identification of specific variances, in effect, can be used as a diagnostic or prognostic test. Correlation of treatment efficacy and/or toxicity with particular genes and gene families or pathways is provided in Stanton et al., U.S. Provisional Application Ser. No. 60/093,484, filed Jul. 20, 1998, entitled GENE SEQUENCE VARIANCES WITH UTILITY IN DETERMINING THE TREATMENT OF DISEASE (concerns the safety and efficacy of compounds active on folate or pyrimidine metabolism or action) and Stanton, U.S. Provisional Application Ser. No. 60/121,047, filed Feb. 22, 1999, entitled GENE SEQUENCE VARIANCES WITH UTILITY IN DETERMINING THE TREATMENT OF DISEASE (concerning Alzheimer's disease and other dementias and cognitive disorders), which are hereby incorporated by reference in their entireties including drawings.

Genes identified in the examples below and in the Tables and Figures can be used in the methods of the present invention. A variety of genes which the inventors realize may account for interpatient variation in patient outcome response to candidate therapeutic interventions are listed in Tables 1, 3, and 4. Gene sequence variances in said genes are particularly useful for aspects of the present invention.

Methods for diagnostic tests are well known in the art. Generally in this invention, the diagnostic test involves determining whether an individual has a variance or variant form of a gene that is involved in the disease or condition or the action of the drug or other treatment or effects of such treatment. Such a variance or variant form of the gene is preferably one of several different variances or forms of the gene that have been identified within the population and are known to be present at a certain frequency. In an exemplary method, the diagnostic test involves determining the sequence of at least one variance in at least one gene after amplifying a segment of said gene using a DNA amplification method such as the polymerase chain reaction (PCR). In this method DNA for analysis is obtained by amplifying a segment of DNA or RNA (generally after converting the RNA to cDNA) spanning one or more variances in the gene sequence. Preferably, the amplified segment is <500 bases in length, in an alternative embodiment the amplified segment is <100 bases in length, most preferably <45 bases in length.

In some cases it will be desirable to determine a haplotype instead of a genotype. In such a case the diagnostic test is performed by amplifying a segment of DNA or RNA (cDNA) spanning more than one variance in the gene sequence and preferably maintaining the phase of the variances on each allele. The term “phase” refers to the relationship of variances on a single chromosomal copy of the gene, such as the copy transmitted from the mother (maternal copy or maternal allele) or the father (paternal copy or paternal allele). The haplotyping test may take part in two phases, where first genotyping tests at two or more variant sites reveal which sites are heterozygous in each patient or normal subject. Subsequently the phase of the two or more variant sites can be determined. In performing a haplotyping test preferably the amplified segment is >500 bases in length, more preferably it is >1,000 bases in length, and most preferably it is >2,500 bases in length. One way of preserving phase is to amplify one strand in the PCR reaction. This can be done using one or a pair of oligonucleotide primers that terminate (i.e. have a 3′ end that stops) opposite the variant site, such that one primer is perfectly complementary to one variant form and the other primer is perfectly complementary to the other variant form. Other than the difference in the 3′ most nucleotide the two primers are identical (forming an allelic primer pair). Only one of the allelic primers is used in any PCR reaction, depending on which strand is being amplified. The primer for the opposite strand may also be an allelic primer, or it may prime from a non-polymorphic region of the template. This method exploits the requirement of most polymerases for perfect complementarity at the 3′ terminus of the primer in a primer-template complex. See, for example: Lo Y M, Patel P, Newton C R, Markham A F, Fleming K A and J S Wainscoat. (1991) Direct haplotype determination by double ARMS: specificity, sensitivity and genetic applications. Nucleic Acids Res July 11;19(13):3561-7.

It is apparent that such diagnostic tests are performed after initial identification of variances within the gene, which allows selection of appropriate allele specific primers.

Diagnostic genetic tests useful for practicing this invention belong to two types: genotyping tests and haplotyping tests. A genotyping test simply provides the status of a variance or variances in a subject or patient. For example suppose nucleotide 150 of hypothetical gene X on an autosomal chromosome is an adenine (A) or a guanine (G) base. The possible genotypes in any individual are AA, AG or GG at nucleotide 150 of gene X.

In a haplotyping test there is at least one additional variance in gene X, say at nucleotide 810, which varies in the population as cytosine (C) or thymine (T). Thus a particular copy of gene X may have any of the following combinations of nucleotides at positions 150 and 810: 150A-810C, 150A-810T, 150G-810C or 150G-810T. Each of the four possibilities is a unique haplotype. If the two nucleotides interact in either RNA or protein, then knowing the haplotype can be important. The point of a haplotyping test is to determine the haplotypes present in a DNA or cDNA sample (e.g. from a patient). In the example provided there are only four possible haplotypes, but, depending on the number of variances in the gene and their distribution in human populations there may be three, four, five, six or more haplotypes at a given gene. The most useful haplotypes for this invention are those which occur commonly in the population being treated for a disease or condition. Preferably such haplotypes occur in at least 5% of the population, more preferably in at least 10%, still more preferably in at least 20% of the population and most preferably in at least 30% or more of the population. Conversely, when the goal of a pharmacogenetic program is to identify a relatively rare population that has an adverse reaction to a treatment, the most useful haplotypes may be rare haplotypes, which may occur in less than 5%, less than 2%, or even in less than 1% of the population. One skilled in the art will recognize that the frequency of the adverse reaction provides a useful guide to the likely frequency of salient causative haplotypes.

Based on the identification of variances or variant forms of a gene, a diagnostic test utilizing methods known in the art can be used to determine whether a particular form of the gene, containing specific variances or haplotypes, or combinations of variances and haplotypes, is present in at least one copy, one copy, or more than one copy in an individual. Such tests are commonly performed using DNA or RNA collected from blood, cells, tissue scrapings or other cellular materials, and can be performed by a variety of methods including, but not limited to, PCR based methods, hybridization with allele-specific probes, enzymatic mutation detection, chemical cleavage of mismatches, mass spectrometry or DNA sequencing, including minisequencing. Methods for haplotyping are described above. In particular embodiments, hybridization with allele specific probes can be conducted in two formats: (1) allele specific oligonucleotides bound to a solid phase (glass, silicon, nylon membranes) and the labeled sample in solution, as in many DNA chip applications, or (2) bound sample (often cloned DNA or PCR amplified DNA) and labeled oligonucleotides in solution (either allele specific or short—e.g. 7mers or 8mers—so as to allow sequencing by hybridization). Preferred methods for diagnostic testing of variances are described in four patent applications Stanton et al, entitled A METHOD FOR ANALYZING POLYNUCLEOTIDES, Ser. Nos. 09/394,467; 09/394,457; 09/394,774; and 09/394,387; all filed Sep. 10, 1999. The application of such diagnostic tests is possible after identification of variances that occur in the population. Diagnostic tests may involve a panel of variances from one or more genes, often on a solid support, which enables the simultaneous determination of more than one variance in one or more genes.

D. Use of Variance Status to Determine Treatment

The present disclosure describes exemplary gene sequence variances in genes identified in a gene table herein (e.g., Tables 3 and 4), and variant forms of these gene that may be determined using diagnostic tests. As indicated in the Summary, such a variance-based diagnostic test can be used to determine whether or not to administer a specific drug or other treatment to a patient for treatment of a disease or condition. Preferably such diagnostic tests are incorporated in texts such as are described in Clinical Diagnosis and Management by Laboratory Methods (19th Ed) by John B. Henry (Editor) W B Saunders Company, 1996; Clinical Laboratory Medicine : Clinical Application of Laboratory Data, (6th edition) by R. Ravel, Mosby-Year Book, 1995, or other medical textbooks including, without limitation, textbooks of medicine, laboratory medicine, therapeutics, pharmacy, pharmacology, nutrition, allopathic, homeopathic, and osteopathic medicine; preferably such a test is developed as a ‘home brew’ method by a certified diagnostic laboratory; most preferably such a diagnostic test is approved by regulatory authorities, e.g., by the U.S. Food and Drug Administration, and is incorporated in the label or insert for a therapeutic compound, as well as in the Physicians Desk Reference.

In such cases, the procedure for using the drug is restricted or limited on the basis of a diagnostic test for determining the presence of a variance or variant form of a gene. Alternatively the use of a genetic test may be advised as best medical practice, but not absolutely required, or it may be required in a subset of patients, e.g. those using one or more other drugs, or those with impaired liver or kidney function. The procedure that is dictated or recommended based on genotype may include the route of administration of the drug, the dosage form, dosage, schedule of administration or use with other drugs; any or all of these may require selecting or determination consistent with the results of the diagnostic test or a plurality of such tests. Preferably the use of such diagnostic tests to determine the procedure for administration of a drug is incorporated in a text such as those listed above, or medical textbooks, for example, textbooks of medicine, laboratory medicine, therapeutics, pharmacy, pharmacology, nutrition, allopathic, homeopathic, and osteopathic medicine. As previously stated, preferably such a diagnostic test or tests are required by regulatory authorities and are incorporated in the label or insert as well as the Physicians Desk Reference.

Variances and variant forms of genes useful in conjunction with treatment methods may be associated with the origin or the pathogenesis of a disease or condition. In many useful cases, the variant form of the gene is associated with a specific characteristic of the disease or condition that is the target of a treatment, most preferably response to specific drugs or other treatments. Examples of diseases or conditions ameliorable by the methods of this invention are identified in the Examples and tables below; in general treatment of disease with current methods, particularly drug treatment, always involves some unknown element (involving efficacy or toxicity or both) that can be reduced by appropriate diagnostic methods.

Alternatively, the gene is involved in drug action, and the variant forms of the gene are associated with variability in the action of the drug. For example, in some cases, one variant form of the gene is associated with the action of the drug such that the drug will be effective in an individual who inherits one or two copies of that form of the gene. Alternatively, a variant form of the gene is associated with the action of the drug such that the drug will be toxic or otherwise contra-indicated in an individual who inherits one or two copies of that form of the gene.

In accord with this invention, diagnostic tests for variances and variant forms of genes as described above can be used in clinical trials to demonstrate the safety and efficacy of a drug in a specific population. As a result, in the case of drugs which show variability in patient response correlated with the presence or absence of a variance or variances, it is preferable that such drug is approved for sale or use by regulatory agencies with the recommendation or requirement that a diagnostic test be performed for a specific variance or variant form of a gene which identifies specific populations in which the drug will be safe and/or effective. For example, the drug may be approved for sale or use by regulatory agencies with the specification that a diagnostic test be performed for a specific variance or variant form of a gene which identifies specific populations in which the drug will be toxic. Thus, approved use of the drug, or the procedure for use of the drug, can be limited by a diagnostic test for such variances or variant forms of a gene; or such a diagnostic test may be considered good medical practice, but not absolutely required for use of the drug.

As indicated, diagnostic tests for variances as described in this invention may be used in clinical trials to establish the safety and efficacy of a drug. Methods for such clinical trials are described below and/or are known in the art and are described in standard textbooks. For example, diagnostic tests for a specific variance or variant form of a gene may be incorporated in the clinical trial protocol as inclusion or exclusion criteria for enrollment in the trial, to allocate certain patients to treatment or control groups within the clinical trial or to assign patients to different treatment cohorts. Alternatively, diagnostic tests for specific variances may be performed on all patients within a clinical trial, and statistical analysis performed comparing and contrasting the efficacy or safety of a drug between individuals with different variances or variant forms of the gene or genes. Preferred embodiments involving clinical trials include the genetic stratification strategies, phases, statistical analyses, sizes, and other parameters as described herein.

Similarly, diagnostic tests for variances can be performed on groups of patients known to have efficacious responses to the drug to identify differences in the frequency of variances between responders and non-responders. Likewise, in other cases, diagnostic tests for variance are performed on groups of patients known to have toxic responses to the drug to identify differences in the frequency of the variance between those having adverse events and those not having adverse events. Such outlier analyses may be particularly useful if a limited number of patient samples are available for analysis. It is apparent that such clinical trials can be or are performed after identifying specific variances or variant forms of the gene in the population. In defining outliers it is useful to examine the distribution of responses in the placebo group; outliers should preferably have responses that exceed in magnitude the extreme responses in the placebo group.

The identification and confirmation of genetic variances is described in certain patents and patent applications. The description therein is useful in the identification of variances in the present invention. For example, a strategy for the development of anticancer agents having a high therapeutic index is described in Housman, International Application PCT/US/94 08473 and Housman, INHIBITORS OF ALTERNATIVE ALLELES OF GENES ENCODING PROTEINS VITAL FOR CELL VIABILITY OR CELL GROWTH AS A BASIS FOR CANCER THERAPEUTIC AGENTS, U.S. Pat. No. 5,702,890, issued Dec. 30, 1997, which are hereby incorporated by reference in their entireties. Also, a number of gene targets and associated variances are identified in Housman et al., U.S. patent application Ser. No. 09/045,053, entitled TARGET ALLELES FOR ALLELE-SPECIFIC DRUGS, filed Mar. 19, 1998, which is hereby incorporated by reference in its entirety, including drawings.

The described approach and techniques are applicable to a variety of other diseases, conditions, and/or treatments and to genes associated with the etiology and pathogenesis of such other diseases and conditions and the efficacy and safety of such other treatments.

Useful variances for this invention can be described generally as variances which partition patients into two or more groups that respond differently to a therapy (a therapeutic intervention), regardless of the reason for the difference, and regardless of whether the reason for the difference is known.

III. From Variance List to Clinical Trial: Identifying Genes and Gene Variances that Account for Variable Responses to Treatment

There are a variety of useful methods for identifying a subset of genes from a large set of candidate genes that should be prioritized for further investigation with respect to their influence on inter-individual variation in disease predisposition or response to a particular drug. These methods include for example, (1) searching the biomedical literature to identify genes relevant to a disease or the action of a drug, (2) screening the genes identified in step 1 for variances. A large set of exemplary variances are provided in Tables 3 and 4. Other methods include (3) using computational tools to predict the functional effects of variances in specific genes, (4) using in vitro or in vivo experiments to identify genes which may participate in the response to a drug or treatment, and to determine the variances which affect gene, RNA or protein function, and may therefore be important genetic variables affecting disease manifestations or drug response, and (5) retrospective or prospective clinical trials. Computational tools are described in U.S. patent application, Stanton et al., Ser. No. 09/300,747, filed Apr. 26, 1999, entitled GENE SEQUENCE VARIANCES WITH UTILITY IN DETERMINING THE TREATMENT OF DISEASE, and in Stanton et al., Ser. No. 09/419,705, filed Oct. 14, 1999, entitled VARIANCE SCANNING METHOD FOR IDENTIFYING GENE SEQUENCE VARIANCES, which are hereby incorporated by reference in their entireties, including drawings. Other methods are considered below in some detail.

(1) To begin, one preferably identifies, for a given treatment, a set of candidate genes that are likely to affect disease phenotype or drug response. This can be accomplished most efficiently by first assembling the relevant medical, pharmacological and biological data from available sources (e.g., public databases and publications). One skilled in the art can review the literature (textbooks, monographs, journal articles) and online sources (databases) to identify genes most relevant to the action of a specific drug or other treatment, particularly with respect to its utility for treating a specific disease, as this beneficially allows the set of genes to be analyzed ultimately in clinical trials to be reduced from an initial large set. Specific strategies for conducting such searches are described below. In some instances the literature may provide adequate information to select genes to be studied in a clinical trial, but in other cases additional experimental investigations of the sort described below will be preferable to maximize the likelihood that the salient genes and variances are moved forward into clinical studies. Specific genes relevant to understanding interpatient variation in patient outcome response to candidate therapeutic interventions are listed in Table 1. In Table 2 preferred sets of genes for analysis of variable therapeutic response in specific diseases are highlighted. These genes are exemplary; they do not constitute a complete set of genes that may account for variation in clinical response. Experimental data are also useful in establishing a list of candidate genes, as described below.

(2) Having assembled a list of candidate genes generally the second step is to screen for variances in each candidate gene. Experimental and computational methods for variance detection are described in this invention, and tables of exemplary variances are provided (Tables 3, and 4) as well as methods for identifying additional variances and a written description of such possible additional variances in the cDNAs of genes that may affect drug action (see Stanton & Adams, application Ser. No. 09/300,747, filed Apr. 26, 1999, entitled GENE SEQUENCE VARIANCES WITH UTILITY IN DETERMINING THE TREATMENT OF DISEASE, incorporated in its entirety.

(3) Having identified variances in candidate genes the next step is to assess their likely contribution to clinical variation in patient response to therapy, preferably by using informatics-based approaches such as DNA and protein sequence analysis and protein modeling. The literature and informatics-based approaches provide the basis for prioritization of candidate genes, however it may in some cases be desirable to further narrow the list of candidate genes, or to measure experimentally the phenotype associated with specific variances or sets of variances (e.g. haplotypes).

(4) Thus, as a third step in candidate gene analysis, one skilled in the art may elect to perform in vitro or in vivo experiments to assess the functional importance of gene variances, using either biochemical or genetic tests. (Certain kinds of experiments—for example gene expression profiling and proteome analysis—may not only allow refinement of a candidate gene list but may also lead to identification of additional candidate genes.) Combination of two or all of the three above methods will provide sufficient information to narrow and prioritize the set of candidate genes and variances to a number that can be studied in a clinical trial with adequate statistical power.

(5) The fourth step is to design retrospective or prospective human clinical trials to test whether the identified allelic variance, variances, or haplotypes or combination thereof influence the efficacy or toxicity profiles for a given drug or other therapeutic intervention. It should be recognized that this fourth step is the crucial step in producing the type of data that would justify introducing a diagnostic test for at least one variance into clinical use. Thus while each of the above four steps are useful in particular instances of the invention, this final step is indispensable. Further guidance and examples of how to perform these five steps are provided below.

(6) A fifth (optional) step entails methods for using a genotyping test in the promotion and marketing of a treatment method. It is widely appreciated that there is a tendency in the pharmaceutical industry to develop many compounds for well established therapeutic targets. Examples include beta adrenergic blockers, hydroxymethylglutaryl (HMG) CoA reductase inhibitors (statins), dopamine D2 receptor antagonists and serotonin transporter inhibitors. Frequently the pharmacology of these compounds is quite similar in terms of efficacy and side effects. Therefore the marketing of one compound vs. other members of the class is a challenging problem for drug companies, and is reflected in the lesser success that late products typically achieve compared to the first and second approved products. It occurred to the inventors that genetic stratification can provide the basis for identifying a patient population with a superior response rate or improved safety to one member of a class of drugs, and that this information can be the basis for commercialization of that compound. Such a commercialization campaign can be directed at caregivers, particularly physicians, or at patients and their families, or both.

1. Identification of Candidate Genes Relevant to the Action of a Drug

Practice of this invention will often begin with identification of a specific pharmaceutical product, for example a drug, that would benefit from improved efficacy or reduced toxicity or both, and the recognition that pharmacogenetic investigations as described herein provide a basis for achieving such improved characteristics. The question then becomes which genes and variances, such as those provided in this application in Tables 1, 3, and 4, would be most relevant to interpatient variation in response to the drug. As discussed above, the set of relevant genes includes both genes involved in the disease process and genes involved in the interaction of the patient and the treatment—for example genes involved in pharmacokinetic and pharmacodynamic action of a drug. The biological and biomedical literature and online databases provide useful guidance in selecting such genes. Specific guidance in the use of these resources is provided below.

Review the Literature and Online Sources

One way to find genes that affect response to a drug in a particular disease setting is to review the published literature and available online databases regarding the pathophysiology of the disease and the pharmacology of the drug. Literature or online sources can provide specific genes involved in the disease process or drug response, or describe biochemical pathways involving multiple genes, each of which may affect the disease process or drug response.

Alternatively, biochemical or pathological changes characteristic of the disease may be described; such information can be used by one skilled in the art to infer a set of genes that can account for the biochemical or pathologic changes. For example, to understand variation in response to a drug that modulates serotonin levels in a central nervous system (CNS) disorder associated with altered levels of serotonin one would preferably study, at a minimum, variances in genes responsible for serotonin biosynthesis, release from the cell, receptor binding, presynaptic reuptake, and degradation or metabolism. Genes responsible for each of these functions should be examined for variation that may account for interpatient differences in drug response or disease manifestations. As recognized by those skilled in the art, a comprehensive list of such genes can be obtained from textbooks, monographs and the literature.

There are several types of scientific information, described in some detail below, that are valuable for identifying a set of candidate genes to be investigated with respect to a specific disease and therapeutic intervention. First there is the medical literature, which provides basic information on disease pathophysiology and therapeutic interventions. A subset of this literature is devoted to specific description of pathologic conditions. Second there is the pharmacology literature, which will provide additional information on the mechanism of action of a drug (pharmacodynamics) as well as its principal routes of metabolic transformation (pharmacokinetics) and the responsible proteins. Third there is the biomedical literature (principally genetics, physiology, biochemistry and molecular biology), which provides more detailed information on metabolic pathways, protein structure and function and gene structure. Fourth, there are a variety of online databases that provide additional information on metabolic pathways, gene families, protein function and other subjects relevant to selecting a set of genes that are likely to affect the response to a treatment.

Medical Literature

A good starting place for information on molecular pathophysiology of a specific disease is a general medical textbook such as Harrison's Principles of Internal Medicine, 14th edition, (2 Vol Set) by A. S. Fauci, E. Braunwald, K. J. Isselbacher, et al. (editors), McGraw Hill, 1997, or Cecil Textbook of Medicine (20th Ed) by R. L. Cecil, F. Plum and J. C. Bennett (Editors) W B Saunders Co., 1996. For pediatric diseases texts such as Nelson Textbook of Pediatrics (15th edition) by R. E. Behrman, R. M. Kliegman, A. M. Arvin and W. E. Nelson (Editors), W B Saunders Co., 1995 or Oski's Principles and Practice of Pediatrics (3^{rd} Edition) by J. A. Mamillan & F. A. Oski Lippincott-Raven, 1999 are useful introductions. For obstetrical and gynecological disorders texts such as Williams Obstetrics (20th Ed) by F. G. Cunningham, N. F. Gant, P. C. McDonald et al. (Editors), Appleton & Lange, 1997 provide general information on disease pathophysiology. For psychiatric disorders texts such as the Comprehensive Textbook of Psychiatry, VI (2 Vols) by H. I. Kaplan and B. J. Sadock (Editors), Lippincott, Williams & Wilkins, 1995, or The American Psychiatric Press Textbook of Psychiatry (3^{rd} edition) by R. E. Hales, S. C. Yudofsky and J. A. Talbott (Editors) Amer Psychiatric Press, 1999 provide an overview of disease nosology, pathophysiological mechanisms and treatment regimens.

In addition to these general texts, there are a variety of more specialized medical texts that provide greater detail about specific disorders which can be utilized in developing a list of candidate genes and variances relevant to interpatient variation in response to a treatment. For example, within the field of medicine there are standard textbooks for each of the subspecialties. Some specific examples include:

Heart Disease: A Textbook of Cardiovascular Medicine (2 Volume set) by E. Braunwald (Editor), W B Saunders Co., 1996.

Hurst's the Heart, Arteries and Veins (9th Ed) (2 Vol Set) by R. W. Alexander, R. C. Schlant, V. Fuster, W. Alexander and E. H. Sonnenblick (Editors) McGraw Hill, 1998.

Principles of Neurology (6th edition) by R. D. Adams, M. Victor (editors), and A. H. Ropper (Contributor), McGraw Hill, 1996.

Sleisenger & Fordtran's Gastrointestinal and Liver Disease: Pathophysiology Diagnosis, Management (6th edition) by M. Feldman, B. F. Scharschmidt and M. Sleisenger (Editors), W B Saunders Co., 1997.

Textbook of Rheumatology (5th edition) by W. N. Kelley, S. Ruddy, E. D. Harris Jr. and C. B. Sledge (Editors) (2 volume set) W B Saunders Co., 1997.

Williams Textbook of Endocrinology (9th edition) by J. D. Wilson, D. W. Foster, H. M. Kronenberg and Larsen (Editors), W B Saunders Co., 1998.

Wintrobe's Clinical Hematology (10th Ed) by G. R. Lee, J. Foerster (Editor) and J. Lukens (Editors) (2 Volumes) Lippincott, Williams & Wilkins, 1998.

Cancer: Principles & Practice of Oncology (5th edition) by V. T. Devita, S. A. Rosenberg and S. Hellman (editors), Lippincott-Raven Publishers, 1997.

Principles of Pulmonary Medicine (3rd edition) by S. E. Weinberger & J Fletcher (Editors), W B Saunders Co., 1998.

Diagnosis and Management of Renal Disease and Hypertension (2nd edition) by A. K. Mandal & J. C. Jennette (Editors), Carolina Academic Press, 1994.Massry & Glassock's Textbook of Nephrology (3rd edition) by S. G. Massry & R. J. Glassock (editors) Williams & Wilkins, 1995.

The Management of Pain by J. J. Bonica, Lea and Febiger, 1992

Ophthalmology by M. Yanoff & J. S. Duker, Mosby Year Book, 1998

Clinical Ophthalmology: A Systemic Approach by J. J. Kanski, Butterworth-Heineman, 1994.Essential Otolaryngoloy by J. K. Lee Appleton and Lange 1998.

In addition to these subspecialty texts there are many textbooks and monographs that concern more restricted disease areas, or specific diseases. Such books provide more extensive coverage of pathophysiologic mechanisms and therapeutic options. The number of such books is too great to provide examples for all but a few diseases, however one skilled in the art will be able to readily identify relevant texts. One simple way to search for relevant titles is to use the search engine of an online bookseller such as http://www.amazon.com or http://www.barnesandnoble.com using the disease or drug (or the group of diseases or drugs to which they belong) as search terms. For example a search for asthma would turn up titles such as Asthma: Basic Mechanisms and Clinical Management (3rd edition) by P. J. Barnes, I. W. Rodger and N. C. Thomson (Editors), Academic Press, 1998 and Airways and Vascular Remodelling in Asthma and Cardiovascular Disease: Implications for Therapeutic Intervention, by C. Page & J. Black (Editors), Academic Press, 1994.

Pathology Literature

In addition to medical texts there are texts that specifically address disease etiology and pathologic changes associated with disease. A good general pathology text is Robbins Pathologic Basis of Disease (6th edition) by R. S. Cotran, V. Kumar, T. Collins and S. L. Robbins, W B Saunders Co., 1998. Specialized pathology texts exist for each organ system and for specific diseases, similar to medical texts. These texts are useful sources of information for one skilled in the art for developing lists of genes that may account for some of the known pathologic changes in disease tissue. Exemplary texts are as follows:

Bone Marrow Pathology 2^{nd} edition, by B. J. Bain, I. Lampert. & D. Clark, Blackwell Science, 1996

Atlas of Renal Pathology by F. G. Silva, W. B. Saunders, 1999.

Fundamentals of Toxicologic Pathology by W. M. Haschek and C. G. Rousseaux, Academic Press, 1997.

Gastrointestinal Pathology by P. Chandrasoma, Appleton and Lange, 1998.

Ophthalmic Pathology with Clinical Correlations by J. Sassani, Lippincott-Raven, 1997.

Pathology of Bone and Joint Disorders by F. McCarthy, F. J. Frassica and A. Ross, W. B. Saunders, 1998.

Pulmonary Pathology by M. A. Grippi, Lippicott-Raven, 1995.

Neuropathology by D. Ellison, L. Chimelli, B. Harding, S. Love & J. Lowe, Mosby Year Book, 1997.

Greenfield's Neuropatholgy 6^{th} edition by J. G. Greenfield, P. L. Lantos & D. I. Graham, Edward Arnold, 1997.

Pharmacology, Pharmacogenetics and Pharmacy Literature

There are also both general and specialized texts and monographs on pharmacology that provide data on pharmacokinetics and pharmacodynamics of drugs. The discussion of pharmacodynamics (mechanism of action of the drug) in such texts is often supported by a review of the biochemical pathway or pathways that are affected by the drug. Also, proteins related to the target protein are often listed; it is important to account for variation in such proteins as the related proteins may be involved in drug pharmacology. For example, there are 14 known serotonin receptors. Various pharmacological serotonin agonists or antagonists have different affinities for these different receptors. Variation in a specific receptor may affect the pharmacology not only of drugs targeted to that receptor, but also drugs that are principally agonists or antagonists of different receptors. Such compounds may produce different effects on two allelic forms of a non-targeted receptor; for example on variant form may bind the compound with higher affinity than the other, or a compound that is principally an antagonist for one allele may be a partial agonist for another allele. Thus genes encoding proteins structurally related to the target protein should be screened for variance in order to successfully realize the methods of the present invention. A good general pharmacology text is Goodman & Gilman's the Pharmacological Basis of Therapeutics (9th Ed) by J. G. Hardman, L. E. Limbird, P. B. Molinoff, R. W. Ruddon and A. G. Gilman (Editors) McGraw Hill, 1996. There are also texts that focus on the pharmacology of drugs for specific disease areas, or specific classes of drugs (e.g. natural products) or adverse drug interactions, among other subjects. Specific examples include:

The American Psychiatric Press Textbook of Psychopharmacology (2nd edition) by A. F. Schatzberg & C. B. Nemeroff (Editors), American Psychiatric Press, 1998.

Essential Psychopharmacology: Neuroscientific Basis and Practical Applications by N. Muntner and S. M. Stahl, Cambridge Univ Press, 1996.

There are also texts on pharmacogenetics which are particularly useful for identifying genes which may contribute to variable pharmacokinetic response. In addition there are texts on some of the major xenobiotic metabolizing proteins, such as the cytochrome P450 genes.

Pharmacogenetics of Drug Metabolism (International Encyclopedia of Pharmacology and Therapeutics) by Werner Kalow (Editor) Pergamon Press, 1992.

Genetic Factors in Drug Therapy: Clinical and Molecular Pharmacogenetics by D. A Price Evans, Cambridge Univ Press, 1993.

Pharmacogenetics (Oxford Monographs on Medical Genetics, 32) by W. W. Weber, Oxford Univ Press, 1997.

Cytochrome P450: Structure, Mechanism, and Biochemistry by P. R. Ortiz de Montellano (Editor), Plenum Publishing Corp, 1995.

Appleton & Lange's Review of Pharmacy, 6^{th} edition, (Appleton & Lange's Review Series) by G. D. Hall & B. S. Reiss, Appleton & Lange, 1997.

Genetics, Biochemistry and Molecular Biology Literature

In addition to the medical, pathology, and pharmacology texts listed above there are several information sources that one skilled in the art will turn to for information on the genetic, physiologic, biochemical, and molecular biological aspects of the disease, disorder or condition or the effect of the therapeutic intervention on specific physiologic processes. The biomedical literature may include information on nonhuman organisms that is relevant to understanding the likely disease or pharmacological pathways in man.

Also provided below are illustrative texts which will aid in the identification of a pathway or pathways, and a gene or genes that may be relevant to interindividual variation in response to a therapy. Textbooks of biochemistry, genetics and physiology are often useful sources for such pathway information. In order to ascertain the appropriate methods to analyze the effects of an allelic variance, variances, or haplotypes in vitro, one skilled in the art will review existing information on molecular biology, cell biology, genetics, biochemistry; and physiology. Such texts are useful sources for general and specific information on the genetic and biochemical processes involved in disease and in drug action, as well as experimental procedures that may be useful in performing in vitro research on an allelic variance, variances, or haplotype.

Texts on gene structure and function and RNA biochemistry will be useful in evaluating the consequences of variances that do not change the coding sequence (silent variances). Such variances may alter the interaction of RNA with proteins or other regulatory molecules affecting RNA processing, polyadenylation, or export.

Molecular and Cellular Biology

Molecular Cell Biology by H. Lodish, D. Baltimore, A. Berk, L. Zipurksy & J. Damell, W H Freeman & Co., 1995.

Essentials of Molecular Biology, D. Freifelder and MalacinskiJones and Bartlett, 1993.

Genes and Genomes: A Changing Perspective, M. Singer and P. Berg, 1991. University Science Books

Gene Structure and Expression, J. D. Hawkins, 1996. Cambridge University Press

Molecular Biology of the Cell, 2nd edition, B. Alberts et al., Garland Publishing, 1994.

Molecular Genetics

The Metabolic and Molecular Bases of Inherited Disease by C. R. Scriver, A. L. Beaudet, W. S. Sly (Editors), 7th edition, McGraw Hill, 1995

Genetics and Molecular Biology, R. Schleif, 1994. 2nd edition, Johns Hopkins University Press

Genetics, P. J. Russell, 1996. 4th edition, Harper Collins

An Introduction to Genetic Analysis, Griffiths et al. 1993. 5th edition, W. H. Freeman and Company

Understanding Genetics: A molecular approach, Rothwell, 1993. Wiley-Liss

General Biochemistry

Biochemistry, L. Stryer, 1995. W. H. Freeman and Company

Biochemistry, D. Voet and J. G. Voet, 1995. John Wiley and Sons

Principles of Biochemistry, A. L. Lehninger, D. L. Nelson, and M. M. Cox, 1993. Worth Publishers

Biochemistry, G. Zubay, 1998. Wm. C. Brown Communications

Biochemistry, C. K. Mathews and K. E. van Holde, 1990. Benjamin/Cummings

Transcription

Eukaryotic Transcriptiuon Factors, D. S. Latchman, 1995. Academic Press

Eukaryotic Gene Transcription, S. Goodboum (ed.), 1996. Oxford University Press.

Transcription Factors and DNA Replication, D. S. Pederson and N. H. Heintz, 1994. CRC Press/R. G. Landes Company

Transcriptional Regulation, S. L. McKnight and K. Yamamoto (eds.), 1992. 2 volumes, Cold Spring Harbor Laboratory Press

RNA

Control of Messenger RNA Stability, J. Belasco and G. Brawerman (eds.), 1993. Academic Press

RNA-Protein Interactions, Nagai and Mattaj (eds.), 1994. Oxford University Press

mRNA Metabolism and Post-transcriptional Gene Regulation, Harford and Morris (eds.), 1997. Wiley-Liss

Translation

Translational Control, J. W. B. Hershey, M. B. Mathews, and N. Sonenberg (eds.), 1995. Cold Spring Harbor Laboratory Press

General Physiology

Textbook of Medical Physiology 9^{th} Edtion by A. C. Guyton and J. E. Hall W. B. Saunders, 1997

Review of Medical Physiology, 18^{th} Edition by W. F. Ganong, Appleton and Lange, 1997

Online Databases

Those skilled in the art are familiar with how to search the biomedical literature, such as, e.g., libraries, online PubMed, abstract listings, and online mutation databases. One particularly useful resource is maintained at the web site of the National Center for Biotechnology Information (ncbi): http:/www.ncbi.nlm.nih.gov/. From the ncbi site one can access Online Mendelian Inheritance in Man (OMIM),. OMIM can be found at: http://www3.ncbi.nlm.nih.gov/Omim/searchomim.html. OMIM is a medically oriented database of genetic information with entries for thousands of genes. The OMIM record number is provided for many of the genes in Tables 1, 3, and 4 (see column 3), and constitutes an excellent entry point for identification of references that point to the broader literature. Another useful site at NCBI is the Entrez browser, located at http://www3.ncbi.nlm.nih.gov/Entrez/. One can search genomes, polynucleotides, proteins, 3D structures, taxonomy or the biomedical literature (PubMed) via the Entrez site. More generally links to a number of useful sites with biomedical or genetic data are maintained at sites such as Med Web at the Emory University Health Sciences Center Library: http://WWW.MedWeb.Emory.Edu/MedWeb/: Riken, a Japanese web site at: http:/www.rtc.riken.go.jp/othersite.html with links to DNA sequence, structural, molecular biology, bioinformatics, and other databases; at the Oak Ridge National Laboratory web site: http://www.ornl.gov/hgmis/links.html; or at the Yahoo website of Diseases and Conditions: http://dir.yahoo.com/health/diseases and conditions/index.html. Each of the indicated web sites has additional useful links to other sites.

Another type of database with utility in selecting the genes on a biochemical pathway that may affect the response to a drug are databases that provide information on biochemical pathways. Examples of such databases include the Kyoto Encyclopedia of Genes and Genomes (KEGG), which can be found at: http://www.genome.ad.jp/kegg/kegg.html. This site has pictures of many biochemical pathways, as well as links to other metabolic databases such as the well known Boehringer Mannheim biochemical pathways charts: http://www.expasy.ch/cgi-bin/search-biochem-index. The metabolic charts at the latter site are comprehensive, and excellent starting points for working out the salient enzymes on any given pathway.

Each of the web sites mentioned above has links to other useful web sites, which in turn can lead to additional sites with useful information. Research Libraries

Those skilled in the art will often require information found only at large libraries. The National Library of Medicine (http://www.nlm.nih.gov/) is the largest medical library in the world and its catalogs can be searched online. Other libraries, such as university or medical school libraries are also useful to conduct searches. Biomedical books such as those referred to above can often be obtained from online bookstores as described above.

Biomedical Literature

To obtain up to date information on drugs and their mechanism of action and biotransformation; disease pathophysiology; biochemical pathways relevant to drug action and disease pathophysiology; and genes that encode proteins relevant to drug action and disease one skilled in the art will consult the biomedical literature. A widely used, publicly accessible web site for searching published journal articles is PubMed (http://www.ncbi.nlm.nih.gov/PubMed/). At this site, one can search for the most recent articles (within the last 1-2 months) or older literature (back to 1966). Many Journals also have their own sites on the world wide web and can be searched online. For example see the IDEAL web site at: http://www.apnet.com/www/ap/aboutid.html. This site is an online library, featuring full text journals from Academic Press and selected journals from W. B. Saunders and Churchill Livingstone. The site provides access (for a fee) to nearly 2000 scientific, technical, and medical journals.

Experimental Methods for Identification of Genes Involved in the Action of a Drug

There are a number of experimental methods for identifying genes and gene products that mediate or modulate the effects of a drug or other treatment. They encompass analyses of RNA and protein expression as well as methods for detecting protein—protein interactions and protein—ligand interactions. Two preferred experimental methods for identification of genes that may be involved in the action of a drug are (1) methods for measuring the expression levels of many mRNA transcripts in cells or organisms treated with the drug (2) methods for measuring the expression levels of many proteins in cells or organisms treated with the drug.

RNA transcripts or proteins that are substantially increased or decreased in drug treated cells or tissues relative to control cells or tissues are candidates for mediating the action of the drug. Preferably the level of an mRNA is at least 30% higher or lower in drug treated cells, more preferably at least 50% higher or lower, and most preferably two fold higher or lower than levels in non-drug treated control cells. The analysis of RNA levels can be performed on total RNA or on polyadenylated RNA selected by oligodT affinity. Further, RNA from different cell compartments can be analyzed independently—for example nuclear vs. cytoplasmic RNA. In addition to RNA levels, RNA kinetics can be examined, or the pool of RNAs currently being translated can be analyzed by isolation of RNA from polysomes. Other useful experimental methods include protein interaction methods such as the yeast two hybrid system and variants thereof which facilitate the detection of protein—protein interactions. Preferably one of the interacting proteins is the drug target or another protein strongly implicated in the action of the compound being assessed.

The pool of RNAs expressed in a cell is sometimes referred to as the transcriptome. Methods for measuring the transcriptome, or some part of it, are known in the art. A recent collection of articles summarizing some current methods appeared as a supplement to the journal Nature Genetics. (The Chipping Forecast. Nature Genetics supplement, volume 21, January 1999.) A preferred method for measuring expression levels of mRNAs is to spot PCR products corresponding to a large number of specific genes on a nylon membrane such as Hybond N Plus (Amersham-Pharmacia). Total cellular mRNA is then isolated, labeled by random oligonucleotide priming in the presence of a detectable label (e.g. alpha 33P labeled radionucleotides or dye labeled nucleotides), and hybridized with the filter containing the PCR products. The resulting signals can be analyzed by commercially available software, such as can be obtained from Clontech/Molecular Dynamics or Research Genetics, Inc.

Experiments have been described in model systems that demonstrate the utility of measuring changes in the transcriptome before and after changing the growth conditions of cells, for example by changing the nutrient environment. The changes in gene expression help reveal the network of genes that mediate physiological responses to the altered growth condition. Similarly, the addition of a drug to the cellular or in vivo environment, followed by monitoring the changes in gene expression can aid in identification of gene networks that mediate pharmacological responses.

The pool of proteins expressed in a cell is sometimes referred to as the proteome. Studies of the proteome may include not only protein abundance but also protein subcellular localization and protein-protein interaction. Methods for measuring the proteome, or some part of it, are known in the art. One widely used method is to extract total cellular protein and separate it in two dimensions, for example first by size and then by isoelectric point. The resulting protein spots can be stained and quantitated, and individual spots can be excised and analyzed by mass spectrometry to provide definitive identification. The results can be compared from two or more cell lines or tissues, at least one of which has been treated with a drug. The differential up or down modulation of specific proteins in response to drug treatment may indicate their role in mediating the pharmacologic actions of the drug. Another way to identify the network of proteins that mediate the actions of a drug is to exploit methods for identifying interacting proteins. By starting with a protein known to be involved in the action of a drug—for example the drug target—one can use systems such as the yeast two hybrid system and variants thereof (known to those skilled in the art; see Ausubel et al., Current Protocols in Molecular Biology, op. cit.) to identify additional proteins in the network of proteins that mediate drug action. The genes encoding such proteins would be useful for screening for DNA sequence variances, which in turn may be useful for analysis of interpatient variation in response to treatments. For example, the protein 5-lipoxygenase (5LO) is an enzyme which is at the beginning of the leukotriene biosynthetic pathway and is a target for anti-inflammatory drugs used to treat asthma and other diseases. In order to detect proteins that interact with 5-lipoxygenase the two-hybrid system was recently used to isolate three different proteins, none previously known to interact with 5LO. (Provost et al., Interaction of 5-lipoxygenase with cellular proteins. Proc. Natl. Acad. Sci. U.S.A. 96: 1881-1885, 1999.) A recent collection of articles summarizing some current methods in proteomics appeared in the August 1998 issue of the journal Electrophoresis (volume 19, number 11). Other useful articles include: Blackstock W P), et al. Proteomics: quantitative and physical mapping of cellular proteins. Trends Biotechnol. 17 (3): p. 121-7, 1999, and Patton W. F., Proteome analysis II. Protein subcellular redistribution: linking physiology to genomics via the proteome and separation technologies involved. J Chromatogr B Biomed Sci App. 722(1-2):203-23. 1999.

Since many of these methods can also be used to assess whether specific polymorphisms are likely to have biological effects, they are also relevant in section 3, below, concerning methods for assessing the likely contribution of variances in candidate genes to clinical variation in patient responses to therapy.

2. Screen for Variances in Genes that may be Related to Therapeutic Response

Having identified a set of genes that may affect response to a drug the next step is to screen the genes for variances that may account for interindividual variation in response to the drug. There are a variety of levels at which a gene can be screened for variances, and a variety of methods for variance screening. The two main levels of variance screening are genomic DNA screening and cDNA screening. Genomic variance detection may include screening the entire genomic segment spanning the gene from 2 kb to 10 kb upstream of the transcription start site to the polyadenylation site, or 2 to 10 kb beyond the polyadenylation site. Alternatively genomic variance detection may (for intron containing genes) include the exons and some region around them containing the splicing signals, for example, but not all of the intronic sequences. In addition to screening introns and exons for variances it is generally desirable to screen regulatory DNA sequences for variances. Promoter, enhancer, silencer and other regulatory elements have been described in human genes. The promoter is generally proximal to the transcription start site, although there may be several promoters and several transcription start sites. Enhancer, silencer and other regulatory elements may be intragenic or may lie outside the introns and exons, possibly at a considerable distance, such as 100 kb away. Variances in such sequences may affect basal gene expression or regulation of gene expression. In either case such variation may affect the response of an individual patient to a therapeutic intervention, for example a drug, as described in the examples. Thus in practicing the present invention it is useful to screen regulatory sequences as well as transcribed sequences, in order to identify variances that may affect gene transcription. Frequently the genomic sequence of a gene can be found in the sources above, particularly by searching GenBank or Medline (PubMed). The name of the gene can be entered at a site such as Entrez: http://www.ncbi.nlm.nih.gov/Entrez/nucleotide.html. Using the genomic sequence and information from the biomedical literature one skilled in the art can perform a variance detection procedure such as those described in examples 15, 16 and 17.

Variance detection is often first performed on the cDNA of a gene for several reasons. First, available data on functional sequence variances suggests that variances in the transcribed portion of a gene may be most likely to have functional consequences as they can affect the interaction of the transcript with a wide variety of cellular factors during the complex processes of RNA transcription, processing and translation, with consequent effects on RNA splicing, stability, translational efficiency or other processes. Second, as a practical matter the cDNA sequence of a gene is often available before the genomic structure is known, although the reverse will be true in the future as the sequence of the human genome is determined. Third, the cDNA is often compact compared to the genomic locus, and can be screened for variances with much less effort. If the genomic structure is not known then only the cDNA sequence can be scanned for variances. Methods for preparing cDNA are described in Example 14. Methods for variance detection on cDNA are described below and in the examples.

In general it is preferable to catalog genetic variation at the genomic DNA level because there are an increasing number of well documented instances of functionally important variances that lie outside of transcribed sequence. Also, to properly use optimal genetic methods to assess the contribution of a candidate gene to variation in a phenotype of interest it is desirable to understand the character of sequence variation in the candidate gene: what is the nature of linkage disequilibrium between different variances in the gene; are there sites of recombination within the gene; what is the extent of homoplasy in the gene (i.e. occurrence of two variant sites that are identical by state but not identical by descent because the same variance arose at least twice in human evolutionary history on two different haplotypes); what are the different haplotypes and how can they be grouped to increase the power of genetic analysis?

Methods for variance screening have been described, including DNA sequencing. See for example: U.S. Pat. No. 5,698,400: Detection of mutation by resolvase cleavage; U.S. Pat. No. 5,217,863: Detection of mutations in nucleic acids; and U.S. Pat. No. 5,750,335: Screening for genetic variation, as well as the examples and references cited therein for examples of useful variance detection procedures. Detailed variance detection procedures are also described in examples 15, 16 and 17. One skilled in the art will recognize that depending on the specific aims of a variance detection project (number of genes being screened, number of individuals being screened, total length of DNA being screened) one of the above cited methods may be preferable to the others, or yet another procedure may be optimal. A preferred method of variance detection is chain terminating DNA sequencing using dye labeled primers, cycle sequencing and software for assessing the quality of the DNA sequence as well as specialized software for calling heterozygotes. The use of such procedures has been described by Nickerson and colleagues. See for example: Rieder M. J., et al. Automating the identification of DNA variations using quality-based fluorescence re-sequencing: analysis of the human mitochondrial genome. Nucleic Acids Res. 26 (4):967-73, 1998, and: Nickerson D. A., et al. PolyPhred: automating the detection and genotyping of single nucleotide substitutions using fluorescence-based resequencing. Nucleic Acids Res. 25 (14):2745-51, 1997.Although the variances provided in Tables 3, and 4 consist principally of cDNA variances, it is an aspect of this invention that detection of genomic variances is also a useful method for identification of variances that may account for interpatient variation in response to a therapy.

Another important aspect of variance detection is the use of DNA from a panel of human subjects that represents a known population. For example, if the subjects are being screened for variances relevant to a specific drug development program it is desirable to include both subjects with the target disease and healthy subjects in the panel, because certain variances may occur at different frequencies in the healthy and disease populations and can only be reliably detected by screening both populations. Also, for example, if the drug development program is taking place in Japan, it is important to include Japanese individuals in the screening population. In general, it is always desirable to include subjects of known geographic, racial or ethnic identity in a variance screening experiment so the results can be interpreted appropriately for different patient populations, if necessary. Also, in order to select optimal sets of variances for genetic analysis of a gene locus it is desirable to know which variances have occurred recently—perhaps on multiple different chromosomes—and which are ancient. Inclusion of one or more apes or monkeys in the variance screening panel is one way of gaining insight into the evolutionary history of variances. Chimpanzees are preferred subjects for inclusion in a variance screening panel.

3. Assess the Likely Contribution of Variances in Candidate Genes to Clinical Variation in Patient Responses to Therapy

Once a set of genes likely to affect disease pathophysiology or drug action has been identified, and those genes have been screened for variances, said variances (e.g., provided in Tables 3, and 4) can be assessed for their contribution to variation in the pharmacological or toxicological phenotypes of interest. Such studies are useful for reducing a large number of candidate variances to a smaller number of variances to be tested in clinical trials. There are several methods which can be used in the present invention for assessing the medical and pharmaceutical implications of a DNA sequence variance. They range from computational methods to in vitro and/or in vivo experimental methods, to prospective human clinical trials, and also include a variety of other laboratory and clinical measures that can provide evidence of the medical consequences of a variance. In general, human clinical trials constitute the highest standard of proof that a variance or set of variances is useful for selecting a method of treatment, however, computational and in vitro data, or retrospective analysis of human clinical data may provide strong evidence that a particular variance will affect response to a given therapy, often at lower cost and in less time than a prospective clinical trial. Moreover, at an early stage in the analysis when there are many possible hypotheses to explain interpatient variation in treatment response, the use of informatics-based approaches to evaluate the likely functional effects of specific variances is an efficient way to proceed.

Informatics-based approaches to the prediction of the likely functional effects of variances include DNA and protein sequence analysis (phylogenetic approaches and motif searching) and protein modeling (based on coordinates in the protein database, or pdb; see http://www.rcsb.org/pdb/). See, for example: Kawabata et al. The Protein Mutant Database. Nucleic Acids Research 27: 355-357, 1999; also available at: http://pmd.ddbj.nig.ac.ip. Such analyses can be performed quickly and inexpensively, and the results may allow selection of certain genes for more extensive in vitro or in vivo studies or for more variance detection or both.

The three dimensional structure of many medically and pharmaceutically important proteins, or homologs of such proteins in other species, or examples of domains present in such proteins, is known as a result of x-ray crystallography studies and, increasingly, nuclear magnetic resonance studies. Further, there are increasingly powerful tools for modeling the structure of proteins with unsolved structure, particularly if there is a related (homologous) protein with known structure. (For reviews see: Rost et al., Protein fold recognition by prediction-based threading, J. Mol. Biol. 270:471-480, 1997; Firestine et al., Threading your way to protein function, Chem. Biol. 3:779-783, 1996) There are also powerful methods for identifying conserved domains and vital amino acid residues of proteins of unknown structure by analysis of phylogenetic relationships. (Deleage et al., Protein structure prediction: Implications for the biologist, Biochimie 79:681-686, 1997; Taylor et al., Multiple protein structure alignment, Protein Sci. 3:1858-1870, 1994) These methods can permit the prediction of functionally important variances, either on the basis of structure or evolutionary conservation. For example, a crystal structure can reveal which amino acids comprise a small molecule binding site. The identification of a polymorphic amino acid variance in the topological neighborhood of such a site, and, in particular, the demonstration that at least one variant form of the protein has a variant amino acid which impinges on (or which may otherwise affect the chemical environment around) the small molecule binding pocket differently from another variant form, provides strong evidence that the variance may affect the function of the protein. From this it follows that the interaction of the protein with a treatment method, such an administered compound, will likely be variable between different patients. One skilled in the art will recognize that the application of computational tools to the identification of functionally consequential variances involves applying the knowledge and tools of medicinal chemistry and physiology to the analysis.

Phylogenetic approaches to understanding sequence variation are also useful. Thus if a sequence variance occurs at a nucleotide or encoded amino acid residue where there is usually little or no variation in homologs of the protein of interest from non-human species, particularly evolutionarily remote species, then the variance is more likely to affect function of the RNA or protein. Computational methods for phylogenetic analysis are known in the art, (see below for citations of some methods).

Computational methods are also useful for analyzing DNA polymorphisms in transcriptional regulatory sequences, including promoters and enhancers. One useful approach is to compare variances in potential or proven transcriptional regulatory sequences to a catalog of all known transcriptional regulatory sequences, including consensus binding domains for all transcription factor binding domains. See, for example, the databases cited in: Burks, C. Molecular Biology Database List. Nucleic Acids Research 27: 1-9, 1999, and links to useful databases on the internet at: http://www.oup.co.uk/nar/Volume_{—}27/issue_{—}01/summary/gkc105_gml.html. In particular see the Transcription Factor Database (Heinemeyer, T., et al. (1999) Expanding the TRANSFAC database towards an expert system of regulatory molecular mechanisms. Nucleic Acids Res. 27: 318-322, or on the internet at: http://193.175.244.40/TRANSFAC/index.html). Any sequence variances in transcriptional regulatory sequences can be assessed for their effects on mRNA levels using standard methods, either by making plasmid constructs with the different allelic forms of the sequence, transfecting them into cells and measuring the output of a reporter transcript, or by assays of cells with different endogenous alleles of variances. One example of a polymorphism in a transcriptional regulatory element that has a pharmacogenetic effect is described by Drazen et al. (1999) Pharmacogenetic association between ALOX5 promoter genotype and the response to anti-asthma treatment. Nature Genetics 22: 168-170. Drazen and co-workers found that a polymorphism in an Sp1-transcription factor binding domain, which varied among subjects from 3-6 tandem copies, accounted for varied expression levels of the 5-lipoxygenase gene when assayed in vitro in reporter construct assays. This effect would have been flagged by an informatics analysis that surveyed the 5-lipoxygenase candidate promoter region for transcriptional regulatory sequences (resulting in discovery of polymorphism in the Sp1 motif).

4. Perform in vitro or in vivo Experiments to Assess the Functional Importance of Gene Variances

There are two broad types of studies useful for assessing the likely importance of variances: analysis of RNA or protein abundance (as described above in the context of methods for identifying candidate genes for explaining interpatient variation in treatment response) or analysis of functional differences in different variant forms of a gene, mRNA or protein. Studies of functional differences may involve direct measurements of biochemical activity of different variant forms of an mRNA or protein, or may involve assaying the influence of a variance or variances on various cell properties, including both tissue culture and in vivo studies.

The selection of an appropriate experimental program for testing the medical consequences of a variance may differ depending on the nature of the variance, the gene, and the disease. For example if there is already evidence that a protein is involved in the pharmacologic action of a drug, then the in vitro or in vivo demonstration that an amino acid variance in the protein affects its biochemical activity is strong evidence that the variance will have an effect on the pharmacology of the drug in patients, and therefore that patients with different variant forms of the gene may have different responses to the same dose of drug. If the variance is silent with respect to protein coding information, or if it lies in a noncoding portion of the gene (e.g., a promoter, an intron, or a 5′- or 3′-untranslated region) then the appropriate biochemical assay may be to assess mRNA abundance, half life, or translational efficiency. If, on the other hand, there is no substantial evidence that the protein encoded by a particular gene is relevant to drug pharmacology, but instead is a candidate gene on account of its involvement in disease pathophysiology, then the optimal test may be a clinical study addressing whether two patient groups distinguished on the basis of the variance respond differently to a therapeutic intervention. This approach reflects the current reality that biologists do not sufficiently understand gene regulation, gene expression and gene function to consistently make accurate inferences about the consequences of DNA sequence variances for pharmacological responses.

In summary, if there is a plausible hypothesis regarding the effect of a protein on the action of a drug, then in vitro and in vivo approaches, including those described below, will be useful to predict whether a given variance is therapeutically consequential. If, on the other hand, there is no evidence of such an effect, then the preferred test is an empirical clinical measure of the impact to the variance on efficacy or toxicity in vivo (which requires no evidence or assumptions regarding the mechanism by which the variance may exert an effect on a therapeutic response). However, given the expense and statistical constraints of clinical trials, it is preferable to limit clinical testing to variances for which there is at least some experimental or computational evidence of a functional effect.

In another aspect of the invention a powerful, high throughput approach to the genetics of drug response is to study variation in drug response phenotypes among cell lines derived from related individuals. Consider a cellular drug response phenotype that is readily measured, and that varies among cell lines. The demonstration of Mendelian transmission of the drug response phenotype in cell lines from related individuals would constitute evidence of a genetic component to the drug response phenotype. The expected pattern of segregation depends on making an assumption about the genetic model: recessive, dominant or co-dominant alleles will produce different proportions in the progeny of a cross. The value of studying cell lines as surrogates for people is that experiments can be performed for a small fraction of the cost. The value of studying cell lines from related individuals is that genetic effects on drug response are likely to be much easier to identify when genetic background among the subjects is substantially similar. In particular, in cell lines from a pedigree it is known that only four parental alleles are segregating in the children, and that any two children are on average 50% genetically identical. In a more heterogeneous genetic background (i.e. cell lines from unrelated subjects) the effect of allelic variation at multiple genes that modulate the measured drug response phenotypes is more likely to create a nearly continuous distribution of responses (except in cases where the product of one gene accounts for most of the measured drug response phenotype).

Many cell lines have been derived from groups of related individuals, or pedigrees. A commercial source of such cell lines is the Human Genetic Cell Respository, supported by the National Institute of General Medical Sciences (NIGMS) and housed at the Coriell Cell Repository, Camden, N.J. A directory of these cell lines is available on the world wide web: http://locus.umdnj.edu/nigms/. One preferred set of cell lines for pharmacogenetic studies, available from the Coriell Cell Repository, is the set of cell lines used by the Centre d'Etudes du Polymorphisme Humain (CEPH) consortium (Paris, France) to establish a detailed genetic map of man. See, for example: Gyapay, G., Morissette, J., Vignal, A., et al. (1994) The 1993-94 Genethon human genetic linkage map. Nature Genetics 7(2 Spec No):246-339. More current data on the CEPH genetic linkage map can be found on the world wide web at: http://landru.cephb.fr/cephdb/. Lymphoblastoid cell lines from 57 CEPH families are available from the Coriell Repository. In most cases the families consist of four grandparents, two parents and between four and twelve children.

The principal attraction of the CEPH cell lines for pharmacogenetic studies is that a detailed genetic map of nearly 12,000 polymorphic markers has been established via an international effort, and the map data are freely available on the world wide web. In other words the genotypes of thousands of polymorphic markers are known in most of the CEPH cell lines (not all markers were studied in all cell lines). As a result, one skilled in the art can determine the chromosomal location of any locus that controls a Mendelian trait in these cell lines, using software for linkage analysis such as the programs LINKAGE, CRIMAP and MAPMAKER. (See, for example: Lander, E. S., Green, P., Abrahamson, J., et al. (1987) MAPMAKER: an interactive computer package for constructing primary genetic linkage maps of experimental and natural populations. Genomics 1(2): 174-81. See also: Terwilliger, J. and J. Ott (1994), Handbook of Human Linkage Analysis. John Hopkins University Press, Baltimore for a more exhaustive description of linkage analysis methods.)

One set of interesting Mendelian traits to study using the CEPH cell lines (or similar cell lines from pedigrees) and the genetic approach just described are drug response phenotypes. Consider, for example, a G protein coupled receptor that exists in two allelic forms that behave differently in the presence of a compound being developed for human clinical use (e.g. one receptor binds the compound with higher affinity than the other). Methods for assaying G protein mediated signal transduction are well known in the art. By adding the compound (either at a fixed concentration or at a series of different concentrations) to a family-derived set of lymphoblastoid cell lines (which of course must express the G protein coupled receptor) and measuring the signal produced it should be possible to detect the segregation of the grandparental alleles in the parents and the segregation of parental alleles in the children. For example, consider two alleles of the receptor: if allele A produces a greater signal than allele B at a given concentration of the compound, and if one parent is an AB heterozygote while the other parent is a BB heterozygote then the levels of signal in the children should be medium (in AB heterozygotes) or low (in BB homozygotes). The detection of such a pattern in cell lines of the family would constitute evidence that the G protein coupled receptor polymorphism was responsible for intersubject differences in response to the compound. (More generally, the detection of any discrete partitioning of responses in the data—high and low, or high medium and low—is suggestive of genetic control, with the genetic model to be inferred from the pattern of inheritance, and support for the hypothesis to come from the analysis of multiple families.) It is not necessary to know the identify of the variant gene in advance (as in the G protein coupled receptor example just provided). The pattern of segregation of the drug response phenotype in the cell lines of the various members of the CEPH families can be compared to the pattern of segregation of the thousands of polymorphic markers already typed in the same cell lines.

Those polymorphic markers that co-segregate with the drug response phenotype are candidates for marking the location of the locus responsible for the drug response phenotype. By performing the same experiment in cell lines from multiple (e.g. from two up to 57 CEPH) families the list of candidate polymorphic markers generally narrows to a few, all of which (or nearly all of which) are from the same chromosomal region—viz. the region harboring the gene responsible for the drug response phenotype. Knowing (i) the chromosomal location of the gene (or genes) implicated by the linkage analysis, together with (ii) information about the location and function of genes in that chromosomal region (available from online databases, for example, those at the U.S. National Center for Biotechnology Information; see http://www.ncbi.nlm.nih.gov/LocusLink/), and further (iii) knowing something of the pharmacology of the compound and consequently the metabolic and regulatory pathways likely to influence its action, should constrain the list of candidate genes likely to be responsible for the observed variation to a small number of genes. These genes (if there is more than one) can be systematically evaluated for pharmacogenetic impact by identifying polymorphisms and testing whether they cosegregate with drug response phenotypes in the pedigrees, in new pedigrees, in cells from unrelated individuals, or in vivo in a population of nonrelated individuals, for example in a clinical trial.

Some drug response phenotypes may not behave as Mendelian traits, but may rather be continuous (quantitative) traits under the control of several genes. Variation at any of the relevant gene loci could affect drug response, often to different extents. Robust methods for mapping quantitative trait loci (QTL) are known in the art. For example, see: Shugart, Y. Y. and Goldgar, D. E. (1999) Multipoint genomic scanning for quantitative loci: effects of map density, sibship size and computational approach. Eur J Hum Genet 7(2):103-9. It is worth emphasizing that in the approach described (using the CEPH cell lines) there is no need for genotyping in order to map the drug response traits in the cell lines; the effort already expended to produce a human linkage map in the CEPH cell lines can be exploited.

Cell responses that could be usefully characterized by the above methods include for example the level of signaling in a pathway that mediates the response to a compound (as in the G protein coupled receptor assays where levels of a second messenger are measured), compound uptake, compound metabolism, levels of metabolites affected by a compound, levels of proteins (including enzymes in biochemical pathways related to the action of the compound), levels of an inhibitory complex formed by a compound, and other assays known to those skilled in the art of pharmacology and assay development. For example, a study of the genetic basis of variation in response to the antineoplastic drug 5-fluorouracil might include measurement of cell uptake of 5-FU, conversion of 5-FU to inactive metabolites such as 5,6-dihydrofluorouridine or fluoro-beta alanine, conversion of 5-FU to active metabolites such as 5-fluorodeoxyuridine, levels of thymidylate synthetase (an enzyme inhibited by 5-FU), levels of 5, 10 methylenetetrahydrofolate (a folate co-factor essential for 5-FU mediated inhibition of thymidylate synthetase) and the enzymes that produce it, or levels of nucleotide pools or the enzymes that produce them. All of the relevant transporters and enzymes are expressed in lymphoblastoid cells, even though 5-FU is not routinely used in the therapy of lymphoid malignancies.

However, a limitation of lymphoblastoid cell lines for the methods described above is that they are not suitable for all of the different types of assays one might wish to perform. One alternative is to use fibroblast cell lines, which have already been derived from multiple different families. Fibroblasts are not available from the CEPH pedigrees, however a set of fibroblasts from known pedigrees could be genotyped at a set of highly polymorphic markers to produce a genetic map. Another approach is to treat lymphoblastoid cells with a procedure or agent that induces differentiation to a different cell type, such as an adipocye or a myocyte. For example, there are genes which effectively control differentiation programs (e.g. peroxisome proliferator activated receptor [PPAR] gamma mediates adipocyte differentiation, myoD mediates myocyte differentiation); introduction of such a gene into a cell line of one type can alter its differentiated state to another cell type. Alternatively, stimulation of the gene product of such a regulatory gene (e.g. treatment of cells with the PPAR gamma agonist troglitazone) can be used to induce differentiation to a different cell type. Such procedures are known in the art, and may be effectively applied to human lymphoblasts.

In preferred embodiments of the above methods the cells used are from the CEPH pedigrees. Preferably at least one pedigree is studied, more preferably two pedigrees, still more preferably five pedigrees and most preferably eight pedigrees or more. It is useful to perform a statistical calculation to determine how many pedigrees and cell lines should be studied to achieve a given power to detect an effect, making assumptions about the magnitude of the effect.

In another aspect, described below, the methods described above can be used to identify mRNAs that vary in levels between cell lines as a result of genetically controlled regulatory factors, such as, for example, polymorphisms in promoters that affect the binding or action of transcriptional regulatory factors. Such variation in mRNA levels may be responsible for intersubject variation in drug response.

Experimental Methods: Genomic DNA Analysis

Variances in DNA may affect the basal transcription or regulated transcription of a gene locus. Such variances may be located in any part of the gene but are most likely to be located in the promoter region, the first intron, or in 5′ or 3′ flanking DNA, where enhancer or silencer elements may be located. Methods for analyzing transcription are well known to those skilled in the art and exemplary methods are briefly described above and in some of the texts cited elsewhere in this application. Transcriptional run off assay is one useful method. Detailed protocols can be found in texts such as: Current Protocols in Molecular Biology edited by: F. M. Ausubel, et al. John Wiley & Sons, Inc, 1999, or: Molecular Cloning: A Laboratory Manual by J. Sambrook, E. F. Fritsch and T Maniatis. 1989. 3 vols, 2nd edition, Cold Spring Harbor Laboratory Press

Experimental Methods: RNA Analysis

RNA variances may affect a wide range of processes including RNA splicing, polyadenylation, capping, export from the nucleus, interaction with translation initiation, elongation or termination factors, or the ribosome, or interaction with cellular factors including regulatory proteins, or factors that may affect mRNA half life. However, the effect of most RNA sequence variances on RNA function, if any, should ultimately be measurable as an effect on RNA or protein levels—either basal levels or regulated levels or levels in some abnormal cell state, such as cells from patients with a disease. Therefore, one preferred method for assessing the effect of RNA variances on RNA function is to measure the levels of RNA produced by different alleles in one or more conditions of cell or tissue growth. Said measuring can be done by conventional methods such as Northern blots or RNAase protection assays (kits available from Ambion, Inc.), or by methods such as the Taqman assay (developed by the Applied Biosystems Division of the Perkin Elmer Corporation), or by using arrays of oligonucleotides or arrays of cDNAs attached to solid surfaces. Systems for arraying cDNAs are available commercially from companies such as Nanogen and General Scanning. Complete systems for gene expression analysis are available from companies such as Molecular Dynamics. For recent reviews of systems for high throughput RNA expression analysis see the supplement to volume 21 of Nature Genetics entitled “The Chipping Forecast”, especially articles beginning on pages 9, 15, 20 and 25.

Additional methods for analyzing the effect of variances on RNA include secondary structure probing, and direct measurement of half life or turnover. Secondary structure can be determined by techniques such as enzymatic probing (using enzymes such as T1, T2 and S1 nuclease), chemical probing or RNAase H probing using oligonucleotides. Most RNA structural assays are performed in vitro, however some techniques can be performed on cell extracts or even in living cells, using fluorescence resonance energy transfer to monitor the state of RNA probe molecules.

In another aspect the methods described above (relating to the use of cell lines from pedigrees to genetically map phenotypes that can be studied in tissue culture cells) can be used to identify mRNAs that vary in levels between individuals as a result of genetically controlled factors. Genetic factors include both cis-acting polymorphisms, such as might be present in promoters (e.g. polymorphisms that affect the binding or action of transcription factors) as well as trans-acting factors such as might be present in transcription factors (e.g. an amino acid polymorphism that affects the interaction of a transcription factor with a promoter element, or that might affect levels of the transcription factor itself). Variation in mRNA levels may contribute to intersubject variation in drug response, disease susceptibility or disease manifestations. (See above for example of promoter polymorphism in 5-lipoxygenase and its effect on response to anti-asthma medications.)

The methods for identifying mRNAs which vary in abundance as a consequence of genetic mechanisms are similar to those described above for drug response phenotypes. First, by examining whether levels of an mRNA segregate in one or more pedigrees it is possible to infer whether there is a genetic component to the variation. Second, by inspecting the CEPH genotype data it is possible to identify genetic markers that cosegregate with the mRNA expression levels (either increased or decreased) and thereby map the chromosomal location of the locus or loci that control mRNA levels. Third, by inspection of the genes at the chromosomal locus controlling mRNA levels it should be possible to identify one or a few genes that are likely responsible for the effect. These genes can then be definitively evaluated by discovering variances and testing if they predict mRNA levels (or other phenotypes) in the pedigree cell lines, in cell lines from unrelated individuals, or in vivo. Fourth, the above analysis can be performed on cell lines subjected to various pharmacological or nutritional manipulations. For example cell lines from one or more pedigrees can be treated with a drug, or deprived of an amino acid and mRNA levels measured at various times after treatment. Any variable differences in mRNA levels in response to the treatment, if they segregate in pedigrees, can be subjected to steps 1-3. Fifth, this analysis can be performed at very large scale using arrays of gridded cDNAs, PCR products or oligonucleotides corresponding to an unlimited number of genes. In each experiment the RNA from the pedigree cell lines (treated or not) is isolated, labeled using standard methods and hybridized to the grids containing the nucleic acids corresponding to the genes being investigated. Current commercial methods permit up to 400,000 oligonucleotides (more than the total number of human genes) to be queried in one experiment, although lower density formats are also well suited to the methods described. Thus, in a comparatively modest number of experiments the entire transcript population of lymphoblasts (probably <25,000 unique transcripts) can be queried for genetically controlled variation in mRNA abundance. Other types of cell lines can be subjected to similar analysis.

The variation in mRNA levels due to gene polymorphisms is likely to be of small magnitude (generally two-fold differences or less are expected). Therefore a key aspect of experimental systems used to measure mRNA levels is their accuracy. Preferably a system capable of resolving mRNAs that differ in abundance (measured in molecules per cell, or relative to a standard such as total mRNA or one or more specific RNAs such as actin or clathrin or glucose-6-phosphare dehydrogenase) is sufficiently sensitive to detect differences as small as 50%, more preferably as small as 30%, and most preferably as small as 20%.

There are 757 individuals in the 57 CEPH cell lines. Thus all the CEPH cell lines could fit in eight 96 well microtiter plates. Microtiter plates provide a convenient format for growing cells and for performing cell manipulations, such as those described above, using multichannel pipettes or automated pipetting robots. By growing cells in large volume flasks, counting them (by hemocytometer or Coulter counter or other means) and then aliquoting them robotically to 96 well plates it is possible to assure that each well has nearly the same number of cells. A large number of plates can be prepared in this way and then stored frozen in appropriate medium until needed for experiments.

Experimental Methods: Protein Analysis

There are a variety of experimental methods for investigating the effect of an amino acid variance on response of a patient to a treatment. The preferred method will depend on the availability of cells expressing a particular protein, and the feasibility of a cell-based assay vs. assays on cell extracts, on proteins produced in a foreign host, or on proteins prepared by in vitro translation.

For example, the methods and systems listed below can be utilized to demonstrate differential expression, stability and/or activity of different variant forms of a protein, or in phenotype/genotype correlations in a model system.

For the determination of protein levels or protein activity a variety of techniques are available. The in vitro protein activity can be determined by transcription or translation in bacteria, yeast, baculovirus, COS cells (transient), Chinese Hamster Ovary (CHO) cells, or studied directly in human cells, or other cell systems can be used. Further, one can perform pulse chase experiments to determine if there are changes in protein stability (half-life).

One skilled in the art can construct cell based assays of protein function, and then perform the assays in cells with different genotypes or haplotypes. For example, identification of cells with different genotypes, e.g., cell lines established from families and subsequent determination of relevant protein phenotypes (e.g., expression levels, post translational modifications, activity assays) may be performed using standard methods.

Assays of protein levels or function can also be performed on cell lines (or extracts from cell lines) derived from pedigrees in order to determine whether there is a genetic component to variation in protein levels or function. The experimental analysis is as above for RNAs, except the assays are different. Experiments can be performed on naive cells or on cells subjected to various treatments, including pharmacological treatments.

In another approach to the study of amino acid variances one can express genes corresponding to different alleles in experimental organisms and examine effects on disease phenotype (if relevant in the animal model), or on response to the presence of a compound. Such experiments may be performed in animals that have disrupted copies of the homologous gene (e.g. gene knockout animals engineered to be deficient in a target gene), or variant forms of the human gene may be introduced into germ cells by transgenic methods, or a combination of approaches may be used. To create animal strains with targeted gene disruptions a DNA construct is created (using DNA sequence information from the host animal) that will undergo homologous recombination when inserted into the nucleus of an embryonic stem cell. The targeted gene is effectively inactivated due to the insertion of non-natural sequence—for example a translation stop codon or a marker gene sequence that interrupts the reading frame. Well known PCR based methods are then used to screen for those cells in which the desired homologous recombination event has occurred. Gene knockouts can be accomplished in worms, drosophila, mice or other organisms. Once the knockout cells are created (in whatever species) the candidate therapeutic intervention can be administered to the animal and pharmacological or biological responses measured, including gene expression levels. If variant forms of the gene are useful in explaining interpatient variation in response to the compound in man, then complete absence of the gene in an experimental organism should have a major effect on drug response. As a next step various human forms of the gene can be introduced into the knockout organism (a technique sometimes referred to as a knock-in). Again, pharmacological studies can be performed to assess the impact of different human variances on drug response. Methods relevant to the experimental approaches described above can be found in the following exemplary texts:

General Molecular Biology Methods

Molecular Biology: A project approach, S. J. Karcher, Fall 1995. Academic Press

DNA Cloning: A Practical Approach, D. M. Glover and B. D. Hayes (eds). 1995. IRL/Oxford University Press. Vol. 1—Core Techniques; Vol 2—Expression Systems; Vol. 3—Complex Genomes; Vol. 4—Mammalian Systems.

Short Protocols in Molecular Biology, Ausubel et al. October 1995. 3rd edition, John Wiley and Sons

Current Protocols in Molecular Biology Edited by: F. M. Ausubel, R. Brent, R. E. Kingston, D. D. Moore, J. G. Seidman, K. Struhl, (Series Editor: V. B. Chanda), 1988

Molecular Cloning: A laboratory manual, J. Sambrook, E. F. Fritsch. 1989. 3 vols, 2nd edition, Cold Spring Harbor Laboratory Press

Polymerase Chain Reaction (PCR)

PCR Primer: A laboratory manual, C. W. Diffenbach and G. S. Dveksler (eds.). 1995. Cold Spring Harbor Laboratory Press.

The Polymerase Chain Reaction, K. B. Mullis et al. (eds.), 1994. Birkhauser

PCR Strategies, M. A. Innis, D. H. Gelf, and J. J. Sninsky (eds.), 1995. Academic Press

General Procedures for Discipline Specific Studies

Current Protocols in Neuroscience Edited by: J. Crawley, C. Gerfen, R. McKay, M. Rogawski, D. Sibley, P. Skolnick, (Series Editor: G. Taylor), 1997.

Current Protocols in Pharmacology Edited by: S. J. Enna/M. Williams, J. W. Ferkany, T. Kenakin, R. E. Porsolt, J. P. Sullivan, (Series Editor: G. Taylor),1998.

Current Protocols in Protein Science Edited by: J. E. Coligan, B. M. Dunn, H. L. Ploegh, D. W. Speicher, P. T. Wingfield, (Series Editor: Virginia Benson Chanda), 1995.

Current Protocols in Cell Biology Edited by: J. S. Bonifacino, M. Dasso, J. Lippincott-Schwartz, J. B. Harford, K. M. Yamada, (Series Editor: K. Morgan) 1999.

Current Protocols in Cytometry Managing Editor: J. P. Robinson, Z. Darzynkiewicz (ed)/P. Dean (ed), A. Orfao (ed), P. Rabinovitch (ed), C. Stewart (ed), H. Tanke (ed), L. Wheeless (ed), (Series Editor: J. Paul Robinson), 1997.

Current Protocols in Human Genetics Edited by: N. C. Dracopoli, J. L. Haines, B. R. Korf, et al., (Series Editor: A. Boyle), 1994.

Current Protocols in Immunology Edited by: J. E. Coligan, A. M. Kruisbeek, D. H. Margulies, E. M. Shevach, W. Strober, (Series Editor: R. Coico), 1991.

IV. Clinical Trials

A clinical trial is the definitive test of the utility of a variance or variances for the selection of optimal therapy. A clinical trial in which an interaction of gene variances and clinical outcomes (desired or undesired) is explored will be referred to herein as a “pharmacogenetic clinical trial”. Pharmacogenetic clinical trials require no knowledge of the biological function of the gene containing the variance or variances to be assessed, nor any knowledge of how the therapeutic intervention to be assessed works at a biochemical level. The pharmacogenetics effects of a variance can be addressed at a purely statistical level: either a particular variance or set of variances is consistently associated with a significant difference in a salient drug response parameter (e.g. response rate, effective dose, side effect rate, etc.) or not. On the other hand, if there is information about either the biochemical basis of a therapeutic intervention or the biochemical effects of a variance, then a pharmacogenetic clinical trial can be designed to test a specific hypothesis. In preferred embodiments of the methods of this application the mechanism of action of the compound to be genetically analyzed is at least partially understood.

Methods for performing clinical trials are well known in the art. (see e.g. Guide to Clinical Trials by Bert Spilker, Raven Press, 1991; The Randomized Clinical Trial and Therapeutic Decisions by Niels Tygstrup (Editor), Marcel Dekker; Recent Advances in Clinical Trial Design and Analysis (Cancer Treatment and Research, Ctar 75) by Peter F. Thall (Editor) Kluwer Academic Pub, 1995. Clinical Trials: A Methodologic Perspective by Steven Piantadosi, Wiley Series in Probability and Statistics, 1997). However, performing a clinical trial to test the genetic contribution to interpatient variation in drug response entails additional design considerations, including (i) defining the genetic hypothesis or hypotheses, (ii) devising an analytical strategy for testing the hypothesis, including determination of how many patients will need to be enrolled to have adequate statistical power to measure an effect of a specified magnitude (power analysis), (iii) definition of any primary or secondary genetic endpoints, and (iv) definition of methods of statistical genetic analysis, as well as other aspects. In the outline below some of the major types of genetic hypothesis testing, power analysis and statistical testing and their application in different stages of the drug development process are reviewed. One skilled in the art will recognize that certain of the methods will be best suited to specific clinical situations, and that additional methods are known and can be used in particular instances.

A. Performing a Clinical Trial: Overview

As used herein, a “clinical trial” is the testing of a therapeutic intervention in a volunteer human population for the purpose of determining whether it is safe and/or efficacious in the treatment of a disease, disorder, or condition. The present invention describes methods for achieving superior efficacy and/or safety in a genetically defined subgroup defined by the presence or absence of at least one gene sequence variance, compared to the effect that could be obtained in a conventional trial (without genetic stratification).

A “clinical study” is that part of a clinical trial that involves determination of the effect of a candidate therapeutic intervention on human subjects. It includes clinical evaluation of physiologic responses including pharmacokinetic (bioavailability as affected by drug absorption, distribution, metabolism and excretion) and pharmacodynamic (physiologic response and efficacy) parameters. A pharmacogenetic clinical study (or clinical trial) is a clinical study that involves testing of one or more specific hypotheses regarding the interaction of a genetic variance or variances (or set of variances, i.e. haplotype or haplotypes) on response to a therapeutic intervention. Pharmacogenetic hypotheses are formulated before the study, and may be articulated in the study protocol in the form of primary or secondary endpoints. For example an endpoint may be that in a particular genetic subgroup the rate of objectively defined responses exceeds the response rate in a control group (either the entire control group or the subgroup of controls with the same genetic signature as the treatment subgroup they are being compared to) or exceeds that in the whole treatment group (i.e. without genetic stratification) by some predefined relative or absolute amount.

For a clinical study to commence enrollment and proceed to treat subjects at an institution that receives any federal support (most medical institutions in the U.S.), an application that describes in detail the scientific premise for the therapeutic intervention and the procedures involved in the study, including the endpoints and analytical methods to be used in evaluating the data, must be reviewed and accepted by a review panel, often termed an Institutional Review Board (IRB). Similarly any clinical study that will ultimately be evaluated by the FDA as part of a new drug or product application (or other application as described below), must be reviewed and approved by an IRB. The IRB is responsible for determining that the trial protocol is safe, conforms to established ethical principles and guidelines, has risks proportional to any expected benefits, assures equitable selection of patients, provides sufficient information to patients (via a consent form) to insure that they can make an informed decision about participation, and insures the privacy of participants and the confidentiality of any data collected. (See the report of the National Commission for Protection of Human Subjects of Biomedical and Behavioral Research (1978). The Belmont Report: Ethical Principles and Guidelines for the Protection of Human Subjects of Research. Washington, D.C.: DHEW Publication Number (OS) 78-0012. For a recent review see: Coughlin, S. S. (ed.) (1995) Ethics in Epidemiology and Clinical Research. Epidemiology Resources, Newton, Mass.) The European counterpart of the U.S. FDA is the European Medicines Evaluation Agency (EMEA). Similar agencies exist in other countries and are responsible for insuring, via the regulatory process, that clinical trials conform to similar standards as are required in the U.S. The documents reviewed by an IRB include a clinical protocol containing the information described above, and a consent form.

It is also customary, but not required, to prepare an investigator's brochure which describes the scientific hypothesis for the proposed therapeutic intervention, the preclinical data, and the clinical protocol. The brochure is made available to any physician participating in the proposed or ongoing trial.

The supporting preclinical data is a report of all the in vitro, in vivo animal or previous human trial or other data that supports the safety and/or efficacy of a given therapeutic intervention. In a pharmacogenetic clinical trial the preclinical data may also include a description of the effect of a specific genetic variance or variances on biochemical or physiologic experimental variables in vitro or in vivo, or on treatment outcomes, as determined by in vivo studies in animals or humans (for example in an earlier trial), or by retrospective genetic analysis of clinical trial or other medical data (see below) used to formulate or strengthen a pharmacogenetic hypothesis. For example, case reports of unusual pharmacological responses in individuals with rare alleles (e.g. mutant alleles), or the observation of clustering of pharmacological responses in family members may provide the rationale for a pharmacogenetic clinical trial.

The clinical protocol provides the relevant scientific and therapeutic introductory information, describes the inclusion and exclusion criteria for human subject enrollment, including genetic criteria if relevant (e.g. if genotype is to be among the enrollment criteria), describes in detail the exact procedure or procedures for treatment using the candidate therapeutic intervention, describes laboratory analyses to be performed during the study period, and further describes the risks (both known and possible) involving the use of the experimental candidate therapeutic intervention. In a clinical protocol for a pharmacogenetic clinical trial, the clinical protocol will further describe the genetic variance and/or variances hypothesized to account for differential responses in the normal human subjects or patients and supporting preclinical data, if any, a description of the methods for genotyping, genetic data collection and data handling as well as a description of the genetic statistical analysis to be performed to measure the interaction of the variance or variances with treatment response. Further, the clinical protocol for a pharmacogenetic clinical trial will include a description of the genetic study design. For example patients may be stratified by genotype and the response rates in the different groups compared, or patients may be segregated by response and the genotype frequencies in the different responder or nonresponder groups measured. One or more gene sequence variances or a combination of variances and/or haplotypes may be studied.

The informed consent document is a description of the therapeutic intervention and the clinical protocol in simple language (e.g. third grade level) for the patient to read, understand, and, if willing, agree to participate in the study by signing the document. In a pharmacogenetic clinical study the informed consent document will describe, in simple language, the use of a genetic test or a limited set of genetic tests to determine the subject or patient's genotype at a particular gene variance or variances, and to further ascertain whether, in the study population, particular variances are associated with particular clinical or physiological responses. The consent form should also describe procedures for assuring privacy and confidentiality of genetic information.

The U.S. FDA reviews proposed clinical trials through the process of an Investigational New Drug Application (IND). The IND is composed of the investigator's brochure, the supporting in vitro and in vivo animal or previous human data, the clinical protocol, and the informed consent forms. In each of the sections of the IND, a specific description of a single allelic variance or a number of variances to be tested in the clinical study will be included. For example, in the investigator's brochure a description of the gene or genes hypothesized to account, at least in part, for differential responses will be included as well as a description of a genetic variance or variances in one or more candidate genes. Further, the preclinical data may include a description of in vivo, in vitro or in silico studies of the biochemical or physiologic effects of a variance or variances (e.g., haplotype) in a candidate gene or genes, as well as the predicted effects of the variance or variances on efficacy or toxicology of the candidate therapeutic intervention. The results of retrospective genetic analysis of response data in patients treated with the candidate therapy may be the basis for formulating the genetic hypotheses to be tested in the prospective trial. The U.S. FDA reviews applications with particular attention to safety and toxicological data to ascertain whether candidate compounds should be tested in humans.

The established phases of clinical development are Phase I, II, III, and IV. The fundamental objectives for each phase become increasingly complex as the stages of clinical development progress. In Phase I, safety in humans is the primary focus. In these studies, dose-ranging designs establish whether the candidate therapeutic intervention is safe in the suspected therapeutic concentration range. However, it is common practice to obtain information about surrogate markers of efficacy even in phase I clinical trials. In a pharmacogenetic clinical trial there may be an analysis of the effect of a variance or variances on Phase I safety or surrogate efficacy parameters. At the same time, evaluation of pharmacokinetic parameters (e.g., adsorption, distribution, metabolism, and excretion) may be a secondary objective; again, in a pharmacogenetic clinical study there may be an analysis of the effect of sequence variation in genes that affect absorption, distribution, metabolism and excretion of the candidate compound on pharmacokinetic parameters such as peak blood levels, half life or tissue distribution of the compound. As clinical development stages progress, trial objectives focus on the appropriate dose and method of administration required to elicit a clinically relevant therapeutic response. In a pharmacogenetic clinical trial, there may be a comparison of the effectiveness of several doses of a compound in patients with different genotypes, in order to identify interactions between genotype and optimal dose. For this purpose the doses selected for late stage clinical testing may be greater, equal or less than those chosen based upon preclinical safety and efficacy determinations. Data on the function of different alleles of genes affecting pharmacokinetic parameters could provide the basis for selecting an optimal dose or range or doses of a compound or biological. Genes involved in drug metabolism may be particularly useful to study in relation to understanding interpatient variation in optimal dose. Genes involved in drug metabolism include the cytochrome P450s, especially 2D6, 3A4, 2C9, 2E1, 2A6 and 1A1; the glucuronyltransferases; the acetyltransferases; the methyltransferases; the sulfotransferases; the glutathione system; the flavine monooxygenases and other enzymes known in the art.

An additional objective in the latter stages of clinical development is demonstration of the effect of the therapeutic intervention on a broad population. In phase III trials, the number of individuals enrolled is dictated by a power analysis. The number of patients required for a given pharmacogenetic clinical trial will be determined by prior knowledge of variance or haplotype frequency in the study population, likely response rate in the treated population, expected magnitude of pharmacogenetic effect (for example, the ratio of response rates between a genetic subgroup and the unfractionated population, or between two different genetic subgroups); nature of the genetic effect, if known (e.g. dominant effect, codominant effect, recessive effect); and number of genetic hypotheses to be evaluated (including number of genes and/or variances to be studied, number of gene or variance interactions to be studied). Other considerations will likely arise in the design of specific trials.

Clinical trials should be designed to blind both human subjects and study coordinators from biasing that may otherwise occur during the testing of a candidate therapeutic invention. Often the candidate therapeutic intervention is compared to best medical treatment, or a placebo (a compound, agent, device, or procedure that appears identical to the candidate therapeutic intervention but is therapeutically inert). The combination of a placebo group and blind controls for potentially confounding factors such as prejudice on the part of study participants or investigators, insures that real, rather than perceived or expected, effects of the candidate therapeutic intervention are measured in the trials. Ideally blinding extends not only to trial subjects and investigators but also to data review committees, ancillary personnel, statisticians, and clinical trial monitors.

In pharmacogenetic clinical studies, a placebo arm or best medical control group may be required in order to ascertain the effect of the allelic variance or variances on the efficacy or toxicology of the candidate therapeutic intervention as well as placebo or best medical therapy. It will be important to assure that the composition of the control and test populations are matched, to the degree possible, with respect to genetic background and allele frequencies. This is particularly true if the variances being investigated may have an effect on disease manifestations (in addition to a hypothesized effect on response to treatment). It is likely that standard clinical trial procedures such as insuring that treatment and control groups are balanced for race, sex and age composition and other non-genetic factors relevant to disease will be sufficient to assure that genetic background is controlled, however a preferred practice is to explicitly test for genetic stratification between test and control groups. Methods for minimizing the possibility of spurious results attributable to genetic stratification between two comparison groups include the use of surrogate markers of geographic, racial and/or ethnic background, such as have been described by Rannala and coworkers. (See, for example: Rannala B, and J L Mountain. 1997 Detecting immigration by using multilocus genotypes. Proc Natl Acad Sci USA Aug 19;94(17):9197-201.) One procedure would be to assure that surrogate markers of genetic background (such as those described by Rannala and Mountain) occur at comparable frequency in two comparison groups.

Open label trials are unblinded; in single blind trials patients are kept unaware of treatment assignments; in double blind trials both patients and investigators are unaware of the treatment groups; a combination of these procedures may be instituted during the trial period. Pharmacogenetic clinical trial design may include one or a combination of open label, single blind, or double blind clinical trial designs. Reduction of biases attributable to the knowledge of either the type of treatment or the genotype of the normal subjects or patients is an important aspect of study design. So, for example, even in a study that is single blind with respect to treatment, it should be possible to keep both patients and caregivers blinded to genotype during the study.

In designing a clinical trial it is important to include termination endpoints such as adverse clinical events, inadequate study participation either in the form of lack of adherence to the clinical protocol or loss to follow up, (e.g. such that adequate power is no longer assured), lack of adherence on the part of trial investigators to the trial protocol, or lack of efficacy or positive response within the test group. In a pharmacogenetic clinical trial these considerations obtain not only in the entire treatment group, but also in the genetically defined subgroups. That is, if a dangerous toxic effect manifests itself predominantly or exclusively in a genetically defined subpopulation of the total treatment population it may be deemed inappropriate to continue treating that genetically defined subgroup. Such decisions are typically made by a data safety monitoring committee, a group of experts not including the investigators, and generally not blinded to the analysis, who review the data from an ongoing trial on a regular basis.

It is important to note that medicine is a conservative field, and clinicians are unlikely to change their behavior on the basis of a single clinical trial. Thus it is likely that, in most instances, two or more clinical trials will be required to convince physicians that they should change their prescribing habits in view of genetic information. Large scale trials represent one approach to providing increased data supporting the utility of a genetic stratification. In such trials the stringent clinical and laboratory data collection characteristic of traditional trials is often relaxed in exchange for a larger patient population. Important goals in large scale pharmacogenetic trials will include establishing whether a pharmacogenetic effect is detectable in all segments of a population. For example, in the North American population one might seek to demonstrate a pharmacogenetic effect in people of African, Asian, European and Hispanic (i.e. Mexican and Puerto Rican) origin, as well as in native American people. (It generally will not be practical to segment patients by geographical origin in a standard clinical trial, due to loss of power.) Another goal of a large scale clinical trial may be to measure more precisely, and with greater confidence, the magnitude of a pharmacogenetic effect first identified in a smaller trial. Yet another undertaking in a large scale clinical trial may be to examine the interaction of an established pharmacogenetic variable (e.g. a variance in gene A, shown to affect treatment response in a smaller trial) with other genetic variances (either in gene A or in other candidate genes). A large scale trial provides the statistical power necessary to test such interactions.

In designing all of the above stages of clinical testing investigators must be attentive to the statistical problems raised by testing multiple different hypotheses, including multiple genetic hypotheses, in subsets of patients. Bonferroni's correction or other suitable statistical methods for taking account of multiple hypothesis testing will need to be judiciously applied. However, in the early stages of clinical testing, when the main goal is to reduce the large number of potential hypotheses that could be tested to a few that will be tested, based on limited data, it may be impractical to rigidly apply the multiple testing correction.

B. Phase I Clinical Trials

1. Introduction

Phase I clinical trials are generally designed primarily to establish a safe dose and schedule of administration for a new compound. At the same time, Phase I is the first opportunity to study the clinical pharmacology of a new compound in man. Relevant studies may include aspects of pharmacokinetic behavior, side effects and toxicity. In addition to these well established purposes, Phase I trials are increasingly being used to gather information relevant to early assessment of efficacy. Such information can be useful in making an early yes/no decision about the further development of a compound, or a family of related compounds, all being tested simultaneously in Phase I trials. Since Phase I trials are typically conducted in normal volunteers (compounds for cancer and some other terminal diseases are an exception), surrogate markers of drug effect are measured, rather than disease response. The development of sophisticated surrogate markers of pharmacodynamic effects has allowed more information on efficacy to be gathered in Phase I, and this trend will almost certainly continue as basic understanding of disease pathophysiology increases, and as more products are developed for disease prophylaxis.

Phase I studies are typically performed on a small number (<60) of healthy volunteers. Consequently, Phase I studies as currently designed are not amenable to genetic analysis: the number of subjects is simply too small to detect, with adequate statistical certainty, any genetic effects on drug response that are short of all or none in magnitude. In fact, no genetic analyses of Phase I studies have been published or described in public meetings.

As described in detail elsewhere in this application, it is highly desirable to gather the information necessary to make informed decisions about clinical development as early as possible in the development process, particularly once human testing has begun and costs therefore mount quickly. Timely information may allow a drug to be killed early, or may result in an accelerated program of clinical trials. In addition to information about efficacy and safety, it is useful to have information about the existence and magnitude of genetic effects on efficacy and toxicity at the earliest possible stage. If properly managed, genetically determined heterogeneity in drug response may not be an obstacle to development. On the contrary, it may provide the basis for identification of a patient population in whom both high efficacy and safety can be achieved. Clear delineation of such a population may facilitate smaller, more targeted trials and more rapid clinical development. Consequently, the early identification of genetic determinants of drug response will, in the future, increasingly become a priority of clinical development.

Phase I trials are not necessarily confined to the initial stages of human clinical development. It is not unusual for Phase I trials to be initiated at a later stage of clinical development in order to, for example, clarify basic questions about clinical pharmacology that have arisen as a result of Phase II study data. It may be that the most efficient way to advance the genetic understanding of pharmacological responses to a compound in Phase II is to perform a Phase I trial using a specific genetic design, as described below.

2. Phase I Trials Designed for Genetic Analysis

In this invention we describe two exemplary novel methods for organization of Phase I trials that will facilitate identification and measurement of the genetic component of variation in treatment response using modest numbers of subjects. We describe how these methods can be practiced by selectively enrolling subjects who share genetic characteristics, either as a result of a familial relationship or as a result of genetic homogeneity at candidate loci believed to affect response to the candidate treatment. We show how the analysis of such individuals substantially increases the power of genetic analysis compared to analysis of unrelated individuals. We also describe methods for operating a Phase I unit capable of carrying out the novel genetic analyses

The two types of Pharmacogenetic Phase I Units described in this application will be referred to as the Pharmacogenetic Phase I Relatives Unit and the Pharmacogenetic Phase I Outliers Unit, or the Relatives Unit and the Outliers Unit for short. The term Pharmacogenetic Phase I Unit will be used to refer to both types of Phase I Unit. The Relatives Unit requires a population comprised of groups of related individuals. The related individuals may be parents and offspring, groups of sibs, or of cousins, or any mixture of these or other groups of related individuals. The Outliers Unit requires the initial enrollment of a large number of unrelated volunteers (at least several hundreds of subjects, preferably at least one thousand, more preferably at least five thousand, and most preferably ten thousand or more individuals) willing to provide DNA for genotyping on an as-needed basis (many of these volunteers will never participate in a trial). Subsequently, small numbers of individuals are drawn from this large population for specific clinical trials, based on their genetic homogeneity at candidate loci believed likely to account for intersubject variation in response to the candidate compound.

The concept underlying these two types of Pharmacogenetic Phase I Units is similar: the idea is to recruit multiple small groups of subjects who are genetically more homogeneous than would be possible with standard nongenetic recruitment criteria. If there is a genetic component to treatment response then there should be more intragroup homogeneity and more intergroup heterogeneity in drug response measures (e.g. surrogate measures of drug response) than would be expected by chance, and there should be statistically significant differences in drug response measures between the different groups. The magnitude of such differences can provide an estimate of the magnitude of the genetic component of intersubject variation in drug response.

3. Pharmacogenetic Phase I Relatives Unit

In the Pharmacogenetic Phase I Relatives Unit, one is comparing groups of related individuals to each other and to other groups of related individuals. The underlying assumption is that one can assess the magnitude of the genetic component of variation in drug response (if any) by comparing drug response traits in related individuals with those of unrelated individuals. Two types of effect would suggest the presence of a genetic component to variation in drug response measures. First, the distribution of drug responses in related individuals may be different from that observed in the entire group, or in a group comprised of unrelated individuals. For example, a statistically significant narrowing of the distribution (e.g. smaller standard deviation in groups of related individuals compared to unrelated individuals) would indicate that individuals who share alleles are more similar to each other than individuals who do not share (as many) alleles, implying that the drug response trait is partially affected by a heritable factor or factors. Second, the mean value of the drug response measure (whether blood pressure or a cognitive test) may vary between groups of related individuals, indicating that different alleles at loci relevant to drug response are present in the different families. (Note that the relevant trait is not blood pressure or cognition, but the response of blood pressure or cognition to a pharmacological intervention.)

Individuals can be related in any of several ways, most preferably as parent and child or as siblings. Parent-child pairs, in particular, enable one to use simple statistical techniques (e.g., regression) in order to assess the degree to which response to surrogate markers is influenced by genetic differences among individuals. However, parent-child pairs may be less suitable for some surrogate markers, especially those related to candidate drugs used to treat age-related disorders. In such a context, one can readily use clusters of siblings and/or cousins, uncle/nephew pairs or other groups of related individuals to assess the degree of genetic determination of response to a surrogate marker.

An attractive aspect of the Pharmacogenetic Phase I Relatives Unit (unlike the Outliers Unit) is that it does not require any laboratory tests to implement. One infers the degree of gene sharing between individuals from their relationship to each other. A parent is 50% genetically identical to each of his or her children; sibs are 50% genetically identical to each other on average; uncles/aunts are 25% identical to nieces/nephews on average, and so forth. Thus the degree to which two related individuals are expected to be similar as a result of genetic factors is known. Therefore no tests to determine genetic status are required (i.e. no genotyping); in fact, no knowledge of the relevant candidate loci is required at all (albeit knowledge of the relevant genes is required to develop a useful genetic diagnostic test at a later stage). Thus, the Relatives Unit provides a clear picture of the importance of heredity factors in determining drug response, regardless of our understanding of the mechanism of action of the drug, or any other aspect of drug pharmacology.

The rationale is as follows: if a surrogate drug response trait (i.e., a surrogate marker of pharmacodynamic effect that can be measured in normal subjects) is under genetic control, then related individuals, such as sibs (who share 50% of their alleles at autosomal loci on average), should have more similar responses than unrelated individuals, who share a much smaller fraction of alleles. In other words, individuals who share more alleles at the loci that affect drug response should be more similar to each other than individuals who, on average, share fewer alleles. By using statistical methods known in the art the distribution of traits of related individuals can be compared to the degree of variation in a set of unrelated individuals. The potential for insight from this kind of analysis is reflected in the fact that twin studies (in which traits of identical twins are compared to those of fraternal twins) indicate that differences among individuals in pharmacokinetic variables (e.g. compound half life, peak concentration) can be strongly genetically determined. (For a summary of such pharmacokinetic studies, see Propping, P. [1978] Pharmacogenetics. Rev. Physiol. Biochem. Pharmacol. 83: 123-173.) Such studies are important because they clearly reveal genetic determination of pharmacogenetic traits (although they may overestimate its degree; see Falconer, D. S. and Mackay, T. [1996] Introduction to Quantitative Genetics, Addison Wesley Longman Ltd.).

The type of study proposed here, whether it involves comparison of parents and offspring, groups of sibs, or other groups of relatives, will also reveal the extent of genetic determination, and without requiring twins. This is a two-fold advantage; pairs of twins are more difficult to obtain than parent-child or sib-sib pairs, and one avoids the uncertainty about the genetic inferences gained from twin analysis.

Drug responses among related and unrelated individuals may be continuously or discretely distributed. In the former case, it is likely that many loci have some effect on the trait, while in the latter case, variation could be attributable to Mendelian segregation of alleles in a family (or families) with, for example, AA homozygotes giving one phenotype and Aa heterozygotes and aa homozygotes giving a second phenotype, all in the context of a relatively homogeneous genetic background.

There is a wealth of analytical techniques known in the art that can be used to assess the mode of inheritance for a particular trait and to determine the degree to which differences among individuals are genetically determined. These techniques include cluster analysis and discriminant analysis used to define traits with variable expression and the fitting of a variety of genetic models to the data, including generalized single-locus models, mixed models in which a trait is determined by a major locus and by many minor loci, and a so-called polygenic model in which many loci contribute variation to the trait, the result being a continuously-distributed phenotype (For further details, see Eaves, L. J. [1977] Inferring the causes of human variation, Journal of the Royal Statistical Society A 140: 324-355 and Cloninger, C. R. [1988] Complex Human Traits. Pp. 312-317 in: Proceedings of the Second International Conference on Quantitative Genetics, eds., B. S. Weir, E. J. Eisen, M. M. Goodman, and G. Namkoong, Sinauer Associates, Inc). Specific statistical techniques involved in the fitting and analysis of these genetic models are also well known in the art; they include parametric and nonparametric correlation, regression, and one-way and two-way analysis of variance (For further details, see Mather, K. and Jinks, J. L. [1977] Introduction to Biometrical Genetics, Cornell University Press and Falconer, D. S. and Mackay, T. [1996] Introduction to Quantitative Genetics, Addison Wesley Longman Ltd.) Many, perhaps most, traits of pharmacogenetic interest will be continuously-distributed. In this context, the central statistical comparison is one between the differences among average traits of different families (say, groups of sibs), or among all the members of several such families, as compared to the differences among traits within families (among sibs). If such differences in so-called mean squares are large enough (as compared to the differences expected under the null hypothesis of no family differences), one can infer that there is a genetic component to differences among families.

Standard theory known in the art indicates that there is an inverse relationship between study size and the ability to detect a given genetic effect. So, for example, assume that the 50% of the variation among individuals is due to genetic differences. A Phase 1 trial composed of sixty individuals consisting of thirty parent-child pairs may or may not allow one to detect such a genetic effect, given the standard criterion for statistical significance (P<0.05), depending on assumptions one makes about the number of loci that have major effects. However, a trial composed of 120 individuals consisting of sixty parent-child pairs would likely be sufficient to provide statistically significant evidence for a 50% heritable drug response effect. Once one parent-child pair is recruited, it is generally advantageous statistically to add additional parent-child combinations as opposed to adding additional children for a given parent.

If 75% or more of the variation in drug response among individuals is due to genetic differences, a Phase 1 trial composed of sixty individuals consisting of thirty parent-child pairs would allow one to detect such a genetic effect, given the standard criterion for statistical significance (P<0.05).

Similar calculations can be made if one analyzes siblings in a Phase I trial, instead of using parent-child pairs. These calculations indicate that the more powerful approach for a Relatives Unit is generally to focus on parent-child pairs as opposed to the use of groups of siblings, especially if minimizing the number of subjects is an objective of the study. However, the use of groups of siblings may be necessary or preferable, especially if the trait in question is manifested only at a specific age. In such a case, one can readily use standard theory to compare alternative designs for the study. The overall point is that the statistical framework associated with the Relatives Unit will allow one to choose the approach that is best-suited for a given trait.

In general, techniques for measuring whether pharmacodynamic traits are under genetic control using surrogate markers of drug efficacy will be useful in obtaining an early assessment of the extent of genetically determined variation in drug response for a given therapeutic compound. Such information provides an informed basis for either stopping development at the earliest possible stage or, preferably, continuing development, but with a plan to identify and control for genetic variation so as to allow rapid progression through the regulatory approval process.

For example, it is well known that clinical trials to assess the efficacy of candidate drugs for Alzheimer's disease are long and expensive, and most such drugs are only effective in a fraction of patients. Using surrogate measures of response in normals drawn from a population of related individuals might help to assess the contribution of genetic variation to variation in treatment response. For an acetylcholinesterase inhibitor, relevant surrogate pharmacodynamic measures might include testing erythrocyte membrane acetylcholinesterase levels in drug treated normal subjects, or testing performance on a psychometric test of short term memory, or other measures that are affected by treatment (and ideally that correlate with clinical efficacy).

Similarly, antidepressant drugs can produce a variety of effects on mood in normal subjects. Careful measurement and statistical analysis of such responses in related and unrelated normal subjects could provide an early indication of whether there is a genetic component to drug response (and hence clinical efficacy). The observation of significant variation among families would provide evidence of a pharmacogenetic effect and justify the substantial expenditure necessary for a full pharmacogenetic drug development program. Conversely, the absence of any significant familial influence on drug response in a Pharmacogenetics Relatives Unit could provide an early termination point for pharmacogenetic studies.

Again, the proposed studies do not require any knowledge of candidate loci, nor is DNA collection or genotyping required. One needs only a reliable surrogate pharmacodynamic assay and groups of related normal individuals. Standard statistical methods should permit the magnitude of the pharmacogenetic effect to be estimated. It should be a criteria for deciding whether to proceed with more intensive, gene-focused pharmacogenetic analysis during later stages of development.

4. Pharmacogenetic Phase I Outliers Unit

The prerequisites for a Pharmacogenetic Phase I Outliers Unit, as well as the type of information that can be obtained, differ in several respects from a Pharmacogenetic Phase I Relatives Unit. First, the Outliers Unit requires some knowledge of the molecular pharmacology of the candidate compound—enough knowledge to select at least one candidate gene. Second, the Outliers Unit provides information on the effect, if any, of known genetic variation in the candidate gene or genes on variation in the drug response measures. This is advantageous in that it sets the stage for pharmacogenetic analysis in later stages of clinical development. Third, the Outliers Unit does not require recruitment of relatives. Instead, one initially recruits a large population of individuals from which small subsets are drawn as necessary for specific trials based on their genotypes. All of the individuals in the large population are initially asked to provide DNA samples (from blood or other readily available tissue such as buccal mucosa) which can subsequently be genotyped at candidate loci of potential relevance to a particular candidate drug of interest. Over time a database of genotypes can be assembled, potentially reducing the need for genotyping later. From this large collection of subjects one then selects a group of individuals with genotypes expected to homogeneous for the drug response trait of interest (assuming that the candidate gene(s) play a significant role in drug response). The individuals with identical (and preferably homozygous) genotypes at the candidate gene(s) might comprise a collection of the common genotypes or haplotypes, or they may include some rare genotypes/haplotypes as well. The main point is that one can recruit groups consisting of any mixture of genotypes or haplotypes in order to assess the role that variation in the candidate gene(s) may play in trait determination. In this method, then, one recruits a population for clinical genetic investigation utilizing methods in statistical genetics to optimize the size and genetic composition of the population.

The mechanics of an Outlier Unit are as follows. Several thousand subjects are enrolled in the Outlier Unit with the understanding that they provide a blood sample from which DNA is extracted and stored. Each time a new outlier study is performed their sample may be genotyped. (It will not be necessary to genotype all subjects for all trials—just enough to identify subjects with the desired genotypes or haplotypes. Subjects may be paid a fee for each genotyping analysis done on their sample, regardless of whether the sample is used.) Only rarely will a particular subject have a genotype that meets the criteria for a specific outlier study (see below). When a match occurs, that subject will be invited to participate in that study. The genotyping done to identify subjects for a study will be determined by the candidate genes deemed relevant to pharmacology of the candidate drug, and by the polymorphisms or haplotypes in those candidate genes. Ideally DNA samples from several thousand subjects will be arrayed in 96 or 384 well plates so that the genotyping or haplotyping of large numbers of subjects can be performed using automated methods. Any highly accurate and inexpensive genotyping procedure will suffice, such as the methods described elsewhere in this application. Clearly it is desirable to have a stable population for genotyping, given the investment required to recruit subjects, isolate and array DNA, and accumulate a database of genotype data. Since most subjects will only rarely be invited to participate in clinical trials, the ongoing participation of subjects in the Outliers Unit must be assured by other means—for example, by a modest annual payment for remaining in the Outliers Unit, plus a fee for each occasion on which their sample is genotyped.

The power of the Outliers Unit lies in the ability to rapidly enroll individuals with virtually any desired genotype in a Phase I clinical trial. Suppose, for example, that one wants to determine the drug response phenotype of individuals homozygous for rare alleles at candidate loci. Consider a compound for which there are two loci believed likely to influence response to treatment. The first locus has alleles A and a, while the second has alleles B and b. If these loci do in fact contribute significantly to treatment response then homozygotes would be expected to exhibit the most extreme responses (assuming a dominant or codominant model). One could also measure epistatic (gene X gene) interactions on the presumption that drug response measures might be extreme in individuals homozygous for specific alleles of the two candidate genes. So, for example, one would perform a Phase I study consisting of measuring a surrogate drug response in individuals with genotypes AA/BB, aa/BB, AA/bb and aa/bb and then statistically comparing the distribution of a trait in each of these groups with the distribution of the same trait in the other groups and/or in the unfractionated (total) population. The statistical techniques for such comparisons are known in the art and include parametric and nonparametric analyses to detect differences in population averages, such as the t-test and the Mann-Whitney U test. If individuals of a given rare genotype do have significantly different surrogate drug responses when compared to each other, or when compared to the rest of the population, one can infer that the locus likely affects the trait.

The size requirements of the source population of individuals will depend on the range of allele frequencies to be analyzed. For example, if the allele frequencies for A and a are, say, 0.15 and 0.85, and for B and b are 0.2 and 0.8 then the frequency of AA homozygotes is expected to be 2.25% and BB homozygotes 4%. In the absence of any linkage between the loci, the frequency of AA/BB double homozygotes is expected to be 0.0225×0.04=0.0009 or about one subject in 1000. At least five subjects of each genotype should be recruited for the Outlier Unit, and preferably at least ten subjects. Thus, for studies of two loci in which the minor allele frequency for both loci is in the 0.15-0.20 range, the recruitment of individuals that are potential outliers for the trait under investigation (i.e., homozygotes at the candidate loci) will require at least 1,000 individuals and preferably 5,000 or more.

One of the most useful aspects of the Outlier Unit is that individuals with rare genotypes can be pharmacologically assessed in a small study. This addresses a serious limitation of conventional clinical trials with respect to the investigation of polygenic traits or the effect of rare alleles. Even conventional Phase III studies, which typically have the largest number of patients, are usually of insufficient size to address simple one-locus hypotheses about efficacy or toxicity with adequate statistical power (e.g. 80% or 90% power). The problem is that for each new allele that must be considered (e.g. five common haplotypes at a candidate locus) the comparison groups are reduced and statistical power is diminished. It is therefore an especially challenging problem to test the effect of multiple alleles at a single locus, let alone interaction of alleles at several loci in determining drug response. The Outlier Unit provides a way to efficiently test for the effects of multiple alleles at a candidate locus (e.g. haplotypes), or to test for interactions between two or more candidate loci by allowing ready identification of groups of individuals who, on account of being homozygous at one or several loci of interest, should be outliers for the drug response traits of interest.

The information that can be gained from an Outliers Unit is of great value in designing subsequent efficacy trials, as it provides a basis for constraining the number of hypotheses to be tested. In lieu of such information, one is compelled to statistically test a variety of genetic models for a number of candidate loci. The correction for multiple testing necessitated by such uncertainty about the genetic model is frequently large enough to put statistically significant results beyond reach. On the other hand, if the phenotypic effect of each allele at a locus (or the effect of at least some alleles) is known from the Outliers Unit study, one is then able to design a Phase II or Phase III study that tests a relatively small number of genetic hypotheses, thereby considerably improving the statistical power of the genetic analysis in efficacy trials.

Consider a locus with two alleles, one with frequency 0.95 and the other 0.05, as revealed by genotyping the individuals in the large source population for the Outliers Unit. The two alleles combine to make three genotypes which are observed to differ in their response to a candidate compound of interest. There are several statistical comparisons that one can undertake in order to determine whether different alleles at this locus are associated with differences in response. One is to compare the average response of, say, individuals who are homozygous for the rare allele with the average response of individuals chosen at random from the source population. In this instance, the Outlier Unit is composed of a group of individuals with the rare genotype and an equal-sized group composed of random genotypes (including the rare genotype). (In general, equal group sizes are statistically more efficient; they are not necessary, however, which is fortunate since some alleles of interest might be so rare that finding, say, even ten individuals who are homozygous would be difficult.) A second kind of statistical comparison would be to compare equal-sized groups of the three genotypes (AA, Aa, aa), in order to determine whether the presence or absence of a particular allele has a significant effect on the drug response trait. In this instance, the Outlier Unit is preferably composed of equal-sized groups of the three genotypes.

Assume that being a homozygote for the rare allele of the locus described in the preceding paragraph causes a 15% average difference in a pharmacokinetic parameter (e.g., the area under curve of drug concentration in blood) as compared to random individuals. Assume further that the Outliers Unit has a total of sixty individuals, including thirty individuals of the rare genotype and thirty individuals chosen at random. Finally, assume that the variance of individual responses is identical within the two groups and that it is equal to 0.1. Standard statistical theory indicates that thirty individuals per group is not adequate to statistically prove that there is a significant difference in average uptake rate between the groups (P<0.05). Instead, with an increase to 108 individuals in each group, one would be able to provide statistical evidence for this effect. However, if we assume that homozygosity for an allele at the candidate locus causes a 30% difference in area under curve then the number of individuals required to provide statistical evidence for a difference between the two groups (for P<0.05 and holding all other assumptions constant) is only twenty-seven. The number of individuals required to detect a 60% difference in area under curve (all other assumptions constant) is only seven. This calculation assumes that the loci in question affect only the average trait in each of the two groups and that the shapes of the trait distribution are identical in the two groups. While conclusions based upon such an assumption are biologically meaningful and statistically robust, in some circumstances there may be differences in the shape of the trait distributions associated with different genotypes. In particular, one or more classes of homozygous genotypes may have a narrower trait distribution (smaller variance) than another, or than the population as a whole. Such a difference can be accounted for in the analysis; in fact, it would be expected to reduce the number of subjects needed for the Outliers Unit trial (since the smaller variance of one distribution reduces the overlap between it and the other trait distributions to which it is being compared). In fact, the assumption of identical variances in the homozygote and total groups is not necessarily the biologically most likely case: it is reasonable to expect that the variance of the trait in the genetically more homogeneous group may be less (if the locus in question in fact contributes to variation in the drug response trait). This effect would result in a smaller population being adequate to show a genetically determined component to the difference in treatment effect between the two groups.

Serious adverse effects occuring at low frequency are often detected in the later stages of drug development. In some cases such effects have a significant genetic component. To address this issue preemptively, an Outlier Unit can perform trials in which subjects are selected to represent only the rare alleles at one or more loci that are candidates for influencing the response to treatment. For example, variances occurring at 5% allele frequency are expected to occur in homozygous form in 0.25% of the population (0.05×0.05), and therefore may rarely, if ever, be encountered in early clinical development. Yet such subjects could readily be identified by genotyping the hundreds to thousands of patients enrolled in a Phase I Outliers Unit.

Alternatively, by insuring that all common genotypes are represented in an Outlier Unit study the contribution of a major candidate locus can be tested with a powerful statistical design. Consider a locus with five haplotypes, A, B, C, D and E, with frequencies 0.3, 0.25, 0.2, 0.15, and 0.05 (plus several additional alleles with frequency lower than 0.05). A comparison of groups of homozygous for each of the haplotypes—that is AA, BB, CC, DD and EE homozygotes—each group of equal size, provides a powerful design to measure the contribution of variation at the candidate locus to variation in drug response In this case, determination of sample sizes rests upon assumptions about the differences in average trait values for each haplotype. All other things being equal, detecting a difference is easiest when a subset of the haplotypes appears to be appreciably distinct from the rest. Such a situation allows one to make a reasonably principled decision to lump haplotypes so that one compares, say, one haplotype with all of the others. In such a circumstance, sample size calculations for testing a difference in average responses would be roughly similar to those described above. More generally, one can assess the overall heterogeneity of the traits associated with each haplotype (say, with a parametric or nonparametric analysis of variance) and one can also make individual comparisons between haplotypes (by using a multiple comparison procedure if the initial analysis of variance reveals significant heterogeneity) The identification of genetically determined phenotypic variation at such a locus the can reduce the likelihood of discrepant results due to genetic stratification in later trials.

In another embodiment of the invention, it would be useful to prospectively determine the status of polymorphisms at genes that are involved in the pharmacokinetic or pharmacodynamic action of many drugs. This would save genotyping the large Outliers Unit population each time a new project is initiated. Demand for genotyped groups of patients can be anticipated from pharmaceutical and biotechnology companies and contract research organizations (CROs). Genotyping might initially focus on common pharmacological targets such as estrogen receptors or other nuclear receptors, or on adrenergic receptors, serotonin receptors, dopamine receptors and other G protein coupled receptors. The pre-genotyped Outlier Unit population could be part of a package of services (along with genotyping assay development capability, high-throughput genotyping capacity and software and expertise in statistical genetics) designed to accelerate pharmacogenetic Phase I studies. Eventually, as the databank of genotypes is expanded, individuals with virtually any genotype or combination of genotypes can be called in for precisely designed physiological or toxicological studies designed to test for pharmacogenetic effects.

As noted earlier, the Pharmacogenetic Phase I Relatives Unit and the Pharmacogenetic Phase I Outlier Unit can provide useful information at almost any stage of clinical development. It is not unusual, for example, for a product in Phase II or even Phase III testing to be remanded to Phase I in order to clarify some aspect of toxicology or physiology. In this context, either or both of the Pharmacogenetic Phase I Units would be extremely useful to a drug development company, as studies in groups of related individuals (Relatives Unit) or in defined genetic subgroups drawn from a large genotyped population (Outliers Unit) would be an economical and efficient way to clarify the nature and extent of pharmacogenetic effects, if any, thereby paving the way for future rational development of the compound.

5. Surrogate Endpoints

As explained above, some of the most attractive applications of Pharmacogenetic Phase I Units depend on the availability of surrogate markers for pharmacodynamic drug action. The most useful surrogate markers are those which can be used in normal subjects in Phase I; which can be measured easily, inexpensively and accurately, and for which there is compelling data linking the surrogate marker with some clinically important aspect of disease biology, such as disease manifestations in various organ systems, disease progression, disease morbidity or mortality, or disparate other clinical indices known in the art. The utility of surrogate markers increases in proportion to the difficulty and cost of clincal development. Thus for a disease like Alzheimer's, where long trials involving many patients are standard, the use of surrogate measures of, for example, cognitive ability, are highly desirable.

The standard endpoints of Phase I trials are also useful measures for analysis in a Pharmacogenetic Phase I Unit. For example, studies of compound adsorption, distribution, metabolism, excretion and bioavailability may be analyzed for their genetic component. Similarly, toxic responses and dose-related side effects may be analyzed by the pharmacogenetic methods of this invention.

6. Establishing and Operating a Phase I Pharmacogenetic Relatives Unit

First, it should be noted that the information that can be gained from a Pharmacogenetic Phase I Unit provides for substantial cost savings in later stages of clinical development. Therefore it is to be expected that even if the cost of operating a Pharmacogenetic Phase I Unit exceeds the cost of operating a conventional Phase I Unit, the overall costs of clinical development are likely to be lower, thereby justifying the costs of the Pharmacogenetic Phase I Unit. Nonetheless, it is clearly desirable to operate a Pharmacogenetic Phase I Unit as efficiently as possible. In order to make a Phase I unit an efficient business operation it is useful to (i) use statistical genetic methods to design studies that require the minimal number of subjects to achieve adequate statistical power (e.g. power of 80% to detect an effect at the P<0.05 level), in order to keep subject costs at a minimum, (ii) take measures to reduce the turnover of participating subjects, in view of the long term investment made in patient recruitment and (in the case of the Outliers Unit) genotyping. This may be accomplished by offering subjects financial or other incentives to encourage sustained participation in the Pharmacogenetic Phase I Unit. The types of incentives that would be useful differ between the two types of Phase I Units (see below). (iii) Secure rights to reuse genotype data and, ideally, phenotypic data collected during each Pharmacogenetic Phase I Unit trial, in order to create a database that over time will save costs by eliminating the need to repetitively genotype the same loci, and may eventually produce information of broad utility in clinical pharmacology research: namely a database on the heritability of phenotypic responses to various broad classes of compounds (benzodiazepines, statins, taxanes, etc.) and the major classes of genes involved. Such a database could become a product.

In order to efficiently set up a Phase I Pharmacogenetic Relatives Unit family participation can be encouraged by appropriate incentive compensation. For example, subjects with no participating family members might be paid $200 for participation in a study; two sibs participating in the same study might each be paid $300; if they could encourage another sib (or cousin) to participate the three related individuals might each be paid $350 for each study; parent-sib pairs might be paid $400 for each study, and so forth. This type of compensation would encourage subjects to recruit their relatives to participate in Phase I studies. To the extent that certain types of blood relationship are more useful for efficient genetical analysis, those types of related individuals could be compensated most highly. This type of compensation would increase the cost of studies, however the increased speed of setting up the Relatives Unit, and the increased retention of subjects, would compensate over time. The optimal location to establish a Pharmacogenetic Relatives Unit is in a city with a stable population, many large families, and a open attitudes toward modern technology. The size of a Relatives Unit need be little more than 150 subjects, though 250 would allow greater flexibility in drawing related subjects from different racial or ethnic groups (see below), and allow for more trials to be performed simultaneously. 400-500 subjects would be most preferable. Greater than 500 subjects would provide little benefit while increasing costs substantially.

Ideally subjects in the pharmacogenetic Phase I unit are of known ethnic/racial/geographic background and willing to participate in Phase I studies, for pay, over a period of years. For specific studies in a Relatives Unit subjects from one or more racial, ethnic or geographically defined group may be analyzed in order to (i) mirror the population in which Phase II or Phase III trials are to be conducted; (ii) determine if there are measurable differences in pharmacogenetic effects in different racial, ethnic or geographically defined groups; (iii) study the most homogeneous group possible in order to increase the chances of detecting a particular type of genetic effect.

Ideally consent for genotyping should be obtained at the same time that subjects are enrolled. Appropriate consent forms will be drafted and approved by an independent review board. It would be most efficient if blanket consent for genotyping any polymorphic site or sites deemed relevant to the pharmacology of any candidate drug could be obtained. However, if this somewhat broad type of consent is deemed inappropriate by the review board then consent could be somewhat narrowed by adding the qualification that any loci that are genotyped be relevant to a customer project. A third, more onerous arrangement would be obtain consent to genotype polymorphic sites in loci relevant to specific families of compounds, or to obtain consent for genotyping a specific list of genes. Another, still less desirable solution would be to obtain consent for genotyping on a project-by-project basis (for example by mailing out reply cards to all subjects for each study), after the specific polymorphic sites to be genotyped have been selected.

Another essential element of operating a Relatives Unit is having adequate quality control measures. One crucial aspect of quality control is an independent testing method to confirm the relatedness of the recruited subjects This can be accomplished by genotyping multiple (10-50) highly polymorphic loci, such as short tandem repeat sequences, in individuals believed to be related. By comparing the degree of genetic identity observed with that expected from the purported relation (e.g. 50% in the case of sibs) it is possible to ensure with considerable certainty that all related individuals are in fact related as they believe themselves to be. (Inconsistency between genotyping and reported relationship would be dealt with simply by not enrolling the unrelated individuals in any trials.)

As indicated above, methods for retention of subjects in a Phase I Outliers Unit preferably consist of making modest payments for continuing participation (i.e. continued permission to genotype under the limits of the consent); additional payments for genotyping analysis, whether or not it results in a request to participate in a clinical study; and, of course, generous compensation for participation in each Outliers Unit clinical study.

Phase I of clinical development is generally focused on safety, although drug companies are increasingly obtaining information on pharmacokinetics and surrogate pharmacodynamic markers in early trials. Phase I studies are typically performed with a small number (<60) of normal, healthy volunteers usually at single institutions. The primary endpoints in these studies usually relate to pharmacokinetic parameters (i.e. adsorption, distribution, metabolism and bioavailability), and dose-related side effects. In a Phase I pharmacogenetic clinical trial, stratification based upon allelic variance or variances of a candidate gene or genes related to pharmacokinetic parameters may allow early assessment of potential genetic interactions with treatment.

Phase I studies of some diseases (e.g. cancer or other medically intractable diseases for which no effective medical alternative exists) may include patients who satisfy specified inclusion criteria. These safety/limited-efficacy studies can be conducted at multiple institutions to ensure rapid enrollment of patients. In a pharmacogenetic Phase I study that includes patients, or a mixture of patients and normals, the status of a variance or variances suspected to affect the efficacy of the candidate therapeutic intervention may be used as part of the inclusion criteria. Alternatively, analysis of variances or haplotypes in patients with different treatment responses may be among the endpoints. It is not unusual for such a Phase I study design to include a double-blind, balanced, random-order, crossover sequence (separated by washout periods), with multiple doses on separate occasions and both pharmacokinetic and pharmacodynamic endpoints.

2. Phase I Trials with Subjects Drawn from Large Populations and/or from Related Volunteer Subjects: The Pharmacogenetic Phase I Unit Concept

In general it is useful to be able to assess the contribution of genetic variation to treatment response at the earliest possible stage of clinical development. Such an assessment, if accurate, will allow efficient prioritization of candidate compounds for subsequent detailed pharmacogenetic studies; only those treatments where there is early evidence of a significant interaction of genetic variation with treatment response would be advanced to pharmacogenetic studies in later stages of development. In this invention we describe methods for achieving early insight—in Phase I—into the contribution of genetic variation to variation in surrogate treatment response variables. It occurred to the inventors that this can be accomplished by bringing the power of genetic linkage analysis and outlier analysis to Phase I testing via the recruitment of a very large Phase I population including a large number of individuals who have consented in advance to genetic studies (occasionally referred to hereinafter as a Pharmacogenetic Phase I Unit). In one embodiment of a Pharmacogenetic Phase I Unit many of the subjects are related to each other by blood. (Currently Phase I trials are performed in unrelated individuals, and there is no consideration of genetic recruitment criteria, or of genetic analysis of surrogate markers.) There are several novel ways in which a large population, or a population comprised at least in part of related individuals, could be useful in early clinical trials. Some of the most attractive applications depend on the availability of surrogate markers for pharmacodynamic drug action which can be used early in clinical development, preferably in normal subjects in Phase I. Such surrogate markers are increasingly used in Phase I, as drug development companies seek to make early yes/no decisions about compounds.

Recruitment of a population optimized for clinical genetic investigation may entail utilization of methods in statistical genetics to select the size and composition of the population. For example powerful methods for detecting and mapping quantitative trait loci in sibpairs have been developed. These methods can provide some estimate of the statistical power derived from a given number of groups of closely related individuals. Ideally subjects in the pharmacogenetic Phase I unit are of known ethnic/racial/geographic background and willing to participate in Phase I studies, for pay, over a period of years. The population is preferably selected to achieve a specified degree of statistical power for genetic association studies, or is selected in order to be able to reliably identify a certain number of individuals with rare genotypes, as discussed below. Family participation could be encouraged by appropriate incentive compensation. For example, individual subjects might be paid $200 for participation in a study; two sibs participating in the same study might each be paid $300; if they could encourage another sib (or cousin) to participate the three related individuals might each be paid $350, and so forth. This type of compensation would encourage subjects to recruit their relatives to participate in Phase I studies. (It would also increase the cost of studies, however the type of data that can be obtained can not be duplicated with conventional approaches.) The optimal location to establish such a Phase I unit is a city with a stable population, many large families, and a positive attitude about gene technology. The Pharmacogenetic Phase I Unit population can then be used to test for the existence of genetic variation in response to any drug as a first step in deciding whether to proceed with extensive pharmacogenetic studies in later stages of clinical development. Specific uses of a large Phase I unit in which some or all subjects are related include:

a. It should be possible, for virtually any compound, to assess the magnitude of the genetic contribution to variation in drug response (if any) by comparing variation in drug response traits among related vs. non-related individuals. The rationale is as follows: if a surrogate drug response trait (i.e., a surrogate marker of pharmacodynamic effect that can be measured in normal subjects) is under strong genetic control then related individuals, who share 25% (cousins) or 50% (sibs) of their alleles, should have less divergent responses (less intragroup variance) than unrelated individuals, who share a much smaller fraction of alleles. That is, individuals who share alleles at the genes that affect drug response should be more similar to each other (i.e. have a narrower distribution of responses, whether measured by variance, standard deviation or other means) than individuals who, on average, share very few alleles. By using statistical methods known in the art the degree of variation in a set of data from related individuals (each individual would only be compared with his/her relatives, but such comparisons would be performed within each group of relatives and a summary statistic developed) could be compared to the degree of variation in a set of unrelated individuals (the same subjects could be used, but the second comparison would be across related groups). Account would be taken of the degree of similarity expected between related individuals, based on the fraction of the genome they shared by descent. Thus the extent of variation in the surrogate response marker between identical twins should be less than between sibs, which should be less than between first cousins, which should be less than that between second cousins, and so forth, if there is a genetic component to the variation. It is well known from twin studies (in which, for example, variation between identical twins is compared to variation between fraternal twins) that pharmacokinetic variables (e.g. compound half life, peak concentration) are frequently over 90% heritable; the type of study proposed here (comparison of variation within groups of sibs and cousins to variation between unrelated subjects) would also show this genetic effect, without requiring the recruitment of monozygotic twins. For a summary of pharmacokinetic studies in twins see: Propping, Paul (1978) Pharmacogenetics. Rev. Physiol. Biochem. Pharmacol. 83: 123-173.

It may be that the pattern of drug responses that distinguishes related individuals from non-related individuals is more complex than, for example, variance or standard deviation. For example, there may be two discrete phenotypes characteristic of intrafamilial variation (a bimodal distribution) that are not a feature of variation between unrelated individuals (where, for example, variation might be more nearly continuous). Such a pattern could be attributable to Mendelian inheritance operating on a restricted set of alleles in a family (or families) with, for example, AA homozygotes giving one phenotype and AB heterozygotes and BB homozygotes giving a second phenotype, all in the context of a relatively homogeneous genetic background. In contrast, variation among non-related subjects would be less discrete due to a greater degree of variation in genetic background and the presence of additional alleles C, D and E at the candidate locus. Statistical measures of the significance of such differences in distribution, including nonparametric methods such as chi square and contingency tables, are known in the art.

The methods described herein for measuring whether pharmacodynamic traits are under genetic control, using surrogate markers of drug efficacy in phase I studies which include groups of related individuals, will be useful in obtaining an early assessment of the extent of genetically determined variation in drug response for a given therapeutic compound. Such information provides an informed basis for either stopping development at the earliest possible stage or, preferably, continuing with development but with a plan for identifying and controlling for genetic variation so as to allow rapid progression through the regulatory approval process.

For example, it is well known that Alzheimer's trials are long and expensive, and most drugs are only effective in a fraction of patients. Using surrogate measures of response in normals drawn from a population of related individuals would help to assess the contribution of genetic variation to variation in treatment response. For an acetylcholinesterase inhibitor, relevant surrogate pharmacodynamic measures could include testing erythrocyte membrane acetylcholinesterase levels in drug treated normal subjects, or performing psychometric tests that are affected by treatment (and ideally that correlate with clinical efficacy) and measuring the effect of treatment. As another example, antidepressant drugs can produce a variety of effects on mood in normal subjects—or no effect at all. Careful monitoring and measurement of such responses in related vs. unrelated normal subjects, and statistical comparison of the degree of variation in each group, could provide an early readout on whether there is a genetic component to drug response (and hence clinical efficacy). The observation of similar effects in family members, and comparatively dissimilar effects in unrelated subjects would provide compelling evidence of a pharmacogenetic effect and justify the substantial expenditure necessary for a full pharmacogenetic drug development program. Conversely, the absence of any significant family influence on drug response would provide an early termination point for pharmacogenetic studies. Note that the proposed studies do not require any knowledge of candidate genes, nor is DNA collection or genotyping required—simply a reliable surrogate pharmacodynamic assay and small groups of related normal individuals. Refined statistical methods should permit the magnitude of the pharmacogenetic effect to be measured, which could be a further criteria for deciding whether to proceed with pharmacogenetic analysis. The greater the differential in magnitude or pattern of variance between the related and the unrelated subjects, the greater the extent of genetic control of the trait.

Not all drug response traits are under the predominant control of one locus. Many such traits are under the control of multiple genes, and may be referred to as quantitative trait loci. It is then desirable to identify the major loci contributing to variation in the drug response trait. This can be done for example, to map quantitative trait loci in a population of drug treated related normals. Either a candidate gene approach or a genome wide scanning approach can be used. (For review of some relevant methods see: Hsu L, Aragaki C, Quiaoit F. (1999) A genome-wide scan for a simulated data set using two newly developed methods. Genet Epidemiol 17 Suppl 1:S621-6; Zhao L P , Aragaki C, Hsu L, Quiaoit F. (1998) Mapping of complex traits by single-nucleotide polymorphisms. Am J Hum Genet 63(l):225-40; Stoesz M R, Cohen J C, Mooser V, et al. (1997) Extension of the Haseman-Elston method to multiple alleles and multiple loci: theory and practice for candidate genes. Ann Hum Genet 61 (Pt 3):263-74.)) However, this method would require at least 100 patients (preferably 200, and still more preferably >300) to have adequate statistical power, and each patient would have to be genotyped at a few polymorphic loci (candidate gene approach) or hundreds of polymorphic loci (genome scanning approach).

b. With a large Phase I population of normal subjects that need not be related (a second type of Pharmacogenetic Phase I Unit) it is possible to efficiently identify and recruit for any Phase I trial a set of individuals comprising virtually any combination of genotypes present in a population (for example, all common genotypes, or a group of genotypes expected to represent outliers for a drug response trait of interest). This method preferably entails obtaining blood or other tissue (e.g. buccal smear) in advance from a large number of the subjects in the Phase I unit. Ideally consent for genotyping would be obtained at the same time. It would be most efficient if blanket consent for genotyping any polymorphic site or sites could be obtained. Second best would be consent for testing any site relevant to any customer project (not specific at the time of initial consent). Third best would be consent to genotype polymorphic sites relevant to specific disease areas. Another, less desirable, solution would be to obtain consent for genotyping on a project by project basis (for example by mailing out reply cards), after the specific polymorphic sites to be genotyped are known.

One useful way to screen for pharmacogenetic effects in Phase I is to recruit homozygotes for a variance or variances of interest in one or more candidate genes. For example, consider a compound for which there are two genes that are strong candidates for influencing response to treatment. Gene X has alleles A and A′, while gene Y has alleles B and B′. If these genes do in fact contribute significantly to response then one would expect that, regardless of the mode of inheritance (recessive, codominant, dominant, polygenic) homozygotes would exhibit the most extreme responses. One would also expect epistatic interactions, if any, to be most extreme in double homozygotes. Thus one would ideally perform a surrogate drug response test in Phase I volunteers doubly homozygous at both X and Y. That is, test AA/BB, A′A′/BB, AA/B′B′ and A′A′/′B′ subjects. If the allele frequencies for A and A′ are 0.15 and 0.85, and for B and B′ 0.2 and 0.8 then the frequency of AA homozygotes is expected to be 2.25% and BB homozygotes 4%. In the absence of any linkage between the genes, the frequency of AA/BB double homozygotes is expected to be 0.0225×0.04=0.0009 or 0.09%, or about 1 subject in 1000. Ideally at least 5 subjects of each genotype are recruited for the Phase I study, and preferably at least 10 subject. Thus, even for variances of moderately low allele frequency (15%, 20%), the identification of potential outliers (i.e. homozygotes) for the candidate genes of interest will require a large population. Preferably the Phase I unit has enrolled at least 1,000 normal individuals, more preferably 2,000, still more preferably 5,000 and most preferably 10,000 or more. In another application of the large, genotyped Phase I population it may be useful to identify individuals with rare variances in candidates genes (either homozygous or heterozygous), in order to determine whether those variances are predisposing to extreme pharmacological responses to the compound. For example, variances occurring at 5% allele frequency are expected to occur in homozygous form in 0.25% of the population (0.05×0.05), and therefore may rarely, if ever, be encountered in early clinical development. Yet it may be serious adverse effects occurring in just such a small group that create problems in later stages of drug development. In yet another application of the large genotyped Phase I population, subjects may be selected to represent the known common variances in one or more genes that are candidates for influencing the response to treatment. By insuring that all common genotypes are represented in a Phase I trial the likelihood of misleading results due to genetic stratification (resulting in discrepancy with results of later, larger trials can be reduced.

It would be useful to prospectively genotype the large Phase I population for variances that are commonly the source of interpatient variation in drug response, since demand for genotyped groups of such patients can be anticipated from pharmaceutical companies and contract research organizations (CROs). For example, genotyping might initially focus on common pharmacological targets such as estrogen receptors, adrenergic receptors, or serotonin receptors. The pre-genotyped Phase I population could be part of a package of services (along with genotyping assay development capability, high throughput genotyping capacity and software and expertise in statistical genetics) designed to accelerate pharmacogenetic Phase I studies. Eventually, as the databank of genotypes built up, individuals with virtually any genotype or combination of genotypes could be called in for precisely designed physiological or toxicological studies designed to test for pharmacogenetic effects.

One of the most useful aspects of the Pharmacogenetic Phase I Unit is that subjects with rare genotypes can be pharmacologically assessed in a small study. This addresses a serious limitation of conventional clinical trials with respect to the investigation of polygenic traits or the effect of rare alleles. Unfortunately even Phase III studies, as currently performed, are often barely powered to address simple one variance hypotheses about efficacy or toxicity. The problem, of course, is that each time a new genetic variable is introduced the comparison groups are cut in halves or thirds (or even smaller groups if there are multiple haplotypes at each gene). It is therefore a challenging problem to test the interaction of several genes in determining drug response. Yet the character of drug response data in populations—there is often a continuous distribution of responses among different individuals—suggests that drug responses may often be mediated by several genes. (On the other hand, there are an increasing number of well documented single gene, or even single variance, pharmacogenetic effects in the literature, showing that it is possible to detect the effect of a single variance.) One approach to identifying pharmacogenetic effects is to focus on finding the single gene variances that have the largest effects. This approach can be undertaken within the scale of current clinical trials. However, in order to develop a test which predicts a large fraction of the quantitative variation in a drug response trait it may be desirable to test the effect of multiple genes, including the interaction of variances at different genes, which may be non-additive (referred to as epistasis). The Pharmacogenetic Phase I Unit provides a way to efficiently test for gene interactions or multigene effects by, for example, allowing easy identification of individuals who, on account of being homozygous at several loci of interest, should be outliers for the drug response phenotypes of interest if there is a gene×gene interaction. Testing drug response in a small number of such individuals will provide a quick read on gene interaction. Obtaining genetic data on the pharmacodynamic action of a compound in Phase I should also provide a crude measure of allele affects—which variances or haplotypes increase pharmacological responses and which decrease them. This information is of great value in designing subsequent trials, as it constrains the number of hypotheses to be tested, thereby enabling powerful statistical designs. This is because when the effect of variances on drug response measures is unknown one is forced to statistically test all the possible effects of each allele (e.g. two tailed tests). As the number of genetically defined groups increases (e.g. as a result of multiple variances or haplotypes) there is a loss of statistical power due to multiple testing correction. On the other hand, if the relative phenotypic effect of each allele at a locus is known (or can be hypothesized) from Phase I data then each individual in a subsequent clinical trial contributes useful information—there is a specific prediction of response based on that individuals combination of genotypes or haplotypes, and testing the fit of the actual data to those predictions provides for powerful statistical designs. (It is also possible to measure allele effects biochemically, of course, to establish which alleles have positive and which negative effects, but at considerable cost.)

It is important to note that Phase I trials can provide useful information at almost any stage of clinical development. It is not unusual, for example, for a product in Phase II or even Phase III testing to be remanded to Phase I in order to clarify some aspect of toxicology or physiology. In this context a Pharmacogenetic Phase I Unit would be extremely useful to a drug development company. Phase I studies in defined genetic subgroups drawn from a large genotyped population, or in groups of related individuals, would be the most economical and efficient way to clarify the existence of pharmacogenetic effects, if any, paving the way for future rational development of the product.

C. Phase II Clinical Trials

Phase II studies generally include a limited number of patients (<100) who satisfy a set of predefined inclusion criteria and do not satisfy any predefined exclusion criteria of the trial protocol. Phase II studies can be conducted at single or multiple institutions. Inclusion/exclusion criteria may include historical, clinical and laboratory parameters for a disease, disorder, or condition; age; gender; reproductive status (i.e. pre- or postmenopausal); coexisting medical conditions; psychological, emotional or cognitive state, or other objective measures known to those skilled in the art. In a pharmacogenetic Phase II trial the inclusion/exclusion criteria may include one or more genotypes or haplotypes. Alternatively, genetic analysis may be performed at the end of the trial. The primary goals in Phase II testing may include (i) identification of the optimal medical indication for the compound, (ii) definition of an optimal dose or range or doses, balancing safety and efficacy considerations (dose-finding studies), (iii) extended safety studies (complementing Phase I safety studies), (iv) evaluation of efficacy in patients with the targeted disease or condition, either in comparison to placebo or to current best therapy. To some extent these goals may be achieved by performing multiple trials with different goals. Likewise, Phase II trials may be designed specifically to evaluate pharmacogenetic aspects of the drug candidate. Primary efficacy endpoints typically focus on clinical benefit, while surrogate endpoints may measure treatment response variables such as clinical or laboratory parameters that track the progress or extent of disease, often at lesser time, cost or difficulty than the definitive endpoints. A good surrogate marker must be convincingly associated with the definitive outcome. Examples of surrogate endpoints include tumor size as a surrogate for survival in cancer trials, and cholesterol levels as a surrogate for heart disease (e.g. myocardial infarction) in trials of lipid lowering cardiovascular drugs. Secondary endpoints supplement the primary endpoint and may be selected to help guide further clinical studies.

In a pharmacogenetic Phase II clinical trial, retrospective or prospective design will include the stratification of patients based upon a variance or variances in a gene or genes suspected of affecting treatment response. The gene or genes may be involved in mediating pharmacodynamic or pharmacokinetic response to the candidate therapeutic intervention. The parameters evaluated in the genetically stratified trial population may include primary, secondary or surrogate endpoints. Pharmacokinetic parameters—for example, dosage, absorption, toxicity, metabolism, or excretion—may also be evaluated in genetically stratified groups.. Other parameters that may be assessed in parallel with genetic stratification include gender, race, ethnic or geographic origin (population history) or other demographic factors.

While it is optimal to initiate pharmacogenetic studies in phase I, as described above, it may be the case that pharmacogenetic studies are not considered until phase II, when problems relating either to efficacy or toxicity are first encountered. It is highly desirable to initiate pharmacogenetic studies no later than Phase II of a clinical development plan because (1) phase III studies tend to be large and expensive—not an optimal setting in which to explore untested pharmacogenetic hypotheses; (2) phase III studies are typically designed to test one fairly narrow hypothesis regarding efficacy of one or a few dose levels in a specific disease or condition. Phase II studies are often numerous, and are intended to provide a broad picture of the pharmacology of the candidate compound. This is a good setting for initial pharmacogenetic studies. Several pharmacogenetic hypotheses may be tested in phase II, with the goal of eliminating all but one or two.

D. Phase III Clinical Trials

Phase III studies are generally designed to measure efficacy of a new treatment in comparison to placebo or to an established treatment method. Phase II studies are often performed at multiple sites. The design of this type of trial includes power analysis to ensure the sufficient data will be gathered to demonstrate the anticipated effect, making assumptions about response rate based on earlier trials. As a result Phase III trials frequently include large numbers of patients (up to 5,000). Primary endpoints in Phase III studies may include reduction or arrest of disease progression, improvement of symptoms, increased longevity or increased disease-free longevity, or other clinical measures known in the art. In a pharmacogenetic Phase III clinical study, the endpoints may include determination of efficacy or toxicity in genetically defined subgroups. Preferably the genetic analysis of outcomes will be confined to an assessment of the impact of a small number of variances or haplotypes at a small number of genes, said variances having already been statistically associated with outcomes in earlier trials. Most preferably variances at only one or two genes will be assessed.

After successful completion of one or more Phase III studies, the data and information from all trials conducted to test a new treatment method are compiled into a New Drug Application (NDA) and submitted for review by the U.S. FDA, which has authority to grant marketing approval in the U.S. and its territories. The NDA includes the raw (unanalyzed) clinical data, i.e. the patient by patient measurements of primary and secondary endpoints, a statistical analysis of all of the included data, a document describing in detail any observed side effects, tabulation of all patients who dropped-out of trials and detailed reasons for their termination, and any other available data pertaining to ongoing in vitro or in vivo studies since the submission of the investigational new drug (IND) application. If pharmacoeconomic objectives are a part of the clinical trial design then data supporting cost or economic analyses are included in the NDA. In a pharmacogenetic clinical study, the pharmacoeconomic analyses may include genetically stratified assessment of the candidate therapeutic intervention in a cost benefit analysis, cost of illness study, cost minimization study, or cost utility analysis. The analysis may also be simultaneously stratified by standard criteria such as race/ethnicity/geographic origin, sex, age or other criteria. Data from a genetically stratified analysis may be used to support an application for approval for marketing of the candidate therapeutic intervention.

E. Phase IV Clinical Trials

Phase IV studies occur after a therapeutic intervention has been approved for marketing, and are typically conducted for surveillance of safety, particularly occurrence of rare side effects. The other principal reason for Phase IV studies is to produce information and relationships useful for marketing a drug. In this regard pharmacogenetic analysis may be very useful in Phase IV trials. Consider, for example, a drug that is the fourth or fifth member of a drug class (say statins, or thiazidinediones or fluoropyrimidines) to obtain marketing approval, and which does not differ significantly in clinical effects—efficacy or safety—from other members of the drug class. The first, second and third drugs in the class will likely have a dominant market position (based on their earlier introduction into the marketplace) that is difficult to overcome, particularly in the absence of differentiating clinical effects. However, it is possible that the new drug produces a superior clinical effect—for example, higher response rate, greater magnitude of response or fewer side effects—in a genetically defined subgroup. The genetic subgroup with superior response may constitute a larger fraction of the total patient population than the new drug would likely achieve otherwise. In this instance, there is a clear rationale for performing a Phase IV pharmacogenetic trial to identify a variance or variances that mark a patient population with superior clinical response. Subsequently a marketing campaign can be designed to alert patients, physicians, pharmacy managers, managed care organizations and other parties that, with the use of a rapid and inexpensive genetic test to identify eligible patients, the new drug is superior to other members of the class (including the market leading first, second and third drugs introduced). The high responder subgroup defined by a variance or variances may also exhibit a superior response to other drugs in the class (a class pharmacogenetic effect), or the superior efficacy in the genetic subgroup may be specific to the drug tested (a compound-specific pharmacogenetic effect).

In a Phase IV pharmacogenetic clinical trial, both retrospective and prospective analysis can be performed. In both cases, the key element is genetic stratification based on a variance or variances or haplotype. Phase IV trials will often have adequate sample size to test more than one pharmacogenetic hypothesis in a statistically sound way.

F. Unconventional Clinical Development

Although the above listed phases of clinical development are well-established, there are cases where strict Phase I, II, III development does not occur, for example, in the clinical development of candidate therapeutic interventions for debilitating or life threatening diseases, or for diseases where there is presently no available treatment. Some of the mechanisms established by the FDA for such studies include Treatment INDs, Fast-Track or Accelerated reviews, and Orphan Drug Status. In a clinical development program for a candidate therapeutic of this type there is a useful role for pharmacogenetic analysis, in that the candidate therapeutic may not produce a sufficient benefit in all patients to justify FDA approval, however analysis of outcome in genetic subgroups may lead to identification of a variance or variances that predict a response rate sufficient for FDA approval.

As used herein, “supplemental applications” are those in which a candidate therapeutic intervention is tested in a human clinical trial in order to gain an expanded label indication, expanding recommended use to new medical indications. In these applications, previous clinical studies of the therapeutic intervention, i.e. preclinical safety and Phase I human safety studies can be used to support the testing of the therapeutic intervention in a new indication. Pharmacogenetic analysis is also useful in the context of clinical trials to support supplemental applications. Since these are, by definition, focused on diseases not selected for initial development the overall efficacy may not be as great as for the leading indication(s). The identification of genetic subgroups with high response rates may enable the rapid approval of supplemental applications for expanded label indications. In such instances part of the label indication may be a description of the variance or variances that define the group with superior response.

As used herein, “outcomes” or “therapeutic outcomes” describe the results and value of healthcare intervention. Outcomes can be multi-dimensional, and may include improvement of symptoms; regression of a disease, disorder, or condition; prevention of a disease or symptom; cost savings or other measures.

Pharmacoeconomics is the analysis of a therapeutic intervention in a population of patients diagnosed with a disease, disorder, or condition that includes at least one of the following studies: cost of illness study (COI); cost benefit analysis (CBA), cost minimization analysis (CMA), or cost utility analysis (CUA), or an analysis comparing the relative costs of a therapeutic intervention with one or a group of other therapeutic interventions. In each of these studies, the cost of the treatment of a disease, disorder, or condition is compared among treatment groups. Costs have both direct (therapeutic interventions, hospitalization) and indirect (loss of productivity) components. Pharmacoeconomic factors may provide the motivation for pharmacogenetic analysis, particularly for expensive therapies that benefit only a fraction of patients. For example, interferon alpha is the only treatment that can cure hepatitis C virus infection, however viral infection is completely and permanently eliminated in less than a quarter of patients. Nearly half of patients receive virtually no benefit from alfa interferon, but may suffer significant side effects. Treatment costs are ˜$10,000 per course. A pharmacogenetic test that could predict responders would save much of the cost of treating patients not able to benefit from interferon alpha therapy, and could provide the rationale for treating a population in a cost efficient manner, where treatment would otherwise be unaffordable.

As used herein, “health-related quality of life” is a measure of the impact of a disease, disorder, or condition on a patient's activities of daily living. An analysis of the health-related quality of life is often included in pharmacoeconomic studies.

As used herein, the term “stratification” refers to the partitioning of patients into groups on the basis of clinical or laboratory characteristics of the patient. “Genetic stratification” refers to the partitioning of patients or normal subjects into groups based on the presence or absence of a variance or variances in one or more genes. The stratification may be performed at the end of the trial, as part of the data analysis, or may come at the beginning of a trial, resulting in creation of distinct groups for statistical or other purposes.

G. Power Analysis in Pharmacogenetic Clinical Trials

The basic goal of power calculations in clinical trial design is to insure that trials have adequate patients and controls to fairly assess, with statistical significance, whether the candidate therapeutic intervention produces a clinically significant benefit.

Power calculations in clinical trials are related to the degree of variability of the drug response phenotypes measured and the treatment difference expected between comparison groups (e.g. between a treatment group and a control group). The smaller the variance within each group being compared, and the greater the difference in response between the two groups, the fewer patients are required to produce convincing evidence of an effect of treatment. These two factors (variance and treatment difference) determine the degree of precision required to answer a specific clinical question.

The degree of precision may be expressed in terms of the maximal acceptable standard error of a measurement, the magnitude of variation in which the 95% confidence interval must be confined or the minimal magnitude of difference in a clinical or laboratory value that must be detectable (at a statistically significant level, and with a specified power for detection) in a comparison to be performed at the end of the trial (hypothesis test). The minimal magnitude is generally set at the level that represents the minimal difference that would be considered of clinical importance.

In pharmacogenetic clinical trials there are two countervailing effects with respect to power. First, the comparison groups are reduced in size (compared to a conventional trial) due to genetic partitioning of both the treatment and control groups into two or more subgroups. However, it is reasonable to expect that variability for a trait is smaller within groups that are genetically homogeneous with respect to gene variances affecting the trait. If this is the case then power is increased as a function of the reduction in variability within (genetically defined) groups.

In general it is preferable to power a pharmacogenetic clinical trial to see an effect in the largest genetically defined subgroups. For example, for a variance with allele frequencies of 0.7 and 0.3 the common homozygote group will comprise 49% of all patients (0.7×0.7×100). It is most desirable to power the trial to observe an effect (either positive or a negative) in this group. If it is desirable to measure an effect of therapy in a small genetic group (for example, the 9% of patients homozygous for the rare allele) then genotyping should be considered as an enrollment criterion to insure a sufficient number of patients are enrolled to perform an adequately powered study.

Statistical methods for powering clinical trials are known in the art. See, for example: Shuster, J. J. (1990) Handbook of Sample Size Guidelines for Clinical Trials. CRC Press, Boca Raton, Fla.; Machin, D. and M. J. Campbell (1987) Statistical Tables for the Design of Clinical Trials. Blackwell, Oxford, UK; Donner, A. (1984) Approaches to Sample Size Estimation in the Design of Clinical Trials—A Review. Statistics in Medicine 3: 199-214.

H. Statistical Analysis of Clinical Trial Data

There are a variety of statistical methods for measuring the difference between two or more groups in a clinical trial. One skilled in the art will recognize that different methods are suited to different data sets. In general, there is a family of methods customarily used in clinical trials, and another family of methods customarily used in genetic epidemiological studies. Methods in quantitative and population genetics designed to measure the association between genotypes and phenotypes, and to map and measure the effect of quantitative trait loci are also relevant to the task of measuring the impact of a variance on response to a treatment. Methods from any of these disciplines may be suitable for performing statistical analysis of pharmacogenetic clinical trial data, as is known to those skilled in the art.

Conventional clinical trial statistics include hypothesis testing and descriptive methods, as elaborated below. Guidance in the selection of appropriate statistical tests for a particular data set is provided in texts such as: Biostatistics: A Foundation for Analysis in the Health Sciences, 7th edition (Wiley Series in Probability and Mathematical Statistics, Applied Probability and statistics) by Wayne W. Daniel, John Wiley & Sons, 1998; Bayesian Methods and Ethics in a Clinical Trial Design (Wiley Series in Probability and Mathematical Statistics. Applied Probability Section) by J. B. Kadane (Editor), John Wiley & Sons, 1996. Examples of specific hypothesis testing and descriptive statistical procedures that may be useful in analyzing clinical trial data are listed below.

A. Hypothesis Testing Statistical Procedures

(1) One-sample procedures (binomial confidence interval, Wilcoxon signed rank test, permutation test with general scores, generation of exact permutational distributions)

(2) Two-sample procedures (t-test, Wilcoxon-Mann-Whitney test, Normal score test, Median test, Van der Waerden test, Savage test, Logrank test for censored survival data, Wilcoxon-Gehan test for censored survival data, Cochran-Armitage trend test, permutation test with general scores, generation of exact permutational distributions)

(3) R×C contingency tables (Fisher's exact test, Pearson's chi-squared test, Likelihood ratio test, Kruskal-Wallis test, Jonckheere-Terpstra test, Linear-by linear association test, McNemar's test, marginal homogeneity test for matched pairs)

(4) Stratified 2×2 contingency tables (test of homogeneity for odds ratio, test of unity for the common odds ratio, confidence interval for the common odds ratio)

(5) Stratified 2×C contingency tables (all two-sample procedures listed above with stratification, confidence intervals for the odds ratios and trend, generation of exact permutational distributions)

(6) General linear models (simple regression, multiple regression, analysis of variance —ANOVA—, analysis of covariance, response-surface models, weighted regression, polynomial regression, partial correlation, multiple analysis of variance —MANOVA—, repeated measures analysis of variance).

(7) Analysis of variance and covariance with a nested (hierarchical) structure.

(8) Designs and randomized plans for nested and crossed experiments (completely randomized design for two treatment, split-splot design, hierarchical design, incomplete block design, latin square design)

(9) Nonlinear regression models

(10) Logistic regression for unstratified or stratified data, for binary or ordinal response data, using the logit link function, the normit function or the complementary log-log function.

(11) Probit, logit, ordinal logistic and gompit regression models.

(12) Fitting parametric models to failure time data that may be right-, left-, or interval-censored. Tested distributions can include extreme value, normal and logistic distributions, and, by using a log transformation, exponential, Weibull, lognormal, loglogistic and gamma distributions.

(13) Compute non-parametric estimates of survival distribution with right-censored data and compute rank tests for association of the response variable with other variables.

B. Descriptive Statistical Methods

Factor analysis with rotations

Canonical correlation

Principal component analysis for quantitative variables.

Principal component analysis for qualitative data.

Hierarchical and dynamic clustering methods to create tree structure, dendrogram or phenogram.

Simple and multiple correspondence analysis using a contingency table as input or raw categorical data.

Specific instructions and computer programs for performing the above calculations can be obtained from companies such as: SAS/STAT Software, SAS Institute Inc., Cary, N.C., U.S.A; BMDP Statistical Software, BMDP Statistical Software Inc., Los Angeles, Calif., USA; SYSTAT software, SPSS Inc., Chicago, Ill., USA; StatXact & LogXact, CYTEL Software Corporation, Cambridge, Mass., USA.

C. Statistical Genetic Methods Useful for Analysis of Pharmacogenetic Data

A wide spectrum of mathematical and statistical tools may be useful in the analysis of data produced in pharmacogenetic clinical trials, including methods employed in molecular, population, and quantitative genetics, as well as genetic epidemiology. Methods developed for plant and animal breeding may be useful as well, particularly methods relating to the genetic analysis of quantitative traits.

Analytical methods useful in the analysis of genetic variation among individuals, populations and species of various organisms are described in the following texts: Molecular Evolution, by W- H. Li, Sinauer Associates, Inc., 1997; Principles of Population Genetics, by D. L. Hartl and A. G. Clark, 1996; Genetics and Analysis of Quantitative Traits, By M. Lynch and B. Walsh, Sinauer Associates, Inc., Principles of Quantitative Genetics, by D. S. Falconer and T. F. C. Mackay, Longman, 1996; Genetic Variation and Human Disease, by K. M. Weiss, Cambridge University Press, 1993; Fundamentals of Genetic Epidemiology, by M. J. Khoury, T. H. Beaty, and B. H. Cohen, Oxford University Press, 1993; Handbook of Genetic Linkage, by J. Terwilliger J. Ott, Johns Hopkins University Press, 1994.

The types of statistical analysis performed in different branches of genetics are outlined below as a guide to the relevant literature and publicly available software, some of which is cited.

Molecular Evolutionary Genetics

Patterns of nucleotide variation among individuals, families/populations and across species and genera,

Alignment of sequences and description of variation/polymorphisms among the aligned sequences, amounts of similarities and dissimilarities,

Measurement of molecular variation among various regions of a gene, testing of neutrality models,

Rates of nucleotide changes among coding and the non-coding regions within and among populations,

Construction of phylogenetic trees using methods such as neighborhood joining and maximum parsimony; estimation of ages of variances using coalescent models,

Population Genetics

Patterns of distribution of genes among genotypes and populations. Hardy-Weinberg equilibrium, departures form the equilibrium

Genotype and haplotype frequencies, levels of heterozygosities, polymorphism information contents of genes, estimation of haplotypes from genotypes; the E-M algorithm, and parsimony methods

Estimation of linkage disequilibrium and recombination

Hierarchical structure of populations, the F-statistics, estimation of inbreeding, selection and drift

Genetic admixture/migration and mutation frequencies

Spatial distribution of genotypes using spatial autocorrelation methods

Kin-structured maintenance of variation and migration

Quantitative Genetics

Phenotype as the product of the interaction between genotype and environment

Additive, dominance and epistatic variance on the phenotype

Effects of homozygosity, heterozygosity and developmental homeostasis

Estimation of heritability: broad sense and narrow sense

Determination of number of genes governing a character

Determination of quantitative trait loci (QTLs) using family information or population information, and using linkage and/or association studies

Determination of quantitative trait nucleotide (QTN) using a combination linkage disequilibrium methods and cladistic approaches

Determination of individual causal nucleotide in the diploid or haploid state on the phenotype using the method of measured genotype approaches, and combined effects or synergistic interaction of the causal mutations on the phenotype

Determination of relative importance of each of the mutations on a given phenotype using multivariate methods, such as discriminant function, principal component and step-wise regression methods

Determination of direct and indirect effect of polymorphisms on a complex phenotype using path analysis (partial regression ) methods

Determination of the effects of specific environment on a given genotype—genotype×environment interactions using joint regression and additive and multiplicative parameter methods.

Genetic Epidemiology

Determination of sample size based on the disease and the marker frequency in the “case” and in the “control” populations

Stratification of study population based on gender, ethnic, socio-economic variation

Establishing a “causal relationship” between genotype and disease, using, using various association and linkage approaches—viz., case-control designs, family studies (if available), transmission disequilibrium tests etc.,

Linkage analysis between markers and a candidate locus using two-point and multipoint approaches. Computer programs used for genetic analysis are: Dna SP version 3.0, by Juilo Rozas, University of Barcelona, Spain. Http://www.bio.ub.es/-Julio; Arlequin 1.1 by S. Schnieder, J -M Kueffer, D. Roessli and L. Excoffier. University of Geneva, Switzerland, http://anthropologie.unige.ch/arlequin. PAUP*4, by D. L. Swofford, Sinauer Associates, Inc., 1999. SYSTAT software, SPSS Inc., Chicago, Ill., 1998; . Linkage User's Guide, by J. Ott, Rockefeller University, Http://Linkage.rockefeller.edu/soft/linkage

Guidance in the selection of appropriate genetic statistical tests for analysis of data can be obtained from texts such as: Fundamentals of Genetic Epidemiology (Monographs in Epidemiology and Biostatistics, Vol 22) by M. J. Khoury, B. H. Cohen & T. H. Beaty, Oxford Univ Press, 1993; Methods in Genetic Epidemiology by Newton E. Morton, S. Karger Publishing, 1983; Methods in Observational Epidemiology, 2nd edition (Monographs in Epidemiology and Biostatistics, V. 26) by J. L. Kelsey (Editor), A. S. Whittemore & A. S. Evans, 1996; Clinical Trials: Design, Conduct, and Analysis (Monographs in Epidemiology and Biostatistics, Vol 8) by C. L. Meinert & S. Tonascia, 1986)

I. Retrospective Clinical Trials

In general the goal of retrospective clinical trials is to test and refine hypotheses regarding genetic factors that are associated with drug responses. The best supported hypotheses can subsequently be tested in prospective clinical trials, and data from the prospective trials will likely comprise the main basis for an application to register the drug and predictive genetic test with the appropriate regulatory body. In some cases, however, it may become acceptable to use data from retrospective trials to support regulatory filings. Exemplary strategies and criteria for stratifying patients in a retrospective clinical trial are provided below.

Clinical Trials to Study the Effect of One Gene Locus on Drug Response

A. Stratify Patients by Genotype at One Candidate Variance in the Candidate Gene Locus

1. Genetic stratification of patients can be accomplished in several ways, including the following (where ‘A’ is the more frequent form of the variance being assessed and ‘a’ is the less frequent form):

(a) AA vs. aa

(b) AA vs. Aa vs. aa

(c) AA vs. (Aa+aa)

(d) (AA+Aa) vs. aa.

2. The effect of genotype on drug response phenotype may be affected by a variety of nongenetic factors. Therefore it may be beneficial to measure the effect of genetic stratification in a subgroup of the overall clinical trial population. Subgroups can be defined in a number of ways including, for example, biological, clinical, pathological or environmental criteria. For example, the predictive value of genetic stratification can be assessed in a subgroup or subgroups defined by:

a. Biological Criteria

i. gender (males vs. females)

ii. age (for example above 60 years of age). Two, three or more age groups may be useful for defining subgroups for the genetic analysis.

iii. hormonal status and reproductive history, including pre- vs. post-menopausal status of women, or multiparous vs. nulliparous women

iv. ethnic, racial or geographic origin, or surrogate markers of ethnic, racial or geographic origin. (For a description of genetic markers that serve as surrogates of racial/ethnic origin see, for example: Rannala, B. and J. L. Mountain, Detecting immigration by using multilocus genotypes. Proc Natl Acad Sci U S A , 94 (17): 9197-9201, 1997. Other surrogate markers could be used, including biochemical markers.)

b. Clinical Criteria

i. Disease status. There are clinical grading scales for many diseases. For example, the status of Alzheimer's Disease patients is often measured by cognitive assessment scales such as the mini-mental status exam (MMSE) or the Alzheimer's Disease Assessment Scale (ADAS), which includes a cognitive component (ADAS-COG). There are also clinical assessment scales for many other diseases, including cancer.

ii. Disease manifestations (clinical presentation).

iii. Radiological staging criteria.

c. Pathological criteria:

i. Histopathologic features of disease tissue, or pathological diagnosis. (For example there are many varieties of lung cancer: squamous cell carcinoma, adenocarcinoma, small cell carcinoma, bronchoalveolar carcinoma, etc., each of which may—which, in combination with genetic variation, may correlate with

ii. Pathological stage. A variety of diseases, particularly cancer, have pathological staging schemes

iii. Loss of heterozygosity (LOH)

iv. Pathology studies such as measuring levels of a marker protein

v. Laboratory studies such as hormone levels, protein levels, small molecule levels

3. Measure frequency of responders in each genetic subgroup. Subgroups may be defined in several ways.

i. more than two age groups

ii. reproductive status such as pre or post-menopausal

4. Stratify by haplotype at one candidate locus where the haplotype is made up of two variances, three variances or greater than three variances.

Data from already completed clinical trials can be retrospectively reanalyzed. Since the questions are new, the data can be treated as if it were a prospective trial, with identified variances or haplotypes as stratification criteria or endpoints in clinically stratified data (e.g. what is the frequency of a particular variance in a response group compared to nonresponsders). Care should be taken to in studying a population in which there may be a link between drug-related genes and disease-related genes.

Retrospective pharmacogenetic trials can be conducted at each of the phases of clinical development, if sufficient data is available to correlate the physiologic effect of the candidate therapeutic intervention and the allelic variance or variances within the treatment population. In the case of a retrospective trial, the data collected from the trial can be re-analyzed by imposing the additional stratification on groups of patients by specific allelic variances that may exist in the treatment groups. Retrospective trials can be useful to ascertain whether a hypothesis that a specific variance has a significant effect on the efficacy or toxicity profile for a candidate therapeutic intervention.

A prospective clinical trial has the advantage that the trial can be designed to ensure the trial objectives can be met with statistical certainty. In these cases, power analysis, which includes the parameters of allelic variance frequency, number of treatment groups, and ability to detect positive outcomes can ensure that the trial objectives are met.

In designing a pharmacogenetic trial, retrospective analysis of Phase II or Phase III clinical data can indicate trial variables for which further analysis is beneficial. For example, surrogate endpoints, pharmacokinetic parameters, dosage, efficacy endpoints, ethnic and gender differences, and toxicological parameters may result in data that would require further analysis and re-examination through the design of an additional trial. In these cases, analysis involving statistics, genetics, clinical outcomes, and economic parameters may be considered prior to proceeding to the stage of designing any additional trials. Factors involved in the consideration of statistical significance may include Bonferroni analysis, permutation testing, with multiple testing correction resulting in a difference among the treatment groups that has occurred as a result of a chance of no greater than 20%, i.e. p<0.20. Factors included in determining clinical outcomes to be relevant for additional testing may include, for example, consideration of the target indication, the trial endpoints, progression of the disease, disorder, or condition during the trial study period, biochemical or pathophysiologic relevance of the candidate therapeutic intervention, and other variables that were not included or anticipated in the initial study design or clinical protocol. Factors to be included in the economic significance in determining additional testing parameters include sample size, accrual rate, number of clinical sites or institutions required, additional or other available medical or therapeutic interventions approved for human use, and additional or other available medical or therapeutic interventions concurrently or anticipated to enter human clinical testing. Further, there may be patients within the treatment categories that present data that fall outside of the average or mean values, or there may be an indication of multiple allelic loci that are involved in the responses to the candidate therapeutic intervention. In these cases, one could propose a prospective clinical trial having an objective to determine the significance of the variable or parameter and its effect on the outcome of the parent Phase II trial. In the case of a pharmacogenetic difference, i.e. a single or multiple allelic difference, a population could be selected based upon the distribution of genotypes. The candidate therapeutic intervention could then be tested in this group of volunteers to test for efficacy or toxicity. The repeat prospective study could be a Phase I limited study in which the subjects would be healthy human volunteers, or a Phase II limited efficacy study in which patients which satisfy the inclusion criteria could be enrolled. In either case, the second, confirmatory trial could then be used to systematically ensure an adequate number of patients with appropriate phenotype is enrolled in a Phase III trial.

A placebo controlled pharmacogenetics clinical trial design will be one in which target allelic variance or variances will be identified and a diagnostic test will be performed to stratify the patients based upon presence, absence, or combination thereof of these variances. In the Phase II or Phase III stage of clinical development, determination of a specific sample size of a prospective trial will be described to include factors such as expected differences between a placebo and treatment on the primary or secondary endpoints and a consideration of the allelic frequencies.

The design of a pharmacogenetics clinical trial will include a description of the allelic variance impact on the observed efficacy between the treatment groups. Using this type of design, the type of genetic and phenotypic relationship display of the efficacy response to a candidate therapeutic intervention will be analyzed. For example, a genotypically dominant allelic variance or variances will be those in which both heterozygotes and homozygotes will demonstrate a specific phenotypic efficacy response different from the homozygous recessive genotypic group. A pharmacogenetic approach is useful for clinicians and public health professionals to include or eliminate small groups of responders or non-responders from treatment in order to avoid unjustified side-effects. Further, adjustment of dosages when clear clinical difference between heterozygous and homozygous individuals may be beneficial for therapy with the candidate therapeutic intervention

In another example, a recessive allelic variance or variances will be those in which only the homozygote recessive for that or those variances will demonstrate a specific phenotypic efficacy response different from the heterozygotes or homozygous dominants. An extension of these examples may include allelic variance or variances organized by haplotypes from additional gene or genes.

V. Variance Identification and Use

A. Initial Identification of Variances in Genes

Selection of Population Size and Composition

Prior to testing to identify the presence of sequence variances in a particular gene or genes, it is useful to understand how many individuals should be screened to provide confidence that most or nearly all pharmacogenetically relevant variances will be found. The answer depends on the frequencies of the phenotypes of interest and what assumptions we make about heterogeneity and magnitude of genetic effects. Prior to testing to identify the presence of sequence variances in a particular gene or genes, it is useful to understand how many individuals should be screened to provide confidence that most or nearly all pharmacogenetically relevant variances will be found. The answer depends on the frequencies of the phenotypes of interest and what assumptions we make about heterogeneity and magnitude of genetic effects. At the beginning we only know phenotype frequencies (e.g. responders vs. nonresponders, frequency of various side effects, etc.).

The most conservative assumption (resulting in the lowest estimate of allele frequency, and consequently the largest suggested screening population) is (i) that the phenotype (e.g. toxicity or efficacy) is multifactorial (i.e. can be caused by two or more variances or combinations of variances), (ii) that the variance of interest has a high degree of penetrance (i.e. is consistently associated with the phenotype), and (iii) that the mode of transmission is Mendelian dominant. Consider a pharmacogenetic study designed to identify predictors of efficacy for a compound that produces a 15% response rate in a nonstratified population. If half the response is substantially attributable to a given variance, and the variance is consistently associated with a positive response (in 80% of cases) and the variance need only be present in one copy to produce a positive result then ˜10% of the subjects are likely heterozygotes for the variance that produces the response. The Hardy-Weinberg equation can be used to infer an allele frequency in the range of 5% from these assumptions (given allele frequencies of 5%/95% then: 2×0.05×0.95=0.095, or 9.5% heterozygotes are expected, and 0.05×0.05=0.0025, or 0.25% homozygotes are expected. They sum to 9.5%+0.25%=9.75% likely responders, 80% of whom, or 7.6%, are likely real responders due to presence of the positive response allele. Thus about half of the 15% responders are accounted for.). From the Table it can be seen that, in order to have a 99% chance of detecting an allele present at a frequency of 5% nearly 50 subjects should be screened for variances, assuming that the variances occur in the screening population at the same frequency as they occur in the patient population. Similar analyses can be performed for other assumptions regarding likely magnitude of effect, penetrance and mode of genetic transmission.

At the beginning we only know phenotype frequencies (e.g. responders vs. nonresponders, frequency of various side effects, etc.). As an example, the occurrence of serious 5-FU/FA toxicity—e.g. toxicity requiring hospitalization is often >10%. The occurrence of life threatening toxicity is in the 1-3% range (Buroker et al. 1994). The occurrence of complete remissions is on the order of 2-8%. The lowest frequency phenotypes are thus on the order of ˜2%. If we assume that (i) homogeneous genetic effects are responsible for half the phenotypes of interest and (ii) for the most part the extreme phenotypes represent recessive genotypes, then we need to detect alleles that will be present at ˜10% frequency (0.1×0.1=0.01, or 1% frequency of homozygotes) if the population is at Hardy-Weinberg equilibrium. To have a ˜99% chance of identifying such alleles would require searching a population of 22 individuals (see Table below). If the major phenotypes are associated with heterozygous genotypes then we need to detect alleles present at ˜0.5% frequency (2×0.005×0.995=0.00995, or ˜1% frequency of heterozygotes). A 99% chance of detecting such alleles would require ˜40 individuals (Table below). Given the heterogeneity of the North American population we cannot assume that all genotypes are present in Hardy-Weinberg proportions, therefore a substantial oversampling may be done to increase the chances of detecting relevant variances: For our initial screening, usually, 62 individuals of known race/ethnicity are screened for variance. Variance detection studies can be extended to outliers for the phenotypes of interest to cover the possibility that important variances were missed in the normal population screening.

| Allele | | | | | | | | |
|---|---|---|---|---|---|---|---|---|
| frequencies | n = 5 | n = 10 | n = 15 | n = 20 | n = 25 | n = 30 | n = 35 | n = 50 |
| p = .99, | 9.56 | 18.21 | 26.03 | 33.10 | 39.50 | 45.28 | 50.52 | 63.40 |
| p = .97, | 26.26 | 45.62 | 59.90 | 70.43 | 78.19 | 83.92 | 88.14 | 95.24 |
| p = .95, | 40.13 | 64.15 | 78.53 | 87.15 | 92.30 | 95.39 | 97.24 | 99.65 |
| p = .93, | 51.60 | 76.58 | 88.66 | 94.51 | 97.34 | 98.71 | 99.38 | 99.93 |
| p = .9, q = | 65.13 | 87.84 | 95.76 | 98.52 | 99.48 | 99.82 | 99.94 | >99.9 |
| p = .8, q = | 89.26 | 98.84 | 99.88 | 99.99 | >99.9 | >99.9 | >99.9 | >99.9 |
| p = .7, q = | 97.17 | 99.92 | 99.99 | >99.9 | >99.9 | >99.9 | >99.9 | >99.9 |

Likelihood of Detecting Polymorphism in a Population as a Function of Allele Frequency & Number of Individuals Genotyped

The table above shows the probability (expressed as percent) of detecting both alleles (i.e. detecting heterozygotes) at a biallelic locus as a function of (i) the allele frequencies and (ii) the number of individuals genotyped. The chances of detecting heterozygotes increases as the frequencies of the two alleles approach 0.5 (down a column), and as the number of individuals genotyped increases (to the right along a row). The numbers in the table are given by the formula: 1−(p)²ⁿ−(q)²ⁿ. Allele frequencies are designated p and q and the number of individuals tested is designated n. (Since humans are diploid, the number of alleles tested is twice the number of individuals, or 2n.)

While it is preferable that numbers of individuals, or independent sequence samples, are screened to identify variances in a gene, it is also very beneficial to identify variances using smaller numbers of individuals or sequence samples. For example, even a comparison between the sequences of two samples or individuals can reveal sequence variances between them. Preferably, 5, 10, or more samples or individuals are screened.

Source of Nucleic Acid Samples

Nucleic acid samples, for example for use in variance identification, can be obtained from a variety of sources as known to those skilled in the art, or can be obtained from genomic or cDNA sources by known methods. For example, the Coriell Cell Repository (Camden, N.J.) maintains over 6,000 human cell cultures, mostly fibroblast and lymphoblast cell lines comprising the NIGMS Human Genetic Mutant Cell Repository. A catalog (http://locus.umdnj.edu/nigms) provides racial or ethnic identifiers for many of the cell lines. It is preferable to perform polymorphism discovery on a population that mimics the population to be evaluated in a clinical trial, both in terms of racial/ethnic/geographic background and in terms of disease status. Otherwise, it is generally preferable to include a broad population sample including, for example, (for trials in the United States): Caucasians of Northern, Central and Southern European origin, Africans or African-Americans, Hispanics or Mexicans, Chinese, Japanese, American Indian, East Indian, Arabs and Koreans.

Source of Human DNA, RNA and cDNA Samples

PCR based screening for DNA polymorphism can be carried out using either genomic DNA or cDNA produced from mRNA. For many genes, only cDNA sequences have been published, therefore the analysis of those genes is, at least initially, at the cDNA level since the determination of intron-exon boundaries and the isolation of flanking sequences is a laborious process. However, screening genomic DNA has the advantage that variances can be identified in promoter, intron and flanking regions. Such variances may be biologically relevant. Therefore preferably, when variance analysis of patients with outlier responses is performed, analysis of selected loci at the genomic level is also performed. Such analysis would be contingent on the availability of a genomic sequence or intron-exon boundary sequences, and would also depend on the anticipated biological importance of the gene in connection with the particular response.

When cDNA is to be analyzed it is very beneficial to establish a tissue source in which the genes of interest are expressed at sufficient levels that cDNA can be readily produced by RT-PCR. Preliminary PCR optimization efforts for 19 of the 29 genes in Table 2 reveal that all 19 can be amplified from lymphoblastoid cell mRNA. The 7 untested genes belong on the same pathways and are expected to also be PCR amplifiable.

PCR Optimization

Primers for amplifying a particular sequence can be designed by methods known to those skilled in the art, including by the use of computer programs such as the PRIMER software available from Whitehead Institute/MIT Genome Center. In some cases it is preferable to optimize the amplification process according to parameters and methods known to those skilled in the art; optimization of PCR reactions based on a limited array of temperature, buffer and primer concentration conditions is utilized. New primers are obtained if optimization fails with a particular primer set.

Variance Detection Using T4 Endonuclease VII Mismatch Cleavage Method

Any of a variety of different methods for detecting variances in a particular gene can be utilized, such as those described in the patents and applications cited in section A above. An exemplary method is a T4 EndoVII method. The enzyme T4 endonuclease VII (T4E7) is derived from the bacteriophage T4. T4E7 specifically cleaves heteroduplex DNA containing single base mismatches, deletions or insertions. The site of cleavage is 1 to 6 nucleotides 3′ of the mismatch. This activity has been exploited to develop a general method for detecting DNA sequence variances (Youil et al. 1995; Mashal and Sklar, 1995). A quality controlled T4E7 variance detection procedure based on the T4E7 patent of R. G. H. Cotton and co-workers. (Del Tito et al., in press) is preferably utilized. T4E7 has the advantages of being rapid, inexpensive, sensitive and selective. Further, since the enzyme pinpoints the site of sequence variation, sequencing effort can be confined to a 25-30 nucleotide segment.

The major steps in identifying sequence variations in candidate genes using T4E7 are: (1) PCR amplify 400-600 bp segments from a panel of DNA samples; (2) mix a fluorescently-labeled probe DNA with the sample DNA; (3) heat and cool the samples to allow the formation of heteroduplexes; (4) add T4E7 enzyme to the samples and incubate for 30 minutes at 37° C., during which cleavage occurs at sequence variance mismatches; (5) run the samples on an ABI 377 sequencing apparatus to identify cleavage bands, which indicate the presence and location of variances in the sequence; (6) a subset of PCR fragments showing cleavage are sequenced to identify the exact location and identity of each variance.

The T4E7 Variance Imaging procedure has been used to screen particular genes. The efficiency of the T4E7 enzyme to recognize and cleave at all mismatches has been tested and reported in the literature. One group reported detection of 81 of 81 known mutations (Youil et al. 1995) while another group reported detection of 16 of 17 known mutations (Mashal and Sklar, 1995). Thus, the T4E7 method provides highly efficient variance detection.

DNA Sequencing

A subset of the samples containing each unique T4E7 cleavage site is selected for sequencing. DNA sequencing can, for example, be performed on ABI 377 automated DNA sequencers using BigDye chemistry and cycle sequencing. Analysis of the sequencing runs will be limited to the 30-40 bases pinpointed by the T4E7 procedure as containing the variance. This provides the rapid identification of the altered base or bases.

In some cases, the presence of variances can be inferred from published articles which describe Restriction Fragment Length Polymorphisms (RFLP). The sequence variances or polymorphisms creating those RFLPs can be readily determined using convention techniques, for example in the following manner. If the RFLP was initially discovered by the hybridization of a cDNA, then the molecular sequence of the RFLP can be determined by restricting the cDNA probe into fragments and separately hybridizing to a Southern blot consisting of the restriction digestion with the enzyme which reveals the polymorphic site, identifying the sub-fragment which hybridizes to the polymorphic restriction fragment, obtaining a genomic clone of the gene (e.g., from commercial services such as Genome Systems (Saint Louis, Mo.) or Research Genetics (Ala. ) which will provide appropriate genomic clones on receipt of appropriate primer pairs). Using the genomic clone, restrict the genomic clone with the restriction enzyme which revealed the polymorphism and isolate the fragment which contains the polymorphism, e.g., identifying by hybridization to the cDNA which detected the polymorphism. The fragment is then sequenced across the polymorphic site. A copy of the other allele can be obtained by PCT from addition samples.

Variance Detection Using Sequence Scanning

In addition to the physical methods, e.g., those described above and others known to those skilled in the art (see, e.g., Housman, U.S. Pat. No. 5,702,890;

Housman et al., U.S. patent application Ser. No. 09/045,053), variances can be detected using computational methods, involving computer comparison of sequences from two or more different biological sources, which can be obtained in various ways, for example from public sequence databases. The term “variance scanning” refers to a process of identifying sequence variances using computer-based comparison and analysis of multiple representations of at least a portion of one or more genes. Computational variance detection involves a process to distinguish true variances from sequencing errors or other artifacts, and thus does not require perfectly accurate sequences. Such scanning can be performed in a variety of ways, preferably, for example, as described in Stanton et al., filed Oct. 14, 1999, Ser. No. 09/419,705.

While the utilization of complete cDNA sequences is highly preferred, it is also possible to utilize genomic sequences. Such analysis may be desired where the detection of variances in or near splice sites is sought. Such sequences may represent full or partial genomic DNA sequences for a gene or genes. Also, as previously indicated, partial cDNA sequences can also be utilized although this is less preferred. As described below, the variance scanning analysis can simply utilize sequence overlap regions, even from partial sequences. Also, while the present description is provided by reference to DNA, e.g., cDNA, some sequences may be provided as RNA sequences, e.g., mRNA sequences. Such RNA sequences may be converted to the corresponding DNA sequences, or the analysis may use the RNA sequences directly.

B. Determination of Presence or Absence of Known Variances

The identification of the presence of previously identified variances in cells of an individual, usually a particular patient, can be performed by a number of different techniques as indicated in the Summary above. Such methods include methods utilizing a probe which specifically recognizes the presence of a particular nucleic acid or amino acid sequence in a sample. Common types of probes include nucleic acid hybridization probes and antibodies, for example, monoclonal antibodies, which can differentially bind to nucleic acid sequences differing in one or more variance sites or to polypeptides which differ in one or more amino acid residues as a result of the nucleic acid sequence variance or variances. Generation and use of such probes is well-known in the art and so is not described in detail herein.

Preferably, however, the presence or absence of a variance is determined using nucleotide sequencing of a short sequence spanning a previously identified variance site. This will utilize validated genotyping assays for the polymorphisms previously identified. Since both normal and tumor cell genotypes can be measured, and since tumor material will frequently only be available as paraffin embedded sections (from which RNA cannot be isolated), it will be necessary to utilize genotyping assays that will work on genomic DNA. Thus PCR reactions will be designed, optimized, and validated to accommodate the intron-exon structure of each of the genes. If the gene structure has been published (as it has for some of the listed genes), PCR primers can be designed directly. However, if the gene structure is unknown, the PCR primers may need to be moved around in order to both span the variance and avoid exon-intron boundaries. In some cases one-sided PCR methods such as bubble PCR (Ausubel et al. 1997) may be useful to obtain flanking intronic DNA for sequence analysis.

Using such amplification procedures, the standard method used to genotype normal and tumor tissues will be DNA sequencing. PCR fragments encompassing the variances will be cycle sequenced on ABI 377 automated sequencers using Big Dye chemistry

C. Correlation of the Presence or Absence of Specific Variances with Differential Treatment Response

Prior to establishment of a diagnostic test for use in the selection of a treatment method or elimination of a treatment method, the presence or absence of one or more specific variances in a gene or in multiple genes is correlated with a differential treatment response. (As discussed above, usually the existence of a variable response and the correlation of such a response to a particular gene is performed first.) Such a differential response can be determined using prospective and/or retrospective data. Thus, in some cases, published reports will indicate that the course of treatment will vary depending on the presence or absence of particular variances. That information can be utilized to create a diagnostic test and/or incorporated in a treatment method as an efficacy or safety determination step.

Usually, however, the effect of one or more variances is separately determined. The determination can be performed by analyzing the presence or absence of particular variances in patients who have previously been treated with a particular treatment method, and correlating the variance presence or absence with the observed course, outcome, and/or development of adverse events in those patients. This approach is useful in cases in which observation of treatment effects was clearly recorded and cell samples are available or can be obtained. Alternatively, the analysis can be performed prospectively, where the presence or absence of the variance or variances in an individual is determined and the course, outcome, and/or development of adverse events in those patients is subsequently or concurrently observed and then correlated with the variance determination.

Analysis of Haplotypes Increases Power of Genetic Analysis

In some cases, variation in activity due to a single gene or a single genetic variance in a single gene may not be sufficient to account for a clinically significant fraction of the observed variation in patient response to a treatment, e.g., a drug, there may be other factors that account for some of the variation in patient response. Drug response phenotypes may vary continuously, and such (quantitative) traits may be influenced by a number of genes (Falconer and Mackay, Quantitative Genetics, 1997). Although it is impossible to determine a priori the number of genes influencing a quantitative trait, potentially only one or a few loci have large effects, where a large effect is 5-20% of total variation in the phenotype (Mackay, 1995).

Having identified genetic variation in enzymes that may affect action of a specific drug, it is useful to efficiently address its relation to phenotypic variation. The sequential testing for correlation between phenotypes of interest and single nucleotide polymorphisms may be adequate to detect associations if there are major effects associated with single nucleotide changes; certainly it is useful to this type of analysis. However there is no way to know in advance whether there are major phenotypic effects associated with single nucleotide changes and, even if there are, there is no way to be sure that the salient variance has been identified by screening cDNAs. A more powerful way to address the question of genotype-phenotype correlation is to assort genotypes into haplotypes. (A haplotype is the cis arrangement of polymorphic nucleotides on a particular chromosome.) Haplotype analysis has several advantages compared to the serial analysis of individual polymorphisms at a locus with multiple polymorphic sites.

(1) Of all the possible haplotypes at a locus (2ⁿ haplotypes are theoretically possible at a locus with n binary polymorphic sites) only a small fraction will generally occur at a significant frequency in human populations. Thus, association studies of haplotypes and phenotypes will involve testing fewer hypotheses. As a result there is a smaller probability of Type I errors, that is, false inferences that a particular variant is associated with a given phenotype.

(2) The biological effect of each variance at a locus may be different both in magnitude and direction. For example, a polymorphism in the 5′ UTR may affect translational efficiency, a coding sequence polymorphism may affect protein activity, a polymorphism in the 3′ UTR may affect mRNA folding and half life, and so on. Further, there may be interactions between variances: two neighboring polymorphic amino acids in the same domain—say cys/arg at residue 29 and met/val at residue 166—may, when combined in one sequence, for example, 29 cys-166 val, have a deleterious effect, whereas 29 cys-166 met, 29 arg-166 met and 29 arg-166 val proteins may be nearly equal in activity. Haplotype analysis is the best method for assessing the interaction of variances at a locus.

(3) Templeton and colleagues have developed powerful methods for assorting haplotypes and analyzing haplotype/phenotype associations (Templeton et al., 1987). Alleles which share common ancestry are arranged into a tree structure (cladogram) according to their (inferred) time of origin in a population (that is, according to the principle of parsimony). Haplotypes that are evolutionarily ancient will be at the center of the branching structure and new ones (reflecting recent mutations) will be represented at the periphery, with the links representing intermediate steps in evolution. The cladogram defines which haplotype-phenotype association tests should be performed to most efficiently exploit the available degrees of freedom, focusing attention on those comparisons most likely to define functionally different haplotypes (Haviland et al., 1995). This type of analysis has been used to define interactions between heart disease and the apolipoprotein gene cluster (Haviland et al 1995) and Alzheimer's Disease and the Apo-E locus (Templeton 1995) among other studies, using populations as small as 50 to 100 individuals. The methods of Templeton have also been applied to measure the genetic determinants of variation in the angiotensin-I converting enzyme gene. (Keavney, B., McKenzie, C. A., Connoll, J. M. C., et al. Measured haplotype analysis of the angiotensin-I converting enzyme gene. Human Molecular Genetics 7: 1745-1751.)

Methods for Determining Haplotypes

The goal of haplotyping is to identify the common haplotypes at selected loci that have multiple sites of variance. Haplotypes are usually determined at the cDNA level. Several general approaches to identification of haplotyes can be employed. Haplotypes may also be estimated using computational methods or determined definitively using experimental approaches. Computational approaches generally include an expectation maximization (E-M) algorithm (see, for example: Excoffier and Slatkin, Mol. Biol. Evol. 1995) or a combination of Parsimony (see below) and E-M methods.

Haplotypes can be determined experimentally without requirement of a haplotyping method by genotyping samples from a set of pedigrees and observing the segregation of haplotypes. For example families collected by the Centre d'Etude du Polymorphisme Humaine (CEPH) can be used. Cell lines from these families are available from the Coriell Repository. This approach will be useful for cataloging common haplotypes and for validating methods on samples with known haplotypes. The set of haplotypes determined by pedigree analysis can be useful in computational methods, including those utilizing the E-M algorithm.

Haplotypes can also be determined directly from cDNA using the T4E7 procedure. T4E7 cleaves mismatched heteroduplex DNA at the site of the mismatch. If a heteroduplex contains only one mismatch, cleavage will result in the generation of two fragments. However, if a single heteroduplex (allele) contains two mismatches, cleavage will occur at two different sites resulting in the generation of three fragments. The appearance of a fragment whose size corresponds to the distance between the two cleavage sites is diagnostic of the two mismatches being present on the same strand (allele). Thus, T4E7 can be used to determine haplotypes in diploid cells.

An alternative method, allele specific PCR, may be used for haplotyping. The utility of allele specific PCR for haplotyping has already been established (Michalatos-Beloin et al., 1996; Chang et al. 1997). Opposing PCR primers are designed to cover two sites of variance (either adjacent sites or sites spanning one or more internal variances). Two versions of each primer are synthesized, identical to each other except for the 3′ terminal nucleotide. The 3′ terminal nucleotide is designed so that it will hybridize to one but not the other variant base. PCR amplification is then attempted with all four possible primer combinations in separate wells. Because Taq polymerase is very inefficient at extending 3′ mismatches, the only samples which will be amplified will be the ones in which the two primers are perfectly matched for sequences on the same strand (allele). The presence or absence of PCR product allows haplotyping of diploid cell lines. At most two of four possible reactions should yield products. This procedure has been successfully applied, for example, to haplotype the DPD amino acid polymorphisms.

Parsimony methods are also useful for classifying DNA sequences, haplotypes or phenotypic characters. Parsimony principle maintains that the best explanation for the observed differences among sequences, phenotypes (individuals, species) etc., is provided by the smallest number of evolutionary changes. Alternatively, simpler hypotheses are preferable to explain a set of data or patterns, than more complicated ones, and ad hoc hypotheses should be avoided whenever possible (Molecular Systematics, Hillis et al., 1996). Parsimony methods thus operate by minimizing the number of evolutionary steps or mutations (changes from one sequence/character) required to account for a given set of data.

For example, supposing we want to obtain relationships among a set of sequences and construct a structure (tree/topology), we first count the minimum number of mutations that are required for explaining the observed evolutionary changes among a set of sequences. A structure (topology) is constructed based on this number. When once this number is obtained, another structure is tried. This process is continued for all reasonable number of structures. Finally, the structure that required the smallest number of mutational steps is chosen as the likely structure/evolutionary tree for the sequences studied.

For haplotypes identified herein, haplotypes were identified by examining genotypes from each cell line. This list of genotypes was optimized to remove variance sites/individuals with incomplete information, and the genotype from each remaining cell line was examined in turn. The number of heterozygotes in the genotype were counted, and those genotypes containing more than one heterozygote were discarded, and the rest were gathered in a list for storage and display. For haplotypes identified herein, haplotypes were identified by examining genotypes from each cell line. This list of genotypes was optimized to remove variance sites/individuals with incomplete information, and the genotype from each remaining cell line was examined in turn. The number of heterozygotes in the genotype were counted, and those genotypes containing more than one heterozygote were discarded, and the rest were gathered in a list for storage and display.

D. Selection of Treatment Method Using Variance Information

1. General

Once the presence or absence of a variance or variances in a gene or genes is shown to correlate with the efficacy or safety of a treatment method, that information can be used to select an appropriate treatment method for a particular patient. In the case of a treatment which is more likely to be effective when administered to a patient who has at least one copy of a gene with a particular variance or variances (in some cases the correlation with effective treatment is for patients who are homozygous for a variance or set of variances in a gene) than in patients with a different variance or set of variances, a method of treatment is selected (and/or a method of administration) which correlates positively with the particular variance presence or absence which provides the indication of effectiveness. As indicated in the Summary, such selection can involve a variety of different choices, and the correlation can involve a variety of different types of treatments, or choices of methods of treatment. In some cases, the selection may include choices between treatments or methods of administration where more than one method is likely to be effective, or where there is a range of expected effectiveness or different expected levels of contra-indication or deleterious effects. In such cases the selection is preferably performed to select a treatment which will be as effective or more effective than other methods, while having a comparatively low level of deleterious effects. Similarly, where the selection is between method with differing levels of deleterious effects, preferably a method is selected which has low such effects but which is expected to be effective in the patient.

Alternatively, in cases where the presence or absence of the particular variance or variances is indicative that a treatment or method of administration is more likely to be ineffective or contra-indicated in a patient with that variance or variances, then such treatment or method of administration is generally eliminated for use in that patient.

2. Diagnostic Methods

Once a correlation between the presence and absence of at least one variance in a gene or genes and an indication of the effectiveness of a treatment, the determination of the presence or absence of that at least one variance provides diagnostic methods, which can be used as indicated in the Summary above to select methods of treatment, methods of administration of a treatment, methods of selecting a patient or patients for a treatment and others aspects in which the determination of the presence or absence of those variances provides useful information for selecting or designing or preparing methods or materials for medical use in the aspects of this invention. As previously stated, such variance determination or diagnostic methods can be performed in various ways as understood by those skilled in the art.

In certain variance determination methods, it is necessary or advantageous to amplify one or more nucleotide sequences in one or more of the genes identified herein. Such amplification can be performed by conventional methods, e.g., using polymerase chain reaction (PCR) amplification. Such amplification methods are well-known to those skilled in the art and will not be specifically described herein. For most applications relevant to the present invention, a sequence to be amplified includes at least one variance site, which is preferably a site or sites which provide variance information indicative of the effectiveness of a method of treatment or method of administration of a treatment, or effectiveness of a second method of treatment which reduces a deleterious effect of a first treatment method, or which enhances the effectiveness of a first method of treatment. Thus, for PCR, such amplification generally utilizes primer oligonucleotides which bind to or extent through at least one such variance site under amplification conditions.

For convenient use of the amplified sequence, e.g., for sequencing, it is beneficial that the amplified sequence be of limited length, but still long enough to allow convenient and specific amplification. Thus, preferably the amplified sequence has a length as described in the Summary.

Also, in certain variance determination, it is useful to sequence one or more portions of a gene or genes, in particular, portions of the genes identified in this disclosure. As understood by persons familiar with nucleic acid sequencing, there are a variety of effective methods. In particular, sequencing can utilize dye termination methods and mass spectrometric methods. The sequencing generally involves a nucleic acid sequence which includes a variance site as indicated above in connection with amplification. Such sequencing can directly provide determination of the presence or absence of a particular variance or set of variances, e.g., a haplotype, by inspection of the sequence (visually or by computer). Such sequencing is generally conducted on PCR amplified sequences in order to provide sufficient signal for practical or reliable sequence determination.

Likewise, in certain variance determinations, it is useful to utilize a probe or probes. As previously described, such probes can be of a variety of different types.

VI. Pharmaceutical Compositions, Including Pharmaceutical Compositions Adapted to be Preferentially Effective in Patients Having Particular Genetic Characteristics

A. General

The methods of the present invention, in many cases will utilize conventional pharmaceutical compositions, but will allow more advantageous and beneficial use of those compositions due to the ability to identify patients who are likely to benefit from a particular treatment or to identify patients for whom a particular treatment is less likely to be effective or for whom a particular treatment is likely to produce undesirable or intolerable effects. However, in some cases, it is advantageous to utilize compositions which are adapted to be preferentially effective in patients who possess particular genetic characteristics, i.e., in whom a particular variance or variances in one or more genes is present or absent (depending on whether the presence or the absence of the variance or variances in a patient is correlated with an increased expectation of beneficial response). Thus, for example, the presence of a particular variance or variances may indicate that a patient can beneficially receive a significantly higher dosage of a drug than a patient having a different

B. Regulatory Indications and Restrictions

The sale and use of drugs and the use of other treatment methods usually are subject to certain restrictions by a government regulatory agency charged with ensuring the safety and efficacy of drugs and treatment methods for medical use, and approval is based on particular indications. In the present invention it is found that variability in patient response or patient tolerance of a drug or other treatment often correlates with the presence or absence of particular variances in particular genes. Thus, it is expected that such a regulatory agency may indicate that the approved indications for use of a drug with a variance-related variable response or toleration include use only in patients in whom the drug will be effective, and/or for whom the administration of the drug will not have intolerable deleterious effects, such as excessive toxicity or unacceptable side-effects. Conversely, the drug may be given for an indication that it may be used in the treatment of a particular disease or condition where the patient has at least one copy of a particular variance, variances, or variant form of a gene. Even if the approved indications are not narrowed to such groups, the regulatory agency may suggest use limited to particular groups or excluding particular groups or may state advantages of use or exclusion of such groups or may state a warning on the use of the drug in certain groups. Consistent with such suggestions and indications, such an agency may suggest or recommend the use of a diagnostic test to identify the presence or absence of the relevant variances in the prospective patient. Such diagnostic methods are described in this description. Generally, such regulatory suggestion or indication is provided in a product insert or label, and is generally reproduced in references such as the Physician's Desk Reference (PDR). Thus, this invention also includes drugs or pharmaceutical compositions which carry such a suggestion or statement of indication or warning or suggestion for a diagnostic test, and which may also be packaged with an insert or label stating the suggestion or indication or warning or suggestion for a diagnostic test.

In accord with the possible variable treatment responses, an indication or suggestion can specify that a patient be heterozygous, or alternatively, homozygous for a particular variance or variances or variant form of a gene. Alternatively, an indication or suggestion may specify that a patient have no more than one copy, or zero copies, of a particular variance, variances, or variant form of a gene.

A regulatory indication or suggestion may concern the variances or variant forms of a gene in normal cells of a patient and/or in cells involved in the disease or condition. For example, in the case of a cancer treatment, the response of the cancer cells can depend on the form of a gene remaining in cancer cells following loss of heterozygosity affecting that gene. Thus, even though normal cells of the patient may contain a form of the gene which correlates with effective treatment response, the absence of that form in cancer cells will mean that the treatment would be less likely to be effective in that patient than in another patient who retained in cancer cells the form of the gene which correlated with effective treatment response. Those skilled in the art will understand whether the variances or gene forms in normal or disease cells are most indicative of the expected treatment response, and will generally utilize a diagnostic test with respect to the appropriate cells. Such a cell type indication or suggestion may also be contained in a regulatory statement, e.g., on a label or in a product insert.

C. Preparation and Administration of Drugs and Pharmaceutical Compositions Including Pharmaceutical Compositions Adapted to be Preferentially Effective in Patients Having Particular Genetic Characteristics

A particular compound useful in this invention can be administered to a patient either by itself, or in pharmaceutical compositions where it is mixed with suitable carriers or excipient(s). In treating a patient exhibiting a disorder of interest, a therapeutically effective amount of a agent or agents such as these is administered. A therapeutically effective dose refers to that amount of the compound that results in amelioration of one or more symptoms or a prolongation of survival in a patient.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized.

For any compound used in the method of the invention, the therapeutically effective dose can be estimated initially from cell culture assays. For example, a dose can be formulated in animal models to achieve a circulating plasma concentration range that includes the IC₅₀ as determined in cell culture. Such information can be used to more accurately determine useful doses in humans. Levels in plasma may be measured, for example, by HPLC.

The exact formulation, route of administration and dosage can be chosen by the individual physician in view of the patient's condition. (See e.g. Fingl et. al., in The Pharmacological Basis of Therapeutics, 1975, Ch. 1 p.1). It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administrated dose in the management of disorder of interest will vary with the severity of the condition to be treated and the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Depending on the specific conditions being treated, such agents may be formulated and administered systemically or locally. Techniques for formulation and administration may be found in Remington's Pharmaceutical Sciences, 18th ed., Mack Publishing Co., Easton, Pa. (1990). Suitable routes may include oral, rectal, transdermal, vaginal, transmucosal, or intestinal administration; parenteral delivery, including intramuscular, subcutaneous, intramedullary injections, as well as intrathecal, direct intraventricular, intravenous, intraperitoneal, intranasal, or intraocular injections, just to name a few.

For injection, the agents of the invention may be formulated in aqueous solutions, preferably in physiologically compatible buffers such as Hanks's solution, Ringer's solution, or physiological saline buffer. For such transmucosal administration, penetrants appropriate to the barrier to be permeated are used in the formulation. Such penetrants are generally known in the art.

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Agents intended to be administered intracellularly may be administered using techniques well known to those of ordinary skill in the art. For example, such agents may be encapsulated into liposomes, then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm. Additionally, due to their hydrophobicity, small organic molecules may be directly administered intracellularly.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions. The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

The invention described herein features methods for determining the appropriate identification of a patient diagnosed with a neurological disease or neurological dysfunction based on an analysis of the patient's allele status for a gene listed in Tables 1, 3, and 4. Specifically, the presence of at least one allele indicates that a patient will respond to a candidate therapeutic intervention aimed at treating a neurological clinical symptoms. In a preferred approach, the patient's allele status is rapidly diagnosed using a sensitive PCR assay and a treatment protocol is rendered. The invention also provides a method for forecasting patient outcome and the suitability of the patient for entering a clinical drug trial for the testing of a candidate therapeutic intervention for a neurological disease, condition, or dysfunction.

The findings described herein indicate the predictive value of the target allele in identifying patients at risk for neurologic disease or neurologic dysfunction. In addition, because the underlying mechanism influenced by the allele status is not disease-specific, the allele status is suitable for making patient predictions for diseases not affected by the pathway as well.

The following examples, which describe exemplary techniques and experimental results, are provided for the purpose of illustrating the invention, and should not be construed as limiting.

### EXAMPLE 1

### Effect of Pharmacokinetic parameters on Efficacy of Drugs and Candidate Therapeutic Interventions

The efficacy of a compound is determined by a combination of pharmacodynamic and pharmacokinetic effects. Both types of effect are under genetic control. In the present invention, the genetic determinants of efficacy are discussed in terms of variation in the genes that encode proteins responsible for absorption, distribution, metabolism, and excretion of compounds, i.e. pharmacokinetic parameters.

The pharmacokinetic parameters with potential effects on efficacy include absorption, distribution, metabolism, and excretion. These parameters affect efficacy broadly by controlling the availability of a compound at the site(s) of action. Interpatient variability in the availability of a compound can result in undertreatment or overtreatment, or in adverse reactions due to levels of a compound or its metabolite(s). Differences in the genes responsible for pharmacokinetic variation, therefore, can be a potential source of interpatient variability in drug response.

Impact of Stratification Based Upon Genotype in Drug Development for Drugs, Compounds, or Candidate Therapeutic Interventions that may Efficacy

Clozapine induced agranulocytosis has been associated in some reports with specific HLA haplotypes or with HSP70 variants. These reports suggest that a gene within the HLA region is associated with agranulocytosis in response to clozapine therapy. In a recent study, two ethnic groups were analyzed for genetic markers for agranulocytosis. Tumor necrosis factor microsatellites d3 and b4 were found in higher frequencies in patients that experience clozapine-induced agranulocytosis. These data, while they need to be confirmed by additional studies, are suggestive that tumor necrosis factor polymorphisms may also be associated with clozapine-induced agranulocytosis.

In this invention we provide additional genes and gene sequence variances that may account for variability in toxic responses. The Detailed Description above demonstrates how identification of a candidate gene or genes (e.g. gene pathways), genetic stratification, clinical trial design, and diagnostic genotyping can lead to improved medical management of a disease and/or approval of a drug, or broader use of an already approved drug. Gene pathways including, but not limited to, those that are outlined in the gene pathway, Table 1, are useful in identifying the sources of interpatient variation in efficacy as well as in the adverse events summarized in the column headings of Table 2, Discussed in detail below are exemplary candidate genes for the analysis of pharmacokinetic variability in clinical development, using the methods described above.

Advantages of Inclusion of Pharmacogenetic Stratification in Clinical Development of Agents: Impact on Efficacy

The advantages of a clinical research and drug development program that includes the use of polymorphic genotyping for the stratification of patients for the appropriate selection of candidate therapeutic intervention includes 1) identification of patients that may respond earlier and show signs and symptoms of efficacious therapy, 2) identification of the primary gene and relevant polymorphic variance that directly affects efficacy endpoints, 3) identification of pathophysiologic relevant variance or variances and potential therapies affecting those allelic genotypes or haplotypes, and 4) identification of allelic variances or haplotypes in genes that indirectly affects efficacy, safety or both.

By identifying subsets of patients, based upon genotype, that experience efficacious therapeutic benefit in response to the administration of a drug, agent or candidate therapeutic intervention, optimal selection may reduce level and extent of the appearance or manifestation of a side effect or toxicity. Appropriate genotyping and correlation to dosing regimen, or selection of optimal therapy would be beneficial to the patient, caregivers, medical personnel, and the patient's loved ones.

As an example of identification of the primary gene and relevant polymorphic variance that directly affects efficacy, safety, or both one could select an gene pathway as described in the Detailed Description, and determine the effect of genetic polymorphism and therapy efficacy, safety, or both within that given pathway. For example, referring to Table 2, genes involved in absorption and distribution, phase I and phase II metabolism, and excretion the optimization of therapy of by an agent known to have an efficacious effect by determining whether the patient has a predisposing genotype in which the selected agents are more effective and or are more safe. In considering an optimization protocol, one could potentially predetermine the genotypic profile of these genes involved in the manifestation of the adverse effect, or those genes preeminently responsible for drug response. By embarking on the previously described gene pathway approach, it is technical feasibility to determine the relevant genes within such a targeted drug development program.

Identification of pathophysiologic relevant variance or variances and potential therapies affecting those allelic genotypes or haplotypes may speed drug development for therapeutic alternatives. There is a need for therapies that are targeted to a disease and symptom management with limited or no undesirable side effects. Identification of a specific variance or variances within genes involved in the manifestation of clinical efficacious endpoints or therapeutic benefit and specific genetic polymorphisms of these critical genes may assist the development of novel agents and the identification of those patients that may best benefit from therapy of these candidate therapeutic alternatives.

By identifying allelic variances or haplotypes in genes that indirectly affects efficacy, safety of any class of drugs, one could target specific secondary drug or agent therapeutic actions that affect the overall therapeutic action of these agents.

Pharmacogenomics studies for these drugs, or other agent, compound, drug, or candidate therapeutic intervention, could be performed by identifying genes that are involved in the function of a drug including, but not limited to absorption, distribution, metabolism, or elimination, the interaction of the drug with its target as well as potential alternative targets, the response of the cell to the binding of a drug to a target, the metabolism (including synthesis, biodistribution or elimination) of natural compounds which may alter the activity of the drug by complementary, competitive or allosteric mechanisms that potentiate or limit the effect of the drug, and genes involved in the etiology of the disease that alter its response to a particular class of therapeutic agents. It will be recognized to those skilled in the art that this broadly includes proteins involved in pharmacokinetics as well as genes involved in pharmacodynamics. This also includes genes that encode proteins homologous to the proteins believed to carry out the above functions are also worth evaluation as they may carry out similar functions. Together the foregoing proteins constitute the candidate genes for affecting response of a patient to the therapeutic intervention. Using the methods described above, variances in these genes can be identified, and research and clinical studies can be performed to establish an association between a drug response or toxicity and specific variances.

### EXAMPLE 2

### Drug-Induced Toxicity: Blood Dyscrasias

I. Description of Blood Dyscrasias

Blood dyscrasias are a feature of over half of all drug-related deaths and include, but are not limited to, bone marrow aplasia, granulocytopenia, aplastic anemia, leukopenia, lymphoid hyperplasia, hemolytic anemia, and thrombocytopenia. All of these syndromes include pancytopenia to some degree.

Bone marrow aplasia—is defined as a profound loss of bone marrow resulting in pancytopenia. Drugs known to cause bone marrow aplasia include, but are not limited to, chloramphenicol, gold salts, mephenytoin, penicillamine, phenylbutazone, and trimethadione. In general these drugs are not first line therapy due to the rare occurrence of marrow aplasia. Specific forms of aplasia include:

Granulocytopenia—is defined as a loss of polymorphonuclear neutrophils to a count lower than 500. Granulocytopenia primarily predisposes the patient to bacterial and fungal infections. Drugs known to cause granulocytopenia include, but are not limited to, captopril, cephalosporins, choral hydrate, chlorpropamide, penicillins, phenothiazines, phenylbutazone, phenytoin, procainamide, propranolol, and tolbutamide.

Aplastic anemia—is a disorder involving an inability of the hematologic cells to regenerate and thus there is a dramatic depletion of one or more of the following cell types: neutrophils, platelets, or reticulocytes. Drugs associated with producing aplastic anemia are: 1) agents or compounds that produce bone marrow depression, for example cytotoxic drugs used in cancer chemotherapy; 2) agents or compounds that frequently, but inevitably, produce marrow aplasia, for example benzene; 3) agents or compounds that are associated with aplastic anemia, for example chloramphenicol, antiprotozoals, and sulfonamides.

Aplastic anemia is almost always a result of damage to the hematopoietic stem cells. There are two possible routes for the destruction of these cells: 1) direct damage to the stem cell DNA, and 2) cell cycle dependant depletion of later stage progenitor cells. In the first case, drugs or agents bind to and randomly damage the genetic material. This type of aplasia is associated with both early aplasia (immediate or direct cytotoxicity) or later myelodysplasia and leukemia. In the latter case, mitotically and metabolically active progenitor cells are preferentially affected and progenitor cell depletion may lead to unregulated proliferation of spared stem cells.

Leukopenia—is defined when the circulating peripheral white cell count falls below 5-10×10⁹ cells per liter. Circulating leukocytes consist of neutrophils, monocytes, basophils, eosinophils, and lymphocytes.

Neutropenia is defined when the peripheral neutrophil count falls below 2×10⁹ cells per liter. There are a number of drugs families that can cause neutropenia including, but not exclusive to, antiarrythmics (procainamide, propanolol, quinidine), antibiotics (chloramphenicol, penicillins, sulfonamides, trimethorpimmethoxazole, para-aminosalicyclic acid, rifampin, vancomycin, isoniazid, nitrofurantoin), antimalarials (dapsone, qunine, pyrimethamine), anticonvulsants (phenytoin, mephenytoin, trimethadione, ethosuximide, carbamazepine), hypoglycemic agents (tolbutamide, chlorpropamide), antihistamines (cimetadine, brompheniramine, tripelennamine), antihypertensives (methydopa, captopril), antiinflammatory agents (aminopyrine, phenylbutazone, gold salts, ibuprofen, indomethacin), diuretics (acetazolamide, hydrochlorothiazide, chlorthalidone), phenothiazines (chlorpormazine, promazine, prochlorperazine), antimetabolite immunosuppresive agents, cytotoxic agents (alkylating agents, antimetabolites, anthracyclines, vinca alkyloids, cis-platinum, hydroxyarea, actinomycin D), and other agents (alpha and gamma interferon, allopurinol, ethanol, levamisole, penicillamine).

Lymphoid hyperplasia—is characterized by reactive changes within the T-cell regions of the lymph node that encroach on, and at times appear to efface, the germinal follicles. In these regions, the T-cells undergo progressive transformation to immunoblasts. These reactions are encountered particularly in response to drug-induced immunoreactivity. Drugs known to cause lymphoid hyperplasia are phenytoin, and mephenytoin.

Hemolytic anemia—is characterized by the premature destruction of red cells, accumulation of hemoglobin metabolic by-products, and a marked increase in erythroporesis within the bone marrow. Drugs know to cause hemolytic anemia include, but are not excluded to, methyldopa, penicillin, sulfonamides, and vitamin E deficiency.

Thrombocytopenia—is characterized by a marked reduction in the number of circulating platelets to a level below 100,000/mm³. Drug-induced thrombocytopenia may result from decreased production of platelets or decreased platelet survival or both. Drugs known to cause thrombocytopenia include, but are not excluded to, ethanol, acetominophen, acetazolamide, acetylsalicyclic acid, 5-aminosalicylic acid, carbamazepine, chlorpheniramine, cimetadine, digitoxin, diltiazem, ethychlorynol, gold salts, heparin, hydantoins, isoniazid, levodopa, meprobamate, methyldopa, penicillamine, phenylbutazone, procainamide, quinidine, quinine, ranitidine, Rauwolfa alkaloids, rifampin, sulfonamides, sulfonylureas, cytotoxic drugs, and thiazide diuretics.

II. Impact of Stratification Based Upon Genotype in Drug Development for Drugs, Compounds, or Candidate Therapeutic Interventions that may Induce Blood Dyscrasias

Clozapine induced agranulocytosis is associated with differing HLA types and HSP70 variants in patients for whom responded to clozapine therapy but developed agranulocytosis. This is suggestive that a gene within the MHC region is associated with the manifestation of agranulocytosis in response to clozapine therapy. In a recent study, two ethnic groups were analyzed for genetic markers for the agranulocytosis. Tumor necrosis factor microsatellites d3 and b4 were found in higher frequencies in patients that experience clozapine-induced agranulocytosis. These data are suggestive that there is an involvement of tumor necrosis factor constellation polymorphism and clozapine-induced agranulocytosis.

There is evidence to suggest that there are safety response differences to drug therapy in reference to development of blood dyscrasias which may be attributable to genotypic differences between individuals. There is provided in this invention examples of gene pathways that are implicated in the disease process or its therapy and those that potentially cause this variability. The Detailed Description above demonstrates how identification of a candidate gene or genes and gene pathways, stratification, clinical trial design, and implementation of genotyping for appropriate medical management of a given disease can be used to identify the genetic cause of variations in clinical response to therapy, new diagnostic tests, new therapeutic approaches for treating this disorder, and new pharmaceutical products or formulations for therapy. Gene pathways including, but not limited to, those that are outlined in the gene pathway Table 1, and pathway matrix Table 2 and discussed below are candidates for the genetic analysis and product development using the methods described above.

Advantages of Inclusion of Pharmacogenetic Stratification in Clinical Development of Agents that May Cause Blood Dyscrasias

The advantages of a clinical research and drug development program that includes the use of polymorphic genotyping for the stratification of patients for the appropriate selection of candidate therapeutic intervention includes 1) identification of patients that may respond earlier and show signs and symptoms of blood dyscrasias, 2) identification of the primary gene and relevant polymorphic variance that directly affects manifestation of a blood disorder, 3) identification of pathophysiologic relevant variance or variances and potential therapies affecting those allelic genotypes or haplotypes, and 4) identification of allelic variances or haplotypes in genes that indirectly affects efficacy, safety or both.

By identifying subsets of patients, based upon genotype, that experience blood dyscrasias in response to the administration of a drug, agent or candidate therapeutic intervention, optimal selection may reduce level and extent of the hemostatic damage. Appropriate genotyping and correlation to dosing regimen, or selection of optimal therapy would be beneficial to the patient, caregivers, medical personnel, and the patient's loved ones.

As an example of identification of the primary gene and relevant polymorphic variance that directly affects efficacy, safety, or both one could select an gene pathway as described in the Detailed Description, and determine the effect of genetic polymorphism and therapy efficacy, safety, or both within that given pathway. For example, referring to Table 2, genes involved in drug transport, phase I and phase II metabolism, protection from reactive intermediate damage, and immune responsiveness the optimization of therapy of by an agent known to have a blood dyscrasia side effect by determining whether the patient has a predisposing genotype in which the selected agents are more effective and or are more safe. In considering an optimization protocol, one could potentially predetermine the genotypic profile of these genes involved in the manifestation of the adverse effect, or those genes preeminently responsible for drug response. By embarking on the previously described gene pathway approach, it is technical feasibility to determine the relevant genes within such a targeted drug development program.

Identification of pathophysiologic relevant variance or variances and potential therapies affecting those allelic genotypes or haplotypes may speed drug development for therapeutic alternatives. There is a need for therapies that are targeted to a disease and symptom management with limited or no undesirable side effects. Identification of a specific variance or variances within genes involved in the pathophysiologic manifestation of blood dyscrasisas and specific genetic polymorphisms of these critical genes may assist the development of novel agents and the identification of those patients that may best benefit from therapy of these candidate therapeutic alternatives.

By identifying allelic variances or haplotypes in genes that indirectly affects efficacy, safety of any class of drugs that has an effect on the prevention, progression, or symptoms of blood dyscrasias, one could target specific secondary drug or agent therapeutic actions that affect the overall therapeutic action of hemoprotective agents.

Pharmacogenomics studies for these drugs, or other agent, compound, drug, or candidate therapeutic intervention, could be performed by identifying genes that are involved in the function of a drug including, but not limited to absorption, distribution, metabolism, or elimination, the interaction of the drug with its target as well as potential alternative targets, the response of the cell to the binding of a drug to a target, the metabolism (including synthesis, biodistribution or elimination) of natural compounds which may alter the activity of the drug by complementary, competitive or allosteric mechanisms that potentiate or limit the effect of the drug, and genes involved in the etiology of the disease that alter its response to a particular class of therapeutic agents. It will be recognized to those skilled in the art that this broadly includes proteins involved in pharmacokinetics as well as genes involved in pharmacodynamics. This also includes genes that encode proteins homologous to the proteins believed to carry out the above functions are also worth evaluation as they may carry out similar functions. Together the foregoing proteins constitute the candidate genes for affecting response of a patient to the therapeutic intervention. Using the methods described above, variances in these genes can be identified, and research and clinical studies can be performed to establish an association between a drug response or toxicity and specific variances.

### EXAMPLE 3

### Drug-Induced Toxicity: Cutaneous Toxicity

Drug-induced cutaneous toxicity includes, but is not excluded to, eczematous: photodermititis (phototoxic and photoallergic), exfoliative dermititis; maculopapular eruption; papulosquamous reactions: psoriaform, lichus planus, or pityriasis rosea-like; vesiculobullous reactions; toxic epidermal necrolysis; pustular-acneform reactions; urticaria and erythemas: urticaria, erythema multiforme; nodular lesions: erythema nodosum, vasiculitis reaction; telangiectatic and LE reactions; pigmentary reaction; other cutaneous reactions: fixed drug reactions, alopecia, hypertrichosis, macules, papules, angioedema, morbilliform-maculopapular rash, toxic epidermal necrolysis, erythema multiforme, erythema nodosum, contact dermititis, vesicles, petechiae, exfolliative dermititis, fixed drug eruptions, and severe skin rash (Stevens-Johnson syndrome).

Drugs known to be associated with cutaneous toxicities include, but are not exclusive of, antineoplastic agents, sulfonamides, hydantoins and others listed for each type of toxicity.

Uticaria and angioedema—is defined as the transient appearance of elevated, erythematous pruitic wheals (hives) or serpiginous exanthem. The appearance of uticaria is perceived as ongoing immediate hypersensitivity reaction. Angioedema is defined as uticaria, but involving deeper dermal and subdermal sites. Uticaria and angioedema appear to result from dilation of local postcapillary venules. Degranulation of cutaneous mast cells may be involved.

Drugs associated with uticaria and angioedema include, but are not excluded to, antimicrobials include, but not exclusive of, 5-aminosalicylic acid, aminoglycosides, cephalosporins, ethambutol, isoniazid, metronidazole, miconazole, nalidixic acid, penicillins, quinine, rifampin, spectinomycin, sulfonamides, and other drugs: asparaginase, aspirin and other non-steroidal antiinflammatory agenets, calcitonin, chloral hydrate, chlorambucil, cimetidine, cyclophosphamide, daunorubicin, ergotamine, ethchlorvynol, doxorubicin, ethosuximide, ethylenediamine, glucocorticoids, melphalan, penicillamine, phenothiazines, procainamide, procarbazine, quinidine, tartazine, thiazide diuretics, thiotepa.

Morbilliform-maculopapular rash—are rashes that result in eruptions or are morbilliform in nature.

Drugs associated with rashes include, but are not limited to, 5-aminosalicyclic acid, cephalosporins, erythromycin, gentamicin, penicillins, streptomycin, sulfonamides, allopurinol, barbiturates, captopril, coumarin, gold salts, hydantoins, thiazide diuretics.

Toxic epidermal necrolysis and erythroderma and exfoliative dermititis

Cutaneous erythroderma, edema, scaling, and fissuring may occur in response to certain drugs. Drugs associated with these types of cutaneous reactions include, but are limited to, allopurinol, amikacin, captopril, carbamazepine, chloral hydrate, chlorambucil, chloroquine, chlorpromazine, cyclosporine, diltiazem, ethambutol, ethylenediamine, glutethimide, gold salts, griseofulvin, hydantoins, hydroxychloroquine, minoxidil, nifedipine, nonsteroid antiinflammatory agents, penicillin, phenobarbital, rifampin, spironolactone, sulfonamides, trimethadione, trimethoprim, tocainamide, tocainide, vancomycin, verpamil.

Erythema mutliforme—is characterized by a hypersensitivity reaction in blood vessels of the dermis. The hypersensitivity is the result of immune complexes formed by small molecules interacting with proteinaceous components of the blood vessels. In cases whereby the mucosal membranes of the mouth and eye are involved, is referred to as Stevens-Johnson syndrome. Typically the cutaneous lesions, blisters and painful erosions occur in the mout and eye.

Drugs associated with erythema mulitforme include, but are not limited to, allopurinol, acetominophen, amikacin, barbiturates, carbamazepine, chloroquine, chlorporamide, clindamycin, ethambutol, ethosuximide, gold salts, glucocorticoids, hydantoins, hydralazine, hydroxyurea, mechlorethamine, meclofenamate, penicillins, phenothiazides, phenophthalein, phenylbutazone, rifampin, streptomycin, sulfonamides, sulfonylureas, sulindac, vaccines.

Fixed drug eruptions

Drug associated with fixed drug eruptions include, but are not excluded to, acetominophen, 5-aminosalicyclic acid, aspirin, barbiturates, benzodiazepines, barbiturates, chloroquine, dapsone, dimethylhydrinate, gold salts, hydralazine, hyoscine, ibuprofen, iodides, meprobamate, methanamine, metronidazole, penicillins, phenobarbital, phenolphthalein, phenothiazides, phenylbutazone, procarbazine, pseudoephedrine, quinine, saccharin, streptomycin, sulfonamides, and tetracyclines.

Erythema nodosum—is an innflammatory reaction in subcutaneous fat which represents a hypersentivity reaction to a number of antigenic stimuli. Multiple red, painful nodules do not ulcerate but involute and leave a yellow-purple bruises. Small molecules intreracting with proteinaceous components forma asensitizing antigen.

Drugs associated with producing erythema nododum include, but are not excluded to, bromides, oral contraceptives, penicillins, and sulfonamides.

Contact dermititis—is characterized by eruptions on histological analysis to epidermal intercellular edema (spongiosis). Contact dermititis can be caused by allergic or irritant mechanisms. Allergic contact dermititis is a delayed hypersensitivity reaction that can occur in response to a variety of small molecules that when bound to proteinaceous components of the skin form a sensitizing antigen. The antigen is processed by Langerhans' cells in the epidermis, presenting the antigen to the circulating T lymphocytes. Irritant dermititis is produced by substances that irritate or have a direct toxic effect on the skin.

Drugs associated with contact dermititis side effects include, but are not limited to, ambroxol, amikacin, antihistamines, bacitracin, benzalkonium chloride, benzocaine, benzyl chloride, cetl alcohol, chloramphenicol, chlorpormazine, clioquinol, colophony, ethylenediamine, fluorouracil, formaldehyde, gentamycin, glucocorticoids, glutaraldehyde, heparin, hexachlorophene, iodochlorhydroxyquin, lanolin, local anesthestics, minoxidil, naftin, neimycin, nitrofurazone, opiates, para-aminobenzoic acid, parabens, penicillins, phenothiazines, prolflavine, propylene glycol, streptomycin, sulfonamides, thimerosal, timolol.

Impact of Stratification Based Upon Genotype in Drug Development for Drugs, Compounds, or Candidate Therapeutic Interventions that May Induce Cutaneous Reactions

Recently, it has been described that there is a deletion polymorphism in the B2 bradykinin receptor gene (B2BKR). It was revealed that there is a 9 base pair deletion in exon 1 of the B2BKR gene and upon inspection of patients experienceing angioedema, patients with immunochemical evidence of angioedema were homozygous for no deletion at that site. These results were suggestive of B2BKR genotype influence on the clinical status and manifestation angioedema.

There is evidence to suggest that there are safety response differences to drug therapy in reference to development of cutaneous reactions which may be attributable to genotypic differences between individuals. There is provided in this invention examples of gene pathways that are implicated in the disease process or its therapy and those that potentially cause this variability. The Detailed Description above demonstrates how identification of a candidate gene or genes and gene pathways, stratification, clinical trial design, and implementation of genotyping for appropriate medical management of a given disease can be used to identify the genetic cause of variations in clinical response to therapy, new diagnostic tests, new therapeutic approaches for treating this disorder, and new pharmacuetical products or formulations for therapy. Gene pathways including, but not limited to, those that are outlined in the gene pathway Table 1, and pathway matrix Table 2 and discussed below are candidates for the genetic analysis and product development using the methods described above.

Advantages of Inclusion of Pharmacogenetic Stratification in Clinical Development of Agents that May Cause Cutaneous Reactions

The advantages of a clinical research and drug development program that includes the use of polymorphic genotyping for the stratification of patients for the appropriate selection of candidate therapeutic intervention includes 1) identification of patients that may respond earlier and show signs and symptoms of cutaneous reactions, 2) identification of the primary gene and relevant polymorphic variance that directly affects manifestation of a cutaneous disorder, 3) identification of pathophysiologic relevant variance or variances and potential therapies affecting those allelic genotypes or haplotypes, and 4) identification of allelic variances or haplotypes in genes that indirectly affects efficacy, safety or both.

By identifying subsets of patients, based upon genotype, that experience cutaneous reactions in response to the administration of a drug, agent or candidate therapeutic intervention, optimal selection may reduce level and extent of the skin damage. Appropriate genotyping and correlation to dosing regimen, or selection of optimal therapy would be beneficial to the patient, caregivers, medical personnel, and the patient's loved ones.

As an example of identification of the primary gene and relevant polymorphic variance that directly affects efficacy, safety, or both one could select an gene pathway as described in the Detailed Description, and determine the effect of genetic polymorphism and therapy efficacy, safety, or both within that given pathway. For example, referring to Table 2, genes involved in drug transport, phase I and phase II metabolism, protection from reactive intermediate damage, and immune responsiveness, the optimization of therapy of by an agent known to have a cutaneous side effect by determining whether the patient has a predisposing genotype in which the selected agents are more effective and or are more safe. In considering an optimization protocol, one could potentially predetermine the genotypic profile of these genes involved in the manifestation of the adverse effect, or those genes preeminently responsible for drug response. By embarking on the previously described gene pathway approach, it is technical feasibility to determine the relevant genes within such a targeted drug development program.

Identification of pathophysiologic relevant variance or variances and potential therapies affecting those allelic genotypes or haplotypes may speed drug development for therapeutic alternatives. There is a need for therapies that are targeted to a disease and symptom management with limited or no undesirable side effects. Identification of a specific variance or variances within genes involved in the pathophysiologic manifestation of cutaneous reactions and specific genetic polymorphisms of these critical genes may assist the development of novel agents and the identification of those patients that may best benefit from therapy of these candidate therapeutic alternatives.

By identifying allelic variances or haplotypes in genes that indirectly affects efficacy, safety of any class of drugs that has an effect on the prevention, progression, or symptoms of cutaneous reactions, one could target specific secondary drug or agent therapeutic actions that affect the overall therapeutic action.

Pharmacogenomics studies for these drugs, or other agents, compounds, or candidate therapeutic interventions, could be performed by identifying genes that are involved in the the function of a drug including, but not limited to absorption, distribution, metabolism, or elimination , the interaction of the drug with its target as well as potential alternative targets, the response of the cell to the binding of a drug to a target, the metabolism (including synthesis, biodistribution or elimination) of natural compounds which may alter the activity of the drug by complementary, competitive or allosteric mechanisms that potentiate or limit the effect of the drug, and genes involved in the etiology of the disease that alter its response to a particular class of therapeutic agents. It will be recognized to those skilled in the art that this broadly includes proteins involved in pharmacokinetics as well as genes involved in pharmacodynamics. This also includes genes that encode proteins homologous to the proteins believed to carry out the above functions are also worth evaluation as they may carry out similar functions. Together, the foregoing proteins constitute the candidate genes for affecting response of a patient to the therapeutic intervention. Using the methods described above, variances in these genes can be identified, and research and clinical studies can be performed to establish an association between a drug response or toxicity and specific variances.

### EXAMPLE 5

### Drug-Induced CNS Toxicity

Drug-induced central nervous system toxicity includes CNS stimulation or CNS depression. Characteristics of CNS toxicity include, but are not limited to, tinnitus and dizziness, acute dystonic reactions, parkinsonian syndrome, coma, convulsions, depression and psychosis, sweating, mydriasis, hyperpyrexia, centrally mediated cardiovascular involvement (hypertension, tachycardia, extrasystoles, arrythmias, circulatory collapse) and respiratory depression or tachypnea. Drugs known to be associated with CNS toxicity include, but are not exclusive of, salicylates, antipsychotics, sedatives, cholinergics,

Impact of Stratification Based Upon Genotype in Drug Development for Drugs, Compounds, or Candidate Therapeutic Interventions that May Induce CNS Toxicity

There is evidence to suggest that there are safety response differences to drug therapy in reference to development of CNS toxicities which may be attributable to genotypic differences between individuals. There is provided in this invention examples of gene pathways that are implicated in the disease process or its therapy and those that potentially cause this variability. The Detailed Description above demonstrates how identification of a candidate gene or genes and gene pathways, stratification, clinical trial design, and implementation of genotyping for appropriate medical management of a given disease can be used to identify the genetic cause of variations in clinical response to therapy, new diagnostic tests, new therapeutic approaches for treating this undesirable adverse effect, and new pharmacuetical products or formulations for therapy. Gene pathways including, but not limited to, those that are outlined in the gene pathway Table 1, and pathway matrix Table 2 and discussed below are candidates for the genetic analysis and product development using the methods described above.

Advantages of Inclusion of Pharmacogenetic Stratification in Clinical Development of Agents that May Cause CNS Toxicities

The advantages of a clinical research and drug development program that includes the use of polymorphic genotyping for the stratification of patients for the appropriate selection of candidate therapeutic intervention includes 1) identification of patients that may respond earlier and show signs and symptoms of CNS toxicities, 2) identification of the primary gene and relevant polymorphic variance that directly affects manifestation of a CNS toxicity, 3) identification of pathophysiologic relevant variance or variances and potential therapies affecting those allelic genotypes or haplotypes, and 4) identification of allelic variances or haplotypes in genes that indirectly affects efficacy, safety or both.

By identifying subsets of patients, based upon genotype, that experience CNS toxicity in response to the administration of a drug, agent or candidate therapeutic intervention, optimal selection may reduce level and extent of the neurologic damage. Appropriate genotyping and correlation to dosing regimen, or selection of optimal therapy would be beneficial to the patient, caregivers, medical personnel, and the patient's loved ones.

As an example of identification of the primary gene and relevant polymorphic variance that directly affects efficacy, safety, or both one could select an gene pathway as described in the Detailed Description, and determine the effect of genetic polymorphism and therapy efficacy, safety, or both within that given pathway. For example, referring to Table 2, genes involved in drug transport, phase I and phase II metabolism, protection from reactive intermediate damage, the optimization of therapy of by an agent known to inpart CNS toxic or undesirable side effect or effects by determining whether the patient has a predisposing genotype in which the selected agents are more effective and or are more safe. In considering an optimization protocol, one could potentially predetermine the genotypic profile of these genes involved in the manifestation of the adverse effect, or those genes preeminently responsible for drug response. By embarking on the previously described gene pathway approach, it is technical feasibility to determine the relevant genes within such a targeted drug development program.

Identification of pathophysiologic relevant variance or variances and potential therapies affecting those allelic genotypes or haplotypes may speed drug development for therapeutic alternatives. There is a need for therapies that are targeted to a disease and symptom management with limited or no undesirable side effects. Identification of a specific variance or variances within genes involved in the pathophysiologic manifestation of CNS toxicities and specific genetic polymorphisms of these critical genes may assist the development of novel agents and the identification of those patients that may best benefit from therapy of these candidate therapeutic alternatives.

By identifying allelic variances or haplotypes in genes that indirectly affects efficacy, safety of any class of drugs that has an effect on the prevention, progression, or symptoms of CNS toxicities, one could target specific secondary drug or agent therapeutic actions that affect the overall therapeutic action of neuroprotective agents.

Pharmacogenomics studies for these drugs, or other agent, compound, drug, or candidate therapeutic intervention, could be performed by identifying genes that are involved in the the function of a drug including, but not limited to absorption, distribution, metabolism, or elimination, the interaction of the drug with its target as well as potential alternative targets, the response of the cell to the binding of a drug to a target, the metabolism (including synthesis, biodistribution or elimination) of natural compounds which may alter the activity of the drug by complementary, competitive or allosteric mechanisms that potentiate or limit the effect of the drug, and genes involved in the etiology of the disease that alter its response to a particular class of therapeutic agents. It will be recognized to those skilled in the art that this broadly includes proteins involved in pharmacokinetics as well as genes involved in pharmacodynamics. This also includes genes that encode proteins homologous to the proteins believed to carry out the above functions are also worth evaluation as they may carry out similar functions. Together the foregoing proteins constitute the candidate genes for affecting response of a patient to the therapeutic intervention. Using the methods described above, variances in these genes can be identified, and research and clinical studies can be performed to establish an association between a drug response or toxicity and specific variances.

### EXAMPLE 6

### Drug-Induced Liver Toxicity

Drug-induced liver disease or drug-induced liver toxicity can manifest as zonal necrosis, nonspecific focal hepatitis, viral hepatitis-like reactions, inflammatory or noninflammatory cholestasis, small or large droplet fatty liver, granulomas, chronic hepatitis, fibrosis, tumors, or vascular lesions.

In the majority of the cases of known drug-induced liver toxicity, the drug is metabolized to a form that is deleterious to hepatic, or extrahepatic function. There are many endogenous or exogenous compounds that may be considered to attenuate or ablate toxic hepatocyte-produced metabolite mechanisms or effects of hepatic or extrahepatic damage.

In hepatocellular damage, free oxygen radicals may be generated in the hepatic metabolic processes that are deleterious to intracellular organelles, DNA, or metabolic pathways. There are endogenous cytoprotective agents that may prevent free radical-mediated damage such as retinoids, flavins, reduced glutathione, vitamin E,S-adenylylmethionine, and the enzyme superoxide dismutase (SOD). In animal models in which SOD activity is diminished or absent, the liver function was normal, but the sensitivity to toxin challenge was heightened.

In cholestatic damage, the bile salt uptake, metabolism, secretion, or transport is compromised and the residual increased bile salt concentrations are deleterious to hepatocyte function. The increase in bile salts is the main metabolic disturbance that initially leads to jaundice and pruritis and can progress to pancreatitis, hyperbilirubinemia, biliary cirrhosis, and hepatic encephalopathy.

In both cases of drug-induced liver toxicity, the drug must first be absorbed and enter in the hepatic circulation. Further, clinically it is often difficult to determine whether cholestatic damage leads to hepatocellular damage or whether hepatocellular damage leads to cholestatic damage. In many cases, until the patient is symptomatic, the underlying damage mechanisms may be clinically overlooked. By the time the drug-induced liver disease is symptomatic, the damage, be it hepatocellular or cholestatic or both, may be irreversible.

Identification of Genes involved in Drug-Induced Liver Toxicity

Thus, in the process of identifying drug- or xenobiotic-induced liver toxicity, one skilled in the art would identify key metabolic enzymes or bile cannicula transport processes that would be linked with either hepatocellular damage or cholestasis or combination of hepatocellular damage or cholestasis.

Hepatocellular damage may be the result of direct chemical mediated effects, may be severe, and usually is associated with damage within organelles, DNA and membranes. Clinically there is a marked elevation of SGOT and SGPT as well as other enzymes. In cases of cholestasis there is jaundice, pruritis, a marked elevation of bile salts and alkaline phosphatase activity, but not an elevation of SGOT or SGPT. In cases of toxic liver disease there is difficulty, at least initially to determine the underlying etiology. Clinically, symptoms may not appear as clear as described above. Further, depending on the rate and extent of the damage, hepatocellular damage may be masked or asymptomatic until liver impairment has induced cholestasis.

Potentially hepatotoxic agents can be divided broadly into two groups: intrinsic hepatotoxins and idiosyncratic hepatotoxins. Intrinsic hepatotoxins produce acute liver damage in a predictable, dose-dependent fashion shortly after ingestion or exposure. Generally, all subjects exposed will uniformly exhibit signs and symptoms. In this category, the effects seen in humans can be mimicked in animal models. Examples of intrinsic hepatotoxins are carbon tetrachloride, 2-nitropropane, trichloroethane, the octapeptide toxins of the Amanita mushroom species, and the antipyretic, acetominophen. In some of these cases, toxic metabolites result in covalent modification of hepatocyte macromolecules or reactive oxygen intermediates leads to peroxidation of cell membrane lipids or other intracellular molecules.

In contrast, idiosyncratic hepatotoxins produce liver damage in an unpredictable, dose-independent manner after a latent period of ingestion or exposure. Animal models or experimental data is generally incapable of predicting the effect in humans. Further, idiosyncratic hepatotoxins do not uniformly affect a population; a subset of the group exposed may or may not exhibit signs or symptoms. Range of symptoms are from mild to severe and is thought to coincide with differences in the pathways of drug or xenobiotic biotransformation or immune-mediated drug sensitivity (drug allergy). In idiosyncratic drug-induced liver disease, fever, arthralgias, rash, eosinophilia, are often prominent and indicate a hypersensitivity reaction.

Impact of Stratification Based Upon Genotype in Drug Development for Drugs, Compounds, or Candidate Therapeutic Interventions that may Induce Hepatotoxicity

Genes encoding proteins with catalytic function that are involved in the metabolism of drugs or xenobiotics are listed in Tables 1 and 2 below. Further listed are those proteins that are involved in the uptake, transport, or secretion into the bile cannicula. Below are further specific example of drug-specific effects on the liver.

Acetaminophen-Induced Liver Disease

Acetominophen is a readily available, easy to administer analgesic that is an example of a intrinsic hepatotoxin. This hepatotoxin causes zonal necrosis and acute liver failure and is associated with renal failure. Although a high dose (10-15 grams) is required for significant liver injury to occur, the onset of initial symptoms does not occur until hours after ingestion. The progression of symptoms occurs including progressive liver failure with hepatic encephalopathy, prolongation of prothrombin time, hypoglycemia, and lactic acidosis. The liver injury is caused by a toxic metabolite of acetominophen via the P450 metabolizing system. This toxic intermediate at low concentrations is conjugated with glutathione. However, in toxic doses, the conjugating enzymes stores are exhausted and the reactive intermediate reacts with intracellular proteins and results in cellular dysfunction and ultimately death. The rate of metabolism is dependent on the concentrations of both P450 and glutathione. Speeding this toxic pathway may include increasing the available P450 or reducing the availablility of glutathione, e.g. using known inducers of P450 such as ethanol and and phenobarbital; and known inhibitors of glutathione concentrations, e.g., ethanol and fasting. Acetominophen toxicity is completely reversed if the drug is removed. Chronic ingestion may produce subclinical liver injury, centrilobular necrosis, or chronic hepatitis; however all reversible if the drug is removed.

Amiodarone-Induced Liver Disease

Amiodarone is used in treatment of refractory arrythmias. In some patients amiodarone produces mild to moderate increases of serum transaminases which are generally accompanied by engorgement of lysosomes with phospholipid. In a fraction of the patients, a more severe liver injury develops which histologically resembles alcoholic hepatitis: fat infiltration of hepatocytes, focal necrosis, fibrosis, polymorphonuclear leukocyte infiltrates, and Mallory bodies. The lesion may progress to micronodular cirrhosis, with portal hypertension and liver failure. Hepatomegaly is seen, but jaundice is rare.

Amiodarone accumulates in lysosomes and inhibits lysosomal phopholipases, however the connection between this mechanism and alcoholic hepatitis histopathology is unknown. Unfortunately, rapid discontinuation of amiodarone increases the risk of cardiac arrythmias.

Chlopromazine-Induced Liver Disease

Chlorpromazine is an anti-psychotic agent which, in a small portion of the patient population can produce a cholestatic reaction. Symptoms include fever, anorexia, arthalgias, pruritis, jaundice, and eosinophilia is common. This idiosyncratic type of liver toxicity suggests a hypersensitivity type reaction. The symptoms subside over a period of weeks following discontinuation, Rarely, residual cholestatic disease occurs, treatment for pruritis and fat-soluble vitamin supplementation may be required, but eventual recovery almost always occurs.

Erythromycin-Induced Liver Disease

Erythromycin, a broad spectrum antibiotic, can be accompanied by a cholestatic reaction. Inflammatory cell infiltration and liver cell necrosis may occur. The hepatoptoxicity presents as right upper quadrant pain, fever, and variable cholestatic symptoms. The prognosis is uniform and will occur after readminstration of the drug, The mechanism of action is unknown.

Halothane-Induced Liver Disease

Halothane is a gaseous anthesthetic and can, in rare instances, cause a viral-like hepatitis syndrome. In severe cases, this hepatotoxicity, may cause fatal massive heaptic necrosis. Severe reactions seem to appear after previous or multiple exposure to halothane. It is known that the P450 metabolites of this xenobiotic are responsible for the mechanism of hepatic injury.

Isoniazid (INH)-Induced Liver Disease

Isoniazid is used as a single drug in the prophylaxis of tuberculosis. In 10-20% of of the persons taking INH, subclinical liver injury occurs. The conversion of INH to acetylhydrazine is via acetylation. In slow acetylators, INH is more hepatotoxic. The conversion of INH to acetylhydrazine to diacetylhydrazine is impaired. In slow acetylators, the acetylhydrazine is not well metabolized and is further oxidized by one of the P450 enzymes to a toxic, reactive molecule that is responsible for the liver disease. Discontinuation of the drug returns the enzymatic levels to normal and the liver is able to restore activity.

Sodium Valproate-Induced Liver Disease

Sodium valproate is an anti-epileptic agent that is routinely prescribed for petit mal epilepsy and in some cases produces severe hepatotoxicity. Similar to INH, sodium valproate is accompanied by a high incidence of transient, slight and asymptomatic increases in serum transaminases. Usually the increased enzyme activity appears after weeks of treatment. In rare cases of severe liver toxicity, the nonspecific systemic and digestive symptoms are followed by jaundice, evidence of liver failure, as well as encephalopathy and coagulopathy. The mechanism of hepatotoxicity is unknown, however there are theories that there is impairment of mitochondiral oxidation of long-chain fatty acids by a metabolite of the parent drug. Symptoms subside with little to no residual liver dysfunction after discontinuing the drug.

Oral Contraceptive Induced Liver Disease

Estrogen, progesterone, and combination oral contraceptives can produce several adverse effects on the heptobiliary system. They are 1) hepatocellular cholestasis, 2) liver cell neoplasias, 3) increased predisposition to cholesterol and gall stone formation, 4) hepatic vein thrombosis. These cholestatic hepatotoxic effects are attributed to estrogen's direct effect on bile formation. The mechanism of action is unknown.

There is evidence to suggest that there are safety response differences to drug therapy in reference to development of drug-induced liver toxicity which may be attributable to genotypic differences between individuals. There is provided in this invention examples of gene pathways that are implicated in the disease process or its therapy and those that potentially cause this variability. The Detailed Description above demonstrates how identification of a candidate gene or genes and gene pathways, stratification, clinical trial design, and implementation of genotyping for appropriate medical management of a given disease can be used to identify the genetic cause of variations in clinical response to therapy, new diagnostic tests, new therapeutic approaches for treating this disorder, and new pharmacuetical products or formulations for therapy. Gene pathways including, but not limited to, those that are outlined in the gene pathway Table 1, and pathway matrix Table 2 and discussed below are candidates for the genetic analysis and product development using the methods described above.

Advantages of Inclusion of Pharmacogenetic Stratification in Clinical Development of Agents that May Cause Liver Toxicity

The advantages of a clinical research and drug development program that includes the use of polymorphic genotyping for the stratification of patients for the appropriate selection of candidate therapeutic intervention includes 1) identification of patients that may respond earlier and show signs and symptoms of liver toxicity, 2) identification of the primary gene and relevant polymorphic variance that directly affects manifestation of a liver disorder, 3) identification of pathophysiologic relevant variance or variances and potential therapies affecting those allelic genotypes or haplotypes, and 4) identification of allelic variances or haplotypes in genes that indirectly affects efficacy, safety or both.

By identifying subsets of patients, based upon genotype, that experience drug-induced liver toxicity in response to the administration of a drug, agent or candidate therapeutic intervention, optimal selection may reduce level and extent of the hepatic damage. Appropriate genotyping and correlation to dosing regimen, or selection of optimal therapy would be beneficial to the patient, caregivers, medical personnel, and the patient's loved ones.

As an example of identification of the primary gene and relevant polymorphic variance that directly affects efficacy, safety, or both one could select an gene pathway as described in the Detailed Description, and determine the effect of genetic polymorphism and therapy efficacy, safety, or both within that given pathway. For example, referring to Table 2, genes involved in drug transport, phase I and phase II metabolism, excretion, hepatic cannicular uptake and concentration, and protection from reactive intermediate damage the optimization of therapy by an agent known to have a hepatic side effect by determining whether the patient has a predisposing genotype in which the selected agents are more effective and or are more safe. In considering an optimization protocol, one could potentially predetermine the genotypic profile of these genes involved in the manifestation of the adverse effect, or those genes preeminently responsible for drug response. By embarking on the previously described gene pathway approach, it is technical feasibility to determine the relevant genes within such a targeted drug development program.

Identification of pathophysiologic relevant variance or variances and potential therapies affecting those allelic genotypes or haplotypes may speed drug development for therapeutic alternatives. There is a need for therapies that are targeted to a disease and symptom management with limited or no undesirable side effects. Identification of a specific variance or variances within genes involved in the pathophysiologic manifestation of drug-induced liver toxicity and specific genetic polymorphisms of these critical genes may assist the development of novel agents and the identification of those patients that may best benefit from therapy of these candidate therapeutic alternatives.

By identifying allelic variances or haplotypes in genes that indirectly affects efficacy, safety of any class of drugs that has an effect on the prevention, progression, or symptoms of drug induced liver toxicity, one could target specific secondary drug or agent therapeutic actions that affect the overall therapeutic action of hepatoprotective agents.

Pharmacogenomics studies for these drugs, or other agent, compound, drug, or candidate therapeutic intervention, could be performed by identifying genes that are involved in the the function of a drug including, but not limited to absorption, distribution, metabolism, or elimination, the interaction of the drug with its target as well as potential alternative targets, the response of the cell to the binding of a drug to a target, the metabolism (including synthesis, biodistribution or elimination) of natural compounds which may alter the activity of the drug by complementary, competitive or allosteric mechanisms that potentiate or limit the effect of the drug, and genes involved in the etiology of the disease that alter its response to a particular class of therapeutic agents. It will be recognized to those skilled in the art that this broadly includes proteins involved in pharmacokinetics as well as genes involved in pharmacodynamics. This also includes genes that encode proteins homologous to the proteins believed to carry out the above functions are also worth evaluation as they may carry out similar functions. Together the foregoing proteins constitute the candidate genes for affecting response of a patient to the therapeutic intervention. Using the methods described above, variances in these genes can be identified, and research and clinical studies can be performed to establish an association between a drug response or toxicity and specific variances.

### EXAMPLE 7

### Drug-Induced Cardiovascular Toxicity

Drug induced cardiovascular toxicities include but are not excluded to arrythmias, tachycardia, extrasystoles, circulatory collapse, QT prolongation, cardiomyopathy, hypotension, or hypertension. Drugs known to elicit these type of responses include but are not excluded to theophylline, hydantoins, doxorubicin, daunorubicin.

Arrythmias—If the normal sequence of electrical impulse and propagation through myocardial tissue is perturbed, an arrythmia occurs. Broadly, arrythmias fall into one of three categories: bradyarrythmias (slowing or failure of the initiating impulse), heart block (an impaired propagation through node tissue or atrial or ventricular muscle), and tachyarrythmias (abnormal rapid heart rhythms). Subcategories include: sinus bradycardia, atrioventricular block (AV block), sinus tachycardia, ventricular tachycardia, atrial flutter, multifocal atrial tachycardia, polymorphic ventricular tachycardia with or without QT prolongation, frequent or difficult to terminate ventricular tachycardia, atrial tachycardia with or without AV block, ventricular bigeminy, and ventricular fibrillation. Drugs known to induce these types of arrythmias include, but are not excluded to, digitalis, verapamil, diltiazem, b-adrenergic blockers, clonidine, methyldopa, quinidine, flecainide, propafenone, theophylline, sotalol, procainamide, disopyramide, certain non-cardioactive drugs ( ), and amiodarone.

Heart Rate, Tachycardia-Heart rate is under both sympathetic and parasympathic control. The influence of heart rate on cardiac output is paramount. Drugs affecting heart rate include, but are not limited to, sympathomimetics, parasympathomimetics, and agents or compounds affecting these two central inputs.

Extasystoles—is defined as premature myocardial excitation. Extrasystoles can include atrial, nodal, or ventricular. Other asynchronous pathologies may result from these systoles. Drugs known to be associated with extra systoles include, but are not excluded to, agents that prolong the depolarization time, agents that leave a residual available intracellular calcium, or agents that alter the function of the K+ or Na+ channel activity.

QT Prolongation—is the interval on an electrocardiogram that indicates ventricular action potential duration. QT prolongation can lead to uncoordinated atrial and ventricular action potentials. In these circumstances of delayed or prolonged polymorphic ventricular after depolarizations, resultant abnormal triggering of secondary, uncoordinated depolarizations can occur. Two of these conditions are explained as follows and may be associated with underlying rapid or slow heart rate: 1) under conditions of residual excess intracellular calcium (myocardial ischemia, adrenergic stress, digitalis intoxication), and 2) under conditions of marked prolongation of cardiac action potential (agents (antiarrythmics or others) that prolong action potential duration).

Cardiomyopathy—There are broadly three categories of cardiomyopathies: dilated, hypertrophic, and restrictive. These cardiac muscular diseases can be of mechanical or acquired origin.

Dilated cardiomyopathies are generally caused by myocardial injury that results in depressed systolic function and progressive ventricular dilatation. Drug induced dilated cardiomyopathy can occur in the presence of, but are not excluded to, ethanol, chenotherapeutic agents, elemental compounds, and catecholamimetics.

Hypertrophic cardiomyopathy is the presentation of grossly assymetric (eccentric) or symmetric (concentric) hyoertrophy of the left ventricle in the absence of another cardiac or systemic disease capable of producing the disproportionate increase in ventricle mass. In drug induced hypertrophic cardiomyopathy, there may be compensatory hypertrophy of the left ventricle in response to inordinate and or sustained hypertension or prolonged reduced or insufficient cardiac output as a result of myocardial injury or noncardiac mediated physiological events.

Restrictive cardiomyopathies are the result of a primary abnormality of diastolic function (impaired filling). Impaired diastolic function can occur as a result of morphologically detectable myocardial or endomyocardial disease, interstitial deposition of deposition of abnormal substances (infiltrative), intracellular accumulation of abnormal substances (strage diseases), or as a result of endomyocardial disease. In the last category, anthracyclines have been associated with both dilated and restrictive cardiomyopathies.

Blood Pressure—Blood pressure is regulated in a complex interplay of neural and endocrine mechanisms. These mechanisms are aimed at the physiologic contorl of cardiac output, delivery of blood components to the tissues, and removal of metabolic by-products from the tissues.

Hypertension is defined as the elevated arterial blood pressure either an increase of systolic or diastolic pressure or both. Secondary hypertension can be associated with drugs and chemicals including, but not limited to, cyclosporine, oral contraceptives, glucocorticoids, mineralocorticoids, sympathomimetics, tyramine, and MAO inhibitors.

Hypotension is defined as the reduction in blood pressure that is associated with orthostatic hypotension, syncope, head injury, hepatic failure, antidiuresis, myocardial infarction and cardiogenic shock. Drug-induced hypotension is associated drugs including, but not exclusive of, parasympathomimetics, diuretics, and direct acting cardiac agents.

Impact of Stratification Based Upon Genotype in Drug Development for Drugs, Compounds, or Candidate Therapeutic Interventions that may Induce Cardiovascular Toxicity

There is evidence to suggest that there are safety response differences to drug therapy in reference to development of cardiovascular toxicity which may be attributable to genotypic differences between individuals. There is provided in this invention examples of gene pathways that are implicated in the disease process or its therapy and those that potentially cause this variability. The Detailed Description above demonstrates how identification of a candidate gene or genes and gene pathways, stratification, clinical trial design, and implementation of genotyping for appropriate medical management of a given disease can be used to identify the genetic cause of variations in clinical response to therapy, new diagnostic tests, new therapeutic approaches for treating this disorder, and new pharmacuetical products or formulations for therapy. Gene pathways including, but not limited to, those that are outlined in the gene pathway Table 1, and pathway matrix Table 2 and discussed below are candidates for the genetic analysis and product development using the methods described above.

Advantages of Inclusion of Pharmacogenetic Stratification in Clinical Development of Agents that May Cause Cardiovascular Toxicity

The advantages of a clinical research and drug development program that includes the use of polymorphic genotyping for the stratification of patients for the appropriate selection of candidate therapeutic intervention includes 1) identification of patients that may respond earlier and show signs and symptoms of cardiovascular toxicity, 2) identification of the primary gene and relevant polymorphic variance that directly affects manifestation of a cardiovascular disorder, 3) identification of pathophysiologic relevant variance or variances and potential therapies affecting those allelic genotypes or haplotypes, and 4) identification of allelic variances or haplotypes in genes that indirectly affects efficacy, safety or both.

By identifying subsets of patients, based upon genotype, that experience cardiovascular toxicities in response to the administration of a drug, agent or candidate therapeutic intervention, optimal selection may reduce level and extent of the cardiovascular damage. Appropriate genotyping and correlation to dosing regimen, or selection of optimal therapy would be beneficial to the patient, caregivers, medical personnel, and the patient's loved ones.

As an example of identification of the primary gene and relevant polymorphic variance that directly affects efficacy, safety, or both one could select an gene pathway as described in the Detailed Description, and determine the effect of genetic polymorphism and therapy efficacy, safety, or both within that given pathway. For example, referring to Table 2, genes involved in drug transport, phase I and phase II metabolism, and protection from reactive intermediate damage the optimization of therapy of by an agent known to have a cardiovascular side effect by determining whether the patient has a predisposing genotype in which the selected agents are more effective and or are more safe. In considering an optimization protocol, one could potentially predetermine the genotypic profile of these genes involved in the manifestation of the adverse effect, or those genes preeminently responsible for drug response. By embarking on the previously described gene pathway approach, it is technical feasibility to determine the relevant genes within such a targeted drug development program.

Identification of pathophysiologic relevant variance or variances and potential therapies affecting those allelic genotypes or haplotypes may speed drug development for therapeutic alternatives. There is a need for therapies that are targeted to a disease and symptom management with limited or no undesirable side effects. Identification of a specific variance or variances within genes involved in the pathophysiologic manifestation of cardiovascular toxicities and specific genetic polymorphisms of these critical genes may assist the development of novel agents and the identification of those patients that may best benefit from therapy of these candidate therapeutic alternatives.

By identifying allelic variances or haplotypes in genes that indirectly affects efficacy, safety of any class of drugs that has an effect on the prevention, progression, or symptoms of cardiovascular toxicities, one could target specific secondary drug or agent therapeutic actions that affect the overall therapeutic action of cardiovascular protective agents.

Pharmacogenomics studies for these drugs, or other agent, compound, drug, or candidate therapeutic intervention, could be performed by identifying genes that are involved in the the function of a drug including, but not limited to absorption, distribution, metabolism, or elimination, the interaction of the drug with its target as well as potential alternative targets, the response of the cell to the binding of a drug to a target, the metabolism (including synthesis, biodistribution or elimination) of natural compounds which may alter the activity of the drug by complementary, competitive or allosteric mechanisms that potentiate or limit the effect of the drug, and genes involved in the etiology of the disease that alter its response to a particular class of therapeutic agents. It will be recognized to those skilled in the art that this broadly includes proteins involved in pharmacokinetics as well as genes involved in pharmacodynamics. This also includes genes that encode proteins homologous to the proteins believed to carry out the above functions are also worth evaluation as they may carry out similar functions. Together the foregoing proteins constitute the candidate genes for affecting response of a patient to the therapeutic intervention. Using the methods described above, variances in these genes can be identified, and research and clinical studies can be performed to establish an association between a drug response or toxicity and specific variances.

### EXAMPLE 8

### Drug-Induced Pulmonary Toxicity

Drug induced pulmonary toxicity includes, but is not excluded to, asthma, acute pneumonitis, eosinophilic pneumonitis, fibrotic and pleural reactions, and interstitial fibrosis. Drug know to elicit pulmonary toxicity include, but are not excluded to, salicylates, nitrofuratoin, busulfan, nitrofurantoin, and bleomycin.

Impact of Stratification Based Upon Genotype in Drug Development for Drugs, Compounds, or Candidate Therapeutic Interventions that may Induce Pulmonary Toxicities

There is evidence to suggest that there are safety response differences to drug therapy in reference to development of pulmonary toxicities which may be attributable to genotypic differences between individuals. There is provided in this invention examples of gene pathways that are implicated in the disease process or its therapy and those that potentially cause this variability. The Detailed Description above demonstrates how identification of a candidate gene or genes and gene pathways, stratification, clinical trial design, and implementation of genotyping for appropriate medical management of a given disease can be used to identify the genetic cause of variations in clinical response to therapy, new diagnostic tests, new therapeutic approaches for treating this disorder, and new pharmacuetical products or formulations for therapy. Gene pathways including, but not limited to, those that are outlined in the gene pathway Table 1, and pathway matrix Table 2 and discussed below are candidates for the genetic analysis and product development using the methods described above.

Advantages of Inclusion of Pharmacogenetic Stratification in Clinical Development of Agents that May Cause Pulmonary Toxicities

The advantages of a clinical research and drug development program that includes the use of polymorphic genotyping for the stratification of patients for the appropriate selection of candidate therapeutic intervention includes 1) identification of patients that may respond earlier and show signs and symptoms of pulmonary toxicity, 2) identification of the primary gene and relevant polymorphic variance that directly affects manifestation of a pulmonary disorder, 3) identification of pathophysiologic relevant variance or variances and potential therapies affecting those allelic genotypes or haplotypes, and 4) identification of allelic variances or haplotypes in genes that indirectly affects efficacy, safety or both.

By identifying subsets of patients, based upon genotype, that experience pulmonary toxicities in response to the administration of a drug, agent or candidate therapeutic intervention, optimal selection may reduce level and extent of the pulmonary damage. Appropriate genotyping and correlation to dosing regimen, or selection of optimal therapy would be beneficial to the patient, caregivers, medical personnel, and the patient's loved ones.

As an example of identification of the primary gene and relevant polymorphic variance that directly affects efficacy, safety, or both one could select an gene pathway as described in the Detailed Description, and determine the effect of genetic polymorphism and therapy efficacy, safety, or both within that given pathway. For example, referring to Table 2, genes involved in drug transport, phase I and phase II metabolism, excretion, protection from reactive intermediate damage, and immune responsiveness, the optimization of therapy of by an agent known to have a pulmonary side effect by determining whether the patient has a predisposing genotype in which the selected agents are more effective and or are more safe. In considering an optimization protocol, one could potentially predetermine the genotypic profile of these genes involved in the manifestation of the adverse effect, or those genes preeminently responsible for drug response. By embarking on the previously described gene pathway approach, it is technical feasibility to determine the relevant genes within such a targeted drug development program.

Identification of pathophysiologic relevant variance or variances and potential therapies affecting those allelic genotypes or haplotypes may speed drug development for therapeutic alternatives. There is a need for therapies that are targeted to a disease and symptom management with limited or no undesirable side effects. Identification of a specific variance or variances within genes involved in the pathophysiologic manifestation of pulmonary toxicity and specific genetic polymorphisms of these critical genes may assist the development of novel agents and the identification of those patients that may best benefit from therapy of these candidate therapeutic alternatives.

By identifying allelic variances or haplotypes in genes that indirectly affects efficacy, safety of any class of drugs that has an effect on the prevention, progression, or symptoms of pulmonary toxicity, one could target specific secondary drug or agent therapeutic actions that affect the overall therapeutic action of pulmonary protective agents.

Pharmacogenomics studies for these drugs, or other agent, compound, drug, or candidate therapeutic intervention, could be performed by identifying genes that are involved in the the function of a drug including, but not limited to absorption, distribution, metabolism, or elimination, the interaction of the drug with its target as well as potential alternative targets, the response of the cell to the binding of a drug to a target, the metabolism (including synthesis, biodistribution or elimination) of natural compounds which may alter the activity of the drug by complementary, competitive or allosteric mechanisms that potentiate or limit the effect of the drug, and genes involved in the etiology of the disease that alter its response to a particular class of therapeutic agents. It will be recognized to those skilled in the art that this broadly includes proteins involved in pharmacokinetics as well as genes involved in pharmacodynamics. This also includes genes that encode proteins homologous to the proteins believed to carry out the above functions are also worth evaluation as they may carry out similar functions. Together the foregoing proteins constitute the candidate genes for affecting response of a patient to the therapeutic intervention. Using the methods described above, variances in these genes can be identified, and research and clinical studies can be performed to establish an association between a drug response or toxicity and specific variances.

### EXAMPLE 9

### Drug-Induced Renal Toxicity

Drug-induced renal toxicity includes, but is not exclusived to, glomerulonephritis and tubular necrosis. Drugs associated with eliciting renal toxicity include, but are not excluded to, penicillamine, aminoglycoside antibiotics, cyclosporine, amphotericin B, phenacetin, and salicylates.

Impact of Stratification Based Upon Genotype in Drug Development for Drugs, Compounds, or Candidate Therapeutic Interventions that may InduceRenal Toxicity

There is evidence to suggest that there are safety response differences to drug therapy in reference to development of renal toxicity which may be attributable to genotypic differences between individuals. There is provided in this invention examples of gene pathways that are implicated in the disease process or its therapy and those that potentially cause this variability. The Detailed Description above demonstrates how identification of a candidate gene or genes and gene pathways, stratification, clinical trial design, and implementation of genotyping for appropriate medical management of a given disease can be used to identify the genetic cause of variations in clinical response to therapy, new diagnostic tests, new therapeutic approaches for treating this disorder, and new pharmacuetical products or formulations for therapy. Gene pathways including, but not limited to, those that are outlined in the gene pathway Table 1, and pathway matrix Table 2 and discussed below are candidates for the genetic analysis and product development using the methods described above.

Advantages of Inclusion of Pharmacogenetic Stratification in Clinical Development of Agents that May Cause or are Associated with Renal Toxicity

The advantages of a clinical research and drug development program that includes the use of polymorphic genotyping for the stratification of patients for the appropriate selection of candidate therapeutic intervention includes 1) identification of patients that may respond earlier and show signs and symptoms of renal toxicity, 2) identification of the primary gene and relevant polymorphic variance that directly affects manifestation of a renal disorder, 3) identification of pathophysiologic relevant variance or variances and potential therapies affecting those allelic genotypes or haplotypes, and 4) identification of allelic variances or haplotypes in genes that indirectly affects efficacy, safety or both.

By identifying subsets of patients, based upon genotype, that experience renal toxicities in response to the administration of a drug, agent or candidate therapeutic intervention, optimal selection may reduce level and extent of the renal damage. Appropriate genotyping and correlation to dosing regimen, or selection of optimal therapy would be beneficial to the patient, caregivers, medical personnel, and the patient's loved ones.

As an example of identification of the primary gene and relevant polymorphic variance that directly affects efficacy, safety, or both one could select an gene pathway as described in the Detailed Description, and determine the effect of genetic polymorphism and therapy efficacy, safety, or both within that given pathway. For example, referring to Table 2, genes involved in drug transport, phase I and phase II metabolism, and renal tubular uptake and concentration the optimization of therapy of by an agent known to have a renal side effect by determining whether the patient has a predisposing genotype in which the selected agents are more effective and or are more safe. In considering an optimization protocol, one could potentially predetermine the genotypic profile of these genes involved in the manifestation of the adverse effect, or those genes preeminently responsible for drug response. By embarking on the previously described gene pathway approach, it is technical feasibility to determine the relevant genes within such a targeted drug development program.

Identification of pathophysiologic relevant variance or variances and potential therapies affecting those allelic genotypes or haplotypes may speed drug development for therapeutic alternatives. There is a need for therapies that are targeted to a disease and symptom management with limited or no undesirable side effects. Identification of a specific variance or variances within genes involved in the pathophysiologic manifestation of renal toxicity and specific genetic polymorphisms of these critical genes may assist the development of novel agents and the identification of those patients that may best benefit from therapy of these candidate therapeutic alternatives.

By identifying allelic variances or haplotypes in genes that indirectly affects efficacy, safety of any class of drugs that has an effect on the prevention, progression, or symptoms of renal toxicity, one could target specific secondary drug or agent therapeutic actions that affect the overall therapeutic action of renal protective agents.

Pharmacogenomics studies for these drugs, or other agent, compound, drug, or candidate therapeutic intervention, could be performed by identifying genes that are involved in the the function of a drug including, but not limited to absorption, distribution, metabolism, or elimination , the interaction of the drug with its target as well as potential alternative targets, the response of the cell to the binding of a drug to a target, the metabolism (including synthesis, biodistribution or elimination) of natural compounds which may alter the activity of the drug by complementary, competitive or allosteric mechanisms that potentiate or limit the effect of the drug, and genes involved in the etiology of the disease that alter its response to a particular class of therapeutic agents. It will be recognized to those skilled in the art that this broadly includes proteins involved in pharmacokinetics as well as genes involved in pharmacodynamics. This also includes genes that encode proteins homologous to the proteins believed to carry out the above functions are also worth evaluation as they may carry out similar functions. Together the foregoing proteins constitute the candidate genes for affecting response of a patient to the therapeutic intervention. Using the methods described above, variances in these genes can be identified, and research and clinical studies can be performed to establish an association between a drug response or toxicity and specific variances.

### EXAMPLE 10

### Hardy-Weinberg Equilibrium

Evolution is the process of change and diversification of organisms through time, and evolutionary change affects morphology, physiology and reproduction of organisms, including humans. These evolutionary changes are the result of changes in the underlying genetic or hereditary material. Evolutionary changes in a group of interbreeding individuals or Mendelian population, or simply populations, are described in terms of changes in the frequency of genotypes and their constituent alleles. Genotype frequencies for any given generation is the result of the mating among members (genotypes) of their previous generation. Thus, the expected proportion of genotypes from a random union of individuals in a given population is essential for describing the total genetic variation for a population of any species. For example, the expected number of genotypes that could form from the random union of two alleles, A and a, of a gene are AA, Aa and aa. The expected frequency of genotypes in a large, random mating population was discovered to remain constant from generation to generation; or achieve Hardy-Weinberg equilibrium, named after its discoverers. The expected genotypic frequencies of alleles A and a (AA, 2Aa, aa) are conventionally described in terms of p²+2pq+q² in which p and q are the allele frequencies of A and a. In this equation (p²+2pq+q²=1), p is defined as the frequency of one allele and q as the frequency of another allele for a trait controlled by a pair of alleles (A and a). In other words, p equals all of the alleles in individuals who are homozygous dominant (AA) and half of the alleles in individuals who are heterozygous (Aa) for this trait. In mathematical terms, this is

p=AA+½Aa

Likewise, q equals the other half of the alleles for the trait in the population, or

q=aa+½Aa

Because there are only two alleles in this case, the frequency of one plus the frequency of the other must equal 100%, which is to say

p+q=1

Alternatively,

p=1−q OR q=1−p

All possible combinations of two alleles can be expressed as:

(p+q)²=1

or more simply,

p²+2pq+q²=1

In this equation, if p is assumed to be dominant, then p² is the frequency of homozygous dominant (AA) individuals in a population, 2pq is the frequency of heterozygous (Aa) individuals, and q² is the frequency of homozygous recessive (aa) individuals.

From observations of phenotypes, it is usually only possible to know the frequency of homozygous dominant or recessive individuals, because both dominant and recessives will express the distinguishable traits. However, the Hardy-Weinberg equation allows us to determine the expected frequencies of all the genotypes, if only p or q is known. Knowing p and q, it is a simple matter to plug these values into the Hardy-Weinberg equation (p²+2pq+q²=1). This then provides the frequencies of all three genotypes for the selected trait within the population. This illustration shows Hardy-Weinberg frequency distributions for the genotypes AA, Aa, and aa at all values for frequencies of the alleles, p and q. It should be noted that the proportion of heterozygotes increases as the values of p and q approach 0.5.

Linkage Disequilibirum

Linkage is the tendency of genes or DNA sequences (e.g. SNPs) to be inherited together as a consequence of their physical proximity on a single chromosome. The closer together the markers are, the lower the probability that they will be separated during DNA crossing over, and hence the greater the probability that they will be inherited together. Suppose a mutational event introduces a “new” allele in the close proximity of a gene or an allele. The new allele will tend to be inherited together with the alleles present on the “ancestral,” chromosome or haplotype. However, the resulting association, called linkage disequilibrium, will decline over time due to recombination. Linkage disequilibrium has been used to map disease genes. In general, both allele and haplotype frequencies differ among populations. Linkage disequilibrium is varied among the populations, being absent in some and highly significant in others.

Quantification of the Relative Risk of Observable Outcomes of a Pharmacogenetics Trial

Let PlaR be the placebo response rate (0% ( PlaR ( 100%) and TntR be the treatment response rate (0% ( TntR ( 100%) of a classical clinical trial. ObsRR is defined as the relative risk between TntR and PlaR:

ObsRR=TntR/PlaR.

Suppose that in the treatment group there is a polymorphism in relation to drug metabolism such as the treatment response rate is different for each genotypic subgroup of patients. Let q be the allele a frequency of a recessive biallelic locus (e.g. SNP) and p=1−q the allele A frequency. Following Hardy-Weinberg equilibrium, the relative frequency of homozygous and heterozygous patients are as follow:

| | | |
|---|---|---|
| AA: p2 | Aa: 2pq | aa: q2 |

with

(p2+2pq+q2)=1.

Let's define AAR, AaR, aaR as respectively the response rates of the AA, Aa and aa patients. We have the following relationship:

TntR=AAR*p2+AaR*2pq+aaR*q2.

Suppose that the aa genotypic group of patients has the lowest response rate, i.e. a response rate equal to the placebo response rate (which means that the polymorphism has no impact on natural disease evolution but only on drug action) and let's define ExpRR as the relative risk between AAR and aaR, as

ExpRR=AAR/aaR.

From the previous equations, we have the following relationships:

ObsRR(ExpRR(1/PlaR

TntR/PlaR=(AAR*p2+AaR*2pq+aaR*q2)/PlaR

The maximum of the expected relative risk, max(ExpRR), corresponding to the case of heterozygous patients having the same response rate as the placebo rate, is such that:

$$ObsRR = {ExpRR}^{*} p2 + 2 pq + q2 ⇔ ExpRR = ( ObsRR - 2 pq - q2 ) / p2$$

The minimum of the expected relative risk, min(ExpRR), corresponding to the case of heterozygous patients having the same response rate as the homozygous non-affected patients, is such that:

$$ObsRR = {ExpRR}^{*} ( p2 + 2 pq ) + q2 ⇔ ExpRR = ( ObsRR - q2 ) / ( p2 + 2 pq )$$

For example, if q=0.4, PlaR=40% and ObsRR=1.5 (i.e. TntR=60%), then 1.6 (ExpRR (2.4. This means that the best treatment response rate we can expect in a genotypic subgroup of patients in these conditions would be 95.6% instead of 60%.

This can also be expressed in terms of maximum potential gain between the observed difference in response rates (TntR−PlaR) without any pharmacogenetic hypothesis and the maximum expected difference in response rates (max(ExpRR)*PlaR−TntR) with a strong pharmacogenetic hypothesis:

$$( max ( ExpRR ) * PlaR - TntR ) = [ ( ObsRR - 2 pq - q2 ) / p2 ] * PlaR - TntR ⇔ ( max ( ExpRR ) * PlaR - TntR ) = [ TntR - PlaR * ( 2 pq + q2 ) - Tntr * p2 ] / p2 ⇔ ( max ( ExpRR ) * PlaR - TntR ) = [ TntR * ( 1 - p2 ) - PlaR * ( 2 pq + q2 ) ] / p2 ⇔ ( max ( ExpRR ) * PlaR - TntR ) = [ ( 1 - p2 ) / p2 ] * ( TntR - PlaR )$$

that is for the previous example,

(95.6%−60%)=[(1−0.62)/0.62]*(60% −40%)=35.6%

Suppose that, instead of one SNP, we have p loci of SNPs for one gene. This means that we have 2p possible haplotypes for this gene and (2p)(2p−1)/2 possible genotypes. And with 2 genes with p1 and p2 SNP loci, we have [(2p1)(2p1−1)/2]*[(2p2)(2p2−1)/2] possibilities; and so on. Examining haplotypes instead of combinations of SNPs is especially useful when there is linkage disequilibrium enough to reduce the number of combinations to test, but not complete since in this latest case one SNP would be sufficient. Yet the problem of frequency above still remains with haplotypes instead of SNPs since the frequency of a haplotype cannot be higher than the highest SNP frequency involved. Hence cladograms.

Statistical Methods to be used in Objective Analyses

The statistical significance of the differences between variance frequencies can be assessed by a Pearson chi-squared test of homogeneity of proportions with n−1 degrees of freedom. Then, in order to determine which variance(s) is(are) responsible for an eventual significance, we can consider each variance individually against the rest, up to n comparisons, each based on a 2×2 table. This should result in chi-squared tests that are individually valid, but taking the most significant of these tests is a form of multiple testing. A Bonferroni's adjustment for multiple testing will thus be made to the P-values, such as p*=1−(1−p)ⁿ. Chi square on 3 genotypes, on haplotypes.

The statistical significance of the difference between genotype frequencies associated to every variance can be assessed by a Pearson chi-squared test of homogeneity of proportions with 2 degrees of freedom, using the same Bonferroni's adjustment as above.

Testing for unequal haplotype frequencies between cases and controls can be considered in the same framework as testing for unequal variance frequencies since a single variance can be considered as a haplotype of a single locus. The relevant likelihood ratio test compares a model where two separate sets of haplotype frequencies apply to the cases and controls, to one where the entire sample is characterized by a single common set of haplotype frequencies. This can be performed by repeated use of a computer program (Terwilliger and Ott, 1994, Handbook of Human Linkage Analysis, Baltimore, John Hopkins University Press) to successively obtain the log-likelihood corresponding to the set of haplotpe frequency estimates on the cases (1nL_{case}), on the controls (1nL_{control}), and on the overall (1nL_{combined}). The test statistic 2((1nL_{case})+(1nL_{control})−(1nL_{combined})) is then chi-squared with r−1 degrees of freedom (where r is the number of haplotypes).

To test for potentially confounding effects or effect-modifiers, such as sex, age, etc., logistic regression can be used with case-control status as the outcome variable, and genotypes and covariates (plus possible interactions) as predictor variables.

### EXAMPLE 11

### Exemplary Pharmacogenetic Analysis Steps

In accordance with the discussion of distribution frequencies for variances, alleles, and haplotypes, variance detection, and correlation of variances or haplotypes with treatment response variability, the points below list major items which will typically be performed in an analysis of the pharmacogenetic determination of the effects of variances in the treatment of a disease and the selection/optimization of treatment.

1) List candidate gene/genes for a known genetic disease, and assign them to the respective metabolic pathways.

2) Determine their alleles, observed and expected frequencies, and their relative distributions among various ethnic groups, gender, both in the control and in the study (case) groups.

3) Measure the relevant clinical/phenotypic (biochemical/physiological) variables of the disease.

4) If the causal variance/allele in the candidate gene is unknown, then determine linkage disequilibria among variances of the candidate gene(s).

5) Divide the regions of the candidate genes into regions of high linkage disequilibrium and low disequilibrium.

6) Develop haplotypes among variances that show strong linkage disequilibrium using the computation methods.

7) Determine the presence of rare haplotypes experimentally. Confirm if the computationally determined rare haplotypes agree with the experimentally determined haplotypes.

8) If there is a disagreement between the experimentally determined haplotypes and the computationally derived haplotypes, drop the computationally derived rare haplotypes, construct cladograms from these haplotypes using the Templeton (1987) algorithm.

9) Note regions of high recombination. Divide regions of high recombination further to see patterns of linkage disequilibria.

10) Establish association between cladograms and clinical variables using the nested analysis of variance as presented by Templeton (1995), and assign causal variance to a specific haplotype.

11) For variances in the regions of high recombination, use permutation tests for establishing associations between variances and the phenotypic variables.

12) If two or more genes are found to affect a clinical variable determine the relative contribution of each of the genes or variances in relation to the clinical variable, using step-wise regression or discriminant function or principal component analysis.

13) Determine the relative magnitudes of the effects of any of the two variances on the clinical variable due to their genetic (additive, dominant or epistasis) interaction.

14) Using the frequency of an allele or haplotypes, as well as biochemical/clinical variables determined in the in vitro or in vivo studies, determine the effect of that gene or allele on the expression of the clinical variable, according to the measured genotype approach of Boerwinkle et al (Ann. Hum. Genet 1986).

15) Stratify ethnic/clinical populations based on the presence or absence of a given allele or a haplotype.

16) Optimize drug dosages based on the frequency of alleles and haplotypes as well as their effects using the measured genotype approach as a guide.

### EXAMPLE 12

### Method for Producing cDNA

In order to identify sequence variances in a gene by laboratory methods it is in some instances useful to produce cDNA(s) from multiple human subjects. (hi other instances it may be preferable to study genomic DNA.). Methods for producing cDNA are known to those skilled in the art, as are methods for amplifying and sequencing the cDNA or portions thereof. An example of a useful cDNA production protocol is provided below. As recognized by those skilled in the art, other specific protocols can also be used.

cDNA Production

Make sure that all tubes and pipette tips are RNase-free. (Bake them overnight at 100° C. in a vaccum oven to make them RNase-free.)

1. Add the following to a RNase-free 0.2 ml micro-amp tube and mix gently:

24 ul water (DEPC treated)

12 ul RNA (lug/ul)

12 ul random hexamers (50 ng/ul)

2. Heat the mixture to 70° C. for ten minutes.

3. Incubate on ice for 1 minute.

4. Add the following:

16 ul 5×Synthesis Buffer

8ul 0.1M DTT

4 ul 10 mM dNTP mix (10 mM each dNTP)

4 ul SuperScript RT II enzyme

Pipette gently to mix.

5. Incubate at 42° C. for 50 minutes.

6. Heat to 70° C. for ten minutes to kill the enzyme, then place it on ice.

7. Add 160 ul of water to the reaction so that the final volume is 240 ul.

8. Use PCR to check the quality of the cDNA. Use primer pairs that will give a ˜800 base pair long piece. See “PCR Optimization” for the PCR protocol.

The following chart shows the reagent amounts for a 20 ul reaction, a 80 ul reaction, and a batch of 39 (which makes enough mix for 36) reactions:

| | 20 ul X 1 tube | 80 ul X 1 tube | 80 ul X 39 tubes |
|---|---|---|---|
| water | 6 ul | 24 ul | 936 |
| RNA | 3 ul | 12 ul | |
| random hexamers | 3 ul | 12 ul | 468 |
| synthesis buffer | 4 ul | 16 ul | 624 |
| 0.1 M DTT | 2 ul | 8 ul | 312 |
| 10 mM dNTP | 1 ul | 4 ul | 156 |
| SSRT | 1 ul | 4 ul | 156 |

### EXAMPLE 13

### Method for Detecting Variances by Single Strand Conformation Polymorphism (SSCP) Analysis

This example describes the SSCP technique for identification of sequence variances of genes. SSCP is usually paired with a DNA sequencing method, since the SSCP method does not provide the nucleotide identity of variances. One useful sequencing method, for example, is DNA cycle sequencing of ³²P labeled PCR products using the Femtomole DNA cycle sequencing kit from Promega (WI) and the instructions provided with the kit. Fragments are selected for DNA sequencing based on their behavior in the SSCP assay.

Single strand conformation polymorphism screening is a widely used technique for identifying an discriminating DNA fragments which differ from each other by as little as a single nucleotide. As originally developed by Orita et al. (Detection of polymorphisms of human DNA by gel electrophoresis as single-strand conformation polymorphisms. Proc Natl Acad Sci USA. 86(8):2766-70, 1989), the technique was used on genomic DNA, however the same group showed that the technique works very well on PCR amplified DNA as well. In the last 10 years the technique has been used in hundreds of published papers, and modifications of the technique have been described in dozens of papers. The enduring popularity of the technique is due to (1) a high degree of sensitivity to single base differences (>90%) (2) a high degree of selectivity, measured as a low frequency of false positives, and (3) technical ease. SSCP is almost always used together with DNA sequencing because SSCP does not directly provide the sequence basis of differential fragment mobility. The basic steps of the SSCP procedure are described below.

When the intent of SSCP screening is to identify a large number of gene variances it is useful to screen a relatively large number of individuals of different racial, ethnic and/or geographic origins. For example, 32 or 48 or 96 individuals is a convenient number to screen because gel electrophoresis apparatus are available with 96 wells (Applied Biosystems Division of Perkin Elmer Corporation), allowing 3×32, 2×48 or 96 samples to be loaded per gel.

The 32 (or more) individuals screened should be representative of most of the worlds major populations. For example, an equal distribution of Africans, Europeans and Asians constitutes a reasonable screening set. One useful source of cell lines from different populations is the Coriell Cell Repository (Camden, N.J.), which sells EBV immortalized lyphoblastoid cells obtained from several thousand subjects, and includes the racial/ethnic/geographic background of cell line donors in its catalog. Alternatively, a panel of cDNAs can be isolated from any specific target population.

SSCP can be used to analyze cDNAs or genomic DNAs. For many genes cDNA analysis is preferable because for many genes the full genomic sequence of the target gene is not available, however, this circumstance will change over the next few years. To produce cDNA requires RNA. Therefore each cell lines is grown to mass culture and RNA is isolated using an acid/phenol protocol, sold in kit form as Trizol by Life Technologies (Gaithersberg, Md.). The unfractionated RNA is used to produce cDNA by the action of a modified Maloney Murine Leukemia Virus Reverse Transcriptase, purchased in kit form from Life Technologies (Superscript II kit). The reverse transcriptase is primed with random hexamer primers to initiate cDNA synthesis along the whole length of the RNAs. This proved useful later in obtaining good PCR products from the 5′ ends of some genes. Alternatively, oligodT can be used to prime cDNA synthesis.

Material for SSCP analysis can be prepared by PCR amplification of the cDNA in the presence of one α ³²p labeled dNTP (usually α ³²p dCTP). Usually the concentration of nonradioactive dCTP is dropped from 200 uM (the standard concentration for each of the four dNTPs) to about 100 uM, and ³²p dCTP is added to a concentration of about 0.1-0.3 uM. This involves adding a 0.3-1 ul (3-10 uCi) of ³²P cCTP to a 10 ul PCR reaction. Radioactive nucleotides can be purchased from DuPont/New England Nuclear.

The customary practice is to amplify about 200 base pair PCR products for SSCP, however, an alternative approach is to amplify about 0.8-1.4 kb fragments and then use several cocktails of restriction endonucleases to digest those into smaller fragments of about 0.1-0.4 kb, aiming to have as many fragments as possible between 0.15 and 0.3 kb. The digestion strategy has the advantage that less PCR is required, reducing both time and costs. Also, several different restriction enzyme digests can be performed on each set of samples (for example 96 cDNAs), and then each of the digests can be run separately on SSCP gels. This redundant method (where each nucleotide is surveyed in three different fragments) reduces both the false negative and false positive rates. For example: a site of variance might lie within 2 bases of the end of a fragment in one digest, and as a result not affect the conformation of that strand; the same variance, in a second or third digest, would likely lie in a location more prone to affect strand folding, and therefore be detected by SSCP.

After digestion, the radiolabelled PCR products are diluted 1:5 by adding formamide load buffer (80% formamide, 1×SSCP gel buffer) and then denatured by heating to 90% C for 10 minutes, and then allowed to renature by quickly chilling on ice. This procedure (both the dilution and the quick chilling) promotes intra- (rather than inter-) strand association and secondary structure formation. The secondary structure of the single strands influences their mobility on nondenaturing gels, presumably by influencing the number of collisions between the molecule and the gel matrix (i.e., gel sieving). Even single base differences consistently produce changes in intrastrand folding sufficient to register as mobility differences on SSCP.

The single strands were then resolved on two gels, one a 5.5% acrylamide, 0.5×TBE gel, the other an 8% acrylamide, 10% glycerol, 1×TTE gel. (Other gel recipes are known to those skilled in the art.) The use of two gels provides a greater opportunity to recognize mobility differences. Both glycerol and acrylamide concentration have been shown to influence SSCP performance. By routinely analyzing three different digests under two gel conditions (effectively 6 conditions), and by looking at both strands under all 6 conditions, one can achieve a 12-fold sampling of each base pair of cDNA. However, if the goal is to rapidly survey many genes or cDNAs then a less redundant procedure would be optimal.

### EXAMPLE 14

### Method for Detecting Variances by T4 Endonuclease VII (T4E7) Mismatch Cleavage Method

The enzyme T4 endonuclease VII is derived from the bacteriophage T4. T4 endonuclease VII is used by the bacteriophage to cleave branched DNA intermediates which form during replication so the DNA can be processed and packaged. T4 endonuclease can also recognize and cleave heteroduplex DNA containing single base mismatches as well as deletions and insertions. This activity of the T4 endonuclease VII enzyme can be exploited to detect sequence variances present in the general population.

The following are the major steps involved in identifying sequence variations in a candidate gene by T4 endonuclease VII mismatch cleavage:

1. Amplification by the polymerase chain reaction (PCR) of 400-600 bp regions of the candidate gene from a panel of DNA samples The DNA samples can either be cDNA or genomic DNA and will represent some cross section of the world population.

2. Mixing of a fluorescently labeled probe DNA with the sample DNA.

Heating and cooling the mixtures causing heteroduplex formation between the probe DNA and the sample DNA.

3. Addition of T4 endonuclease VII to the heteroduplex DNA samples. T4 endonuclease will recognize and cleave at sequence variance mismatches formed in the heteroduplex DNA.

4. Electrophoresis of the cleaved fragments on an ABI sequencer to determine the site of cleavage.

5. Sequencing of a subset of PCR fragments identified by T4 endonuclease VI to contain variances to establish the specific base variation at that location.

A more detailed description of the procedure is as follows:

A candidate gene sequence is downloaded from an appropriate database. Primers for PCR amplification are designed which will result in the target sequence being divided into amplification products of between 400 and 600 bp. There will be a minimum of a 50 bp of overlap not including the primer sequences between the 5′ and 3′ ends of adjacent fragments to ensure the detection of variances which are located close to one of the primers.

Optimal PCR conditions for each of the primer pairs is determined experimentally. Parameters including but not limited to annealing temperature, pH, MgCl₂ concentration, and KCl concentration will be varied until conditions for optimal PCR amplification are established. The PCR conditions derived for each primer pair is then used to amplify a panel of DNA samples (cDNA or genomic DNA) which is chosen to best represent the various ethnic backgrounds of the world population or some designated subset of that population.

One of the DNA samples is chosen to be used as a probe. The same PCR conditions used to amplify the panel are used to amplify the probe DNA. However, a flourescently labeled nucleotide is included in the deoxy-nucleotide mix so that a percentage of the incorporated nucleotides will be fluorescently labeled.

The labeled probe is mixed with the corresponding PCR products from each of the DNA samples and then heated and cooled rapidly. This allows the formation of heteroduplexes between the probe and the PCR fragments from each of the DNA samples. T4 endonuclease VII is added directly to these reactions and allowed to incubate for 30 min. at 37 C. 10 ul of the Formamide loading buffer is added directly to each of the samples and then denatured by heating and cooling. A portion of each of these samples is electrophoresed on an ABI 377 sequencer. If there is a sequence variance between the probe DNA and the sample DNA a mismatch will be present in the heteroduplex fragment formed. The enzyme T4 endonuclease VII will recognize the mismatch and cleave at the site of the mismatch. This will result in the appearance of two peaks corresponding to the two cleavage products when run on the ABI 377 sequencer.

Fragments identified as containing sequencing variances are subsequently sequenced using conventional methods to establish the exact location and sequence variance.

### EXAMPLE 15

### Method for Detecting Variances by DNA Sequencing

Sequencing by the Sanger dideoxy method or the Maxim Gilbert chemical cleavage method is widely used to determine the nucleotide sequence of genes.

Presently, a worldwide effort is being put forward to sequence the entire human genome. The Human Genome Project as it is called has already resulted in the identification and sequencing of many new human genes. Sequencing can not only be used to identify new genes, but can also be used to identify variations between individuals in the sequence of those genes.

The following are the major steps involved in identifying sequence variations in a candidate gene by sequencing:

1. Amplification by the polymerase chain reaction (PCR) of 400-700 bp regions of the candidate gene from a panel of DNA samples The DNA samples can either be cDNA or genomic DNA and will represent some cross section of the world population.

2. Sequencing of the resulting PCR fragments using the Sanger dideoxy method. Sequencing reactions are performed using flourescently labeled dideoxy terminators and fragments are separated by electrophoresis on an ABI 377 sequencer or its equivalent.

3. Analysis of the resulting data from the ABI 377 sequencer using software programs designed to identify sequence variations between the different samples analyzed.

A more detailed description of the procedure is as follows:

A candidate gene sequence is downloaded from an appropriate database.

Primers for PCR amplification are designed which will result in the target sequence being divided into amplification products of between 400 and 700 bp. There will be a minimum of a 50 bp of overlap not including the primer sequences between the 5′ and 3′ ends of adjacent fragments to ensure the detection of variances which are located close to one of the primers.

Optimal PCR conditions for each of the primer pairs is determined experimentally. Parameters including but not limited to annealing temperature, pH, MgCl₂ concentration, and KCl concentration will be varied until conditions for optimal PCR amplification are established. The PCR conditions derived for each primer pair is then used to amplify a panel of DNA samples (cDNA or genomic DNA) which is chosen to best represent the various ethnic backgrounds of the world population or some designated subset of that population.

PCR reactions are purified using the QIAquick 8 PCR purification kit (Qiagen cat# 28142) to remove nucleotides, proteins and buffers. The PCR reactions are mixed with 5 volumes of Buffer PB and applied to the wells of the QlAquick strips. The liquid is pulled through the strips by applying a vacuum. The wells are then washed two times with 1 ml of buffer PE and allowed to dry for 5 minutes under vacuum. The PCR products are eluted from the strips using 60 ul of elution buffer.

The purified PCR fragments are sequenced in both directions using the Perkin Elmer ABI Prism™ Big Dye™ terminator Cycle Sequencing Ready Reaction Kit (Cat# 4303150). The following sequencing reaction is set up: 8.0 ul Terminator Ready Reaction Mix, 6.0 ul of purified PCR fragment, 20 picomoles of primer, deionized water to 20 ul. The reactions are run through the following cycles 25 times: 96° C. for 10 second, annealing temperature for that particular PCR product for 5 seconds, 60° C. for 4 minutes.

The above sequencing reactions are ethanol precipitated directly in the PCR plate, washed with 70% ethanol, and brought up in a volume of 6 ul of formamide dye. The reactions are heated to 90° C. for 2 minutes and then quickly cooled to 4° C. 1 ul of each sequencing reaction is then loaded and run on an ABI 377 sequencer.

The output for the ABI sequencer appears as a series of peaks where each of the different nucleotides, A, C, G, and T appear as a different color. The nucleotide at each position in the sequence is determined by the most prominent peak at each location. Comparison of each of the sequencing outputs for each sample can be examined using software programs to determine the presence of a variance in the sequence. One example of heterozygote detection using sequencing with dye labeled terminators is described by Kwok et. al (Kwok, P. -Y.; Carlson, C.; Yager, T. D., Ankener, W., and D. A. Nickerson, Genomics 23, 138-144, 1994). The software compares each of the normalized peaks between all the samples base by base and looks for a 40% decrease in peak height and the concomitant appearance of a new peak underneath. Possible variances flagged by the software are further analyzed visually to confirm their validity.

### EXAMPLE 16

### Exemplary Pharmacogenetic Analysis Steps—Biological Function Analysis

In many cases when a gene which may affect drug action is found to exhibit variances in the gene, RNA, or protein sequence, it is preferable to perform biological experiments to determine the biological impact of the variances on the structure and function of the gene or its expressed product and on drug action. Such experiments may be performed in vitro or in vivo using methods known in the art.

The points below list major items which may typically be performed in an analysis of the effects of variances in the treatment of a disease and the selection/optimization of treatment using biological studies to determine the structure and function of variant forms of a gene or its expressed product.

1) List candidate gene/genes for a known genetic disease, and assign them to the respective metabolic pathways.

2) Identify variances in the gene sequence, the expressed mRNA sequence or expressed protein sequence.

3) Match the position of variances to regions of the gene, mRNA, or protein with known biological functions. For example, specific sequences in the promotor of a gene are known to be responsible for determining the level of expression of the gene; specific sequences in the mRNA are known to be involved in the processing of nuclear mRNA into cytoplasmic mRNA including splicing and polyadenylation; and certain sequences in proteins are known to direct the trafficking of proteins to specific locations within a cell and to constitute active sites of biological functions including the binding of proteins to other biological consituents or catalytic functions. Variances in sites such as these, and others known in the art, are candidates for biological effects on drug action.

4) Model the effect of the variance on mRNA or protein structure. Computational methods for predicting the structure of mRNA are known and can be used to assess whether a specific variance is likely to cause a substantial change in the structure of mRNA. Computational methods can also be used to predict the structure of peptide sequences enabling predictions to be made concerning the potential impact of the variance on protein function. Most useful are structures of proteins determined by X-ray diffraction, NMR or other methods known in the art which provide the atomic structure of the protein. Computational methods can be used to consider the effect of changing an amino acid within such a structure to determine whether such a change would disrupt the structure and/or function of the protein. Those skilled in the art will recognize that this analysis can be performed on crystal structures of the protein known to have a variance as well as homologous proteins expressed from different loci in the human genome, or homologous proteins from other species, or non-homologous but analogous proteins with similar functions from humans or other species.

5) Produce the gene, mRNA or protein in amounts sufficient to experimentally characterize the structure and function of the gene, mRNA or protein. It will be apparent to those skilled in the art that by comparing the activity of two genes or their products which differ by a single variance, the effect of the variance can be determined. Methods for producing genes or gene products which differ by one or more bases for the purpose of experimental analysis are known in the art.

6) Experimental methods known in the art can be used to determine whether a specific variance alters the transcription of a gene and translation into a gene product. This involves producing amounts of the gene by molecular cloning sufficient for in vitro or in vivo studies. Methods for producing genes and gene products are known in the art and include cloning of segments of genetic material in prokaryotes or eukarotic hosts, run off transcription and cell-free translation assays that can be performed in cell free extracts, transfection of DNA into cultured cells, introduction of genes into live animals or embryos by direct injection or using vehicles for gene delivery including transfection mixtures or viral vectors.

7) Experimental methods known in the art can be used to determine whether a specific variance alters the ability of a gene to be transcribed into RNA. For example, run off transcription assays can be performed in vitro or expression can be characterized in transfected cells or transgenic animals.

8) Experimental methods known in the art can be used to determine whether a specific variance alters the processing, stability, or translation of RNA into protein. For example, reticulocyte lysate assays can be used to study the production of protein in cell free systems, transfection assays can be designed to study the production of protein in cultured cells, and the production of gene products can be measured in transgenic animals.

9) Experimental methods known in the art can be used to determine whether a specific variant alters the activity of an expressed protein product. For example, protein can be producted by reticulocyte lystae systems or by introducing the gene into prokaryotic organisms such as bacteria or lowre eukaryotic organisms such as yeast or fungus), or by introducing the gene into cultured cells or transgenic animals. Protein produced in such systems can be extracted or purified and subjected to bioassays known to those in the art as measures of the nction of that particular protein. Bioassays may involve, but are not limited to, binding, inhibiton, or catalytic functions.

10) Those skilled in the art will recognize that it is sometimes preferred to perform the above experiments in the presence of a specific drug to determine whether the drug has differential effects on the activity being measured. Alternatively, studies may be performed in the presence of an analogue or metabolite of the drug.

11) Using methods described above, specific variances which alter the biological function of a gene or its gene product that could have an impact on drug action can be identified. Such variances are then studied in clinical trial populations to determine whether the presence or absence of a specific variance correlates with observed clinical outcomes such as efficacy or toxicity.

12) It will be further recognized that there may be more than one variance within a gene that is capable of altering the biological function of the gene or gene product. These variances may exhibit similar, synergistic effects, or may have opposite effects on gene function. In such cases, it is necessary to consider the haplotype of the gene, namely the combination of variances that are present within a single allele, to assess the composite function of the gene or gene product.

13) Perform clinical trials with stratification of patients based on presence or absence of a given variance, allele or haplotype of a gene. Establish associations between observed drug responses such as toxicity, efficacy, drug response, or dose toleration and the presence or absense of a specific variance, allele, or haplotype.

14) Optimize drug dosage or drug usage based on the presence of the variant.

### EXAMPLE 17

### Stratification of Patients by Genotype in Prospective Clinical Trials

In a prospective clinical trial, patients will be stratified by genotype to determine whether the observed outcomes are different in patients having different genotypes. A critical issue is the design of such trials to assure that a sufficient number of patients are studied to observe genetic effects.

The number of patients required to achieve statistical significance in a conventional clinical trial is calculated from:

N=2(z_{α}+z_{2β})²/(δ/σ)² (two tailed test) 1.1

From this equation it may be inferred that the size of a genetically defined subgroup Nᵢ required to achieve statistical significance for an observed outcome associated with variance or haplotype “i” can be calculated as:

Nᵢ=2(z_{α}+z_{2β})²/(δᵢ/σᵢ)² 1.2

If Pᵢ is the prevalence of the genotype “i” in the population, the total number of patients that need to be incorporated in a clinical trial N_{g} to identify a population with haplotype “i” of size Nᵢ is given by:

N_{g}=Nᵢ/Pᵢ 1.3

It should be noted that N_{g} describes the total number of patients that need to be genotyped in order to identify a subset of Nᵢ patients with genotype “i”.

If genotyping is used as means for statistical stratification of patients, N_{g} represents the number of patients that would need to be enrolled in a trial to achieve statistical significance for subgroup “i”. If genotyping is used as a means for inclusion, it represents the number of patients that need to screened to identify a population of Nᵢ individuals for an appropriately powered clinical trial. Thus, N_{g} is a critical determinant of the scope of the clinical trial as well as Nᵢ.

A clinical trial can also be designed to test associations for multiple genetic subgroups “j” defined by a single allele in which case:

N_{g}=max(N_{g1}) for i=1 . . . j 1.4

If more than one subgroup is tested, but there is no overlap in the patients contained within the subgroups, these can be considered to be independent hypotheses and no multiple testing correction should be required. If consideration of more than one subgroup constitutes multiple testing, or if individual patients are included in multiple subgroups, then statistical corrections may required in the values of z_{α} or z_{2β} which would increase the number of patients required.

It should be emphasized that a clinical trial of this nature may not provide statistically significant data concerning associations with any genotype other than “i”. The total number of patients that would be required in a clinical trial to test more than one genetically defined subgroup would be determined by the maximum value of N_{g} for any single subgroup.

The power of pharmacogenomics to improve the efficiency of clinical trials arises from the fact it is possible to have N_{g}<N. The goal of pharmacogenomic analysis is to identify a genetically define subgroup in which the magnitude of the clinical response is greater and the variability in response is reduced. These observations correspond to an increase in the magnitude of the (mean) observed response δ or a decrease the degree of variability σ. Since the value of Nᵢ calculated in equation 1.2 decreases non-linearly as the square of these changes, the total number of patients N_{g} can also decrease non-linearly, resulting in a clinical trial that requires fewer patients to achieve statistical significance. If δ₁ and σ₁ are not different than δ and σ, then N_{g} is greater than N as given by N_{g}=N₁/P₁. Values of δ₁ and σ₁ that give N_{g}<N can be calculated:

N_{g}<N if: Pᵢ>[(δ/σ)²]/[(δᵢ/σ₁)²] 1.5

It is apparent from this analysis that N_{g} is not uniformly less than N, even with modest improvements in the values for δᵢ and σᵢ.

As with a conventional clinical trial, the incorporation of an appropriate control group in the study design is critical for achieving success. In the case of a prospective clinical trial, the control group commonly is selected on the basis of the same inclusion criteria as the treatment group, but is treated with placebo or a standard therapeutic regimen rather than the investigational drug. In the case of a study with subgroups that are defined by haplotype, the ideal control group for a treatment subgroup with hapotype “i” is a placebo-treated subgroup with haplotype “i”. This is often a critical control, since haplotypes which may be associated with the response to treatment may also affect the natural course of the disease.

A critical issue in considering control groups is that σ for the control group placebo treated population with haplotype “i” may not be equivalent to that of the control population. If so, 1.5 may overestimate the benefits of any reduction in σᵢ in the treatment response group if there is not also a reduction in σᵢ in the control group.

If σ of the treatment and control groups are not equivalent, δ would be still calculated as the difference in the response of the two groups, but σ would be different in the two groups with values of σ₀ or σ₁ respectively. In this case, the number of patients in the genetically defined subgroup N₁ would be defined by:

Nᵢ=(σZ_{α}+σᵢZ_{β})²/δ² 2.1

The total number of patients that would need to be enrolled in such a trial would be the maximium of

N or N/Pi 2.2

It will be apparent that such an analysis remains sensitive to increases in δ, but is less sensitive to changes in σ which are not also reflected in the control group.

Certain analysis may be performed by comparing individuals with one haplotype against the entire normal population. Such an analysis may be used to establish the selectivity of the response associated with a specific haplotype. For example, it may be desirable to establish that the response or toxicity observed in a specific subgroup is greater than that associated observed with the entire population. It may also be of interest to compare the response to treatment between two different subgroups. If σ differs between the groups, then the estimate of the number of patients that need to be enrolled in the trial must be calculated using equations 2.1 with N being the maximum of N₁/P₁ for the different subgroups.

Another issue in controls is the relative size of the treatment and control groups. In a prospectively designed clinical trial which selectively incorporates patients with haplotype “i” the number of patients in the control and treatment group will be essentially equivalent. If the control group is different, or if haplotypes are used for stratification but not inclusion, statistical corrections may need to be made for having populations of different size.

### EXAMPLE 18

### Stratification of Patients by Phenotype

The identification of genetic associations in Phase II or retrospective studies can be performed by stratifying patients by phenotype and analyzing the distribution of genotypes/haplotypes in the separate populations. A particularly important aspect of this analysis is that any gene may have only a partial effect on the observed outcome, meaning that there will be an association value (A) corresponding to the fraction of patients in a phenotypically-defined subgroup who exhibit that phenotype due to a specific genotype/phenotype.

It will be recognized to those skilled in the art that the fraction of individuals who exhibit a phenotype due to any specific allele will be less than 1 (i.e. A<1). This is true for several reasons. The observed phenotype may occur by random chance. The observed phenotype may be associated with environmental influences, or the observed phenotype may be due to different genetic effects in different individuals. Furthermore, the onstruction of haplotypes and analysis of recombination may not group all alleles with pheontypically-significant variances within a single haplotype or haplotype cluster. In this case, causative variances at a single locus may be associated with more than one haplotype or haplotype cluster and the association constant A for the locus would be A=A₁+A₂+. . . +Aₙ<1. It is likely that many phenotypes will be associated with multiple alleles at a given locus, and it is particularly important that statistical methods be sufficiently robust to identify association with a locus even if Aᵢ is reduced by the presence of several causative alleles.

Statistical methods can be used to identify genetic effects on an observed outcome in patient groups stratified by phenotype, eg the presence or absence of the observed response. One such method entails determining the allele frequencies in two populations of patients stratified by an observed clinical outcome, for example efficacy or toxicity and performing a maximum likelihood analysis for the association between a given gene and the observed phenotype based on the allele frequencies and a range of values for A (the association constant between a specific allele and the observed outcome used to stratify patients).

This analysis is performed by comparing the observed gene frequencies in a patient population with an observed outcome to gene frequencies in a table in which the predicted frequencies of different alleles of the gene assuming different values of the association constant A for that allele. This table of predicted gene frequencies can be constructed by those skilled in the art based on the frequency of any specific allele in the normal population, the predicted inheritance of the effect (e.g. dominant or recessive) and the fraction of a subgroup with a specific outcome who would have that allele based on the association constant A.

For example, if a specific outcome was only observed in the presence of a specific allele of a gene, the expected frequency would be 1. If a specific outcome was never observed in the presence of a specific allele of a gene, the expected fequency would be 0. If there was no association between the allele and the observed outcome, the frequency of that allele among individuals with an observed outcome would be the same as in the general population. A statistical analysis can be performed to compare the observed allele frequencies with the predicted allele frequencies and determine the best fit or maximum likeihood of the association. For example, a chi square analysis will determine whether the observed outcome is statistically similar to predicted outcomes calculated for different modes of inheritance and different potential values of A. P values can then be calculated to determine the likelihood that any specific association is statistically significant. A curve can be calculated based on different values of A, and the maximal likelihood of an association determined from the peak of such a curve. Methods for chi square analysis are known to those in the art.

A multidimensional analysis can also be performed to determine whether an observed outcome is associated with more than one allele at a specific genetic locus. An example of this analysis considering the potential effects of two different alleles of a single gene is shown. It will be apparent to those skilled in the art that this analysis can be extended to n dimensions using computer programs.

This analysis can be used to determine the maximum likelihood that one or more alleles at a given locus are associated with a specific clinical outcome.

It will be apparent to those skilled in the art that critical issues in this analysis include the fidelity of the phenotypic association and identification of a control group. In particular, it may be useful to perform an identical analysis in patients receiving a placebo to eliminate other forms of bias which may contribute to statistical errors.

### Other Embodiments

The invention described herein provides a method for identifying patients with a risk of developing drug-induced liver disease or hepatic dysfunction by determining the patients allele status for a gene listed in Tables 1 and 2 and providing a forecast of the patients ability to respond to a given drug treatment. In particular, the invention provides a method for determining, based on the presence or absence of a polymorphism, a patient's likely response to drug therapies as drug-induced liver disease or hepatic dysfunction. Given the predictive value of the described polymorphisms across two different classes of drug, having different mechanisms of action, the candidate polymorphism is likely to have a similar predictive value for other drugs acting through other pharmacological mechanisms. Thus, the methods of the invention may be used to determine a patient's response to other drugs including, without limitation, antihypertensives, anti-obesity, anti-hyperlipidemic, or anti-proliferative, antioxidants, or enhancers of terminal differentiation.

In addition, while determining the presence or absence of the candidate allele is a clear predictor determining the efficacy of a drug on a given patient, other allelic variants of reduced catalytic activity are envisioned as predicting drug efficacy using the methods described herein. In particular, the methods of the invention may be used to treat patients with any of the possible variances, e.g., as described in Table 3 of Stanton & Adams, application Ser. No. 09/300,747, supra.

In addition, while the methods described herein are preferably used for the treatment of human patient, non-human animals (e.g., pets and livestock) may also be treated using the methods of the invention.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, using other compounds, and/or methods of administration are all within the scope of the present invention. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

TABLE 1

| Class | Pathway | Function | Name | OMIM | GID | Locus |
|---|---|---|---|---|---|---|
| Absorption | Gastrointestinal | Glycosidases | sucrase-isomaltase/S1 | 222900 | NM_001041 | 3q25-q26 |
| and | Drug Metabolism | | maltase-glucoamylase/alpha-glucosidase/MGAM | 154360 | NM_004668 | Chr.7 |
| Distribution | | | lactase-phlorizin hydrolase/LPH/lactase/LCT | 603202 | NM_002299 | 2q21 |
| | | | salivary amylase A/AMY1A | 104700 | NM_004038 | 1p21 |
| | | | salivary amylase B/AMY1B | 104701 | ****** | 1p21 |
| | | | salivary amylase C/AMY1C | 104702 | ****** | 1p22 |
| | | | pancreatic amylase A/AMY2A | 104650 | X07057 | 1p21 |
| | | | pancreatic amylase B/AMY2B | 104660 | ****** | 1p21 |
| | | Proteases and | dipeptidylpeptidase IV/CD26/ADA complexing | 102720 | NM_001935 | 2q23 |
| | | Peptidases | protein 2/DPP4 | | | |
| | | | pepsinogen A/PGA/PG | 169700 | AH001519 | 11q13 |
| | | | pepsinogen, group 3/PGA3 | 169710 | ****** | 11q13 |
| | | | | 169740 | J04443 | 6p21.3- |
| | | | pepsinogen C/PGC | | | q21.1 |
| | | | | 147910 | AH002853 | 19q13.2- |
| | | | | | | q13.4 |
| | | | chymotrypsin-like protease | 118888 | X71875 | 16q22.1 |
| | | | trypsinogen 1/TRY1/protease, serine 1/PRSS1 | 276000 | NM_002769 | 7q35 |
| | | | trypsinogen 1/TRY2/protease, serine 2/PRSS2 | 601564 | NM_002770 | 7q35 |
| | | | trypsinogen 1/TRY3/protease, serine 3/PRSS3 | ****** | NM_002771 | ****** |
| | | | enterokinase 1/TRY3/protease, serine7/PRSS7 | 226200 | NM_002772 | 21q21 |
| | | | chymotrypsinogen 1/CTRB1 | 118890 | NM_001906 | 16q23.2- |
| | | | | | | q23.3 |
| | | | carboxypeptidase A1/CPA1 | 114850 | NM_001868 | 7q32-qter |
| | | | carboxypeptidase A2/CPA2 | 600688 | NM_001869 | ****** |
| | | | carboxypeptidase Z/CPZ | 603105 | NM_003652 | |
| | | | elastase 1/ELA1 | 130120 | D00158 | 12q13 |
| | | | renal microsomal dipeptidase/DPEP1 (b-lactam ring | 179780 | NM_004413 16q24.3 | |
| | | | hydrolysis) | | | |
| | | | tripeptidyl peptidase II/TPP2 | 190470 | NM_003291 | 13q32- |
| | | | | | | q33 |
| | | | protease inhibitor 1/alpha-1-antitrypsin/AAT/PI | 107400 | NM_000295 | 14q32.1 |
| | | | protease inhibitor/alpha-1-antichymotrypsin/AACT | 107280 | NM_001085 | 14q32.1 |
| | | | protease inhibitor 1 (alpha-1-antitrypsin)-like/PIL | 107410 | NM_006220 | 14q32.1 |
| | | Lipases | Carboxyl ester lipase (bile salt-stimulated | 114840 | M85201 | 9q34.3 |
| | | | lipase)/CEL | | | |
| | | | Carboxyl ester lipase-like (bile salt-stimulated lipase- | 114841 | NM_001808 | 9q34.3 |
| | | | like)/CELL | | | |
| | | | Pancreatic colipase/CLPS | 120105 | M95529 | 6pter- |
| | | | | | | p21.1 |
| | | | Pancreatic triglyceride lipase/PNLTP | 246600 | AH003527 | 10q26.1 |
| | | | Lipoprotein lipase/LPL | 238600 | NM_000237 | 8p22 |
| | | | Hepatic triglyceride lipase/LIPC | 151670 | AH005429 | |
| | | Oxidases | salivary peroxidase/SAPX | 170990 | U39573 | ****** |
| | | | alcohol dehydrogenases 6/ADH6 | 103735 | NM_0006721 | 15q26 |
| | | Esterases | paraoxonase 2/PON2 | 602447 | L48513 | 7q21.3 |
| | | Phosphatases | intestinal alkaline phosphatase/ALPI | 171740 | NM_00163 | 12q36.3- |
| | | | | | | q37.1 |
| | | | tissue non-specific alkaline phosphatase/liver | 171760 | | 1p36.1- |
| | | | alkaline phosphatase/ALPL | | NM_000478 | p34 |
| | Drug Binding | Blood Transport | serum albumin/ALB | 103600 | NM_000477 | 4q11-q13 |
| | | | alpha fetoprotein/AFP | 104150 | NM_001134 | 4q11-g13 |
| | | | alpha albumin/afamin/AFM/ALB2 | 104145 | NM_001133 | 4q11-q13 |
| | | | vitamin D-binding protein/group-specific | 139200 | AH004448 | 4q12 |
| | | | component/GC | | | |
| | | | orosomucoid 1/alpha 1 acid glycoprotein/ORM1 | 138600 | M13692 | 9q34.1- |
| | | | | | | q34.3 |
| | | | orosomucoid 2/alpha 1 acid glycoprotein, type | 138610 | NM_000608 | 9q34.1- |
| | | | 2/ORM2 | | | q34.3 |
| | | | transthyretin (prealbumin, amyloidosis type I)/TTR | 176300 | NM_000371 | 18q11.2- |
| | | | | | | q12.1 |
| | | | thyroxin-binding globulin/TBG | 314200 | NM_000354 | Xq22.2 |
| | | | corticosteroid binding globulin precursor/CBG | 122500 | NM_001756 | 14q32.1 |
| | | | sex hormone-binding globulin/SHBG | 182205 | X16349 | 17p13- |
| | | | | | | p12 |
| | | | mannose-binding lectin, soluble/MBL2 | 154545 | NM_000242 | 10q11.2- |
| | | | | | | q21 |
| | | Bile Acid | Hepatic fatty acid binding protein/FABP1 | 134650 | ****** | 2p11 |
| | | Binders | Intestinal fatty acid binding protein/FABP2 | 134640 | NM_000134 | 4q28-q31 |
| | | | Muscle fatty acid binding protein/mammary-derived | 134651 | NM_004102 | 1p33-p31 |
| | | | growth inhibitor/MDGI/FABP3 | | | |
| | | | Adipocyte fatty acid binding protein/FABP4 | 600434 | NM_001442 | 8q21 |
| | | | Ileal fatty acid binding protein/FABP6 | 600422 | U19869 | 5q23-q35 |
| | | | Brain fatty acid binding protein/FABP7 | 602965 | D88648 | 6q22-q23 |
| | | | Adipocyte long chain fatty acid transport | 600691 | ****** | ****** |
| | | | protein/FATP | | | |
| | Drug Uptake | ABC | Retina-specific ATP binding cassette | 601691 | NM_000350 | 1p21-p13 |
| | | Transporters | transporter/ABCR | | | |
| | | | ATP binding cassette 1/ABC1 | 600046 | AJ012376 | 9q22-q31 |
| | | | ATP binding cassette 2/ABC2 | 600047 | U18235 | 9q34 |
| | | | ATP binding cassette 3/ABC3 | 601615 | NM_001089 | 16p13.3 |
| | | | ATP binding cassette 7/ABC7 | 300135 | AB005289 | Xq13.1- |
| | | | | | | q13.3 |
| | | | ATP binding cassette 8/ABC8 | 603076 | AF0381752 | 1q22.3 |
| | | | ATP-binding cassette 50/ABC50 | 603429 | AF0273026 | p21.33 |
| | | | Placenta-specific ATP-binding cassette | 603756 | NM_004827 | 4q22 |
| | | | transporter/ABCP | | | |
| | | | cystic fibrosis transmembrane conductance | 602421 | NM_000492 | 7q31.2 |
| | | | regulator/CFTR | | | |
| | | | adrenoleukodystrophy/adrenomyeloneuropathy/ALD | 300100 | NM_000033 | Xq28 |
| | | | adrenoleukodystrophy related protein/ALDR | 601081 | U28150 | 12q11- |
| | | | | | | q12 |
| | | | sulfonylurca receptor (hyperinsulinemia)/SUR | 600509 | NM_000352 | 11p15.1 |
| | | | peroxisomal membrane protein 1/PXMP1 | 170995 | NM_002858 | 1p22-p21 |
| | | | peroxisomal membrane protein 1-like/PXMP1L | 603214 | NM_005050 | 14q24.3 |
| | | | antigen peptide transporter 1/MHC 1/TAP1 | 170260 | NM_000593 | 6p21.3 |
| | | | antigen peptide transporter 2/MHC 2/TAP2 | 170261 | NM_000544 | 6p21.3 |
| | | | multidrug resistance associated protein MRP1 | 158343 | L05628 | 16p13.1 |
| | | | multidrug resistance associated protein | 601107 | NM_000392 | 10q24 |
| | | | MRP2/CMOAT | | | |
| | | | ATP-binding cassette, sub-family C (CFTR/MRP), | ****** | | ****** |
| | | | member 3/CMOAT2 | | NM_003786 | |
| | | | ATP-binding cassette, sub-family C (CFTR/MRP), | ****** | NM_005845 | ****** |
| | | | member 4/MOATB | | | |
| | | | ATP-binding cassette, sub-family C (CFTR/MRP), | ****** | NM_005688 | ****** |
| | | | member 5/SMRP | | | |
| | | | ATP-binding cassette, sub-family C (CFTR/MRP), | 601439 | NM_005691 | ****** |
| | | | member 9/SUR2 | | | |
| | | | multidrug resistance protein MDR1 | 171050 | X96395 | 7q21.1 |
| | | | multidrug resistance protein MDR3/P-glycoprotein | 602347 | X06181 | 7q21.1 |
| | | | 3/PGY3 | | | |
| | | | anthracyleline resistance-related protein/ARA | 603234 | NM_001171 | 16p13.1 |
| | | | bile salt export pump/BSEP | 603201 | NM_003742 | 2q24 |
| | | | familial intrahepatic cholestasis 1/FIC1 | 602397 | NM_005603 | 18q21 |
| | | | Human sorcin/SRI | 182520 | L12387 | 7q21.1 |
| | | Solute | Solute carrier family 1, member 1/SLC1A1 | 133550 | U08989 | 9p24 |
| | | Antiporters | (glutamate) | | | |
| | | | Solute carrier family 1, member 2/SLC1A2 | 600300 | U03505 | 11p13- |
| | | | (glutamate) | | | p12 |
| | | | Solute carrier family 1, member 3/SLC1A3 | 600111 | U03504 | 5p13 |
| | | | (glutamate) | | | |
| | | | Solute carrier family 1, member 4/SLC1A4 | 600229 | NM_003038 | 2p15-p13 |
| | | | (glutamate) | | | |
| | | | Solute carrier family 1, member 5/SLC1A5 (neutral | 109190 | AF105230 | 19q13.3 |
| | | | AA) | | | |
| | | | Solute carrier family 1, member 6/SLC1A6 | 600637 | NM_005071 | ****** |
| | | | (glutamate) | | | |
| | | | Solute carrier family 2, member 1/SLC2A1/SGLT1 | 182380 | NM_006516 | 22q13.1 |
| | | | (glucose) | | | |
| | | | Solute carrier family 2, member 2/SLC2A2/GLUT2 | 138160 | NM_006516 | 3q26.1- |
| | | | (glucose) | | | q26.3 |
| | | | Solute carrier family 2, member 3/SLC2A3/GLUT3 | 138170 | M20681 | 12p13.3 |
| | | | (glucose) | | | |
| | | | Solute carrier family 2, member 4/SLC2A4/GLUT4 | 138190 | ****** | 17p13 |
| | | | (glucose) | | | |
| | | | Solute carrier family 2, member 5/SLC2A5/GLUT5 | 138230 | NM_003039 | 1p36.2 |
| | | | (glucose) | | | |
| | | | Solute carrier family 3 member 1/SLC3A1 (aa | 104614 | ****** | 2p16.3 |
| | | | transporter) | | | |
| | | | Solute carrier family 5 member 1/SLC5A2 (glucose) | 182381 | ****** | 16p11.2 |
| | | | Solute carrier family 5 member 3/SLC5A3 | 600444 | L38500 | 21q22 |
| | | | Solute carrier family 5 member 6/SLC5A6 (folate, | 604024 | ****** | 2p23 |
| | | | biotin, lipoate) | | | |
| | | | Solute carrier family 6 member 1/SLC6A1 (GABA) | 137165 | X54673 | 3p25-p24 |
| | | | Solute carrier family 6 member 2/SLC6A2 | 163970 | NM_001043 | 16q12.2 |
| | | | (noradrenalin) | | | |
| | | | Solute carrier family 6 member 3/SLC6A3 | 126455 | L24178 | 5p15.3 |
| | | | (dopamine) | | | |
| | | | Solute carrier family 6 member 4/SLC6A4 | 182138 | X70697 | 17q11.1- |
| | | | (serotonin) | | | q12 |
| | | | Solute carrier family 6, member 5/SLC6A5 (glycine) | 604159 | NM_004211 | ****** |
| | | | Solute carrier family 6, member 6/SLC6A6 (taurine) | 186854 | U16120 | 3p25-q24 |
| | | | Solute carrier family 6, member 8/SLC6A8 (creatine) | 300036 | NM_005629 | 300036 |
| | | | Solute carrier family 6, member 9/SLC6A9 (glycine) | 601019 | S70612 | 1p313 |
| | | | Solute carrier family 6, member 10/SLC6A10 | 601294 | ****** | 16p11.2 |
| | | | (creatine-testis) | | | |
| | | | Solute carrier family 6, member 12/SLC6A12 | 603080 | NM_003044 | 12p13 |
| | | | (GABA-betaine) | | | |
| | | | Solute carrier family 7, member 1/SLC7A1 (cationic | 104615 | ****** | 13q12.3 |
| | | | AA) | | | |
| | | | Solute carrier family 7, member 2/SLC7A2 (cationic | 601872 | D29990 | 8p22 |
| | | | AA) | | | |
| | | | Solute carrier family 7, member 4/SLC7A4 (cationic | 603752 | ****** | 22q11.2 |
| | | | AA) | | | |
| | | | Solute carrier family 7, member 5/SLC7A5 (neutral | 600182 | M80244 | 16q24.3 |
| | | | AA) | | | |
| | | | Solute carrier family 7, member 7/SLC7A7 (dibasic | 603593 | Y18474 | 14q11.2 |
| | | | AA) | | | |
| | | | Solute carrier family 7, member 9/SLC7A9 (neutral | 604144 | ****** | 19q13.1 |
| | | | AA) | | | |
| | | | Solute carrier family 10, member 1/SLC10A1 | 182396 | NM_003049 | chr. 14 |
| | | | (taurocholate) | | | |
| | | | Solute carrier family 10, member 2/SLC10A2 | 601295 | NM_000452 | 13q33 |
| | | | (taurocholate) | | | |
| | | | Solute carrier family 11, member 1/SLC11A1 (?) | 600266 | AH002806 | 2q35 |
| | | | Solute carrier family 11, member 2/SLC11A2 (iron) | 600523 | L37347 | 12q13 |
| | | | Solute carrier family 13, member 2/SLC13A2 | 604148 | NM_003984 | 17p11.1- |
| | | | (dicarboxylic acids) | | | q11.1 |
| | | | Solute carrier family 14, member 1/SLC14A1 (urea) | 111000 | ****** | 18q11- |
| | | | | | | q12 |
| | | | Solute carrier family 14, member 2/SLC14A2 (urea) | 601611 | X96969 | 18q12.1- |
| | | | | | | q21.1 |
| | | | Solute carrier family 15, member 1/SLC15A1 | 600544 | U13173 | 13q33- |
| | | | (peptides) | | | q34 |
| | | | Solute carrier family 15, member 2/SLC15A2 | 602339 | S78203 | ****** |
| | | | (peptides) | | | |
| | | | Solute carrier family 16, member 1/SLC16A1 | 600682 | NM_003051 | 1p13.2- |
| | | | (monocarboxylic acids) | | | p12 |
| | | | Solute carrier family 16, member 2/SLC16A2 | 300095 | NM_006517 | Xq13.2 |
| | | | (monocarboxylic acids) | | | |
| | | | Solute carrier family 16, member 3/SLC16A3 | 603877 | NM_004207 | ****** |
| | | | (monocarboxylic acids) | | | |
| | | | Solute carrier family 16, member 4/SLC16A4 | 603878 | ****** | ****** |
| | | | (monocarboxylic acids) | | | |
| | | | Solute carrier family 16, member 5/SLC16A5 | 603879 | NM_004695 | ****** |
| | | | (monocarboxylic acids) | | | |
| | | | Solute carrier family 16, member 6/SLC16A6 | 603880 | NM_004694 | ****** |
| | | | (monocarboxylic acids) | | | |
| | | | Solute carrier family 16, member 7/SLC16A7 | 603654 | AF049608 | 12q13 |
| | | | (monocarboxylic acids) | | | |
| | | | Solute carrier family 18, member 1/VAT1/SLC18A1 | 193001 | L09118 | 10q25 |
| | | | (monoamines) | | | |
| | | | Solute carrier family 18, member 2/VAT2/SLC18A2 | 193002 | ****** | 8p21.3 |
| | | | (monoamines) | | | |
| | | | Solute carrier family 18, member 3/VAT3/SLC18A3 | 600336 | NM_003055 | 10q11.2 |
| | | | (monoamines) | | | |
| | | | Solute carrier family 19, member 1/SLC19A1 | 600424 | U19720 | 21q22.3 |
| | | | (reduced folate) | | | |
| | | | Solute carrier family 19, member 2/SLC19A2 | 603941 | AF160186 | 1q23.2- |
| | | | (thiamine) | | | q23.3 |
| | | | Solute carrier family 21, member 2/SLC21A2 | 601460 | NM_005630 | 3q21 |
| | | | (prostaglandin) | | | |
| | | | Solute carrier family 21, member 3/SLC21A3 | 602883 | NM_005075 | 12p12 |
| | | | (organic anion) | | | |
| | | | Solute carrier family 22, member 1/SLC22A1 | 602607 | NM_003058 | 6q26 |
| | | | (organic cation) | | | |
| | | | Solute carrier family 22, member 1-like/SLC22A1L | 602631 | AF037064 | 11p15.5 |
| | | | (organic cation) | | | |
| | | | Solute carrier family 22, member 2/SLC22A2 | 602608 | NM_003058 | 6q26 |
| | | | (organic cation) | | | |
| | | | Solute carrier family 22, member 4/SLC22A4 | 604190 | NM_003059 | Chr. 5 |
| | | | (organic cation) | | | |
| | | | Solute carrier family 22, member 5/SLC22A5 | 603377 | NM_003060 | 5q33.1 |
| | | | (camitine) | | | |
| | | | Solute carrier family 25, member 1/SLC25A1 | 190315 | X96924 | 22q11 |
| | | | (tricarboxylic acids) (mitochondrial) | | | |
| | | | Solute carrier family 25, member 11/SLC25A11 | 604165 | NM_003562 | 17p13.3 |
| | | | (oxoglutarate/malate) (mitochondrial) | | | |
| | | | Solute carrier family 25, member 12/SLC25A12 (?) | 603667 | NM_003705 | ****** |
| | | | (mitochondrial) | | | |
| | | | Solute carrier family 25, member 13/SLC25A13 (?) | 603859 | ****** | 7q21.3 |
| | | | (mitochondrial) | | | |
| | | | Solute carrier family 25, member 15/SLC25A15 | 603861 | ****** | 13q14 |
| | | | (ornithine) (mitochondrial) | | | |
| | | | Solute carrier family 25, member 16/SLC25A16 | 139080 | M31659 | 10q21.3- |
| | | | (ADP/ATP) (mitochondrial) | | | q22.1 |
| | | | Solute carrier family 29, member 1/SLC29A1/ENT1 | 602193 | NM_004955 | 6p21.2- |
| | | | (nucleoside) (mitochondrial) | | | p21.1 |
| | | | Solute carrier family 29, member 2/SLC29A2/ENT2 | 602110 | X86681 | 11q13 |
| | | | (nucleoside) (mitochondrial) | | | |
| Phase I Drug | Monooxigenases | Flavin- | Flavin-containing monooxygenase 1/FMO1 | 136130 | NM_002021 | 1q23-q25 |
| Metabolism | (mixed function | containing | Flavin-containing monooxygenase 3/FMO3 | 136132 | AH006707 | 1q23-q25 |
| (oxidation and | oxidases) | Mono- | Flavin-containing monooxygenase 4/FMO4 | 136131 | NM_001460 | 1q23-q25 |
| reduction) | | oxygenases | Flavin-containing monooxygenase 5/FMO5 | 603957 | NM_001461 | 1q21.1 |
| | | P450 | Aryl hydrocarbon receptor nuclear | 126110 | NM_001668 | 1q21 |
| | | Cytochromes | translocator/ARNT | | | |
| | | | Aryl hydrocarbon receptor nuclear translocator- | 602550 | NM_001178 | 11p15 |
| | | | like/ARNTL | | | |
| | | | Aryl hydrocarbon receptor/AHR | 600253 | NM_001621 | 7p15 |
| | | | Nuclear receptor subfamily 1, group I, member | 603065 | NM_003889 | ****** |
| | | | 2/NR1I2 | | | |
| | | | Constitutive androstane receptor, beta/orphan nuclear | 603881 | NM_005122 | ****** |
| | | | hormone receptor/CAR | | | |
| | | | Nuclear receptor subfamily 1, group H, member | 600380 | U07132 | 19q13.3 |
| | | | 2/NR1H2 | | | |
| | | | Retinoic acid receptor, alpha/RARA | 180240 | NM_000964 | 17q12 |
| | | | Retinoic acid receptor, beta/RARB | 180220 | NM_000965 | 3p24 |
| | | | Retinoic acid receptor, gamma/RARG | 180190 | M57707 | 12q13 |
| | | | Retinoid X receptor alpha/RXRA | 180245 | NM_005693 | 9q34.3 |
| | | | Retinoid X receptor beta/RXRB | 180246 | X66424 | 6p21.3 |
| | | | Retinoid X receptor gamma/RXRG | 180247 | U38480 | 1q22-q23 |
| | | | RAR-related orphan receptor A/RORA | 600825 | NM_002943 | 15q21- |
| | | | | | | q22 |
| | | | RAR-related orphan receptor B/RORB | 600825 | ****** | 15q21- |
| | | | | | | q22 |
| | | | RAR-related orphan receptor C/RORC | 602943 | NM_005060 | 1q21 |
| | | | cellular retinoic acid-binding protein, type | 180231 | ****** | 1q21.3 |
| | | | 2/CRABP2 | | | |
| | | | glucocorticoid receptor/GRL | 138040 | NM_000176 | 5q31 |
| | | | Peroxisome proliferative activated receptor, | 170998 | NM_005036 | 22q12- |
| | | | alpha/PPARA | | | q13.1 |
| | | | Peroxisome proliferative activated receptor, | 601487 | NM_005037 | 3p25 |
| | | | gamma/PPARG | | | |
| | | | Peroxisome proliferative activated receptor, | 600409 | NM_006238 | 1q21.3 |
| | | | delta/PPARD | | | |
| | | | cytochrome P450, subfamily I, polypeptide 1 (aryl | 108330 | NM_000499 | 15q22- |
| | | | hydrocarbon oxidase)/CYP1A1 | | | q24 |
| | | | cytochrome P450, subfamily I, polypeptide 2 | 124060 | AH002667 | 15q22- |
| | | | (phenacetin metabolism)/CYP1A2 | | | qter |
| | | | cytochrome P450, subfamily IB, polypeptide 1 | 601771 | NM_000104 | 2p22-p21 |
| | | | (dioxin inducible)/CYP1B1 | | | |
| | | | cytochrome P450, subfamily II, polypeptide 1 | 123960 | X13897 | 19q13.2 |
| | | | (phenobarbital inducible)/CYP2A | | | |
| | | | cytochrome P450, subfamily IIA, polypeptide 6 | 122720 | NM_000762 | 19q13.2 |
| | | | (coumarin-7-hydroxylase)/CYP2A6 | | | |
| | | | cytochrome P450, subfamily IIB (phenobarbital | 123930 | M29874 | 19q13.2 |
| | | | inducible)/CYP2B | | | |
| | | | cytochrome P450, subfamily IIC, polypeptide | 601129 | ****** | 10q24 |
| | | | 8/CYP2C8 | | | |
| | | | cytochrome P450, subfamily IIC, polypeptide 9 | 601130 | ****** | 10q24 |
| | | | (hydroxylation of tolbutamide)/CYP2C9 | | | |
| | | | Cytochromescytochrome P450, subfamily IIC, polypeptide | 601131 | ****** | 10q25 |
| | | | 18/CYP2C18 | | | |
| | | | cytochrome P450, subfamily IIC, polypeptide 19 | 124020 | NM_000769 | 10q24.1- |
| | | | (mephenytoin 4-hydroxylase)/CYP2C19 | | | q24.3 |
| | | | cytochrome P450, subfamily IID, polypeptide 6 | 124030 | NM_000106 | 22q13.1 |
| | | | (debrisoquine hydroxylation)/CYP2D6 | | | |
| | | | cytochrome P450, subfamily IIE (ethanol | 124040 | J02843 | 10q24.3- |
| | | | inducible)/CYP2E | | | qter |
| | | | cytochrome P450, subfamily IIF (ethoxycoumarin | 124070 | NM_000774 | 19q13.2 |
| | | | monooxygenase), polypeptide 1/CYP2F1 | | | |
| | | | cytochrome P450, subfamily IIJ (arachidonate | 601258 | NM_000775 | 1p31.3- |
| | | | epoxygenase), polypeptide 2/CYP2J2 | | | p31.2 |
| | | | cytochrome P450, subfamily IIIA (niphedipine | 124010 | NM_000776 | 7q22.1 |
| | | | oxidase), polypeptide 3/CYP3A3 | | | |
| | | | cytochrome P450, subfamily IVA (fatty acid W- | 601310 | NM_000778 | Chr.1 |
| | | | hydroxylase), polypeptide 11/CYP4A11 | | | |
| | | | cytochrome P450, subfamily IVB, polypeptide | 124075 | NM_000779 | 1p34-p12 |
| | | | 1/CYP4B1 | | | |
| | | | cytochrome P450, subfamily IVF (leukotriene B4-W- | 601270 | NM_000896 | 19p13.2 |
| | | | hydroxylase), polypeptide 3/CYP4F3 | | | |
| | | | cytochrome P450, subfamily VIIA (cholesterol 7-a- | 118455 | M89803 | 8q11-q12 |
| | | | hydroxylase), polypeptide 1/CYP7A1 | | | |
| | | | cytochrome P450, subfamily VIIB (oxysterol 7-a- | 603711 | AF029403 | 8q21.3 |
| | | | hydroxylase), polypeptide 1/CYP7B1 | | | |
| | | | cytochrome P450, subfamily VIIIB (sterol 12-a- | 602172 | ****** | 3p21.3- |
| | | | hydroxylase), polypeptide 1/CYP8B1 | | | p22 |
| | | | cytochrome P450, subfamily XIA (cholesterol side- | 118485 | NM_000781 | 15q23- |
| | | | chain cleavage)/CYP11A | | | q24 |
| | | | cytochrome P450, subfamily XIB, polypeptide 2 | 124080 | NM_000498 | 8q21 |
| | | | (steroid 11-b-hydroxylase)/CYP11B2 | | | |
| | | | cytochrome P450, subfamily XIX (androgen | 107910 | NM_000103 | 15q21.1 |
| | | | aromatase)/CYP19 | | | |
| | | | cytochrome P450, subfamily XXI (sterol 21-a- | 201910 | M13936 | 6p21.3 |
| | | | hydroxylase)/CYP21 | | | |
| | | | cytochrome P450, subfamily XXIV (25- | 600125 | S67623 | 20q13.2- |
| | | | hydroxyvitamin D24-hydroxylase)/CYP24 | | | q13.3 |
| | | | cytochrome P450, subfamily XXVIA, polypeptide 1 | 602239 | NM_000783 | 10q23- |
| | | | (retinoic acid hydroxylase)/CYP26A1 | | | q24 |
| | | | cytochrome P450, subfamily XXVIIA, polypeptide 1 | 213700 | NM_000105 | 2q33-qter |
| | | | (25-hydroxyvitamin D-1-a-hydroxylase)/CYP27A1 | | | |
| | | | adrenodoxin/ferredoxin 1/FDX1/ADX | 103260 | NM_004109 | 11q22 |
| | | | adrenodoxin reductase/ferredoxin:NADP(+) | 103270 | NM_004110 | 17q24- |
| | | | reductase/FDXR/ADXR | | | q25 |
| | | | cytochrome P450, subfamily XXVIIB, polypeptide 1 | 264700 | NM_000785 | 12q14 |
| | | | (25-hydroxyvitamin D-1-a-hydroxylase)/CYP27B1 | | | |
| | | | cytochrome P450, subfamily XLVI (cholesterol 24- | 604087 | NM_006668 | 14q32.1 |
| | | | hydroxylase)/CYP46 | | | |
| | | | cytochrome P450, subfamily LI (lanosterol 14-a- | 601637 | U51692 | 7q21.2- |
| | | | demethylase)/CYP51 | | | q21.3 |
| | General | General | Monoamine Oxidase A; MAOA | 309850 | M69226 | Xp11.23 |
| | Oxidases | Oxidases | Monoamine Oxidase B; MAOB | 309860 | M69177 | Xp11.23 |
| | | | Copper-containing amine oxidase/AOC3 | 603735 | NM_003734 | 17q21 |
| | | | Xanthine dehydrogenase/XDH | 278300 | NM_000379 | 2p23-p22 |
| | | | tryptophan 2,3-dioxygenase/TDO2 | 191070 | NM_005651 | 4q31-q32 |
| | | | sulfite oxidase/SUOX | 272300 | NM_000456 | ****** |
| | Dehydrogenases | Cofactor | molybdenum factor synthesis 1/MOCS1 | 603707 | AJ224328 | 6p21.3 |
| | | Synthesis | molybdenum factor synthesis 2/MOCS2 | 603708 | ****** | 5q11 |
| | | Alcohol | alcohol dehydrogenases 1, alpha subunit/ADH1 | 103700 | NM_000667 | 4q22 |
| | | Dehydrogenases | alcohol dehydrogenases 2, beta subunit/ADH2 | 103720 | NM_000668 | 4q22 |
| | | | alcohol dehydrogenases 3, gamma subunit/ADH3 | 103730 | M12272 | 4q22 |
| | | | alcohol dehydrogenases 4/pi isozyme/ADH4 | 103740 | M15943 | 4q22 |
| | | | alcohol dehydrogenases 5/chi isozyme/ADH5 | 103710 | NM_000671 | 4q21-q25 |
| | | | alcohol dehydrogenases 6/ADH6 | 103735 | NM_000672 | 15q26 |
| | | | alcohol dehydrogenases 7/ADH7 | 600086 | AH006682 | 4q23-q24 |
| | | Aldehyde | aldehyde dehydrogenase 1/ALDH1 (liver cytosol) | 100640 | AH002598 | 9q21 |
| | | Dehydrogenases | aldehyde dehydrogenase 2/ALDH2 (liver | 100650 | K03001 | 12q24.2 |
| | | | mitochondria) | | | |
| | | | aldehyde dehydrogenase 3/acetaldehyde | 100660 | M74542 | 17p11.2 |
| | | | dehydrogenase/ALDH3 (stomach) | | | |
| | | | aldehyde dehydrogenase 5/acetaldehyde | 100670 | NM_000692 | 9p13 |
| | | | dehydrogenase/ALDH5 | | | |
| | | | aldehyde dehydrogenase 5, member Al/succinic | 271980 | NM_001080 | 6p22 |
| | | | semialdehyde dehydrogenase/ALDH5A1 | | | |
| | | | aldehyde dehydrogenase 6/acetaldehyde | 600463 | NM_000693 | 15q26 |
| | | | dehydrogenase/ALDH6 | | | |
| | | | aldehyde dehydrogenase 7/acetaldehyde | 600466 | NM_000694 | 11q13 |
| | | | dehydrogenase/ALDH7 | | | |
| | | | aldehyde dehydrogenase 8/ALDH8 | 601917 | NM_000695 | chr. 11 |
| | | | aldehyde dehydrogenase 9/g-aminobutyraldehyde | 602733 | NM_000696 | 1q22-q23 |
| | | | dehydrogenase/ALDH9 | | | |
| | | | aldehydedehydrogenase 10/ALDH10 | 270200 | NM_000382 | 17p11.2 |
| | | Dihydro- | Dihydropyrimidine dehydrogenase (5-fluoroacil | 274270 | U09178 | 1p22 |
| | | pyrimidine | detoxification) | | | |
| | | Dehydrogenase | | | | |
| | Fatty Acid β- | Peroxisome | Peroxisome proliferative activated receptor, | 170998 | NM_005036 | 22q12- |
| | Oxidation | Proliferation | alpha/PPARA | | | q13.1 |
| | | | Peroxisome proliferative activated receptor, | 601487 | NM_005037 | 3p25 |
| | | | gamma/PPARG | | | |
| | | | Peroxisome proliferative activated receptor, | 180231 | NM_006238 | 1q21.3 |
| | | | delta/PPARD | | | |
| | | Peroxisome | peroxisome biogenesis factor 1/PEX1 | 602136 | AB008112 | 7g21-q22 |
| | | Synthesis | peroxisomal membrane protein 3 (35kD, Zeliweger | 170993 | NM_000318 | 8q21.1 |
| | | | syndrome)/PXMP3/PEX2 | | | |
| | | | peroxisomal biogenesis factor 3/PEX3 | 603164 | NM_003630 | ****** |
| | | | peroxisomal biogenesis factor 6/PEX6 | 601498 | NM_000287 | 6p21.1 |
| | | | peroxisomal biogenesis factor 7/PEX7 | 601757 | NM_000288 | 6q22-q24 |
| | | | peroxisomal biogenesis factor 10/PEX10 | 602859 | ****** | ****** |
| | | | peroxisomal biogenesis factor 11A/PEX11A | 603866 | NM_003847 | ****** |
| | | | peroxisomal biogenesis factor 11B/PEX11B | 603867 | NM_003846 | ****** |
| | | | peroxisomal biogenesis factor 12/PEX12 | 601758 | NM_000286 | |
| | | | peroxisomal biogenesis factor 13/PEX13 | 601789 | U71374 | 2p15 |
| | | | peroxisomal biogenesis factor 14/PEX14 | 601791 | ****** | ****** |
| | | | peroxisomal farnesylated protein/PXF/peroxisomal | 600279 | NM_002857 | 1q22 |
| | | | biogenesis factor 19/PEX19 | | | |
| | | Coenzyme A | Fatty acid CoA Ligase, long chain 1/FACL1 | 152425 | ****** | 3q13 |
| | | Ligases | Fatty acid CoA Ligase, long chain 2/FACL2 | 152426 | ****** | 4q34-q35 |
| | | | Fatty acid CoA Ligase, long chain 3/FACL3 | 602371 | NM_004457 | 2q34-q35 |
| | | | Fatty acid CoA Ligase, long chain 4/FACL4 | 300157 | NM_004458 | Xq22.3 |
| | | | Fatty acid CoA Ligase, very long chain 1/FACVL1 | 603247 | ****** | 15q21.2 |
| | | Oxidation | Enoyl-CoA, hydratase/3-hydroxyacyl CoA | 261515 | NM_001966 | 3q27 |
| | | | dehydrogenase/EHHADH | | | |
| | | | hydroxyacyl-CoA dehydrogenase/3-ketoacyl-CoA | 600890 | NM_000182 | 2p23 |
| | | | thiolase/enoyl-CoA hydratase, alpha | | | |
| | | | subunit/HADHA | | | |
| | | | hydroxyacyl-CoA dehydrogenase/3-ketoacyl-CoA | 143450 | NM_000183 | 2p23 |
| | | | thiolase/enoyl-CoA hydratase, beta subunit/HADHB | | | |
| | | | acyl-Coenzyme A oxidase 1/ACOX1 (peroxisomal) | 264470 | NM_004035 | 17q25 |
| | | | acyl-Coenzyme A oxidase 2, branched chain/ACOX2 | 601641 | NM_003500 | 13pl4.13 |
| | | | (peroxisomal) | | | |
| | | | acyl-Coenzyme A dehydrogenase, C-2 to C-3 short | 201470 | NM_000017 | 12q22- |
| | | | chain precursor/ACADS (mitochondrial) | | | qter |
| | | | acyl-Coenzyme A dehydrogenase, C-4 to C-12 | 201450 | NM_000016 | 1p31 |
| | | | straight chain/ACADM (mitochondrial) | | | |
| | | | acyl-Coenzyme A dehydrogenase, long | 201460 | NM_001608 | 2q34-q35 |
| | | | chain/ACADL (mitochondrial) | | | |
| | | | hydroxyacyl-Coenzyme A dehydrogenase, type | 602057 | NM_004493 | ****** |
| | | | II/HADH2 | | | |
| | | | enoyl-Coenzyme A hydratase 1/ECH1 (peroxisomal) | 600696 | NM_001398 | 19q13 |
| | Reduction | Aldo-Keto | Aldo-keto reductase family 1, member | 103830 | NM_006066 | 1p33-p32 |
| | | Reductases | A1/dihydrodiol dehydrogenase/AKR1A1 | | | |
| | | | Aldo-keto reductase family 1, member | 600449 | NM_001353 | 10p15- |
| | | | C1/dihydrodiol dehydrogenase/AKR1C1 | | | p14 |
| | | | Aldo-keto reductase family 1, member | 603966 | NM_003739 | 10p15- |
| | | | C3/dihydrodiol dehydrogenase/AKR1C3 | | | p14 |
| | | | Aldo-keto reductase family 1, member | 600451 | ****** | 10p15- |
| | | | C4/chlorodecone reductase/AKR1C4 | | | p14 |
| | | | Aldo-keto reductase family 7, member A2/aflatoxin | 603418 | NM_003689 | ****** |
| | | | aldehyde reductase/AKR7A2 | | | |
| | | | Carbonyl reductase 1/CBR1 | 114830 | NM_001757 | 21q22.12 |
| | | | Carbonyl reductase 2/CBR2 | ****** | ****** | chr. 11 |
| | | | Carbonyl reductase 3/CBR3 | 603608 | NM_001236 | 21q22.2 |
| | | | Sepiapterin reductase (7,8-dihydrobiopterin:NADP+ | 182125 | NM_003124 | 2p14-p12 |
| | | | oxidoreductase)/SPR | | | |
| | | Quinone | Z-crystallin/quinone reductase/CRYZ | 123691 | L315211 | p31-p22 |
| | | Oxidoreductases | Z-crystallin-like/quinone reductase-like/CRYZL1 | 603920 | NM_005111 | 21q22.1 |
| | | | NAD(P)H menadione oxidoreductase 1, dioxin- | 125860 | NM_000903 | 16q22.1 |
| | | | inducible/NMOR1/diaphorase 4/DIA4 | | | |
| | | | NAD(P)H menadione oxidoreductase 2, dioxin- | 160998 | NM_000904 | 6pter-q12 |
| | | | inducible/NMOR2 | | | |
| | Conjugation | Sulfate Unit | 3-prime-phosphoadenosine 5-prime-phosphosulfate | 603262 | NM_005443 | 4q |
| | | Activation | synthase 1/PAPSS1 | | | |
| | | | Phenol-preferring sulfotransferase, family 1A, | 171150 | NM_001055 | 16p12.1- |
| | | | member 1/SULT1A1 | | | p11.2 |
| | | | Phenol-preferring sulfotransferase, family 1A, | 601292 | NM_001054 | 16p12.1- |
| | | | member 2/SULT1A2 | | | p11.2 |
| | | | Phenol-preferring sulfotransferase, family 1A, | 600641 | L19956 | 16p11.2 |
| | | | member 3/SULT1A3 | | | |
| | | | Sulfotransferase, family 1C, member 3/SULT1C1 | 602385 | U66036 | 2q11.1- |
| | | | | | | q11.2 |
| | | | Dehydroepiandrosterone (DHEA)-preferring | 125263 | NM_003167 | 19q13.3 |
| | | | sulfotransferase, family 2A, member 1/SULT2A1 | | | |
| | | | Sulfotransferase, family 2B, member 1/SULT2B1 | 604125 | NM_004605 | 19q13.3 |
| | | | Estrogen-preferring sulfotransferase/STE | 600043 | NM_005420 | 4q13.1 |
| | | | N-deacetylase/N-sulfotransferase (heparan | 600853 | U18918 | 5q32- |
| | | | glucosaminyl)/NDST1 | | | q33.3 |
| | | | N-deacetylase/N-sulfotransferase (heparan | 603268 | NM_003635 | 10q22 |
| | | | glucosaminyl)/NDST2 | | | |
| | | | N-deacetylase/N-sulfotransferase (heparan | 603950 | NM_004784 | ****** |
| | | | glucosaminyl)/NDST3 | | | |
| | | | Carbohydrate sulfotransferase 1 (chondroitin | 603797 | NM_003654 | 11p11.2- |
| | | | 6/keratan)/CHST1 | | | p11.1 |
| | | | Carbohydrate sulfotransferase 2 (chondroitin | 603798 | ****** | 7q31 |
| | | | 6/keratan)/CHST2 | | | |
| | | | Carbohydrate sulfotransferase 3 (chondroitin | 603799 | NM_004273 | ****** |
| | | | 6/keratan)/CHST3 | | | |
| | | | Cerebroside sulfotransferase (3′- | 602300 | NM_004861 | ****** |
| | | | phosphoadenylylsulfate:galactosylceramide 3′)/CST | | | |
| | | | Heparan sulfate (glucosamine) 3-O-sulfotransferase | 603244 | NM_005114 | ****** |
| | | | 1/HS3ST1 | | | |
| | | | Heparan sulfate (glucosamine) 3-O-sulfotransferase | 604056 | NM_006043 | 16p12 |
| | | | 2/HS3ST2 | | | |
| | | | Heparan sulfate (glucosamine) 3-O-sulfotransferase | 604057 | NM_006042 | 17p12- |
| | | | 3A1/HS3ST3A1 | | | p11.2 |
| | | | Heparan sulfate (glucosamine) 3-O-sulfotransferase | 604058 | NM_006041 | 17p12- |
| | | | 3B1/HS3ST3B1 | | | p11.2 |
| | | | Heparan sulfate (glucosamine) 3-O-sulfotransferase | 604059 | AF105378 | 16p11.2 |
| | | | 4/HS3ST4 | | | |
| | | Sulfhydrylation | Methylguanine methyltransferase (O6-alkylguanine | 156569 | M29971 | 10q26 |
| | | | detoxification) | | | |
| | | | thiosulfate thiotransferase/rhodanese/TST (cyanide | 180370 | D87292 | 22q11.2- |
| | | | detoxification) | | | qter |
| | | UDP- | UDP glycosyltransferase 1/UGT1 | 191740 | NM_001072 | Chr. 12 |
| | | Glycosyltransfer | UDP glycosyltransferase family 2, member | 600067 | NM_001073 | 4q13 |
| | | ases | B4/UGT2B4 | | | |
| | | | UDP glycosyltransferase family 2, member | 600068 | NM_001074 | 1q14 |
| | | | B7/UGT2B7 | | | |
| | | | UDP glycosyltransferase family 2, member | 600070 | NM_001075 | ****** |
| | | | B10/UGT2B10 | | | |
| | | | UDP glycosyltransferase family 2, member | 600069 | U06641 | 4q13 |
| | | | B15/UGT2B15 | | | |
| | | | UDP glycosyltransferase family 2, member | 601903 | NM_001077 | 1q14 |
| | | | B17/UGT2B17 | | | |
| | | | UDP glycosyltransferase 8/UGT8 | 601291 | U30930 | 4q26 |
| | | | UDP-glucuronosyltransferase | 218800 | AJ005162 | Chr.2 |
| | | Carbon Unit | methionine adenosyltransferase I, alpha/MAT1A | 250850 | NM_000429 | 10q22 |
| | | Activation for | methionine adenosyltransferase II, alpha/MAT2A | 601468 | NM_005911 | 2p11.2 |
| | | SAM | | | | |
| | | Carbon Unit | Folate Receptor Alpha/FOLR1 | 136430 | M28099 | 11q13.3- |
| | | Activation for | | | | g13.5 |
| | | Folate | Folate Receptor Beta/FOLR2 | 136425 | AF000380 | 11q13.3- |
| | | | | | | q13.5 |
| | | | Folate Receptor Gamma/FOLR3 | 602469 | Z32564 | ****** |
| | | | Folate Transporter (SLC19A1) | 600424 | U19720 | 21q22.3 |
| | | | Vitamin B12 binding protein | 275350 | NM_000355 | 22q11.2- |
| | | | | | | qter |
| | | | folylpolyglutamate synthetase/FPGS | 136510 | M98045 | 9cen-q34 |
| | | | gamma-glutamyl hydrolase/GGH | 601509 | U55206 | ****** |
| | | | Methylenetetrahydrofolate reductase/MTHFR | 236250 | U09806 | 1p36.3 |
| | | | Dihydrofolate reductase/DHFR | 126060 | J00140 | 5q11.2- |
| | | | | | | q13.2 |
| | | | 5,10-methylenetetrahydrofolate dehydrogenase, 5,10- | 172460 | NM_005956 | 14q24 |
| | | | methylenetetrahydrofolate cyclohydrolase, 10- | | | |
| | | | formyltetrahydrofolate synthetase/MTHFD1 | | | |
| | | | 5,10-methenyltetrahydrofolate synthetase (5- | 604197 | NM_006441 | Chr. 15 |
| | | | formyltetrahydrofolate cyclo-ligase)/MTHFS | | | |
| | | | phosphoribosylglycinamide formyltransferase, | 138440 | NM_000819 | 21q22.1 |
| | | | phosphoribosylglycinamide synthetase, | | | |
| | | | phosphoribosylaminoimidazole synthetase/GART | | | |
| | | | folate hydrolase 1/FOH1 | 600934 | NP004467 | 11q14 |
| | | | 6-pyruvoyl tetrahydrobiopterin synthase/PTPS | 261640 | Q03393 | 11q22.3- |
| | | | | | | q23.3 |
| | | | serine hydroxymethyltransferase 1 (soluble)/SHMT1 | 182144 | NM_004169 | 17p11.2 |
| | | | senile hydroxymethyltransferase 2 | 138450 | NM_005412 | 12q13 |
| | | | (mitochondrial)/SHMT2 | | | |
| | | | Glycine aminotransferase/glycine cleavage T | 238310 | NM_000481 | 3p21.2- |
| | | | protein/GAT | | | p21.1 |
| | | | 5-methyltetrahydrofolate-homocysteine | 156570 | NM_000254 | 1q43 |
| | | | methyltransferase/methionine synthase/MTR | | | |
| | | | glutamate formiminotransferase/dihydrofolate | 229100 | ****** | ****** |
| | | | synthetase | | | |
| | | Methylation | catechol-O-methyltransferase/COMT | 116790 | NM_000754 | 22q11.2 |
| | | | phenylethanolamine N-methyltransferase/PNMT | 171190 | NM_002686 | 17q21- |
| | | | | | | q22 |
| | | | nicotinamide N-methyltransferase/NNMT | 600008 | NM_006169 | 11q23.1 |
| | | | Thiopurine methyltransferase (6-mercaptopurine | 187680 | U12387 | 6p22.3 |
| | | | detoxification) | | | |
| | | Carbon Unit | pyruvate dehydrogenase E1-alpha subunit/PDHA1 | 312170 | L48690 | Xp22.2- |
| | | Activation for | | | | p22.1 |
| | | Acetyl-CoA | pyruvate dehydrogenase (lipoamide) beta/PDHB | 179060 | NM_000925 | 3p13-q23 |
| | | | Acetyl-CoA pyruvate dehydrogenase complex, lipoyl- | 245349 | NM_003477 | 11p13 |
| | | | containing component X/E3-binding protein/PDX1 | | | |
| | | | pyruvate dehydrogenase complex E3 subunit/DLD | 246900 | NM_000108 | 7q31-q32 |
| | | Acylation | sterol-O-acyl transferase 1/SOAT1 | 102642 | L21934 | 1q25 |
| | | | sterol-O-acyl transferase 2/SOAT2 | 601311 | ****** | chr. 12 |
| | | | N-acetyltransferase 1/arylamide acetylase 1/NAT1 | 108345 | NM_000662 | 8p23.1- |
| | | | | | | p21.3 |
| | | | N-acetyltransferase 2/arylamide acetylase 2/NAT2 | 243400 | NM_000015 | 8p23.1- |
| | | | | | | p21.3 |
| | | Glutathione | Glutathione-S-transferase 6 | 138391 | ****** | ****** |
| | | Transferase | | | | |
| | | | Glutathione-S-transferase, alpha 1/GSTA1 | 138359 | L13269 | 6p12.2 |
| | | | Glutathione-S-transferase, alpha 2/GSTA2 | 138360 | M15872 | 6p12.2 |
| | | | Glutathione-S-transferase, kappa 1/GSTK1 | 602321 | ****** | ****** |
| | | | Glutathione-S-transferase 1/MGST1 (microsomal) | 138330 | AH003674 | Chr. 12 |
| | | | Glutathione-S-transferase 2/MGST2 (microsomal) | 601733 | NM_002413 | 4q28-q31 |
| | | | Glutathione-S-transferase, mu 1-like/GSTM1L | 138270 | ****** | Chr. 3 |
| | | | Glutathione-S-transferase, mu 1/GSTM1 | 138350 | J03817 | 1p13.3 |
| | | | Glutathione-S-transferase, mu 2/GSTM2 (muscle) | 138380 | NM_000848 | 1p13.3 |
| | | | Glutathione-S-transferase, mu 3/GSTM3 (brain) | 138390 | NM_000849 | 1p13.3 |
| | | | Glutathione-S-transferase, mu 4/GSTM4 | 138333 | NM_000850 | 1p13.3 |
| | | | Glutathione-S-transferase, mu 5/GSTM5 (brain/lung) | 138385 | NM_000851 | 1p13.3 |
| | | | Glutathione-S-transferase, pi/GSTP1 | 134660 | NM_000852 | 11q13 |
| | | | Glutathione-S-transferase, theta 1/GSTT1 | 600436 | NM_000853 | 22q11.2 |
| | | | Glutathione-S-transferase, theta 2/GSTT2 | 600437 | NM_000854 | 22q11.3 |
| | | | Glutathione-S-transferase, zeta 1 /maleylacctoactetate | 603758 | NM_001513 | 14q24.3 |
| | | | isomerase/MAAI/GSTZ1 | | | |
| | | γ-Glutamyl- | Gamma-glutamyltranspeptidase 1/GGT1 | 231950 | J04131 | 22q11.1- |
| | | transpeptidase | | | | q11.2 |
| | | | Gamma-glutamyltranspeptidase 2/GGT2 | 137181 | AH002728 | 22q11.1 |
| | | | Gamma-glutamyltransferase-like activity 1/GGTLA1 | 137168 | NM_004121 | ****** |
| | Catabolism | Esterases | paraoxonase 1/PON1 (arylesterase) | 168820 | AH004193 | 7q21.3 |
| | | | paraoxonase 2/PON2 | 602447 | L48513 | 7q21.3 |
| | | | paraoxonase 3/PON3 | 602720 | ****** | 7q21.4 |
| | | | esterase C/ESC (acetyl esterase) | 133270 | | |
| | | | esterase A4/ESA4 | 133220 | ****** | 11q13- |
| | | | | | | q22 |
| | | | esterase B/buteryl esterase/ESB (erythrocyte) | 133260 | ****** | ****** |
| | | | esterase B3/ESB3 | 133290 | ****** | Chr. 16 |
| | | | esterase A5/A7/acetylesterase/ESA5/ESA7 (brain) | 133230 | ****** | ****** |
| | | | acetylcholinesterase/ACHE | 100740 | M55040 | 7q22 |
| | | | butyrylcholinesterase 1/serum cholinesterase | 177400 | NM_000055 | 3q26.1- |
| | | | 1/BCHE1 | | | q26.2 |
| | | | butyrylcholinestarase 2/serum cholinesterase | 177500 | ****** | 2q33-q35 |
| | | | 2/BCHE2 | | | |
| | | | carboxylesterase 1/serine esterase/CES1 (hepatic) | 114835 | SEG_HUM | 16q13- |
| | | | | | CESTG | q22.1 |
| | | | arylacetamide deacetylase/AADAC | 600338 | NM_001086 | 3q21.3- |
| | | | | | | q25.2 |
| | | Thioesterase | acyl-CoA thioester hydrolase 1, long chain/acyl-CoA | 602586 | ****** | ****** |
| | | | thioesterase 1/ACT1 | | | |
| | | | acyl-CoA thioester hydrolase 2, long chain/acyl-CoA | 602587 | ****** | ****** |
| | | | thioesterase 2/ACT2 | | | |
| | | | esterase D/S-forinylglutathion hydrolase/ESC | 133280 | M13450 | 13q14.11 |
| | | | (thioesterase) | | | |
| | | Amidase | aminoacylase 1/ACY1 | 104620 | NM_000666 | 3p21.1 |
| | | | laminoacylase 2/ACY2/aspartoacylase (Canavan | 271900 | NM_000049 | 17pter- |
| | | | disease)/ASPA | | | p13 |
| | | | fatty acid amide hydrolase/FAAH | 602935 | NM_001441 | 1p34-p35 |
| | | Epoxide | epoxide hydrolase 1/EPHX1 (microsomal) | 132810 | NM_000120 | 1p11-qter |
| | | Hydratases | epoxide hydrolase 2/EPHX2 (cytosolic) | 1132811 | ****** | 8p21-p12 |
| | | Proteases | bleomycin hydrolase/BLMH | 602403 | NM_000386 | 17q11.2 |
| Excretion | Canalicular | Transporters | Multidrug resistance protein MDR3/P-glycoprotein | 602347 | X06181 | 7q21.1 |
| | Uptake and | | 3/PGY3 | | | |
| | Concentration | | Familial intrahepatic cholestasis 1, (progressive, | 602397 | NM_005603 | 18q21 |
| | | | Byler disease and benign recurrent) /FIC1 | | | |
| | | | Bile salt export pump/BSEP | 603201 | NM_003742 | 2q24 |
| | | | Microsomal triglyceride transfer protein large | 157147 | NM_000253 | 4q22-q24 |
| | | | subunit/MTP | | | |
| | | | Solute carrier family 6, member 6/SLC6A6 (taurine) | 186854 | U16120 | 3p25-q24 |
| | | | Solute carrier family 10, member 1/SLC10A1 | 182396 | NM_003049 | chr. 14 |
| | | | (taurocholate) | | | |
| | | | Solute carrier family 10, member 2/SLC10A2 | 601295 | NM_000452 | 13q33 |
| | | | (taurocholate) | | | |
| | | | Solute carrier family 13, member 2/SLC13A2 | 604148 | NM_003984 | 17p11.1- |
| | | | (dicarboxylic acids) | | | q11.1 |
| | | | Solute carrier family 19, member, 1/SLC19A1 | 600424 | U19720 | 21q22.3 |
| | | | (reduced folate) | | | |
| | | | Solute carrier family 21, member 3/SLC21A3 | 602883 | NM_005075 | 12p12 |
| | | | (organic anion) | | | |
| | | | Solute carrier family 22, member 1/SLC22A2 | 602607 | NM_003058 | 6q26 |
| | | | (organic cation) | | | |
| | | | multidrug resistance protein MDR1 | 171050 | X96395 | 7q21.1 |
| | | | multidrug resistance associated protein | 601107 | NM_000392 | 10q24 |
| | | | MRP2/CMOAT | | | |
| | | | multidrug resistance protein MDR3/P-glycoprotein | 602347 | X06181 | 7q21.1 |
| | | | 3/PGY3 | | | |
| | | Bile Salt | Bile acid Coenzyme A: amino acid N-acyltransferase | 602938 | NM_001701 | 9q22.3 |
| | | Synthesis | (glycine N-choloyltransferase)/BAAT | | | |
| | | | cytochrome P450, subfamily XLVI (cholesterol 24- | 604087 | NM_006668 | 14q32.1 |
| | | | hydroxylase)/CYP46 | | | |
| | | | cytochrome P450, subfamily VIIA (cholesterol 7-a- | 118455 | M89803 | 8q11-q12 |
| | | | hydroxylase), polypeptide 1/CYP7A1 | | | |
| | | | cytochrome P450, subfamily VIIB (oxysterol 7-a- | 603711 | AF029403 | 8q21.3 |
| | | | hydroxylase), polypeptide 1/CYP7B1 | | | |
| | | | ATPase, Na+/K+ transporting, alpha 1 | 182310 | NM_000701 | 1p13-p11 |
| | | | polypeptide/ATP1A1 | | | |
| | | | ATPase, Na+/K+ transporting, alpha 1 polypeptide- | 182360 | NM_001676 | 13q12.1- |
| | | | like/ATP1A1L | | | q12.3 |
| | | | ATPase, Na+/K+ transporting, alpha 2 | 182340 | NM_000702 | 1q21-q23 |
| | | | polypeptide/ATP1A2 | | | |
| | | | ATPase, Na+/K+ transporting, beta 1 | 182330 | NM_001677 | 1q22-q25 |
| | | | polypeptide/ATP1B1 | | | |
| | | | ATPase, Na+/K+ transporting, beta 2 | 182331 | X16645 | 17p |
| | | | polypeptide/ATP1B2 | | | |
| | | | ATPase, Na+/K+ transporting, beta 3 | 601867 | NM_001679 | 3q22-q23 |
| | | | polypeptide/ATP1B3 | | | |
| | | | solute carrier family 4, bicarbonate/chloride anion | 109270 | NM_000342 | 17q21- |
| | | | exchanger, member 1/SLC4A1 | | | q22 |
| | | | solute carrier family 4, sodium bicarbonate | 603345 | NM_003759 | 4q21 |
| | | | cotransporter, member 4/SLC4A4 | | | |
| | | | solute carrier family 4, sodium bicarbonate | 603318 | NM_003788 | 4q21 |
| | | | cotransporter, member 5/SLC4A5 | | | |
| | | | Solute carrier family 9, member A2/SLC9A2 | 600530 | NM_003048 | 2q11.2 |
| | | | (sodium/hydrogen ion) | | | |
| | | | Solute carrier family 9, member A3/SLC9A3 | 182307 | ****** | 5p15.13 |
| | | | (sodium/hydrogen ion) | | | |
| | | | chloride channel 5/CLCN5 | 300008 | NM_000084 | Xp11.22 |
| | | | chloride channel, calcium activated, family member | 603906 | NM_001285 | 1p31-p22 |
| | | | 1/CLCA1 | | | |
| | | | chloride channel, calcium activated, family member | 604003 | NM_006536 | ****** |
| | | | 2/CLCA2 | | | |
| | | | cystic fibrosis transmembrane conductance | 602421 | NM_000492 | 7q31.2 |
| | | | regulator/CFTR | | | |
| | | | aquaporin 1/AQP1 | 107776 | NM_000385 | 7p14 |
| | | | aquaporin 3/AQP3 | 600170 | NM_004925 | 9p13 |
| | | Bile Secretion | Cholecystokinin/CCK | 118440 | L00354 | 3pter-p21 |
| | | | Cholecystokinin A receptor/CCKAR | 118444 | L13605 | 4p15.2- |
| | | | | | | p15.1 |
| | | | Cholecystokinin B receptor/CCKBR | 118445 | L08112 | 11p15.5- |
| | | | | | | p15.4 |
| | Renal Tubular | Deconjugating | Cytoplasmic cysteine conjugate-beta lyase/glutamine | 600547 | NM_004059 | Chr.9 |
| | Uptake and | Enzymes | transaminase K/CCBL1 | | | |
| | Concentration | | Galactosamine (N-acetyl)-6-sulfate sulfatase | 253000 | NM_000512 | 16q24.3 |
| | | | (Morquio syndrome)/GALNS | | | |
| | | | Iduronate-2-sulfatase (Hunter syndrome)/IDS | 309900 | NM_000202 | Xq28 |
| | | | Arylsulfatase Alsteroid sulfatase/ARSA | 250100 | NM_000487 | 22q13.31- |
| | | | | | | qter |
| | | | Arylsulfatase B/steroid sulfatase/ARSB | 253200 | NM_000046 | 5q11-q13 |
| | | | Arylsulfatase C, isozyme s/steroid sulfatase/ARCS | 308100 | NM_000351 | Xp22.32 |
| | | | Arylsulfatase D/steroid sulfatase/ARSD | 300002 | ****** | Xp22.3 |
| | | | Arylsulfatase F/steroid sulfatase/ARSE | 300180 | NM_000047 | Xp22.3 |
| | | | Arylsulfatase F/steroid sulfatase/ARSF | 300003 | NM_004042 | Xp22.3 |
| | | Uptake and | renal transport of beta-amino acids/AABT | 109660 | ****** | Chr.21 |
| | | Reuptake | Solute carrier family 3 member 1/SLC3A1 (aa | 104614 | ****** | 2p16.3 |
| | | Transporters | transporter) | | | |
| | | | Solute carrier family 5 member 2/SLC5A5 | 182381 | A56765 | 16p11.2 |
| | | | (Na/glucose transporter) | | | |
| | | | Solute carrier family 6, member 6/SLC6A6 (taurine) | 186854 | U16120 | 3p25-q24 |
| | | | Solute carrier family 7, member 9/SLC7A9 (neutral | 604144 | ****** | 19q13.1 |
| | | | AA) | | | |
| | | | Solute carrier family 13, member 2/SLC13A2 | 604148 | NM_003984 | 17p11.1- |
| | | | (dicarboxylic acids) | | | q11.1 |
| | | | solute carrier family 17 (sodium phosphate), member | 182308 | NM_005074 | 6p23- |
| | | | 1/SLC17A1 | | | p21.3 |
| | | | Solute carrier family 22, member 1/SLC22A2 | 602607 | NM_003058 | 6q26 |
| | | | (organic cation) | | | |
| | | | Solute carrier family 22, member 1-like/SLC22A1L | 602631 | AF037064 | 11p15.5 |
| | | | (organic cation) | | | |
| | | | Solute carrier family 22, member 4/SLC22A4 | 604190 | NM_003059 | Chr. 5 |
| | | | (organic cation) | | | |
| | | | Solute carrier family 22, member 5/SLC22A5 | 603377 | NM_003060 | 5q33.1 |
| | | | (carnitine) | | | |
| | | | Solute carrier family 34, member 1/SLC34A1 | 182309 | NM_003052 | 5q35 |
| | | | (sodium phosphate) | | | |
| | | Acidosis | H+-ATPase beta 1 subunit /ATP6B1 | 267300 | AH007312 | 2cen-q13 |
| | | | solute carrier family 4, sodium bicarbonate | 603345 | NM_003759 | 4q21 |
| | | | cotransporter, member 4/SLC4A4 | | | |
| | | | solute carrier family 4, sodium bicarbonate | 603318 | NM_003788 | 4q21 |
| | | | cotransporter, member 5/SLC4A5 | | | |
| | | | carbonic anhydrase II/CA2 | 259730 | NM_000067 | 8q22 |
| | | | carbonic anhydrase IV/CA4 | 114760 | NM_000717 | 17q23 |
| | | | carbonic anhydrase XII/CA12 | 603263 | AF051882 | 15q22 |
| | | | solute carrier family 4, bicarbonate/chloride anion | 109270 | NM_000342 | 17q21- |
| | | | exchanger, member 1/SLC4A1 | | | q22 |
| | | | Solute carrier family 9, member A1/SLC9A1 | 107310 | M81768 | 1p36.1- |
| | | | (sodium/hydrogen ion) | | | p35 |
| | | | Solute carrier family 9, member A2/SLC9A2 | 600530 | NM_003048 | 2q11.2 |
| | | | (sodium/hydrogen ion) | | | |
| | | | Solute carrier family 9, member A3/SLC9A3 | 182307 | ****** | 5p15.3 |
| | | | (sodium/hydrogen ion) | | | |
| | | Lithosis | Solute carrier family 13, member 2/SLC13A2 | 604148 | NM_003984 | 17p11.1- |
| | | | (dicarboxylic acids) | | | q11.1 |
| | | Sodium | 3′(2′), 5′-bisphosphate nucleotidase 1/BPNT | 604053 | NM_006085 | ****** |
| | | Tolerance | | | | |
| | | Urine | chloride channel 5/CLCN5 | 300008 | NM_000084 | Xp11.22 |
| | | Concentration | chloride channel Ka, kidney/CLCNKA | 602024 | NM_004070 | 1p36 |
| | | | chloride channel Kb, kidney/CLCNKB | 602023 | NM_000085 | 1p36 |
| | | | solute carrier family 12 (sodium/potassium/chloride | 600839 | NM_000338 | 15q15- |
| | | | transporters), member 1/SLC12A1 | | | q21.1 |
| | | | solute carrier family 12 (sodium/potassium/chloride | 600840 | NM_001046 | 5q23.3 |
| | | | transporters), member 2/SLC12A2 | | | |
| | | | solute carrier family 12 (sodium/chloride | 600968 | NM_000339 | 16q13 |
| | | | transporters), member 3/SLC12A3 | | | |
| | | | ATPase, Na+/K+ transporting, alpha 1 | 182310 | NM_000701 | 1p13-p11 |
| | | | polypeptide/ATP1A1 | | | |
| | | | ATPase, Na+/K+ transporting, alpha 1 polypeptide- | 182360 | NM_001676 | 13q12.1- |
| | | | like/ATP1A1L | | | q12.3 |
| | | | ATPase, Na+/K+ transporting, alpha | 2182340 | NM_000702 | 1q21-q23 |
| | | | polypeptide/ATP1A2 | | | |
| | | | ATPase, Na+/K+ transporting, beta 1 | 182330 | NM_001677 | 1q22-q25 |
| | | | polypeptide/ATP1B1 | | | |
| | | | ATPase, Na+/K+ transporting, beta 2 | 182331 | X16645 | 17p |
| | | | polypeptide/ATP1B2 | | | |
| | | | ATPase, Na+/K+ transporting, beta 3 | 601867 | NM_001679 | 3q22-q23 |
| | | | polypeptide/ATP1B3 | | | |
| | | | arginine vasopressin receptor 2 (nephrogenic | 304800 | NM_000054 | Xq28 |
| | | | diabetes insipidus)/AVPR2 | | | |
| | | | aquaporin 1/AQP1 | 107776 | NM_000385 | 7p14 |
| | | | aquaporin 2/AQP2 | 107777 | NM_000486 | 12q13 |
| | | | aquaporin 3/AQP3 | 600170 | NM_004925 | 9p113 |
| | | | aquaporin 6/AQP6 | 601383 | NM_001652 | 12q13 |
| | | Superoxide | Superoxide Dismutase 1/SOD1 (soluble) | 147450 | NM_000454 | 21q22.1 |
| | | Dismutase | Superoxide Dismutase 2/SOD2 (mitochondrial) | 147460 | X65965 | 6q25.3 |
| | | | Superoxide Dismutase 3/SOD3 (extracellular) | 185490 | NM_003102 | 4pter-q21 |
| Organ and | Protection from | Aldehyde | aldehyde dehydrogenase 1/ALDH1 (liver cytosol) | 100640 | AH002598 | 9q21 |
| Tissue | Radical Damage | Dehydrogenase | aldehyde dehydrogenase 2/ALDH2 (liver | 100650 | K03001 | 12q24.2 |
| Damage | | | mitochondria) | | | |
| | | | aldehyde dehydrogenase 3/acetaldehyde | 100660 | M74542 | 17p11.2 |
| | | | dehydrogenase/ALDH3 (stomach) | | | |
| | | | aldehyde dehydrogenase 5/acetaldehyde | 100670 | NM_000692 | 9p13 |
| | | | dehydrogenase/ALDH5 | | | |
| | | | aldehyde dehydrogenase 5, member Al/succinic | 271980 | NM_001080 | 6p22 |
| | | | semialdehyde dehydrogenase/ALDHSA1 | | | |
| | | | aldehyde dehydrogenase 6/acetaldehyde | 600463 | NM_000693 | 15q26 |
| | | | dehydrogenase/ALDH6 | | | |
| | | | aldehyde dehydrogenase 7/acetaldehyde | 600466 | NM_000694 | 11q13 |
| | | | dehydrogenase/ALDH7 | | | |
| | | | aldehyde dehydrogenase 8/ALDH8 | 601917 | NM_000695 | chr. 11 |
| | | | aldehyde dehydrogenase 9/g-aminobutyraldehyde | 602733 | NM_000696 | 1q22-q23 |
| | | | dehydrogenase/ALDH9 | | | |
| | | | aldehyde dehydrogenase 10/ALDH10 | 270200 | NM_000382 | 17p11.2 |
| | | Glutathione | glutathione synthetase/GSS | 601002 | NM_0001781 | 20q11.2 |
| | | | glutathione peroxidase/GPX1 | 138320 | M21304 | 3p21.3 |
| | | | glutathione peroxidase GPX2 | 138319 | X68314 | 14g24.1: |
| | | | glutathione peroxidase GPX3 | 138321 | X58295 | 5q32- |
| | | | | | | q33.1 |
| | | | glutathione peroxidase GPX4 | 138322 | X71973 | 19p13.3 |
| | | | glutathione peroxidase GPX5 | 603435 | AJ005277 | ****** |
| | | | glutathione reductase | 138300 | X15722 | 8p21.1 |
| | | Metallothionein | metallothionein 1A/MT1A | 156350 | NM_005953 | 16q13 |
| | | | metallothionein 1B | 56349 | AH001510 | 16q13 |
| | | | metallothionein 1E | 156351 | M10942 | 16q13 |
| | | | metallothionein 1F | 156352 | M10943 | 16q13 |
| | | | metallothionein 1G | 156353 | J03910 | 16q13 |
| | | | metallothionein 2A/MT2A | 156360 | NM_005953 | 16q13 |
| | | | metallothionein 3 | 139255 | NM_005954 | 16q13 |
| | | Miscellaneous | glucose-6-phosphate dehydrogenase/G6PD | 305900 | NM_000402 | Xq28 |
| | | | (mitochondrial) | | | |
| | | | 8-oxoguanine DNA glycosylase/OGG1 | 601982 | NM_002542 | 3p26.2 |
| | | | Peptide methionine sulfoxide reductase/MSRA | 601250 | ****** | ****** |
| | | | succinate dehydrogenase complex, subunit C, | 602413 | NM_003001 | 1q21 |
| | | | integral membrane protein/SDHC | | | |
| | | | phospholipase A2 group IB/PLA2G1B | 172410 | NM_000928 | 12q23- |
| | | | | | | q24.1 |
| | | | lipoprotein, Lp(a)/LPA | 152200 | NM_005577 | 6g27 |
| | | | Catalase/CAT | 115500 | NM_001752 | 11p13 |
| | | | thioredoxin-dependent peroxide reductase/TDPX1 | 600538 | NM_005809 | 13q12 |
| Immune | Mast Cell and T- | IgE Production | interleukin 4 receptor/IL4R | 147781 | X52425 | 16p12.1- |
| Response | Cell Response | | | | | p11.2 |
| | | | interferon gamma/IFNG | 147570 | L07633 | 12q14 |
| | | | mast cell growth factor/MGF | 184745 | NM_003994 | 12q22 |
| | | Mast Cell | interleukin 9 receptor/IL9R | 300007 | M84747 | Xq28 |
| | | Proliferation | interleukin 3 receptor/IL3R) | 308385 | M74782 | Xp22.3 |
| | | Degranulation | mast cell IgE receptor alpha polypeptide/FCER1A | 147140 | ****** | 1q23 |
| | | Mast Cells | mast cell IgE receptor beta polypeptide/FCER1B | 147138 | NM_000139 | 11q13 |
| | | | mast cell IgE receptor beta polypeptide/FCER1G | 147139 | NM_004106 | 1q23 |
| | | | SH2-containing inositol 5-phosphatase/SHIP | 601582 | U57650 | 2q36-q37 |
| | | | secretory granule proteoglycan peptide core/PRG1 | 177040 | J03223 | 10q22.1 |
| | | Histamine | Histidine Decarboxylase | 142704 | M60445 | 15q21- |
| | | | | | | q22 |
| | | | Histamine receptor H1 | 600167 | AF026261 | 3p21-p14 |
| | | | Histamine receptor H2 | 142703 | M64799 | ****** |
| | | | Histamine N-methyltransferase | ****** | D16224 | dir. 2 |
| | | | Amine oxidase (copper-containing) 2/AOC2 | 602268 | D88213 | 17q21 |
| | | | Amine oxidase (copper-containing) 3/AOC3 | 603735 | AF054985 | 17q21 |
| | | Serotonin | aromatic L-Amino Acid Decarboxylase/AADC | 107930 | M76180 | 7p11 |
| | | | tryptophan hydroxylase/TPH | 191060 | X52836 | 11p15.3- |
| | | | | | | p14 |
| | | | 14-3-3 protein ETA | 113508 | X78138 | 22q12 |
| | | | 14-3-3 protein ZETA | 601288 | M86400 | 2q25.2- |
| | | | | | | p25.1 |
| | | | 14-3-3 protein BETA | 601289 | X57346 | 20q13.1 |
| | | | 14-3-3 protein SIGMA | 601290 | X57348 | ****** |
| | | | serotonin 5-HT receptors 5-HT1A, G protein-coupled | 109760 | X57829 | 5q11.2- |
| | | | | | | q13 |
| | | | serotonin 5-HT receptors 5-HT1B, G protein-coupled | 182131 | M81590 | 6q13 |
| | | | serotonin 5-HT receptors 5-HT1C, G protein-coupled | 312861 | U49516 | Xq24 |
| | | | serotonin 5-HT receptors 5-HT1D, G protein-coupled | 182133 | M81590 | 1p36.3- |
| | | | | | | p34.3 |
| | | | serotonin 5-HT receptors 5-HT1E, G protein-coupled | 182132 | M91467 | 6q14-q15 |
| | | | serotonin 5-HT receptors 5-HT1F, G protein-coupled | 182134 | L05597 | 3p12 |
| | | | serotonin 5-HT receptors 5-HT2A, G protein-coupled | 182135 | D87030 | 13q14- |
| | | | | | | q21 |
| | | | scrotonin 5-HT receptors 5-HT2B, G protein-coupled | 601122 | X77307 | 2q36.3- |
| | | | | | | q37.1 |
| | | | serotonin 5-HT receptors 5-HT2C, G protein-coupled | 312861 | U49516 | Xq24 |
| | | | serotonin transporter | 182138 | X70697 | 17q11.1- |
| | | | | | | q12 |
| | | | monoamine oxidase A/MAOA | 309850 | M69226 | Xp11.23 |
| | | | monoamine oxidase B MAOB | 309860 | M69177 | Xp11.23 |
| | | | serotonin N-Acetyltransferase/SNAT | 600950 | U40347 | 17q25 |
| | | | tryptophan 2,3-dioxygenase/TDO2 | 191070 | NM_00565 | 14q31-q32 |
| | | Neutrophil and | eotaxin precursor/small inducible cytokine, family A, | 601156 | U46572 | 17q21.1- |
| | | Eosinophil | member 11/SCYA11 | | | q21.2 |
| | | Chemotaxis | monocyte-derived-neutrophil chemotactic | 146930 | M26383 | 4q12-q13 |
| | | | factor/interleukin 8/1L8 | | | |
| | | Proteases | tryptase alpha/TPS1 | 191080 | NM_003293 | Chr. 16 |
| | | | tryptase beta/TPS2 | 191081 | NM_003294 | Chr. 16 |
| | | | chymase 1, mast cell/CMA1 | 118938 | NM_001836 | 14g11.2 |
| | | Release of | phospholipase A2 group IIA/PLA2G2A | 172411 | NM_000300 | 1p35 |
| | | Membrane | phospholipase A2 group IB/PLA2G1B | 172410 | NM_000928 | 12q23- |
| | | Lipids | | | | q24.1 |
| | | (common to, | phospholipase A2 group X/PLA2G10 | 603603 | ****** | 16p13.1- |
| | | leukotriene, and | | | | p12 |
| | | prostaglandin | phospholipase A2 group IVA/PLA2G4A | 600522 | U08374 | 1q25 |
| | | pathways | phospholipase A2 group VI/PLA2G6 | 603604 | AF064594 | 22q13.1 |
| | | | phospholipase A2 group IVC/PLA2G4C | 603602 | ****** | chr. 19 |
| | | | phosholipase A2 group IVC/PLA2G4C | 603602 | ****** | chr. 19 |
| | | | phospholipase A2 group V/PLA2G5 | 601192 | NM_000929 | 1p36-p34 |
| | | | phospholipase C beta 3 | 600230 | U26425 | 11q13 |
| | | | lysosomal acid lipase | 278000 | NM_000235 | 10q24- |
| | | | | | | q25 |
| | | Platelet | CDP-choline: alkylacetylglycerol | ****** | ****** | ****** |
| | | Activating | cholinephosphotransferase | | | |
| | | Factor (PAF) | platelet activating factor receptor/PTAFR | 173393 | M88177 | 1p35- |
| | | | | | | p34.3 |
| | | | platelet activating factor acetylhydrolase 1/PAFAH1 | 601690 | NM_005084 | 6p21.2- |
| | | | | | | p12 |
| | | | platelet activating factor acetylhydrolase, isoform | 601545 | NM_000430 | 17p13.13 |
| | | | 1B, alpha subunit/PAFAH1B1 | | | |
| | | | platelet activating factor acetylhydrolase, isoform | 602508 | NM_002572 | 11q23 |
| | | | 1B, beta subunit/PAFAH1B2 | | | |
| | | | platelet activating factor acetylhydrolase, isoform | 603074 | NM_002573 | 19q13.1 |
| | | | 1B, gamma subunit/PAFAH1B3 | | | |
| | | | platelet activating factor acetylhydrolase 2/PAFAH2 | 602344 | NM_000437 | ****** |
| | | Leukotriene | arachidonate 5-lipoxygenase/ALOX5 | 152390 | NM_000698 | Chr.10 |
| | | | arachidonate 5-lipoxygenase-activating | 603700 | NM_001629 | 13q12 |
| | | | protein/FLAP/ALOX5AP | | | |
| | | | leukotriene A4 hydrolase/LTA4H | 151570 | NM_000895 | 12q22 |
| | | | leukotriene C4 synthase/LTC4S | 246530 | NM_000897 | 5q35 |
| | | | Gamma-glutamyltranspeptidase 1/GGT1 | 231950 | J04131 | 22q11.1- |
| | | | | | | q11.2 |
| | | | Gamma-glutamyltranspeptidase 2/GGT2 | 137181 | AH002728 | 22q11.1 |
| | | | Gamma-glutamyltransferase-like activity 1/GGTLA1 | 137168 | NM_004121 | ****** |
| | | | renal microsomal dipeptidase/DPEP1 | 179780 | NM_004413 | 16q24.3 |
| | | | cysteinyl leukotriene receptor 1/CYSLT1 | 300201 | NM_006639 | Xg13-g21 |
| | | | leukotriene b4 receptor (chemokine receptor-like | 601531 | NM_000752 | 14q11.2- |
| | | | 1)/LTB4R | | | q12 |
| | | Prostaglandins | prostaglandin endoperoxide synthetase | 176805 | AH001520 | 9q32- |
| | | | 1/COX1/PTGS1 | | | q33.3 |
| | | | prostaglandin endoperoxide synthetase | 600262 | NM_000963 | 1q25.2- |
| | | | 2/COX2/PTGS2 | | | q25.3 |
| | | | thromboxane A synthase 1/TBXAS1 | 274180 | SEG_D3461 | 7q34 |
| | | | | | 3S | |
| | | | prostaglandin D2 synthase | 602598 | M61900 | ****** |
| | | | prostaglandin I2 synthase/prostacyclin | 601699 | SEG_D8339 | 20q13 |
| | | | synthase/PTGIS | | 3S | |
| | | | prostaglandin E receptor 1, EP1 subtype/PTGER1 | 176802 | NM_000955 | 19p13.1 |
| | | | prostaglandin E receptor 2, EP2 subtype/PTGER2 | 176804 | ****** | 5p13.1 |
| | | | prostaglandin E receptor 3, EP3 subtype/PTGER3 | 176806 | NM_000957 | 1p31.2 |
| | | | prostaglandin E receptor 4, EP4 subtype/PTGER4 | 601586 | NM_000958 | 5p13.1 |
| | | | prostaglandin F receptor/PTGFR | 600563 | L24470 | 1p31.1 |
| | | | prostaglandin F2 receptor negative | 601204 | U26664 | 1p13.1- |
| | | | regulator/PTGFRN | | | q21.3 |
| | | | prostaglandin 12 receptor/PTGIR/prostacyclin | 600022 | SEG_HUMI | 19q13.3 |
| | | | receptor | | P | |
| | | | 15-hydroxyprostaglandin dehydrogenase/HPGD | 601688 | NM_000860 | 4q34-q35 |
| | | | aldo-keto reductase family 1, member C2/AKR1C2 | 600450 | NM_001353 | 10p15- |
| | | | | | | p14 |
| | | Formation of | myeloperoxidase/MPO | 254600 | J02694 | 17g23.1 |
| | | Reactive Drug | eosinophil peroxidase/EPX | 131399 | NM_000502 | ****** |
| | | Metabolites | calreticulin/CALR | 109091 | CALR | 19p13.2 |
| | | | calnexin/CANX | 114217 | L18887 | 5q35 |
| | | | ceruloplasmin (ferroxidase)/CP | 117700 | NM_000096 | 3q21-q24 |
| | | Antigen | MHC class II transactivator/MHC2TA | 600005 | NM_000246 | 16p13 |
| | | Presentation | MHC class II HLA DR-alpha chain/HLA-DRA | 142860 | X83114 | 6p21.3 |
| | | | MHC class II HLA DR-beta chain/HLA-DRB | 142857 | M11161 | 6p21.3 |
| | | | MHC class II HLA DP-alpha chain/HLA-DPA | 142880 | M23905 | 6p21.3 |
| | | | MHC class II HLA DP-beta chain/HLA-DPB | 142858 | AH002893 | 6p21.3 |
| | | | MHC class II ULA DM-alpha chain/HLA-DMA | 142855 | NM_006120 | 6p21.3 |
| | | | MHC class II HLA DM-beta chain/HLA-DMB | 142856 | NM_002118 | 6p21.3 |
| | | | MHC class II HLA DQ-alpha chain/HLA-DQA | 146880 | M11124 | 6p21.3 |
| | | | MHC class II HLA DQ-beta chain/HLA-DQB | ****** | M24364 | 6p21.3 |
| | | | MHC class II HLA DN-alpha chain/HLA-DNA | 142930 | X02882 | 6p21.3 |
| | | | MHC class II HLA DO-beta chain/HLA-DOB | 600629 | NM_002120 | 6p21.3 |
| | | | MHC class II antigen gamma chain/CD74 | 142790 | K01144 | 5g32 |
| | | | antigen peptide transporter 1/MHC 1/TAP1 | 170260 | NM_000593 | 6p21.3 |
| | | | antigen peptide transporter 2/MHC 2/TAP2 | 170261 | NM_000544 | 6p21.3 |
| | | T-Cell Receptor | T-cell antigen receptor, alpha subunit/TCRA | 186880 | Z24457 | 14q11.2 |
| | | | T-cell antigen receptor, beta subunit/TCRB | 186930 | AF011643 | 7q35 |
| | | | T-cell antigen receptor, gamma subunit/TCRG | 186970 | M17325 | 7p15-p14 |
| | | | T-cell antigen receptor, delta subunit/TCRD | 186810 | L36384 | 14q11.2 |
| | | | thymocyte antigen receptor complex CD3G, gamma | 186740 | NM_000073 | 11q23 |
| | | | polypeptide (TiT3 complex)/CD3G | | | |
| | | | thymocyte antigen receptor complex CD3D, delta | 186790 | NM_000732 | 11q23 |
| | | | polypeptide (TiT3 complex)/CD3D | | | |
| | | | thymocyte antigen receptor complex CD3E, epsilon | 186830 | NM_000733 | 11q23 |
| | | | polypeptide (TiT3 complex)/CD3E | | | |
| | | | thymocyte antigen receptor complex CD3Z, zeta | 186780 | NM_000734 | 1q22-q23 |
| | | | polypeptide (TiT3 complex)/CD3Z | | | |
| | | T-Cell Receptor | ataxia telangiectasia mutated (includes | 208900 | NM_000051 | 11q22.3 |
| | | Rearrangement | complementation groups A, C and D)/ATM | | | |
| | | | recombination activating gene 1/RAG1 | 179615 | NM_000448 | 11p13 |
| | | | recombination activating gene 2/RAG2 | 179616 | M94633 | 11p13 |
| | | | interleukin 7 receptor/IL7R | 146661 | NM_002185 | 5p13 |
| | | | v-myb avian myeloblastosis viral oncogene | 189990 | NM_005375 | 6q22 |
| | | | homolog/MYB | | | |
| | | | core binding factor, alpha 1 subunit/CBFA1 | 600211 | AH005498 | 6p21 |
| | | | core-binding factor, beta subunit/PEBP2B/CBFB | 121360 | L20298 | 16q22 |
| | | | ligase I, DNA, ATP-dependent/LIG1 | 126391 | NM_000234 | 19q13.2- |
| | | | | | | q13.3 |
| | | | ligase IV, DNA, ATP-dependent/LIG4 | 601837 | NM_002312 | 13q22- |
| | | | | | | q34 |
| | | | X-ray repair, complementing defect in Chinese | 194364 | ****** | 2q35 |
| | | | hamster/Ku antigen, 80 kD/KU80/XRCC5 | | | |
| | | | thyroid autoantigen, 70 kD/KU70/G22P1 | 152690 | NM_001469 | 22q11- |
| | | T-Cell | | | | q13 |
| | | Expansion | T-cell antigen T4/CD4 | 186940 | X87579 | 12pter- |
| | | | | | | p12 |
| | | | T-cell antigen CD8, alpha polypeptide (p32)/CD8A | 186910 | NM_001768 | 2p12 |
| | | | T-cell antigen CD8, beta polypeptide/CD8B | 186730 | AH003859 | 2p12 |
| | | | T-cell antigen CD28 (Tp44)/CD28 | 186760 | NM_006139 | 2q33-q34 |
| | | | cytotoxic T-lymphocyte-associated 4/CTLA4 | 123890 | L15006 | 2q33 |
| | | | CD80 antigen (CD28 antigen ligand 1, B7-1 | 112203 | NM_005191 | 3q21 |
| | | | antigen)/CD80 | | | |
| | | | CD86 antigen (CD28 antigen ligand 2, B7-2 | 601020 | NM_006889 | 3q21 |
| | | | antigen)/CD86 | | | |
| | | | T cell receptor-associated protein tyrosine kinase | 176947 | S69911 | 2q12 |
| | | | ZAP-70/ZAP70 | | | |
| | | | leukocyte common antigen T200/CD45 | 151460 | M23492 | 1q31-q32 |
| | | | nuclear factor of activated T-cells, cytoplasmic | 600489 | NM_006162 | 18q23 |
| | | | 1/NFATC1 | | | |
| | | | nuclear factor of activated T-cells, cytoplasmic | 600490 | ****** | 20q13.2- |
| | | | 2/NFATC2 | | | q13.3 |
| | | | nuclear factor of activated T-cells, cytoplasmic | 602698 | L41066 | 16q13- |
| | | | 3/NFATC3 | | | q24 |
| | | | nuclear factor of activated T-cells, cytoplasmic | 602699 | L41067 | ****** |
| | | | 4/NFATC4 | | | |
| | | | interleukin 2 receptor alpha/IL2RA | 147730 | X01057 | 10p15- |
| | | | | | | p14 |
| | | | interleukin receptor beta/IL2RLB | 146710 | M26062 | 22q11.2- |
| | | | | | | q13 |
| | | | interleukin 2 receptor gamma/IL2RG | 308380 | D11086 | Xq13 |
| | | | interleukin 6 receptor/IL6R | 147880 | X12830 | 1q21 |
| | | | interleukin 9 receptor/IL9R | 300007 | M84747 | Xq28 |
| | | | interleukin receptor 13 alpha/IL13RA1 | 300119 | S80963 | Chr.X |
| | | | interleukin receptor 13 alpha2/1L13RA2 | 300130 | X95302 | Xq24 |
| | | | interleukin 15 receptor alpha/IL15RA | 601070 | U31628 | 10p15- |
| | | | | | | p14 |
| | | | transforming growth factor/TGFB1 | 190180 | M60315 | 19q13.1- |
| | | | | | | q13.3 |
| | | | transforming growth factor/TGFB2 | 190220 | M19154 | 1q41 |
| | | | transforming growth factor/TGFB3 | 190230 | X14149 | 14q24 |
| | | | tumor necrosis factor beta/TNFB/lymphotoxin | 153440 | NM_000595 | 6p21.3 |
| | | | alpha/LTA | | | |
| | | | tumor necrosis factor ligand superfamily, member | 134638 | NM_000639 | 1q23 |
| | | | 6/TNFSF6 | | | |
| | | | tumor necrosis factor receptor superfamily, member | 134637 | NM_000043 | 10q24.1 |
| | | | 6/TNFRSF6 | | | |
| | | | caspase 10, apoptosis-related cysteine | 601762 | NM_001230 | 2q33-q34 |
| | | | protease/CASP10 | | | |
| | B-Cell Response | Receptors | B-cell antigen CD20/B-lymphocyte differentiation | 112210 | AH003353 | 11q13 |
| | | | antigen B1/CD20 | | | |
| | | | B-cell antigen CD72/CD72 | 107272 | | 9p |
| | | | | | NM_001782 | |
| | | | natural resistance-associated macrophage protein | 600266 | AH002806 | 2q35 |
| | | | 1/NRAMP1/solute carrier family 11, member | | | |
| | | | 1/SLC11A1 | | | |
| | | | natural resistance-associated macrophage protein | 600523 | AB015355 | 12q13 |
| | | | 2/NIRAMP2/solute carrier family 11, member | | | |
| | | | 1/SLC11A2 | | | |
| | | | T-lymphocyte antigen CDW52 (CAMPATH-1 | 114280 | NM_001803 | ****** |
| | | | antigen)/CDW52 | | | |
| | | | B-cell antigen CD22/CD22 | 107266 | NM_001771 | 19q13.1 |
| | | | B-cell antigen CD24/CD62 ligand/CD24 | 600074 | X69397 | 6q21 |
| | | | leukocyte antigen CD156/disintegrin and | 602267 | NM_001109 | 10q26.3 |
| | | | metalloprotease domain 8/ADAM8/CD156 | | | |
| | | | platelet antigen CD151/platelet-endothelial cell | 602243 | NM_004357 | 11p15.5 |
| | | | tetraspan antigen 3/PETA3/CD151 | | | |
| | | | antigen CD32/low-affinity receptor IIA for Fc | 146790 | NM_004001 | 1q21-q23 |
| | | | fragment of IgG/FCGR2A/CD32 | | | |
| | | | activated leucocyte cell adhesion molecule/CD6 | 601662 | NM_001627 | 3q13.1- |
| | | | ligand/ALCAM | | | q13.2 |
| | | | lymphocyte antigen CD79A/immunoglobulin- | 112205 | NM_001783 | 19q13.2 |
| | | | associated alpha/CD79A | | | |
| | | | lymphocyte antigen CD79B/immunoglobulin- | 147245 | L27587 | 17q23 |
| | | | associated beta/CD79B | | | |
| | | Signalling | regulator of G-protein signalling 1/RGS1 | 600323 | NM_002922 | 1q31 |
| | | Immunoglobulin | immunoglobulin K light chain constant region | 147200 | ****** | 2p12 |
| | | Light Chains | locus/IGKC | | | |
| | | | immunoglobulin K light chain variable region | 146980 | K01322 | 2p12 |
| | | | locus/IGKV | | | |
| | | | immunoglobulin K light chain joining region | 146970 | ****** | 2p12 |
| | | | locus/IGKJ | | | |
| | | | immunoglobulin L light chain constant region | 147220 | NM_006146 | 22q11.2 |
| | | | locus/IGLC1 | | | |
| | | | immunoglobulin L light chainjoining region | 147230 | NM_006146 | 22q11.2 |
| | | | locus/IGLJ | | | |
| | | | immunoglobulin L light chain variable region | 147240 | NM_006146 | 22q11.2 |
| | | | locus/IGLJ | | | |
| | | Immunoglobulin | immunoglobulin A heavy chain constant region locus | 146900 | ****** | 14q32.33 |
| | | Heavy Chains | 1/IGHA1 | | | |
| | | | immunoglobulin A heavy chain constant region locus | 147000 | ****** | 14q32.33 |
| | | | 2/IGHA2 | | | |
| | | | immunoglobulin D heavy chain constant region | 147170 | ****** | 14q32.33 |
| | | | locus/IGHD | | | |
| | | | immunoglobulin B heavy chain constant region | 147180 | ****** | 14q32.33 |
| | | | locus/IGHE | | | |
| | | | immunoglobulin G heavy chain constant region locus | 147100 | ****** | 14q32.33 |
| | | | 1/IGHG1 | | | |
| | | | immunoglobulin G heavy chain constant region locus | 147110 | ****** | 14q32.33 |
| | | | 2/IGHG2 | | | |
| | | | immunoglobulin G heavy chain constant region locus | 147120 | ****** | 14q32.33 |
| | | | 3/IGHG3 | | | |
| | | | immunoglobulin G heavy chain constant region locus | 147130 | ****** | 14q32.33 |
| | | | 4/IGHG4 | | | |
| | | | immunoglobulin M heavy chain constant region | 1147020 | ****** | 14q32.33 |
| | | | locus/IGHM | | | |
| | | | immunoglobulin heavy chain variable region locus | 147070 | X92279 | 14q32.33 |
| | | | 1/IGHV1 | | | |
| | | | immunoglobulin heavy chain variable region locus | 600949 | ****** | 16p11 |
| | | | 2/IGHV2 | | | |
| | | | immunoglobulin heavy chain diversity region locus | 146910 | X97051 | 14q32.33 |
| | | | 1/IGHDY1 | | | |
| | | | immunoglobulin heavy chain diversity region locus | 146990 | L25544 | 15q11- |
| | | | 2/IGHDY2 | | | q12 |
| | | | immunoglobulin heavy chain joining region | 147010 | ****** | 14q32.33 |
| | | | locus/IGHJ | | | |
| | | Immunoglobulin | recombination activating gene 1/RAG1 | 179615 | NM_000448 | 11p13 |
| | | Gene | recombination activating gene 2/RAG2 | 179616 | M94633 | 11p13 |
| | | Rearrangement | immunoglobulin kappa J region recombination signal | 147183 | L07872 | 9p13-p12 |
| | | | binding protein/RBPJK/IGKJRB1 | | | |
| | | | Bruton agammaglobulinemia tyrosine kinase/BTK | 300300 | NM_000061 | Xq21.3- |
| | | | | | | q22 |
| | | | interleukin 7 receptor/IL7R | 146661 | NM_002185 | 5p13 |
| | | | interferon-gamma receptor 1/IFNGR1 | 107470 | NM_000416 | 6q23-q24 |
| | | | interferon-gamma receptor 2/IFNGR2 | 147569 | NM_005534 | 21q22.1- |
| | | | | | | q22.2 |
| | | | interleukin 4 receptor precursor/IL4R | 147781 | NM_000418 | 16p12.1- |
| | | | | | | p11.2 |
| | | | interleukin 4 receptor precursor/IL4R | 147781 | NM_000418 | 16p12.1- |
| | | | | | | 11.2 |
| | | | ligase I, DNA, ATP-dependent/LJG1 | 126391 | NM_000234 | 19q13.2- |
| | | | | | | q13.3 |
| | | | ligase IV, DNA, ATP-dependent/LIG4 | 601837 | NM_002312 | 13q22- |
| | | | | | | q34 |
| | | | X-ray repair, complementing defect in Chinese | 194364 | ****** | 2q35 |
| | | | hamster/Ku antigen, 80 kD/KU80/XRCC5 | | | |
| | | | thyroid autoantigen, 70 kD/KU70/G22P1 | 152690 | NM_001469 | 22q11- |
| | | | | | q13 | |
| | | Immunoglobulin | nuclear factor kappa-B DNA binding subunit | 164011 | M58603 | 4q23-q24 |
| | | Gene | 1/NFKB1 | | | |
| | | Transcription | nuclear factor kappa-B DNA binding subunit | 164012 | NM_002502 | 10q24 |
| | | | 2/NFKB2 | | | |
| | | | nuclear factor kappa-B subunit 3/NFKB3 | 164014 | Z22949 | 11q12- |
| | | | | | q13 | |
| | | | nuclear factor of kappa light chain gene enhancer in | 164008 | ****** | 14q13 |
| | | | B cells, inhibitor alpha/NFKIBIA | | | |
| | | | nuclear factor of kappa light chain gene enhancer in | 603258 | NM_002503 | 8p11.2 |
| | | | B cells, inhibitor beta/NFKBIB | | | |
| | | | YY1 transcription factor/YY1 | 600013 | NM_003403 | 14q |
| | | | immunoglobulin transcription factor | 147141 | ****** | 19p13.3 |
| | | | 1/ITF1/transcription factor 3/TCF3 | | | |
| | | | immunoglobulin transcription factor | 602272 | NM_003199 | 18q21.1 |
| | | | 2/ITF2/transcription factor 4/TCF4 | | | |
| | | | immunoglobulin mu binding protein 2/IGHMBP2 | 600502 | NM_002180 11q13.2- | |
| | | | | | | q13.4 |
| | | | transcription factor binding to IGHM enhancer | 314310 | NM_006521 | Xp11.22 |
| | | | 3/TFE3 | | | |
| | | | homeobox protein OCT1/POU domain transcription | 164175 | NM_002697 | 1q22-q23 |
| | | | factor 2,class 1/POU2F1 | | | |
| | | | homeobox protein OCT2/POU domain transcription | 164176 | M22596 | Chr.19 |
| | | | factor 2,class 2/POU2F2 | | | |
| | | | POU domain, class 2, associating factor 1/POU2AF1 | 601206 | NM_006235 | 11q23.1 |
| | | | inhibitor of DNA binding 1, dominant negative helix- | 600349 | NM_002165 | 20q11 |
| | | | loop-helix protein/ID1 | | | |
| | | | inhibitor of DNA binding 2, dominant negative helix- | 600386 | NM_002166 | 2p25 |
| | | | loop-helix protein/1D2 | | | |
| | | Immunoglobulin | B-cell antigen CD40/tumor necrosis factor receptor | 109535 | NM_001250 | 20q12- |
| | | Isotype | superfamily, member 5/CD40/TNFRSFS | | | q13.2 |
| | | Switching | paired box gene 5/B-cell lineage-specific activator | 167414 | ****** | 9p13 |
| | | | protein/BSAP/PAX5 | | | |
| | | | lymphocye function-associated antigen, type | 153420 | NM_001779 | 1p13 |
| | | | 3/LFA3/LEU7/CD58 | | | |
| | | | interleukin 10 receptor, alpha/IL10RA | 146933 | NM_001558 | 11q23.3 |
| | | | lymphocyte antigen CD45/protein tyrosine | 151460 | NM_002838 | 1q31-q32 |
| | | | phosphatase, receptor type, c | | | |
| | | | polypeptide/PTPRC/CD45 | | | |
| | | | prostaglandin E receptor 1, EP1 subtype/PTGER1 | 176802 | NM_000955 | 19p13.1 |
| | | | prostaglandin E receptor 2, EP2 subtype/PTGER2 | 176804 | ****** | 5p13.1 |
| | | | prostaglandin E receptor 3, EP3 subtype/PTGER3 | 176806 | NM_000957 | 1p31.2 |
| | | | prostaglandin E receptor 4, EP4 subtype/PTGER4 | 601586 | NM_000958 | 5p13.1 |
| | | | interleukin 13 receptor, alpha 1/IL13RA1 | 300119 | NM_001560 | Chr.X |
| | | | interleukin receptor 13 alpha2/IL13A2 | 300130 | X95302 | Xq24 |
| | | | interferon-gamma receptor 1/IFNGR1 | 107470 | NM_000416 | 6q23-q24 |
| | | | interferon-gamma receptor 2/IFNGR2 | 147569 | NM_005534 | 21q22.1- |
| | | | | | | q22.2 |
| | | | interleukin 5 receptor alpha/IL5RA | 147851 | M96652 | 3p26-p24: |
| | | | transforming growth factor, beta receptor I (activin A | 190181 | NM_004612 | 9q33-q34 |
| | | | receptor type II-like kinase, 53kD)/TGFBR1 | | | |
| | | | transforming growth factor, beta receptor II(70- | 190182 | NM_003242 | 3p22 |
| | | | 80kD)/TGFBR2 | | | |
| | | | transforming growth factor, beta receptor III | 600742 | NM_003243 | 1p33-p32 |
| | | | (betaglycan, 300kD)/TGFBR3 | | | |
| | | | X-ray repair, complementing defect in Chinese | 194364 | ****** | 2q35 |
| | | | hamster/Ku antigen, 80 kD/KU80/XRCC5 | | | |
| | | | thyroid autoantigen, 70 kD/KU70/G22P1 | 152690 | NM_001469 | 22q11- |
| | | | | | | q13 |
| | Myeloid | Granulocyte, | granulocyte-macrophage colony stimulating factor | 138960 | NM_000758 | 5q31.1 |
| | Differentiation | Macrophage, | 2/CSF2 | | | |
| | | Erythrocyte, | macrophage-specific colony-stimulating factor/CSF | 1120420 | AH005300 | 1p21-p13 |
| | | and Platelet | granulocyte colony stimulating factor 3/CSF3 | 138970 | NM_000759 | 17q11.2- |
| | | Differentiation | | | | q12 |
| | | | colony stimulating factor 1 receptor/CSFR1 | 164770 | U63963 | 5q33.2- |
| | | | | | | q33.3 |
| | | | granulocyte-macrophage colony stimulating factor 2 | 306250 | NM_006140 | Xp22.32 |
| | | | receptor, alpha, low-affinity/CSF2RA | | | |
| | | | granulocyte-macrophage colony stimulating factor 2 | 138981 | U18373 | 22q12.2- |
| | | | receptor, beta/CSF2RB | | | q13.1 |
| | | | granulocyte-macrophage colony stimulating factor 2 | 425000 | ****** | Yp11 |
| | | | receptor, alpha, Y chromasomal/CSF2RY | | | |
| | | | flt3 ligand/FMS-related tyrosine kinase 3 | 600007 | U03858 | 19q13.3 |
| | | | ligand/FLT3LG | | | |
| | | | STAT induced STAT inhibitor 3/SSI-3 | 604176 | NM_003955 | ****** |
| | | | erythropoietin/EPO | 133170 | NM_000799 | 7q21 |
| | | | erythropoietin receptor/EPOR | 133171 | NM_000121 | 19p13.3- |
| | | | | | | p13.2 |
| | | | Janus kinase 2 (a protein tyrosine kinase)/JAK2 | 147796 | NM_004972 | 9p24 |
| | | | STAM-like protein containing SH3 and ITAM | ****** | NM_005843 | ****** |
| | | | domains 2/STAM2 | | | |
| | | | ribosomal protein S7/RPS7 | 603474 | NM_001011 | 19g13.2 |
| | | | signal transducer and activator of transcription | 601511 | NM_003152 | 17q11.2 |
| | | | 5A/STAT5A | | | |
| | | | BCL-X/BCLX | 600039 | Z23115 | ****** |
| | | | thrombopoietin (MLV oncogene ligand, | 600044 | NM_000460 | 3q26.3- |
| | | | megakaryocyte growth and development | | | q27 |
| | | | factor)/THPO | | | |
| | | | mycloproliferative leukemia virus | 159530 | NM_005373 | 1p134 |
| | | | oncogene/MPL/thrombopoietin receptor/TPOR | | | |
| | | | FMS-related tyrosine kinase 3/FLT3 | 136351 | NM_004119 | 13q12 |

TABLE 2

[chemical structure]

[chemical structure]

TABLE 3

| | | | |
|---|---|---|---|
| Hugo | GID | OMIM ID VGX Symbol | Description |
| Variance Start | | Variance | |

| D89078 | | D89078 | | 601531 | | GEN-7 | | Leukotriene B4 receptor, cDNA | |
|---|---|---|---|---|---|---|---|---|---|
| | 434A | 545G | 889G | 1156G | 1393C | 1708A | 1715G | 1771T | 2644C |
| | 434A | 545G | 889G | 1156G | 1393C | 1708A | 1715T | 1771T | 2644C |
| | 434A | 545G | 889C | 1156G | 1393C | 1708A | 1715G | 1771T | 2644C |
| | 434A | 545G | 889G | 1156G | 1393T | 1708A | 1771T | 2644C | |
| | 434A | 545G | 889G | 1156G | 1393C | 1708C | 1715G | 1771T | 2644C |
| | 434A | 545G | 889C | 1156G | 1393C | 1705C | 1715G | 1771T | |
| | 434A | 545G | 889G | 1156G | 1393C | 1708C | 1715G | 1771T | 2644T |
| | 434A | 545G | 889G | 1156G | 1393C | 1708A | 1771C | 2644C | |
| | 434A | 545G | 889G | 1156G | 1393C | 1708A | 1715G | 1771T | 2644T |
| | 434A | 545A | 889G | 1156G | 1393C | 1715G | 1771T | 2644T | |
| | 434T | 889C | | | | | | | |
| | 434T | 545G | 889G | 1156G | 1393C | 1708A | 1715G | 1771T | |
| | 434A | 889G | 1156C | 1393C | 1708A | | | | |
| | 434A | 545G | 889C | 1156C | 1393C | 2644C | | | |
| | 434A | 545G | 889G | 1156G | 1393C | 1708A | 1715G | 1771C | 2644C |
| | 434A | 545G | 889G | 1156G | 1393T | 1708A | 1771T | 2644C | |
| | 434A | 545G | 889C | 1156G | 1393C | 1708C | 1715G | 1771T | 2644T |
| | 434A | 545G | 889G | 1156C | 1393C | 2644C | | | |
| | 434A | 889G | 1156C | 1393C | 1708A | 2644C | | | |
| | 434T | 545G | 889G | 1156G | 1393C | 1708A | 1715G | 1771T | 2644C |
| | 434T | 889C | 1156C | | | | | | |
| | 434A | 545G | 889G | 1156G | 1393C | 2644C | | | |
| | 434A | 545A | 889G | 1156G | 1393C | 1708C | 1715G | 1771T | 2644T |
| | 434A | 545G | 889C | 1156G | 1393C | 1708A | 1715T | 1771T | 2644C |

| | | |
|---|---|---|
| 434 | (−1284)A > T | 5′ |
| 545 | (−1173)G > A | 5′ |
| 889 | (−829)G > C | 5′ |
| 1156 | (−562)G > C | 5′ |
| 1393 | (−325)C > T | 5′ |
| 1708 | (−10)C > A | 5′ |
| 1715 | (−3)G > T | 5′ |
| 1771 | 54T > C | Silent |
| 2644 | 927T > C | Silent |
| 2920 | 1203A > G | 3′ |

| | | | | |
|---|---|---|---|---|
| J03459 | J03459 | 151570 | GEN-8 | Leukotriene A4 hydrolase |

| | | | | |
|---|---|---|---|---|
| 79T | 140G | 323G | 1511A | 1912C |
| 79T | 140G | 323A | 1511A | 1912C |
| 79C | 140G | 323G | 1511A | 1912C |
| 79T | 140T | 323G | 1511A | 1912C |
| 79T | 140G | 1912T | | |
| 140G | 323G | 1912C | | |
| 79T | 140G | 323A | 1511A | 1912T |

| | | |
|---|---|---|
| 79 | 11T > C | I4T |
| 140 | 72G > T | Silent |
| 323 | 255G > A | Silent |
| 1511 | 1443A > T | E481D |
| 1912 | 1844C > T | 3′ |

| | | | | |
|---|---|---|---|---|
| J03571 | J03571 | 152390 | GEN-9 | Lipoxygenases: 5-lipoxygenase |
| (leukocytes) | | | | |

| | |
|---|---|
| 304A | 959C |
| 304G | 959A |
| 304G | 959C |

| | | |
|---|---|---|
| 304 | 270G > A | Silent |
| 959 | 925C > A | P309T |
| 2076 | 2042-2043delAC | 3′ |
| 2328 | 2294C > T | 3′ |
| 2376 | 2342T > G | 3′ |

| | | | | |
|---|---|---|---|---|
| J05594 | J05594 | 601688 | GEN-E | Prostaglandin 15-OH |

dehydrogenase (PGDH)

| | | | | | | | | | | |
|---|---|---|---|---|---|---|---|---|---|---|
| 363T | 436C | 506C | 540G | 913C | 950G | 1236G | 1448A | 1780T | 1800A | 1972T |
| 363T | 436C | 506C | 540G | 913C | 1236T | 1448G | 1780C | 1800A | 1972T | |
| 363T | 436C | 506T | 540G | 913C | 950G | 1236G | 1448A | 1780T | 1800A | 1972T |
| 363T | 436C | 506C | 540G | 913C | 950A | 1236T | 1780T | 1800A | 1972T | |
| 363T | 436C | 506C | 540G | 913C | 950G | 1236G | 1448G | 1780T | 1800A | 1972C |
| 363T | 436C | 506C | 540A | 913C | 950G | 1236G | 1448G | 1780T | 1800A | 1972T |
| 363T | 436C | 506C | 540G | 913C | 950G | 1236T | 1780T | 1800A | 1972T | |
| 363C | 436C | 506C | 540G | 913C | 950G | 1236G | 1780T | 1800A | | |
| 363T | 436C | 506C | 540G | 913C | 950G | 1236G | 1448G | 1780T | 1800T | 1972T |
| 363T | 436C | 506C | 540G | 913C | 950G | 1236G | 1448G | 1780T | 1800A | 1972T |
| 363T | 436C | 506C | 540G | 913C | 1236T | 1780T | 1800T | 1972T | | |
| 363T | 436C | 506C | 540G | 913C | 950G | 1236G | 1448A | 1780T | 1800A | 1972C |
| 913C | 950G | 1236G | 1448G | 1780C | 1800A | 1972T | | | | |
| 363T | 436C | 506T | 540G | 913C | 950G | 1236G | 1448G | 1780T | 1800A | 1972T |
| 363T | 436C | 506C | 540G | 913G | 950G | 1236G | 1780T | 1800A | | |
| 363T | 436T | 506C | 540G | 913C | 950G | 1236G | 1780T | 1800A | 1972T | |
| 363T | 436C | 506C | 540G | 913C | 950G | 1236G | 1780T | 1800T | 1972C | |
| 363T | 436C | 506C | 540G | 913C | 950G | 1236T | 1448G | 1780T | 1800A | 1972T |
| 1236G | 1448G | 1780T | 1800A | 1972C | | | | | | |
| 363T | 436C | 506C | 540G | 913C | 950A | 1236T | 1448A | 1780T | 1800A | 1972T |
| 363T | 436C | 506C | 540G | 913C | 950A | 1236T | 1448A | 1780T | 1800T | 1972T |
| 1236G | 1448A | 1780T | 1800A | 1972T | | | | | | |
| 363T | 436C | 506C | 540G | 913C | 950A | 1236T | 1448G | 1780C | 1800A | 1972T |
| 913C | 950G | 1236G | 1448G | 1780C | 1800A | 1972T | | | | |
| 363T | 436C | 506C | 540G | 913C | 950G | 1236G | 1448G | 1780T | 1800T | 1972C |
| 363T | 436C | 506C | 540G | 913G | 950G | 1236G | 1448G | 1780T | 1800A | 1972C |
| 363T | 436C | 506C | 540G | 913C | 950G | 1236G | 1448A | 1780T | 1800T | 1972C |

| | | |
|---|---|---|
| 173 | 156A > G | Silent |
| 363 | 346T > C | Y116H |
| 436 | 419C > T | A140V |
| 506 | 489C > T | Silent |
| 540 | 523G > A | G175S |
| 913 | 896C > G | 3′ |
| 950 | 933G > A | 3′ |
| 1236 | 1219G > T | 3′ |
| 1448 | 1431G > A | 3′ |
| 1780 | 1763T > C | 3′ |
| 1800 | 1783A > T | 3′ |
| 1972 | 1955T > C | 3′ |

| L24470 | | L24470 | | 600563 | | GEN-O | | PROSTAGLANDIN F RECEPTOR |
|---|---|---|---|---|---|---|---|---|
| | 1422T | 1490T | 1517A | 1535G | 1908T | 2203C | 2244A | 2299A |
| | 1422C | 1490C | 1517A | 1535A | 1908C | 2203A | 2244A | 2299A |
| | 1422T | 1490T | 1517A | 1535A | 1908C | 2203A | 2244A | 2299A |
| | 1422T | 1490C | 1517A | 1908T | 2244A | 2299A | | |
| | 1422T | 15170 | 2244A | 2299A | | | | |
| | 1422T | 1490C | 1517A | 1535A | 1908C | 2203A | 2244A | 2299A |
| | 1422T | 1490C | 1517A | 1535A | 1908C | 2203A | 2244G | 2299A |
| | 1422T | 1517A | 1535A | 1908C | 2203A | 2244A | 2299G | |
| | 1422T | 149CC | 1517A | 1535G | 1908T | 2203C | 2244A | 2299A |
| | 1422T | 1490T | 15170 | 1535G | 1908T | 2203C | 2244A | 2299A |
| | 1422T | 1490T | 1517A | 2244A | 2299A | | | |
| | 1490C | 1517A | 1535A | 1908C | 2203A | 2244A | 2299A | |
| | 1422T | 1490T | 1517A | 1535A | 1908C | 2203A | 2244A | 2299G |
| | 1422T | 1490T | 1517A | 1535A | 1908T | 2203C | 2244A | 2299A |

| | | |
|---|---|---|
| 1422 | 1185T > C | 3′ |
| 1490 | 1253C > T | 3′ |
| 1517 | 1280A > G | 3′ |
| 1535 | 1298A > G | 3′ |
| 1908 | 1671C > T | 3′ |
| 2203 | 1966A > C | 3′ |
| 2244 | 2007A > G | 3′ |
| 2299 | 2062A > G | 3′ |

| | | | | |
|---|---|---|---|---|
| M12959 | M12959 | 186880 | GEN-S | CD3 glycoprotein on T |
| lymphocytes | | | | |

| | | |
|---|---|---|
| 431 | 295T > G | S99A |
| 1060 | 924T > C | 3′ |
| 1129 | 993C > A | 3′ |
| 1343 | 1207T > C | 3′ |
| 1345 | 1209G > C | 3′ |
| 1394 | 1258T > G | 3′ |
| 1463 | 1327G > A | 3′ |

| | | | | |
|---|---|---|---|---|
| M59979 | M59979 | 176805 | GEN-Z | Cyclooxygenase 1 COX1 |

| | | | | | | |
|---|---|---|---|---|---|---|
| 128G | 559A | 1517T | 1892A | | | |
| 128G | 559C | 644A | 1517T | 1892C | 2169T | 2296C |
| 128G | 559A | 644A | 1517T | 1892C | 2169T | 2296C |
| 128A | 559A | 644C | 1517T | 1892C | 2169T | 2296A |
| 128G | 559A | 1517C | 1892A | | | |
| 128G | 559A | 644C | 1517T | 1892C | 2169T | 2296A |
| 128G | 559A | 644A | 1517T | 1892C | 2169T | 2296A |
| 128G | 559A | 644C | 1517T | 1892C | 2169T | 2296C |
| 128G | 559A | 644C | 1517T | 1892A | 2169C | 2296A |
| 128G | 559A | 1517C | 1892A | 2169T | 2296C | |
| 644A | 1892C | | | | | |
| 128G | 559A | 644A | 1517C | 1892A | 2169C | 2296C |
| 128A | 559C | 644A | 1517T | 1892C | 2169T | 2296C |

| | | |
|---|---|---|
| 128 | 123G > A | Silent |
| 559 | 554A > C | K18ST |
| 644 | 639C > A | Silent |
| 1517 | 1512T > C | Silent |
| 1892 | 1887C > A | 3′ |
| 2030 | 2025G > A | 3′ |
| 2169 | 2164T > C | 3′ |
| 2296 | 2291A > C | 3′ |

| | | | | |
|---|---|---|---|---|
| M90100 | M90100 | 600262 | GEN-1A | Cyclooxygenase 2 COX2 |

| | | | |
|---|---|---|---|
| 2159G | 2339C | 2409A | 2983C |
| 2159G | 2339T | 2409G | 2983C |
| 2159C | 2339T | 2409G | |
| 2159G | 2339C | 2409G | 2983C |
| 2159G | 2409A | 2983C | |
| 2159C | 2339T | 2409G | 2983T |

| | | |
|---|---|---|
| 2159 | 2062G > C | 3′ |
| 2186 | 2089-2094delATATTA | 3′ |
| 2230 | 2133A > G | 3′ |
| 2339 | 2242T > C | 3′ |
| 2409 | 2312G > A | 3′ |
| 2726 | 2629C > T | 3′ |
| 2983 | 2886C > T | 3′ |

| | | | | |
|---|---|---|---|---|
| U49516 | 1349516 | 312861 | GEN-1Q | Serotonin 5-HT receptors 5-HT2C |

| | | | | | |
|---|---|---|---|---|---|
| 63C | 289A | 313G | 342A | 2915C | 2947A |
| 63C | 289G | 313A | 2915A | 2947A | |
| 63C | 289A | 313A | 342A | 2915A | 2947A |
| 63T | 289A | 313A | 342A | 2915A | 2947G |
| 63C | 289A | 313A | 342A | 2915C | 2947A |
| 63C | 289G | 313A | 342G | 2915A | 2947A |
| 63C | 289A | 313A | 342A | | |

| | | |
|---|---|---|
| 63 | (−666)C > T | 5′ |
| 289 | (−440)A > G | 5′ |
| 313 | (−416)A > G | 5′ |
| 342 | (−387)A > G | 5′ |
| 2915 | 2187A > C | 3′ |
| 2947 | 2219A > G | 3′ |

| | | | | |
|---|---|---|---|---|
| X06538 | X06538 | 180240 | GEN-1U | Retinoic Acid alpha receptor |

| | |
|---|---|
| 1063C | 1617T |
| 1063T | 1617C |
| 1063C | 1617C |

| | | |
|---|---|---|
| 1063 | 747C > T | Silent |
| 1617 | 1301C > T | 3′ |

| | | | | |
|---|---|---|---|---|
| J03037 | J03037 | 259730 | GEN-2I | Carbonic anhydrase II |

| | | |
|---|---|---|
| 627 | 562C > T | Silent |
| 1334 | 1269A > C | 3′ |
| 1487 | 1422A > C | 3′ |

| | | | | |
|---|---|---|---|---|
| M14565 | M14565 | 118485 | GEN-30 | “Cytochrome P450, subfamily XIA |

(cholesterol side chain cleavage)”

| | | |
|---|---|---|
| 947 | 903G > C | M301I |

| M15856 | | M15856 | | 238600 | | GEN-33 | | Lipoprotein lipase |
|---|---|---|---|---|---|---|---|---|
| | 843C | 1553C | 1611G | 2743T | 2851A | 2958G | 3017T | 3272C |
| | 843C | 1553T | 1611G | 2743C | 2851A | 2958G | 3017T | |
| | 843C | 1553T | 1611G | 2743C | 2851A | 2958G | 3017C | |
| | 843C | 1553C | 1611G | 2743C | 2851A | 2958G | 3017T | 3272C |
| | 843C | 1553C | 1611A | 2851A | 2958G | 3017T | | |
| | 843C | 1553C | 1611G | 2743C | 2958G | 3017T | 3272T | |
| | 843T | 1553C | 1611G | 2743C | 2958G | 3017T | | |
| | 843C | 1553C | 1611G | 2743C | 2958A | 3017T | 3272C | |
| | 843C | 1553C | 1611G | 2743C | 2958G | 3017C | | |
| | 1553C | 1611G | 2743C | 2958G | 3017T | 3272T | | |
| | 843C | 1553C | 1611G | 2743C | 2958G | 3017T | 3272C | |
| | 843C | 1553C | 1611A | 2743T | 2958G | 3017T | 3272T | |
| | 843C | 1553T | 1611G | 2743C | 2851A | 2958G | 3017C | 3272T |
| | 843C | 1553C | 1611G | 2743C | 2958A | 3017T | 3272C | |
| | 843C | 1553C | 1611G | 2743C | 2851A | 2958G | 3017C | |
| | 843C | 1553C | 1611A | 2743T | 2851A | 2958G | 3017T | 3272T |
| | 843C | 1553T | 1611G | 2743C | 2851A | 2958G | 3017T | 3272C |

| | | |
|---|---|---|
| 843 | 669C > T | Silent |
| 1553 | 1379C > T | A460V |
| 1611 | 1437G > A | 3′ |
| 1973 | 1799T > C | 3′ |
| 2428 | 2254T > A | 3′ |
| 2743 | 2569T > C | 3′ |
| 2851 | 2677A > G | 3′ |
| 2958 | 2784G > A | 3′ |
| 3017 | 2843T > C | 3′ |
| 3272 | 3098T > C | 3′ |
| 3343 | 3169T > C | 3′ |
| 3447 | 3273C > T | 3′ |

| | | | | |
|---|---|---|---|---|
| M16541 | M16541 | 177400 | GEN-35 | Butyryicholinesterase |

| | | |
|---|---|---|
| 978 | 849G > C | E283D |
| 1828 | 1699G > A | A567T |
| 2127 | 1998A > G | 3′ |

| | | | | |
|---|---|---|---|---|
| M21054 | M21054 | 172410 | GEN-3B | Phospholipase A-2 (PLA-2) lung |

| | | |
|---|---|---|
| 331 | 294G > A | Silent |
| 400 | 363C > A | D121E |

| | | | | |
|---|---|---|---|---|
| M26062 | M26062 | 146710 | GEN-3D | Interleukin 2 receptor beta |
| chain | | | | |

| | | | | | |
|---|---|---|---|---|---|
| 2202C | 2231A | 2287A | 2492T | 2895A | |
| 881C | 2202C | 2231A | 2287G | 2492T | 2895A |
| 881C | 2202G | 2231A | 2287G | 2492T | 2895A |
| 881T | 2202G | 2231A | 2287G | 2492T | 2895A |
| 2202C | 2231A | 2287G | 2492G | 2895A | |
| 881C | 2202C | 2231C | 2287G | | |
| 2202G | 2895G | | | | |
| 881T | 2202C | 2231A | 2287G | 2492T | 2895A |
| 881C | 2202C | 2895G | | | |
| 881T | 2202C | 2231A | 2287A | 2492T | 2895A |
| 881C | 2202C | 2231C | 2287G | 2492T | 2895A |
| 881T | 2202C | 2231A | 2287G | 2492G | 2895A |
| 881T | 2202C | 2895A | | | |
| 881T | 2202G | 2895G | | | |

| | | |
|---|---|---|
| 881 | 750C > T | Silent |
| 2202 | 2071G > C | 3′ |
| 2231 | 2100A > C | 3′ |
| 2287 | 2156G > A | 3′ |
| 2492 | 2361T > G | 3′ |
| 2895 | 2764A > C | 3′ |
| 3158 | 3027A > C | 3′ |

| | | | | |
|---|---|---|---|---|
| M26383 | M26383 | 146930 | GEN-3E | Interleukin 8 |

| | |
|---|---|
| 919C | 1237A |
| 919A | 1237T |
| 919A | 1237A |
| 919C | 1237T |

| | | |
|---|---|---|
| 259 | 185C > G | A62G |
| 919 | 845A > C | 3′ |
| 1237 | 1163A > T | 3′ |
| 1281 | 1207A > G | 3′ |

| | | | | |
|---|---|---|---|---|
| M29696 | M29696 | 146661 | GEN-3H | Interleukin 7 receptor |

| | | | |
|---|---|---|---|
| 154C | 219T | 434G | 1263C |
| 154C | 219T | 434A | 1263C |
| 154C | 434A | 1263T | |
| 154C | 219C | 434A | 1263C |
| 154T | 1263C | | |
| 154C | 219T | 434G | 1263T |
| 154C | 219T | 434A | 1263T |
| 154T | 219T | 434G | 1263C |

| | | |
|---|---|---|
| 154 | 132C > T | Silent |
| 219 | 197C > T | T66I |
| 434 | 412A > G | I138V |
| 1088 | 1066G > A | V356I |
| 1263 | 1241C > T | T414M |

| | | | | |
|---|---|---|---|---|
| M29874 | M29874 | 123930 | GEN-3I | “Cytochrome P450, subfamily IIB |

(phenobarbital-inducible), polypeptide 6”

| | | |
|---|---|---|
| 2758 | 2752T > A | 3′ |
| 2836 | 2830G > A | 3′ |
| 2902 | 2896T > C | 3′ |

| | | | | |
|---|---|---|---|---|
| M34986 | M34986 | 133171 | GEN-3O | Erythropoietin receptor |

| | | |
|---|---|---|
| 1138 | 1138C > G | P380A |

| | | | | |
|---|---|---|---|---|
| M55040 | M55040 | 100740 | GEN-3Q | acetyloholinesterase |

| | | |
|---|---|---|
| 323C | 1213C | 1587C |
| 323T | 1213C1587C | |
| 1213C | 1587T | |
| 1213C | 1587C | |
| 1213A | 1587T | |
| 1213C | 1587T | |

| | | |
|---|---|---|
| 323 | 167C > T | P56L |
| 1154 | 998T > A | V333E |
| 1213 | 1057C > A | H353N |
| 1482 | 1326G > T | Silent |
| 1587 | 1431C > T | Silent |
| 1663 | 1507T > C | F503L |

| | | | | |
|---|---|---|---|---|
| M58525 | M58525 | 116790 | GEN-3S | Catechol-O-methyltransferase |

| | | | | |
|---|---|---|---|---|
| 390T | 418G | 423G | 676A | 813C |
| 676A | 813T | | | |
| 390T | 418G | 423G | 676G | 813C |
| 390C | 418G | 423G | 676G | 813C |
| 390C | 418G | 423A | 676A | 813C |
| 390T | 418G | 423A | 676A | 813C |
| 390C | 418G | 423G | 676A | 813C |
| 390T | 418T | 423G | 676A | 813C |
| 676A | 813T | | | |

| | | |
|---|---|---|
| 390 | 186T > C | Silent |
| 418 | 214G > T | A72S |
| 423 | 2190 > A | Silent |
| 612 | 408C > G | Silent |
| 676 | 472A > G | M158V |
| 813 | 609C > T | Silent |
| 1031 | 827delC | 3′ |
| 1039 | 835C > A | 3′ |

| | | | | |
|---|---|---|---|---|
| M64592 | M64592 | 120420 | GEN-3X | Granulocyte colony-stimulating |
| factor | | | | |

| | | |
|---|---|---|
| 271 | 271T > G | Y91D |
| 1533 | 1533C > T | Silent |

| | | | | |
|---|---|---|---|---|
| M69177 | M69177 | 309860 | GEN-3Y | Monoamine oxidase B |

| | | |
|---|---|---|
| 1685G | 1860A | 1875C |
| 1685G | 1860A | 1875T |
| 1685G | 1860G | 1875T |
| 1685T | 1860A | 1875T |

| | | |
|---|---|---|
| 1685 | 1608G > T | 3′ |
| 1860 | 1783A > G | 3′ |
| 1875 | 1798T > C | 3′ |

| | | | | |
|---|---|---|---|---|
| M69226 | M69226 | 309850 | GEN-3Z | Monoamine oxidase A |

| | | |
|---|---|---|
| 941 | 891T > G | Silent |
| 1373 | 1323G > A | Frame |
| 1460 | 1410C > T | Silent |

| | | | | |
|---|---|---|---|---|
| M80646 | M80646 | 274180 | GEN-40 | Thromboxane synthase |

| | | |
|---|---|---|
| 654A | 943A | 1240C |
| 654C | 943A | 1240G |
| 654C | 943G | 1240C |
| 654C | 943A | 1240C |

| | | |
|---|---|---|
| 654 | 483C > A | D161E |
| 756 | 585G > C | Silent |
| 943 | 772A > G | K258E |
| 1240 | 1069C > G | L357V |

| | | | | |
|---|---|---|---|---|
| M84747 | M84747 | 300007 | GEN-45 | Interleukin 9 receptor |

| | | |
|---|---|---|
| 1273 | 1094G > A | R365H |

| | | | | |
|---|---|---|---|---|
| U00672 | U00672 | 146933 | GEN-4A | Interleukin 10 receptor |

| | | | | |
|---|---|---|---|---|
| 536G | 1033C | 1112A | 1699C | 2148T |
| 536A | 1033T | 1112G | 1699C | 2148T |
| 536A | 1033C | 1112G | 1699C | 2148C |
| 536A | 1033C | 1112G | 1699C | 2148T |
| 536A | 1033C | 1112A | 1699C | 2148T |
| 536A | 1033C | 1112G | 1699T | |
| 536A | 1033C | 1112A | 1699C | |
| 536A | 1033C | 1112G | 1699T | 2148C |
| 1033C | 1112G | 1699C | 2148T | |

| | | |
|---|---|---|
| 536 | 475A > G | 5159G |
| 1033 | 972C > T | Silent |
| 1112 | 1051G > A | G351R |
| 1699 | 1638C > T | Silent |
| 2148 | 2087T > C | 3′ |
| 3377 | 3316A > C | 3′ |
| 3524 | 3463A > G | 3′ |

| | | | | |
|---|---|---|---|---|
| U08092 | U08092 | None | GEN-4C | Histamine N-methyltransferase |

| | | |
|---|---|---|
| 594 | 555G > A | Silent |
| 978 | 939G > A | 3′ |
| 1136 | 1097T > A | 3′ |

| | | | | |
|---|---|---|---|---|
| U19487 | U19487 | 176804 | GEN-4I | “PROSTAGLANDIN E2 RECEPTOR, EP2 |
| SUBTYPE” | | | | |

| | | | | |
|---|---|---|---|---|
| 85G | 231A | 1269C | 1295C | 1442A |
| 85G | 231T | 1269C | 1295C | 1442A |
| 85G | 231A | 1269C | 1295T | 1442G |
| 85A | 231A | 1269C | 1295C | 1442A |
| 85A | 231A | 1269G | 1295C | 1442A |
| 85A | 231A | 1269C | 1295T | 1442G |
| 85G | 231A | 1269G | 1295T | 1442G |
| 85G | 231A | 1269C | 1295T | 1442A |
| 85G | 231T | 1269C | 1295T | 1442G |
| 85G | 231A | 1269C | 1295C | 1442G |
| 85A | 231A | 1269C | 1295T | 1442A |
| 85G | 231A | 1269G | 1295C | 1442G |
| 231A | 1269C | 1295T | 1442G | |
| 85G | 231T | 1269G | 1295T | 1442G |
| 85A | 231A | | | |

| | | |
|---|---|---|
| 85 | (−72)A > G | 5′ |
| 231 | 75A > T | Silent |
| 1269 | 1113C > G | 3′ |
| 1295 | 1139C > T | 3′ |
| 1442 | 1286A > G | 3′ |

| | | | | |
|---|---|---|---|---|
| U31628 | U31628 | 601070 | GEN-4J | Interleukin 15 receptor alpha |
| chain | | | | |

| | |
|---|---|
| 627A | 892G |
| 627C | 892G |
| 627G | 892A |
| 627A | 892A |

| | | |
|---|---|---|
| 627 | 545A > C | N182T |
| 892 | 810G > A | 3′ |
| 1250 | 1168G > T | 3′ |

| | | | | |
|---|---|---|---|---|
| U70136 | U70136 | 600044 | GEN-4R | Thrombopoletin |

| | | |
|---|---|---|
| 4138 | 4105G > T | A1369S |
| 4141 | 4108T > A | F13701 |

| U70867 | | U70867 | | 601460 | | GEN-4S | | prostaglandin transporter hPGT | | | | |
|---|---|---|---|---|---|---|---|---|---|---|---|---|
| | 301A | 931A | 1069A | 1888C | 2014C | 2323A | 2706T | 2839T | 2908A | 3171G | 3253A | 3255G |
| | 3594T | | | | | | | | | | | |
| | 301G | 931A | 1069A | 1888C | 2706T | 2839A | 2908A | 3171G | 3253A | 3594T | | |
| | 301G | 931A | 1069A | 1888C | 2014C | 2323A | 2706T | 2839T | 2908A | 3171G | 3253A | 3594A |
| | 301G | 931A | 1069A | 1888C | 2014C | 2323A | 2706T | 2839T | 2908A | 3171G | 3253G | 3594T |
| | 301A | 931A | 1069A | 1888C | 2014C | 2323A | 2706T | 2839T | 2908A | 3171G | 3253A | 3255A |
| | 3594T | | | | | | | | | | | |
| | 301A | 931G | 1069A | 1888C | 2014C | 2706T | 2839A | 3171G | 3255A | | | |
| | 301G | 931A | 1069A | 1888C | 2014C | 2323A | 2706T | 2839T | 2908A | 3171G | 3253A | 3255G |
| | 3594T | | | | | | | | | | | |
| | 301G | 931A | 1069A | 1888C | 2014C | 2323A | 2706T | 2839T | 2908A | 3171G | 3253A | 3255A |
| | 3594T | | | | | | | | | | | |
| | 301G | 931A | 1069A | 1888C | 2323T | 2706T | 2839T | 2908A | 3171G | 3253G | 3594T | |
| | 301G | 931A | 1069A | 1888T | 2014C | 2323A | 2706T | 2839T | 2908A | 3253A | 3255A | 3594T |
| | 301G | 1069A | 1888C | 2014C | 2323A | 2706T | 2908G | 3253A | 3255A | 3594T | | |
| | 931G | 1069A | 1888T | 2014C | 2323A | 2706T | 2839T | 2908A | 3171G | 3253A | 3255A | 3594T |
| | 301G | 931A | 1069A | 1888C | 2323T | 2706T | 2839T | 2908A | 3253A | 3255A | 3594T | |
| | 301G | 1069G | 1888C | 2014C | 2323A | 2706T | 2839T | 2908A | 3171G | 3255G | 3594T | |
| | 301A | 931A | 1069A | 1888C | 2014C | 2323A | 2706T | 2839T | 2908A | 3171G | 3253G | 3255G |
| | 3594T | | | | | | | | | | | |
| | 931A | 1069G | 1888C | 2014C | 2323A | 2839T | 2908A | 3171G | 3253A | 3255A | 3594T | |
| | 301G | 931G | 1069A | 1888C | 2014C | 2323A | 2706T | 2839T | 2908A | 3171G | 3253A | 3255A |
| | 3594T | | | | | | | | | | | |
| | 301G | 931G | 1069A | 1888C | 2706T | 2839A | 2908A | 3171G | 3253A | 3594T | | |
| | 301G | 931A | 1069A | 1888C | 2014C | 2323A | 2706T | 2839T | 2908A | 3171A | 3253A | 3255A |
| | 3594T | | | | | | | | | | | |
| | 301A | 931G | 1069A | 1888C | 2014C | 2323A | 2706T | 2839T | 2908A | 3171G | 3253A | 3255A |
| | 3594T | | | | | | | | | | | |
| | 301A | 931G | 1069A | 1888C | 2014C | 2323A | 2706T | 2839T | 2908A | 3171A | 3253A | 3594T |
| | 301G | 931A | 1069A | 1888C | 2014C | 2323A | 2706T | 2839A | 2908G | 3171G | 3253A | 3255A |
| | 3594T | | | | | | | | | | | |
| | 301G | 931A | 1069A | 1888C | 2014C | 2323T | 2706T | 2839A | 2908A | 3171G | 3253A | 3594T |
| | 301G | 931G | 1069A | 1888C | 2014T | 2323A | 2706T | 2839A | 2908A | 3171G | 3253A | 3594T |
| | 301G | 931A | 1069A | 1888C | 2014C | 2323T | 2706T | 2839T | 2908A | 3171G | 3253G | 3255A |
| | 3594T | | | | | | | | | | | |
| | 931A | 1069G | 1888C | 2014C | 2323A | 2706G | 2839T | 2908A | 3171G | 3253A | 3255A | 3594T |
| | 931A | 1069A | 1888C | 2014C | 2323A | 2706T | 2839T | 2908A | 3171A | 3253G | 3255A | 3594T |
| | 2014C | 2323A | 2706T | 2839T | 2908A | 3171G | 3253G | 3255A | 3594T | | | |
| | 301G | 931A | 1069A | 1888C | 2014C | 2323A | 2706T | 2839T | 2908A | 3171G | 3253A | 3255G |
| | 3594A | | | | | | | | | | | |
| | 301A | 931A | 1069A | 1888C | 2014C | 2323A | 2706T | 2839T | 2908A | 3171G | 3253G | 3255A |
| | 3594T | | | | | | | | | | | |
| | 301A | 931G | 1069A | 1888C | 2014C | 2323A | 2706T | 2839A | 3171G | 3255A | | |
| | 301G | 931G | 1069A | 1888C | 2014C | 2323T | 2706T | 2839A | 3171G | 3255A | | |
| | 2706T | 2839T | 2908A | 3594T | | | | | | | | |
| | 301G | 931A | 1069G | 1888C | 2014C | 2323A | 2706T | 2839T | 2908A | 3171G | 3253G | 3255G |
| | 3594T | | | | | | | | | | | |
| | 301G | 931A | 1069A | 1888C | 2014C | 2323T | 2706T | 2839T | 2908A | 3171G | 3253A | 3255A |
| | 3594T | | | | | | | | | | | |
| | 301G | 1069A | 1888C | 2014C | 2323A | 2706T | 2839T | 2908A | 3171G | 3253A | 3255A | 3594T |

| | | |
|---|---|---|
| 301 | 210G > A | Silent |
| 931 | 840A > G | Silent |
| 1069 | 978k > G | Silent |
| 1888 | 1797C > T | Silent |
| 2014 | 1923C > T | Silent |
| 2323 | 2232k > T | 3′ |
| 2706 | 2615T > G | 3′ |
| 2839 | 2748T > A | 3′ |
| 2908 | 2817k > G | 3′ |
| 3171 | 3080k > G | 3′ |
| 3253 | 3162k > G | 3′ |
| 3255 | 3164k > G | 3′ |
| 3594 | 3503T > k | 3′ |

| | | | | |
|---|---|---|---|---|
| X03663 | X03663 | 164770 | GEN-51 | Colony stimulating factor 1 |
| receptor | | | | |

| | | | | | | |
|---|---|---|---|---|---|---|
| 1026C | 1135G | 1385A | 2835C | 3254T | 3255C | 3530C |
| 1026C | 1135G | 1385A | 2835C | 3254T | 3255C | 3530A |
| 1026T | 1135G | 1385A | 2835C | 3254T | 3255A | 3530C |
| 1026T | 1135G | 1385A | 2835G | 3254T | 3530C | |
| 1026C | 1135G | 1385A | 2835G | 3254T | 3530C | |
| 1026C | 1135G | 1385A | 2835C | 3254C | 3255A | 3530C |
| 1026T | 1135G | 1385A | 2835C | 3254C | 3255A | 3530C |
| 1026T | 1135G | 1385A | 2835C | 3254T | 3255C | 3530C |
| 1135G | 1385G | 2835C | 3254T | 3255A | 3530C | |
| 1026C | 1135G | 1385G | 2835C | 3254T | 3255C | 3530C |
| 1026C | 1135A | 2835C | 3254T | 3255C | | |
| 1026C | 1135G | 1385A | 2835C | 3254T | 3255A | 3530C |
| 1026C | 1135A | 1385G | 2835C | 3254T | 3255C | 3530C |
| 1026C | 1135G | 1385A | 2835G | 3254T | 3255A | 3530C |
| 1026C | 1135A | 1385G | 2835C | 3254C | 3255A | 3530C |
| 1026T | 1135G | 1385A | 2835G | 3254T | 3255C | 3530C |
| 1026T | 1135G | 1385G | 2835C | 3254T | 3255A | 3530C |
| 1026T | 1135G | 1385G | 2835C | 3254T | 3255C | 3530C |
| 1026T | 1135A | 1385G | 2835C | 3254C | 3530C | |
| 1026C | 1135G | 1385G | 2835C | 3254C | 3255A | 3530C |

| | | |
|---|---|---|
| 1026 | 726T > C | Silent |
| 1135 | 835G > A | V279M |
| 1385 | 1085A > G | H362R |
| 2835 | 2535C > G | Silent |
| 3254 | 2954T > C | 3′ |
| 3255 | 2955C > A | 3′ |
| 3530 | 3230C > A | 3′ |
| 3732 | 3432T > C | 3′ |
| 3951 | 3651C > A | 3′ |

| | | | | |
|---|---|---|---|---|
| X03884 | X03884 | 186830 | GEN-52 | “CD3E antigen, epsilon |

polypeptide (TiT3 complex)”

| | | |
|---|---|---|
| 108 | 54C > T | Silent |
| 726 | 672C > A | 3′ |
| 1258 | 1204T > A | 3′ |

| | | | | |
|---|---|---|---|---|
| X13589 | X13589 | 107910 | GEN-56 | Aromatase (CYPl9) , cDNA |

| | |
|---|---|
| 625C | 914T |
| 625C | 914C |
| 625A | 914T |

| | | |
|---|---|---|
| 364 | 240A > G | Silent |
| 625 | 501C > A | Silent |
| 914 | 790C > T | R264C |
| 1655 | 1531C > T | 3′ |
| 1796 | 1672G > T | 3′ |

| | | | | |
|---|---|---|---|---|
| X52425 | X52425 | 147781 | GEN-59 | Interleukin 4 receptor |

| | | |
|---|---|---|
| 170 | (−6)C > G | 5′ |
| 398 | 223A > G | I75V |
| 412 | 237C > T | Silent |
| 676 | 501C > T | Silent |
| 943 | 768C > G | Silent |
| 1114 | 939T > C | Silent |
| 1211 | 1036A > G | I346V |
| 1374 | 1199A > C | E400A |
| 1417 | 1242G > T | Silent |
| 1474 | 1299T > C | Silent |
| 1682 | 1507T > C | S503P |
| 1730 | 1555C > T | Silent |
| 1902 | 1727A > G | Q576R |
| 2198 | 2023C > T | P675S |
| 2572 | 2397T > C | Silent |
| 2659 | 2484T > C | 3′ |
| 2661 | 2486T > C | 3′ |
| 2741 | 2566C > G | 3′ |
| 2892 | 2717G > A | 3′ |
| 3044 | 2869G > A | 3′ |
| 3289 | 3114A > G | 3′ |
| 3391 | 3216C > T | 3′ |
| 3419 | 3244G > C | 3′ |

| | | | | |
|---|---|---|---|---|
| X83861 | X83861 | 176806 | GEN-5H | Prostaglandin E receptor 3 |

(subtype EP3) {alternative products}

| | |
|---|---|
| 801G | 825T |
| 801A | 825G |
| 801G | 825G |

| | | |
|---|---|---|
| 387 | 180C > G | Silent |
| 801 | 594G > A | Silent |
| 825 | 618G > T | Silent |

| | | | | |
|---|---|---|---|---|
| K03001 | K03001 | 100650 | GEN-5N | Aldehyde dehydrogenase 2, |

mitochondrial

| | | |
|---|---|---|
| 656 | 656T > A | V219E |
| 988 | 988G > C | V330L |
| 1156 | 1156G > A | E386K |

| | | | | |
|---|---|---|---|---|
| L78207 | L78207 | 600509 | GEN-5Q | Cell surface receptor for |

sulfonylureas on pancreatic b cells

| | | |
|---|---|---|
| 4019 | 3981A > G | Silent |

| | | | | |
|---|---|---|---|---|
| M11220 | M11220 | 138960 | GEN-5R | Granulocyte colony-stimulating |
| factor | | | | |

| | |
|---|---|
| 82C | 382T |
| 82T | 382C |
| 82C | 382C |

| | | |
|---|---|---|
| 82 | 50C > T | S17F |
| 382 | 350T > C | I117T |

| | | | | |
|---|---|---|---|---|
| M59941 | M59941 | 138981 | GEN-62 | −Granulocyte-macrophage (Colony |

stimulating factor 2 receptor, beta, low-affinity)”

| | | | |
|---|---|---|---|
| 773C | 847C | 855T | 881G |
| 773G | 847G | 855T | 881G |
| 773C | 847G | 855T | 881G |
| 773G | 855T | 881A | |
| 773G | 847C | 855T | 881G |
| 773G | 847C | 855C | 881G |
| 773G | 847G | 855T | 881A |

| | | |
|---|---|---|
| 773 | 745G > C | E249Q |
| 847 | 819C > G | Silent |
| 855 | 827T > C | L276P |
| 881 | 853G > A | G285R |

| | | | | |
|---|---|---|---|---|
| M73832 | M73832 | 425000 | GEN-63 | GRANULOCYTE-MACROPHAGE COLONY- |

STIMULATING FACTOR RECEPTOR ALPHA CHAIN PRECURSOR

| | | |
|---|---|---|
| 488 | 470C > G | Frame |

| | | | | |
|---|---|---|---|---|
| M74782 | M74782 | 308385 | GEN-64 | “Interleukin 3 receptor, alpha |

(low affininty)”

| | | |
|---|---|---|
| 952 | 806C > T | T269I |
| 1396 | 1250C > T | 3′ |

| | | | | |
|---|---|---|---|---|
| M96652 | M96652 | 147851 | GEN-65 | Interleukin 5 receptor alpha |

| | |
|---|---|
| 101G | 145T |
| 101A | 145T |
| 101G | 145C |

| | | |
|---|---|---|
| 101 | (−149)G > A | 5′ |
| 145 | (−105)T > C | 5′ |
| 883 | 634T > G | S212A |

| U73338 | | U73338 | | 156570 | | GEN-69 | | Methionine Synthase | | | | |
|---|---|---|---|---|---|---|---|---|---|---|---|---|
| | 194C | 284C | 1136G | 1252C | 1334G | 1699T | 3207G | 3209G | 3885G | 5444C | 5551G | 5573C |
| | 5659T | 5678T | 5934G | | | | | | | | | |
| | 194C | 1136G | 1252C | 1334G | 1699T | 3207G | 3209C | 5444C | 5551G | 5659T | 5678T | 5934A |
| | 194C | 284C | 1136A | 1252C | 1334G | 1699T | 3207G | 3209G | 3538A | 3885G | 3886C | 5573C |
| | 5659T | 5678T | 5934A | | | | | | | | | |
| | 194C | 284C | 1136G | 1334G | 1699T | 3150A | 3207T | 3209G | 5444C | 5551G | 5573C | 5659T |
| | 5678T | 5874C | 5934A | | | | | | | | | |
| | 194C | 284C | 1136G | 1252C | 1334G | 1699T | 3150A | 3207G | 3209G | 5444C | 5551G | 5573T |
| | 5678T | 5874C | 5934A | | | | | | | | | |
| | 194C | 284C | 1136G | 1252C | 1334G | 1699T | 3150A | 3207G | 3209G | 3538G | 3885G | 3886A |
| | 5444C | 5551G | 5573C | 5659T | 5678T | 5874C | 5934A | | | | | |
| | 194C | 284C | 1136G | 1252C | 1334G | 1699T | 3150G | 3207G | 3209G | 3538A | 3885G | 3886C |
| | 5444C | 5551G | 5573C | 5659T | 5678T | 5874T | 5934A | | | | | |
| | 194C | 284C | 1136G | 1252C | 1334G | 1699C | 3207G | 3209G | 5444C | 5551G | 5573C | 5659T |
| | 5678T | 5934A | | | | | | | | | | |
| | 194C | 284C | 1136G | 1252C | 1334G | 1699T | 3150A | 3207G | 3209G | 3538A | 3885G | 3886C |
| | 5444A | 5551A | 5573C | 5659T | 5678T | 5874C | 5934A | | | | | |
| | 194C | 284C | 1136G | 1252C | 1334G | 1699T | 3150A | 3207G | 3209G | 3538A | 3886C | 5444C |
| | 5551G | 5573C | 5678C | 5874C | 5934A | | | | | | | |
| | 194C | 284C | 1136G | 1252C | 1334G | 1699T | 3150A | 3207G | 3209G | 3885G | 3886A | 5444A |
| | 5573C | 5659T | 5678T | 5874C | 5934A | | | | | | | |
| | 194C | 284C | 1136G | 1252C | 1334G | 1699T | 3150A | 3207G | 3209G | 3538A | 3886C | 5444C |
| | 5551G | 5573C | 5678T | 5874C | 5934A | | | | | | | |
| | 194C | 284C | 1136G | 1252C | 1334G | 1699T | 3150A | 3207G | 3209G | 3538A | 3885G | 3886C |
| | 5444A | 5551A | 5573C | 5659T | 5678T | 5874C | 5934A | | | | | |
| | 194C | 284C | 1136G | 1252T | 1334G | 1699T | 3150A | 3207G | 3209G | 3538G | 3885G | 3886A |
| | 5444C | 5551G | 5S73C | 5659T | 5678T | 5874C | 5934A | | | | | |
| | 194C | 284C | 1136G | 1252C | 1699T | 3150A | 3207G | 3209G | 3538G | 3885G | 5444C | 5551A |
| | 5573C | 5659T | 5678T | 5874C | 5934A | | | | | | | |
| | 194C | 284C | 1136G | 1252C | 1334G | 1699T | 3150G | 3207G | 3209G | 3885G | 5444C | 5551G |
| | 5573C | 5659T | 5678T | 5874C | 5934A | | | | | | | |
| | 194C | 284C | 1136G | 1252C | 1334G | 1699T | 3150A | 3207G | 3209G | 5444C | 5551A | 5573C |
| | 5659T | 5678T | 5874C | 5934A | | | | | | | | |
| | 194C | 284C | 1136G | 1252C | 1334G | 1699T | 3150G | 3207G | 3209G | 3538G | 3885G | 3886C |
| | 5444C | 5551G | 5573C | 5659T | 5678T | 5874C | 5934A | | | | | |
| | 194C | 284C | 1136G | 1334G | 1699T | 3150A | 3207T | 3209G | 5444C | 5551G | 5573C | 5659T |
| | 5678T | 5874C | 5934A | | | | | | | | | |
| | 194C | 284C | 1136G | 1252C | 1334G | 1699T | 3538A | 3885G | 3886C | 5444A | 5551A | 5573T |
| | 5659T | 5678T | 5874C | 5934A | | | | | | | | |
| | 194C | 284C | 1136G | 1252C | 1334G | 1699T | 3150G | 3207G | 3209G | 5444C | 5551G | 5573C |
| | 5659T | 5678T | 5874T | 5934A | | | | | | | | |
| | 194C | 284C | 1136G | 1252C | 1334G | 1699T | 3150A | 3207G | 3209G | 3538A | 3885G | 3886C |
| | 5444C | 5551G | 5573C | 5659C | 5678C | 5874C | 5934A | | | | | |
| | 194C | 284C | 1136G | 1252C | 1334G | 1699C | 3150G | 3207G | 3209G | 5444C | 5551G | 5573C |
| | 5659T | 5678T | 5874T | 5934A | | | | | | | | |
| | 194C | 284C | 1136G | 1252C | 1334G | 1699T | 3150G | 3207G | 3209G | 3538A | 3885G | 3886C |
| | 5444A | 5551A | 5573C | 5659T | 5678T | 5874T | 5934A | | | | | |
| | 194C | 284C | 1136G | 1252C | 1334G | 1699T | 3150A | 3207G | 3209G | 3538G | 3885G | 3886A |
| | 5444A | 5551A | 5573C | 5659T | 5678T | 5874C | 5934A | | | | | |
| | 194C | 284C | 1136C | 1252C | 1334G | 1699T | 3150G | 3207G | 3209G | 5659C | 5678C | 5874T |
| | 5934A | | | | | | | | | | | |
| | 194C | 284C | 1136G | 1252C | 1334G | 1699T | 3150A | 3207G | 3209G | 5444C | 5551G | 5573C |
| | 5659T | 5678T | 5874C | 5934A | | | | | | | | |
| | 194C | 284C | 1136G | 1252C | 1334G | 1699T | 3150G | 3207G | 3209G | 3538G | 3885G | 3886C |
| | 5444C | 5551G | 5573G | 5659T | 5678T | 5874T | 5934A | | | | | |
| | 194C | 284C | 1136G | 1252C | 1334G | 1699T | 3207G | 3209G | 5444A | 5551A | 5573C | 5659C |
| | 5678T | 5874C | 5934A | | | | | | | | | |
| | 194C | 284C | 1136G | 1252C | 1334G | 1699T | 3150A | 3207G | 3209G | 5444A | 5551A | 5573C |
| | 5659T | 5678T | 5874C | 5934A | | | | | | | | |
| | 194C | 284C | 113GA | 1252C | 1334G | 1699T | 3150G | 3207G | 3209G | 3538A | 3885G | 3886C |
| | 5444A | 5551A | 5573C | 5659T | 5678T | 5874T | 5934A | | | | | |
| | 194C | 284C | 1136G | 1252C | 1334G | 1699T | 3150G | 3207G | 3209G | 3538G | 3885G | 3886C |
| | 5444C | 5551G | 5573C | 5659T | 5678T | 5874T | 5934G | | | | | |
| | 194C | 284T | 1136G | 252C | 1334G | 1699T | 3150G | 3207G | 3209C | 5444C | 5551G | 5573T |
| | 5659T | 5678T | 5874T | 5934A | | | | | | | | |
| | 194C | 284C | 113GG | 1252C | 1334A | 1699T | 3150A | 3207G | 3209G | 3538G | 3885G | 3886C |
| | 5444C | 5551A | 5573C | 5659T | 5678T | 5874C | 5934A | | | | | |
| | 194C | 284C | 1136G | 1252C | 1334G | 1699T | 3150A | 3207G | 3209G | 5444C | 5551G | 5573T |
| | 5659C | 5678T | 5874C | 5934A | | | | | | | | |

| | | |
|---|---|---|
| 194 | (−201)C > G | 5′ |
| 284 | (−111)C > T | 5′ |
| 1136 | 742G > A | V248M |
| 1158 | 764G > A | C255Y |
| 1252 | 858C > T | Silent |
| 1334 | 940G > A | D314N |
| 1699 | 1305T > C | Silent |
| 3150 | 2756A > G | D919G |
| 3207 | 2813G > T | S938I |
| 3209 | 2815G > C | G939R |
| 3538 | 3144G > A | Silent |
| 3885 | 3491G > A | R1164H |
| 3886 | 3492C > A | Silent |
| 5095 | 4701G > A | 3′ |
| 5444 | 5050C > A | 3′ |
| 5551 | 5157G > A | 3′ |
| 5573 | 5179C > T | 3′ |
| 5659 | 5265T > C | 3′ |
| 5678 | 5284T > C | 3′ |
| 5874 | 5480C > T | 3′ |
| 5934 | 5540A > G | 3′ |
| 6750 | 6356G > A | 3′ |

| | | | | |
|---|---|---|---|---|
| X01057 | X01057 | 147730 | GEN-6B | Interleukin-2 receptor (IL-2R) |

| | | |
|---|---|---|
| 264G | 696C | 891G |
| 264A | 696C | 891A |
| 264G | 696T | 891A |
| 264G | 696C | 891A |

| | | |
|---|---|---|
| 264 | 84G > A | Silent |
| 696 | 516C > T | Silent |
| 891 | 711A > G | Silent |

| | | | | |
|---|---|---|---|---|
| M29882 | M29882 | 107670 | GEN-6R | Apolipoprotein A-II |

| | | |
|---|---|---|
| 26 | 17C > A | AGE |
| 183 | 174G > A | Silent |
| 192 | 183C > A | Silent |

| | | | | |
|---|---|---|---|---|
| X07282 | X07282 | 180220 | GEN-72 | RETINOIC ACID RECEPTOR BETA-2 |

| | |
|---|---|
| 1532G | 1664A |
| 1532A | 16640 |
| 1532G | 1664G |

| | | |
|---|---|---|
| 1532 | 1189G > A | G397R |
| 1664 | 1321G > A | V4411 |

| | | | | |
|---|---|---|---|---|
| X52773 | X52773 | 180245 | GEN-74 | Retinoid X receptor, alpha |

| | |
|---|---|
| 140T | 363A |
| 140C | 363A |
| 140C | 363G |

| | | |
|---|---|---|
| 140 | 65C > T | P22L |
| 363 | 288A > G | Silent |
| 1744 | 1669G > A | 3′ |

| | | | | |
|---|---|---|---|---|
| X63522 | X63522 | 180246 | GEN-75 | Retinoic X receptor beta, |

partial cDNA

| | | |
|---|---|---|
| 1331 | 1152T > C | Silent |

| | | | | |
|---|---|---|---|---|
| D25418 | D25418 | 600022 | GEN-78 | Prostaglandin I2 (prostacyclin) |

receptor(IP)

| | | | | | |
|---|---|---|---|---|---|
| 250C | 1075A1 | 562C | | | |
| 250C | 1075A | 1562G | | | |
| 1047G | 1075C | 1332C | | | |
| 250G | 1047C | 1075A | 1332C | 1562C | |
| 250G | 1047C | 1075C | 1332C | 1562C | |
| 250G | 1047C | 1075C | 1332C | 1562G | |
| 250C | 726G | 1047C | 1075C | 1332C | 1562C |
| 250G | 1047C | 1332T | 1562C | | |
| 250G | 1075A | 1332C | 1562G | | |
| 250C | 1075A | 1562C | | | |
| 250C | 1047G | 1075C | 1332C | 1562G | |
| 250C | 1075A | 1562G | | | |
| 250C | 726A | 1047C | 1075C | 1332C | 1562C |
| 250G | 1047C | 1075C | 1332C | 1562G | |
| 250G | 1047C | 1075C | 1332C | 1562C | |
| 250G | 1075A | 1332C | 1562C | | |
| 250G | 1047C | 1075C | 1332T | | |
| 250C | 1047C | 1075C | 1332C | | |
| 250C | 1047C | 1075C | 1332C | 1562C | |
| 250G | 1047C | 1075C | 1332T | 1562C | |
| 250C | 1047C | 1075C | 1332C | 1562G | |
| 250G | 1075A | 1332C | 1562G | | |

| | | |
|---|---|---|
| 250 | 159G > C | Silent |
| 726 | 635G > A | R212H |
| 1047 | 956C > G | S319W |
| 1075 | 984A > C | Silent |
| 1332 | 1241C > T | 3′ |
| 1562 | 1471C > G | 3′ |

| | | | | |
|---|---|---|---|---|
| PTGER2 | L28175 | 601586 | GEN-7C | Prostaglandin E receptor 2 |

(subtype EP2), 53kD

| | | |
|---|---|---|
| 547C | 1268G | 1725G |
| 547T | 1268G | 1725A |
| 547C | 1268A | 1725A |
| 547C | 1268G | 1725A |
| 547C | 1268G | |

| | | |
|---|---|---|
| 547 | 159C > T | Silent |
| 611 | 223G > A | V75M |
| 1268 | 880G > A | V294I |
| 1725 | 1337A > G | Q446R |

| | | | | |
|---|---|---|---|---|
| M16505 | M16505 | 308100 | GEN-7D | STERYL-SULFATASE PRECURSOR |

| | | |
|---|---|---|
| 2725 | 2505T > G | 3′ |
| 4364 | 4144G > A | 3′ |
| 4665 | 4445A > G | 3′ |
| 5894 | 5674A > G | 3′ |

| | | | | |
|---|---|---|---|---|
| M68874 | M68874 | 600522 | GEN-7K | Cytosolic phospholipase A2, cDNA |

| | | |
|---|---|---|
| 2743 | 2605G > A | 3′ |

| | | | | |
|---|---|---|---|---|
| U07132 | U07132 | 600380 | GEN-7M | Orphan receptor |

| | | |
|---|---|---|
| 763 | 519G > A | Silent |
| 1399 | 1155C > T | Silent |
| 1726 | 1482G > C | 3′ |
| 1952 | 1708C > G | 3′ |

| | | | | |
|---|---|---|---|---|
| X77307 | X77307 | 601122 | GEN-7T | Serotonin 5-HT receptors 5-HT2B |

| | | | | | | |
|---|---|---|---|---|---|---|
| 99C | 207G | 677G | 783C | 894A | 1307T | 1369C |
| 99C | 207G | 677G | 783T | 894A | 1307C | 1369C |
| 99C | 207G | 677T | 783C | 894A | 1307C | 1369C |
| 99C | 207A | 677G | 783C | 1307C | | |
| 99T | 207G | 677G | 783C | 894A | | |
| 99C | 207G | 677G | 783C | 894A | 1307C | 1369C |
| 207A | 783C | 894C | | | | |
| 207G | 783C | 894A | 1307C | 1369C | | |
| 99C | 207A | 677G | 783C | 894C | 1307C | 1369T |

| | | |
|---|---|---|
| 99 | 44C > T | P15L |
| 207 | 152G > A | G51E |
| 677 | 622G > T | V208L |
| 783 | 728C > T | A243V |
| 894 | 839A > C | K280T |
| 1307 | 1252C > T | R418W |
| 1369 | 1314C > T | Silent |

| | | | | |
|---|---|---|---|---|
| X57830 | X57830 | 182135 | GEN-7V | Serotonin receptor 5HT-2A, cDNA |

| | | |
|---|---|---|
| 1384 | 1239A > T | Silent |
| 1499 | 1354C > T | H452Y |
| 1962 | 1817A > C | 3′ |

| | | | | |
|---|---|---|---|---|
| M11050 | M11050 | 138040 | GEN-7Y | Glucocorticoid receptor |

| | |
|---|---|
| 1220A | 1896C |
| 1220A | 1896T |
| 1220G | 1896C |

| | | |
|---|---|---|
| 1220 | 1088A > G | N363S |
| 1896 | 1764C > T | Silent |
| 2166 | 2034C > T | Silent |
| 3353 | 3221T > G | 3′ |
| 3398 | 3266T > G | 3′ |

| | | | | |
|---|---|---|---|---|
| M24857 | M24857 | 180190 | GEN-80 | Retinoic acid receptor, gamma |

| | | |
|---|---|---|
| 1694 | 1280C > T | S427L |

| | | | | |
|---|---|---|---|---|
| U25029 | U25029 | 138040 | GEN-82 | Glucocorticoid receptor alpha |

| | | |
|---|---|---|
| 335C | 386T | 1069C |
| 335C | 386C | 1069C |
| 335C | 386T | 1069T |
| 335T | 386T | 1069C |
| 335C | 386T | |

| | | |
|---|---|---|
| 335 | 335C > T | 3′ |
| 386 | 386T > C | 3′ |
| 1069 | 1069C > T | 3′ |

| | | | | |
|---|---|---|---|---|
| J03817 | J03817 | 138350 | GEN-9D | Glutathione S-transferase M1 |

| | | |
|---|---|---|
| 99 | 84T > C | Silent |
| 543 | 528C > T | Silent |
| 643 | 628T > A | S210T |
| 728 | 713C > G | 3′ |
| 902 | 887C > T | 3′ |

| | | | | |
|---|---|---|---|---|
| K03191 | K03191 | 108330 | GEN-9E | Cytochrome 9450, subfamily I |

(aromatic compound-inducible), polypeptide 1

| | | |
|---|---|---|
| 1470 | 1384G > A | V462I |

| | | | | |
|---|---|---|---|---|
| M63012 | M63012 | 168820 | GEN-9F | Paraoxonase 1 |

| | | |
|---|---|---|
| 172 | 163A > T | M55L |

| | | | | |
|---|---|---|---|---|
| M63509 | M63509 | 138380 | GEN-9G | Glutathione S-transferase M2 |
| (muscle) | | | | |

| | | |
|---|---|---|
| 644 | 628A > T | T210S |

| | | | | |
|---|---|---|---|---|
| M64082 | M64082 | 136130 | GEN-9H | Flavin-containing monooxygenas |

1 (DIMETHYLANILINE MONOOXYGENASE)

| | | | |
|---|---|---|---|
| 1808C | 1818G | 1830G | 1904T |
| 1808T | 1818G | 1830G | 1904C |
| 1808C | 1818G | 1830A | |
| 1808T | 1818G | 1830G | 1904T |
| 1808C | 1818A | 1904T | |
| 1808C | 1818G | 1830G | 1904C |
| 1808C | 1818A | 1830A | 1904T |
| 1808T | 1818A | 1830A | 1904T |
| 1808C | 1818G | 1830A | 1904T |

| | | |
|---|---|---|
| 1286 | 1188A > G | Silent |
| 1808 | 1710C > T | 3′ |
| 1818 | 1720G > A | 3′ |
| 1830 | 1732G > A | 3′ |
| 1904 | 1806C > T | 3′ |

| | | | | |
|---|---|---|---|---|
| M96234 | M96234 | 138333 | GEN-9J | Glutathione S-transferase M4 |

| | | |
|---|---|---|
| 797 | 534T > C | Silent |

| | | | | |
|---|---|---|---|---|
| X03674 | X03674 | 305900 | GEN-9K | Glucose-6-phosphate |

dehydrogenase

| | |
|---|---|
| 672G | 1438T |
| 672A | 1438T |
| 672G | 1438C |

| | | |
|---|---|---|
| 503 | 33C > G | H11Q |
| 589 | 119C > T | S40L |
| 672 | 202G > A | V68M |
| 846 | 376A > G | N126D |
| 1438 | 968T > C | L323P |
| 2215 | 1745T > C | 3′ |
| 2242 | 1772T > C | 3′ |
| 2341 | 1871G > A | 3′ |

| | | | | |
|---|---|---|---|---|
| Y00498 | Y00498 | 601129 | GEN-9N | Cytochrome P450, subfamily IIC |

(mephenytoin 4-hydroxylase)

| | |
|---|---|
| 678G | 723A |
| 678G | 723G |
| 678A | 723A |

| | | |
|---|---|---|
| 431 | 389C > A | T130N |
| 489 | 447T > C | Silent |
| 491 | 449A > G | H150R |
| 522 | 480G > T | K160N |
| 525 | 483T > C | Silent |
| 582 | 540C > T | Silent |
| 583 | 541G > A | V181I |
| 678 | 636G > A | Frame |
| 723 | 681A > G | Silent |
| 834 | 792C > G | I264M |
| 999 | 957C > G | Silent |
| 1539 | 1497T > C | 3′ |

| | | | | |
|---|---|---|---|---|
| AB000410 | AB000410 | 601982 | GEN-9O | Human hOGG1 mRNA, complete cds |

| | |
|---|---|
| 251G | 682T |
| 251T | 682C |
| 251G | 682C |

| | | |
|---|---|---|
| 251 | (−18)G > T | 5′ |
| 682 | 414C > T | Silent |

| | | | | |
|---|---|---|---|---|
| AF001437 | AF001437 | 245349 | GEN-9T | Dihydrolipoamide S- |

acetyltransferase (E2 component of pyruvate dehydrogenase complex)

| | | |
|---|---|---|
| 75 | 67T > C | C23R |
| 116 | 108C > T | Silent |
| 759 | 751T > G | S251A |
| 806 | 798C > T | Silent |
| 866 | 858T > C | Silent |
| 2000 | 1992G > T | 3′ |
| 2158 | 2150C > A | 3′ |

| | | | | |
|---|---|---|---|---|
| D13811 | D13811 | 238310 | GEN-AA | Glycine cleavage system: Protein |
| T | | | | |

| | | | |
|---|---|---|---|
| 277G | 1073A | 1083G | 1773C |
| 277T | 1073G | 1083G | 1773C |
| 277G | 1073G | 1083G | 1773C |
| 277G | 1073G | 1083G | 1773T |

| | | |
|---|---|---|
| 277 | 148G > T | V50L |
| 1073 | 944G > A | R315K |
| 1083 | 954G > A | Silent |
| 1773 | 1644C > T | 3′ |
| 2037 | 1908C > T | 3′ |

| | | | | |
|---|---|---|---|---|
| J03490 | J03490 | 246900 | GEN-C5 | Dihydrolipoamide dehydrogenase |

(E3 component of pyruvate dehydrogenase complex, 2-oxo-glutarate complex,
branched chain keto acid dehydrogenase complex)

| | | | | |
|---|---|---|---|---|
| 1427C | 1624A | 1634T | 1813A | 2096T |
| 1427T | 1624T | 1634G | 1813A | 2096T |
| 1427C | 1624A | 1634G | 1813A | 2096C |
| 1427C | 1624T | 1634T | 1813A | 2096T |
| 1427C | 1624T | 1634G | 1813A | 2096T |
| 1427C | 1624A | 1634G | 1813A | 2096T |
| 1427C | 1624A | 1634G | 1813G | |
| 1427T | 1624A | l634G | 1813A | 2096T |
| 1624A | 1634G | 1813G | 2096C | |
| 1427C | 1624T | 1634G | 1813A | 2096C |
| 1427C | 1624A | 1634G | 1813G | 2096C |
| 1427C | 1624T | 1634G | 1813G | 2096C |
| 1427C | 1624A | 1634G | 2096T | |

| | | |
|---|---|---|
| 1427 | 1351C > T | Silent |
| 1569 | 1493A > C | N498T |
| 1624 | 1548T > A | 3′ |
| 1634 | 1558G > T | 3′ |
| 1813 | 1737A > G | 3′ |
| 2096 | 2020T > C | 3′ |

| | | | | |
|---|---|---|---|---|
| J04031 | J04031 | 172460 | GEN-CB | Methenyltetrahydrofolate |

cyclohydrolase

| | | | | | | |
|---|---|---|---|---|---|---|
| 454G | 969C | 1614C | 2011A | 2358C | 2368G | 2486C |
| 454G | 969C | 1614C | 2011G | 2358C | 2368G | 2486C |
| 454G | 969C | 1614C | 2011A | 2358T | 2368G | 2486C |
| 454A | 969C | 1614C | 2011A | 2358C | 2368G | 2486T |
| 454A | 969C | 1614C | 2011G | 2358C | 2368G | 2486C |
| 454G | 969C | 1614T | 2011A | 2358C | 2368G | 2486C |
| 454A | 969C | 1614C | 2011A | 2358T | 2368G | 2486C |
| 454G | 969C | 1614C | 2011A | 2358C | 2368G | 2486T |
| 454G | 969C | 1614C | 2011G | 2358C | 2486C | |
| 454A | 969C | 1614C | 2011G | 2358C | 2368G | 2486T |
| 454A | 969C | 1614C | 2011A | 2358C | 2368G | 2486C |
| 454G | 969C | 1614C | 2011G | 2358C | 2368G | 2486T |
| 454A | 969C | 1614C | 2011G | | | |
| 454G | 969C | 1614C | 2011G | 2358C | 2368G | 2486C |
| 454A | 969C | 1614C | 2011A | | | |
| 454G | 969C | 1614C | 2011A | | | |
| 454G | 969C | 1614C | 2011G | | | |

| | | |
|---|---|---|
| 454 | 401G > A | R134K |
| 969 | 916C > G | Q306E |
| 1614 | 1561T > C | Silent |
| 2011 | 1958G > A | R653Q |
| 2335 | 2282C > T | T761M |
| 2358 | 2305C > T | L769F |
| 2368 | 2315G > A | R772H |
| 2486 | 2433C > T | Silent |

| | | | | |
|---|---|---|---|---|
| L11696 | L11696 | 104614 | GEN-D6 | Solute carrier family 3 |

(cystine, dibasic and neutral amino acid transporters, activator of cystine,
dibasic and neutral amino acid transport), member 1

| | | |
|---|---|---|
| 1897 | 1854G > A | M618I |
| 2232 | 2189T > C | 3′ |

| | | | | |
|---|---|---|---|---|
| L14754 | L14754 | 600502 | GEN-D9 | DNA-binding protein (SMBP2) |

| | | |
|---|---|---|
| 2129 | 2080C > T | R694W |
| 2365 | 2316C > T | Silent |
| 3696 | 3647C > T | 3′ |
| 3712 | 3663T > C | 3′ |
| 3771 | 3722C > G | 3′ |

| | | | | |
|---|---|---|---|---|
| L19067 | L19067 | 164014 | GEN-DE | TRANSCRIPTION FACTOR P65 |

| | | | |
|---|---|---|---|
| 1130G | 1708A | 1936G | 2024C |
| 1130A | 1708A | 1936C | |
| 1130G | 1708A | 1936C | 2024C |
| 1130G | 1708G | 1936C | 2024C |
| 1936C | 2024C | | |
| 1130A | 1708A | 1936C | 2024T |

| | | |
|---|---|---|
| 1129 | 1091C > T | S364L |
| 1130 | 1092G > A | Silent |
| 1708 | 1670A > G | 3′ |
| 1936 | 1898G > C | 3′ |
| 2024 | 1986C > T | 3′ |

| | | | | |
|---|---|---|---|---|
| L20298 | L20298 | 121360 | GEN-DH | Transcription Factor (CBFB) |

| | | |
|---|---|---|
| 2696 | 2696A > G | 3′ |

| | | | | |
|---|---|---|---|---|
| L31801 | L31801 | 600682 | GEN-DQ | Solute carrier family 16 |

(monocarboxylic acid transporters) , member 1

| | | | |
|---|---|---|---|
| 1021G | 1416A | 1482A | 1660T |
| 1021A | 1416A | 1482A | 1660T |
| 1021G | 1416A | 1482T | 1660G |
| 1021G | 1416G | 1660T | |
| 1021G | 1416A | 1482T | 1660T |
| 1021G | 1416G | 1482A | 1660T |

| | | |
|---|---|---|
| 1021 | 1009G > A | V337I |
| 1416 | 1404A > G | I468M |
| 1482 | 1470A > T | E490D |
| 1660 | 1648T > G | 3′ |
| 1772 | 1760G > C | 3′ |

| | | | | |
|---|---|---|---|---|
| M16827 | M16827 | 201450 | GEN-EI | Acyl-Coenzyme A dehydrogenase, |

C-4to C-12 straight chain

| | |
|---|---|
| 918C | 1179G |

| | | |
|---|---|---|
| 918 | 900C > T | Silent |
| 1179 | 1161A > G | Silent |
| 1956 | 1938T > C | 3′ |

| | | | | |
|---|---|---|---|---|
| M26393 | M26393 | 201470 | GEN-EW | Acyl-Coenzyme A dehydrogenase, |

C-2 to C-3 short chain

| | | | | |
|---|---|---|---|---|
| 353T | 657G | 1022T | | |
| 353C | 657G | 1022C | 1386A | |
| 353C | 657A | 1022C | 1386A | |
| 657A | 1022C | 1292G | 1386G | |
| 353C | 657G | 1022C | 1292G | 1386G |
| 353C | 657G | 1022T | 1292G | 1386G |
| 1022C | 1292C | 1386G | | |
| 353T | 657G | 1022C | 1292G | 1386G |
| 353C | 657A | | | |
| 353C | 657G | | | |
| 353T | 657G | 1022T | 1292G | 1386G |
| 353T | 1022C | 1292C | 1386G | |
| 353T | 657G | 1022T | | |
| 353T | 657A | 1022T | 1292G | 1386G |
| 353T | 657A | 1022C | | |
| 353T | 657A | | | |
| 353T | 657A | 1022C | 1292C | 1386G |
| 353T | 657G | | | |
| 353T | 657A | 1022C | 1292G | 1386G |

| | | |
|---|---|---|
| 353 | 321T > C | Silent |
| 657 | 625G > A | G209S |
| 1022 | 990C > T | Silent |
| 1292 | 1260G > C | 3′ |
| 1386 | 1354G > A | 3′ |
| 1797 | 1765A > G | 3′ |

| | | | | |
|---|---|---|---|---|
| M30938 | M30938 | 194364 | GEN-F5 | ATP-DEPENDENT DNA HELICASE II, |

86 KD SUBUNIT

| | | | |
|---|---|---|---|
| 1599G | 2549T | 2953A | |
| 1599G | 2549T | 2953C | |
| 1599A | 2549T | 2953A | 3067A |
| 1599A | 2549T | 2953C | 3067A |
| 1599A | 2549C | 2953A | 3067A |
| 1599G | 2549C | 2953A | |
| 1599A | 2549T | 2953C | 3067G |
| 1599A | 2549C | 2953A | 3067G |
| 1599A | 2549T | 2953A | |
| 1599G | 2549T | 2953A | 3067G |
| 1599A | 2549T | 2953C | |

| | | |
|---|---|---|
| 1599 | 1572A > G | Silent |
| 2549 | 2522T > C | 3′ |
| 2953 | 2926C > A | 3′ |
| 3037 | 3010G > A | 3′ |
| 3067 | 3040G > A | 3′ |

| | | | | |
|---|---|---|---|---|
| M31523 | M31523 | 147141 | GEN-F7 | Transcription factor 3 (E2A |

immunoglobulin enhancer binding factors E12/E47)

| | | |
|---|---|---|
| 1321 | 1291G > A | G431S |
| 1323 | 1293C > T | Silent |
| 1332 | 1302G > A | Silent |
| 1338 | 1308T > C | Silent |
| 1608 | 1578C > G | Silent |
| 4022 | 3992G > A | 3′ |
| 4254 | 4224T > A | 3′ |

| | | | | |
|---|---|---|---|---|
| M34479 | M34479 | 179060 | GEN-F9 | Pyruvate dehydrogenase |

(lipoamide) beta

| | | |
|---|---|---|
| 109 | 109G > A | D37N |
| 438 | 438A > G | Silent |
| 1172 | 1172A > C | 3′ |
| 1179 | 1179C > T | 3′ |
| 1323 | 1323C > A | 3′ |
| 1376 | 1376G > C | 3′ |
| 1433 | 1433C > T | 3′ |

| | | | | |
|---|---|---|---|---|
| M55531 | M55531 | 138230 | GEN-FF | Solute carrier family 2 |

(facilitated glucose transporter) , member 5

| | | |
|---|---|---|
| 1208 | 1133T > G | V378G |
| 1975 | 1900C > T | 3′ |
| 1985 | 1910A > G | 3′ |

| | | | | |
|---|---|---|---|---|
| M60761 | M60761 | 156569 | GEN-FL | O-6-methylguanine-DNA |

methyltransferase

| | | |
|---|---|---|
| 174T | 265C | 442A |
| 174C | 265T | 442A |
| 174C | 265C | 442A |
| 174T | 265T | 442A |
| 174C | 265C | 442G |
| 174T | 265T | |
| 174T | 265T | 442G |

| | | |
|---|---|---|
| 174 | 159C > T | Silent |
| 264 | 249A > T | Silent |
| 265 | 250C > T | L84F |
| 442 | 427A > G | I143V |

| | | | | |
|---|---|---|---|---|
| M81181 | M81181 | 182331 | GEN-G4 | ATPase, Na+/K+ transporting, |

beta 2 polypeptide

| | | |
|---|---|---|
| 107 | (−301)C > G | 5′ |
| 1070 | 663C > A | Silent |
| 1745 | 1338A > G | 3′ |
| 1845 | 1438C > G | 3′ |
| 1891 | 1484G > A | 3′ |
| 1974 | 1567C > A | 3′ |
| 2364 | 1957T > C | 3′ |

| | | | | |
|---|---|---|---|---|
| M81768 | M81768 | 107310 | GEN-G6 | Solute carrier family 9 |

(sodium/hydrogen exchanger)

| | | |
|---|---|---|
| 3042 | 2989G > A | 3′ |

| | | | | |
|---|---|---|---|---|
| M84739 | M84739 | 109091 | GEN-GB | CALRETICULTN PRECURSOR |

| | | |
|---|---|---|
| 1416 | 1308T > G | 3′ |
| 1695 | 1587G > A | 3′ |

| | | | | |
|---|---|---|---|---|
| M94859 | M94859 | 114217 | GEN-GP | Calnexin |

| | | |
|---|---|---|
| 79 | (−17)C > T | 5′ |
| 2678 | 2583C > T | 3′ |
| 3011 | 2916G > T | 3′ |
| 3527 | 3432T > G | 3′ |

| U09178 | | U09178 | | 274270 | | GEN-HA | | Dihydropyrimidine Dehydrogenase | | | | |
|---|---|---|---|---|---|---|---|---|---|---|---|---|
| | 166T | 577A | 635A | 1452C | 1557G | 1575A | 1708A | 1977T | 3432T | 3652C | 3730G | 3925G |
| | 3937C | | | | | | | | | | | |
| | 166T | 577A | 638A | 1452C | 1557A | 1575A | 1708A | 1977T | 3432T | 3682C | 3730G | 3925G |
| | 3937C | | | | | | | | | | | |
| | 166T | 577G | 638A | 1452C | 1557G | 1575A | 1708A | 1977T | 3432T | 3682C | 3730G | 3937C |
| | 166T | 577A | 638A | 1452C | 1557G | 1575A | 1708A | 1977T | 3432T | 3682C | 3730G | 3925A |
| | 3937C | | | | | | | | | | | |
| | 166T | 577A | 638A | 1452C | 1557G | 1575A | 1708A | 1977T | 3432C | 3682C | 3730G | 3937T |
| | 166C | 577A | 638A | 1452C | 1557G | 1575A | 1705A | 1977T | 3432T | 3682C | 3730G | 3925G |
| | 3937C | | | | | | | | | | | |
| | 166C | 577A | 635A | 1452C | 1557G | 1575A | 1708A | 1977T | 3432T | 3682C | 3730G | 3925A |
| | 3937T | | | | | | | | | | | |
| | 166T | 577A | 638A | 1452C | 1557G | 1575A | 1708A | 1977T | 3432C | 3682C | 3730G | 3937C |
| | 166T | 577A | 638A | 1452C | 1557G | 1575A | 1708A | 1977T | 3432T | 3682C | 3730G | 3925A |
| | 3937T | | | | | | | | | | | |
| | 577A | 638A | 1452C | 1557G | 1575A | 1977T | 3432T | 3652T | 3730G | | | |
| | 166C | 577A | 638A | 1452C | 1557G | 1575A | 1708A | 1977C | 3432T | 3682C | 3730G | |
| | 577A | 1452C | 1557G | 1575G | 1708A | 1977T | 3432T | 3682C | 3730G | 3925G | 3937C | |
| | 166T | 577A | 638A | 1452C | 1557G | 1575A | 1708G | 1977T | 3432T | 3682C | 3730G | 3925A |
| | 3937C | | | | | | | | | | | |
| | 638A | 1452C | 1557G | 1575A | 1708A | 1977T | 3432T | 3682C | 3730A | 3925A | 3937T | |
| | 166C | 577A | 638A | 1557G | 1575A | 1708A | 1977T | 3432T | 3682C | 3730G | 3925A | 3937C |
| | 166T | 577A | 638A | 1452C | 1557G | 1575A | 1708A | 1977T | 3432T | 3682C | 3730G | |
| | 166T | 577A | 638A | 1452C | 1557A | 1575A | 1708G | 1977T | 3432C | 3682C | 3730G | |
| | 166C | 577G | | | | | | | | | | |
| | 166T | 577A | 638A | 1452C | 1557G | 1575A | 1708A | 1977T | 3432T | 3682T | 3730G | 3925A |
| | 3937C | | | | | | | | | | | |
| | 166T | 3925A | 3937T | | | | | | | | | |
| | 166T | 577G | 3925G | 3937C | | | | | | | | |
| | 166T | 577A | 1452C | 1557G | 1575A | 1708G | 1977T | 3682C | 3730G | 3925A | 3937C | |
| | 166T | 577A | 1452C | 1557G | 1575A | 1708A | 1977T | 3432T | 3682C | 3730G | 3925A | 3937C |
| | 166T | 577G | | | | | | | | | | |
| | 166T | 577A | 638A | 1452C | 1557G | 1575G | 1708A | 1977T | 3432T | 3682C | 3730G | 3925G |
| | 3937C | | | | | | | | | | | |
| | 166T | 577A | 3925A | | | | | | | | | |
| | 166T | 577A | 638A | 1452C | 1557G | 1575A | 1708G | 1977T | 3432T | 3682C | 3730A | |
| | 166T | 577A | 3925A | 3937T | | | | | | | | |
| | 166C | 577A | | | | | | | | | | |
| | 166T | 577A | 638A | 1452C | 1557G | 1575A | 1708A | 1977T | 3432C | 3682C | 3730G | 3925A |
| | 3937T | | | | | | | | | | | |
| | 166T | 577A | 3925G | 3937C | | | | | | | | |
| | 166T | 577A | 3925A | 3937C | | | | | | | | |
| | 166T | 577A | 638A | 1452C | 1557G | 1575A | 1708G | 1977T | 3432T | 3682C | 3730G | |
| | 166C | 577A | 638A | 1452T | 1557G | 1575A | 1708A | 1977T | 3432T | 3682C | 3720G | 3925A |
| | 3937T | | | | | | | | | | | |
| | 166T | 577G | 638A | 1452C | 1557G | 1575A | 1708A | 1977T | 3432T | 3682C | 3730G | 3925A |
| | 3937C | | | | | | | | | | | |
| | 166T | 577A | 3937G | | | | | | | | | |
| | 166T | 577G | 638A | 1452C | 1557G | 1575A | 1708A | 1977T | 3432T | 3682C | 3730G | 3925A |
| | 3937T | | | | | | | | | | | |

| | | |
|---|---|---|
| 166 | 85T > C | C29R |
| 577 | 496A > G | M166V |
| 638 | 557A > G | Y186C |
| 1452 | 1371C > T | Silent |
| 1557 | 1476G > A | Silent |
| 1575 | 1494A > G | Silent |
| 1708 | 1627A > G | I543V |
| 1977 | 1896C > T | Silent |
| 3432 | 3351T > C | 3′ |
| 3682 | 3601C > T | 3′ |
| 3730 | 3649G > A | 3′ |
| 3925 | 3844A > G | 3′ |
| 3937 | 3856T > C | 3′ |

| U19720 | | U19720 | | 600424 | | GEN-I1 | | Folate Transporter (SLC19A1) | |
|---|---|---|---|---|---|---|---|---|---|
| | 175A | 341C | 1067A | 1337C | 1997T | 2652T | | | |
| | 175G | 341C | 791T | 1067G | 1337C | 1997T | 2582G | 2617T | 2652T |
| | 175A | 341C | 79lT | 1067G | 1337C | 1997C | 2582G | 26l7T | 2652T |
| | 175A | 341C | 791T | 1067G | 1337C | 1997T | 2617C | 2652T | |
| | 175G | 341C | 791C | 1067A | 1337C | 1997T | 2582T | 2617C | 2652T |
| | 175A | 341C | 791T | 1067G | 1337C | 1997T | 2582G | 2617T | 2652T |
| | 175G | 341C | 791C | 1067G | 1337C | 1997T | 2582T | 2617C | 2652T |
| | 341C | 791C | 1067G | 1337G | 1997T | 2582G | 2617T | 2652T | |
| | 175A | 341C | 791C | 1067G | 1997T | 2582T | 2617C | | |
| | 175G | 341C | 791T | 1067G | 1337C | 1997T | 2582G | 2652T | |
| | 175A | 341C | 791T | 1067G | 1337C | 1997T | 2582T | 2617T | 2652T |
| | 175A | 341C | 791T | 1067G | 1337C | 1997T | 2582G | 2652T | |
| | 175G | 341C | 791T | 1067G | 1337C | 1997T | 2582T | 2617T | 2652T |
| | 175A | 341C | 791T | 1337A | 1997T | 2582G | 2617T | 2652C | |
| | 175G | 341C | 791C | 1067G | 1337A | 1997T | 2582T | 2617C | 2652T |
| | 175G | 341C | 791C | 1067G | 1337C | 1997T | 2582G | 2617T | 2652T |
| | 175A | 341C | 791C | 1337C | 1997T | 2582G | 2617T | 2652T | |
| | 175A | 341C | 791C | 1067A | 1337C | 1997T | 2582T | 2617C | 2652T |
| | 175G | 341C | 791C | 1067G | 1337C | 1997T | 2582T | 2617T | 2652T |
| | 175A | 34lC | 791T | 1067G | 1337C | 1997T | 2582T | 2617C | 2652T |

| | | |
|---|---|---|
| 53 | (−43)T > C | 5′ |
| 175 | 80G > A | R27H |
| 341 | 246C > G | Silent |
| 791 | 696C > T | Silent |
| 1067 | 972G > A | Silent |
| 1337 | 1242C > A | Silent |
| 1997 | 1902T > C | 3′ |
| 2100 | 2005\hat{ }2006insG | 3′ |
| 2582 | 2487T > G | 3′ |
| 2617 | 2522C > T | 3′ |
| 2652 | 2557T > C | 3′ |

| | | | | |
|---|---|---|---|---|
| U36601 | U36601 | 603268 | GEN-IR | Heparan N-deacetylase/N- |

sulfotransferase-2

| | | |
|---|---|---|
| 2727 | 2700T > G | 3′ |
| 2972 | 2945A > G | 3′ |

| | | | | |
|---|---|---|---|---|
| U45730 | U48730 | 601511 | GEN-JA | Transcription Factor Stat5b |

| | | |
|---|---|---|
| 494 | 484T > C | Silent |
| 496 | 486A > G | Silent |
| 499 | 489A > G | Silent |
| 502 | 492G > A | Silent |
| 570 | 560G > C | G187A |
| 573 | 563C > A | P188Q |
| 1003 | 993G > A | Silent |
| 1063 | 1053T > C | Silent |
| 1066 | 1056G > A | Silent |
| 1105 | 1095C > T | Silent |
| 1159 | 1149C > T | Silent |
| 1969 | 1959C > T | Silent |

| | | | | |
|---|---|---|---|---|
| X02317 | X02317 | 147450 | GEN-KM | Superoxide dismutase 1 (Cu/Zn) |

| | | | |
|---|---|---|---|
| 614 | 550A > C | tr,26 3′ | +TL,30 |

| | | | | |
|---|---|---|---|---|
| X03747 | X03747 | 182330 | GEN-KR | ATFase, Na+/K+ transporting, |

beta 1 polypeptide

| | | |
|---|---|---|
| 447 | 321G > A | Silent |
| 1516 | 1390G > T | 3′ |
| 2182 | 2056C > T | 3′ |

| | | | | |
|---|---|---|---|---|
| X13403 | X13403 | 164175 | GEN-L8 | POU domain, class 2, |

transcription factor 1

| | | |
|---|---|---|
| 1298 | 1239T > C | Silent |
| 1476 | 1417G > A | A473T |

| | | | | |
|---|---|---|---|---|
| X16396 | X16396 | None | GEN-LC | Methenyltetrahydrofolate |

dehydrogenase

| | | | | | |
|---|---|---|---|---|---|
| 608A | 1259G | 1284C | 1392T | 1397A | 1480G |
| 608A | 1259G | 1284T | 1392T | 1397A | 1480G |
| 1397G | 1480A | | | | |
| 608A | 1259G | 1284C | 1392T | 1397A | 1480A |
| 608A | 1259G | 1284T | 1392T | 1397A | 1480A |
| 608A | 1259A | 1284C | 1392T | 1397A | 1480A |
| 608A | 1259G | 1284C | 1392C | 1397A | 1480A |
| 608G | 1259G | 1397A | 1480G | | |
| 1397G | 1480A | | | | |
| 1397A | 1480A | | | | |
| 1397A | 1480G | | | | |

| | | |
|---|---|---|
| 608 | 593A > G | N198S |
| 1259 | 1244G > A | 3′ |
| 1284 | 1269C > T | 3′ |
| 1392 | 1377T > C | 3′ |
| 1397 | 1382A > G | 3′ |
| 1480 | 1465G > A | 3′ |

| | | | | |
|---|---|---|---|---|
| X54199 | X54199 | 138440 | GEN-LS | Phosphoribosylglycinamide |

formyltransferase, phosphoribosylglycinamide synthetase,
phosphoribosylaminoimidazole synthetase

| | | | |
|---|---|---|---|
| 1339A | 1999G | 2333A | |
| 1339A | 1999C | 2333G | 2682T |
| 1339A | 1999C | 2333A | 2682T |
| 1339G | 1999C | 2333A | 2682T |
| 1339A | 1999G | 2333A | 2682T |
| 1339A | 1999C | 2333A | |
| 1339A | 1999G | 2333A | 2682C |

| | | |
|---|---|---|
| 168 | 90G > A | Silent |
| 1339 | 1261G > A | V421I |
| 1999 | 1921C > G | P641A |
| 2333 | 2255A > G | D752G |
| 2682 | 2604T > C | Silent |

| | | | | |
|---|---|---|---|---|
| V00594 | V00594 | 156360 | GEN-P6 | Human mRNA for metallothionein |

from cadmium-treated cells

| | | |
|---|---|---|
| 320 | 263G > C | 3′ |

| | | | | |
|---|---|---|---|---|
| K01171 | K01171 | 142860 | GEN-PB | Human HLA-DR alpha-chain mRNA |

| | | |
|---|---|---|
| 297 | 283T > C | Silent |
| 416 | 402C > A | Silent |
| 665 | 651C > T | Silent |
| 738 | 724G > T | V242L |
| 748 | 734G > A | S245N |
| 797 | 783A > G | 3′ |
| 842 | 828A > G | 3′ |
| 901 | 887G > A | 3′ |
| 928 | 914T > A | 3′ |
| 933 | 919T > A | 3′ |
| 942 | 928C > T | 3′ |
| 954 | 940G > A | 3′ |
| 999 | 985T > G | 3′ |
| 1035 | 1021A > C | 3′ |
| 1077 | 1063C > T | 3′ |
| 1091 | 1077C > G | 3′ |
| 1154 | 1140A > C | 3′ |
| 1171 | 1157T > A | 3′ |

| | | | | |
|---|---|---|---|---|
| X02920 | X02920 | 107400 | GEN-PH | Human mRNA for alpha 1- |

antitrypsin carboxyterminal region (aa 268-394)

| | | |
|---|---|---|
| 107 | 107T > C | L36P |
| 137 | 137G > A | S46N |
| 195 | 195C > T | Silent |
| 327 | 327A > C | E109D |

| | | | | |
|---|---|---|---|---|
| X03069 | X03069 | 142857 | GEN-PI | Human mRNA for HLA-D class II |

antigen DR1 beta chain

| | | |
|---|---|---|
| 99 | 37A > G | T13A |
| 104 | 42G > T | Silent |
| 348 | 286C > A | L96I |
| 361 | 299G > A | R100K |
| 452 | 390G > A | Silent |
| 459 | 397T > G | 5133A |
| 463 | 401A > G | K134R |
| 471 | 409C > A | P137T |
| 500 | 438C > T | Silent |
| 508 | 446G > A | S149N |
| 523 | 461G > C | G154A |
| 547 | 485G > T | R162L |
| 551 | 489C > T | Silent |
| 552 | 490G > A | G164S |
| 567 | 505G > A | A169T |
| 573 | 511G > A | V171M |
| 584 | 522A > G | Silent |
| 593 | 531C > T | Silent |
| 596 | 534G > C | Q178H |
| 605 | 543T > C | Silent |
| 632 | 570G > A | Silent |
| 647 | 585G > A | Silent |
| 686 | 624T > C | Silent |
| 691 | 629C > T | T210M |
| 692 | 630G > A | Silent |
| 716 | 654A > T | R218S |
| 721 | 659G > A | R220Q |
| 756 | 694G > A | V232I |
| 767 | 705C > T | Silent |
| 800 | 738G > A | Silent |
| 814 | 752G > A | R251K |
| 847 | 785C > G | T262R |
| 865 | 803A > G | 3′ |
| 868 | 806C > A | 3′ |
| 893 | 831C > T | 3′ |
| 899 | 837T > C | 3′ |
| 903 | 841A > G | 3′ |
| 913 | 851A > G | 3′ |
| 988 | 926G > A | 3′ |
| 1004 | 942G > A | 3′ |
| 1027 | 965C > T | 3′ |
| 1105 | 1043G > C | 3′ |
| 1128 | 1066C > T | 3′ |
| 1139 | 1077C > T | 3′ |
| 1140 | 1078G > C | 3′ |

| | | | | |
|---|---|---|---|---|
| X03348 | X03348 | 138040 | GEN-PL | Human mRNA for beta- |

glucocorticoid receptor (clone OB10)

| | | |
|---|---|---|
| 3297 | 3165G > A | 3′ |

| | | | | |
|---|---|---|---|---|
| X03438 | X03438 | 138970 | GEN-PM | Human mRNA for granulocyte |

colony-stimulating factor (G-CSF)

| | | |
|---|---|---|
| 586 | 555G > A | Silent |
| 1235 | 1204C > T | 3′ |

| | | | | |
|---|---|---|---|---|
| J04794 | J04794 | 103830 | GEN-PR | Human aldehyde reductase mLRNA, |

complete cds

| | | |
|---|---|---|
| 661 | 601C > A | Q201K |

| | | | | |
|---|---|---|---|---|
| J05176 | J05176 | 107280 | GEN-PT | Human alpha-1-antichymotrypsin |

mRNA, 3′ end

| | |
|---|---|
| 240A | 327C |
| 240A | 327T |
| 240G | 327C |

| | | |
|---|---|---|
| 240 | 240A > G | Silent |
| 327 | 327C > T | Silent |
| 554 | 554T > C | V185A |

| | | | | |
|---|---|---|---|---|
| U07989 | U07989 | 147200 | GEN-Q2 | Human Burkitt′s lymphoma |

immunoglobulin kappa light chain mRNA, partial cds

| | | |
|---|---|---|
| 39 | 39T > A | Silent |
| 307 | 307G > C | V103L |
| 312 | 312A > G | Silent |
| 568 | 568G > C | V190L |
| 610 | 610G > A | V204I |

| | | | | |
|---|---|---|---|---|
| D126l4 | D126l4 | 153440 | GEN-QD | Human mRNA for lymphotoxin (TNF- |

beta), complete cds

| | | |
|---|---|---|
| 319 | 179C > A | T60N |

| | | | | |
|---|---|---|---|---|
| M12807 | M12807 | 186940 | GEN-QG | Human T-cell surface |

glycoprotein T4 mRNA, complete cds

| | | | | | |
|---|---|---|---|---|---|
| 867C | 868C | 1098C | 1416C | 1443C | 1526T |
| 867A | 868C | 1098C | 1416T | 1443C | 1526T |
| 867A | 868C | 1098T | 1416C | 1443C | 1526A |
| 868C | 1098C | 1416C | 1443T | 1526T | |
| 867C | 868C | 1098T | 1416T | 1443C | 1526T |
| 867A | 868C | 1098C | 1416C | 1443C | 1526T |
| 867A | 868C | 1098T | 1416T | 1443C | 1526T |
| 867A | 868T | 1098C | 1416C | 1443C | 1526T |
| 867A | 868C | 1098C | 1416C | 1443C | 1526A |
| 868C | 1098T | 1416T | 1443C | 1526A | |
| 867C | 868C | 1098C | 1416T | 1443C | 1526T |
| 867C | 868C | 1098C | 1416C | 1443T | 1526T |
| 867A | 868T | 1098C | | | |
| 867A | 868T | 1098T | 1416T | 1443C | 1526T |

| | | |
|---|---|---|
| 867 | 792A > C | K264N |
| 868 | 793C > T | R265W |
| 1098 | 1023C > T | Silent |
| 1416 | 1341C > T | Silent |
| 1443 | 1368C > T | Silent |
| 1526 | 1451T > A | 3′ |

| | | | | |
|---|---|---|---|---|
| M12824 | M12824 | 186910 | GEN-QH | Human 1-cell differentiation |

antigen Leu-2/T8 mRNA, partial cds

| | | |
|---|---|---|
| 1545 | 1458C > T | 3′ |
| 1765 | 1678C > T | 3′ |

| | | | | |
|---|---|---|---|---|
| X15722 | X15722 | 138300 | GEN-QR | Human mRNA for glutathione |

reductase (EC 1.6.4.2)

| | | | |
|---|---|---|---|
| 432T | 1044A | | |
| 164T | 432C | 1044A | |
| 164C | 236T | 432C | 1044A |
| 164C | 236C | 432C | 1044A |
| 164T | 236C | 432C | 1044G |
| 164T | 236C | 432C | 1044A |
| 164T | 236C | 432T | 1044A |
| 164C | 236C | 432C | |

| | | |
|---|---|---|
| 164 | 60C > T | Silent |
| 236 | 132T > C | Silent |
| 432 | 328C > T | R110C |
| 1044 | 940A > G | I314V |

| | | | | |
|---|---|---|---|---|
| M15872 | M15872 | 138360 | GEN-QS | Human glutathione S-transferase |

2 (GST) mRNA, complete cds

| | | |
|---|---|---|
| 16 | (−40)G > A | 5′ |
| 54 | (−2)T > C | 5′ |
| 84 | 29T > C | F10S |
| 111 | 56C > T | T19I |
| 170 | 115G > T | Frame |
| 321 | 266G > A | R89K |
| 376 | 321C > T | Silent |
| 430 | 375G > A | Silent |
| 622 | 567C > T | Silent |
| 684 | 629A > C | E210A |
| 701 | 646G > T | A216S |

| | | | | |
|---|---|---|---|---|
| M24400 | M24400 | 118890 | GEN-R2 | Human chymotrypsinogen mRNA, |

complete cds

| | | |
|---|---|---|
| 121 | 105G > A | Silent |
| 231 | 215C > A | T72N |
| 460 | 444C > T | Silent |
| 649 | 633C > T | Silent |

| | | | | |
|---|---|---|---|---|
| M24895 | M24895 | 104660 | GEN-R3 | Homo sapiens alpha-amylase mRNA, |

complete cds

| | | |
|---|---|---|
| 193 | 147C > G | Silent |
| 967 | 921A > G | Silent |
| 1009 | 963G > C | Silent |
| 1027 | 981T > A | Silent |
| 1054 | 1008T > C | Silent |
| 1093 | 1047T > A | Silent |
| 1178 | 1132A > G | N378D |
| 1191 | 1145T > C | I382T |
| 1394 | 1348A > T | T4505 |
| 1474 | 1428T > C | Silent |
| 1492 | 1446C > T | Silent |
| 1504 | 1458C > T | Silent |
| 1543 | 1497G > A | Silent |
| 1579 | 1533A > G | Silent |
| 1601 | 1555T > A | 3′ |

| | | | | |
|---|---|---|---|---|
| M33491 | M33491 | 191080 | GEN-RD | Human tryptase-I mRNA, 3′ end |

| | | | |
|---|---|---|---|
| 92 | 92C > T | +TL,22 | S31L |
| 392 | 392C > G | | T131R |
| 609 | 609G > A | | Silent |
| 707 | 707G > A | | C236Y |
| 730 | 730G > A | | A244T |
| 837 | 837T > G | | 3′ |
| 840 | 840G > T | | 3′ |
| 1008 | 1008T > C | | 3′ |
| 1050 | 1050C > T | | 3′ |
| 1060 | 1060A > G | | 3′ |

| | | | | |
|---|---|---|---|---|
| M55643 | M55643 | 164011 | GEN-RP | Human factor KBF1 mRNA, complete |
| cds | | | | |

| | | |
|---|---|---|
| 1231C | 1324C | 1917A |
| 1231C | 1324T | 1917G |
| 1231C | 1324C | 1917G |
| 1231T | 1324C | 1917G |

| | | |
|---|---|---|
| 1231 | 1050C > T | Silent |
| 1324 | 1143C > T | Silent |
| 1917 | 1736G > A | R579K |
| 1936 | 1755G > A | Silent |

| | | | | |
|---|---|---|---|---|
| X59498 | X59498 | 176300 | GEN-RU | H. sapiens ttr mRNA for |

transthyretin

| | | |
|---|---|---|
| 92 | 71G > A | G24D |
| 97 | 76G > A | G26S |
| 177 | 156G > T | Silent |
| 187 | 166G > A | A56T |
| 292 | 271G > A | V91M |
| 380 | 359C > T | S120F |

| | | | | |
|---|---|---|---|---|
| X62468 | X62468 | 147570 | GEN-RW | H. sapiens mRNA for IFN-gamma |
| (pKC-0) | | | | |

| | | |
|---|---|---|
| 395 | 383G > A | G128E |

| | | | | |
|---|---|---|---|---|
| M68867 | M68867 | 180231 | GEN-S1 | Human cellular retinoic acid- |

binding protein II (CRABP) mRNA, complete cds

| | | |
|---|---|---|
| 604 | 506C > A | 3′ |

| | | | | |
|---|---|---|---|---|
| J05096 | J05096 | 182340 | GEN-SL | alpha-subunit of Na+/K+ ATPase |

isoform2

| | | |
|---|---|---|
| 2364 | 2260T > G | S754A |
| 5295 | 5191G > A | 3′ |

| | | | | |
|---|---|---|---|---|
| PTPRC | Y00062 | 151460 | GEN-SY | Human mRNA for T200 leukocyte |

common antigen (CD45, LC-A)

| | | |
|---|---|---|
| 3437 | 3291T > C | Silent |
| 3441 | 3295G > A | V1099I |

| | | | | |
|---|---|---|---|---|
| HLA-DQA1 | X00033 | 146880 | GEN-TO | Human RNA sequence of the human |

DS glycoprotein alpha subunit from the HLA-D region of the major
histocompatibility complex (MHC)

| | | |
|---|---|---|
| 41 | 22A > C | M8L |
| 79 | 60T > C | Silent |
| 145 | 126C > T | Silent |
| 157 | 138C > G | Silent |
| 162 | 143T > A | F48Y |
| 165 | 146C > G | T495 |
| 227 | 208A > C | K70Q |
| 243 | 224A > G | H75R |
| 248 | 229C > T | L77F |
| 298 | 279T > C | Silent |
| 311 | 292C > G | L98V |
| 334 | 315C > T | Silent |
| 388 | 369A > G | Silent |
| 559 | 540T > G | Silent |
| 564 | 545C > A | A182D |
| 607 | 588T > C | Silent |
| 644 | 625G > A | A209T |
| 646 | 627A > C | Silent |
| 679 | 660T > C | Silent |
| 688 | 669G > A | Silent |
| 704 | 685G > A | V229M |
| 721 | 702G > C | Silent |
| 724 | 705C > T | Silent |
| 730 | 711G > C | L237F |
| 800 | 781A > G | 3′ |

| | | | | |
|---|---|---|---|---|
| GPX1 | Y00433 | 138320 | GEN-TJ | Human mRNA for glutathione |

peroxidase (EC 1.11.1.9.)

| | | |
|---|---|---|
| 504 | 186G > A | Silent |
| 610 | 292C > G | R98G |
| 911 | 593C > T | P198L |
| 1048 | 730A > C | 3′ |
| 1110 | 792A > C | 3′ |

| | | | | |
|---|---|---|---|---|
| V00494 | V00494 | 103600 | GEN-TL | Human messenger RNA for serum |

albumin (HSA)

| | | |
|---|---|---|
| 34 | (−6)G > T | 5′ |
| 36 | (−4)C > G | 5′ |
| 401 | 362G > A | G121E |
| 431 | 392A > G | D131G |
| 1090 | 1051T > C | Silent |
| 1091 | 1052T > G | L351W |
| 1531 | 1492A > C | T498P |
| 1533 | 1494C > A | Silent |
| 1637 | 1598T > C | F533S |
| 1707 | 1668C > T | Silent |
| 1719 | 1680G > A | Silent |
| 1926 | 1887T > A | 3′ |

| | | | | |
|---|---|---|---|---|
| X00497 | X00497 | 142790 | GEN-TN | Human mRNA for HLA-DR antigens |

associated invariant chain (p33)

| | | |
|---|---|---|
| 805 | 750A > G | 3′ |
| 881 | 826A > G | 3′ |
| 1144 | 1089C > G | 3′ |

| | | | | |
|---|---|---|---|---|
| AJ004832 | AJ004832 | None | GEN-TO | Homo sapiens mRNA for neuropathy |

target esterase

| | | |
|---|---|---|
| 4153 | 3996G > A | 3′ |

| | | | | |
|---|---|---|---|---|
| HLA-DPB1 | X00532 | 142858 | GEN-U2 | Human mRNA for SB beta-chain |

(clone pII-beta-7)

| | | |
|---|---|---|
| 13 | 13T > A | S5T |
| 91 | 91T > A | S31T |
| 94 | 94C > T | R32C |
| 151 | 151C > A | L51M |
| 154 | 154C > A | Silent |
| 158 | 158G > C | S53T |
| 213 | 213G > A | Silent |
| 281 | 281C > T | T94M |
| 306 | 306C > T | Silent |
| 341 | 341A > G | Q114R |
| 353 | 353G > A | R118Q |
| 488 | 488C > T | T163M |
| 496 | 496T > C | S166P |
| 524 | 524C > T | T175I |
| 568 | 568A > G | R190G |
| 600 | 600G > A | Silent |
| 708 | 708C > G | 3′ |
| 761 | 761G > A | 3′ |
| 840 | 840G > A | 3′ |

| | | | | |
|---|---|---|---|---|
| SPINK1 | Y00705 | 167790 | GEN-UA | Homo sapiens pstI mPNA for |

pancreatic secretory inhibitor (expressed in neoplastic tissue)

| | | |
|---|---|---|
| 332 | 272C > T | 3′ |

| | | | | |
|---|---|---|---|---|
| TCRG | Y00790 | 186970 | GEN-UC | Human mRNA for T-cell receptor |

gamma-chain

| | | |
|---|---|---|
| 492 | 456G > A | Silent |
| 507 | 471A > G | Silent |
| 528 | 492C > T | Silent |
| 555 | 519A > T | Silent |
| 559 | 523A > G | I175V |
| 636 | 600C > T | Silent |
| 676 | 640G > A | E214K |
| 733 | 697A > G | I233V |
| 849 | 813G > T | W271C |
| 908 | 872C > T | T291M |
| 970 | 934A > G | R312G |

| | | | | |
|---|---|---|---|---|
| CBS | L00972 | 236200 | GEN-UV | Human cystathionine-beta |

synthase (CBS) mRNA

| | | |
|---|---|---|
| 1022 | 1022T > C | 3′ |
| 2001 | 2001C > T | 3′ |
| 2278 | 2278G > A | 3′ |
| 2358 | 2358G > C | 3′ |
| 2524 | 2524T > C | 3′ |
| 2545 | 2545C > T | 3′ |

| | | | | |
|---|---|---|---|---|
| AB005659 | AB005659 | None | GEN-VR | Homo sapiens SMRP mRNA, complete |
| cds | | | | |

| | | | | | |
|---|---|---|---|---|---|
| 1045C | 1781T | 2887C | 4158C | 4159G | 4820G |
| 1045T | 1781T | 4158C | 4159A | | |
| 1045C | 1781T | 2887T | 4l58C | 4l59A | 4820G |
| 1045C | 1781T | 2887C | 4158T | 4159G | 4820G |
| 1781G | 2887C | 4159G | | | |
| 1045C | 1781T | 2887C | 4158C | 4159G | 4820A |
| 1045C | 1781T | 2887C | 4158C | 4159A | 4820G |
| 1045T | 1781T | 2887C | 4158C | 4159G | 4820A |
| 1045C | 1781T | 2887T | 4158C | 4159G | |
| 1045C | 2887C | 4158C | 4159A | 4820A | |
| 1045C | 1781G | 2887C | 4158T | 4159G | |
| 1045C | 1781T | 2887C | 4158C | 4159A | 4820A |
| 1045T | 1781T | 4l58C | | | |
| 1045C | 1781T | 2887T | 4158C | 4159G | 4820A |
| 1045C | 1781T | 2887C | 4158C | 4159G | |
| 1045C | 1781T | 2887C | 4158T | 4159A | |
| 1045C | 1781T | 2887T | 4158C | 4159G | |
| 1045C | 1781T | 2887C | 4158C | 4159A | |
| 1045C | 2887C | 4158C | 4159A | 4820A | |

| | | |
|---|---|---|
| 1045 | 309C > T | Silent |
| 1781 | 1045T > G | S349A |
| 2887 | 2151T > C | Silent |
| 3642 | 2906C > T | 3′ |
| 4158 | 3422C > T | 3′ |
| 4159 | 3423A > G | 3′ |
| 4820 | 4084G > A | 3′ |

| | | | | |
|---|---|---|---|---|
| GSTM5 | L02321 | 138385 | GEN-WO | Human glutathione S-transferase |

(GSTM5) rnPNA, complete cds

| | | |
|---|---|---|
| 1406 | 1349T > C | 3′ |

| | | | | |
|---|---|---|---|---|
| 10 X02812 | X02812 | 190180 | GEN-XR | Human mRNA for transforming |

growth factor-beta (TGF-beta)

| | | |
|---|---|---|
| 870T | 979C | 1854C |
| 979G | 1854G | |
| 870C | 979C | 1854G |
| 870T | 979C | 1854G |
| 870C | 979G | 1854G |
| 870T | 979C | |

| | | |
|---|---|---|
| 870 | 29C > T | P10L |
| 979 | 138C > G | I46M |
| 1632 | 791C > T | T264I |
| 1807 | 966C > T | Silent |
| 1854 | 1013G > C | S338T |
| 1930 | 1089G > A | Silent |
| 1942 | 1101C > T | Silent |
| 2013 | 1172G > A | S391N |

| | | | | |
|---|---|---|---|---|
| NMOR2 | J02888 | 160998 | GEN-XT | Human quinone oxidoreductase |

(NQO2) mRNA, complete cds

| | | |
|---|---|---|
| 505 | 330G > A | Silent |
| 909 | 734G > C | 3′ |

| | | | | |
|---|---|---|---|---|
| CBG | J02943 | 122500 | GEN-Y2 | Human corticosteroid binding |

globulin mRNA, complete cds

| | | |
|---|---|---|
| 106 | 71A > T | D24V |
| 413 | 378T > C | Silent |
| 971 | 936T > C | Silent |
| 1229 | 1194G > A | Silent |

| | | | | |
|---|---|---|---|---|
| SOD3 | J02947 | 185490 | GEN-Y3 | Human extracellular-superoxide |

dismutase (SOD3) mPNA, complete cds

| | | |
|---|---|---|
| 746 | 677C > A | Frame |
| 1042 | 973C > T | 3′ |

| | | | | |
|---|---|---|---|---|
| HLA-DOB | X03066 | 600629 | GEN-ZO | Human mRNA for HLA-D class II |

antigen DO beta chain

| | | |
|---|---|---|
| 32 | (−25)G > A | 5′ |
| 1147 | 1091C > T | 3′ |
| 1299 | 1243A > G | 3′ |

| | | | | |
|---|---|---|---|---|
| J03143 | J03143 | 107470 | GEN-ZK | Human interferon-gamma receptor |

mRNA, complete cds

| | | |
|---|---|---|
| 1098 | 1050T > G | Silent |

| | | | | |
|---|---|---|---|---|
| K03195 | K03195 | 138140 | GEN-ZT | Human (HepG2) glucose |

transporter gene mRNA, complete cds

| | | |
|---|---|---|
| 1484 | 1305C > T | Silent |
| 2120 | 1941G > C | 3′ |

| | | | | |
|---|---|---|---|---|
| LIPC | J03540 | 151670 | GEN-11J | Human hepatic lipase mRNA, |

complete cds

| | | | |
|---|---|---|---|
| 469 | 465T > G | | Silent |
| 595 | 591A > G | Silent | |
| 648 | 644G > A | S215N | |
| 817 | 813C > T | Silent | |
| 1441 | 1437C > A | Silent | |

| | | | | |
|---|---|---|---|---|
| J03548 | J03548 | 103260 | GEN-11M | Human adrenodoxin mRNA, complete |
| cds | | | | |

| | | |
|---|---|---|
| 1099 | 967G > A | 3′ |
| 1123 | 991T > C | 3′ |
| 1222 | 1090G > C | 3′ |
| 1254 | 1122G > A | 3′ |

| | | | | |
|---|---|---|---|---|
| CYP1B1 | U03688 | 601771 | GEN-11Y | Human dioxin-inducible |

cytochrome P450 (CYP1Bl) mPNA, complete cds

| | | |
|---|---|---|
| 488 | 142C > G | R48G |
| 701 | 355G > T | A119S |
| 2673 | 2327G > T | 3′ |

| | | | | |
|---|---|---|---|---|
| J03746 | J03746 | 138330 | GEN-11Z | Human glutathione S-transferase |

mRNA, complete ccis

| | | |
|---|---|---|
| 560G | 598T | 676T |
| 560G | 598T | 676C |
| 560A | 598T | 676T |
| 560G | 598G | 676T |

| | | |
|---|---|---|
| 560 | 487A > G | 3′ |
| 598 | 525T > G | 3′ |
| 676 | 603T > C | 3′ |

| | | | | |
|---|---|---|---|---|
| PNMT | J03727 | 171190 | GEN-120 | Human phenylethanolamine N- |

methyltransferase mRNA,complete cds

| | | |
|---|---|---|
| 462 | 456A > G | Silent |

| | | | | |
|---|---|---|---|---|
| AB007448 | AB007448 | None | GEN-125 | Homo sapiens mRNA for |

polyspecific oraganic cation transporter, complete cds

| | | |
|---|---|---|
| 1559 | 1413C > G | Silent |

| | | | | |
|---|---|---|---|---|
| NNOR1 | J03934 | 125860 | GEN-12L | Human, NAD(P)H:menadione |

oxidoreductase mRNA, complete cds

| | |
|---|---|
| 609C | 1994T |
| 609T | 1994C |
| 609C | 1994C |
| 609T | 1994T |

| | | |
|---|---|---|
| 609 | 559C > T | P187S |
| 1784 | 1734T > G | 3′ |
| 1994 | 1944C > T | 3′ |

| | | | | |
|---|---|---|---|---|
| AF007216 | AF007216 | 603345 | GEN-13L | Homo sapiens sodium bicarbonate |

cotransporter (HNBC1) mRNA, complete cds

| | | | | | |
|---|---|---|---|---|---|
| 3332A | 3666C | 4194C | 4240T | 4633C | 5283A |
| 3332A | 3666C | 4194G | 4633C | | |
| 3332C | 3666C | 4194C | 4240T | 4633G | 5283A |
| 3332A | 3666G | 4194G | 4633G | 5283G | |
| 3332A | 3666C | 4194G | 4240T | 4633G | 5283A |
| 3332A | 3666C | 4194C | 4633G | 5283A | |
| 3332A | 3666C | 4194C | 4240T | 4633G | 5283A |
| 3332A | 3666C | 4194G | 4240A | 4633G | 5283A |
| 3666C | 4194C | 4240A | 4633G | 5283A | |
| 3332A | 3666G | 4194C | 4240T | | |
| 3332A | 3666G | 4194C | 4240T | 4633C | 5283A |
| 3666C | 4194C | 4240T | 4633G | 5283A | |
| 3332A | 3666C | 4194C | 4240A | 4633G | 5283A |
| 3332A | 3666G | 4194G | 4240A | 5283A | |
| 3332A | 3666C | 4194G | | | |
| 3666C | 4194G | 4240T | 4633G | 5283G | |
| 3332C | 3666G | 4194C | 4240T | 4633G | 5283A |
| 3666C | 4194G | 4240T | 4633C | 5283A | |
| 3332A | 3666C | 4194C | 4240T | 5283A | |
| 3332A | 3666G | 4194G | 4240T | 4633G | 5283G |
| 3332C | 3666C | 4194C | 4240A | 4633G | 5283A |
| 3332A | 3666G | 4194C | 4240T | | |
| 3332A | 3666C | 4194G | 4240T | 4633G | |
| 3332C | 3666C | 4194C | 4240A | 5283A | |

| | | |
|---|---|---|
| 3332 | 3183C > A | 3′ |
| 3666 | 3517C > G | 3′ |
| 4194 | 4045C > G | 3′ |
| 4240 | 4091T > A | 3′ |
| 4633 | 4484G > C | 3′ |
| 5283 | 5134A > G | 3′ |

| | | | | |
|---|---|---|---|---|
| OBR | J04056 | 114830 | GEN-13O | Human carbonyl reductase mRNA, |

complete cds

| | | |
|---|---|---|
| 1060 | 9670 > A | 3′ |

| | | | | |
|---|---|---|---|---|
| CAT | X04076 | 115500 | GEN-13P | Human kidney mRNA for catalase |

| | | | |
|---|---|---|---|
| 796C | 1237C | 1325C | 1387T |
| 796C | 1237T | 1325T | 1387C |
| 796A | 1237C | 1325C | 1387C |
| 796C | 1237T | 1325C | 1387C |
| 796C | 1237C | 1325T | 1387C |
| 796C | 1237C | 1325C | 1387C |

| | | |
|---|---|---|
| 51 | (−20)T > C | 5′ |
| 218 | 148C > T | L50F |
| 796 | 726C > A | Silent |
| 1237 | 11671 > C | Silent |
| 1325 | 1255C > T | Silent |
| 1387 | 1317C > T | Silent |
| 2131 | 2061A > C | 3′ |

| | | | | |
|---|---|---|---|---|
| AB000812 | AB000812 | 602550 | GEN-14E | Human mRNA for BMAL1b, complete |
| cds | | | | |

| | | |
|---|---|---|
| 1084 | 1044C > A | Silent |

| | | | | |
|---|---|---|---|---|
| G22P1 | J04611 | 152690 | GEN-153 | Human lupus p70 (Ku) autoantigen |

protein mRNA, complete cds

| | | |
|---|---|---|
| 1762 | 1729A > T | T577S |
| 1812 | 1779T > G | Silent |
| 1900 | 1867G > T | 3′ |

| | | | | |
|---|---|---|---|---|
| HADHA | U04627 | 600890 | GEN-155 | Human 78 kDa gastrin-binding |

protein mRNA, complete cds

| | | |
|---|---|---|
| 474 | 474C > T | Silent |
| 1507 | 1507G > A | V503M |

| | | | | |
|---|---|---|---|---|
| L04751 | L04751 | 601310 | GEN-157 | Human cytochrome p-450 4A |

(CYP4A) mRNA, complete cds

| | | |
|---|---|---|
| 1001 | 969C > T | Silent |
| 1333 | 1301T > C | F434S |
| 1406 | 1374T > C | Silent |
| 1944 | 1912A > G | 3′ |
| 1970 | 1938G > A | 3′ |
| 2011 | 1979C > T | 3′ |
| 2047 | 2015T > C | 3′ |
| 2115 | 2083A > G | 3′ |

| | | | | |
|---|---|---|---|---|
| RORA | U04897 | 600825 | GEN-15R | Human orphan hormone nuclear |

receptor RORalpha1 mRNA, complete cds

| | | |
|---|---|---|
| 884T | 1527A | 1529A |
| 884C | 1527G | 1529A |
| 884C | 1527A | 1529A |
| 884C | 1527A | 1529C |

| | | |
|---|---|---|
| 884 | 783C > T | Silent |
| 1527 | 1426A > G | T476A |
| 1529 | 1428A > C | Silent |

| | | | | |
|---|---|---|---|---|
| GSTM3 | J05459 | 138390 | GEN-17O | Human glutathione transferase M3 |

(GSTM3) mRNA, complete cds

| | | |
|---|---|---|
| 687 | 670G > A | V224I |

| | | | | |
|---|---|---|---|---|
| AJ001838 | AJ001838 | 603758 | GEN-17S | Homo sapiens mRNA for |

maleylacetoacetate isomerase

| | | |
|---|---|---|
| 65 | (−39)G > C | 5′ |
| 197 | 94A > G | K32E |
| 227 | 124G > A | G42R |
| 348 | 245C > T | T82M |

| | | | | |
|---|---|---|---|---|
| AF001945 | AF001945 | 601691 | GEN-17Z | Homo sapiens rim ABC transporter |

(ABCR) mRNA, complete cds

| | | |
|---|---|---|
| 2725 | 2644G > A | G882S |
| 5136 | 5055C > T | Silent |

| | | | | |
|---|---|---|---|---|
| DDH1 | U05598 | 600450 | GEN-184 | Human dihydrodiol dehydrogenase |

mRNA, complete cds

| | | |
|---|---|---|
| 139A | 179T | 806A |
| 139G | 179T | |
| 139A | 179A | 806G |
| 139A | 179T | 806G |
| 139G | 179T | 806G |

| | | |
|---|---|---|
| 38 | 15C > T | Silent |
| 139 | 116A > G | K39R |
| 179 | 156A > T | Silent |
| 282 | 259A > T | S87C |
| 350 | 327C > T | Silent |
| 365 | 342T > C | Silent |
| 464 | 44lG > A | Silent |
| 474 | 451A > G | M151V |
| 532 | 509A > G | H170R |
| 538 | 515T > A | L172Q |
| 689 | 666T > C | Silent |
| 806 | 783G > A | Silent |
| 872 | 849G > T | Silent |
| 952 | 929T > G | I310S |
| 1020 | 997G > A | 3′ |
| 1035 | 1012G > A | 3′ |
| 1112 | 1089C > T | 3′ |

| | | | | |
|---|---|---|---|---|
| EPHX2 | L05779 | 132811 | GEN-18A | Human cytosolic epoxide |

hydrolase mRNA, complete cds

| | | | | | | | | | |
|---|---|---|---|---|---|---|---|---|---|
| 205A | 348C | 502G | 632A | 898G | 1274C | 1631C | 1742A | 1800T | |
| 205A | 348C | 502G | 632A | 898G | 1274C | 1313G | 1631A | 1742A | 1800T |
| 205A | 348C | 502G | 632A | 898A | 1274C | 1313G | 1631C | 1742G | 1800C |
| 205A | 348C | 502G | 632A | 898G | 1274C | 1313A | 1631A | 1742A | 1800T |
| 205A | 348C | 502G | 632A | 898G | 1274C | 1313A | 1631C | 1742G | 1800C |
| 205G | 348C | 502G | 898G | 1274C | 1313G | 1631A | 1742A | 1800T | |
| 205A | 348T | 502G | 632A | 898G | 1274C | 1313G | 1631A | 1742A | 1800T |
| 205A | 348C | 502G | 632A | 898A | 1274C | 1313G | 1631C | 1742G | 1800T |
| 205G | 348C | 502G | 632A | 898G | 1274C | 1313G | 1631C | 1742G | 1800C |
| 205A | 348C | 502A | 632A | 898G | 1274C | 1313G | 1631A | 1742A | 1800T |
| 348C | 502G | 632A | 898G | 1274T | 1313G | 1631C | 1742A | | |
| 205A | 348C | 502G | 632C | 898G | 1274C | 1313G | 1631C | 1742G | 1800C |
| 205G | 348C | 502G | 632A | 898G | 1274T | 1313G | 1631C | 1742G | 1800C |
| 205A | 348C | 502G | 632A | 898G | 1274C | 1313G | 1631C | 1742G | |
| 205A | 348C | 502G | 632A | 898G | 1313G | 1631C | 1742G | 1800T | |
| 205G | 348C | 502G | 632A | 898G | 1631A | 1742G | 1800C | | |
| 205A | 348C | 502G | 632A | 898G | 1274C | 1313G | 1742A | 1800T | |
| 205A | 348C | 502G | 632A | 898G | 1274T | 1313G | 1631C | 1742A | 1800T |
| 205A | 348C | 502G | 632A | 898G | 1274C | 1313G | 1631C | 1742G | 1800C |
| 205A | 348C | 502G | 632A | 898G | 1274C | 1313A | 1631A | 1742G | 1800C |
| 205A | 348C | 502G | 632A | 898G | 1313G | 1631C | 1742G | 1800C | |
| 205G | 348C | 502G | 632C | 898G | 1274C | 1313G | 1631A | 1742A | 1800T |
| 205A | 348C | 502G | 632C | 898A | 1274C | 1313G | 1631A | 1742G | 1800C |

| | | |
|---|---|---|
| 205 | 164A > G | K55R |
| 348 | 307C > T | R103C |
| 502 | 461G > A | C154Y |
| 632 | 591A > C | Silent |
| 898 | 857G > A | R286Q |
| 1274 | 1233C > T | Silent |
| 1313 | 1272G > A | Silent |
| 1631 | 1590A > C | Silent |
| 1742 | 1701A > G | 3′ |
| 1800 | 1759T > C | 3′ |

| | | | | |
|---|---|---|---|---|
| U05875 | U05875 | 147569 | GEN-18J | Human clone pSK1 interferon |

gamma receptor accessory factor-1 (AF-1) mRNA, complete cds

| | | | | | |
|---|---|---|---|---|---|
| 520C | 685C | 821C | 839A | 1192A | 1664C |
| 520C | 685C | 821C | 839A | 1192A | 1664T |
| 520C | 685C | 821C | 839A | 1192A | 1664C |
| 520C | 685T | 821C | 839A | 1192A | 1664C |
| 520C | 685C | 821C | 839G | 1192A | 1664C |
| 520T | 685C | 821C | 839A | 1192A | 1664C |
| 520C | 685C | 1192G | 1664C | | |
| 520C | 685C | 821G | 839A | 1192A | 1664T |
| 520C | 685C | 821G | 839A | 1192G | 1664C |
| 520T | 685C | 821G | 839A | 1192G | 1664C |
| 520T | 685C | 821G | 839A | 1192A | 1664C |

| | | |
|---|---|---|
| 520 | (−129)C > T | 5′ |
| 685 | 37C > T | L13F |
| 821 | 173C > G | T58R |
| 839 | 191G > A | R64Q |
| 1192 | 544A > G | K182E |
| 1664 | 1016C > T | 3′ |
| 2047 | 1399C > G | 3′ |
| 2087 | 1439T > C | 3′ |

| | | | | |
|---|---|---|---|---|
| XDH | U06117 | 278300 | GEN-194 | Human xanthine dehydrogenase |

(XDH) mRNA, complete cds

| | | |
|---|---|---|
| 3951 | 3888C > G | Silent |

| | | | | |
|---|---|---|---|---|
| TCRD | X06557 | 186810 | GEN-19M | Human mRNA for TCR-delta chain |

| | | |
|---|---|---|
| 1032 | 1014C > A | 3′ |

| | | | | |
|---|---|---|---|---|
| GSTP1 | X06547 | 134660 | GEN-19N | Human mRNA for class Pi |

glutathione S-transferase (GST-Pi; E.C.2.5.1.18)

| | | |
|---|---|---|
| 156 | 150C > T | Silent |
| 319 | 313A > G | I105V |
| 347 | 341C > T | A114V |
| 561 | 555C > T | Silent |

| | | | | |
|---|---|---|---|---|
| EHHADH | L07077 | 261515 | GEN-1DF | Human enyol-CoA: hydratase 3- |

hydroxyacyl-CoA dehydrogenase (EHHADH) mRNA, complete cds with repeats

| | | | | | | |
|---|---|---|---|---|---|---|
| 1812G | 2060A | | | | | |
| 1812G | 2060C | | | | | |
| 1812A | 2060A | | | | | |
| 2151T | 2240G | 2700T | 2960G | | | |
| 1812G | 2060A | 2151C | 2240G | 2700T | 2960G | 3268A |
| 1812A | 2060A | 2151T | 2240A | 2700G | 2960A | 3268A |
| 1812G | 2060A | 2151C | 2240G | 2700G | 2960G | 3268A |

| | | |
|---|---|---|
| 1225 | 1218G > A | Silent |
| 1812 | 1805G > A | R602Q |
| 1823 | 1816C > A | P606T |
| 2060 | 2053C > A | Q685K |
| 2151 | 2144T > C | L715S |
| 2240 | 2233G > A | 3′ |
| 2700 | 2693T > G | 3′ |
| 2960 | 2953G > A | 3′ |
| 3268 | 3261A > G | 3′ |

| | | | | |
|---|---|---|---|---|
| L07592 | L07592 | 600409 | GEN-1E7 | Human peroxisome proliferator |

activated receptor mRNA, complete cds

| | | | | | | | | |
|---|---|---|---|---|---|---|---|---|
| 251T | 271G | 317G | 826T | 1821T | 2359C | 2960C | | |
| 251C | 271G | 317G | 826C | 1228C | 2359C | 2926A | | |
| 251C | 271G | 317G | 826C | 1228C | 1821T | 2359C | 2926G | |
| 251C | 271G | 317G | 826C | 1228C | 1821T | 2359T | 2926G | |
| 251C | 271G | 317T | 826C | 1228C | 2359C | 2926A | | |
| 251T | 271G | 317G | 826T | 1228C | 1821C | 2359C | 2926G | 2960T |
| 251T | 271A | 317G | 826T | 1228C | 1821T | 2926G | 2960T | |
| 251T | 271G | 317G | 826C | 1821T | 2359C | 2926A | | |
| 251T | 271G | 317G | 826T | 1228C | 1821T | 2359C | 2926G | 2960T |
| 251T | 271G | 317G | 826T | 1228C | 1821T | 2359T | 2926G | 2960T |
| 251T | 271A | 317G | 826T | 1228T | 1821T | 2359C | 2926G | |
| 251T | 271G | 317G | 826T | 1821T | 2359C | 2926A | 2960G | |
| 251C | 271G | 317G | 826C | 1228C | 1821T | 2359T | 2926G | 2960T |
| 251C | 271G | 317G | 826C | 1228C | 2359C | 2926A | | |
| 251C | 271G | 317G | 826C | 1821T | 2359C | 2926G | | |
| 251C | 271G | 317G | 826C | 1228C | 1821T | 2359C | 2926G | 2960C |
| 251C | 271G | 317G | 826C | 1228C | 1821T | 2359C | 2926G | |
| 251C | 271G | 317T | 826C | 1228C | 2359C | 2926A | | |
| 251C | 271G | 317G | 826C | 1228C | 2359C | 2926G | | |
| 251C | 271G | 317G | 1821T | 2359C | 2926A | | | |
| 251T | 271A | 317G | 826T | 1821T | 2359C | 2926A | | |
| 251C | 271G | 317G | 1228C | 1821T | 2359C | 2926G | 2960C | |
| 251C | 271G | 317G | 826C | 1228C | 2359C | 2926G | 2960C | |
| 251T | 271G | 317G | 826C | 1821T | 2359C | 2926A | | |
| 251C | 271G | 317G | 826C | 1228C | 1821T | 2359T | 2926G | 2960C |
| 251T | 271G | 317G | 1228C | 1821T | 2359C | 2926A | | |
| 251T | 271A | 317G | 826T | 1228C | 1821T | 2359C | 2926G | 2960T |

| | | |
|---|---|---|
| 251 | (−87)C > T | 5′ |
| 271 | (−67)G > A | 5′ |
| 317 | (−21)G > T | 5′ |
| 826 | 489C > T | Silent |
| 1228 | 891C > T | Silent |
| 1821 | 1484T > C | 3′ |
| 2359 | 2022C > T | 3′ |
| 2926 | 2589A > G | 3′ |
| 2960 | 2623T > C | 3′ |
| 3119 | 2782C > G | 3′ |

| | | | | |
|---|---|---|---|---|
| TGFER3 | L07594 | 600742 | GEN-1EA | Human transforming growth |

factor-beta type III receptor (TGF-beta) mRNA, complete cds

| | | |
|---|---|---|
| 1548 | 1200G > A | Silent |
| 2370 | 2022C > T | Silent |
| 3966 | 3618G > C | 3′ |

| | | | | |
|---|---|---|---|---|
| SOD2 | X07834 | 147460 | GEN-1ES | Human mRNA for manganese |

superoxide dismutase (EC 1.15.1.1)

| | | |
|---|---|---|
| 44 | 40C > G | P14A |
| 51 | 47T > C | V16A |
| 198 | 194C > A | T65N |
| 249 | 245T > C | I82T |

| | | | | |
|---|---|---|---|---|
| ALDH6 | U07919 | 600463 | GEN-1F5 | Human aldehyde dehydrogenase 6 |

mRNA, complete cds

| | | |
|---|---|---|
| 2453 | 2401A > G | 3′ |
| 3396 | 3344C > T | 3′ |
| 3397 | 3345G > A | 3′ |

| | | | | |
|---|---|---|---|---|
| LCT | X07994 | 603202 | GEN-1F6 | Human mRNA for lactase-phiorizin |

hydrolase LPH (EC 3.2.1.23-62)

| | | |
|---|---|---|
| 5845 | 5834C > G | 3′ |

| | | | | |
|---|---|---|---|---|
| AB003791 | AB003791 | 603797 | GEN-1F9 | Homo sapiens mRNA for keratan |

sulfate Gal-6-sulfotransferase, complete cds

| | | |
|---|---|---|
| 1617 | 1251G > A | 3′ |
| 1643 | 1277G > A | 3′ |

| | | | | |
|---|---|---|---|---|
| U08015 | U08015 | 600489 | GEN-1FD | Human NF-ATc mRNA, complete cds |

| | | |
|---|---|---|
| 530 | 291C > T | Silent |
| 1094 | 855G > A | Silent |
| 2222 | 1983G > A | Silent |
| 2225 | 1986A > G | Silent |
| 2295 | 2056A > C | S686R |

| | | | | |
|---|---|---|---|---|
| X08006 | X08006 | 124030 | GEN-1FE | Human mRNA for cytochrome P450 |
| db1 | | | | |

| | | | | | | |
|---|---|---|---|---|---|---|
| 100C | 281A | 336C | 635A | | | |
| 100T | 281A | 336T | 386C | 635G | 886C | 1457C |
| 100C | 281A | 336C | 386G | 635G | 886C | 1457G |
| 100T | 336C | 386C | 635G | 886C | 1457C | |
| 100C | 281A | 336C | 386C | 635G | 886T | 1457C |
| 100C | 281A | 336C | 386C | 635A | 886T | 1457C |
| 100T | 281G | 336C | 386C | 635G | 886C | 1457C |

| | | |
|---|---|---|
| 100 | 100C > T | P34S |
| 281 | 281A > G | H94R |
| 336 | 336C > T | Silent |
| 386 | 386G > C | R129P |
| 408 | 408G > C | Silent |
| 454 | 454delT | Frame |
| 635 | 635G > A | G212E |
| 692 | 692T > C | L231P |
| 696 | 696T > C | Silent |
| 775 | 775delA | Frame |
| 801 | 801C > A | Silent |
| 836 | 836T > A | M279K |
| 854 | 854A > G | N2855 |
| 886 | 886C > T | R296C |
| 1108 | 1108G > A | V370I |
| 1401 | 1401G > C | Silent |
| 1457 | 1457G > C | S486T |

| | | | | |
|---|---|---|---|---|
| U08021 | U08021 | 600008 | GEN-1FG | Human nicotinamide N- |

methyltransferase (NNMT) mPNA, complete cds

| | | |
|---|---|---|
| 584 | 467C > G | P156R |
| 613 | 496C > T | Silent |

| | | | | |
|---|---|---|---|---|
| CCKBR | L08112 | 118445 | GEN-1FL | Cholecystokinin-B/gastrin |
| receptor | | | | |

| | | |
|---|---|---|
| 456 | 456G > A | Silent |

| | | | | |
|---|---|---|---|---|
| FACL1 | L09229 | 152425 | GEN-1GI | Human long-chain acyl-coenzyme A |

synthetase (FACL1) mRNA, complete cds

| | |
|---|---|
| 487C | 1648A |
| 487A | 1648G |
| 487C | 1648G |

| | | |
|---|---|---|
| 487 | 414C > A | Silent |
| 1648 | 1575G > A | Silent |
| 3026 | 2953G > A | 3′ |
| 3083 | 3010G > A | 3′ |

| | | | | |
|---|---|---|---|---|
| AF009746 | AF009746 | 603214 | GEN-1HZ | Homo sapiens peroxisomal |

membrane protein 69 (PMP69) mRNA, complete cds

| | | |
|---|---|---|
| 961 | 910G > A | A304T |
| 1895 | 1844A > G | 3′ |
| 2134 | 2083T > G | 3′ |

| | | | | |
|---|---|---|---|---|
| FABP2 | M10050 | 134640 | GEN-1IE | Human liver fatty acid binding |

protein (FABP) mRNA, complete cds

| | | |
|---|---|---|
| 322 | 280G > A | A94T |

| | | | | |
|---|---|---|---|---|
| Y10387 | Y10387 | None | GEN-1IU | H. sapiens mRNA for PAPS |

synthetase

| | | |
|---|---|---|
| 55 | 19T > C | Silent |
| 999 | 963C > T | Silent |
| 1981 | 1945G > A | 3′ |

| | | | | |
|---|---|---|---|---|
| Y10659 | Y10659 | 300119 | GEN-1J6 | H. sapiens IL-13Ra mRNA |

| | |
|---|---|
| 1408A | 1508G |
| 1408A | 1508A |
| 1408G | 1508G |

| | | |
|---|---|---|
| 1116 | 1073G > A | G358D |
| 1408 | 1365A > G | 3′ |
| 1508 | 1465G > A | 3′ |
| 1685 | 1642A > G | 3′ |
| 1889 | 1846C > T | 3′ |

| | | | | |
|---|---|---|---|---|
| U10868 | U10868 | 600466 | GEN-1JF | Human aldehyde dehydrogenase |

ALDH7 mRNA, complete cds

| | | |
|---|---|---|
| 2681 | 2634T > C | 3′ |

| | | | | |
|---|---|---|---|---|
| L21005 | L11005 | 602841 | GEN-1JU | Human aldehyde oxidase (hAOX) |

mRNA, complete cds

| | | | | | | |
|---|---|---|---|---|---|---|
| 1721A | 2331T | 2341A | 3534A | 3865C | | |
| 1721A | 2331T | 2341G | 3534A | 3865C | | |
| 1721T | 2331T | 2341A | 3195T | 3534A | 3865C | |
| 1721T | 2331T | 2341G | 3195C | 3250C | 3534A | 3865C |
| 1721T | 2331T | 3195C | 3250C | 3865T | | |
| 1721T | 2331T | 2341A | 3195C | 3250C | 3534G | 3865C |
| 1721T | 2331T | 2341A | 3195C | 3250C | 3534A | 3865C |
| 1721T | 2331G | 2341A | 3534A | 3865C | | |
| 1721T | 2331G | 2341A | | | | |
| 1721A | 2331T | 2341G | 3534A | 3865C | | |
| 1721T | 2331T | 2341A | 3195T | 3250T | 3534A | 3865C |
| 1721T | 2331T | 2341G | | | | |
| 1721T | 2331T | 2341G | 3195C | 3250C | 3534G | 3865T |
| 1721A | 2331T | 2341A | 3534A | 3865C | | |

| | | |
|---|---|---|
| 1721 | 1591T > A | S531T |
| 2331 | 2201T > G | V734G |
| 2341 | 2211A > G | Silent |
| 3195 | 3065C > T | A1022V |
| 3250 | 3120C > T | Silent |
| 3534 | 3404A > G | N1135S |
| 3865 | 3735C > T | Silent |
| 4284 | 4154C > A | 3′ |
| 4447 | 4317G > C | 3′ |
| 4525 | 4395T > G | 3′ |
| 4675 | 4545G > A | 3′ |

| | | | | |
|---|---|---|---|---|
| ADH3 | M12272 | 103730 | GEN-1LU | Homo sapiens alcohol |

dehydrogenase class I gamma subunit (ADH3) mRNA, complete cds

| | | |
|---|---|---|
| 1128 | 1048A > G | I350V |

| | | | | |
|---|---|---|---|---|
| TPMT | U12387 | 187680 | GEN-1LY | Human thiopurine |

methyltransferase (TPMT) mRNA, complete cds

| | | |
|---|---|---|
| 536G | 795A | 1085C |
| 536G | 795A | 1085T |
| 536G | 795G | 1085T |
| 536A | 1085T | |
| 536A | 795G | 1085T |
| 536G | 795A | |

| | | |
|---|---|---|
| 536 | 460G > A | A154T |
| 795 | 719A > G | Y240C |
| 1085 | 1009T > C | 3′ |
| 1336 | 1260C > T | 3′ |
| 1373 | 1297G > A | 3′ |

| | | | | |
|---|---|---|---|---|
| X12387 | X12387 | 124010 | GEN-1LZ | Cytochrome P-450, CYP3A4 |

| | | |
|---|---|---|
| 1751T | 1847C | 2525A |
| 1751T | 1847A | 2525A |
| 1751T | 1847C | 2525T |
| 1751A | 1847C | 2525A |

| | | |
|---|---|---|
| 44 | (−26)G > C | 5′ |
| 628 | 559A > T | T187S |
| 646 | 577A > G | I193V |
| 676 | 607T > C | F203L |
| 823 | 754T > G | S252A |
| 1361 | 1292T > C | I431T |
| 1751 | 1682T > A | 3′ |
| 1847 | 1778C > A | 3′ |
| 2189 | 2120G > A | 3′ |
| 2525 | 2456A > T | 3′ |

| | | | | |
|---|---|---|---|---|
| X22530 | X12530 | 112210 | GEN-1MH | Human mRNA for B lymphocyte |

antigen CD20 (B1, Bp35)

| | |
|---|---|
| 309C | 1318A |
| 309T | 1318G |
| 309C | 1318G |

| | | |
|---|---|---|
| 131 | 38C > T | P13L |
| 309 | 216C > T | Silent |
| 1318 | 1225G > A | 3′ |

| | | | | |
|---|---|---|---|---|
| GC | M12654 | 139200 | GEN-1MN | Human serum vitamin D-binding |

protein (hDBP) mRNA, complete cds

| | | |
|---|---|---|
| 925 | 897T > C | Silent |
| 1324 | 1296G > T | E432D |
| 1335 | 1307C > A | T436K |
| 1362 | 1334G > A | R445H |

| | | | | |
|---|---|---|---|---|
| D13138 | D13138 | 179780 | GEN-1NW | Human mRNA for dipeptidase |

| | | |
|---|---|---|
| 566 | 523T > G | S175A |

| | | | | |
|---|---|---|---|---|
| CRYZ | L13278 | 123691 | GEN-1NZ | Homo sapiens zeta |

crystallin/quinone reductase mRNA, complete cds

| | | |
|---|---|---|
| 64 | 54G > A | Silent |
| 902 | 892G > A | V298M |
| 1229 | 1219A > G | 3′ |

| | | | | |
|---|---|---|---|---|
| L13286 | L13286 | 600125 | GEN-103 | Human mitochondrial 1,25- |

dihydroxyvitamin D3 24-hydroxylase mRNA, complete cds

| | | |
|---|---|---|
| 2031 | 1638G > A | 3′ |

| | | | | |
|---|---|---|---|---|
| MDCR | L13385 | 601545 | GEN-106 | Homo sapiens (clone 71) Miller- |

Dieker lissencephaly protein (LIS1) mRNA, complete cds

| | | |
|---|---|---|
| 1467 | 1250C > T | 3′ |
| 1868 | 1651C > T | 3′ |
| 1917 | 1700C > T | 3′ |
| 2962 | 2745G > T | 3′ |
| 4589 | 4372G > A | 3′ |

| | | | | |
|---|---|---|---|---|
| X13561 | X13561 | 147910 | GEN-10H | Human mRNA for preprokallikrein |

(EC 3.4.21)

| | | |
|---|---|---|
| 592A | 603T | 732C |
| 603C | 732T | |
| 592G | 603C | 732C |
| 592A | 603C | 732C |
| 592G | 603C | 732T |

| | | |
|---|---|---|
| 54 | 18G > T | Silent |
| 441 | 405T > C | Silent |
| 469 | 433G > C | E145Q |
| 592 | 556A > G | K186E |
| 603 | 567C > T | Silent |
| 732 | 696C > T | Silent |

| | | | | |
|---|---|---|---|---|
| ORM1 | M13692 | 138600 | GEN-1P5 | Human alpha-1 acid glycoprotein |

mRNA, complete cds

| | | |
|---|---|---|
| 128 | 113A > G | Q38R |
| 222 | 207C > T | Silent |
| 273 | 258A > C | Silent |
| 296 | 281C > A | T94N |
| 514 | 499C > T | R167C |
| 535 | 520G > A | V174M |
| 654 | 639G > T | 3′ |

| | | | | |
|---|---|---|---|---|
| X13930 | X13930 | 122720 | GEN-1Q3 | Human CYP2A4 mRNA for P-450 IIA4 |
| protein | | | | |

| | | |
|---|---|---|
| 60 | 51A > G | Silent |
| 253 | 246T > C | Silent |
| 272 | 263G > A | R88K |
| 1072 | 1063G > A | V355M |
| 1146 | 1137G > A | Silent |
| 1485 | 1476G > T | Silent |
| 1675 | 1666A > T | 3′ |
| 1677 | 1668C > G | 3′ |
| 1697 | 1688C > A | 3′ |

| | | | | |
|---|---|---|---|---|
| TBG | M14091 | 314200 | GEN-1QO | Human thyroxine-binding globulin |

mRNA, complete cds

| | | |
|---|---|---|
| 901 | 571G > A | D191N |
| 1239 | 909G > T | L303F |

| | | | | |
|---|---|---|---|---|
| ALPL | X14174 | 171760 | GEN-1QR | Human mRNA for liver-type |

alkaline phosphatase (EC 3.1.3.1)

| | | | |
|---|---|---|---|
| 730C | 1187T | 1276G | |
| 730C | 1187T | 1276A | 2040C |
| 730T | 1187T | 1276A | 2040T |
| 730C | 1187T | 1276A | 2040T |
| 730C | 1187C | 1276A | 2040C |
| 730C | 1187C | 1276G | |
| 730C | 1276G | | |
| 730C | 1187C | 1276G | 2040T |
| 730T | 1187C | 1276G | |
| 730C | 1187C | 1276G | |

| | | |
|---|---|---|
| 730 | 330C > T | Silent |
| 1187 | 787C > T | H263Y |
| 1276 | 876G > A | Silent |
| 2040 | 1640C > T | 3′ |

| | | | | |
|---|---|---|---|---|
| U14510 | U14510 | 602698 | GEN-1RD | Human transcription factor NFATx |

mRNA, complete cds

| | | |
|---|---|---|
| 2128 | 2104A > C | M702L |
| 2516 | 2492T > G | L831W |
| 2720 | 2696C > G | A899G |
| 2792 | 2768C > T | A923V |
| 2828 | 2804C > G | A935G |
| 2903 | 2879C > G | A960G |
| 2967 | 2943G > A | Silent |
| 3333 | 3309G > A | 3′ |
| 3577 | 3553G > A | 3′ |

| | | | | |
|---|---|---|---|---|
| SLC1A4 | L14595 | 600229 | GEN-1RI | Human |

alanine/serine/cysteine/threonine transporter (ASCT1) mRNA, complete cds

| | | | | | | | |
|---|---|---|---|---|---|---|---|
| 159C | 292G | 495C | 1305G | 1323T | 1378G | 1773G | 1972A |
| 159C | 292G | 495G | 1305G | 1323C | 1378G | 1773G | 1972A |
| 159C | 292G | 495C | 1305G | 1323C | 1378G | 1773A | 1972A |
| 159C | 292G | 495G | 1305G | 1323C | 1378G | 1773G | 1972T |
| 159C | 292C | 495G | 1305G | 1323C | 1378G | 1773G | 1972A |
| 159G | 292G | 495G | 1305G | 1323C | 1972A | | |
| 159C | 292G | 495G | 1305G | 1323C | 1378G | 1773A | 1972A |
| 159C | 495G | 1305C | 1323C | 1378G | 1773G | 1972A | |
| 159C | 292G | 495G | 1305G | 1323C | 1378A | 1773G | 1972A |
| 159C | 292G | 495C | 1305G | 1323C | 1378G | 1773G | 1972A |
| 159C | 292G | 495G | 1305G | 1323C | 1378A | 1773A | 1972A |
| 159C | 292C | 495G | 1305G | 1323C | 1378A | 1773G | 1972A |
| 159G | 292G | 495G | 1305G | 1323C | 1378A | 1773A | 1972A |
| 159C | 292G | 495C | 1305G | 1323C | 1378A | 1773A | 1972A |
| 159C | 292C | 495G | 1305C | 1323C | 1378G | 1773G | 1972A |

| | | |
|---|---|---|
| 159 | (−25)C > G | 5′ |
| 292 | 109C > G | R37G |
| 495 | 312G > C | Silent |
| 1305 | 1122G > C | Silent |
| 1323 | 1140C > T | Silent |
| 1378 | 1195A > G | 1399V |
| 1773 | 1590G > A | Silent |
| 1972 | 1789A > T | 3′ |

| | | | | |
|---|---|---|---|---|
| X14583 | X14583 | 147240 | GEN-1RJ | Human mRNA for Ig lambda-chain |

| | | |
|---|---|---|
| 131 | 107A > G | K36R |
| 132 | 108G > A | Silent |
| 164 | 140A > C | N47T |
| 255 | 231G > T | Silent |
| 381 | 357A > G | Silent |
| 400 | 376C > G | L126V |
| 412 | 3880 > A | G130S |
| 450 | 426G > A | Silent |
| 522 | 498C > T | Silent |
| 540 | 5160 > C | K172N |
| 553 | 529C > A | P177T |
| 594 | 570A > G | Silent |
| 624 | 600T > C | Silent |
| 639 | 615T > C | Silent |
| 659 | 635G > A | R212K |
| 738 | 714A > C | 3′ |
| 740 | 716A > T | 3′ |
| 752 | 728C > A | 3′ |
| 858 | 834C > G | 3′ |

| | | | | |
|---|---|---|---|---|
| U14650 | U14650 | 602243 | GEN-1Rl | Human platelet-endothelial |

tetraspan antigen 3 mRNA, complete cds

| | | |
|---|---|---|
| 638 | 579A > G | Silent |
| 1048 | 989G > A | 3′ |
| 1171 | 1112T > C | 3′ |
| 1263 | 1204G > C | 3′ |
| 1301 | 1242C > T | 3′ |
| 1351 | 1292T > C | 3′ |
| 1389 | 1330A > T | 3′ |
| 1404 | 1345G > A | 3′ |

| | | | | |
|---|---|---|---|---|
| M14758 | M14758 | 171050 | GEN-1S6 | P glycoprotein 1 |

| | | |
|---|---|---|
| 1623A | 3101G | 3859C |
| 1623A | 3101G | 3859T |
| 1623G | 3101A | |
| 1623G | 3101G | 3859C |
| 1623A | 3101T | 3859C |
| 1623G | 3101T | 3859T |
| 1623G | 3101T | 3859C |
| 1623A | 3101T | 3859T |
| 1623G | 3101G | 3859T |
| 3101T | 3859T | |
| 1623G | 3101A | 3859T |
| 3101G | 3859C | |

| | | |
|---|---|---|
| 1623 | 1199G > A | S400N |
| 3101 | 2677G > A | A893T |
| 3101 | 2677G > T | A893S |
| 3859 | 3435C > T | Silent |
| 4460 | 4036A > G | 3′ |

| | | | | |
|---|---|---|---|---|
| GUSB | M15182 | 253220 | GEN-1TH | Endo-beta-D-glucuronidase |

| | | |
|---|---|---|
| 1766 | 1740T > C | Silent |
| 1972 | 1946C > T | P649L |

| | | | | |
|---|---|---|---|---|
| ADH4 | M15943 | 103740 | GEN-1UM | Human class II alchohol |

dehydrogenase (ADH4) pi subunit mRNA, complete cds

| | | |
|---|---|---|
| 826 | 765G > T | Silent |
| 1389 | 1328T > C | 3′ |

| | | | | |
|---|---|---|---|---|
| HADHB | D16481 | 143450 | GEN-1Y5 | Human mRNA for mitochondrial 3- |

ketoacyl-CoA thiolase beta-subunit of trifunctional protein, complete cds

| | | |
|---|---|---|
| 871 | 825T > C | Silent |
| 1607 | 1561G > C | 3′ |
| 1908 | 1862A > C | 3′ |
| 1911 | 1865A > C | 3′ |

| | | | | |
|---|---|---|---|---|
| U16660 | U16660 | 600696 | GEN-1YD | Human peroxisomal enoyl-CoA |

hydratase-like protein (HPXEL) mRNA, complete cds

| | |
|---|---|
| 149A | 402G |
| 149C | 402G |
| 149C | 402A |
| 149A | 402A |

| | | |
|---|---|---|
| 149 | 122A > C | E41A |
| 402 | 375G > A | Silent |
| 676 | 649G > A | G217R |
| 802 | 775C > G | P259A |
| 877 | 850G > A | D284N |
| 1157 | 1130G > A | 3′ |

| | | | | |
|---|---|---|---|---|
| ELA1 | M16631 | 130120 | GEN-1YI | Human elastase 2 mRNA, complete |
| cds | | | | |

| | | |
|---|---|---|
| 510 | 489G > A | Silent |
| 693 | 672G > A | Silent |

| | | | | |
|---|---|---|---|---|
| X16699 | X16699 | 124075 | GEN-1YJ | Human mRNA for cytochrome P- |
| 450HP | | | | |

| | | |
|---|---|---|
| 1064 | 1064T > G | F355C |

| | | | | |
|---|---|---|---|---|
| X17042 | X17042 | 177040 | GEN-1ZN | Human mRNA for hematopoetic |

proteoglycan core protein

| | | |
|---|---|---|
| 324 | 300C > T | Silent |
| 1021 | 997G > T | 3′ |

| | | | | |
|---|---|---|---|---|
| IGHM | X17115 | 147020 | GEN-1ZX | Human mRNA for IgM heavy chain |

complete sequence

| | | |
|---|---|---|
| 849 | 777T > C | Silent |
| 1102 | 1030A > G | S344G |
| 1107 | 1035G > A | Silent |
| 1175 | 1103T > G | V368G |
| 1212 | 1140C > T | Silent |
| 1561 | 1489C > G | R497G |
| 1692 | 1620G > T | Q540H |
| 1816 | 1744G > C | V582L |
| 2006 | 1934T > A | 3′ |

| | | | | |
|---|---|---|---|---|
| CYP21 | M17252 | 201910 | GEN-201 | Human cytochrome P450c2l mRNA, |

3′ end

| | | | | | |
|---|---|---|---|---|---|
| 745C | 749G | 869C | 1061T | 1066G | 1083A |
| 745C | 869C | 1061T | 1066G | 1083G | |
| 745C | 749C | 569C | 1061T | 1066G | 1083A |
| 745C | 749C | 869C | 1061C | 1066G | |
| 745T | 749C | 869C | 1061C | 1083A | |
| 749C | 569T | 1061T | 1066G | 1053A | |
| 745T | 749C | 869C | 1061T | 1066G | 1083A |
| 745T | 749C | 569T | 1061T | 1066G | 1083A |
| 745C | 869C | 1061T | 1066G | 1083G | |
| 745C | 749C | 869C | 1061C | 1066G | 1053G |
| 745T | 749C | 869C | 1061C | 1066G | 1083G |
| 745T | 749C | 569C | 1061C | 1066A | 1083A |
| 745T | 749C | 869C | 1061C | 1066G | 1053A |

| | | |
|---|---|---|
| 224 | 224G > A | R75H |
| 330 | 330C > T | Silent |
| 745 | 745T > C | 3′ |
| 749 | 749C > G | 3′ |
| 869 | 869C > T | 3′ |
| 1061 | 1061T > C | 3′ |
| 1066 | 1066G > A | 3′ |
| 1083 | 1083A > G | 3′ |

| | | | | |
|---|---|---|---|---|
| D17793 | D17793 | 603966 | GEN-20Q | Human mRNA for KIAA01l9 gene, |

complete cds

| | | |
|---|---|---|
| 66 | 15G > C | Q5H |
| 141 | 90G > A | Silent |
| 363 | 312A > G | Silent |
| 980 | 929G > C | S310T |

| | | | | |
|---|---|---|---|---|
| HSST | U17970 | 600853 | GEN-20V | Human heparan sulfate N- |

deacetylase/N-sulfotransferase mRNA, complete cds

| | | |
|---|---|---|
| 2294 | 2066G > C | G689A |

| | | | | |
|---|---|---|---|---|
| 1317986 | 1317986 | 300036 | GEN-20X | Human GABA/noradrenaline |

transporter mRNA, complete cds

| | | |
|---|---|---|
| 1161 | 1132G > A | V378M |
| 1670 | 1641C > T | Silent |
| 2034 | 2005G > A | V669M |
| 2088 | 2059C > T | R687C |
| 2150 | 2121C > T | Silent |
| 2231 | 2202A > G | 3′ |

| | | | | |
|---|---|---|---|---|
| AHR | L19872 | 600253 | GEN-22N | Human AH-receptor mRNA, complete |
| cds | | | | |

| | | |
|---|---|---|
| 4722 | 4347G > A | 3′ |

| | | | | |
|---|---|---|---|---|
| U19977 | U19977 | 600688 | GEN-22Q | Human preprocarboxypeptidase A2 |

(proCPA2) mRNA, complete cds

| | | |
|---|---|---|
| 631 | 627C > T | Silent |

| | | | | |
|---|---|---|---|---|
| AF019386 | AF019386 | 603244 | GEN-231 | Homo sapiens heparan sulfate 3- |

O-sulfotransferase-1 precursor (30ST1) mRNA, complete cds

| | | |
|---|---|---|
| 79 | (−40)C > G | 5′ |

| | | | | |
|---|---|---|---|---|
| U20157 | U20157 | 601690 | GEN-234 | Human platelet-activating factor |

acetylhydrolase mRNA, complete cds

| | | |
|---|---|---|
| 1297 | 1136T > C | V379A |

| | | | | |
|---|---|---|---|---|
| M20681 | M20681 | 138170 | GEN-23O | Human glucose transporter-like |

protein-III (GLUT3), complete cds

| | | |
|---|---|---|
| 1550 | 1308C > T | Silent |
| 3179 | 2937T > C | 3′ |
| 3238 | 2996C > T | 3′ |
| 3356 | 3114T > C | 3′ |
| 3378 | 3136T > C | 3′ |
| 3524 | 3282C > A | 3′ |
| 3572 | 3330G > T | 3′ |

| | | | | |
|---|---|---|---|---|
| SLC2A4 | M20747 | 138190 | GEN-23Q | Human insulin-responsive glucose |

transporter (GLUT4) mRNA, complete cds

| | | |
|---|---|---|
| 535C | 1182G | 1218T |
| 535C | 1182G | 1218C |
| 535T | 1182G | 1218C |
| 535C | 1182A | 1218C |

| | | |
|---|---|---|
| 535 | 390C > T | Silent |
| 1182 | 1037G > A | R346Q |
| 1218 | 1073C > T | A358V |

| | | | | |
|---|---|---|---|---|
| SOAT | L21934 | 102642 | GEN-25C | Human acyl coenzyme |

A:cholesterol acyltransferase mRNA, complete cds

| | | | | | | | | | | | |
|---|---|---|---|---|---|---|---|---|---|---|---|
| 92T | 93T | 121T | 379A | 490C | 814C | 2365C | 2821C | 2973A | 3083G | | |
| 92T | 93T | 121T | 379G | 490C | 676T | 814C | 1993T | 2170C | 2365C | 2821C | 2973A |
| 3083G | | | | | | | | | | | |
| 92T | 93C | 121T | 379G | 490C | 676T | 814C | 1993C | 2365C | 2821C | | |
| 92T | 121C | 379G | 490C | 676G | 814C | 1993C | 2170C | 2365C | 2821C | 2973A | 3083G |
| 92T | 93T | 121T | 379G | 490C | 676T | 814C | 1993C | 2170C | 2821G | 3083G | |
| 92T | 93T | 121T | 379G | 490C | 676G | 814C | 1993C | 2170C | 2365C | 2821C | 2973A |
| 3083G | | | | | | | | | | | |
| 92T | 93T | 121T | 379G | 490C | 676T | 814C | 1993C | 2170C | 2365C | 2821C | 2973A |
| 3083G | | | | | | | | | | | |
| 92T | 93T | 121T | 490G | 676G | 814C | 1993C | 2170C | 2365C | 2821C | 2973A | 3083G |
| 92T | 93T | 121T | 379G | 490C | 676T | 814C | 1993C | 2170T | 2365C | 2821C | 2973A |
| 3083G | | | | | | | | | | | |
| 92C | 93T | 121T | 379G | 490C | 676G | 814C | 1993C | 2170C | 2821C | 2973A | 3083G |
| 92T 121C | 379G | 490G | 676G | 814C | 1993T | 2170C | 2365C | 2821C | 2973A | 3083G | |
| 92C | 93T | 121T | 379G | 490C | 814C | 1993T | 2170C | 2365C | 2821C | 2973A | 3083G |
| 92T | 93T | 121T | 379G | 676G | 1993C | 2821G | 2973A | 3083G | | | |
| 92T | 93T | 121T | 379G | 490C | 814C | 1993C | 2365C | 2821C | 2973A | 3083T | |
| 92T | 93T | 121T | 379G | 490C | 676T | 1993T | 2170C | 2365T | 2821C | 2973A | 3083G |
| 92T | 93T | 121T | 379G | 490C | 676T | 814C | 1993C | 2365T | 2821C | | |
| 92T | 93T | 121T | 379G | 676T | 1993C | 2821G | 2973A | 3083G | | | |
| 92T | 93C | 121C | 379G | 490C | 676G | 814C | 1993T | 2170C | 2365C | 2821C | 2973A |
| 3083G | | | | | | | | | | | |
| 92T | 93T | 121T | 379A | 490G | 676G | 814C | 1993C | 2170C | 2365C | 2821C | 2973A |
| 3083G | | | | | | | | | | | |
| 490C | 676G | 814C | 1993C | 2365C | 2821C | | | | | | |
| 92T | 93T | 121T | 379G | 490C | 676G | 814C | 1993T | 2170C | 2365C | 2821C | 2973A |
| 3083G | | | | | | | | | | | |
| 490C | 676G | 814C | 1993C | 2170C | 2365C | 2821C | 2973A | 3083G | | | |
| 92C | 93T | 121T | 379G | 490C | 676G | 814C | 1993T | 2170C | 2365C | 2821C | 2973A |
| 3083G | | | | | | | | | | | |
| 92T | 93T | 121T | 379G | 490C | 676G | 814C | 1993C | 2170C | 2365T | 2821C | 2973G |
| 3083G | | | | | | | | | | | |
| 92T | 93T | 121T | 379G | 676T | 1993C | 2170C | 2365C | 2821C | 2973A | 3083G | |
| 92T | 93T | 121T | 379A | 490C | 676G | 814C | 1993C | 2170T | 2365C | 2821C | 2973A |
| 3083G | | | | | | | | | | | |
| 92T | 93T | 121T | 379G | 490C | 676T | 814C | 1993C | 2170T | 2365C | 2821C | 2973A |
| 3083T | | | | | | | | | | | |
| 92T | 93C | 121T | 379G | 490C | 676T | 814C | 1993C | 2170T | 2365T | 2821G | 2973A |
| 3083G | | | | | | | | | | | |
| 92T | 93T | 121T | 379G | 490C | 676T | 814T | 1993T | 2170C | 2365T | 2821C | 2973A |
| 3083G | | | | | | | | | | | |
| 92T | 93T | 121T | 379G | 490C | 676T | 814C | 1993C | 2170C | 2365T | 2821G | 2973G |
| 3083G | | | | | | | | | | | |
| 92T | 93T | 121T | 379G | 490C | 676T | 814C | 1993C | 2170T | 2365T | 2821C | |
| 92T | 93T | 121T | 379G | 490G | 676G | 814C | 1993C | 2170C | 2365C | 2821C | 2973A |
| 3083G | | | | | | | | | | | |
| 92T | 93T | 121T | 379G | 490G | 676G | 814C | 1993C | 2365T | 2821G | 2973G | 3083G |
| 92T | 93C | 121T | 379G | 490C | 676T | 814C | 1993C | 2170T | 2365C | 2821C | |
| 92C | 93C | 121T | 379G | 490C | 676T | 814C | 1993C | 2365T | 2821C | 2973A | 3083G |
| 676G | 814C | 1993C | 2365T | 2821G | | | | | | | |
| 92T | 93T | 121T | 379G | 490C | 676G | 814C | 1993C | 2170C | 2365T | 2821C | 2973A |
| 3083G | | | | | | | | | | | |

| | | |
|---|---|---|
| 92 | (−1305)T > C | 5′ |
| 93 | (−1304)T > C | 5′ |
| 121 | (−1276)T > C | 5′ |
| 379 | (−1018)A > A | 5′ |
| 490 | (−907)G > G | 5′ |
| 676 | (−721)G > G | 5′ |
| 814 | (−583)T > T | 5′ |
| 1993 | 597C > T | Silent |
| 2170 | 774C > T | Silent |
| 2365 | 969C > T | Silent |
| 2821 | 1425G > C | Silent |
| 2973 | 1577G > A | R526Q |
| 3083 | 1687G > T | 3′ |
| 3537 | 2141T > C | 3′ |

| | | | | |
|---|---|---|---|---|
| M22324 | M22324 | 151530 | GEN-25R | Human aminopeptidase N/CD13 mRNA |

encoding aminopeptidase N, complete cds

| | | |
|---|---|---|
| 1052 | 932C > T | A311V |
| 2168 | 2048C > G | T683S |
| 2375 | 2255G > A | S752N |
| 2505 | 2385C > T | Silent |
| 3053 | 2933G > C | 3′ |
| 3299 | 3179A > G | 3′ |
| 3405 | 3285C > T | 3′ |

| | | | | |
|---|---|---|---|---|
| PLA2G2A | M22430 | 172411 | GEN-25V | Human RASF-A PLA2 mRNA, complete |
| cds | | | | |

| | | |
|---|---|---|
| 116 | (−20)G > T | 5′ |
| 267 | 132C > T | Silent |

| | | | | |
|---|---|---|---|---|
| AF026947 | AF026947 | 603418 | GEN-261 | Homo sapiens aflatoxin aldehyde |

reductase AFAR mRNA, complete cds

| | | |
|---|---|---|
| 1013 | 936T > C | Silent |
| 1078 | 1001A > G | 3′ |

| | | | | |
|---|---|---|---|---|
| PRSS1 | M22612 | 276000 | GEN-26A | Human pancreatic trypsin 1 |

(TRY1) mRNA, complete cds

| | | |
|---|---|---|
| 34 | 28G > T | V10L |
| 61 | 55G > A | D19N |
| 97 | 91G > A | E31K |
| 198 | 192C > T | Silent |
| 412 | 406G > T | G136C |
| 492 | 486T > C | Silent |
| 711 | 705C > T | Silent |
| 744 | 738T > C | Silent |

| | | | | |
|---|---|---|---|---|
| TAP2 | Z22935 | 170261 | GEN-26P | H. sapiens TAP2B mRNA, complete |
| CDS | | | | |

| | |
|---|---|
| 1186G | 1336T |
| 1186T | 1336C |
| 1186G | 1336C |

| | | |
|---|---|---|
| 1163 | 1135G > A | V379I |
| 1186 | 1158G > T | Silent |
| 1336 | 1308T > C | Silent |
| 1840 | 1812G > A | Silent |
| 2021 | 1993G > A | A665T |
| 2087 | 2059C > T | Frame |
| 2119 | 2091T > G | Silent |

| | | | | |
|---|---|---|---|---|
| HRH1 | AF026261 | 600167 | GEN-26W | Histamine receptor H1 |

| | | |
|---|---|---|
| 1068 | 1068A > G | Silent |

| | | | | |
|---|---|---|---|---|
| HLA-DMB | Z23139 | 142856 | GEN-277 | H. sapiens RING7 mLRNA for HLA |

class II alpha chain-like product

| | | |
|---|---|---|
| 380 | 212G > A | S71N |
| 1125 | 957C > T | 3′ |

| | | | | |
|---|---|---|---|---|
| CYP51 | U23942 | 601637 | GEN-27K | Human lanosterol 14-demethylase |

cytochrome P450 (CYP51) mRNA, complete cds

| | | |
|---|---|---|
| 766 | 644G > A | C215Y |
| 894 | 772C > T | R258C |
| 912 | 790C > T | R264W |
| 1476 | 1354C > T | R452C |
| 1616 | 1494G > A | Silent |
| 1836 | 1714C > A | 3′ |
| 2283 | 2161G > T | 3′ |
| 2445 | 2323T > C | 3′ |
| 2507 | 2385G > A | 3′ |
| 2556 | 2434T > A | 3′ |
| 2665 | 2543G > A | 3′ |

| | | | | |
|---|---|---|---|---|
| AF027302 | AF027302 | 603429 | GEN-27T | Homo sapiens TNF-alpha |

stimulated ABC protein (ABC50) mPNA, complete cds

| | | |
|---|---|---|
| 3075 | 2981T > C | 3′ |

| | | | | |
|---|---|---|---|---|
| SLC6A3 | L24178 | 126455 | GEN-283 | Homo sapiens dopamine |

transporter mRNA, complete cds

| | | |
|---|---|---|
| 169T | 181C | 244C |
| 169G | 181T | 244C |
| 169G | 181C | 244C |
| 169T | 181C | 244T |

| | | |
|---|---|---|
| 169 | 150G > T | Silent |
| 181 | 162C > T | Silent |
| 244 | 225C > T | Silent |
| 1917 | 1898C > T | 3′ |

| | | | | |
|---|---|---|---|---|
| ADH2 | M24317 | 103720 | GEN-28A | Human class I alcohol |

dehydrogenase (ADH2) beta-1 subunit mRNA, complete cds

| | | |
|---|---|---|
| 817 | 787G > A | V263M |

| | | | | |
|---|---|---|---|---|
| SLC5A1 | M24847 | 182380 | GEN-28S | Human Na+/glucose cotransporter |

1 mRNA, complete cds

| | | |
|---|---|---|
| 2226 | 2216C > T | 3′ |

| | | | | |
|---|---|---|---|---|
| U25147 | U25147 | 190315 | GEN-294 | Human citrate transporter |

protein mRNA, nuclear gene encoding mitochondrial protein, complete cds

| | | |
|---|---|---|
| 353 | 279T > C | Silent |

| | | | | |
|---|---|---|---|---|
| L25259 | L25259 | 601020 | GEN-298 | Human CTLA4 counter-receptor |

(B7-2) mRNA, complete cds

| | | |
|---|---|---|
| 1034 | 928G > A | A310T |

| | | | | |
|---|---|---|---|---|
| EPHX1 | L25878 | 132810 | GEN-29Z | Homo sapiens p33/HEH epoxide |

hydrolase (EPHX) mRNA, complete cds

| | | |
|---|---|---|
| 460 | 337T > C | Y113H |
| 480 | 357A > G | Silent |
| 539 | 416A > G | H139R |
| 1194 | 1071C > T | Silent |

| | | | | |
|---|---|---|---|---|
| ATM | U26455 | 208900 | GEN-2AT | Human phosphatidylinositol 3- |

kinase homolog (ATM) mRNA, complete cds

| | | | | | | | |
|---|---|---|---|---|---|---|---|
| 1772G | 2409C | 2450G | 2652G | 5430A | 5622C | | |
| 793T | 1772G | 2409T | 2450G | 2652G | 3210T | 5430A | 5622C |
| 793C | 1772G | 2409T | 2450G | 2652G | 3210T | 5430A | 5622C |
| 793C | 1772A | 2409T | 2450G | 2652G | 3210T | 5430A | 5622C |
| 793C | 1772G | 2409T | 2450A | 3210T | | | |
| 793T | 1772G | 2409C | 2450G | 2652G | 3210C | 5430A | 5622C |
| 793C | 1772G | 2409T | 2450A | 2652C | 3210T | 5430G | 5622T |

| | | |
|---|---|---|
| 793 | 534C > T | Silent |
| 1772 | 1513G > A | DS0SN |
| 2409 | 2150T > C | I717T |
| 2450 | 2191G > A | V731I |
| 2652 | 2393G > C | 8798T |
| 3210 | 2951T > C | L984P |
| 5222 | 4963G > A | D1655N |
| 5308 | 5049G > A | Silent |
| 5430 | 5171A > G | 3′ |
| 5622 | 5363C > T | 3′ |
| 5626 | 5367T > G | 3′ |

| | | | | |
|---|---|---|---|---|
| D26579 | D26579 | 602267 | GEN-2B1 | Human mRNA for transmembrane |

protein, complete cds

| | | |
|---|---|---|
| 709 | 700G > A | D234N |
| 909 | 900T > C | Silent |
| 999 | 990C > T | Silent |
| 1104 | 1095A > G | Silent |

| Z26649 | | Z26649 | | 600230 | | GEN-2B5 | | Phospholipase C beta-3 | | | |
|---|---|---|---|---|---|---|---|---|---|---|---|
| | 437C | 466G | 952G | 1342A | 1578G | 1624C | 1794G | 3168G | 3466G | 3673G | 3718G |
| | 437C | 466G | 952G | 1342A | 1578A | 1624C | 1794G | 3168G | 3466G | 3673G | 3718G |
| | 437C | 466G | 952G | 1578G | 1624C | 1794A | 3168G | 3466G | 3673G | 3718G | |
| | 437C | 466G | 952A | 1342A | 1578G | 1624T | 1794G | 3168G | 3466G | 3673G | 3718G |
| | 437C | 466G | 952G | 1342G | 1578G | 1624C | 1794G | 3168G | 3466G | 3673A | |
| | 437T | 466G | 952G | 1342G | 3168G | 3466G | 3673G | 3718A | | | |
| | 437C | 466G | 952A | 1342G | 1578G | 1794G | 3168G | 3466G | 3718G | | |
| | 437C | 466G | 952G | 1342G | 1578G | 1624C | 1794G | 3168T | 3466G | 3673G | |
| | 437C | 466A | 952G | 1342G | 1578G | 1624C | 1794G | 3168G | 3466G | 3673G | 3718G |
| | 437C | 466G | 952G | 1342G | 1578G | 1624C | 1794G | 3168G | 3466G | 3673G | 3718A |
| | 437C | 466G | 952G | 1342G | 1578G | 1624C | 1794G | 3168G | 3466A | 3718G | |
| | 437C | 466G | 952G | 1342G | 1578G | 1624C | 1794G | 3168G | 3466G | 3673G | 3718G |
| | 437C | 466G | 952G | 1342G | 1578G | 1624C | 1794G | 3168G | 3466G | 3673A | 3718A |
| | 437C | 466G | 952G | 1342G | 3168G | 3466G | 3673G | 3718A | | | |
| | 437C | 466G | 952G | 1342G | 1578G | 1624C | 1794G | 3168G | 3466G | 3673A | 3718G |
| | 437C | 466G | 952A | 1342G | 1578G | 1624T | 1794G | 3168G | 3466G | 3673A | 3718G |
| | 437C | 466G | 952A | 1342A | 1578G | 1624T | 1794G | 3168G | 3466G | 3673A | 3718G |
| | 437C | 466G | 952G | 1342A | 1578G | 1624C | 1794A | 3168G | 3466G | 3673G | 3718G |
| | 437T | 466G | 952G | 1342G | 3168G | 3466G | 3673G | 3718A | | | |
| | 437C | 466G | 952G | 1342A | 1578G | 1624C | 1794G | 3168G | 3466G | 3673A | 3718G |
| | 466G | 952A | 1342A | 1578G | 1624T | 1794G | 3168G | 3673G | 3718G | | |
| | 466G | 952G | 1342G | 1578G | 1624C | 1794G | 3168G | 3673G | 3718G | | |
| | 437C | 466G | 952G | 1342G | 1578G | 1624C | 1794G | 3168T | 3466G | 3673G | 3718A |

| | | |
|---|---|---|
| 437 | 437C > T | 3′ |
| 466 | 466G > A | 3′ |
| 952 | 952G > A | 3′ |
| 1342 | 1342G > A | 3′ |
| 1578 | 1578G > A | 3′ |
| 1624 | 1624C > T | 3′ |
| 1794 | 1794G > A | 3′ |
| 2664 | 2664C > T | 3′ |
| 3168 | 3168G > T | 3′ |
| 3466 | 3466G > A | 3′ |
| 3673 | 3673G > A | 3′ |
| 3718 | 3718G > A | 3′ |

| | | | | |
|---|---|---|---|---|
| PRSS2 | M27602 | 601564 | GEN-2C7 | Human pancreatic trypsinogen |

(TRY2) mRNA, complete cds

| | | |
|---|---|---|
| 29 | 23C > T | T8I |
| 34 | 28G > T | V10F |
| 61 | 55G > A | D19N |
| 97 | 91G > A | E31K |
| 198 | 192C > T | Silent |
| 276 | 270G > A | Silent |

| | | | | |
|---|---|---|---|---|
| U27699 | U27699 | 603080 | GEN-2C9 | Human pephBGT-1 betaine-GABA |

transporter mRNA, complete cds

| | | | | | | | | |
|---|---|---|---|---|---|---|---|---|
| 1033C | 2498G | 2643G | 2655C | 2681G | 2775A | 2947C | 3120T | 3266A |
| 1033C | 2498G | 2643G | 2655C | 2681G | 2775G | 2947T | 3120C | 3266G |
| 1033T | 2498G | 2643G | 2655C | 2681G | 2775G | 2947T | 3120C | 3266A |
| 1033T | 2498A | 2655C | 2681G | 2775A | 2947C | 3120T | 3266A | |
| 1033C | 2498A | 2655C | 2681G | 2775A | 2947C | 3120T | 3266A | |
| 1033T | 2498G | 2643G | 2655C | 2681G | 2775A | 2947C | 3120T | 3266A |
| 1033C | 2498G | 2643G | 2655C | 2681G | 2947T | 3120T | 3266A | |
| 2498G | 2643G | 2655T | 2681G | 2947C | 3266A | | | |
| 1033T | 2498G | 2643G | 2655C | 2681G | 2775G | 2947C | 3266A | |
| 2498G | 2643G | 2655C | 2681A | 2775A | 2947C | 3120T | 3266A | |
| 1033T | 2498G | 2643G | 2655C | 2681G | 2775G | 2947T | 3120T | 3266A |
| 1033C | 2498G | 2643G | 2655C | 2681G | 2775G | 2947C | 3266A | |
| 1033T | 2498G | 2643G | 2655C | 2681G | 2775A | 3120C | 3266A | |
| 1033C | 2498G | 2643G | 2655C | 2681G | 2775G | 2947T | 3120C | 3266A |
| 1033T | 2498G | 2643G | 2655C | 2681G | 2775G | 2947C | 3120T | 3266A |
| 1033C | 2643G | 2655C | 2681G | 2775A | 2947C | 3120T | 3266A | |
| 1033C | 2498A | 2643A | 2655C | 2681G | 2775A | 2947C | 3120T | 3266A |
| 1033C | 2498G | 2643G | 2655C | 2681G | 2775G | 2947T | 3120T | 3266A |
| 1033C | 2498G | 2643G | 2655C | 2681G | 2775G | 2947C | 3120C | 3266A |
| 1033C | 2498G | 2643G | 2655C | 2681G | 2775G | 2947C | 3120T | 3266A |
| 1033C | 2498A | 2643G | 2655C | 2681G | 2775G | 2947C | 3120T | 3266A |
| 1033C | 2498G | 2947T | 3120C | 3266A | | | | |
| 1033T | 2498G | 2643G | 2655C | 2681A | 2775A | 2947C | 3120T | 3266A |
| 1033T | 2498A | 2643A | 2655C | 2681G | 2775A | 2947C | 3120T | 3266A |
| 1033T | 2498G | 2643G | 2655C | 2681G | 2775A | 2947T | 3120C | 3266A |
| 1033T | 2643G | 2655C | 2681G | 2775A | 2947C | 3120T | 3266A | |
| 1033T | 2498G | 2643G | 2655T | 2681G | 2775G | 2947C | 3120C | 3266A |

| | | |
|---|---|---|
| 1033 | 447T > C | Silent |
| 2498 | 1912G > A | 3′ |
| 2643 | 2057G > A | 3′ |
| 2655 | 2069C > T | 3′ |
| 2681 | 2095G > A | 3′ |
| 2775 | 2189G > A | 3′ |
| 2841 | 2255C > T | 3′ |
| 2947 | 2361T > C | 3′ |
| 3120 | 2534C > T | 3′ |
| 3266 | 2680A > G | 3′ |

| | | | | |
|---|---|---|---|---|
| CFTR | M28668 | 602421 | GEN-2DF | Human cystic fibrosis mRNA, |

encoding a presumed transmembrane conductance regulator (CFTR)

| | | |
|---|---|---|
| 2729 | 2597G > A | C866Y |
| 5826 | 5694T > C | 3′ |

| | | | | |
|---|---|---|---|---|
| ADHS | M29872 | 103710 | GEN-2EU | Human alcohol dehydrogenase |

class III (ADH5) mRNA, complete cds

| | | |
|---|---|---|
| 1029 | 1025G > A | S342N |
| 1375 | 1371T > C | 3′ |

| | | | | |
|---|---|---|---|---|
| AF028738 | AF028738 | 602631 | GEN-2F6 | Homo sapiens imprinted multi- |

membrane spanning polyspecific transporter-related protein (IMPT1) mRNA,
complete cds

| | | |
|---|---|---|
| 34 | (−209)A > C | 5′ |
| 210 | (−33)G > A | 5′ |
| 229 | (−14)A > G | 5′ |
| 375 | 133T > G | F45V |
| 875 | 633A > C | E211D |
| 881 | 639A > G | Silent |
| 883 | 641G > C | G214A |
| 919 | 677A > G | K226R |
| 927 | 685T > C | Silent |
| 935 | 693A > G | Silent |
| 1004 | 762A > G | Silent |
| 1017 | 775A > C | K259Q |
| 1106 | 864A > G | Silent |
| 1119 | 877G > C | G293R |
| 1124 | 882A > C | Silent |
| 1166 | 924G > C | W308C |

| | | | | |
|---|---|---|---|---|
| AF034374 | AF034374 | None | GEN-2GC | Homo sapiens molybdenum cofactor |

biosynthesis protein A and molybdenum cofactor biosynthesis protein C mRNA,
complete cds

| | | |
|---|---|---|
| 2628 | 1435C > A | 3′ |
| 2677 | 1484C > G | 3′ |
| 2742 | 1549A > T | 3′ |

| | | | | |
|---|---|---|---|---|
| L32179 | L32179 | 600338 | GEN-2IW | Human arylacetamide deacetylase |

mRNA, complete cds

| | | |
|---|---|---|
| 1366 | 1281G > A | 3′ |

| | | | | |
|---|---|---|---|---|
| NRAMP1 | L32185 | 600266 | GEN-2IY | Homo sapiens integral membrane |

protein (NRAMP1) mRNA, complete cds

| | | |
|---|---|---|
| 1399 | 1323C > T | Silent |

| | | | | |
|---|---|---|---|---|
| ARSB | M32373 | 253200 | GEN-2J0 | Human arylsulfatase B (ASB) |

mRNA, complete cds

| | | |
|---|---|---|
| 1631 | 1072G > A | V358M |

| | | | | |
|---|---|---|---|---|
| U32989 | U32989 | 191070 | GEN-2JH | Human tryptophan oxygenase (TDO) |

mRNA, complete cds

| | | |
|---|---|---|
| 991 | 927G > A | Silent |

| | | | | |
|---|---|---|---|---|
| M33195 | M33195 | 147139 | GEN-2JR | Human Fc-epsilon-receptor gamma- |

chain mRNA, complete cds

| | | |
|---|---|---|
| 446 | 421T > G | 3′ |
| 489 | 464T > C | 3′ |

| | | | | |
|---|---|---|---|---|
| HLA-DQB1 | M33907 | 142857 | GEN-2KB | Human MHC class II HLA-DQB1 |

mRNA, complete cds

| | | |
|---|---|---|
| 561 | 516T > C | Silent |
| 641 | 596G > A | R199H |
| 648 | 603C > T | Silent |
| 695 | 650T > C | I217T |
| 771 | 726G > C | Silent |
| 780 | 735C > T | Silent |

| | | | | |
|---|---|---|---|---|
| AF037335 | AF037335 | 603263 | GEN-2KJ | Homo sapiens carbonic anhydrase |

precursor (CA 12) mRNA, complete cds

| | | |
|---|---|---|
| 1551 | 1436G > T | 3′ |
| 2442 | 2327C > T | 3′ |

| | | | | |
|---|---|---|---|---|
| GSS | U34683 | 601002 | GEN-2LF | Human glutathione synthetase |

mRNA, complete cds

| | |
|---|---|
| 1467G | 1482T |
| 1467A | 1482C |
| 1467G | 1482C |

| | | |
|---|---|---|
| 364 | 324G > A | Silent |
| 1467 | 1427G > A | 3′ |
| 1482 | 1442C > T | 3′ |

| | | | | |
|---|---|---|---|---|
| U35735 | U35735 | 111000 | GEN-2MN | Human RACH1 (RACH1) mRNA, |

complete cds

| | | |
|---|---|---|
| 1006 | 838A > G | N280D |
| 2619 | 2451T > C | 3′ |
| 2706 | 2538T > C | 3′ |

| | | | | |
|---|---|---|---|---|
| LIG1 | M36067 | 126391 | GEN-2MS | Human DNA ligase I mRNA, |

complete cds

| | | |
|---|---|---|
| 2526 | 2406T > C | Silent |

| | | | | |
|---|---|---|---|---|
| M36712 | M36712 | 186730 | GEN-2NC | Human T lymphocyte surface |

glycoprotein (CD8-beta) mPNA, complete cds

| | | |
|---|---|---|
| 986G | 1004A | 1047A |
| 986G | 1004G | 1047G |
| 986G | 1004A | 1047G |
| 986A | 1004G | 1047G |

| | | |
|---|---|---|
| 986 | 941G > A | 3′ |
| 1004 | 959A > G | 3′ |
| 1046 | 1001C > A | 3′ |
| 1047 | 1002G > A | 3′ |
| 1281 | 1236T > C | 3′ |
| 1326 | 1281C > A | 3′ |

| | | | | |
|---|---|---|---|---|
| U37143 | U37143 | 601258 | GEN-2NS | Human cytochrome P450 |

monooxygenase CYP2J2 mRNA, complete cds

| | | |
|---|---|---|
| 338 | 333G > C | Silent |
| 1545 | 1540C > T | 3′ |

| | | | | |
|---|---|---|---|---|
| NRAMP2 | 137347 | 600523 | GEN-2O6 | Human integral membrane protein |

(Nramp2) mRNA, partial

| | | |
|---|---|---|
| 1092 | 1083C > T | Silent |

| | | | | |
|---|---|---|---|---|
| GSTT2 | L38503 | 600437 | GEN-2PC | Homo sapiens glutathione S- |

transferase theta 2 (GSTT2) mRNA, complete cds

| | | |
|---|---|---|
| 203 | 203C > T | S68L |
| 543 | 543C > T | Silent |

| | | | | |
|---|---|---|---|---|
| ALCAM | L38608 | 601662 | GEN-2PJ | Homo sapiens CD6 ligand (ALCAM) |

mRNA, complete cds

| | | |
|---|---|---|
| 1041C | 1344T | 1401A |
| 1041C | 1344C | 1401A |
| 1041C | 1344T | 1401G |
| 1041T | 1344T | 1401A |
| 1041T | 1344T | 1401G |

| | | |
|---|---|---|
| 1041 | 978C > T | Silent |
| 1344 | 1281T > C | Silent |
| 1401 | 1338G > A | Silent |

| | | | | |
|---|---|---|---|---|
| L38928 | L38928 | 604197 | GEN-2PT | Homo sapiens 5,10- |

methenyltetrahydrofolate synthetase mRNA, complete cds

| | | |
|---|---|---|
| 617 | 604A > G | T202A |

| | | | | |
|---|---|---|---|---|
| AF038007 | AF038007 | 602397 | GEN-2QG | Homo sapiens P-type ATPase FIC1 |

mRNA, partial cds

| | | | |
|---|---|---|---|
| 152C | 829C | 2873G | 3495C |
| 152A | 829C | 2873A | 3495C |
| 152A | 3495T | | |
| 152A | 829C | 2873G | 3495C |
| 152A | 829A | 2873G | 3495C |
| 152A | 2873G | | |

| | | |
|---|---|---|
| 152 | 152A > C | N51T |
| 829 | 829C > A | Silent |
| 2873 | 2873G > A | R958Q |
| 3495 | 3495C > T | Silent |

| | | | | |
|---|---|---|---|---|
| AF038175 | AF038175 | 603076 | GEN-2QM | Homo sapiens clone 23819 white |

protein homolog mRNA, partial cds

| | | |
|---|---|---|
| 1100 | 1100G > A | 3′ |

| | | | | |
|---|---|---|---|---|
| U40347 | U40347 | 600950 | GEN-2RK | Human serotonin N- |

acetyltransferase mRNA, complete cds

| | | |
|---|---|---|
| 382 | 148G > A | E50K |

| | | | | |
|---|---|---|---|---|
| IDS | L40586 | 309900 | GEN-2SB | Homo sapiens iduronate-2- |

sulphatase (IDS) mRNA, complete cds

| | | |
|---|---|---|
| 565 | 438C > T | Silent |

| | | | | |
|---|---|---|---|---|
| L40992 | L40992 | 600211 | GEN-2SO | Homo sapiens (clone PEBP2aA1) |

core-binding factor, runt domain, alpha subunit 1 (CBFA1) mRNA, 3′ end of cds

| | | |
|---|---|---|
| 265 | 265G > A | V89I |

| | | | | |
|---|---|---|---|---|
| SCYA11 | U46573 | 601156 | GEN-2WZ | Human eotaxin precursor mRNA, |

complete cds

| | | |
|---|---|---|
| 120 | 67G > A | A23T |
| 554 | 501T > C | 3′ |

| | | | | |
|---|---|---|---|---|
| ALDH10 | L47162 | 270200 | GEN-2XI | Human fatty aldehyde |

dehydrogenase (FALDH) mRNA, complete cds

| | | |
|---|---|---|
| 1609 | 1446A > T | Silent |

| | | | | |
|---|---|---|---|---|
| Z47553 | Z47553 | 603957 | GEN-2XN | H. sapiens mRNA for flavin |

containing monooxygenase 5 (FMO5)

| | | |
|---|---|---|
| 1092 | 1011A > G | Silent |

| | | | | |
|---|---|---|---|---|
| L48513 | L48513 | 602447 | GEN-2YD | Homo sapiens paraoxonase 2 |

(PON2) mRNA, complete cds

| | | |
|---|---|---|
| 460 | 443C > G | A148G |
| 598 | 581G > A | G194H |
| 949 | 932G > C | C311S |

| | | | | |
|---|---|---|---|---|
| D49737 | D49737 | 602413 | GEN-2Z7 | Homo sapiens mRNA for cytochrome |

b large subunit of complex II, complete cds

| | | |
|---|---|---|
| 908 | 784G > A | 3′ |

| | | | | |
|---|---|---|---|---|
| U50040 | U50040 | 601582 | GEN-2ZR | Human signaling inositol |

polyphosphate 5 phosphatase SIP-110 mRNA, complete cds

| | | |
|---|---|---|
| 196 | 180A > G | Silent |
| 418 | 402C > G | Silent |
| 2613 | 2597C > A | P866H |
| 2638 | 2622G > A | Silent |
| 2882 | 2866C > T | H956Y |
| 3193 | 3177C > T | 3′ |
| 3222 | 3206C > T | 3′ |
| 3863 | 3847G > A | 3′ |

| | | | | |
|---|---|---|---|---|
| U51478 | U51478 | 601867 | GEN-31Z | Human sodium/potassium- |

transporting ATPase beta-3 subunit mRNA, complete cds

| | | |
|---|---|---|
| 1099 | 1071G > C | 3′ |
| 1121 | 1093T > C | 3′ |
| 1133 | 1105G > T | 3′ |

| | | | | |
|---|---|---|---|---|
| AF055025 | AF055025 | 300095 | GEN-32U | Homo sapiens clone 24776 mRNA |
| sequence | | | | |

| | | |
|---|---|---|
| 784 | 784A > G | 3′ |
| 2021 | 2021A > T | 3′ |

| | | | | |
|---|---|---|---|---|
| X52079 | X52079 | 602272 | GEN-33B | H. sapiens transcription factor |

(ITF-2) mRNA, 3′ end

| | | |
|---|---|---|
| 979 | 979T > G | S327A |
| 1794 | 1794G > A | Silent |

| | | | | |
|---|---|---|---|---|
| CTH | S52028 | 219500 | GEN-33F | cystathionine gamma-lyase {clone |

HCL-1} [human, liver, mRNA, 1194 nt]

| | | |
|---|---|---|
| 1109 | 1076T > G | I359S |

| | | | | |
|---|---|---|---|---|
| X52125 | X52125 | 189990 | GEN-33J | Human alternatively spliced c- |
| myb mRNA (clone = pMbm − 1) | | | | |

| | | |
|---|---|---|
| 1727C | 2096G | 2451G |
| 1727T | 2096G | 2451G |
| 1727T | 2096A | 2451G |
| 1727T | 2096G | 2451C |

| | | |
|---|---|---|
| 1727 | 1530T > C | Silent |
| 2096 | 1899G > A | Silent |
| 2380 | 2183\hat{ }2184insA | 3′ |
| 2451 | 2254G > C | 3′ |

| | | | | |
|---|---|---|---|---|
| PDHA1 | X52709 | 312170 | GEN-33Y | Human mRNA for brain pyruvate |

dehydrogenase (EC 1.2.4.1)

| | | |
|---|---|---|
| 849 | 795A > G | Silent |
| 1337 | 1283C > T | 3′ |
| 1416 | 1362G > A | 3′ |

| | | | | |
|---|---|---|---|---|
| CD22 | X52785 | 107266 | GEN-33Z | H. sapiens CD22 mRNA |

| | | |
|---|---|---|
| 1357 | 1323C > T | Silent |
| 1531 | 1497C > T | Silent |

| | | | | |
|---|---|---|---|---|
| U53347 | U53347 | 109190 | GEN-34A | Human neutral amino acid |

transporter B mRNA, complete cds

| | | | | | | | | | | | |
|---|---|---|---|---|---|---|---|---|---|---|---|
| 272C | 281T | 337T | 350C | 895G | 1447T | 1777C | 1789C | 1976A | 2074C | 2153G | 2527A |
| 272C | 337T | 350C | 895T | 1447C | 1777C | 1789C | 1976A | 2074C | 2153G | 2527G | |
| 272C | 281T | 337T | 350C | 895G | 1447C | 1777C | 1789C | 1976A | 2074C | 2153C | 2527G |
| 272C | 281T | 337T | 350C | 895G | 1447C | 1777C | 1789C | 1976A | 2074T | 2153G | 2527G |
| 272C | 281C | 337T | 350C | 895G | 1777C | 1789C | 1976A | 2074C | 2153G | 2527G | |
| 272C | 281T | 337T | 350C | 895G | 1447C | 1777C | 1789C | 1976A | 2074C | 2153G | 2527G |
| 350T | 895G | 1447T | 1777C | 1789C | 1976A | 2074C | 2153G | 2527G | | | |
| 272T | 895G | 1777C | 1789C | 1976A | 2074T | 2153G | 2527G | | | | |
| 272C | 337T | 350C | 895G | 1777C | 1789T | 1976A | 2074C | 2527G | | | |
| 895G | 1777T | 1789C | 1976K | 2074C | 2153G | 2527G | | | | | |
| 272C | 281T | 337T | 350C | 895G | 1447T | 1777C | 1789C | 1976C | 2074C | 2153G | 2527G |
| 272C | 281T | 337T | 350C | 895G | 1447T | 1777C | 1789C | 1976A | 2074C | 2153G | 2527G |
| 272C | 281T | 337T | 895G | 1447C | 1777C | 1789C | 1976A | 2074C | 2153G | 2527A | |
| 337C | 895G | 1447C | 1777C | 1789C | 1976A | 2074C | 2153G | 2527G | | | |
| 272C | 281T | 337T | 350C | 895G | 1447C | 1777C | 1789C | 1976A | 2074C | 2153C | 2527A |
| 272C | 281C | 337T | 350C | 895T | 1447C | 1777C | 1789C | 1976A | 2074C | 2153G | 2527G |
| 272T | 337C | 350T | 895G | 1447C | 1777C | 1789C | 1976A | 2074C | 2153G | 2527G | |
| 272C | 281C | 337T | 350C | 895G | 1447C | 1777C | 1789C | 1976A | 2074C | 2153G | 2527G |
| 272C | 281T | 337T | 895G | 1447C | 1777C | 1789C | 1976A | 2074C | 2153G | 2527A | |
| 272T | 281C | 337C | 350T | 895T | 1447C | 1777C | 1789C | 1976A | 2074C | 2153G | 2527G |
| 350C | 895G | 1447C | 1777C | 1789C | 1976A | 2074C | 2153G | 2527G | | | |
| 272C | 281T | 337T | 350C | 895G | 1447C | 1777C | 1789C | 1976C | 2074C | 2153C | 2527G |
| 272T | 281C | 337C | 350C | 895G | 1447C | 1777C | 1789C | 1976A | 2074C | 2153G | 2527G |
| 272T | 281C | 337C | 350T | 895G | 1447T | 1777C | 1789C | 1976A | 2074T | 2153G | 2527G |
| 272T | 281C | 337C | 350T | 895G | 1447C | 1777C | 1789C | 1976A | 2074C | 2153G | 2527G |
| 272T | 281C | 337C | 350T | 895G | 1447C | 1777T | 1789C | 1976A | 2074C | 2153G | 2527G |
| 272T | 281C | 337C | 350T | 895G | 1447T | 1777C | 1789C | 1976A | 2074C | 2153G | 2527G |
| 272T | 281C | 337C | 350T | 895G | 1447C | 1777C | 1789C | 1976A | 2074T | 2153G | 2527G |
| 272C | 281T | 337T | 895G | 1447T | 1777C | 1789C | 1976A | 2074C | 2153G | 2527G | |
| 272C | 337T | 350C | 895G | 1447C | 1777C | 1789T | 1976A | 2074C | 2153C | 2527G | |

| | | |
|---|---|---|
| 272 | (−348)C > T | 5′ |
| 281 | (−339)T > C | 5′ |
| 337 | (−283)T > C | 5′ |
| 350 | (−270)C > T | 5′ |
| 895 | 276G > T | Silent |
| 1447 | 828C > T | Silent |
| 1777 | 1158C > T | Silent |
| 1789 | 1170C > T | Silent |
| 1976 | 1357A > C | I453L |
| 2074 | 1455T > C | Silent |
| 2153 | 1534G > C | V512L |
| 2527 | 1908G > A | 3′ |
| 2868 | 2249A > T | 3′ |

| | | | | |
|---|---|---|---|---|
| TPP2 | M55169 | 190470 | GEN-35U | Homo sapiens tripeptidyl |

peptidase II mRNA, 3′ end

| | | |
|---|---|---|
| 2681 | 2681T > G | F894C |
| 3637 | 3637G > K | 3′ |

| | | | | |
|---|---|---|---|---|
| U55206 | U55206 | 601509 | GEN-35Z | Homo sapiens human gamma- |

glutamyl hydrolase (hGH) mRNA, complete cds

| | | | | |
|---|---|---|---|---|
| 75T | 150G | 511C | 703K | 1161G |
| 75T | 150G | 511C | 703G | 1161A |
| 75T | 150G | 511T | 703A | 1161A |
| 75C | 703A | 1161G | | |
| 75C | 511C | 703A | 1161A | |
| 75T | 150G | 511C | 703A | 1161A |
| 75C | 150G | 511C | 703A | 1161A |
| 75C | 150A | 511C | 1161A | |
| 75T | 150G | 511T | 703A | 1161G |
| 75C | 150G | 511T | 703A | 1161A |
| 75C | 150A | 511C | 703A | 1161A |
| 75C | 150G | 511T | 703A | 1161G |
| 75T | 511C | 703A | | |

| | | |
|---|---|---|
| 75 | 16T > C | C6R |
| 150 | 91G > A | A31T |
| 511 | 452C > T | T151I |
| 703 | 644A > G | N21SS |
| 1161 | 1102A > G | 3′ |

| | | | | |
|---|---|---|---|---|
| X56199 | X56199 | 603881 | GEN-36T | Human XIST, coding sequence ‘a’ |

mRNA (locus DXS399E)

| | | |
|---|---|---|
| 1338 | 1338T > G | 3′ |

| | | | | |
|---|---|---|---|---|
| X56549 | X56549 | 134651 | GEN-370 | Human mRNA for muscle fatty- |

acid-binding protein (FABP)

| | |
|---|---|
| 203A | 342C |
| 203A | 342T |
| 203G | 342C |

| | | |
|---|---|---|
| 203 | 158A > G | K53R |
| 342 | 297C > T | Silent |

| | | | | |
|---|---|---|---|---|
| YWHAB | X57346 | 601289 | GEN-37R | H. sapiens mRNA for HS1 protein |

| | | |
|---|---|---|
| 432 | 60C > A | Silent |
| 1135 | 763T > C | 3′ |

| | | | | |
|---|---|---|---|---|
| X57348 | X57348 | 601290 | GEN-37S | H. sapiens mRNA (clone 9112) |

| | | |
|---|---|---|
| 786 | 621C > T | Silent |
| 1317 | 1152C > T | 3′ |
| 1342 | 1177C > T | 3′ |

| | | | | |
|---|---|---|---|---|
| X57522 | X57522 | 170260 | GEN-37W | H. sapiens RING4 cDNA |

| | | | | | |
|---|---|---|---|---|---|
| 1319C | 1465T | 2193G | 2534G | 2598C | 2650C |
| 1465G | 2193G | 2534G | 2598T | 2650C | |
| 1319C | 1465G | 2193G | 2534G | 2598C | 2650C |
| 1319C | 1465G | 2193G | 2534T | 2598C | 2650C |
| 1319T | 1465G | 2534G | 2598C | 2650C | |
| 1319C | 1465G | 2193G | 2534G | 2598C | 2650G |
| 1465G | 2534T | 2598C | 2650C | | |
| 1319T | 1465G | 2193A | 2534G | 2598C | 2650C |
| 1465G | 2193G | 2534G | 2598T | 2650C | |
| 1319C | 1465G | 2534T | 2598T | 2650C | |

| | | |
|---|---|---|
| 1207 | 1177A > G | I393V |
| 1319 | 1289C > T | A430V |
| 1465 | 1435G > T | G479C |
| 2120 | 2090A > G | D697G |
| 2193 | 2163G > A | Silent |
| 2534 | 2504G > T | 3′ |
| 2598 | 2568C > T | 3′ |
| 2650 | 2620G > G | 3′ |

| | | | | |
|---|---|---|---|---|
| X57819 | X57819 | 147220 | GEN-389 | Human rearranged immunoglobulin |

lambda light chain mRNA

| | | |
|---|---|---|
| 499 | 499T > C | C167R |
| 524 | 524G > A | Frame |
| 545 | 545G > A | S182N |
| 558 | 558A > C | Q186H |
| 571 | 571G > A | E19lK |
| 616 | 616C > T | Frame |
| 639 | 639G > A | Silent |
| 695 | 695A > G | Y232C |
| 714 | 714C > T | 3′ |
| 724 | 724C > T | 3′ |

| | | | | |
|---|---|---|---|---|
| M57899 | M57899 | 191740 | GEN-38A | Human bilirubin UDP- |

glucuronosyltransferase isozyme 1 mRNA, complete cds

| | | | | | | |
|---|---|---|---|---|---|---|
| 226G | 1213A | 1443C | 1444G | 1828C | 1956C | 2057C |
| 226G | 1213A | 1443C | 1444G | 1828C | 1956C | 2057G |
| 226A | 1213C | 1443C | 1444G | 1828C | 1956C | 2057C |
| 226G | 1213A | 1443T | 1444G | 1828T | 1956C | 2057G |
| 226G | 1213A | 1443C | 1444G | 1828T | 1956C | 2057G |
| 226G | 1213A | 1443C | 1444A | 1828C | 1956C | 2057C |
| 1213A | 1443C | 1444G | 1828T | 2057C | | |
| 226G | 1213A | 1443C | 1444G | 1828T | 1956G | 2057G |
| 226A | 1213A | 1443C | 1444G | 1828C | 1956C | 2057C |
| 226A | 1213A | 1443C | 1444G | 1828T | 1956G | 2057G |
| 226A | 1213A | 1443C | 1444G | 1828T | 1956C | 2057C |

| | | |
|---|---|---|
| 226 | 211G > A | G71R |
| 1213 | 1198A > C | N400H |
| 1443 | 1428C > T | Silent |
| 1444 | 1429G > A | A477T |
| 1828 | 1813C > T | 3′ |
| 1956 | 1941C > G | 3′ |
| 2057 | 2042C > G | 3′ |

| | | | | |
|---|---|---|---|---|
| GPX3 | X58295 | 138321 | GEN-38S | Human GPx-3 mRNA for plasma |

glutathione peroxidase

| | | | |
|---|---|---|---|
| 821 | 773C > T | | 3′ |
| 979 | 931G > A | 3′ | |
| 1187 | 1139T > G | 3′ | |
| 1354 | 1306C > T | 3′ | |
| 1443 | 1395C > T | 3′ | |
| 1516 | 1468C > A | 3′ | |
| 1581 | 1533C > T | 3′ | |

| | | | | |
|---|---|---|---|---|
| PXMP1 | X58528 | 170995 | GEN-392 | Human PMP7O mRNA for a |

peroxisomal membrane protein

| | | |
|---|---|---|
| 2375 | 2351C > T | 3′ |

| | | | | |
|---|---|---|---|---|
| M58664 | M58664 | 103000 | GEN-395 | Homo sapiens CD24 signal |

transducer mRNA, complete cds

| | | |
|---|---|---|
| 226 | 170C > T | A57V |
| 570 | 514A > T | 3′ |
| 1109 | 1053A > G | 3′ |
| 1334 | 1278C > G | 3′ |
| 1345 | 1289T > C | 3′ |
| 1374 | 1318C > T | 3′ |
| 1403 | 1347C > T | 3′ |
| 1408 | 1352T > G | 3′ |
| 1415 | 1359C > A | 3′ |
| 1677 | 1621A > G | 3′ |

| | | | | |
|---|---|---|---|---|
| BTK | X58957 | 300300 | GEN-39A | H. sapiens atk mRNA for |

agammaglobulinaemia tyrosine kinase

| | | |
|---|---|---|
| 2228 | 2096A > C | 3′ |
| 2304 | 2172A > G | 3′ |

| | | | | |
|---|---|---|---|---|
| U59185 | U59185 | 603878 | GEN-39I | Human putative monocarboxylate |

transporter (MCT) mRNA, complete cds

| | |
|---|---|
| 863G | 972A |
| 863A | 972C |
| 863A | 972A |

| | | |
|---|---|---|
| 863 | 681A > G | Silent |
| 972 | 790A > C | N264H |
| 2351 | 2169A > G | 3′ |

| | | | | |
|---|---|---|---|---|
| X60069 | X60069 | 231950 | GEN-3AJ | Human mRNA for pancreatic gamma- |

glutamyltransferase

| | | | | | | |
|---|---|---|---|---|---|---|
| 1060G | 1173C | 1310G | 1399T | 1598G | 1641G | 2148G |
| 1060G | 1173T | 1310A | 1399C | 1598G | 1641G | 2148G |
| 1060G | 1173C | 1310G | 1399C | 1598G | 1641G | 2148A |
| 1060G | 1173T | 1310G | 1399C | 1598G | 1641G | 2148G |
| 1060G | 1173C | 1310G | 1399C | 1598G | 1641G | 2148G |
| 1060G | 1173C | 1310G | 1399C | 1598A | 1641G | 2148G |
| 1060A | 1173C | 1310G | 1399C | 1598G | 1641G | 2148G |
| 1060G | 1173C | 1310A | 1399C | 1598G | 1641G | 2148G |
| 1060G | 1173T | 1399C | 1641G | 2148A | | |
| 1060G | 1173C | 1310A | 1399C | 1598G | 1641G | 2148A |
| 1060G | 1173T | 1399C | 1598G | 1641A | 2148G | |
| 1060G | 1173T | 1310A | 1399C | 1641G | 2148A | |
| 1060G | 1173T | 1310A | 1399C | 1598G | 1641G | |
| 1173C | 1310G | 1399C | 1598G | 1641G | 2148G | |
| 1060G | 1173T | 1310A | 1399C | 1598G | 1641A | 2148G |
| 1060G | 1173T | 1310A | 1399C | 1598G | 1641A | |

| | | |
|---|---|---|
| 102 | (−257)G > A | 5′ |
| 336 | (−23)C > T | 5′ |
| 1060 | 702G > A | Silent |
| 1173 | 815C > T | A272V |
| 1310 | 952G > A | E318K |
| 1399 | 1041C > T | Silent |
| 1409 | 1051G > T | A3S1S |
| 1482 | 1124C > T | T375M |
| 1591 | 1233G > A | Silent |
| 1598 | 1240G > A | G414R |
| 1624 | 1266C > T | Silent |
| 1637 | 1279C > A | P427T |
| 1641 | 1283G > A | S428N |
| 1651 | 1293C > T | Silent |
| 1662 | 1304T > C | V435A |
| 1783 | 1425A > G | Silent |
| 1794 | 1436C > T | T479M |
| 1795 | 1437G > A | Silent |
| 1981 | 1623C > T | Silent |
| 2007 | 1649C > T | T550M |
| 2031 | 1673C > T | 5558L |
| 2047 | 1689C > T | Silent |
| 2147 | 1789C > T | 3′ |
| 2148 | 1790G > A | 3′ |
| 2176 | 1818C > T | 3′ |
| 2224 | 1866C > A | 3′ |

| | | | | |
|---|---|---|---|---|
| TCN2 | M60396 | 275350 | GEN-3AX | Human transcobalamin II (TCII) |

mRNA,complete cds

| | | |
|---|---|---|
| 1164 | 1127C > T | S376L |
| 1765 | 1728T > C | 3′ |

| | | | | |
|---|---|---|---|---|
| U60519 | U60519 | 601762 | GEN-3AZ | Human apoptotic cysteine |

protease Mch4 (Mch4) mRNA, complete cds

| | | | | |
|---|---|---|---|---|
| 1246A | 1355A | 2606A | 2651A | |
| 1246G | 1355A | 2605A | 2606A | 2651A |
| 1246G | 1355A | 2605G | 2606G | 2651A |
| 2246G | 1355G | 2605G | | |
| 1246G | 1355A | 2605G | 2606A | 2651A |
| 1246G | 1355A | 2605G | 2606A | 2651G |
| 1246G | 1355A | 2605G | 2606G | 2651G |
| 1246G | 1355G | 2605G | 2606A | 2651A |
| 1246A | 1355A | 2605G | 2606A | 2651A |

| | | |
|---|---|---|
| 304 | 157G > A | E53K |
| 324 | 177A > G | Silent |
| 1246 | 1099G > A | V367I |
| 1355 | 1208A > G | Y403C |
| 2605 | 2458A > G | 3′ |
| 2606 | 2459A > G | 3′ |
| 2651 | 2504A > G | 3′ |

| | | | | |
|---|---|---|---|---|
| X60592 | X60592 | 109535 | GEN-3B0 | Human CDw40 mRNA for nerve |

growth factor receptor-related B-lymphocyte activation molecule

| | |
|---|---|
| 418C | 726G |
| 418T | 726C |
| 418C | 726C |

| | | |
|---|---|---|
| 418 | 371C > T | S124L |
| 726 | 679C > G | P227A |

| | | | | |
|---|---|---|---|---|
| NFKB2 | X61498 | 164012 | GEN-3BW | H. sapiens mRNA for NF-kB subunit |

| | | | | |
|---|---|---|---|---|
| 1375G | 1814G | 2203G | 2218G | 2254C |
| 1375G | 1814G | 2203G | 2218C | 2254T |
| 1375G | 1814G | 2203G | 2218C | 2254C |
| 1375T | 1814G | 2203G | 2218C | 2254C |
| 1375G | 1814G | 2203A | 2218C | 2254C |
| 1375G | 1814C | 2203G | 2218C | 2254C |
| 1375T | 1814C | 2203G | 2218C | 2254C |
| 1375T | 1814G | 2203G | 2218G | 2254C |
| 1375G | 1814G | 2203A | 2218G | 2254C |

| | | |
|---|---|---|
| 1375 | 1212GT | Silent |
| 1814 | 1651G > C | D551H |
| 2203 | 2040C > A | Silent |
| 2218 | 2055C > G | Silent |
| 2254 | 2091C > T | Silent |
| 2457 | 2294C > T | P765L |

| | | | | |
|---|---|---|---|---|
| M61855 | M61855 | 601130 | GEN-3C1 | Human cytochrome P4502C9 |

(CYP2C9) mRNA, clone 25

| | |
|---|---|
| 442T | 1087A |
| 442C | 1087A |
| 442C | 1087C |

| | | |
|---|---|---|
| 442 | 442C > T | 3′ |
| 852 | 852T > A | 3′ |
| 1085 | 1085A > G | 3′ |
| 1087 | 1087C > A | 3′ |
| 1437 | 1437T > A | 3′ |

| | | | | |
|---|---|---|---|---|
| X62572 | X62572 | 146790 | GEN-3CL | H. sapiens RNA for Fc receptor, |
| PC23 | | | | |

| | | |
|---|---|---|
| 967 | 967T > C | 3′ |
| 1240 | 1240A > G | 3′ |
| 1300 | 1300C > T | 3′ |
| 1542 | 1542G > C | 3′ |
| 1560 | 1560C > A | 3′ |
| 1709 | 1709T > G | 3′ |
| 1931 | 1931A > T | 3′ |
| 2032 | 2032G > A | 3′ |
| 2136 | 2136G > A | 3′ |
| 2176 | 2176C > T | 3′ |
| 2201 | 2201G > A | 3′ |

| | | | | |
|---|---|---|---|---|
| X62744 | X62744 | 142855 | GEN-3CQ | Human RING6 mRNA for HLA class |

II alpha chain-like product

| | | |
|---|---|---|
| 541 | 496G > A | V166I |
| 674 | 629G > A | R210H |
| 750 | 705G > C | Silent |
| 1081 | 1036A > T | 3′ |

| | | | | |
|---|---|---|---|---|
| X63359 | X63359 | 600070 | GEN-3DC | H. sapiens UGT2BIO mRNA for udp |

glucuronosyltransferase

| | |
|---|---|
| 2219T | 2422A |
| 2219T | 2422G |
| 2219C | 2422G |

| | | |
|---|---|---|
| 1516 | 1506C > T | Silent |
| 2219 | 2209T > C | 3′ |
| 2422 | 2412G > A | 3′ |
| 2714 | 2704G > A | 3′ |

| | | | | |
|---|---|---|---|---|
| TCRB | X63456 | 186930 | GEN-3DG | H. sapiens mRNA for T-cell |

antigen receptor beta-chain

| | | |
|---|---|---|
| 421 | 411G > C | K137N |
| 496 | 486G > A | Silent |
| 516 | 506T > A | F169Y |
| 520 | 510C > T | Silent |
| 580 | 570A > G | Silent |
| 754 | 744C > T | Silent |
| 805 | 795T > C | Silent |
| 811 | 801G > C | Silent |
| 813 | 803T > A | V268E |
| 817 | 807C > T | Silent |
| 860 | 850C > T | Silent |
| 878 | 868C > A | L290M |

| | | | | |
|---|---|---|---|---|
| X64177 | X64177 | 156351 | GEN-3EQ | H. sapiens mRNA for |

metallothionein

| | | |
|---|---|---|
| 63 | 40G > A | A14T |
| 90 | 67A > G | K23E |
| 125 | 102C > T | Silent |
| 131 | 108T > C | Silent |
| 168 | 145A > G | I49V |
| 182 | 159G > A | Silent |

| | | | | |
|---|---|---|---|---|
| CPA1 | X67318 | 114850 | GEN-3HJ | H. sapiens mRNA for |

procarboxypeptidase A1

| | | |
|---|---|---|
| 172 | 165G > C | Silent |
| 498 | 491C > G | T164R |
| 629 | 622G > A | A208T |

| | | | | |
|---|---|---|---|---|
| X67699 | X67699 | 114280 | GEN-3HP | H. sapiens HES mRNA for CDw52 |
| antigen | | | | |

| | | |
|---|---|---|
| 143 | 119G > A | S40N |
| 147 | 123G > A | M41I |

| | | | | |
|---|---|---|---|---|
| X68836 | X68836 | 601468 | GEN-31R | H. sapiens mRNA for S- |

adenosylmethionine synthetase

| | |
|---|---|
| 857G | 878T |
| 857G | 878C |
| 857C | 878T |

| | | |
|---|---|---|
| 240 | 175G > A | V59I |
| 857 | 792G > C | Silent |
| 878 | 813T > C | Silent |

| | | | | |
|---|---|---|---|---|
| M69043 | M69043 | 164008 | GEN-3IZ | Homo sapiens MAD-3 mRNA encoding |

IkB-like activity, complete cds

| | |
|---|---|
| 1050T | 1174A |
| 1050C | 1174G |

| | | |
|---|---|---|
| 400 | 306T > C | Silent |
| 1050 | 956T > C | 3′ |
| 1119 | 1025G > A | 3′ |
| 1174 | 1080A > G | 3′ |

| | | | | |
|---|---|---|---|---|
| ARNT | M69238 | 126110 | GEN-3JH | Human aryl hydrocarbon receptor |

nuclear translocator (ARNT) mRNA, complete cds

| | | |
|---|---|---|
| 623 | 567G > C | Silent |

| | | | | |
|---|---|---|---|---|
| X71440 | X71440 | 264470 | GEN-3KS | H. sapiens mRNA for peroxisomal |

acyl-CoA oxidase

| | |
|---|---|
| 949G | 1333T |
| 949C | 1333C |
| 949C | 1333T |

| | | |
|---|---|---|
| 949 | 936G > C | M312I |
| 1333 | 1320T > C | Silent |

| | | | | |
|---|---|---|---|---|
| GPX4 | X71973 | 138322 | GEN-3L1 | H. sapiens GPx-4 mRNA for |

phospholipid hydroperoxide glutathione peroxidase

| | | |
|---|---|---|
| 718 | 638T > C | 3′ |
| 837 | 757C > A | 3′ |
| 882 | 802A > C | 3′ |

| | | | | |
|---|---|---|---|---|
| RGS1 | X73427 | 600323 | GEN-3M6 | H. sapiens 1r20 mRNA for alpha |

helical basic phosphoprotein

| | | |
|---|---|---|
| 247 | 233C > T | A78V |

| | | | | |
|---|---|---|---|---|
| MHC2TA | X74301 | 600005 | GEN-3N5 | H. sapiens mRNA for MHC class II |

transactivator

| | | |
|---|---|---|
| 1614 | 1499C > G | A500G |
| 3759 | 3644G > A | 3′ |
| 4422 | 4307T > C | 3′ |

| | | | | |
|---|---|---|---|---|
| ALDH3 | M74542 | 100660 | GEN-3N9 | Human aldehyde dehydrogenase |

type III (ALDHIII) mRNA, complete cds

| | | |
|---|---|---|
| 1616 | 1574A > G | 3′ |

| | | | | |
|---|---|---|---|---|
| U34252 | U34252 | 602733 | GEN-3O5 | Human gamma-aminobutyraldehyde |

dehydrogenase mRNA, complete cds

| | |
|---|---|
| 1683G | 2471A |
| 1683A | 2471A |
| 1683G | 2471C |

| | | |
|---|---|---|
| 1683 | 1306G > A | E436K |
| 2417 | 2040G > A | 3′ |
| 2471 | 2094A > C | 3′ |
| 2674 | 2297A > C | 3′ |
| 2676 | 2299A > C | 3′ |

| | | | | |
|---|---|---|---|---|
| MTP | X75500 | 157147 | GEN-3O7 | H. sapiens mRNA for microsomal |

triglyceride transfer protein

| | | | | | | | | | | | |
|---|---|---|---|---|---|---|---|---|---|---|---|
| 63C | 148G | 309G | 407T | 477C | 521A | 546T | 754C | 915G | 957C | 1175A | 2049C |
| 63C | 148A | 309G | 477T | 521A | 546T | 754C | 915C | 957C | 1175A | 2049C | |
| 63C | 148G | 309G | 407T | 477C | 521G | 546C | 754C | 915G | 957C | 1175A | 2049C |
| 63C | 148G | 309G | 407C | 477C | 521A | 546T | 915G | 957C | 1175A | 2049C | |
| 63C | 148G | 309G | 407T | 477C | 754C | 915G | 957A | 1175A | 2049C | | |
| 63C | 148G | 309G | 407C | 477C | 546C | 754C | 915G | 957C | 1175A | 2049C | |
| 63C | 148G | 309G | 407T | 546C | 754C | 915C | 957C | 1175A | 2049C | | |
| 63C | 148G | 309G | 477T | 521A | 546T | 754C | 915G | 957C | 1175A 2049C | | |
| 63C | 148G | 407C | 521A | 546T | 754C | 915C | 957C | 1175A | 2049C | | |
| 148G | 309G | 407T | 477C | 521A | 546T | 754C | 915C | 957C | 1175A | 2049C | |
| 63G | 148G | 309G | 407T | 477C | 521A | 754C | 915G | 957C | 2049T | | |
| 63G | 148G | 309G | 407T | 477C | 521A | 546T | 754C | 915G | 957C | 1175A | 2049C |
| 63C | 148G | 309G | 407T | 477C | 521A | 546T | 754C | 915G | 957C | 1175A | 2049T |
| 148G | 309G | 407T | 521A | 546T | 754C | 915C | 957C | 1175A | 2049C | | |
| 148G | 309G | 407T | 477C | 521A | 546T | 754C | 915G | 957C | 1175C | 2049C | |
| 63C | 148G | 309G | 407C | 477C | 521A | 546T | 754C | 915G | 957C | 1175A | 2049T |
| 63C | 148G | 309G | 407T | 477T | 521A | 546T | 754C | 915C | 957C | 1175A | 2049C |
| 63C | 148G | 309G | 407T | 477C | 521A | 546C | 754C | 915G | 957C | 1175A | 2049C |
| 63G | 148G | 309G | 407C | 477C | 521A | 546T | 754C | 915G | 957A | 1175C | 2049C |
| 63C | 148G | 309G | 407C | 477C | 521G | 546C | 754C | 915G | 957C | 1175A | 2049C |
| 63G | 148G | 309G | 407T | 477C | 521A | 546C | 754C | 915G | 957C | 1175C | 2049T |
| 63C | 148G | 309G | 407T | 477C | 521G | 546C | 754C | 915G | 957C | 2049C | |
| 63C | 148G | 309G | 407T | 477C | 521A | 546T | 754C | 915G | 957A | 1175C | 2049C |
| 63C | 148A | 309G | 407C | 477T | 521A | 546T | 754C | 915C | 957C | 1175A | 2049C |
| 63C | 148G | 309G | 407T | 477C | 521A | 546T | 754C | 915G | 957C | 1175A | |
| 63C | 148G | 309G | 407C | 477C | 521A | 546T | 754C | 915G | 957C | 1175A | 2049C |
| 63C | 148G | 309G | 407C | 477C | 521A | 546T | 754C | 915C | 957C | 1175A | 2049C |
| 63G | 148G | 309G | 407T | 477C | 521A | 546T | 754C | 915G | 957C | 1175C | 2049C |
| 63G | 148G | 309G | 407T | 477C | 521A | 546T | 754C | 915C | 957C | 1175A | 2049C |
| 63C | 148G | 309G | 407T | 477C | 521G | 546C | 754C | 915G | 957A | 1175A | 2049C |
| 63C | 148G | 309G | 407C | 477T | 521A | 546T | 754C | 915G | 957C | 1175A | 2049C |
| 63C | 148G | 309G | 407T | 477C | 521G | 546C | 754C | 915C | 957C | 1175A | 2049C |
| 63G | 148G | 309G | 407T | 477C | 521A | 546T | 754C | 915C | 957C | 1175C | 2049C |
| 63C | 148G | 309G | 407C | 477C | 521A | 546C | 754C | 915G | 957C | 1175A | 2049C |

| | | |
|---|---|---|
| 63 | 39C > G | Silent |
| 148 | 124G > A | V42I |
| 309 | 285G > C | Q95H |
| 407 | 383T > C | I128T |
| 477 | 453T > C | Silent |
| 521 | 497A > G | N166S |
| 546 | 522T > C | Silent |
| 754 | 730C > G | Q244E |
| 915 | 891C > G | H297Q |
| 957 | 933C > A | Silent |
| 1175 | 1151A > C | D384A |
| 1847 | 1823T > G | F608C |
| 2049 | 2025C > T | Silent |
| 3231 | 3207G > A | 3′ |

| | | | | |
|---|---|---|---|---|
| X75535 | X75535 | 600279 | GEN-3O8 | H. sapiens mRNA for PxF protein |

| | | |
|---|---|---|
| 1808 | 1798A > G | 3′ |
| 3066 | 3056G > C | 3′ |
| 3263 | 3253G > T | 3′ |

| | | | | |
|---|---|---|---|---|
| U76368 | U76368 | 601872 | GEN-3OU | Human cationic amino acid |

transporter-2A (ATRC2) mRNA, complete cds

| | | |
|---|---|---|
| 1338C | 1445A | 1904G |
| 1338T | 1904C | |
| 1338C | 1445C | 1904G |
| 1338A | 1445A | 1904C |
| 1338A | 1445C | 1904G |
| 1338T | 1904G | |
| 1338A | 1445A | 1904G |
| 1338T | 1904C | |
| 1338T | 1904G | |

| | | |
|---|---|---|
| 1338 | 1144A > C | K382Q |
| 1338 | 1144A > T | Frame |
| 1445 | 1251A > C | Silent |
| 1904 | 1710G > C | Silent |

| | | | | |
|---|---|---|---|---|
| LIPA | X76488 | 278000 | GEN-3P2 | H. sapiens mRNA for lysosomal |

acid lipase

| | | | | |
|---|---|---|---|---|
| 191A | 212G | 2186C | 2254T | 2439C |
| 191A | 212A | 2186C | 2254A | 2439C |
| 191C | 212G | 2186C | 2254T | 2439C |
| 191A | 212G | 2186C | 2254T | 2439T |
| 191A | 212G | 2186G | 2254T | 2439C |
| 191C | 212G | 2186C | 2254A | 2439C |
| 191A | 212A | 2186C | 2254T | 2439C |
| 191A | 212A | 2186C | 2439T | |
| 191A | 212G | 2186C | 2254A | 2439C |
| 191A | 212A | 2186C | 2254A | 2439T |

| | | |
|---|---|---|
| 191 | 46A > C | T16P |
| 212 | 67G > A | G23R |
| 967 | 822G > A | M274I |
| 1531 | 1386C > T | 3′ |
| 2186 | 2041C > G | 3′ |
| 2254 | 2109A > T | 3′ |
| 2439 | 2294C > T | 3′ |

| | | | | |
|---|---|---|---|---|
| U76560 | U76560 | 601757 | GEN-3P4 | Human peroxisome targeting |

signal 2 receptor (Pex7) mRNA, complete cds

| | | |
|---|---|---|
| 1326 | 1278T > G | 3′ |

| | | | | |
|---|---|---|---|---|
| M77829 | M77829 | 107776 | GEN-3QJ | Human channel-like integral |

membrane protein (CHIP28) mRNA, complete cds

| | | |
|---|---|---|
| 172 | 134C > T | A45V |
| 1249 | 1211C > G | 3′ |

| | | | | |
|---|---|---|---|---|
| ID1 | X77956 | 600349 | GEN-3QL | H. sapiens Id1 mRNA |

| | | |
|---|---|---|
| 380 | 345G > A | Silent |
| 382 | 347C > A | T116N |
| 842 | 807A > C | 3′ |
| 851 | 816G > A | 3′ |

| | | | | |
|---|---|---|---|---|
| YWHAH | X78138 | 113508 | GEN-3QU | H. sapiens 14-3-3 eta subtype |
| mRNA | | | | |

| | | |
|---|---|---|
| 953 | 753A > G | 3′ |
| 960 | 760G > A | 3′ |
| 1387 | 1187C > T | 3′ |

| | | | | |
|---|---|---|---|---|
| S78203 | S78203 | 602339 | GEN-3QY | PEPT 2=H+/peptide cotransporter |

[human, kidney, mRNA Partial, 2685 nt]

| | | | | | | | |
|---|---|---|---|---|---|---|---|
| 171C | 1078C | 1191A | 1255C | 1365C | 1556G | 2226C | 2311G |
| 171C | 1078T | 1191G | 1255T | 1365C | 1556A | 2226C | 2311G |
| 171C | 1078C | 1191G | 1365C | 2226C | 2311G | | |
| 171C | 1078T | 1191G | 1255T | 1365T | 1556A | 2226C | 2311G |
| 171C | 1365C | 2226T | 2311G | | | | |
| 171C | 1078T | 1191G | 1255T | 1365C | 1556G | 2226C | 2311G |
| 171T | 1365C | 2226C | 2311G | | | | |
| 1365C | 2226C | 2311A | | | | | |
| 171C | 1078C | 1191A | 1255T | 1365C | 1556G | 2226C | 2311G |
| 171C | 1078C | 1191A | 1255C | 1365C | 1556G | 2226T | 2311G |
| 1078C | 1191A | 1255C | 1365C | 1556G | 2226C | 2311A | |
| 171C | 1078C | 1191A | 1255T | 1365C | 2226C | 2311G | |
| 171T | 1078C | 1191A | 1255C | 1365C | 1556G | 2226C | 2311G |
| 171C | 1078C | 1191G | 1255T | 1365C | 1556A | 2226C | 2311G |
| 1078T | 1191G | 1255T | 1365C | 1556A | 2226C | 2311G | |

| | | |
|---|---|---|
| 171 | 141C > T | Silent |
| 1078 | 1048C > T | L350F |
| 1191 | 1161A > G | Silent |
| 1255 | 1225C > T | P409S |
| 1365 | 1335C > T | Silent |
| 1556 | 1526G > A | R509K |
| 2226 | 2196C > T | 3′ |
| 2311 | 2281G > A | 3′ |

| | | | | |
|---|---|---|---|---|
| X79389 | X79389 | 600436 | GEN-3T7 | H. sapiens GSTT1 mRNA |

| | | |
|---|---|---|
| 824 | 824T > C | 3′ |

| M80244 | | M80244 | | 600182 | | GEN-3UJ | | Human E16 mRNA, complete cds | | | | |
|---|---|---|---|---|---|---|---|---|---|---|---|---|
| | 1324A | 1473C | 1493G | 1614G | 1862G | 1918A | 2102T | 2728G | 2811C | 2917G | 2933C | 2992G |
| | 3538T | 3872G | | | | | | | | | | |
| | 1111C | 1119C | 1324T | 1473C | 1614G | 1862G | 1918C | 2102T | 2591A | 2728G | 2811C | 2917G |
| | 2933C | 2992A | | | | | | | | | | |
| | 1111C | 1119C | 1324T | 1493G | 1862G | 1918C | 2102T | 2728G | 2811C | 2917G | 2933A | 2992G |
| | 3538T | 3872G | | | | | | | | | | |
| | 1324T | 1493G | 1614G | 1862G | 2728T | 2811C | 2917G | 2933C | 3538T | 3872G | | |
| | 1324A | 1473G | 1493G | 1614G | 1862G | 2102T | 2591A | 2728G | 2811C | 2917G | 2933C | 2992G |
| | 3538T | 3872G | | | | | | | | | | |
| | 1111C | 1119C | 1324T | 1473C | 1493G | 1614G | 2591G | 2728G | 2811C | 2917G | 2933C | 2992G |
| | 3538C | 3872G | | | | | | | | | | |
| | 1111C | 1119C | 1324T | 1493G | 1614G | 1862T | 1918C | 2102T | 2728G | 2811C | 2917G | 2933C |
| | 2992G | 3538T | | | | | | | | | | |
| | 1111C | 1119C | 1324T | 1473C | 1493G | 1614A | 1862G | 1918C | 2102T | 2591G | 2728G | 2811C |
| | 2917G | 2933C | 2992G | 3538T | | | | | | | | |
| | 1324A | 1473C | 1493G | 1614G | 1862G | 1918C | 2102T | 2728G | 2811C | 2917G | 2933C | 2992G |
| | 3538T | 3872G | | | | | | | | | | |
| | 1111C | 1119C | 1324T | 1473C | 1493G | 1614G | 1862G | 2591G | 2811C | 2917G | 2933C | 3538T |
| | 3872A | | | | | | | | | | | |
| | 1111C | 1119C | 1324T | 1473C | 1493G | 1614G | 1862G | 1918C | 2102T | 2591G | 2728G | 2811C |
| | 2917G | 2933C | 2992G | 3538T | 3872G | | | | | | | |
| | 1111C | 1119C | 1324T | 1473C | 1493G | 1614G | 1862G | 1918C | 2102T | 2591G | 2728G | 2811C |
| | 2917A | 2933C | 2992G | | | | | | | | | |
| | 1111C | 1119C | 1324T | 1473C | 1614G | 1862G | 1918C | 2102T | 2591A | 2728G | 2811C | 2917G |
| | 2933C | 2992G | 3538T | | | | | | | | | |
| | 1111T | 1119C | 1324T | 1493G | 1614G | 1862G | 1918C | 2102T | 2728G | 2811C | 2917G | 2933C |
| | 2992G | 3538T | 3872G | | | | | | | | | |
| | 1111C | 1119C | 1324T | 1473G | 1493G | 1614G | 1862G | 1918C | 2102T | 2591A | 2728G | 2811T |
| | 2917G | 2933C | 2992G | | | | | | | | | |
| | 1111C | 1119T | 1324T | 1473C | 1493G | 1614G | 1862G | 1918C | 2102T | 2591G | 2728G | 2811C |
| | 2917G | 2933C | 2992G | 3538T | 3872G | | | | | | | |
| | 1111C | 1119C | 1324T | 1473G | 1493G | 1614G | 1862G | 1918C | 2102T | 2591A | 2728G | 2811C |
| | 2917G | 2933C | 2992G | 3538T | | | | | | | | |
| | 1111C | 1119C | 1324T | 1473C | 1493G | 1614G | 1862T | 1918C | 2102T | 2591G | 2728G | 2811C |
| | 2917G | 2933C | 2992G | 3538T | | | | | | | | |
| | 1111C | 1119C | 1324T | 1473C | 1493G | 1614G | 1862G | 1918C | 2102T | 2591G | 2728G | 2811C |
| | 2917G | 2933C | 2992G | | | | | | | | | |
| | 1111C | 1119C | 1324T | 1473C | 1493G | 1614A | 1862G | 1918C | 2102T | 2591G | 2728G | 2811C |
| | 2917G | 2933A | 2992G | 3538T | 3872G | | | | | | | |
| | 1324A | 1473C | 1493G | 1614G | 1862G | 1918A | 2102T | 2591A | 2728G | 2811C | 2917G | 2933C |
| | 2992G | 3538T | 3872G | | | | | | | | | |
| | 1111C | 1119C | 1324T | 1473C | 1493G | 1614G | 2591G | 2728G | 2811C | 2917G | 2933C | 2992G |
| | 3538C | 3872G | | | | | | | | | | |
| | 1111C | 1119C | 1324T | 1473C | 1493A | 1614G | 1862G | 1918C | 2102T | 2591A | 2728G | 2811C |
| | 2917G | 2933C | 2992G | 3538T | 3872A | | | | | | | |
| | 1111C | 1119C | 1324T | 1473C | 1493G | 1614A | 1862G | 1918C | 2102T | 2591G | 2728G | 2811C |
| | 2917G | 2933C | 2992G | 3538T | | | | | | | | |
| | 1111C | 1119C | 1324T | 1473C | 1493G | 1614G | 1862G | 1918C | 2102A | 2591G | 2728G | 2811C |
| | 2917G | 2933C | 2992G | | | | | | | | | |
| | 1324A | 1473C | 1493G | 1614G | 1862G | 1918C | 2102T | 2591G | 2728G | 2811C | 2917G | 2933C |
| | 2992G | 3538T | 3872G | | | | | | | | | |
| | 1111C | 1119C | 1324T | 1473C | 1493G | 1614G | 1862G | 1918C | 2102T | 2591A | 2728G | 2811C |
| | 2917G | 2933C | 2992A | | | | | | | | | |
| | 1111C | 1119C | 1324T | 1473C | 1493G | 1614G | 1862G | 1918C | 2102T | 2591G | 2728G | 2811C |
| | 2917A | 2933C | 2992G | | | | | | | | | |
| | 1111C | 1119C | 1324T | 1473G | 1493G | 1614G | 1862G | 1918C | 2102T | 2591A | 2728G | 2811C |
| | 2917G | 2933C | 2992G | | | | | | | | | |
| | 1111C | 1119C | 1324T | 1473C | 1493G | 1614G | 1862G | 1918A | 2102A | 2591G | 2728T | 2811C |
| | 2917G | 2933C | 2992A | 3538T | 3872A | | | | | | | |
| | 1324T | 1473G | 1493G | 1614G | 1862G | 1918C | 2102T | 2591A | 2728T | 2811C | 2917G | 2933C |
| | 2992G | 3538T | 3872G | | | | | | | | | |
| | 1111C | 1119C | 1324T | 1473G | 1493G | 1614G | 1862G | 1918C | 2102T | 2591A | 2728G | 2811T |
| | 2917G | 2933C | 2992G | | | | | | | | | |
| | 1111C | 1119C | 1324T | 1473C | 1493G | 1614G | 1862G | 1918C | 2102T | 2591G | 2728G | 2811C |
| | 2917G | 2933C | 2992G | 3538T | | | | | | | | |
| | 1324A | 1473G | 1493G | 1614G | 1862G | 1918C | 2102T | 2591A | 2728G | 2811C | 2917G | 2933C |
| | 2992G | 3538T | 3872G | | | | | | | | | |
| | 1111T | 1119C | 1324T | 1473C | 1493G | 1614G | 1862G | 1918C | 2102T | 2591G | 2728G | 2811C |
| | 2917G | 2933C | 2992G | 3538T | 3872G | | | | | | | |

| | | |
|---|---|---|
| 202 | (−109)G > C | 5′ |
| 520 | 210T > C | Silent |
| 1111 | 801C > T | 3′ |
| 1119 | 809C > T | 3′ |
| 1185 | 875G > A | 3′ |
| 1324 | 1014T > A | 3′ |
| 1473 | 1163C > G | 3′ |
| 1493 | 1183A > G | 3′ |
| 1614 | 1304G > A | 3′ |
| 1692 | 1382C > T | 3′ |
| 1862 | 1552G > T | 3′ |
| 1918 | 1608C > A | 3′ |
| 2102 | 1792T > A | 3′ |
| 2591 | 2281A > G | 3′ |
| 2728 | 2418G > T | 3′ |
| 2811 | 2501C > T | 3′ |
| 2917 | 2607G > A | 3′ |
| 2933 | 2623C > A | 3′ |
| 2992 | 2682A > G | 3′ |
| 3138 | 2828G > C | 3′ |
| 3538 | 3228T > C | 3′ |
| 3872 | 3562G > A | 3′ |

| | | | | |
|---|---|---|---|---|
| M80462 | M80462 | 112205 | GEN-3US | Human MB-1 mRNA, complete cds |

| | | |
|---|---|---|
| 241 | 205G > A | V69I |

| | | | | |
|---|---|---|---|---|
| CD79B | M80461 | 147245 | GEN-3UT | Human B29 mRNA, complete cds |

| | | |
|---|---|---|
| 795 | 781C > T | 3′ |
| 804 | 790C > A | 3′ |
| 1033 | 1019C > T | 3′ |

| | | | | |
|---|---|---|---|---|
| U81375 | U81375 | 602193 | GEN-3VO | Human placental equilibrative |

nucleoside transporter 1 (hENT1) mRNA, complete cds

| | | | |
|---|---|---|---|
| 1466G | 1989G | 1996C | 2045T |
| 1466G | 1989A | 1996C | |
| 1466A | 1989G | 1996C | 2045C |
| 1466G | 1989G | 1996T | 2045C |
| 1466G | 1989G | 1996C | 2045C |
| 1466A | 1996C | | |
| 1466G | 1996C | | |
| 1466G | 1989A | 1996C | 2045C |

| | | |
|---|---|---|
| 1466 | 1288G > A | A430T |
| 1989 | 1811G > A | 3′ |
| 1996 | 1818C > T | 3′ |
| 2045 | 1867T > C | 3′ |

| | | | | |
|---|---|---|---|---|
| M81590 | M81590 | 182131 | GEN-3VZ | Serotonin receptor 5HT-1B, cDNA |

| | | |
|---|---|---|
| 190C | 432T | 922C |
| 190C | 432T | 922G |
| 190T | 432T | 922C |
| 190C | 432G | 922G |

| | | |
|---|---|---|
| 190 | 129C > T | Silent |
| 432 | 371T > G | F124C |
| 922 | 861G > C | Silent |
| 1241 | 1180G > A | 3′ |

| | | | | |
|---|---|---|---|---|
| IGHG3 | X81695 | 147120 | GEN-3W4 | H. sapiens rearranged IgG VH-D- |

JH-Hinge-CH2-CH3 region

| | | |
|---|---|---|
| 528 | 528C > T | 3′ |
| 534 | 534C > T | 3′ |
| 594 | 594T > C | 3′ |
| 601 | 601A > C | 3′ |
| 668 | 668A > T | 3′ |
| 726 | 726T > C | 3′ |
| 796 | 796G > A | 3′ |
| 804 | 804G > A | 3′ |
| 827 | 827A > T | 3′ |
| 828 | 828C > T | 3′ |
| 842 | 842C > T | 3′ |
| 849 | 849G > T | 3′ |
| 853 | 853A > C | 3′ |
| 900 | 900T > C | 3′ |
| 905 | 905G > A | 3′ |
| 916 | 916G > A | 3′ |
| 957 | 957G > C | 3′ |
| 963 | 963G > A | 3′ |
| 970 | 970G > A | 3′ |
| 973 | 973C > G | 3′ |
| 999 | 999C > T | 3′ |
| 1002 | 1002T > C | 3′ |
| 1012 | 1012A > C | 3′ |
| 1045 | 1045G > A | 3′ |
| 1050 | 1050C > T | 3′ |
| 1073 | 1073C > G | 3′ |
| 1075 | 1075C > T | 3′ |
| 1088 | 1088A > T | 3′ |
| 1092 | 1092G > A | 3′ |
| 1113 | 1113C > T | 3′ |
| 1130 | 1130G > C | 3′ |
| 1137 | 1137G > A | 3′ |

| | | | | |
|---|---|---|---|---|
| M81757 | M81757 | 603474 | GEN-3W6 | H. sapiens S19 ribosomal protein |

mRNA, complete cds

| | | |
|---|---|---|
| 276 | 254A > T | N85I |
| 338 | 316G > A | A106T |
| 496 | 474G > A | 3′ |

| | | | | |
|---|---|---|---|---|
| U81800 | U81800 | 603878 | GEN-3WB | Homo sapiens monocarboxylate |

transporter (MCT3) mRNA, complete cds

| | | |
|---|---|---|
| 1485 | 1423C > T | 3′ |
| 1624 | 1562G > C | 3′ |

| | | | | |
|---|---|---|---|---|
| X82321 | X82321 | 600538 | GEN-3WT | H. sapiens mRNA for thiol- |

specific antioxidant

| | | |
|---|---|---|
| 304 | 304G > A | G102R |
| 422 | 422G > T | W141L |
| 640 | 640C > G | 3′ |
| 655 | 655C > T | 3′ |

| | | | | |
|---|---|---|---|---|
| U83411 | U83411 | 603105 | GEN-3Y1 | Homo sapiens carboxypeptidase Z |

precursor, mRNA, complete cds

| | | |
|---|---|---|
| 1683 | 1644C > A | Silent |
| 1788 | 1749G > A | Silent |
| 2007 | 1968A > G | 3′ |
| 2013 | 1974G > A | 3′ |

| | | | | |
|---|---|---|---|---|
| ARSE | X83573 | 300180 | GEN-3Y8 | Homo sapiens APSE gene, complete |
| CDS | | | | |

| | | |
|---|---|---|
| 1759 | 1692C > T | Silent |
| 1795 | 1728G > A | Silent |

| | | | | |
|---|---|---|---|---|
| AF085690 | AF085690 | None | GEN-3YE | Homo sapiens multidrug |

resistance-associated protein 3 (MRP3) mRNA, complete cds

| | | | | |
|---|---|---|---|---|
| 3978C | 4064T | 4386C | 4545A | |
| 3926G | 3978T | 4064C | 4386C | 4545A |
| 3926G | 3978T | 4064C | 4386C | 4545G |
| 3926A | 3978C | 4064C | 4386C | 4545A |
| 3926G | 3978C | 4064C | 4386C | 4545A |
| 3926G | 4064C | 4386T | 4545A | |
| 3926G | 3978C | 4064C | 4386C | 4545G |
| 3926G | 3978C | 4064C | 4386T | 4545A |
| 3978C | 4064T | 4386C | 4545A | |
| 3926G | 3978T | 4064C | 4386C | |

| | | |
|---|---|---|
| 3926 | 3890G > A | R1297H |
| 3978 | 3942C > T | Silent |
| 4064 | 4028C > T | A1343V |
| 4386 | 4350C > T | Silent |
| 4545 | 4509A > G | Silent |
| 5119 | 5083A > C | 3′ |

| | | | | |
|---|---|---|---|---|
| TGFBR2 | M85079 | 190182 | GEN-3ZS | Human TGF-beta type II receptor |

mRNA, complete cds

| | | |
|---|---|---|
| 1334 | 999A > G | Silent |
| 2045 | 1710A > C | 3′ |

| | | | | |
|---|---|---|---|---|
| PXMP3 | M85038 | 170993 | GEN-3ZU | Human 35kD peroxisomal membrane |

protein mRNA, complete cds

| | | |
|---|---|---|
| 102 | (−164)A > C | 5′ |

| | | | | |
|---|---|---|---|---|
| CEL | M85201 | 114841 | GEN-404 | Human cholesterol esterase mRNA, |

complete cds

| | | |
|---|---|---|
| 566 | 558T > C | Silent |
| 1306 | 1298G > A | S433N |
| 1826 | 1818C > T | Silent |

| | | | | |
|---|---|---|---|---|
| YWHAZ | M86400 | 601288 | GEN-40Y | Human phospholipase A2 mRNA, |

complete cds

| | | |
|---|---|---|
| 1653 | 1569T > A | 3′ |
| 2599 | 2515C > G | 3′ |
| 2619 | 2535A > C | 3′ |
| 2656 | 2572A > C | 3′ |
| 2745 | 2661C > T | 3′ |
| 2761 | 2677A > C | 3′ |

| | | | | |
|---|---|---|---|---|
| X86681 | X86681 | 602110 | GEN-41E | H. sapiens mRNA for nucleolar |

protein, HNP36

| | | | | |
|---|---|---|---|---|
| 1537C | 1645T | 1796G | | |
| 1537C | 1645C | 1725G | 1796G | 1915A |
| 1537C | 1645C | 1725G | 1796A | 1915A |
| 1537T | 1645C | 1796G | | |
| 1537C | 1645C | 1725A | 1796G | 1915C |
| 1537T | 1645C | 1725G | 1796G | 1915A |
| 1537C | 1645T | 1725G | 1796G | 1915A |

| | | |
|---|---|---|
| 1537 | 1152C > T | 3′ |
| 1645 | 1260C > T | 3′ |
| 1725 | 1340G > A | 3′ |
| 1796 | 1411G > A | 3′ |
| 1915 | 1530A > C | 3′ |

| | | | | |
|---|---|---|---|---|
| D87292 | D87292 | 180370 | GEN-42Y | Human mRNA for rhodanese, |

complete cds

| | | |
|---|---|---|
| 816 | 768C > T | Silent |
| 946 | 898G > A | 3′ |

| | | | | |
|---|---|---|---|---|
| D87845 | D87845 | 602344 | GEN-44C | Human mRNA for platelet- |

activating factor acetylhydrolase 2, complete cds

| | | |
|---|---|---|
| 2299 | 2096G > A | 3′ |
| 2332 | 2129A > G | 3′ |

| | | | | |
|---|---|---|---|---|
| D88308 | D88308 | 603247 | GEN-44Z | Homo sapiens mRNA for very-long |

chain acyl-CoA synthetase, complete cds

| | | |
|---|---|---|
| 498 | 276C > T | Silent |

| | | | | |
|---|---|---|---|---|
| D90041 | D90041 | 108345 | GEN-464 | Human liver arylamine N- |

acetyltransferase (EC 2.3.1.5) gene

| | | |
|---|---|---|
| 591 | 445G > A | V149I |
| 1240 | 1094C > A | 3′ |

| | | | | |
|---|---|---|---|---|
| AAC2 | D90040 | 243400 | GEN-465 | Human mRNA for arylamine N- |

acetyltransferase (EC 2.3.1.5)

| | | |
|---|---|---|
| 232 | 191G > A | R64Q |
| 323 | 282C > T | Silent |
| 844 | 803A > G | K268R |

| | | | | |
|---|---|---|---|---|
| M90656 | M90656 | 230450 | GEN-46P | Human gamma-glutamylcysteine |

synthetase (GCS) mRNA, complete cds

| | | |
|---|---|---|
| 620 | 528A > G | Silent |

| | | | | |
|---|---|---|---|---|
| X90999 | X90999 | 138760 | GEN-477 | H. sapiens mRNA for Glyoxalase II |

| | | |
|---|---|---|
| 950 | 914A > G | 3′ |

| | | | | |
|---|---|---|---|---|
| U91521 | U91521 | 601758 | GEN-47E | Human peroxin 12 (HsPEX12) mRNA, |

complete cds

| | | |
|---|---|---|
| 1747 | 1597A > G | 3′ |
| 2066 | 1916G > C | 3′ |

| | | | | |
|---|---|---|---|---|
| X92106 | X92106 | 602403 | GEN-47S | H. sapiens mRNA for bleomycin |
| hydrolase | | | | |

| | | |
|---|---|---|
| 681C | 1405A | 1576T |
| 681G | 1405G | 1576C |
| 681G | 1405A | 1576C |
| 681C | 1405G | 1576C |
| 681C | 1405A | 1576C |

| | | |
|---|---|---|
| 681 | 603C > G | Silent |
| 1405 | 1327A > G | I443V |
| 1576 | 1498C > T | 3′ |

| | | | | |
|---|---|---|---|---|
| U92314 | U92314 | 604125 | GEN-47U | Homo sapiens hydroxysteroid |

sulfotransferase SULT2B1a (HSST2) mRNA, complete cds

| | | |
|---|---|---|
| 1146 | 771C > T | Silent |
| 1164 | 789C > T | Silent |
| 1278 | 903T > C | Silent |

| | | | | |
|---|---|---|---|---|
| PNLIP | M93285 | 246600 | GEN-48N | Pancreatic lipase (PNLIP) |

(Dietary supplement)

| | | |
|---|---|---|
| 646 | 646G > T | V216L |

| | | | | |
|---|---|---|---|---|
| X95190 | X95190 | 601641 | GEN-49Y | H. sapiens mRNA for Branched |

chain Acyl-CoA Oxidase

| | | |
|---|---|---|
| 1394 | 1302C > T | Silent |
| 1934 | 1842C > A Silent | |

| | | | | |
|---|---|---|---|---|
| X96395 | X96395 | 601107 | GEN-4AM | H. sapiens mRNA for canalicular |

multidrug resistance protein

| | | | | | |
|---|---|---|---|---|---|
| 1286G | 2971G | 3144T | 4525T | 4564C | 4581G |
| 2971G | 3144T | 4564T | | | |
| 1286G | 2971A | 3144T | 4525C | 4564C | 4581G |
| 1286A | 2971G | 3144T | 4525C | 4564C | 4581G |
| 1286G | 2971G | 3144T | 4525C | 4564C | 4581G |
| 1286G | 2971G | 3144C | 4525C | 4564C | 4581G |
| 1286G | 2971G | 3144T | 4525C | 4564C | 4581A |
| 2971G | 3144T | 4525C | 4564C | 4581G | |
| 1286A | 2971G | 3144T | 4525T | 4564T | 4581A |

| | | |
|---|---|---|
| 848 | 811C > T | A271S |
| 1286 | 1249G > A | V417I |
| 2971 | 2934G > A | Silent |
| 3144 | 3107T > C | Il036T |
| 4525 | 4488C > T | Silent |
| 4564 | 4527C > T | Silent |
| 4581 | 4544G > A | C1515Y |

| | | | | |
|---|---|---|---|---|
| ABC3 | X97l87 | 601615 | GEN-4BI | H. sapiens mRNA for ABC-C |
| transporter | | | | |

| | | |
|---|---|---|
| 4671 | 4324G > T | V1442F |
| 5075 | 4728G > A | Silent |

| | | | | |
|---|---|---|---|---|
| ID2 | M97796 | 600386 | GEN-4C3 | Human helix-loop-helix protein |

(Id-2) mRNA, complete cds

| | | |
|---|---|---|
| 402 | 294C > G | Silent |

| | | | | |
|---|---|---|---|---|
| M98045 | M98045 | 136510 | GEN-4C3 | Homo sapiens folylpolyglutamate |

synthetase mRNA, complete cds

| | | |
|---|---|---|
| 802C | 1747G | 1900T |
| 802T | 1747G | |
| 802C | 1747G | 1900C |

| | | |
|---|---|---|
| 802 | 732C > T | Silent |
| 1747 | 1677G > T | 3′ |
| 1900 | 1830T > C | 3′ |
| 1912 | 1842G > A | 3′ |
| 1995 | 1925C > G | 3′ |

| | | | | |
|---|---|---|---|---|
| L05628 | L05628 | 158343 | GEN-4D9 | Human multidrug resistance- |

associated protein (MRP) mRNA, complete cds

| | | | | | |
|---|---|---|---|---|---|
| 1258C | 1264A | 3369G | 4198G | 4648C | |
| 1258C | 1264G | 3369A | 3976C | 4648C | |
| 1258C | 3369G | 3976C | 4198G | 4648T | |
| 1258T | 1264G | 3369G | 3976C | 4198G | 4648C |
| 1258C | 1264G | 3369G | 3976C | 4198G | 4648C |
| 1258T | 1264G | 3369G | 3976C | 4198A | 4648C |
| 1258C | 1264G | 3369G | 3976C | 4198A | 4648C |
| 1258T | 1264G | 3369G | 3976C | 4198A | |
| 1258C | 1264A | 3369G | 3976T | 4198G | 4648C |
| 1258C | 1264A | 3369G | 3976C | 4198G | 4648T |
| 3369G | 3976C | 4198G | 4648T | | |
| 1258C | 1264G | 3369A | 3976C | 4198A | 4648C |

| | | |
|---|---|---|
| 1258 | 1062T > C | Silent |
| 1264 | 1068G > A | Silent |
| 3369 | 3173G > A | R1058Q |
| 3976 | 3780C > T | Silent |
| 4198 | 4002G > A | Silent |
| 4648 | 4452C > T | Silent |

| | | | | |
|---|---|---|---|---|
| Z34897 | Z34897 | 600167 | GEN-4DE | H. sapiens mRNA for H1 histamine |
| receptor | | | | |

| | | | | |
|---|---|---|---|---|
| 1068A | 1087T | 1135G | 1139T | 1249T |
| 1068A | 1087T | 1135G | 1139C | 1249T |
| 1087C | 1135G | | | |
| 1068A | 1087T | 1135A | 1139C | 1249T |
| 1068A | 1087T | 1135G | 1139C | 1249A |
| 1068G | 1087T | 1135G | 1139C | 1249T |
| 1068A | 1139C | 1249T | | |
| 10680 | 1087C | 1135G | | |

| | | |
|---|---|---|
| 1068 | 1068A > G | Silent |
| 1087 | 1087T > C | S363P |
| 1135 | 1135G > A | G379R |
| 1139 | 1139C > T | S380F |
| 1249 | 1249T > A | L417M |

| | | | | |
|---|---|---|---|---|
| PTGIR | D38128 | 600022 | GEN-4DH | Human IP gene for prostacyclin |

receptor, exon 3

| | |
|---|---|
| 203C | 231A |
| 203C | 231C |
| 203G | 231C |

| | | |
|---|---|---|
| 203 | 203C > G | 3′ |
| 231 | 231C > A | 3′ |

| | | | | |
|---|---|---|---|---|
| M64799 | M64799 | 162020 | GEN-4DN | Histamine receptor H2 |

| | | |
|---|---|---|
| 543 | 543G > A | Silent |

| | | | | |
|---|---|---|---|---|
| L11931 | L11931 | 182144 | GEN-4DT | Human cytosolic serine |

hydroxymethyltransferase (SHMT) mRNA, complete cds

| | | |
|---|---|---|
| 1444C | 1523C | 1541C |
| 1444C | 1523G | 1541T |
| 1444T | 1523C | 1541T |
| 1444C | 1523C | 1541T |
| 1444T | 1523G | 1541T |
| 1444T | 1523C | 1541C |
| 1444T | 1523G | 1541C |
| 1444T | 1541C | |
| 1444C | 1541C | |
| 1444T | 1541T | |

| | | |
|---|---|---|
| 1444 | 1420C > T | L474F |
| 1523 | 1499C > G | 3′ |
| 1541 | 1517C > T | 3′ |

| | | | | |
|---|---|---|---|---|
| L22647 | L22647 | 176802 | GEN-4DZ | Human prostaglandin receptor ep1 |

subtype mRNA, complete cds

| | | |
|---|---|---|
| 841 | 767A > G | H256R |

| | | | | |
|---|---|---|---|---|
| LTC4S | U11552 | 246530 | GEN-4E1 | Human leukotriene-C4 synthase |

mRNA, complete cds

| | | |
|---|---|---|
| 468 | 382G > A | A128T |

| | | | | |
|---|---|---|---|---|
| ATP1A1 | D00099 | 182310 | GEN-4E8 | Homo sapiens mRNA for Na,K- |

ATPase alpha-subunit, complete cds

| | | | | | | |
|---|---|---|---|---|---|---|
| 1059A | 1428G | 2538T | 3324C | 3375G | 3397A | 3408C |
| 1059A | 1428A | 2538T | 3324C | 3375G | 3397G | |
| 1059A | 1428G | 2538T | 3324C | 3375G | 3397G | 3408C |
| 1428G | 2538T | 3324C | 3375A | 3397G | 3408C | |
| 1059C | 1428G | 2538T | 3324T | 3397G | 3408C | |
| 1059A | 1428G | 2538C | 3324C | 3375G | 3397G | 3408C |
| 1059C | 1428G | 2538T | 3324C | 3375G | 3397G | 3408C |
| 1059C | 1428G | 2538T | 3324C | 3375A | 3397G | 3408C |
| 1059A | 1428A | 2538T | 3324C | 3375G | 3397G | 3408A |
| 1059C | 1428A | 2538T | 3324C | 3375G | 3397G | 3408C |

| | | |
|---|---|---|
| 1059 | 741A > C | Silent |
| 1428 | 1110G > A | Silent |
| 2056 | 1738A > G | I580V |
| 2538 | 2220T > C | Silent |
| 3324 | 3006C > T | Silent |
| 3375 | 3057G > A | Silent |
| 3397 | 3079G > A | 3′ |
| 3408 | 3090C > A | 3′ |
| 3505 | 3187C > A | 3′ |
| 3538 | 3220G > T | 3′ |

| | | | | |
|---|---|---|---|---|
| DHFR | J00140 | 126060 | GEN-4E9 | Human dihydrofolate reductase |
| gene | | | | |

| | | | |
|---|---|---|---|
| 666T | 721A | 729T | 829C |
| 666A | 829C | | |
| 666T | 721T | 729T | 829C |
| 666T | 721A | 729T | 829T |
| 666A | 721T | 729C | 829C |
| 721T | 829T | | |

| | | |
|---|---|---|
| 666 | 624T > A | 3′ |
| 721 | 679T > A | 3′ |
| 729 | 687T > C | 3′ |
| 829 | 787C > T | 3′ |

| | | | | |
|---|---|---|---|---|
| HTR1E | M91467 | 182132 | GEN-4EE | Serotonin 5-HT receptors 5-HT1E |

| | | |
|---|---|---|
| 964G | 1097C | 1188G |
| 964A | 1097C | 1188G |
| 964A | 1097C | 1188A |
| 964A | 1097T | 1188G |

| | | |
|---|---|---|
| 964 | 398A > G | K133R |
| 1097 | 531C > T | Silent |
| 1188 | 622G > A | A208T |

| | | | | |
|---|---|---|---|---|
| L05597 | L05597 | 182134 | GEN-4EV | Serotonin 5-HT receptors 5-HT1F |

| | | |
|---|---|---|
| 824 | 600T > C | Silent |
| 1010 786\hat{ }787insAATAAAATTCAT [H262Q;262\hat{ }263insIKFI] | | |

| | | | | |
|---|---|---|---|---|
| SLC18A3 | U09210 | 600336 | GEN-4F3 | Human vesicular acetylcholine |

transporter mRNA, complete cds

| | | | | | | |
|---|---|---|---|---|---|---|
| 838T | 1057G | 1369A | 1567C | 2080G | 2199G | 2349G |
| 1269G | 2080G | 2349G | | | | |
| 838C | 1057G | 1369A | 1567C | 2080G | 2199G | 2349G |
| 1057G | 1369A | 1567C | 2199G | 2349T | | |
| 838T | 1057G | 1369A | 1567C | 2080T | 2199G | 2349G |
| 838C | 1057G | 1369A | 1567C | 2080T | 2199G | 2349T |
| 838C | 1057C | 1369G | 1567G | 2080G | 2199A | 2349G |

| | | |
|---|---|---|
| 838 | 396T > C | Silent |
| 1057 | 615G > C | Silent |
| 1369 | 927A > G | Silent |
| 1567 | 1125C > G | Silent |
| 2080 | 1638G > T | 3′ |
| 2199 | 1757G > A | 3′ |
| 2349 | 1907G > T | 3′ |

| | | | | |
|---|---|---|---|---|
| U09806 | U09806 | 236250 | GBN-4FZ | Human methylenetetrahydrofolate |

reductase mRNA, partial cds

| | | | | | | | |
|---|---|---|---|---|---|---|---|
| 120C | 519C | 668C | 1059T | 1308T | | | |
| 120C | 464T | 519C | 1059C | 1308T | 1784A | | |
| 120C | 464T | 519T | 668C | 1059C | 1289A | 1308C | 1784G |
| 120C | 464T | 519C | 668C | 1059C | 1289A | 1308C | 1784G |
| 120C | 464T | 519T | 1059C | 1289A | 1308T | 1784G | |
| 120T | 464T | 519C | 668C | 1308T | 1784G | | |
| 120C | 464T | 668T | 1059C | 1289C | 1308T | 1784G | |
| 120C | 464T | 519C | 668T | 1059C | 1289A | 1308T | 1784G |
| 120C | 464T | 519C | 668C | 1059C | 1289C | 1308T | 1784G |
| 120C | 464T | 519C | 668C | 1059C | 1289A | 1308T | 1784G |
| 120T | 464T | 519C | 668C | 1059T | 1289C | 1308T | 1784G |
| 120C | 464G | 519C | 668C | 1059T | 1289C | 1308T | 1784A |
| 120T | 464T | 519C | 668C | 1059T | 1289C | 1308T | 1784A |
| 120C | 464T | 519T | 668T | 1059C | 1289C | 1308T | 1784G |
| 120T | 464T | 519T | 668C | 1059T | 1289C | 1308T | 1784G |
| 120C | 668C | 1059C | 1289C | 1308T | 1784G | | |
| 120C | 464T | 519T | 668C | 1059C | 1289A | 1308T | 1784G |
| 120C | 464T | 519C | 668C | 1059C | 1289C | 1308T | 1784A |
| 120C | 464T | 5l9T | 668C | 1059C | 1784G | | |

| | | |
|---|---|---|
| 120 | 120T > C | Silent |
| 464 | 464T > G | M155R |
| 519 | 519C > T | Silent |
| 668 | 668C > T | A223V |
| 1059 | 1059T > C | Silent |
| 1289 | 1289C > A | 3′ |
| 1308 | 1308T > C | 3′ |
| 1784 | 1784G > A | 3′ |

| | | | | |
|---|---|---|---|---|
| U08989 | U08989 | 133550 | GEN-CBZ | Human glutamate transporter |

mRNA, complete cds

| | | |
|---|---|---|
| 684 | 519C > T | Silent |
| 1617 | 1452T > C | Silent |

| | | | | |
|---|---|---|---|---|
| CYP11B2 | D13752 | 124080 | GEN-CCD | Human CYP11B2 gene for steroid |

18-hydroxylase, complete cds

| | | |
|---|---|---|
| 1600 | 1593G > A | 3′ |

| | | | | |
|---|---|---|---|---|
| AB004854 | AB004854 | 603608 | GEN-KV6 | Homo sapiens mRNA for carbonyl |

reductase 3, complete cds

| | | |
|---|---|---|
| 730 | 730G > A | V244M |

| | | | | |
|---|---|---|---|---|
| AJ005162 | AJ005162 | 600067 | GEN-KVT | Homo sapiens mRNA for UDP- |

glucuronosyltransferase

| | |
|---|---|
| 519G | 1845T |
| 519A | 1845T |
| 519G | 1845C |

| | | |
|---|---|---|
| 519 | 486G > A | Silent |
| 1845 | 1812T > C | 3′ |
| 1915 | 1882A > C | 3′ |

| | | | | |
|---|---|---|---|---|
| AB005289 | AB005289 | 300135 | GEN-KVU | Homo sapiens mRNA for ABC |

transporter 7 protein,complete cds

| | | |
|---|---|---|
| 2137 | 2069A > T | H690L |

| | | | | |
|---|---|---|---|---|
| L02932 | L02932 | 170998 | GEN-KW4 | Human peroxisome proliferator |

activated receptor mRNA, complete cds

| | | |
|---|---|---|
| 207 | (−10)T > C | 5′ |
| 648 | 432G > A | Silent |

| | | | | |
|---|---|---|---|---|
| J04132 | J04132 | 186780 | GEN-KXY | Human T cell receptor zeta-chain |

mRNA, complete cds

| | | |
|---|---|---|
| 1403 | 1329G > C | 3′ |
| 1410 | 1336A > T | 3′ |

| | | | | |
|---|---|---|---|---|
| AJ000730 | AJ000730 | 603752 | GEN-KY4 | Homo sapiens mRNA for cationic |

amino acid transporter 3

| | | |
|---|---|---|
| 1126G | 2021T | 2051G |
| 1126A | 2021C | 2051G |
| 1126A | 2021T | 2051A |
| 1126A | 2021T | 2051G |
| 1126G | 2021C | 2051A |
| 1126A | 2021C | 2051A |

| | | |
|---|---|---|
| 195 | 117G > A | Silent |
| 1126 | 1048G > A | A350T |
| 2021 | 1943C > T | 3′ |
| 2051 | 1973A > G | 3′ |

| | | | | |
|---|---|---|---|---|
| L05148 | L05148 | 176947 | GEN-KYC | Human protein tyrosine kinase |

related mRNA sequence

| | | |
|---|---|---|
| 1886 | 1886G > A | 3′ |

| | | | | |
|---|---|---|---|---|
| AB001325 | AB001325 | 600170 | GEN-KYP | Human AQP3 gene for aquaporine 3 |

(water channel), partail cds

| | | |
|---|---|---|
| 1203 | 1143G > A | 3′ |

| | | | | |
|---|---|---|---|---|
| Y08639 | Y08639 | 601972 | GEN-KZ7 | H. sapiens mRNA for nuclear |

orphan receptor ROR-beta

| | |
|---|---|
| 126C | 846A |
| 126T | 846G |
| 126C | 846G |

| | | |
|---|---|---|
| 126 | (−469)C > T | 5′ |
| 846 | 252G > A | Silent |

| | | | | |
|---|---|---|---|---|
| AB014679 | AB014679 | 603798 | GEN-L22 | Homo sapiens GN6ST mRNA for N- |

acetylglucosamine-6-O-sulfotransferase (GlcNAc6ST), complete cds

| | | |
|---|---|---|
| 1578 | 1189G > T | V397L |
| 2335 | 1946T > C | 3′ |

| | | | | |
|---|---|---|---|---|
| AB015050 | AB015050 | 603377 | GEN-L2D | Homo sapiens mRNA for OCTN2, |

complete cds

| | | |
|---|---|---|
| 1101 | 978G > A | Silent |

| | | | | |
|---|---|---|---|---|
| AB017546 | AB017546 | 601791 | GEN-L3J | Homo sapiens Pex14 mPNA for |

peroxisomal membrane anchor protein, complete cds

| | | |
|---|---|---|
| 104 | 99G > A | Silent |

| | | | | |
|---|---|---|---|---|
| AF012390 | AF011390 | 603345 | GEN-L59 | Homo sapiens pancreas sodium |

bicarbonate cotransporter mRNA, complete cds

| | | | | | | | |
|---|---|---|---|---|---|---|---|
| 1131C | 3108T | 3434A | 3647T | 3767C | 4294G | 4345A47330 | |
| 1131C | 3108C | 3434A | 3647T | 3767C | 4345T | 4733G | |
| 1131C | 3434A | 3767G | 4294G | 4345T | 4733G | | |
| 3434C | 3647T | 3767C | 4345T | 4733C | | | |
| 1131C | 3108T | 3434A | 3647T | 3767C | 4294G | 4345T | 4733C |
| 1131C | 3108T | 3434A | 3647T | 3767C | 4294G | 4345T | 4733G |
| 1131T | 3108T | 3647T | 3767C | 4345T | 4733G | | |
| 2131C | 3647C | 3767C | 4345T | 4733C | | | |
| 1131C | 3108T | 3434C | 3647T | 3767C | 4345T | 4733G | |
| 1131C | 3108T | 3434A | 3647T | 3767C | 4294C | 4345T | 4733G |
| 3108C | 4294G | | | | | | |
| 1131C | 3108C | 3434A | 3647C | 3767C | 4294G | 4345T | 4733C |
| 1131T | 3108T | 3434C | 3647T | 3767C | 4294G | 4345T | 4733G |
| 1131C | 3108C | 3434A | 3647C | 3767G | 4294C | 4345T | 4733G |
| 1131C | 3108T | 3434A | 3647T | 3767C | 4345T | 4733G | |
| 1131C | 3108T | 3434C | 3647C | 3767G | 4294C | 4733G | |
| 1131T | 3108C | 3434A | 3647T | 3767C | 4294G | 4345T | 4733C |
| 2131C | 3108T | 3434C | 3647T | 3767C | 4294C | | |
| 1131C | 3108T | 3434C | 3647T | 3767C | 4294G | 4345T | 4733G |
| 1131C | 3108T | 3434C | 3647T | 3767C | 4294C | 4345T | 4733G |
| 1131C | 3108C | 3434A | 3647C | 3767G | 4294G | 4345T | 4733G |

| | | |
|---|---|---|
| 1131 | 1014C > T | Silent |
| 3108 | 2991T > C | Silent |
| 3434 | 3317A > C | 3′ |
| 3647 | 3530T > C | 3′ |
| 3767 | 3650C > G | 3′ |
| 4294 | 4177G > C | 3′ |
| 4345 | 4228T > A | 3′ |
| 4733 | 4616G > C | 3′ |

| | | | | |
|---|---|---|---|---|
| U16997 | U16997 | 602943 | GEN-L5O | Human orphan receptor ROR gamma |

mRNA, complete cds

| | | |
|---|---|---|
| 1545 | 1476C > G | I492M |

| | | | | |
|---|---|---|---|---|
| U21943 | U21943 | 602883 | GEN-L97 | Human organic anion transporting |

polypeptide (OATP) mRNA, complete cds

| | | | |
|---|---|---|---|
| 1964A | 2183A | 2229A | |
| 1964A | 2183A | 2229G | 2295A |
| 1964T | 2183C | 2229G | 2295A |
| 1964A | 2183C | 2229G | 2295C |
| 1964A | 2183C | 2229G | 2295A |
| 1964A | 2183A | 2229A | 2295C |
| 1964A | 2183A | 2229G | 2295C |

| | | |
|---|---|---|
| 1964 | 1911A > T | Silent |
| 2183 | 2130C > A | 3′ |
| 2229 | 2176G > A | 3′ |
| 2295 | 2242A > C | 3′ |

| | | | | |
|---|---|---|---|---|
| AJ225089 | AJ225089 | 603281 | GEN-L99 | Homo sapiens mRNA for 2′-5′ |

oligoadenylate synthetase 59 kDa isoform

| | | |
|---|---|---|
| 1724 | 1718C > T | 3′ |
| 1738 | 1732G > T | 3′ |

| | | | | |
|---|---|---|---|---|
| D26480 | D26480 | None | GEN-LBX | Human mRNA for leukotriene B4 |

omega-hydroxylase, complete cds

| | | | | | | | | | | | |
|---|---|---|---|---|---|---|---|---|---|---|---|
| 75T | 320A | 847C | 872T | 1085G | 1115A | 1121G | 1275C | 1596C | 1780A | 1795A | 2020G |
| 2043G | 2147G | | | | | | | | | | |
| 75T | 320A | 847A | 872T | 1115G | 1121G | 1275C | 1596C | 1795A | 2020G | 2043G | 2147C |
| 320A | 847C | 1115G | 1121G | 1275G | 1596C | 1780G | 1795G | 2020G | 2043G | 2147G | |
| 75T | 320A | 847C | 872T | 1085A | 1115G | 1121G | 1275C | 1780G | 1795A | 2043G | 2147G |
| 75T | 320A | 847C | 872C | 1085G | 1115A | 1121G | 1275C | 1596C | 1780A | 1795A | 2020G |
| 2043G | 2147G | | | | | | | | | | |
| 75T | 320A | 847C | 1115G | 1121G | 1275C | 1596C | 1780A | 1795A | 2020G | 2043G | 2147C |
| 75T | 320A | 847C | 1085G | 1115G | 1121G | 1275C | 1596C | 1780G | 1795A | 2020G | 2043G |
| 2147C | | | | | | | | | | | |
| 75T | 320A | 847C | 1115A | 1121G | 1275G | 1596C | 1780A | 1795A | 2020G | 2043G | 2147C |
| 75T | 320A | 847A | 1085G | 1115A | 1121G | 1275C | 1596C | 1780A | 1795A | 2020G | 2043G |
| 2147G | | | | | | | | | | | |
| 75T | 320A | 847C | 1115G | 1121G | 1275G | 1596C | 1780A | 1795A | 2020G | 2043G | 2147G |
| 75T | 320A | 847C | 1085G | 1115G | 1121G | 1275C | 1596C | 1780G | 1795A | 2020G | 2043G |
| 2147G | | | | | | | | | | | |
| 75T | 320A | 847A | 872C | 1085G | 1121G | 1275C | 1596C | 1795A | 2020G | 2043G | 2147C |
| 75T | 320A | 847C | 872C | 1085A | 1115G | 1121G | 1275C | 1780G | 1795A | 2043G | 2147G |
| 75G | 320A | 847C | 1085A | 1115G | 1121G | 1275C | 1596C | 1780G | 1795A | 2020G | 2043G |
| 75T | 320A | 847C | 1085G | 1115A | 1121G | 1275C | 1596C | 1780G | 1795A | 2020G | 2043T |
| 2147G | | | | | | | | | | | |
| 75T | 320A | 847C | 1085A | 1115G | 1121G | 1275C | 1596C | 1780G | 1795A | 2020G | 2043G |
| 2147C | | | | | | | | | | | |
| 75G | 847C | 1115G | 1121G | 1275C | 1596C | 1780A | 1795A | 2020G | 2043G | 2147G | |
| 75T | 320A | 872C | 1121G | 1275A | 1596C | 1780G | 1795A | 2020G | 2043G | | |
| 75T | 320A | 847C | 1085G | 1115A | 1121G | 1275C | 1596C | 1780G | 1795A | 2020G | 2043G |
| 2147G | | | | | | | | | | | |
| 75T | 320A | 847C | 1115G | 1121A | 1275C | 1596C | 1780A | 1795A | 2020G | 2043G | 2147C |
| 75T | 320A | 847C | 1085G | 1115A | 1121G | 1275C | 1596C | 1780G | 1795A | 2020G | 2043G |
| 2147C | | | | | | | | | | | |
| 75T | 320A | 847C | 872T | 1085A | 1115G | 1121G | 1275C | 1596C | 1780G | 1795A | 2020G |
| 2043G | 2147C | | | | | | | | | | |
| 75T | 320A | 847A | 872C | 1085G | 1115A | 1121G | 1275C | 1596C | 1780A | 1795A | 2020G |
| 2043G | 2147C | | | | | | | | | | |
| 75T | 320A | 847C | 872C | 1085A | 1115A | 1121G | 1275C | 1596C | 1780A | 1795A | 2020G |
| 2043G | 2147C | | | | | | | | | | |
| 75T | 320A | 847A | 872C | 1085G | 1115A | 1121G | 1275C | 1596C | 1780A | 1795A | 2020G |
| 2043G | 2147G | | | | | | | | | | |
| 75T | 320A | 847A | 872C | 1085G | 1115A | 1121G | 1275A | 1596C | 1780G | 1795A | 2020G |
| 2043G | 2147C | | | | | | | | | | |
| 75T | 320A | 847C | 872T | 1085G | 1115A | 1121G | 1275C | 1596C | 1780G | 1795A | 2020G |
| 2043G | 2147C | | | | | | | | | | |
| 75T | 320A | 847C | 872C | 1085G | 1115A | 1121G | 1275C | 1596C | 1780G | 1795A | 2020G |
| 2043G | 2147C | | | | | | | | | | |
| 75T | 320A | 847C | 872T | 1085A | 1115G | 1121G | 1275C | 1596C | 1780A | 1795A | 2020G |
| 2043G | 2147C | | | | | | | | | | |
| 75T | 320A | 847C | 872T | 1085G | 1115A | 1121G | 1275C | 1596C | 1780G | 1795A | 2020G |
| 2043G | 2147G | | | | | | | | | | |
| 75T | 320A | 847C | 872T | 1085G | 1115G | 1121G | 1275C | 1596C | 1780G | 1795G | 2020G |
| 2043G | 2147G | | | | | | | | | | |
| 75T | 320A | 847C | 872C | 1085A | 1115G | 1121G | 1275C | 1596C | 1780A | 1795A | 2020G |
| 2043G | 2147G | | | | | | | | | | |
| 75T | 320A | 847A | 872C | 1085A | 1115G | 1121G | 1275C | 1596C | 1780G | 1795A | 2020G |
| 2043G | 2147C | | | | | | | | | | |
| 75T | 320A | 847A | 872C | 1085A | 1115G | 1121G | 1275C | 1596C | 1780G | 1795A | 2020G |
| 2043G | 2147G | | | | | | | | | | |
| 75T | 320A | 847C | 872T | 1085A | 1115G | 1121G | 1275C | 1596A | 1780G | 1795A | 2020A |
| 2043G | 2147G | | | | | | | | | | |
| 75T | 320A | 847C | 872T | 1085G | 1115G | 1121G | 1275C | 1596C | 1780G | 1795A | 2020G |
| 2043G | 2147G | | | | | | | | | | |
| 75T | 320A | 847C | 872C | 1085G | 1115G | 1121G | 1275C | 1596C | 1780G | 1795A | 2020G |
| 2043G | 2147C | | | | | | | | | | |
| 75T | 320A | 847C | 872T | 1085A | 1115G | 1121G | 1275C | 1596C | 1780G | 1795A | 2020G |
| 2043G | 2147C | | | | | | | | | | |
| 75T | 320A | 847C | 872C | 1085A | 1115G | 1121A | 1275C | 1596C | 1780A | 1795A | 2020G |
| 2043G | 2147C | | | | | | | | | | |
| 75T | 320A | 847C | 872C | 1085G | 1115A | 1121G | 1275C | 1596C | 1780G | 1795A | 2020G |
| 2043T | 2147G | | | | | | | | | | |
| 75T | 320A | 847C | 872C | 1085G | 1115G | 1121G | 1275C | 1596C | 1780G | 1795A | 2020G |
| 2043G | 2147G | | | | | | | | | | |
| 75T | 320A | 847A | 872T | 1085G | 1115A | 1121G | 1275C | 1596C | 1780G | 1795A | 2020G |
| 2043G | 2147C | | | | | | | | | | |
| 75T | 320A | 847C | 872C | 1085A | 1115G | 1121G | 1275C | 1596C | 1780G | 1795A | 2020G |
| 2043G | 2147G | | | | | | | | | | |
| 75T | 320A | 847A | 872T | 1085G | 1115G | 1121G | 1275C | 1596C | 1780G | 1795A | 2020G |
| 2043G | 2147C | | | | | | | | | | |
| 75T | 320A | 847C | 872T | 1085G | 1115G | 1121G | 1275C | 1596C | 1780A | 1795A | 2020G |
| 2043G | 2147C | | | | | | | | | | |
| 75T | 320A | 847C | 872T | 1085A | 1115A | 1121G | 1275C | 1596C | 1780G | 1795a | 2020G |
| 2043G | 2147C | | | | | | | | | | |
| 75T | 320A | 847C | 872T | 1085G | 1115G | 1121G | 1275C | 1596C | 1780A | 1795A | 2020G |
| 2043G | 2147G | | | | | | | | | | |

| | | |
|---|---|---|
| 75 | 34T > G | W12G |
| 320 | 279A > C | Silent |
| 847 | 806C > A | A269D |
| 872 | 83lT > C | Silent |
| 1085 | 1044A > G | Silent |
| 1115 | 1074G > A | Silent |
| 1121 | 1080A > G | Silent |
| 1275 | 1234C > A | Silent |
| 1439 | 13980 > A | Silent |
| 1596 | l555C > A | L5l9M |
| 1780 | 1739G > A | 3′ |
| 1795 | 1754A > G | 3′ |
| 2003 | 1962C > T | 3′ |
| 2020 | 1979G > A | 3′ |
| 2043 | 2002G > T | 3′ |
| 2147 | 2106C > G | 3′ |

| | | | | |
|---|---|---|---|---|
| AJ130718 | AJ130718 | 603593 | GEN-LDO | Homo sapiens mRNA for |

glycoprotein-associated amino acid transporter y+LAT1

| | | |
|---|---|---|
| 791T | 953T | 1820A |
| 791C | 953C | 1820A |
| 791C | 953C | 1820G |
| 791T | 953C | 1820G |
| 791C | 953T | 1820A |
| 791C | 953T | 1820G |
| 791T | 953T | 1820G |
| 791T | 953C | 1820A |

| | | |
|---|---|---|
| 791 | 498C > T | Silent |
| 953 | 660T > C | Silent |
| 1820 | 1527G > A | Silent |

| | | | | |
|---|---|---|---|---|
| AF031416 | AF031416 | 603258 | GEN-LDU | Homo sapiens IkB kinase beta |

subunit mRNA, complete cds

| | | |
|---|---|---|
| 2028 | 2028G > A | M676I |

| | | | | |
|---|---|---|---|---|
| AF058921 | AF058921 | 603602 | GEN-LJY | Homo sapiens cytosolic |

phospholipase A2-gamma mRNA, complete cds

| | | |
|---|---|---|
| 1972 | 1663G > A | 3′ |
| 1989 | 1680A > T | 3′ |

| | | | | |
|---|---|---|---|---|
| AF060502 | AF060502 | 602859 | GEN-LL7 | Homo sapiens peroxisome assembly |

protein PEX10 mRNA, complete cds

| | | |
|---|---|---|
| 1186 | 1154G > A | 3′ |

| | | | | |
|---|---|---|---|---|
| AF070548 | AF070548 | 604165 | GEN-LNS | Homo sapiens clone 24408 2- |

oxoglutarate carrier protein mRNA, complete cds

| | | |
|---|---|---|
| 1224 | 1113C > T | 3′ |
| 1483 | 1372A > C | 3′ |

| | | | | |
|---|---|---|---|---|
| AF071202 | AF071202 | None | GEN-LP3 | Homo sapiens ABC transporter |

MOAT-B (MOAT-B) mRNA, complete cds

| | | | | | | | | | | |
|---|---|---|---|---|---|---|---|---|---|---|
| 674G | 1027G | 1084A | 1612C | 2384G | 2827G | 2959C | 2962C | 3445T | 3463G | 4131G |
| 674T | 1084G | 1612C | 2384A | 2827G | 2959C | 2962C | 3445T | 3463A | 4131T | |
| 674G | 1027G | 1084A | 1612C | 2384G | 2827G | 2959C | 2962C | 3445T | 3463A | 4131T |
| 674G | 1084A | 1612C | 2384G | 2827A | 2959T | 2962C | 3445T | 3463G | 4131T | |
| 674T | 1027G | 1084G | 1612C | 2384G | 2827G | 2959C | 2962C | 3445T | 3463A | |
| 674G | 1027G | 1084A | 2384G | 2827G | 2959C | 2962C | 3445T | 3463G | 4131T | |
| 674G | 1027G | 1084G | 1612C | 2384G | 2827G | 2959C | 2962C | 3445T | 3463A | 4131G |
| 674T | 1027G | 1084G | 1612C | 2384G | 2959T | 2962C | 3445T | 4131T | | |
| 674G | 1027G | 2384G | 2827G | 2959C | 2962C | 3445C | 3463A | | | |
| 674G | 1027G | 2384A | 2959C | 3445T | | | | | | |
| 674G | 1027G | 1084G | 1612C | 2384G | 2827G | 2959C | 2962C | 3445T | 3463A | 4131T |
| 674G | 1027G | 1084A | 1612C | 2384G | 2827G | 2959C | 2962C | 3445T | 3463A | 4131G |
| 674G | 1027G | 1084G | 2384G | 2827A | 2959C | 3445T | 4131T | | | |
| 674G | 1084G | 1612C | 2384G | 2827A | 2959T | 2962C | 3445T | 3463G | 4131T | |
| 674G | 1027G | 1612T | 2384G | 2827G | 2959C | 2962C | 3445T | 3463A | 4131G | |
| 674G | 1027T | 1612C | 2384G | 2827G | 2959C | 2962C | 3445T | 3463A | | |
| 674G | 1027G | 1084G | 1612C | 2384G | 2827G | 2959C | 2962C | 3445T | 3463G | |
| 674T | 1027G | 1084G | 1612C | 2384G | 2827G | 2959C | 2962C | 3445T | 3463A | 4131G |
| 674G | 1027G | 1084A | 1612T | 2384A | 2827A | 2959C | 2962T | 3445T | 3463G | 4131G |
| 674T | 1027G | 1084G | 1612C | 2384G | 2827G | 2959C | 2962C | 3445T | 3463A | 4131T |
| 674G | 1027G | 1084A | 1612C | 2384G | 2827G | 2959C | 2962C | 3445T | 3463G | 4131T |
| 674G | 1027T | 1612C | 2384G | 2827G | 2959C | 2962C | 3445T | 3463A | 4131G | |
| 674G | 1027G | 1084G | 1612C | 2384G | 2827G | 2959C | 2962C | 3445T | 3463G | 4131G |
| 674G | 1027G | 1084A | 1612T | 2384G | 2827G | 2959C | 2962C | 3445T | 3463A | 4131G |
| 674T | 1027G | 1084G | 1612C | 2384G | 2827A | 2959T | 2962C | 3445T | 3463G | 4131T |
| 674G | 1027G | 1084A | 2384G | 2827G | 2959C | 2962C | 3445T | 3463G | 4131T | |
| 674T | 1027G | 1612C | 2384G | 2827G | 2959C | 2962C | 3445T | 3463A | 4131G | |
| 674G | 1027T | 1084A | 1612C | 2384G | 2827A | 2959T | 2962C | 3445T | 3463G | 4131T |
| 674G | 1027T | 1084G | 1612C | 2384G | 2827A | 2959T | 2962C | 3445T | 3463G | 4131G |
| 674G | 1027G | 1084A | 1612T | 2384G | 2827G | 2959C | 2962C | 3445C | 3463A | 4131G |
| 674G | 1027G | 1084A | 1612C | 2384G | 2827A | 2959T | 2962C | 3445T | 3463G | 4131G |
| 674G | 1027T | 1084A | 1612C | 2384G | 2827G | 2959C | 2962C | 3445T | 3463G | 4131T |
| 674G | 1027G | 1084A | 1612T | 2384G | 2827G | 2959C | 2962C | 3445T | 3463G | 4131T |
| 674T | 1027T | 1084G | 1612C | 2384A | 2827G | 2959C | 2962C | 3445T | 3463A | 4131T |
| 674G | 1027T | 1084G | 1612C | 2384G | 2827A | 2959T | 2962C | 3445T | 3463G | 4131T |
| 674G | 1027G | 1084A | 1612T | 2384G | 2827G | 2959C | 2962C | 3445T | 3463A | |
| 674G | 1027G | 1084A | 1612C | 2384G | 2827A | 2959T | 2962C | 3445T | 3463G | 4131T |
| 674G | 1027G | 1084G | 1612T | 2384G | 2827A | 2959C | 2962T | 3445T | 3463G | 4131T |

| | | |
|---|---|---|
| 674 | 559G > T | G187W |
| 1027 | 912G > T | K304N |
| 1084 | 969C > A | Silent |
| 1612 | 1497T > C | Silent |
| 2384 | 2269G > A | E757K |
| 2827 | 2712G > A | Silent |
| 2959 | 2844C > T | Silent |
| 2962 | 2847C > T | Silent |
| 3445 | 3330T > C | Silent |
| 3463 | 3348A > G | Silent |
| 4131 | 4016T > G | 3′ |

| | | | | |
|---|---|---|---|---|
| U79745 | U79745 | 603880 | GEN-LPT | Homo sapiens monocarboxylate |

transporter homologue MCT6 mRNA, complete cds

| | | |
|---|---|---|
| 775T | 816A | 1476G |
| 775A | 816A | 1476G |
| 775T | 816C | 1476G |
| 775A | 816C | 1476G |
| 775A | 816C | 1476A |

| | | |
|---|---|---|
| 775 | 610A > T | I204F |
| 816 | 651A > C | E217D |
| 1476 | 1311G > A | Silent |
| 2095 | 1930G > A | 3′ |

| | | | | |
|---|---|---|---|---|
| D89053 | D89053 | 602371 | GEN-LRT | Homo sapiens mRNA for Acyl-CoA |

synthetase 3, complete cds

| | |
|---|---|
| 2035A | 2432G |
| 2035C | 2432A |
| 2035A | 2432A |

| | | |
|---|---|---|
| 2035 | 1893A > C | E631D |
| 2432 | 2290A > G | 3′ |

| | | | | |
|---|---|---|---|---|
| X90908 | X90908 | 600422 | GEN-LSA | H. sapiens mRNA for I-15P (I- |

BABP) protein

| | | |
|---|---|---|
| 364 | 236C > T | T79M |

| | | | | |
|---|---|---|---|---|
| X97868 | X97868 | 300003 | GEN-LTH | H. sapiens mRNA for |

arylsulphatase

| | | |
|---|---|---|
| 1652 | 1582T > C | Y528H |

| | | | | |
|---|---|---|---|---|
| AF093771 | AF093771 | 603756 | GEN-LTJ | Homo sapiens mitoxantrone |

resistance protein 1 mRNA, partial sequence

| | | |
|---|---|---|
| 528 | 528G > A | 3′ |

| | | | | |
|---|---|---|---|---|
| X98333 | X98333 | 602608 | GEN-LTN | H. sapiens mRNA for organic |

cation transporter, kidney

| | | |
|---|---|---|
| 201 | 57G > A | Silent |

| | | | | |
|---|---|---|---|---|
| NM_005094 | NM_005094 | 600691 | GEN-LU3 | Homo sapiens fatty acid |

transport protein 4 (FATP4) mRNA

| | |
|---|---|
| 168C | 591A |
| 168T | 591G |
| 168C | 591G |

| | | |
|---|---|---|
| 168 | 168C > T | Silent |
| 591 | 591G > A | Silent |

| | | | | |
|---|---|---|---|---|
| U24253 | U24253 | 136510 | GEN-LUE | Human folylpolyglutamate |

synthetase (FPGS) gene, exons 5-11, and partial cds

| | | | |
|---|---|---|---|
| 1424C | 1649A | 1678C | 1912C |
| 1424C | 1649A | 1678C | 1912T |
| 1424C | 1649G | 1678C | 1912C |
| 1424A | 1649G | 1678C | 1912C |
| 1424C | 1649G | 1678T | 1912C |
| 1424A | 1649G | 2554A | |
| 1424C | 1649A | 1678C | 1912T |
| 1424C | 1649G | 1678C | 1912C |
| 1424C | 1649A | 1678C | 1912C |
| 1424C | 1649G | 1678T | 1912C |

| | | |
|---|---|---|
| 1424 | 1424C > A | Genomic |
| 1649 | 1649G > A | Genomic |
| 1678 | 1678C > T | Genomic |
| 1912 | 1912C > T | Genomic |
| 2554 | 2554A > G | Genomic |

| | | | | |
|---|---|---|---|---|
| U24252 | U24252 | 136510 | GEN-LUF | Folylpolyglutamate synthetase, |

promoter and exons 1-4

| | | | | | | | | | | | |
|---|---|---|---|---|---|---|---|---|---|---|---|
| 263A | 266G | 527C | 1139G | 1217C | 1647C | 2017G | 2037G | 2282T | 2309A | | |
| 263A | 266G | 527C | 1037G | 1139G | 1217C | 1647C | 1955A | 2017G | 2037G | 2282C | 2309A |
| 263G | 266T | 527C | 1647C | 1955A | 2017G | 2037G | 2282C | 2309A | | | |
| 263A | 266T | 527C | 1037G | 1139G | 1217C | 1647C | 1955A | 2017A | 2037G | 2282C | 2309A |
| 263A | 527C | 1037G | 1139G | 1217T | 1647C | 1955A | 2017G | 2282C | 2309A | | |
| 263A | 527C | 1037G | 1139G | 1217T | 1647C | 1955A | 2017G | 2282C | 2309G | | |
| 263A | 266T | 527C | 1037G | 1139G | 1217C | 1647C | 1955A | 2017G | 2037G | 2282C | 2309A |
| 266G | 527C | 1037A | 1139G | 1217C | 1647C | 1955A | 2017G | 2189A | 2282C | | |
| 266G | 527C | 1037G | 1139G | 1217C | 1647C | 1955A | 2017A | 2189A | 2282C | | |
| 263A | 266G | 527C | 1037G | 1139A | 1217C | 1955A | 2017G | 2037G | 2282C | 2309A | |
| 263A | 527C | 1139G | 1217C | 1647C | 1955G | 2017A | 2037G | 2282C | 2309A | | |
| 263A | 266G | 527C | 1139G | 1217C | 1647C | 1955G | 2017G | 2037G | 2282C | 2309A | |
| 263A | 266G | 527G | 1037G | 1139G | 1217C | 1647T | 1955A | 2017G | 2037G | 2282C | 2309A |
| 263A | 266G | 527C | 1037G | 1139G | 1217C | 1647C | 1955A | 2017G | 2037A | 2282C | 2309A |
| 263A | 266G | 527C | 1037G | 1139G | 1217C | 1647T | 1955A | 2017G | 2037G | 2282C | 2309A |
| 266T | 527C | 1037G | 1139G | 1217T | 1647C | 1955A | 2017G | 2189G | 2282C | | |
| 266T | 527C | 1037G | 1139G | 1217C | 1647C | 1955A | 2017A | 2189A | 2282C | | |
| 263A | 266G | 527C | 1037A | 1139G | 1217C | 1647C | 1955G | 2017G | 2037G | 2282C | 2309A |
| 266G | 527C | 1037G | 1139G | 1217C | 1647T | 1955A | 2017G | 2189A | 2282C | | |
| 263A | 266T | 527C | 1037G | 1139G | 1217C | 1647C | 1955A | 2017A | 2037G | 2282C | 2309A |
| 266T | 527C | 1037G | 1139G | 1217T | 1647C | 1955A | 2017G | 2189A | 2282C | | |
| 263A | 266G | 1037A | 1139G | 1217C | 1647C | 1955G | 2017G | 2037G | 2282C | 2309A | |
| 266T | 527C | 1037A | 1139G | 1217C | 1647C | 1955G | 2017G | 2189A | 2282C | | |
| 266G | 527C | 1037G | 1139G | 1217C | 1647C | 1955A | 2017A | 2189A | 2282C | | |
| 263A | 266G | 527C | 1037G | 1139G | 1217T | 1647C | 1955A | 2017G | 2037G | 2282C | 2309G |
| 263A | 266G | 527G | 1037G | 1139G | 1217C | 1647T | 1955A | 2017G | 2037G | 2282C | 2309A |
| 266G | 527C | 1037A | 1139G | 1217C | 1647C | 1955A | 2017G | 2189A | 2282C | | |
| 263A | 266G | 527C | 1037G | 1139G | 1217C | 1647T | 1955A | 2017G | 2037G | 2282C | 2309A |
| 263A | 266G | 527C | 1037A | 1139A | 1217C | 1647T | 1955G | 2017G | 2037G | 2282C | 2309A |
| 263A | 266T | 527C | 1037A | 1139G | 1217C | 1647C | 1955G | 2017A | 2037G | 2282C | 2309A |
| 263A | 266G | 527C | 1037G | 1139A | 1217C | 1647T | 1955A | 2017G | 2037G | 2282C | 2309A |
| 266G | 527C | 1037G | 1139A | 1217C | 1647T | 1955A | 2017G | 2189A | 2282C | | |
| 266G | 527C | 1037A | 1139G | 1217C | 1647C | 1955G | 2017G | 2189A | 2282C | | |
| 266G | 527C | 1037A | 1139G | 1217C | 1647C | 1955G | 20170 | 2189A | 2282T | | |
| 263A | 266G | 527C | 1647C | 1955G | 2017G | 2037G | 2282C | 2309A | | | |
| 266G | 527C | 1037G | 1139G | 1217C | 1647C | 1955A | 2017G | 2189G | 2282C | | |
| 266G | 527C | 1037G | 1139G | 1217C | 1647C | 1955A | 2017G | 2189A | 2282C | | |
| 263A | 266T | 527C | 1037A | 1139G | 1217C | 1647C | 1955G | 2017G | 2037G | 2282C | 2309A |
| 266T | 527C | 1037G | 1139G | 1217C | 1647C | 1955A | 2017G | 2189A | 2282C | | |
| 263A | 266G | 527C | 1037A | 1139G | 1217C | 1647C | 1955G | 2017G | 2037G | 2282T | 2309A |
| 266G | 527C | 1037A | 1139A | 1217C | 1647T | 1955G | 2017G | 2189A | 2282C | | |

| | | |
|---|---|---|
| 263 | 263A > G | Genomic |
| 266 | 266G > T | Genomic |
| 527 | 527C > G | Genomic |
| 1037 | 1037A > G | Genomic |
| 1139 | 1139G > A | Genomic |
| 1217 | 1217C > T | Genomic |
| 1647 | 1647C > T | Genomic |
| 1955 | 1955G > A | Genomic |
| 2017 | 2017G > A | Genomic |
| 2037 | 2037G > A | Genomic |
| 2189 | 2189A > G | Genomic |
| 2282 | 2282C > T | Genomic |
| 2309 | 2309A > G | Genomic |

| | | | | |
|---|---|---|---|---|
| U92868 | U92868 | 600424 | GEN-LUK | Homo sapiens reduced folate |

carrier (RFC1) gene, exons 1a, 1c and 1b

| | | | | |
|---|---|---|---|---|
| 441G | 498C | 579G | 599G | |
| 431A | 441A | 498C | 579G | 599G |
| 431A | 441A | 498T | | |
| 431G | 441G | 498C | 579G | 599G |
| 441G | 498C | 579G | 599G | |
| 431A | 441A | 498T | 579C | 599G |

| | | |
|---|---|---|
| 431 | 431A > G | Genomic |
| 441 | 441A > G | Genomic |
| 498 | 498G > T | Genomic |
| 579 | 579G > C | Genomic |
| 599 | 599G > C | Genomic |

| | | | | |
|---|---|---|---|---|
| X96751 | X96751 | 114835 | GEN-LUL | Carboxylesterase I, promoter |

| | | | | |
|---|---|---|---|---|
| 235C | 328T | 975A | 984G | |
| 235T | 328T | 939G | 975A | 984G |
| 235T | 328T | 939T | 975A | 984G |
| 235T | 328C | 939T | 975A | 984G |
| 235T | 328T | 939T | 975G | 984G |
| 235T | 328T | 939T | 975A | 984C |
| 235C | 328C | 939T | 975A | 984G |
| 235T | 328T | 939G | 975G | 984G |
| 235C | 328T | 939G | 975A | 984G |

| | | |
|---|---|---|
| 235 | 235T > C | Genomic |
| 258 | 258\hat{ }insC | Genomic |
| 328 | 328T > C | Genomic |
| 939 | 939G > T | Genomic |
| 975 | 975A > G | Genomic |
| 984 | 984G > C | Genomic |

| | | | | |
|---|---|---|---|---|
| L06484 | L06484 | 100740 | GEN-LUM | Human acetylcholinesterase |

(ACHE) gene, exons 1-2, and promoter region

| | | | | | | | | | |
|---|---|---|---|---|---|---|---|---|---|
| 700C | 748C | 1274G | 1534T | 1609C | 1690G | 1779A | | | |
| 400C | 1274G | 1534C | 1609G | 1690G | 1744A | 1779G | 1803G | | |
| 114A | 400C | 748C | 1274G | 1534C | 1609G | 1690G | 1744C | 1779G | 1803G |
| 700C | 1274C | 1609C | 1690G | 1779G | | | | | |
| 400C | 1274G | 1534C | 1609G | 1690G | 1744C | 1779A | 1803G | | |
| 114C | 400C | 748C | 1274G | 1534T | 1609G | 1690G | 1744C | 1779A | 1803G |
| 114C | 400C | 748C | 1274G | 1534T | 1609G | 1690G | 1744C | 1779A | 1803A |
| 700C | 748C | 1274G | 1534T | 1609C | 1690A | 1779G | | | |
| 700C | 748T | 1274G | 1534T | 1609C | 1690G | 1779G | | | |
| 700C | 748C | 1274G | 1534T | 1609C | 1690G | 1779G | | | |
| 114C | 400T | 1274G | 1534T | 1609G | 1690G | 1744C | 1779A | | |
| 114C | 400T | 748T | 1274G | 1534T | 1609G | 1690G | 1744C | 1779G | 1803G |
| 700T | 748C | 1274G | 1609C | 1690G | | | | | |
| 114C | 400C | 748C | 1274G | 1534C | 1609G | 1690G | 1744C | 1779G | 1803G |
| 114C | 400C | 748C | 1274G | 1534T | 1609G | 1690G | 1744C | 1779G | 1803G |
| 114A | 400C | 748C | 1274G | 1534C | 1609G | 1690A | 1744C | 1779G | 1803G |
| 1274C | 1609G | 1690G | 1744C | 1779G | 1803G | | | | |
| 700C | 748C | 1274G | 1534C | 1609C | 1690G | 1779G | | | |
| 114A | 400C | 748C | 1274G | 1534C | 1609G | 1690G | 1744C | 1779G | 1803G |
| 700C | 748C | 1274G | 1534C | 1609C | 1690G | 1779A | | | |
| 700C | 748T | 1274C | 1534C | 1609C | 1690G | 1779G | | | |
| 114C | 400C | 748C | 1274G | 1534T | 1609G | 1690G | 1744C | 1779G | 1803G |
| 114C | 400C | 1274G | 1534T | 1609G | 1690G | 1744C | 1779A | 1803A | |
| 114C | 400C | 748C | 1274G | 1534C | 1609G | 1690G | 1744C | 1779A | 1803G |
| 700C | 748C | 1274G | 1534T | 1609C | 1690A | 1779G | | | |
| 700C | 748T | 1274G | 1534C | 1609C | 1690G | 1779G | | | |
| 114A | 400C | 748C | 1274G | 1534C | 1609G | 1690G | 1744C | 1779A | 1803G |
| 114C | 400C | 748C | 1274G | 1534C | 1609G | 1690G | 1744C | 1779G | 1803G |
| 114C | 400T | 748T | 1274C | 1534T | 1609G | 1690G | 1744C | 1779G | 1803G |
| 700T | 748C | 1274G | 1534C | 1609C | 1690G | 1779A | | | |
| 748C | 1274G | 1534T | 1609G | 1690G | 1744C | 1779A | 1803A | | |
| 114A | 400C | 748C | 1274G | 1534C | 1609G | 1690A | 1744C | 1779G | 1803G |
| 114C | 400C | 748C | 1274C | 1534T | 1609G | 1690G | 1744C | 1779A | 1803A |
| 400T | 1274G | 1534T | 1609G | 1690G | 1744C | 1779G | 1803G | | |
| 400C | 748C | 1274G | 1534C | 1609G | 1690G | 1744C | 1779A | 1803G | |
| 700C | 748C | 1274G | 1534T | 1609C | 1690G | 1779A | | | |
| 700C | 748C | 1274G | 1534C | 1609G | 1690G | 1779A | | | |
| 400T | 748T | 1274G | 1534T | 1609G | 1690A | 1744C | 1779G | 1803G | |
| 114C | 400C | 748C | 1274G | 1534T | 1609G | 1690G | 1744C | 1779A | 1803G |
| 700C | 748T | 1274G | 1534C | 1609G | 1690A | 1779G | | | |
| 700C | 748T | 1274G | 1534T | 1609G | 1690G | 1779A | | | |
| 114C | 400T | 748T | 1274G | 1534T | 1609G | 1690G | 1744C | 1779A | 1803G |
| 700C | 748T | 1274G | 1534T | 1609G | 1690G | 1779G | | | |
| 700C | 748C | 1274G | 1534T | 1609G | 1690G | 1779G | | | |
| 114C | 400T | 748T | 1274G | 1534T | 1609G | 1690G | 1744C | 1779G | 1803G |
| 114A | 400C | 1274G | 1534C | 1609G | 1690G | 1744C | 1779A | 1803G | |
| 400C | 748C | 1534C | 1609G | 1690G | 1744C | 1779A | 1803G | | |
| 400C | 748C | 1274G | 1534T | 1609G | 1690G | 1744C | 1779A | 1803A | |
| 114C | 400T | 748C | 1274G | 1534T | 1609G | 1690G | 1744C | 1779A | 1803G |
| 1534C | 1690G | 1744A | 1779G | 1803G | | | | | |

| | | |
|---|---|---|
| 114 | 114C > A | Genomic |
| 400 | 400C > T | Genomic |
| 700 | 700C > T | Genomic |
| 748 | 748C > T | Genomic |
| 1274 | 1274G > C | Genomic |
| 1534 | 1534T > C | Genomic |
| 1609 | 1609G > C | Genomic |
| 1690 | 1690G > A | Genomic |
| 1744 | 1744C > A | Genomic |
| 1779 | 1779A > G | Genomic |
| 1803 | 1803A > G | Genomic |

| | | | | |
|---|---|---|---|---|
| L42812 | L42812 | 100740 | GEN-LUN | Homo sapiens |

acetylcholinesterase (ACHE) gene, exons 2-6

| | | | | | | | | |
|---|---|---|---|---|---|---|---|---|
| 261C | 1871C | 2384C | 2710G | 2831G | 3541G | 4047C | | |
| 261C | 1871T | 2309G | 2384C | 2710A | 2831G | 3290C | 3541G | 4047C |
| 261C | 1871C | 2309G | 2384C | 2710A | 2831G | 3290C | 3541G | 4047C |
| 261C | 1871C | 2384C | 2710A | 2831G | 3290C | 3541A | 4047C | |
| 261C | 1871C | 2309A | 2384C | 2710A | 2831A | 3290C | 3541G | 4047A |
| 1871T | 2309A | 2384C | | | | | | |
| 261C | 1871C | 2309A | 2384C | 2710A | 2831G | 3290C | 3541G | 4047C |
| 261T | 1871C | 2309G | 2384C | 2710A | 2831G | 3290C | 3541G | 4047C |
| 261C | 1871C | 2309A | 2384C | 2710A | 2831G | 3290G | 3541G | 4047C |
| 261C | 1871C | 2309G | 2384T | 2710A | 2831G | 3290C | 3541G | 4047C |
| 261C | 1871C | 2309A | 2384C | 2710A | 2831A | 3290C | 3541G | 4047C |
| 261C | 1871C | 2309A | 2384C | 2710G | 2831G | 3290C | 3541G | 4047C |
| 261C | 1871C | 2309A | 2384C | 3290C | 3541G | 4047C | | |
| 1871C | 2384T | | | | | | | |
| 261C | 1871C | 2309A | 2384C | 2710A | 2831G | 3290G | 4047C | |
| 1871C | 2309G | 2384C | | | | | | |
| 261C | 1871C | 2309A | 2384C | 2710G | 2831G | 3290G | 3541G | 4047C |
| 1871T | 2309A | 2384C | | | | | | |
| 261C | 1871T | 2309A | 2384C | 3290C | 3541G | 4047C | | |
| 261C | 1871T | 2309A | 2384C | 2710A | 2831G | 3290C | 3541G | 4047C |
| 261C | 1871C | 2309A | 2384C | 2710A | 2831G | 3290C | 3541A | 4047C |

| | | |
|---|---|---|
| 261 | 261C > T | Genomic |
| 1871 | 1871C > T | Genomic |
| 2309 | 2309G > A | Genomic |
| 2384 | 2384C > T | Genomic |
| 2710 | 2710A > G | Genomic |
| 2831 | 2831G > A | Genomic |
| 3290 | 3290C > G | Genomic |
| 3541 | 3541G > A | Genomic |
| 4047 | 4047C > A | Genomic |

| | | | | |
|---|---|---|---|---|
| U10554 | U10554 | 118490 | GEN-LUP | Exon R of choline |

acetyltransferase and complete cds for vesicular acetylcholine transporter

| | | |
|---|---|---|
| 574 | 574T > C | Genomic |
| 607 | 607C > T | Genomic |
| 1679 | 1679T > A | Genomic |
| 1980 | 1980C > T | Genomic |
| 2081 | 2081A > G | Genomic |
| 2265 | 2265A > G | Genomic |
| 2338 | 2338A > C | Genomic |
| 2372 | 2372A > G | Genomic |
| 2440 | 2440T > C | Genomic |
| 2894 | 2894C > T | Genomic |
| 3512 | 3512C > T | Genomic |
| 3650 | 3650C > T | Genomic |
| 3666 | 3666G > C | Genomic |
| 3779 | 3779G > C | Genomic |
| 4270 | 4270C > G | Genomic |
| 4320 | 4320C > T | Genomic |
| 4848 | 4848C > A | Genomic |
| 4960 | 4960G > A | Genomic |
| 5041 | 5041G > A | Genomic |
| 5163 | 5163C > T | Genomic |
| 5331 | 5331T > C | Genomic |
| 5392 | 5392G > C | Genomic |
| 5440 | 5440C > T | Genomic |
| 5443 | 5443C > A | Genomic |
| 5751 | 5751T > C | Genomic |
| 5970 | 5970G > C | Genomic |

| | | | | |
|---|---|---|---|---|
| X56585 | X56585 | 118490 | GEN-LUU | Human gene for choline |

acetyltransferase (EC 2.3.1.6), partial

| | | | | | | | | | |
|---|---|---|---|---|---|---|---|---|---|
| 1728G | 1860C | 2266T | 2295C | 2422T | 2753G | 2881C | 3697A | 5435T | 5606A |
| 1728G | 1860C | 2266T | 2295C | 2422C | 2753G | 2881C | 3697A | 5435T | 5606A |
| 1728G | 1860C | 2266C | 2295C | 2422T | 3961C | 4571C | 4798A | 4804G | 4822C |
| 1728G | 1860C | 2266C | 2295C | 2422C | 2753G | 2881C | 3697A | 5435T | 5606A |
| 1728G | 1860C | 2266T | 2295T | 2422C | 2753G | 2881C | 3697G | 5435T | 5606A |
| 1728G | 1860C | 2266T | 2295T | 2422C | 2753G | 2881C | 3697A | 5435T | 5606A |
| 1728G | 1860C | 2266C | 2295C | 2422T | 4571C | 4798A | 4804C | 4822C | |
| 1728A | 1860C | 2266T | 2295C | 3961C | 4571C | 4798A | 4804G | 4822C | |
| 1728G | 1860T | 2295C | 2422T | 2753G | 5435T | 5606A | | | |
| 1728G | 1860T | 2266C | 2295T | 2422T | 2753G | 2881G | 3697G | 5435T | 5606A |
| 1728G | 1860C | 2266T | 2295C | 2422T | 2753G | 2881C | 3697A | 5435T | 5606A |
| 1728G | 1860C | 2266T | 2295T | 2422T | 2753G | 2881C | 3697G | 5435T | 5606A |
| 1728A | 1860C | 2266T | 2295C | 2422T | 4571C | 4798A | 4804C | 4822C | |
| 1728G | 1860C | 2266C | 2295C | 2422T | 3961C | 4571C | 4798A | 4804G | 4822C |
| 1728G | 1860C | 2266C | 2295C | 2422T | 2753A | 2881G | 3697A | 5435T | 5606A |
| 1728G | 1860C | 2266C | 2295T | 2422T | 2753G | 2881G | 3697G | 5435T | 5606A |
| 1728A | 1860C | 2266T | 2295C | 2422T | | | | | |
| 1728G | 1860C | 2266T | 2295C | 2422T | 3961G | 4571C | 4798A | 4804G | 4822C |
| 1728A | 1860C | 2266T | 2295C | 2422T | 2753G | 2881C | 5435T | 5606A | |
| 1728A | 1860C | 2266T | 2295T | 2422T | 2753G | 2881C | 3697G | 5435T | 5606A |
| 1728G | 1860C | 2266C | 2295C | 2422C | 2753G | 2881C | 3697A | 5435T | 5606A |
| 1728A | 1860C | 2266C | 2295C | 2422T | 3961C | 4571C | 4798A | 4804G | 4822C |
| 1728G | 1860C | 22660 | 2295C | 2422T | 3961C | 4571C | 4798A | 4804C | 4822C |
| 1728A | 1860C | 2266T | 2295C | 2422T | 3961C | 4571C | 4798A | 4804G | 4822C |
| 1728A | 2266T | 2295C | 2422T | 2753G | 2881C | 3697A | 5435T | 5606A | |
| 1728G | 1860C | 2266T | 2295C | 2422C | 2753G | 2881C | 3697A | 5435T | 5606A |
| 1728G | 1860C | 2266C | 2295T | 2422T | 3961G | 4571T | 4798A | 4804G | 4822C |
| 1728G | 1860C | 2266T | 2295T | 2422C | 2753G | 2881C | 3697G | 5435T | 5606A |
| 2266G | 2295C | 2422T | 3961C | 4571C | 4798A | 4804G | 4822C | | |
| 1728A | 1860C | 2266C | 2295C | 2422T | 2753G | 2881C | 3697A | 5435T | 5606A |
| 1728G | 1860T | 2266C | 2295C | 2422T | | | | | |
| 1728A | 1860C | 2266T | 2295C | 2422T | 2753G | 2881C | 3697A | 5435T | 5606A |
| 1728G | 1860C | 2266T | 2295T | 2422T | 3961G | 4571C | 4798A | 4804G | 4822T |
| 1728G | 1860C | 2266C | 2295C | 2422T | 3961G | 4571C | 4798A | 4804G | 4822C |
| 1728G | 1860C | 2266C | 2295C | 2422T | 2753G | 2881G | 3697A | 5435T | 5606A |
| 1728G | 1860C | 2266T | 2295T | 2422T | 3961C | 4571C | 4798A | 4804G | 4822C |
| 1728G | 1860T | 2266C | 2295C | 2422T | 2753G | 2881G | 5435T | 5606A | |
| 1728G | 1860C | 2266C | 2295C | 2422T | 2753G | 2881C | 3697A | 5435T | 5606G |
| 1728G | 1860C | 2266T | 2295T | 2422C | 3961G | 4571C | 4798A | 4804G | 4822T |
| 2728A | 1860C | 2266T | 2295C | 2422T | 2753G | 2881C | 3697A | 5435G | 5606A |
| 1728A | 1860C | 2266T | 2295T | 2422T | 3961G | 4571C | 4798A | 4804G | 4822C |
| 1728G | 1860C | 2266C | 2295C | 2422C | 3961C | 4571C | 4798A | 4804G | 4822C |
| 1728G | 1860C | 2266C | 2295C | 2422T | 2753G | 2881C | 3697A | 5435T | 5606A |

| | | |
|---|---|---|
| 1728 | 1728G > A | Genomic |
| 1860 | 1860C > T | Genomic |
| 2266 | 2266C > T | Genomic |
| 2295 | 2295C > T | Genomic |
| 2422 | 2422T > C | Genomic |
| 2753 | 2753G > A | Genomic |
| 2881 | 2881G > C | Genomic |
| 3697 | 3697A > G | Genomic |
| 3961 | 3961C > G | Genomic |
| 4571 | 4571C > T | Genomic |
| 4798 | 4798G > A | Genomic |
| 4804 | 4804G > C | Genomic |
| 4822 | 4822C > T | Genomic |
| 5435 | 5435T > G | Genomic |
| 5606 | 5606A > G | Genomic |

| | | | | |
|---|---|---|---|---|
| M96015 | M96015 | 118490 | GEN-LUZ | Human choline acetyltransferase |

gene, alternate exon 1 including 5′ end of cds

| | | | | | | |
|---|---|---|---|---|---|---|
| 445G | 564G | 1206T | 1258G | 1537G | 1839T | 2021C |
| 445G | 564G | 1206G | 1258G | 1537G | 1839T | 2021C |
| 1206T | 1258A | | | | | |
| 445G | 564G | 1206G | 1258A | 1537G | 1839G | 2021C |
| 564G | 1258G | 1537G | 1839T | 2021G | | |
| 445G | 1206G | 1537A | 1839T | 2021C | | |
| 445G | 564G | 1206G | 1258A | 1839T | 2021G | |
| 445G | 564G | 1206G | 1258A | 1537G | 1839T | 2021C |
| 445T | 564G | 1206T | 1258G | 1537G | 2021C | |
| 1206T | 1258G | | | | | |
| 1206T | 1258A | | | | | |
| 445T | 564G | 1206G | 1258G | 1537G | 1839T | 2021G |
| 445G | 564G | 1206G | 1258A | 1537A | 1839T | 2021C |
| 445G | 564A | 1206G | 1258G | 1537A | 1839T | 2021C |
| 445T | 564G | 1206G | 1258G | 1537G | 2021G | |
| 445G | 564G | 1206G | 1258G | 1537G | 1839G | 2021C |
| 445G | 564G | 1206T | 1258G | 1537G | 1839G | 2021C |
| 1206G | 1258A | | | | | |
| 445G | 564G | 1206G | 1258G | 1537G | 1839G | 2021G |
| 445T | 564G | 1206G | 1258G | 1537G | 1839T | 2021C |

| | | | |
|---|---|---|---|
| 445 | 445G > T | | Genomic |
| 564 | 564G > A | | Genomic |
| 1206 | 1206T > G | | Genomic |
| 1258 | 1258G > A | | Genomic |
| 1537 | 1537G > A | | Genomic |
| 1839 | 1839T > G | | Genomic |
| 2021 | 2021C > G G | enomic | |

| | | | | |
|---|---|---|---|---|
| L10819 | L10819 | 171150 | GEN-LVD | Homo sapiens aryl |

sulfotransferase mRNA, complete cds

| | | |
|---|---|---|
| 191 | 153C > T | Silent |
| 200 | 162G > A | Silent |
| 230 | 192T > C | Silent |
| 242 | 204G > A | Silent |
| 267 | 229A > G | M77V |
| 295 | 257C > T | A86V |
| 330 | 292G > A | D98N |
| 338 | 300G > A | Silent |
| 638 | 600C > G | Silent |
| 676 | 638A > G | H213R |
| 940 | 902G > A | 3′ |
| 1011 | 973T > C | 3′ |

| | | | | |
|---|---|---|---|---|
| L19956 | L19956 | 600641 | GEN-LVE | Human aryl sulfotransferase |

mRNA, complete cds

| | | |
|---|---|---|
| 243 | 105A > G | Silent |
| 284 | 146C > T | 549F |

| | | | | |
|---|---|---|---|---|
| X78282 | X78282 | 601292 | GEN-LVF | H. sapiens mRNA for aryl |

sulfotransferase (ST1A2)

| | | |
|---|---|---|
| 895 | 895T > C | 3′ |

| | | | | |
|---|---|---|---|---|
| L11695 | L11695 | 190181 | GEN-MDJ | Human activin receptor-like |

kinase (ALK-5) mRNA, complete cds

| | | |
|---|---|---|
| 1657 | 1581G > A | 3′ |

| | | | | |
|---|---|---|---|---|
| U03858 | U03858 | 600007 | GEN-MDM | Fms-related tyrosine kinase 3 |
| ligand | | | | |

| | | |
|---|---|---|
| 683 | 600C > T | Silent |
| 1016 | 933T > C | 3′ |

| | | | | |
|---|---|---|---|---|
| M37815 | M37815 | 186760 | GEN-MDZ | Human T-cell membrane |

glycoprotein CD28 mRNA

| | |
|---|---|
| 327G | 1061A |
| 327A | 1061A |
| 327G | 1061C |

| | | |
|---|---|---|
| 327 | 105G > A | Silent |
| 1061 | 839A > C | 3′ |

| X57303 | | X57303 | | 104615 | | GEN-MEB | | H. sapiens REC1L mRNA | | | | |
|---|---|---|---|---|---|---|---|---|---|---|---|---|
| | 474C | 573C | 582C | 630G | 1026G | 1059G | 1185C | 1332C | 1401C | 1551G | 1656T | 1672A |
| | 1747G | | | | | | | | | | | |
| | 474G | 582C | 630G | 1026G | 1059G | 1185C | 1332C | 1401C | 1551G | 1656T | 1672A | 1747A |
| | 474G | 573C | 582C | 630G | 1026G | 1059G | 1185C | 1332C | 1401G | 1551C | 1656C | 1672A |
| | 1747G | | | | | | | | | | | |
| | 474G | 573C | 582C | 630G | 1026G | 1059G | 1185C | 1332T | 1401C | 1551G | 1656T | 1672A |
| | 1747G | | | | | | | | | | | |
| | 573C | 582C | 630G | 1026G | 1059G | 1185T | 1332C | 1401C | 1551G | 1656T | 1672A | 1747G |
| | 474G | 573C | 582C | 630G | 1026G | 1059G | 1185C | 1332C | 1401G | 1551G | 1656C | 1672A |
| | 1747G | | | | | | | | | | | |
| | 573G | 1059G | 1185C | 1332C | 1401G | 1551G | 1747G | | | | | |
| | 474G | 573C | 582C | 630G | 1026G | 1059G | 1185C | 1332C | 1401C | 1656C | 1672A | 1747G |
| | 573C | 1059A | 1185C | 1332C | 1401G | 1551G | 1747G | | | | | |
| | 474G | 573C | 582C | 630G | 1026G | 1059A | 1185C | 1332C | 1401C | 1551G | 1656T | 1672A |
| | 1747G | | | | | | | | | | | |
| | 474G | 573C | 582C | 630G | 1026G | 1059G | 1185C | 1332C | 1401C | 1551G | 1656T | 1672A |
| | 1747G | | | | | | | | | | | |
| | 573C | 582C | 630G | 1026G | 1059A | 1185C | 1332T | 1401C | 1551G | 1656T | 1672A | 1747G |
| | 474G | 573G | 582C | 630G | 1026G | 1059G | 1185C | 1332C | 1401C | 1551G | 1656T | 1672A |
| | 1747G | | | | | | | | | | | |
| | 573C | 582C | 630G | 1026A | 1059G | 1185C | 1332C | 1401C | 1551C | 1656T | 1672A | 1747G |
| | 474G | 573G | 582C | 630G | 1026G | 1059G | 1185C | 1332C | 1401C | 1551C | 1656C | 1672A |
| | 1747G | | | | | | | | | | | |
| | 474C | 573C | 582C | 630G | 1026G | 1059A | 1185C | 1332C | 1401C | 1551G | 1656T | 1672A |
| | 1747G | | | | | | | | | | | |
| | 573G | 582C | 630G | 1026G | 1059G | 1185C | 1332C | 1401C | 1551C | 1656C | 1672A | 1747G |
| | 474C | 573C | 582C | 630G | 1026G | 1059G | 1185T | 1332C | 1401C | 1551G | 1656T | 1672A |
| | 1747G | | | | | | | | | | | |
| | 474G | 573G | 582C | 630G | 1026G | 1059G | 1185C | 1332C | 1401C | 1551G | 1656C | 1672A |
| | 1747G | | | | | | | | | | | |
| | 573G | 582C | 630G | 1059G | 1185C | 1332C | 1401C | 1551C | 1656C | 1672A | 1747G | |
| | 474G | 573C | 582C | 630G | 1026G | 1059G | 1185C | 1332C | 1401C | 1551C | 1656C | 1672A |
| | 1747G | | | | | | | | | | | |
| | 573G | 582C | 630G | 1026G | 1059A | 1185C | 1332C | 1401C | 1551G | 1656T | 1672A | 1747G |
| | 573C | 582C | 630G | 1026G | 1059A | 1185C | 1332T | 1401C | 1551G | 1656T | 1672A | 1747G |
| | 474G | 573C | 582C | 630G | 1026G | 1059G | 1185C | 1332C | 1401C | 1551G | 1656T | 1747G |
| | 573G | 582T | 630A | 1059A | 1185C | 1332C | 1401G | 1551G | 1656C | 1672G | 1747G | |
| | 573C | 582C | 630G | 1026G | 1059A | 1185C | 1332C | 1401C | 1551G | 1656T | 1672A | 1747G |
| | 573G | 582C | 630G | 1059G | 1185C | 1332C | 1401G | 1551G | 1656T | 1672A | 1747G | |
| | 474G | 573G | 582C | 630G | 1026G | 1059G | 1185C | 1332C | 1401C | 1551G | 1656T | 1672A |
| | 1747A | | | | | | | | | | | |
| | 573C | 582C | 630G | 1026A | 1059G | 1185C | 1332C | 1401C | 1551G | 1656T | 1672A | 1747G |

| | | |
|---|---|---|
| 474 | 324C > G | Silent |
| 573 | 423C > G | Silent |
| 582 | 432C > T | Silent |
| 630 | 480C > A | Silent |
| 1026 | 876G > A | Silent |
| 1059 | 909G > A | Silent |
| 1185 | 1035T > C | Silent |
| 1332 | 1182C > T | Silent |
| 1401 | 1251C > G | Silent |
| 1551 | 1401C > C | Silent |
| 1656 | 1506T > C | Silent |
| 1672 | 1522A > G | I508V |
| 1747 | 1597G > A | A533T |

| | | | | |
|---|---|---|---|---|
| M68895 | M68895 | 103735 | GEN-MH7 | Human alcohol dehydrogenase 6 |

gene, complete cds

| | | |
|---|---|---|
| 547 | 454G > A | V152M |

| | | | | |
|---|---|---|---|---|
| AF117815 | AF117815 | 603708 | GEN-MII | Homo sapiens molybdopterin |

synthase small and large subunit (MOCO1) bicistronic mRNA, complete cds

| | | |
|---|---|---|
| 1159 | 1159T > C | 3′ |

| | | | | |
|---|---|---|---|---|
| U23143 | U23143 | 138450 | GEN-MIY | Human mitochondrial serine |

hydroxymethyltransferase gene, nuclear encoded mitochondrion protein, complete
cds

| | | |
|---|---|---|
| 506 | 506T > G | F169C |

| | | | | |
|---|---|---|---|---|
| U68162 | U68162 | 159530 | GEN-MJM | Human thrombopoietin receptor |

(MPL) gene

| | | |
|---|---|---|
| 830G | 2749C | 3008G |
| 830A | 3008G | |
| 830G | 2749A | 3008G |
| 830G | 2749C | 3008A |
| 830G | 2749C | |
| 830A | 2749A | 3008G |
| 830G | 2749A | |

| | | |
|---|---|---|
| 830 | 764G > A | G255E |
| 2749 | 2683C > A | 3′ |
| 3008 | 2942G > A | 3′ |

| | | | | |
|---|---|---|---|---|
| X98332 | X98332 | 602607 | GEN-MMA | H. sapiens mRNA for organic |

cation transporter, liver

| | |
|---|---|
| 228T | 1294A |
| 228C | 1294G |
| 228C | 1294A |
| 228T | 1294G |

| | | |
|---|---|---|
| 228 | 156C > T | Silent |
| 630 | 558C > T | Silent |
| 1294 | 1222A > G | M408V |

| | | | | |
|---|---|---|---|---|
| AF058056 | AF058056 | 603654 | GEN-MNJ | Homo sapiens monocarboxylate |

transporter 2 (hMCT2) mRNA, complete cds

| | |
|---|---|
| 1460T | 1510C |
| 1460A | 1510C |
| 1460A | 1510T |

| | | |
|---|---|---|
| 200 | 73G > A | A25T |
| 203 | 76G > A | A26T |
| 588 | 461G > A | S154N |
| 1460 | 1333T > A | S445T |
| 1510 | 1383C > T | Silent |

| | | | | |
|---|---|---|---|---|
| U30930 | U30930 | 601291 | GEN-MOS | UDP glycosyltransferase 8 (UDP- |

galactose ceramide galactosyltransferase)

| | | | |
|---|---|---|---|
| 1256A | 1619G | 1758A | 2150T |
| 1256A | 1619G | 1758G | 2150C |
| 1256A | 1619A | 1758A | 2150C |
| 1256G | 1619G | 1758A | 2150C |
| 1256A | 1619G | 1758A | 2150C |
| 1256A | 1619G | 2150C | |

| | | |
|---|---|---|
| 1256 | 741A > G | Silent |
| 1619 | 1104G > A | M368I |
| 1758 | 1243A > G | K415E |
| 2150 | 1635C > T | 3′ |

| | | | | |
|---|---|---|---|---|
| U06088 | U06088 | 253000 | GEN-MP3 | Human N-acetylgalactosamine 6- |

sulphatase (GALNS) gene

| | | |
|---|---|---|
| 1936 | 1936C > T | 3′ |
| 2180 | 2180G > A | 3′ |
| 2221 | 2221G > A | 3′ |

| | | | | |
|---|---|---|---|---|
| AF039400 | AP039400 | 603906 | GEN-MQY | Homo sapiens calcium-dependent |

chloride channel-1 (hCLCA1) mRNA, complete cds

| | | |
|---|---|---|
| 996 | 645T > A | Silent |
| 2787 | 2436T > C | Silent |

| K01612 | | K01612 | | None | | GEN-MT4 | | Dihydrofolate reductase, | | | | |
|---|---|---|---|---|---|---|---|---|---|---|---|---|
| promoter | | | | | | | | | | | | |
| | 527A | 1120C | 1124G | 1678G | | | | | | | | |
| | 164A | 169A | 278T | 287G | 372A | 380A | 527G | 879C | 1120T | 1124G | 1135G | 1229G |
| | 1678C | | | | | | | | | | | |
| | 164C | 169A | 278T | 287G | 372A | 380A | 527A | 879C | 1120C | 1124G | 1135G | 1229G |
| | 1678C | | | | | | | | | | | |
| | 164C | 287G | 372A | 1120C | 1124G | 1229A | 1678C | | | | | |
| | 164C | 169A | 278T | 287G | 372A | 380A | 527G | 879C | 1120C | 1124G | 1135G | 1229G |
| | 1678C | | | | | | | | | | | |
| | 169A | 278T | 287A | 372A | 380A | 527G | 1120C | 1124G | 1135G | 1229G | | |
| | 169A | 278T | 287G | 372A | 380A | 527G | 918G | 925A | 1124G | 1135G | 1229G | 1678G |
| | 264C | 169A | 278T | 287G | 372A | 380A | 1120C | 1124A | 1135G | 1229G | 1678C | |
| | 169A | 278T | 287G | 372G | 380A | 918C | 925A | 1124G | 1135G | 1229G | 1678C | |
| | 164C | 169A | 278T | 287G | 372A | 380A | 527G | 918C | 925A | 1120C | 1124G | 1135A |
| | 1229A | 1678C | | | | | | | | | | |
| | 169G | 278C | 380T | 527A | 918G | 925G | 1678C | | | | | |
| | 264A | 169A | 278T | 287G | 372A | 380A | 527G | 879C | 1120C | 1124G | 1135G | 1229G |
| | 1678C | | | | | | | | | | | |
| | 264C | 169A | 278T | 287G | 372G | 380A | 527A | 918C | 925A | 1120C | 1124G | 1135G |
| | 1229G | 1678C | | | | | | | | | | |
| | 169A | 278T | 380A | 527A | 918G | 925A | 1120C | 1124G | 1135G | 1229A | 1678C | |
| | 164C | 169A | 278T | 287G | 372A | 380A | 527A | 1120C | 1124A | 1135G | 1229G | 1678C |
| | 164A | 169A | 278T | 287A | 372A | 380A | 527G | 112CC | 1124G | 1135G | 1229G | 1678G |
| | 169A | 278T | 380A | 527A | 918C | 925A | 1120C | 1124G | 1135A | 1229A | 1678C | |
| | 169A | 278T | 380A | 527A | 918C | 925A | 1120C | 1124G | 1135A | 1229G | 1678G | |
| | 164C | 169A | 278T | 287G | 372A | 380A | 527G | 918G | 925A | 1120C | 1124G | 1135G |
| | 1229G | 1678G | | | | | | | | | | |
| | 169A | 278T | 380A | 527G | 918C | 925A | 1120C | 1124G | 1135A | 1229G | 1678C | |
| | 164C | 169A | 278T | 287G | 372A | 380A | 527A | 879C | 1120C | 1124G | 1135G | 1229G |
| | 1678C | | | | | | | | | | | |
| | 164C | 169A | 278T | 287G | 372A | 380A | 527G | 879C | 1120C | 1124G | 1135G | 1229G |
| | 1678C | | | | | | | | | | | |
| | 164C | 169A | 278T | 287G | 372A | 380A | 527A | 918G | 925A | 1120C | 1124G | 1135G |
| | 1229G | 1678C | | | | | | | | | | |
| | 918C | 925G | 1120C | 1124G | 1135A | 1229A | 1678C | | | | | |
| | 164C | 169A | 278T | 287G | 372A | 380A | 527A | 879T | 1120C | 1124A | 1135G | 1229G |
| | 1678C | | | | | | | | | | | |
| | 169A | 278T | 380A | 527A | 918C | 925G | 1120C | 1124G | 1135A | 1229G | 1678C | |

| | | |
|---|---|---|
| 164 | 164C > A | Genomic |
| 169 | 169G > A | Genomic |
| 278 | 278C > T | Genomic |
| 287 | 287G > A | Genomic |
| 372 | 372A > G | Genomic |
| 380 | 38CT > A | Genomic |
| 527 | 527A > G | Genomic |
| 879 | 879C > T | Genomic |
| 918 | 918G > C | Genomic |
| 925 | 925G > A | Genomic |
| 1120 | 1120C > T | Genomic |
| 1124 | 1124G > A | Genomic |
| 1135 | 1135A > G | Genomic |
| 1229 | 1229A > G | Genomic |
| 1678 | 1678C > G | Genomic |

| | | | | |
|---|---|---|---|---|
| AF005216 | AF005216 | 147796 | +L,23 GEN-MT5 | Homo sapiens receptor-associated |

tyrosine kinase (JAK2) mRNA,complete cds

| | | | | | | | | | |
|---|---|---|---|---|---|---|---|---|---|
| 144C | 298T | 491C | 874A | 983T | 1641C | 1668G | 2423T | 2620A | 2984A |
| 144C | 491T | 874G | 983C | 1641C | 1668G | 2423T | 2620A | | |
| 144C | 298T | 491C | 874G | 983T | 1641C | 1668A | 2423T | 262CA | 2984A |
| 144C | 298C | 491C | 874G | 983C | 1641C | 1668G | 2423T | 2620A | 2984G |
| 144C | 298T | 491C | 874G | 983T | 1641C | 1668G | 2423C | 2620A | |
| 144C | 491C | 874G | 983C | 1641G | 1668G | 2423T | 2620A | | |
| 144C | 298T | 491C | 874G | 1641C | 1668G | 2423T | 2620C | 2984A | |
| 144C | 298T | 491C | 874G | 983C | 1641C | 1668G | 2423T | 2620A | 2984A |
| 144C | 298T | 491C | 874G | 983C | 1641C | 1668G | 2423T | 2620A | 2984G |
| 144C | 298T | 491C | 874G | 983T | 1641C | 1668G | 2423T | 2620A | 2984A |
| 144T | 298C | 491C | 874G | 983C | 1641C | 1668G | 2423T | 2620A | 2984G |
| 144C | 298T | 491C | 874G | 983T | 1641C | 1668G | 2620A | 2984G | |
| 144C | 298T | 491C | 874G | 983T | 1641C | 1668G | 2423C | 2620A | |
| 144C | 298T | 491C | 874G | 983C | 1641C | 1668G | 2423T | 2620A | 2984A |
| 144C | 298T | 491C | 874G | 983T | 1641C | 1668G | 2423T | 2620C | 2984A |
| 144C | 298T | 491T | 874G | 983C | 1641C | 1668G | 2423T | 2620A | 2984A |
| 144C | 298T | 491C | 874G | 983C | 2423T | 2620A | 2984G | | |
| 144T | 298T | 491C | 874G | 983C | 1641C | 1668G | 2423T | 2620A | 2984G |
| 144C | 298T | 874G | 983T | 1641C | 1668G | 2423T | 2620A | 2984A | |
| 144C | 298T | 491C | 874G | 983T | 2423T | 2620A | 2984A | | |

| | | |
|---|---|---|
| 144 | 144C > T | 3′ |
| 298 | 298C > T | 3′ |
| 491 | 491C > T | 3′ |
| 874 | 874G > A | 3′ |
| 983 | 983C > T | 3′ |
| 1641 | 1641C > G | 3′ |
| 1668 | 1668G > A | 3′ |
| 2423 | 2423T > C | 3′ |
| 2620 | 2620A > C | 3′ |
| 2984 | 2984A > G | 3′ |

| | | | | |
|---|---|---|---|---|
| AJ005200 | AJ005200 | None | GEN-MT6 | Homo sapiens MRP2 gene, promoter |
| region | | | | |

| | |
|---|---|
| 211G | 1206T |
| 211A | 1206C |
| 211G | 1206C |

| | | |
|---|---|---|
| 211 | 211A > G | Genomic |
| 1206 | 1206C > T | Genomic |

| | | | | |
|---|---|---|---|---|
| Y08062 | Y08062 | None | GEN-MT8 | Organic anion transporter, |
| promoter | | | | |

| | | | | | | |
|---|---|---|---|---|---|---|
| 310T | 689G | 726G | 799G | 908T | 1449T | 1470G |
| 310C | 689G | 726G | 799G | 908T | 1449T | 1470A |
| 310C | 689G | 726G | 799H | 908T | 1449C | 1470G |
| 310C | 689A | 908T | 1449T | 1470G | | |
| 310C | 689G | 726A | 799A | 908T | 1449T | 1470G |
| 310C | 689G | 726G | 799G | 908T | 1449T | 1470G |
| 310C | 726A | 799A | 908A | 1449T | 1470G | |
| 310C | 689A | 726A | 799A | 908A | 1449T | 1470G |
| 310C | 689G | 726A | 799A | 908T | | |
| 310C | 689G | 726A | 799A | 908T | 1449T | 1470A |
| 310C | 689G | 726A | 799A | 908T | 1449C | 1470G |
| 310C | 689A | 726A | 799A | 908T | 1449T | 1470G |

| | | |
|---|---|---|
| 310 | 310C > T | Genomic |
| 689 | 689G > A | Genomic |
| 726 | 726G > A | Genomic |
| 799 | 799G > A | Genomic |
| 908 | 908T > A | Genomic |
| 1449 | 1449T > C | Genomic |
| 1470 | 1470G > A | Genomic |
| 1702 | 1702\hat{ }insA | Genomic |

| | | | | |
|---|---|---|---|---|
| U08374 | U08374 | 600522 | GEN-MT9 | Human cytosolic phospholipase A2 |

(cPLA2) gene, promoter region

| | | |
|---|---|---|
| 588 | 588T > C | Genomic |

| | | | | |
|---|---|---|---|---|
| AF120161 | AF120161 | None | GEN-MTB | Retinoic X receptor beta, |

promoter and genomic

| | | |
|---|---|---|
| 107 | 107C > G | Genomic |
| 218 | 218C > T | Genomic |
| 2230 | 2230T > A | Genomic |
| 2352 | 2352T > C | Genomic |
| 3148 | 3148A > C | Genomic |
| 3148 | 3148A > T | Genomic |
| 3459 | 3459T > C | Genomic |
| 3558 | 3558C > T | Genomic |
| 3713 | 3713C > G | Genomic |
| 5462 | 5462C > T | Genomic |
| 5667 | 5667C > T | Genomic |
| 5865 | 5865G > A | Genomic |
| 6041 | 6041T > C | Genomic |
| 6544 | 6544C > T | Genomic |
| 6604 | 6604C > A | Genomic |
| 7048 | 7048G > T | Genomic |
| 7266 | 7266T > A | Genomic |
| 7279 | 7279T > G | Genomic |
| 7412 | 7412A > T | Genomic |
| 7804 | 7804T > A | Genomic |
| 7833 | 7833T > C | Genomic |
| 7834 | 7834A > C | Genomic |

| | | | | |
|---|---|---|---|---|
| Z29336 | Z29336 | None | GEN-MTC | Superoxide dismutase 1 (Cu/Zn), |
| promoter | | | | |

| | |
|---|---|
| 161G | 402T |
| 161A | 402C |
| 161G | 402C |

| | | |
|---|---|---|
| 161 | 161G > A | Genomic |
| 402 | 402C > T | Genomic |

| | | | | |
|---|---|---|---|---|
| Z35286 | Z35286 | None | GEN-MTD | Multidrug resistance protein 3 |

(MDR3), promoter

| | | | | | | | | |
|---|---|---|---|---|---|---|---|---|
| 286C | 459T | 481C | 796T | 1966C | 1985G | 2002T | | |
| 286C | 510A | 796C | 1966T | 1985C | 2002T | | | |
| 286C | 459T | 510A | 1966T | 1985G | 2002T | | | |
| 286C | 481C | 510A | 1966T | 2002C | | | | |
| 286C | 481C | 510A | 796C | 1966C | 1985G | 2002T | | |
| 286C | 459C | 481C | 510A | 796C | 1966T | 1985G | 2002T | |
| 286C | 459T | 481C | 510A | 796C | 1885G | 1966C | 1985C | 2002T |
| 286C | 459T | 481C | 510A | 796T | 1885G | 1966C | 1985C | 2002T |
| 286T | 459T | 481C | 796T | 1966C | 1985G | 2002T | | |
| 286C | 459T | 481C | 510A | 796C | 1885C | 1966C | 1985C | 2002C |
| 286C | 459T | 481C | 510A | 796T | 1885C | 1966C | 1985C | 2002T |
| 286C | 459T | 481C | 510A | 1885C | 1966T | 1985C | 2002C | |
| 286C | 459T | 481C | 510A | 796T | 1885G | 1966C | 1985G | 2002C |
| 286C | 796C | 1885C | 1966C | 1985C | 2002C | | | |
| 286C | 459T | 481A | 510A | 796T | 1966T | 1985G | 2002T | |
| 286T | 459T | 481C | 510G | 796T | 1966C | 1985G | 2002T | |
| 286C | 459T | 481C | 510A | 796T | 1885G | 1966C | 1985G | 2002T |
| 286C | 459C | 481C | 510A | 796C | 1966T | 1985G | 2002T | |
| 286C | 459T | 481A | 510A | 796C | 1885G | 1966T | 1985C | 2002T |
| 286C | 459C | 481C | 510A | 796C | 1885G | 1966C | 1985G | 2002T |
| 286C | 459T | 481C | 510A | 796T | 1885G | 1966C | 1985C | 2002C |

| | | |
|---|---|---|
| 286 | 286C > T | Genomic |
| 459 | 459T > C | Genomic |
| 481 | 481C > A | Genomic |
| 510 | 510A > G | Genomic |
| 796 | 796C > T | Genomic |
| 1885 | 1885C > G | Genomic |
| 1966 | 1966C > T | Genomic |
| 1985 | 1985C > G | Genomic |
| 2002 | 2002T > C | Genomic |

| M38191 | | M38191 | | None | | GEN-MTE | | 5-lipoxygenase, promoter | | | |
|---|---|---|---|---|---|---|---|---|---|---|---|
| | 84G | 137G | 168G | 351G | 559G | 940G | 943G | 1000G | 1085G | 1285T | |
| | 84G | 137G | 168A | 351G | 559G | 940G | 943G | 1000G | 1085C | 1285T | 1310C |
| | 84G | 137G | 168G | 351G | 559T | 940G | 943G | 1000G | 1085C | 1285T | 1310C |
| | 84G | 137G | 168G | 351G | 559G | 940A | 943G | 1000G | 1085C | 1285T | 1310C |
| | 84G | 137G | 168G | 351G | 559G | 940G | 943G | 1000G | 1085C | 1285T | 1310C |
| | 84A | 137A | 168G | 351A | 559T | 940G | 943G | 1000A | 1085C | 1285C | 1310C |
| | 84G | 137G | 168G | 351G | 559G | 940G | 943A | 1000G | 1085C | 1285T | 1310C |
| | 84A | 137A | 168G | 351G | 559T | 940G | 943G | 1000A | 1085C | 1285C | 1310C |
| | 84G | 137G | 168G | 351G | 559T | 940A | 943G | 1000G | 1085C | 1285T | 1310C |
| | 84A | 137A | 168G | 351G | 559G | 940G | 943G | 1000A | 1085C | 1285C | 1310C |
| | 84G | 137G | 168G | 351G | 559T | 940A | 943G | 1000G | 1085G | 1285T | 1310C |
| | 84G | 137G | 168G | 351G | 559G | 940G | 943G | 1000G | 1085G | 1285T | 1310T |
| | 559T | 940G | 943G | 1000G | 1085C | 1285T | 1310C | | | | |

| | | |
|---|---|---|
| 84 | 84G > A | Genomic |
| 137 | 137G > A | Genomic |
| 168 | 168G > A | Genomic |
| 351 | 351G > A | Genomic |
| 472 | 472-477de1GTTAAA | Genomic |
| 559 | 559G > T | Genomic |
| 940 | 940G > A | Genomic |
| 943 | 943G > A | Genomic |
| 1000 | 1000G > A | Genomic |
| 1085 | 1085C > G | Genomic |
| 1285 | 1285T > C | Genomic |
| 1310 | 1310C > T | Genomic |

| AL049595 | | AL049595 | | None | | GEN-MTI | | Serotonin receptor 5HT-1B, |
|---|---|---|---|---|---|---|---|---|
| promoter | | | | | | | | |
| | 73G | 287C | 295G | 302T | 462C | 793T | 900T | 1001A |
| | 73G | 287C | 295G | 302T | 462C | 793T | 900G | |
| | 73G | 287C | 295G | 302C | 462A | 793T | 900G | 1001A |
| | 302C | 462A | 900G | 1001T | | | | |
| | 73A | 287C | 295G | 302T | 462C | 793T | 900T | 1001A |
| | 73G | 287T | 302T | 462C | 793T | 900T | 1001A | |
| | 302C | 462A | 900G | 1001T | | | | |
| | 73G | 287C | 295G | 302T | 462C | 793G | 900T | 1001A |
| | 73A | 287C | 295G | 302C | 462A | 793T | 900G | 1001A |
| | 73G | 287C | 295G | 302T | 462C | 793T | 900G | 1001T |
| | 73G | 287T | 295G | 302T | 462C | 900T | 1001A | |
| | 73G | 287T | 295T | 302T | 462C | 793T | 900T | 1001A |

| | | |
|---|---|---|
| 73 | 73A > G | Genomic |
| 287 | 287C > T | Genomic |
| 295 | 295G > T | Genomic |
| 302 | 302T > C | Genomic |
| 462 | 462C > A | Genomic |
| 793 | 793T > G | Genomic |
| 900 | 900T > G | Genomic |
| 1001 | 1001A > T | Genomic |

| | | | | |
|---|---|---|---|---|
| U50136 | U50136 | 246530 | GEN-MTJ | Leukotriene C4 synthase, |

promoter and genomic

| | | | | | | | | | | | |
|---|---|---|---|---|---|---|---|---|---|---|---|
| 375G | 1003A | 1279G | 1342C | 1544T | 2154G | 2169C | 2406G | 2742C | 2779G | 3252T | 3416G |
| 3486C | 3603C | 3689G | 4010A | | | | | | | | |
| 375G | 1003A | 1279G | 1342C | 1544G | 1902A | 2154G | 2169C | 2406G | 2742C | 2779G | 2940C |
| 3252T | 3416G | 3486C | 3603T | 3689G | 4010A | | | | | | |
| 375G | 1003A | 1279G | 1342C | 1544T | 1902A | 2169C | 2406G | 2742C | 2779G | 2940C | 3252G |
| 3416G | 3486C | 3603T | 3689G | 4010A | | | | | | | |
| 375G | 1279G | 1342C | 1902A | 2154G | 2169T | 2779G | 2940C | 3252T | 3416G | 3486C | 3689G |
| 1003A | 1279A | 1342C | 1544T | 1902A | 2154G | 2169C | 2406G | 2742C | 2779G | 2940C | 3252T |
| 3416G | 3486C | 3603T | 3689G | 4010A | | | | | | | |
| 375G | 1003A | 1279G | 1342C | 1544T | 1902A | 2154G | 2169C | 2406A | 2742C | 2779G | 2940C |
| 3252T | 3416G | 3486C | 3603T | 3689G | 4010A | | | | | | |
| 375G | 1003A | 1279G | 1342T | 1544T | 1902A | 2154G | 2169C | 2406G | 2742C | 2779G | 2940C |
| 3252T | 3416G | 3486C | 3603C | 3689A | 4010A | | | | | | |
| 375G | 1003A | 1279G | 1342C | 1544T | 1902A | 2154T | 2169C | 2406G | 2742C | 2779G | 2940C |
| 3252T | 3416G | 3486C | 3603T | 3689G | 4010A | | | | | | |
| 375A | 1003A | 1279G | 1342C | 1544T | 1902A | 2154G | 2169C | 2406G | 2742C | 2779G | 2940C |
| 3252T | 3416G | 3486C | 3603T | 3689G | 4010A | | | | | | |
| 375G | 1003C | 1279G | 1342T | 1544T | 1902A | 2154G | 2169C | 2406G | 2742C | 2779G | 2940C |
| 3252T | 3416G | 3486C | 3603C | 3689A | 4010A | | | | | | |
| 375G | 1003A | 1279G | 1342C | 1544T | 1902A | 2154G | 2169C | 2406G | 2742C | 2779G | 2940C |
| 3252T | 3416A | 3486C | 3603T | 3689G | 4010A | | | | | | |
| 375G | 1279G | 1342C | 1544T | 1902A | 2154G | 2169C | 2406G | 2779G | 2940C | 3252T | 3416G |
| 3486T | 3689G | | | | | | | | | | |
| 375G | 1003A | 1279G | 1342C | 1544T | 1902A | 2154G | 2169C | 2406G | 2742C | 2779G | 2940A |
| 3252T | 3416G | 3486C | 3603T | 3689G | 4010A | | | | | | |
| 375G | 1003A | 1279G | 1342C | 1544T | 1902A | 2154G | 2169C | 2406G | 2742C | 2779G | 2940C |
| 3252T | 3416G | 3486C | 3603T | 3689G | 4010A | | | | | | |
| 375G | 1003C | 1279G | 1342C | 1544T | 1902A | 2154G | 2169C | 2406G | 2742T | 2940G | 3252T |
| 3416G | 3486C | 3603C | 3689G | | | | | | | | |
| 375G | 1279G | 1342C | 1902A | 2154G | 2169T | 2406A | 2742T | 2779G | 2940C | 3252T | 3416G |
| 3486C | 3603C | 3689G | 4010G | | | | | | | | |
| 375G | 1003C | 1279G | 1342C | 1544T | 1902A | 2154T | 2169C | 2406G | 2742T | 2779G | 2940C |
| 3252T | 3416G | 3486C | | | | | | | | | |
| 375G | 1003C | 1279G | 1342T | 1544T | 1902G | 2154G | 2169C | 2406G | 2940C | 3252T | 3486C |
| 3603C | 3689A | 4010A | | | | | | | | | |
| 375G | 1003C | 1279G | 1342C | 1544T | 1902A | 2154G | 2169C | 2406G | 2742T | 2779G | 2940C |
| 3252T | 3416G | 3486C | 3603C | 3689G | 4010G | | | | | | |
| 375G | 1003C | 1279G | 1342C | 1544T | 1902A | 2154G | 2169C | 2406G | 2742T | 2779G | 2940C |
| 3252T | 3416G | 3486T | 3603C | 3689G | 4010G | | | | | | |
| 375G | 1003C | 1279G | 1342C | 1544T | 1902A | 2154G | 2169C | 2406G | 2742T | 2779A | 2940C |
| 3252T | 3416G | 3486C | 3603C | 3689G | 4010G | | | | | | |
| 375G | 1003A | 1279G | 1342C | 1544T | 1902A | 2154T | 2169C | 2406G | 2742C | 2779G | 2940C |
| 3252G | 3416G | 3486C | 3603T | 3689G | 4010A | | | | | | |
| 375G | 1003A | 1279G | 1342C | 1544T | 2154G | 2169C | 2406G | 2742C | 2779G | 3252T | 3416G |
| 3486C | 3603C | 3689G | 4010A | | | | | | | | |
| 375A | 1003C | 1279G | 1342C | 1544T | 1902A | 2154G | 2169C | 2406G | 2742T | 2779G | 2940C |
| 3252T | 3416G | 3486C | 3603C | 3689G | 4010G | | | | | | |
| 375G | 1003C | 1279G | 1342C | 1544T | 1902A | 2154G | 2169C | 2406G | 2742T | 2940C | 3252T |
| 3416G | 3486G | 3603C | 3689G | | | | | | | | |
| 375A | 1003C | 1279G | 1342C | 1544T | 1902A | 2154G | 2169C | 2406G | 2742T | 2779G | 2940C |
| 3252T | 3416G | 3486T | 3603C | 3689G | 4010G | | | | | | |
| 375G | 1279A | 1342C | 1544T | 1902A | 2154G | 2169C | 2406G | 2742C | 2779G | 3252T | 3416G |
| 3486C | 3603T | 3689G | 4010A | | | | | | | | |

| | | |
|---|---|---|
| 375 | 375G > A | Genomic |
| 1003 | 1003A > C | Genomic |
| 1279 | 1279G > A | Genomic |
| 1342 | 1342C > T | Genomic |
| 1544 | 1544T > G | Genomic |
| 1902 | 1902A > G | Genomic |
| 2154 | 2154G > T | Genomic |
| 2169 | 2169C > T | Genomic |
| 2406 | 2406G > A | Genomic |
| 2742 | 2742C > T | Genomic |
| 2779 | 2779G > A | Genomic |
| 2940 | 2940C > A | Genomic |
| 2252 | 3252T > G | Genomic |
| 3416 | 3416G > A | Genomic |
| 3486 | 3486C > T | Genomic |
| 3603 | 3603T > C | Genomic |
| 3689 | 3689G > A | Genomic |
| 4010 | 4010A > G | Genomic |

| | | | | |
|---|---|---|---|---|
| M60470 | M60470 | 603700 | GEN-MTL | 5-lipoxygenase activating |

protein (FLAP) gene, promoter and exon 1

| | | | | | | |
|---|---|---|---|---|---|---|
| 185G | 188T | 336T | 41GT | 524G | 840G | 862A |
| 185G | 188T | 336T | 415T | 524G | 840G | 862C |
| 185G | 188T | 336G | 840G | | | |
| 185A | 188T | 336T | 415T | 524G | 840G | |
| 185G | 188T | 336T | 415T | 524G | 840A | 862A |
| 185G | 188C | 336T | 415T | 524G | 862A | |
| 185G | 188C | 336T | 415T | 524G | 840A | 862A |
| 185G | 188T | 336G | 415A | 524A | 840G | 862C |
| 185A | 188T | 336T | 415T | 524G | 840G | 862C |
| 185G | 188T | 336T | 415T | 524G | 840A | 862C |

| | | |
|---|---|---|
| 185 | 185G > A | Genomic |
| 188 | 188T > C | Genomic |
| 336 | 336T > G | Genomic |
| 415 | 415T > A | Genomic |
| 524 | 524G > A | Genomic |
| 840 | 840G > A | Genomic |
| 862 | 862C > A | Genomic |

| | | | | |
|---|---|---|---|---|
| M63259 | M63259 | 603700 | GEN-MTM | Human 5-lipoxygenase activating |

protein (FLAP) gene, exon 2

| | | | |
|---|---|---|---|
| 65G | 113G | 353C | 446T |
| 65A | 113T | 353A | 446T |
| 65A | 113T | 353A | 446C |
| 65A | 113T | 353C | 446T |
| 65G | 113T | 353C | 446T |

| | | |
|---|---|---|
| 65 | 65G > A | Genomic |
| 113 | 113T > G | Genomic |
| 353 | 353G > A | Genomic |
| 353 | 353G > C | Genomic |
| 446 | 446T > C | Genomic |

| | | | | |
|---|---|---|---|---|
| M63262 | M63262 | 603700 | GEN-MTN | Human 5-lipoxygenase activating |

protein (FLAP) gene, exon 5

| | | |
|---|---|---|
| 533 | 533A > G | Genomic |

| | | | | |
|---|---|---|---|---|
| M63261 | M63261 | 603700 | GEN-MTO | Human 5-lipoxygenase activating |

protein (FLAP) gene, exon 4

| | | |
|---|---|---|
| 440 | 440C > T | Genomic |

| | | | | |
|---|---|---|---|---|
| M63260 | M63260 | 603700 | GEN-MTP | Human 5-lipoxygenase activating |

protein (FLAP) gene, exon 3

| | | |
|---|---|---|
| 419 | 419G > A | Genomic |

| U65080 | | U65080 | | None | | GEN-MTR | | Leukotriene A4 hydrolase, | | |
|---|---|---|---|---|---|---|---|---|---|---|
| promoter | | | | | | | | | | |
| | 213G | 454C | 455C | 526G | 643T | 671T | 808C | 1115A | 1654A | 1754G |
| | 213G | 454C | 455G | 526G | 643T | 671T | 1115A | 1654A | 1754G | |
| | 454C | 455C | 526C | 643T | 671T | 808G | 1115C | 1654A | 1754G | |
| | 213G | 454C | 455C | 643T | 671T | 808C | 1115A | 1654A | 1754A | |
| | 454C | 455G | 526C | 643T | 671T | 808G | 1115C | 1654A | 1754G | |
| | 213G | 455C | 526C | 643T | 671T | 808G | 1115C | 1654G | | |
| | 213G | 454C | 455C | 526G | 643T | 671T | 808C | 1115C | 1654A | 1754G |
| | 213C | 454C | 455C | 526C | 643T | 808C | 1115C | 1654A | 1754A | |
| | 213G | 454C | 455C | 526C | 643T | 671T | 808G | 1115A | 1654A | 1754G |
| | 213G | 454C | 455C | 643T | 671T | 1115C | 1654A | 1754A | | |
| | 213G | 454T | 455C | 526G | 643T | 671T | 1115A | | | |
| | 213C | 454C | 455C | 808C | 1115A | 1654A | 1754A | | | |
| | 213G | 454C | 455C | 526G | 643T | 671T | 808C | 1115A | 1654A | 1754G |
| | 213C | 454T | 455C | 643T | 671T | 1115C | | | | |
| | 213C | 454C | 455C | 526C | 643A | 671A | 808C | 1115C | 1654A | 1754A |
| | 213C | 454C | 455C | 526C | 643T | 671T | 808C | 1115C | 1654A | 1754G |
| | 213G | 454C | 455C | 526G | 643T | 671T | 808G | 1115C | 1654A | 1754G |
| | 213G | 454T | 455C | 526C | 643T | 671T | 808G | 1115C | 1654G | 1754A |
| | 213G | 454T | 455C | 526G | 643T | 671T | 808G | 1115A | 1654G | 1754A |
| | 213G | 454C | 455C | 526C | 643T | 671T | 808G | 1115C | 1654A | 1754G |
| | 213G | 454C | 455C | 526C | 643T | 671T | 808C | 1115A | 1654A | 1754A |
| | 213G | 454T | 455C | 526G | 643T | 671T | 808G | 1115C | 1654G | 1754A |
| | 213C | 454C | 455C | 526C | 643T | 671T | 808G | 1115C | 1654A | 1754G |
| | 213G | 454C | 455C | 526C | 643T | 671T | 808C | 1115C | 1654A | 1754A |
| | 213C | 454C | 455C | 526C | 643A | 671A | 808C | 1115A | 1654A | 1754A |
| | 213G | 454C | 455C | 526G | 643T | 671T | 808C | 1115A | 1654A | 1754A |
| | 213G | 454C | 455G | 526G | 643T | 671T | 808G | 1115A | 1654A | 1754G |
| | 213C | 454T | 455C | 526G | 643T | 671T | 808G | 1115C | 1654G | 1754A |
| | 213G | 454C | 455C | 526G | 643T | 671T | 808G | 1115C | 1654A | 1754A |
| | 213C | 454C | 455C | 526C | 643T | 671T | 808C | 1115C | 1654A | 1754A |
| | 213G | 454C | 455G | 526C | 643T | 671T | 808G | 1115C | 1654A | 1754G |

| | | |
|---|---|---|
| 213 | 213G > C | Genomic |
| 454 | 454C > T | Genomic |
| 455 | 455C > G | Genomic |
| 526 | 526C > G | Genomic |
| 643 | 643T > A | Genomic |
| 671 | 671T > A | Genomic |
| 808 | 808C > G | Genomic |
| 1115 | 1115C > A | Genomic |
| 1654 | 1654A > G | Genomic |
| 1754 | 1754A > G | Genomic |

| | | | | |
|---|---|---|---|---|
| S77127 | S77127 | None | GEN-MTU | Superoxide dismutase 2 |

(manganese), promoter and genomic

| | | | |
|---|---|---|---|
| 333G | 485C | 745G | 888A |
| 333G | 485C | 745C | 888A |
| 333G | 485C | 745G | 888G |
| 333G | 485A | 745G | |
| 333A | 485A | 745C | 888A |
| 333G | 485A | 745C | 888A |
| 333G | 485A | 745G | 888A |
| 333G | 485A | 745G | 888G |

| | | |
|---|---|---|
| 333 | 333G > A | Genomic |
| 485 | 485A > C | Genomic |
| 745 | 745C > G | Genomic |
| 888 | 888A > G | Genomic |

| | | | | |
|---|---|---|---|---|
| AF088893 | AF088893 | None | GEN-MTX | Homo sapiens retinoic acid |

receptor alpha (RARA) gene, exon 7

| | |
|---|---|
| 97A | 355C |
| 97G | 355T |

| | | |
|---|---|---|
| 97 | 97A > G | Genomic |
| 355 | 355C > T | Genomic |

| | | | | |
|---|---|---|---|---|
| AF088890 | AF088890 | None | GEN-MU0 | Homo sapiens retinoic acid |

receptor alpha (RARA) gene, exon 3

| | | |
|---|---|---|
| 502 | 502C > G | Genomic |

| | | | | |
|---|---|---|---|---|
| AF088895 | AF088895 | None | GEN-MU1 | Homo sapiens retinoic acid |

receptor alpha (RARA) gene, exon 9 and complete cds

| | | |
|---|---|---|
| 197 | 197T > C | Genomic |

| | | | | |
|---|---|---|---|---|
| AF088888 | AF088888 | None | GEN-MU4 | Retinoic acid receptor alpha, |

promoter and exon 1

| | | |
|---|---|---|
| 992C | 1020T | 1157C |
| 992C | 1020C | 1157C |
| 992C | 1020C | 1157G |
| 992G | 1020C | 1157C |

| | | |
|---|---|---|
| 992 | 992C > G | Genomic |
| 1020 | 1020C > T | Genomic |
| 1157 | 1157C > G | Genomic |

| | | | | |
|---|---|---|---|---|
| AF091582 | AF091582 | 603201 | GEN-MU6 | Homo sapiens bile salt export |

pump (BSEP) mRNA, complete cds

| | | | | | | | | |
|---|---|---|---|---|---|---|---|---|
| 933T | 1083A | 1457C | 2155A | 2260T | 3733T | 4328G | 4460A | 4512G |
| 933T | 1083G | 1457T | 2155A | 2260T | 3733T | 4328G | 4460A | 4512G |
| 933T | 1083G | 1457T | 2l55A | 2260T | 3733T | 4328A | 4460G | 4512A |
| 933T | 1083A | 1457C | 2155G | 2260T | 3733T | 4328G | 4460A | 4512G |
| 933T | 1457C | 2155A | 2260T | 4328A | 4460A | | | |
| 933T | 1083A | 1457T | 2155A | 2260T | 3733T | 4328A | 4460G | 4512A |
| 933T | 1083A | 1457C | 2155A | 2260T | 3733T | 4328G | 4512A | |
| 933T | 1083A | 1457T | 2155G | 2260T | 3733T | 4328A | 4460G | 4512A |
| 933C | 1083A | 1457C | 2155A | 2260T | 3733T | 4328G | 4512G | |
| 933T | 1083A | 1457C | 2155A | 226CC | 3733T | 4328A | 4460G | 4512A |
| 933T | 1083A | 1457T | 2155A | 2260T | 3733T | 4328G | 4512A | |
| 933T | 1083G | 1457T | 2155A | 2260T | 3733T | 4328G | 4460G | 4512A |
| 933T | 1083A | 1457C | 2155A | 2260T | 3733T | 4328A | 4460G | 4512A |
| 933T | 1083A | 1457T | 3733A | 4328G | | | | |
| 933T | 1083G | 1457C | 2155G | 2260T | 3733T | | | |
| 933T | 1083A | 1457T | 2155A | 2260T | 3733T | 4328G | 4460A | 4512G |
| 933C | 1083A | 1457C | 2155A | 2260T | 3733T | 4328G | 4512A | |
| 933T | 1457C | 2155A | 2260T | 4328A | 4460A | 4512A | | |
| 933T | 1083A | 1457T | 2155A | 2260T | 3733T | 4328G | 4460G | 4512A |
| 933T | 1083A | 1457C | 2155A | 2260T | 3733T | 4328G | 4460G | 45l2A |
| 1083A | 1457C | 2155A | 2260T | 3733T | 4328A | 4460G | 4512A | |
| 1083A | 1457C | 3733T | 4328G | 4460A | 4512G | | | |
| 1083A | 1457C | 2155A | 2260T | 3733T | 4328G | 4460A | 4512G | |
| 933C | 1083A | 1457C | 2155A | 2260T | 3733T | 4328G | 4460G | 4512A |
| 933T | 1083A | 1457T | 2155A | 2260T | 3733T | 4328G | 4460A | 4512A |
| 933T | 1083A | 1457C | 3733T | 4328G | 4460G | 4512A | | |
| 4328A | 4460G | 4512A | | | | | | |
| 933T | 1083A | 1457T | 3733A | 4328G | 4460G | 4512A | | |
| 1083A | 1457C | 2155A | 2260T | 3733T | 4328G | 4460G | 4512A | |
| 933T | 1083G | 1457C | 2155G | 2260T | 3733T | 4328G | 4460A | 4512G |
| 933T | 1083A | 1457C | 3733T | 4328G | 4460A | 4512G | | |

| | | |
|---|---|---|
| 933 | 807T > C | Silent |
| 1083 | 957A > G | Silent |
| 1457 | 1331T > C | V444A |
| 2155 | 2029A > G | M677V |
| 2260 | 2134T > C | Silent |
| 3733 | 3607T > A | S1203T |
| 4328 | 4202G > A | 3′ |
| 4460 | 4334A > G | 3′ |
| 4512 | 4386G > A | 3′ |

| | | | | |
|---|---|---|---|---|
| AC004590 | AC004590 | None | GEN-MU7 | Multidrug resistance-associated |

protein 3 (MRP3), promoter

| | | | | | | | | | | |
|---|---|---|---|---|---|---|---|---|---|---|
| 56A | 149A | 690A | 735G | 1325G | 1764G | 1879C | 1958T | 2527G | 2832T | 2881T |
| 149A | 690A | 1764G | 1879G | 1958C | 2527G | 2881C | | | | |
| 56A | 149A | 690C | 1764G | 1879C | 1958C | 2527G | | | | |
| 56A | 149A | 690A | 735A | 1325G | 1764G | 1879C | 1958C | 2527G | 2832T | 2881T |
| 56A | 149A | 690A | 735A | 1325G | 1764G | 1879C | 1958C | 2527G | 2832T | 2881C |
| 56A | 149A | 690A | 735A | 1325G | 1764G | 1879C | 1958C | 2527A | 2832T | 2881T |
| 56A | 149A | 690A | 735G | 1764G | 1879C | 1958C | 2527G | 2832A | | |
| 56A | 149G | 690A | 735G | 1325G | 1764G | 1879C | 1958C | 2527G | 2832T | 2881T |
| 56A | 149A | 690A | 735A | 1325G | 1764A | 1879C | 1958C | 2527G | 2832T | |
| 56A | 149A | 690A | 735G | 1325G | 1764G | 1879C | 1958C | 2527G | 2832T | 2881T |
| 56G | 149A | 690A | 1325G | 1764G | 1879C | 1958C | 2527G | 2832T | 2881T | |
| 56G | 149A | 690A | 735G | 1325A | 1764G | 1879G | 1958C | 2527G | 2832A | 2881C |
| 149A | 690A | 735G | 1325A | 1764G | 1879C | 1958C | 2527G | 2832A | 2881C | |
| 56A | 149A | 690A | 735G | 1325A | 1764G | 1879C | 1958C | 2527G | 2832A | 2881C |
| 56G | 149A | 690A | 735G | 1325G | 1764G | 1879C | 1958C | 2527G | 2832T | 2881T |
| 56A | 149A | 690A | 735A | 1325G | 1764A | 1879C | 1958C | 2527G | 2832T | 2881C |
| 56A | 149A | 690C | 735G | 1325A | 1764G | 1879C | 1958C | 2527G | 2832A | 2881C |
| 56A | 149A | 690A | 735G | 1325G | 1764G | 1879C | 1958C | 2527G | 2832T | 2881C |

| | | |
|---|---|---|
| 56 | 56A > G | Genomic |
| 149 | 149A > G | Genomic |
| 690 | 690A > C | Genomic |
| 735 | 735A > G | Genomic |
| 1325 | 1325G > A | Genomic |
| 1764 | 1764G > A | Genomic |
| 1879 | 1879C > G | Genomic |
| 1958 | 1958C > T | Genomic |
| 2527 | 2527G > A | Genomic |
| 2832 | 2832T > A | Genomic |
| 2881 | 2881C > T | Genomic |

| | | | | |
|---|---|---|---|---|
| AC007022 | AC007022 | None | GEN-MU8 | Serotonin receptor 5-HT2C, |
| promoter | | | | |

| | | | |
|---|---|---|---|
| 198G | 260A | 498T | 872C |
| 198C | 260G | 498C | 872A |
| 260A | 498C | | |
| 198G | 260G | 498C | 872C |
| 198G | 260G | 872C | |
| 198C | 260A | 498C | 872A |
| 198C | 260A | 498T | 872A |

| | | |
|---|---|---|
| 198 | 198C > G | Genomic |
| 260 | 260G > A | Genomic |
| 498 | 498C > T | Genomic |
| 872 | 872A > C | Genomic |

| | | | | |
|---|---|---|---|---|
| U01824 | U01824 | None | GEN-MU9 | Human glutamate/aspartate |

transporter II mRNA, complete cds

| | |
|---|---|
| 754A | 1372G |
| 754G | 1372G |
| 754G | 1372A |
| 754A | 1372A |

| | | |
|---|---|---|
| 754 | 576G > A | Silent |
| 1372 | 1194A > G | Silent |

| | | | | |
|---|---|---|---|---|
| S70609 | S70609 | 601019 | GEN-MUB | glycine transporter type 1b |

[human, substantia nigra, mENA, 2364 nt]

| | |
|---|---|
| 927G | 1556G |

| | | |
|---|---|---|
| 927 | 694G > A | A232T |
| 1217 | 984C > T | Silent |
| 1556 | 1323G > A | Silent |

| | | | | |
|---|---|---|---|---|
| X87816 | X87816 | None | GEN-MUC | Cystathionine-beta-synthase, |
| promoters | | | | |

| | | | | |
|---|---|---|---|---|
| 173G | 181T | 231A | 571G | 683A |
| 173A | 181T | 231C | 571G | |
| 173G | 181T | 231C | 571G | 683C |
| 173G | 181T | 231C | 571C | |
| 173G | 181T | 231C | 571G | 683A |
| 173G | 181C | 231C | 571G | 683A |
| 571G | 683C | | | |
| 173A | 181T | 231C | 571G | 683C |
| 173G | 181T | 231C | 571C | 683C |

| | | |
|---|---|---|
| 173 | 173G > A | Genomic |
| 181 | 181T > C | Genomic |
| 231 | 231C > A | Genomic |
| 571 | 571G > C | Genomic |
| 683 | 683A > C | Genomic |

| | | | | |
|---|---|---|---|---|
| L12178 | L12178 | 308380 | GEN-MUF | Human interleukin 2 receptor |

gamma chain (IL2RG) gene, exon 1 and promoter region

| | |
|---|---|
| 270G | 720A |
| 270C | 720A |
| 270G | 720T |

| | | |
|---|---|---|
| 270 | 270G > C | Genomic |
| 720 | 720A > T | Genomic |

| | | | | |
|---|---|---|---|---|
| L19546 | L19546 | 308380 | GEN-MUG | Interleukin-2 receptor gamma |

chain, genomic sequence (not including promoter)

| | | |
|---|---|---|
| 377G | 389C | 885G |
| 389G | 885G | |
| 377G | 389G | 885A |
| 377T | 389G | 885A |
| 377G | 389C | 885A |
| 377G | 389G | 885G |

| | | |
|---|---|---|
| 377 | 377T > G | Genomic |
| 389 | 389G > C | Genomic |
| 885 | 885A > G | Genomic |

| | | | | |
|---|---|---|---|---|
| AF185589 | AF185589 | 124010 | GEN-MVA | Homo sapiens cytochrome P450 3A4 |

(CYP3A4) gene, promoter region

| | | |
|---|---|---|
| 355 | 355A > G | Genomic |
| 763 | 763A > G | Genomic |
| 3113 | 3113T > C | Genomic |
| 3262 | 3262G > T | Genomic |
| 3782 | 3782A > G | Genomic |
| 3798 | 3798C > T | Genomic |
| 4245 | 4245A > G | Genomic |
| 4257 | 4257G > A | Genomic |
| 4394 | 4394G > A | Genomic |
| 4587 | 4587C > A | Genomic |
| 5414 | 5414G > C | Genomic |
| 5458 | 5458A > G | Genomic |
| 5546 | 5546A > G | Genomic |
| 5730 | 5730T > C | Genomic |
| 6303 | 6303T > G | Genomic |
| 6559 | 6559G > A | Genomic |
| 6577 | 6577G > C | Genomic |
| 7293 | 7293A > T | Genomic |
| 8653 | 8653C > T | Genomic |
| 9304 | 9304C > T | Genomic |
| 9315 | 9315C > T | Genomic |
| 9318 | 9318C > T | Genomic |
| 9335 | 9335T > C | Genomic |
| 9338 | 9338T > C | Genomic |
| 9340 | 9340G > T | Genomic |
| 9825 | 9825G > G | Genomic |
| 10180 | 10180A > G | Genomic |
| 10186 | 10186A > G | Genomic |

| | | | | |
|---|---|---|---|---|
| U04636 | U04636 | 600262 | GEN-MVG | Cyclooxygenase 2, genomic |

sequence (not including promoter)

| | | |
|---|---|---|
| 227 | 227T > C | Genomic |
| 322 | 322C > T | Genomic |
| 671 | 671C > G | Genomic |
| 774 | 774C > G | Genomic |
| 841 | 841T > G | Genomic |
| 848 | 848T > A | Genomic |
| 1080 | 1080C > G | Genomic |
| 1167 | 1167C > T | Genomic |
| 1290 | 1290G > T | Genomic |
| 1379 | 1379T > C | Genomic |
| 1639 | 1639G > T | Genomic |
| 1985 | 1985T > c | Genomic |
| 2016 | 2016C > A | Genomic |
| 2033 | 2033C > G | Genomic |
| 2191 | 2191C > G | Genomic |
| 2231 | 2231C > T | Genomic |
| 2315 | 2315G > C | Genomic |
| 3068 | 3068A > G | Genomic |
| 3121 | 3121T > A | Genomic |
| 3250 | 3250G > A | Genomic |
| 3810 | 3810C > T | Genomic |
| 3989 | 3989T > G | Genomic |
| 4065 | 4065T > G | Genomic |
| 4383 | 4383G > A | Genomic |
| 4461 | 4461G > A | Genomic |
| 4505 | 4505T > C | Genomic |
| 4719 | 4719T > C | Genomic |
| 4900 | 4900T > C | Genomic |
| 5106 | 5106G > A | Genomic |
| 5310 | 5310T > C | Genomic |
| 5593 | 5593G > T | Genomic |
| 5756 | 5756C > T | Genomic |
| 6106 | 6106G > A | Genomic |
| 6251 | 6251G > A | Genomic |
| 6429 | 6429T > A | Genomic |
| 6438 | 6438T > C | Genomic |
| 7150 | 7150G > C | Genomic |
| 7959 | 7959C > T | Genomic |

| | | | | |
|---|---|---|---|---|
| A2002455 | AB002455 | 601270 | GEN-MVH | Leukotriene B4 omega-hydroxylase |

(CYP4F3), promoter

| | | | | | | | | | | | |
|---|---|---|---|---|---|---|---|---|---|---|---|
| 424G | 685T | 709G | 1335A | 1423A | 1612C | 1840C | 2103T | 2433G | 2476C | 2506G | 2559A |
| 2673T | | | | | | | | | | | |
| 424A | 685T | 709G | 1335A | 1423A | 1612C | 1840C | 2103C | 2433T | 2476C | 2506G | 2559G |
| 2673C | | | | | | | | | | | |
| 424G | 685T | 709G | 1335A | 1423A | 1612C | 1840C | 2103C | 2433T | 2476C | 2506G | 2559G |
| 2673C | | | | | | | | | | | |
| 424G | 685T | 709G | 1335A | 1612C | 1840C | 2103T | 2433G | 2476C | 2506G | 2673C | |
| 685T | 709G | 1335A | 1612T | 1840C | 2103T | 2433C | 2476C | 2506G | | | |
| 424A | 685T | 709G | 1335G | 1423A | 1612C | 1840C | 2103C | 2433T | 2476C | 2506G | 2559G |
| 2673C | | | | | | | | | | | |
| 685T | 709G | 1612C | 1840T | 2103C | 2433T | 2476C | 2506G | 2559G | 2673C | | |
| 424A | 685T | 709G | 1335A | 1423A | 1612C | 1840C | 2103T | 2433G | 2476C | 2506G | 2673T |
| 424A | 685T | 709G | 1335A | 1612C | 1840C | 2103T | 2433T | 2476C | 2506G | 2559G | 2673C |
| 685T | 709G | 1335A | 1423G | 1612C | 1840C | 2103C | 2433T | 2476C | 2506G | 2559G | 2673C |
| 424G | 685T | 709A | 1335A | 1423A | 1612C | 1840C | 2103T | 2433G | 2476C | 2506G | |
| 424G | 685T | 709G | 1335A | 1423G | 1612C | 1840C | 2103T | 2433G | 2476C | 2506G | 2559A |
| 2673T | | | | | | | | | | | |
| 424A | 685T | 709G | 1612C | 1840C | 2103C | 2433T | 2476T | 2506G | 2559G | 2673C | |
| 424A | 685T | 709G | 1335A | 1423A | 1612C | 1840C | 2103T | 2433T | 2476C | 2506G | 2673T |
| 685T | 709G | 1335G | 1612C | 1840C | 2103T | 2476C | 2506G | | | | |
| 685T | 709G | 1335A | 1423G | 1612C | 1840C | 2103C | 2433T | 2476C | 2506G | 2673T | |
| 424G | 685T | 709G | 1335A | 1612C | 1840C | 2103T | 2433T | 2476C | 2506G | 2559G | 2673C |
| 424G | 685C | 709G | 1335A | 1423G | 1612C | 2476C | | | | | |
| 424G | 685T | 709A | 1335A | 1423A | 1612C | 1840C | 2103T | 2433G | 2476C | 2506G | 2559G |
| 2673C | | | | | | | | | | | |
| 424G | 685C | 709G | 1335A | 1423G | 1612C | 1840T | 2103C | 2433T | 2476C | 2506T | 2559G |
| 2673C | | | | | | | | | | | |
| 424A | 685T | 709G | 1335A | 1423A | 1612C | 1840C | 2103T | 2433G | 2476C | 2506G | 2559A |
| 2673T | | | | | | | | | | | |
| 424A | 685T | 709G | 1335A | 1423G | 1612C | 1840C | 2103T | 2433T | 2476C | 2506G | 2559G |
| 2673C | | | | | | | | | | | |
| 424A | 685T | 709G | 1335A | 1423G | 1612C | 1840C | 2103C | 2433T | 2476C | 2506G | 2559A |
| 2673T | | | | | | | | | | | |
| 424G | 685T | 709G | 1335A | 1423A | 1612C | 1840C | 2103T | 2433T | 2476C | 2506G | 2559G |
| 2673C | | | | | | | | | | | |
| 424A | 685T | 709G | 1335A | 1423A | 1612C | 1840C | 2103T | 2433T | 2476C | 2506G | 2559G |
| 2673T | | | | | | | | | | | |
| 424G | 685T | 709G | 1335G | 1423G | 1612C | 1840T | 2103C | 2433T | 2476C | 2506G | 2559G |
| 2673C | | | | | | | | | | | |
| 424A | 685T | 709G | 1335G | 1423G | 1612C | 1840C | 2103C | 2433T | 2476C | 2506G | 2559G |
| 2673C | | | | | | | | | | | |
| 424A | 685T | 709G | 1335A | 1423A | 1612C | 1840C | 2103T | 2433T | 2476C | 2506G | 2559G |
| 2673C | | | | | | | | | | | |
| 424G | 685T | 709G | 1335A | 1423G | 1612C | 1840C | 2103C | 2433T | 2476C | 2506G | 2559G |
| 2673C | | | | | | | | | | | |
| 424A | 685T | 709G | 1335G | 1423G | 1612C | 1840C | 2103C | 2433T | 2476T | 2506G | 2559G |
| 2673C | | | | | | | | | | | |
| 424A | 685T | 709G | 1335G | 1423A | 1612C | 1840C | 2103T | 2433T | 2476C | 2506G | 2559G |
| 2673C | | | | | | | | | | | |
| 424G | 685T | 709A | 1335G | 1423A | 1612C | 1840C | 2103T | 2433G | 2476C | 2506G | 2559A |
| 2673T | | | | | | | | | | | |
| 424G | 685T | 709G | 1335A | 1423A | 1612C | 1840C | 2103T | 2433G | 2476C | 2506G | 2559G |
| 2673C | | | | | | | | | | | |
| 424A | 685T | 709G | 1335A | 1423A | 1612T | 1840C | 2103T | 2433G | 2476C | 2506G | 2559G |
| 2673C | | | | | | | | | | | |
| 424G | 685T | 709G | 1335A | 1423G | 1612C | 1840C | 2103T | 2433G | 2476C | 2506G | 2559G |
| 2673C | | | | | | | | | | | |

| | | |
|---|---|---|
| 424 | 424A > G | Genomic |
| 685 | 685T > C | Genomic |
| 709 | 709G > A | Genomic |
| 1335 | 1335G > A | Genomic |
| 1423 | 1423A > G | Genomic |
| 1612 | 1612C > T | Genomic |
| 1840 | 1840C > T | Genomic |
| 2103 | 2103T > C | Genomic |
| 2433 | 2433G > T | Genomic |
| 2476 | 2476C > T | Genomic |
| 2506 | 2506G > T | Genomic |
| 2559 | 2559A > G | Genomic |
| 2673 | 2673T > C | Genomic |

| | | | | |
|---|---|---|---|---|
| AF209389 | AF209389 | 124010 | GEN-MVI | Homo sapiens cytochrome P450 |

IIIA4 (CYP3A4) gene, exons 1 through 13 and complete cds

| | | |
|---|---|---|
| 732 | 732T > C | Genomic |
| 755 | 755C > T | Genomic |
| 1870 | 1870A > G | Genomic |
| 1925 | 1925A > G | Genomic |
| 2253 | 2253G > C | Genomic |
| 2444 | 2444A > G | Genomic |
| 2523 | 2523A > G | Genomic |
| 3136 | 3136C > T | Genomic |
| 3352 | 3352G > A | Genomic |
| 4768 | 4768A > T | Genomic |
| 4808 | 4808G > T | Genomic |
| 7208 | 7208T > A | Genomic |
| 7445 | 7445A > G | Genomic |
| 11923 | 11923G > A | Genomic |
| 13115 | 13115T > G | Genomic |
| 15105 | 15105T > C | Genomic |
| 15746 | 15746C > T | Genomic |
| 15871 | 15871T > G | Genomic |
| 15955 | 15955T > A | Genomic |
| 16095 | 16095T > C | Genomic |
| 16149 | 16149G > A | Genomic |
| 16363 | 16363C > T | Genomic |
| 17890 | 17890C > T | Genomic |
| 17997 | 17997C > G | Genomic |
| 18651 | 18651T > G | Genomic |
| 19100 | 19100A > T | Genomic |
| 20178 | 20178T > C | Genomic |
| 20338 | 20338G > A | Genomic |
| 22651 | 22651G > A | Genomic |
| 23187 | 23187C > T | Genomic |
| 23489 | 23489G > C | Genomic |

| | | | | |
|---|---|---|---|---|
| AJ012376 | AJ012376 | 600046 | GEN-MVJ | Homo sapiens mRNA for ATP |

binding cassette transporter-1 (ABC-1)

| | | | | | | | | | | | |
|---|---|---|---|---|---|---|---|---|---|---|---|
| 876C | 888G | 2251G | 2260A | 2413G | 2589A | 2754G | 3099T | 3304C | 3456G | 3573A | 3624G |
| 4162C | 4221G | 4230G | 4700A | 6580G | 6666G | | | | | | |
| 876C | 888A | 1980C | 2260A | 2589A | 3099T | 3304C | 3456C | 3624G | 4162C | 4221G | 4230G |
| 4700A | 6580G | 6666G | | | | | | | | | |
| 876C | 888G | 2251G | 2260A | 2413G | 2589A | 2754G | 3099T | 3304C | 3456G | 3573A | 3624G |
| 4162C | 4221G | 4230G | 4700G | 6580A | 6666G | | | | | | |
| 876C | 1980C | 2251G | 2260A | 2413G | 2589A | 2754G | 3099T | 3456G | 3573A | 3624A | 4162C |
| 4221G | 4230G | 4700A | 6580G | 6666G | | | | | | | |
| 876C | 888G | 2251G | 2260A | 2413G | 2589G | 2754G | 3099T | 3304C | 3573A | 4162C | 4221G |
| 4230G | 4700A | 6666A | | | | | | | | | |
| 2251G | 2260A | 2413G | 2589G | 2754G | 3099T | 3304C | 3456C | 3624G | 4162C | 4221G | 4230G |
| 4700A | 6580G | 6666G | | | | | | | | | |
| 876C | 888G | 1980C | 2251G | 2260A | 2413G | 2754A | 3099T | 3304C | 3456G | 3573A | 4700A |
| 6580G | 6666G | | | | | | | | | | |
| 1980C | 2413A | 2589G | 2754G | 3573A | 3624G | 4162C | 4221G | 4230G | 4700A | 6580G | 6666G |
| 876C | 2251A | 2260A | 2413G | 2754G | 3099T | 3304C | 3456G | 3573A | 3624G | 4162C | 4221G |
| 4230G | 6580G | 6666G | | | | | | | | | |
| 876C | 888A | 2251G | 2260A | 2413G | 2589A | 2754G | 3099T | 3304C | 3456G | 3573A | 3624G |
| 4162C | 4221G | 4230G | 4700G | 6580G | 6666G | | | | | | |
| 876C | 888G | 2251G | 2260A | 2413G | 2589A | 2754G | 3099T | 3304C | 3456G | 3573A | 3624G |
| 4162T | 4221G | 4230G | 4700G | 6580A | 6666G | | | | | | |
| 876C | 1980C | 2251G | 2260A | 2413G | 2589A | 2754G | 3099G | 3304C | 3456G | 3573A | 3624G |
| 4162C | 4221G | 4230G | 6580G | 6666G | | | | | | | |
| 876C | 1980C | 2251G | 2260A | 2413G | 2589A | 2754G | 3099T | 3304C | 3573A | 3624G | 4162G |
| 4221G | 4230T | 6580G | 6666G | | | | | | | | |
| 876C | 888G | 2251G | 2260A | 2413G | 2589G | 2754G | 3099T | 3304C | 3456G | 3573A | 3624G |
| 4162C | 4221G | 4230G | 4700G | 6580G | 6666G | | | | | | |
| 876C | 888G | 1980C | 2251G | 2260A | 2413G | 2589A | 2754G | 3099T | 3304C | 3456G | 3573A |
| 3624G | 4162C | 4221G | 4230G | 4700G | 6580G | 6666G | | | | | |
| 876C | 888G | 1980A | 2251G | 2260A | 2413G | 2589G | 2754G | 3099T | 3304C | 3456G | 3573A |
| 3624G | 4162C | 4221A | 4230G | 4700G | 6580G | 6666G | | | | | |
| 876C | 888A | 1980C | 2260A | 2589A | 3099T | 3304C | 3456C | 3624G | 4162C | 4221G | 4230G |
| 4700G | 6580G | 6666G | | | | | | | | | |
| 2251G | 2260A | 2413G | 2589A | 2754G | 3099T | 3304C | 3456G | 3573A | 3624G | 4162C | 4221A |
| 4230G | 4700G | 6580G | 6666G | | | | | | | | |
| 876C | 888G | 2251G | 2260A | 2413G | 2589G | 2754G | 3099T | 3304C | 3456G | 3573A | 3624G |
| 4162C | 4230G | 4700A | 6666G | | | | | | | | |
| 876C | 888G | 1980A | 2413A | 2589G | 2754G | 3099T | 3304C | 3456G | 3573A | 3624G | 4162C |
| 4221G | 4230G | 4700G | 6666G | | | | | | | | |
| 876C | 2251G | 2260A | 2413G | 2754G | 3099T | 3304T | 3456G | 3573A | 3624G | 4162C | 4221G |
| 4230G | 4700A | 6580G | 6666G | | | | | | | | |
| 1980A | 2413A | 2589G | 2754G | 3573A | 3624G | 4162C | 4221G | 4230G | 4700A | 6580G | 6666G |
| 876C | 2260C | 2413G | 2589A | 2754G | 3099T | 3304G | 3456G | 3573A | 3624G | 4162C | 4221G |
| 4230G | 4700G | 6580G | 6666G | | | | | | | | |
| 876C | 888G | 1980C | 2251G | 2260A | 2413G | 2589G | 2754G | 3099T | 3304C | 3456G | 3573A |
| 3624G | 4162C | 4221G | 4230G | 4700G | 6580G | 6666G | | | | | |
| 876C | 888G | 2251G | 2260A | 2413G | 2589A | 2754G | 3099T | 3304C | 3456G | 3573A | 3624G |
| 4162C | 4221G | 4230G | 4700G | 6580A | 6666G | | | | | | |
| 876C | 888G | 2251G | 2260A | 2413G | 2589G | 2754G | 3099T | 3304C | 3456C | 3573A | 3624A |
| 4162C | 4221G | 4230G | 4700A | 6666A | | | | | | | |
| 876C | 888A | 2251A | 2260C | 2413G | 2589A | 2754G | 3099T | 3304C | 3456G | 3573A | 3624G |
| 4162C | 4221G | 4230G | 4700G | 6580G | 6666G | | | | | | |
| 876C | 888A | 1980C | 2251G | 2260A | 2413G | 2589A | 2754G | 3099G | 3304C | 3456G | 3573A |
| 3624G | 4162C | 4221G | 4230G | 6580G | 6666G | | | | | | |
| 876C | 888G | 2251G | 2260A | 2413G | 2589A | 2754G | 3099T | 3304C | 3456G | 3573A | 3624G |
| 4162C | 4221G | 4230G | 4700G | 6580G | 6666G | | | | | | |
| 876C | 888A | 1980A | 2251A | 2260A | 2413G | 2589G | 2754G | 3099T | 3304C | 3456G | 3573A |
| 3624G | 4162C | 4221G | 4230G | 4700A | 6580G | 6666G | | | | | |
| 1980C | 2413A | 2589G | 2754G | 3573A | 3624G | 4162C | 4221G | 4230G | 4700A | 6580G | 6666G |
| 1980A | 2413A | 2589G | 2754G | 3573A | 3624G | 4162C | 4221G | 4230G | 4700A | 6580G | 6666G |
| 876C | 888A | 1980C | 2251G | 2260A | 2413G | 2589A | 2754G | 3099T | 3304C | 3456C | 3573A |
| 3624G | 4162C | 4221G | 4230T | 4700A | 6580G | 6666G | | | | | |
| 876C | 888A | 2251G | 2260A | 2413G | 2589A | 2754G | 3099T | 3304C | 3456G | 3573A | 3624G |
| 4162C | 4221G | 4230G | 4700G | 6580G | 6666G | | | | | | |
| 876C | 888G | 1980C | 2251G | 2260A | 2413G | 2589G | 2754A | 3099T | 3304C | 3456G | 3573A |
| 3624A | 4700A | 6580G | 6666G | | | | | | | | |
| 876C | 888G | 2251G | 2260A | 2413G | 2589A | 2754G | 3099T | 3304C | 3456G | 3573A | 3624G |
| 4162T | 4221G | 4230G | 4700G | 6580A | 6666G | | | | | | |
| 876C | 888A | 1980C | 2251G | 2260A | 2413G | 2589A | 2754G | 3099T | 3304T | 3456G | 3573A |
| 3624A | 4162C | 4221G | 4230G | 4700A | 6580G | 6666G | | | | | |
| 1980C | 2251G | 2260A | 2413G | 2589A | 2754G | 3099T | 3304C | 3456G | 3573A | 3624G | 4162C |
| 4221A | 4230G | 4700G | 6580G | 6666G | | | | | | | |
| 876C | 888A | 2251A | 2260A | 2413G | 2589A | 2754G | 3099T | 3304C | 3456G | 3573A | 3624G |
| 4221G | 6580G | 6666G | | | | | | | | | |
| 876C | 888A | 2251G | 2260A | 2413G | 2589A | 2754G | 3099T | 3304C | 3456G | 3573A | 3624G |
| 4162C | 4221G | 4230G | 4700A | | | | | | | | |
| 876C | 888G | 2251A | 2260A | 2413G | 2589A | 2754G | 3099T | 3304C | 3456G | 3573A | 3624G |
| 4221G | 6580G | 6666G | | | | | | | | | |
| 876C | 888G | 2251G | 2260A | 2413G | 2589G | 2754G | 3099T | 3304C | 3456G | 3573A | 3624G |
| 4162C | 4221G | 4230G | 4700G | 6580G | 6666G | | | | | | |
| 876T | 888G | 1980A | 2413A | 2589G | 2754G | 3099T | 3304C | 3456G | 4700G | 65800 | 6666G |
| 2251G | 2260A | 2413G | 2589A | 2754G | 3099T | 3304C | 3456G | 3573A | 3624G | 4700A | 6580G |
| 6666G | | | | | | | | | | | |
| 2251G | 2260A | 24l3G | 2589G | 2754G | 3099T | 3304C | 3456C | 3573G | 3624G | 4162C | 4221G |
| 4230G | 4700A | 6580G | 6666G | | | | | | | | |
| 876C | 888G | 2251G | 2260A | 2413G | 2589G | 2754G | 3099T | 3304C | 3456G | 3573A | 3624G |
| 4162C | 4221G | 4230G | 4700A | 6666G | | | | | | | |
| 876C | 888A | 1980C | 2251G | 2260A | 2413G | 2589A | 2754G | 3099T | 3304C | 3456C | 3573A |
| 3624G | 4162C | 4221G | 4230G | 4700A | 6580G | 6666G | | | | | |
| 876C | 888G | 2251G | 2260A | 2413G | 2589G | 2754G | 3099T | 3304T | 3456G | 3573A | 3624G |
| 4162C | 4221G | 4230G | 4700A | 6580G | 6666G | | | | | | |
| 876C | 888G | 1980A | 2413A | 2589G | 2754G | 3099T | 3304C | 3456G | 3573A | 36240 | 4162C |
| 4221G | 4230G | 4700G | 6666G | | | | | | | | |
| 876C | 888A | 1980C | 2251A | 2260A | 2413A | 2589A | 2754A | 3099T | 3304C | 3456C | 3573G |
| 3624G | 4162C | 4221G | 4230G | 4700G | 6580G 6666G | | | | | | |
| 2251G | 2260A | 2413G | 2589A | 2754G | 3099T | 3304C | 3456C | 3573A | 3624G | 4162C | 4221G |
| 4230G | 4700A | 6580G | 6666G | | | | | | | | |

| | | | |
|---|---|---|---|
| 876 | 756C > T | | Silent |
| 888 | 768G > A | Silent | |
| 1980 | 1860C > A | Silent | |
| 2251 | 2131G > A | V711M | |
| 2260 | 2l40A > C | T714P | |
| 2413 | 2293G > A | V765I | |
| 2589 | 2469A > G | I823M | |
| 2754 | 2634G > A | Silent | |
| 3099 | 2979T > G | Silent | |
| 3304 | 3184C > T | Silent | |
| 3456 | 3336G > C | E1112D | |
| 3573 | 3453A > G | Silent | |
| 3624 | 3504G > A | Silent | |
| 4162 | 4042C > T | L1348F | |
| 4221 | 4101G > A | Silent | |
| 4230 | 4110G > T | Q1370H | |
| 4700 | 4580G > A | R1527K | |
| 6580 | 6460G > A | D2154N | |
| 6666 | 6546G > A | Silent | |

| | | | | |
|---|---|---|---|---|
| M30795 | M30795 | 107910 | GEN-MVM | Aromatase (CYP19), promoter |

| | | |
|---|---|---|
| 747C | 825C | 881C |
| 747T | 825C | 881C |
| 747C | 825G | 881C |
| 747C | 825C | 881T |

| | | |
|---|---|---|
| 747 | 747C > T | Genomic |
| 825 | 825C > G | Genomic |
| 881 | 881C > T | Genomic |

| | | | | |
|---|---|---|---|---|
| AF044206 | AF044206 | 600262 | GEN-MVP | Homo sapiens cyclooxygenase |

(COX-2) gene, promoter and exon 1

| | | |
|---|---|---|
| 190 | 190T > C | Genomic |
| 270 | 270T > C | Genomic |
| 498 | 498A > G | Genomic |
| 534 | 534G > A | Genomic |
| 540 | 540C > T | Genomic |
| 684 | 684G > A | Genomic |
| 1177 | 1177A > G | Genomic |
| 1341 | 1341A > G | Genomic |
| 1381 | 1381T > C | Genomic |
| 1438 | 1438A > T | Genomic |
| 1648 | 1648T > C | Genomic |
| 1902 | 1902T > A | Genomic |
| 1904 | 1904G > T | Genomic |
| 2474 | 2474C > T | Genomic |
| 2486 | 2486G > C | Genomic |
| 2538 | 2538G > A | Genomic |
| 2615 | 2615A > G | Genomic |
| 2827 | 2827T > C | Genomic |
| 2926 | 2926T > G | Genomic |
| 3065 | 3065G > C | Genomic |
| 3141 | 3141C > T | Genomic |
| 3161 | 3161A > G | Genomic |
| 3658 | 3658T > G | Genomic |
| 3683 | 3683G > A | Genomic |
| 3739 | 3739C > A | Genomic |
| 3749 | 3749C > T | Genomic |
| 4295 | 4295A > C | Genomic |
| 4296 | 4296G > A | Genomic |
| 4816 | 4816C > A | Genomic |
| 4969 | 4969T > C | Genomic |
| 5402 | 5402A > G | Genomic |
| 5615 | 5615A > C | Genomic |
| 5990 | 5990A > G | Genomic |
| 6376 | 6376G > C | Genomic |
| 6978 | 6978C > G | Genomic |
| 7079 | 7079C > G | Genomic |

| | | | | |
|---|---|---|---|---|
| NM_006639 | NM_006639 | None | GEN-MVT | Homo sapiens cysteinyl |

leukotriene receptor 1 (CYSLT1) mRNA

| | | |
|---|---|---|
| 927 | 927C > T | Silent |

| | | | | |
|---|---|---|---|---|
| AL096870 | AL096870 | 601531 | GEN-MWO | Leukotriene B4 receptor, |

promoter and genomic

| | | |
|---|---|---|
| 399 | 399G > A | Genomic |
| 579 | 579A > G | Genomic |
| 755 | 755T > G | Genomic |
| 1191 | 1191A > G | Genomic |
| 1270 | 1270C > T | Genomic |
| 1874 | 1874A > C | Genomic |
| 1944 | 1944A > G | Genomic |
| 2107 | 2107C > T | Genomic |
| 2155 | 2155C > T | Genomic |
| 2383 | 2383G > A | Genomic |
| 3256 | 3256A > G | Genomic |
| 4250 | 4250G > A | Genomic |
| 4486 | 4486C > G | Genomic |
| 4753 | 4753C > G | Genomic |
| 5098 | 5098C > T | Genomic |
| 5209 | 5209A > T | Genomic |
| 5626 | 5626A > T | Genomic |
| 6314 | 6314A > C | Genomic |
| 7903 | 7903G > T | Genomic |
| 8032 | 8032G > A | Genomic |
| 8381 | 8381A > C | Genomic |
| 8573 | 8573A > C | Genomic |
| 9134 | 9134C > T | Genomic |
| 9224 | 9224G > A | Genomic |
| 9493 | 9493G > T | Genomic |
| 9524 | 9524G > A | Genomic |
| 10576 | 10576G > C | Genomic |
| 10756 | 10756T > G | Genomic |
| 11025 | 11025G > C | Genomic |
| 11163 | 11163T > C | Genomic |
| 11206 | 11206C > T | Genomic |
| 11398 | 11398C > A | Genomic |
| 11710 | 11710G > C | Genomic |
| 11766 | 11766G > T | Genomic |
| 11823 | 11823A > G | Genomic |
| 11948 | 11948A > G | Genomic |
| 12149 | 12149G > A | Genomic |

| | | | | |
|---|---|---|---|---|
| X02612 | X02612 | None | GEN-MW2 | Cytochrome P450 CYP1A1, promoter |

and genomic

| | | | | | | | | | | | |
|---|---|---|---|---|---|---|---|---|---|---|---|
| 546T | 547C | 573G | 588T | 658G | 1136G | 1987G | 3617C | 6305A | 6817C | 6819A | 6879G |
| 7597T | 7786C | | | | | | | | | | |
| 547C | 573G | 658G | 1136G | 1987G | 3617T | 6305A | 6817C | 6819A | 6879G | 7597C | 7786C |
| 547C | 573G | 658G | 1136G | 1987G | 3617T | 6305A | 6817C | 6819G | 6879G | 7597C | 7786C |
| 546C | 547C | 573G | 588T | 658G | 1136G | 3617C | 6305A | 6817C | 6819A | 6879G | 7597T |
| 7786A | | | | | | | | | | | |
| 546C | 547C | 573G | 588T | 658G | 1136G | 1987G | 3617C | 6305A | 6817C | 6819A | 6879G |
| 7597T | 7786C | | | | | | | | | | |
| 547C | 573G | 658G | 1136G | 1987G | 3617T | 6305A | 6817C | 6819A | 6879G | 7597T | 7786C |
| 547G | 573G | 658G | 1136G | 1987G | 3617T | 6305A | 6817C | 6819A | 6879G | 7597T | 7786A |
| 546C | 547C | 573G | 588T | 658G | 1136G | 1987T | 3617C | 6305A | 6817C | 6819A | 6879A |
| 7597T | 7786C | | | | | | | | | | |
| 546C | 547C | 573A | 1136G | 1987G | 6305A | 6817G | 6819A | 6879G | 7597T | 7786C | |
| 547C | 573G | 588G | 658G | 1136G | 1987G | 3617T | 6305A | 6817C | 6819G | 6879G | 7597T |
| 7786C | | | | | | | | | | | |
| 546C | 547C | 573G | 588T | 658G | 1136A | 3617C | 6305A | 6817C | 6819A | 6879G | 7597T |
| 7786A | | | | | | | | | | | |
| 546C | 547C | 573G | 588T | 658G | 1136G | 1987T | 3617C | 6305A | 6817C | 6819A | 6879G |
| 7597T | 7786C | | | | | | | | | | |
| 546C | 547C | 573G | 658G | 1136G | 6305G | | | | | | |
| 546C | 547C | 573G | 588G | 658G | 1136G | 1987G | 3617C | 6305A | 6817C | 6819A | 6879G |
| 7786C | | | | | | | | | | | |
| 547C | 573G | 588T | 658G | 1136G | 6305A | 6817C | 6819G | 6879G | 7597T | 7786C | |
| 547C | 573G | 658G | 1136G | 1987G | 3617T | 6305A | 6817C | 6819A | 6879G | 7597T | 7786A |
| 547C | 658G | 1136G | 6305A | 6817A | 6819A | 6879G | 7597T | 7786C | | | |
| 547C | 573G | 588G | 658G | 1136G | 1987G | 3617T | 6305A | 6817C | 6819G | 6879G | 7597T |
| 7786C | | | | | | | | | | | |
| 547C | 573G | 658G | 1136G | 1987G | 3617T | 6305A | 6817C | 6819G | 6879G | 7597C | 7786C |
| 546C | 547C | 573G | 588G | 658G | 1136G | 6305C | | | | | |
| 547C | 573G | 658G | 1136G | 1987G | 3617T | 6305A | 6817C | 6819A | 6879G | | |
| 547G | 573G | 658G | 1136G | 1987G | 3617T | 6305A | 6817C | 6819A | 6879G | 7597T | 7786A |
| 546C | 547C | 573G | 588G | 658G | 1136G | 1987G | 3617T | 6305A | 6817C | 6819G | 6879G |
| 546C | 547C | 573A | 588T | 658A | 1136G | 1987G | 3617T | 6305A | 6817C | 6819A | 6879G |
| 7597T | 7786C | | | | | | | | | | |
| 546C | 547C | 573G | 588T | 658G | 1136A | 1987T | 3617C | 6305A | 6817C | 6819A | 6879G |
| 7597T | 7786A | | | | | | | | | | |
| 546C | 547C | 573G | 588G | 658G | 1136G | 1987G | 3617T | 6305A | 6817C | 6819A | 6879G |
| 7597T | 7786C | | | | | | | | | | |
| 547C | 573G | 588T | 658G | 1136G | 1987G | 3617T | 6305A | 6817C | 6819A | 6879G | 7597T |
| 7786C | | | | | | | | | | | |
| 547C | 573G | 588T | 658G | 1136G | 1987G | 3617C | 6305A | 6817C | 6819A | 6879G | 7597T |
| 7786C | | | | | | | | | | | |
| 573G | 658G | 1136G | 1987G | 3617C | 6305A | 6817C | 6819A | 6879G | | | |
| 546C | 547C | 573G | 588G | 658G | 1136G | 1987G | 3617C | 6305A | 6817C | 6819A | 6879G |
| 7597C | 7786C | | | | | | | | | | |
| 547C | 573A | 588G | 658G | 1136G | 1987G | 6305A | 6817A | 6819A | 6879G | 7597T | 7786C |
| 547C | 573A | 588G | 658G | 1136G | 1987G | 6305A | 6817C | 6819A | 6879G | 7597T | 7786C |
| 547C | 573G | 588T | 658G | 1136G | 1987G | 3617T | 6305A | 6817C | 6819G | 6879G | 7597T |
| 7786C | | | | | | | | | | | |

| | | |
|---|---|---|
| 546 | 546C > T | Genomic |
| 547 | 547C > G | Genomic |
| 573 | 573G > A | Genomic |
| 588 | 588T > G | Genomic |
| 658 | 658G > A | Genomic |
| 1136 | 1136G > A | Genomic |
| 1987 | 1987T > G | Genomic |
| 3437 | 3437\hat{ }insC | Genomic |
| 3617 | 3617C > T | Genomic |
| 6305 | 6305A > C | Genomic |
| 6817 | 6817C > A | Genomic |
| 6819 | 6819G > A | Genomic |
| 6879 | 6879G > A | Genomic |
| 7597 | 7597T > C | Genomic |
| 7786 | 7786C > A | Genomic |

| | | | | |
|---|---|---|---|---|
| J02843 | J02843 | None | GEN-MW4 | Cytochrome P450 CYP2E1, promoter |

and genomic

| | | |
|---|---|---|
| 373 | 373T > G | Genomic |
| 582 | 582C > T | Genomic |
| 637 | 637C > G | Genomic |
| 646 | 646C > T | Genomic |
| 1051 | 1051C > T | Genomic |
| 1172 | 1172G > C | Genomic |
| 1179 | 1179A > G | Genomic |
| 1262 | 1262T > A | Genomic |
| 1312 | 1312T > G | Genomic |
| 1409 | 1409C > A | Genomic |
| 1435 | 1435T > G | Genomic |
| 1483 | 1483C > G | Genomic |
| 1532 | 1532G > C | Genomic |
| 1772 | 1772C > T | Genomic |
| 1800 | 1800T > C | Genomic |
| 1896 | 1896A > G | Genomic |
| 2019 | 2019T > C | Genomic |
| 2413 | 2413T > C | Genomic |
| 2473 | 2473A > G | Genomic |
| 2492 | 2492T > A | Genomic |
| 2754 | 2754G > T | Genomic |
| 3372 | 3372G > A | Genomic |
| 3811 | 3811C > A | Genomic |
| 3858 | 3858C > T | Genomic |
| 4182 | 4182T > C | Genomic |
| 4236 | 4236C > G | Genomic |
| 4504 | 4504C > T | Genomic |
| 4565 | 4565G > A | Genomic |
| 4574 | 4574G > A | Genomic |
| 4703 | 4703C > G | Genomic |
| 5155 | 5155G > A | Genomic |
| 5657 | 5657C > T | Genomic |
| 5737 | 5737C > T | Genomic |
| 5926 | 5926T > C | Genomic |
| 6103 | 6103C > T | Genomic |
| 6300 | 6300G > A | Genomic |
| 6418 | 6418G > A | Genomic |
| 6497 | 6497G > A | Genomic |
| 6609 | 6609C > T | Genomic |
| 6629 | 6629T > C | Genomic |
| 6741 | 6741T > A | Genomic |
| 6999 | 6999G > A | Genomic |
| 7257 | 7257C > T | Genomic |
| 7275 | 7275C > G | Genomic |
| 7310 | 7310G > T | Genomic |
| 7353 | 7353C > T | Genomic |
| 7520 | 7520G > A | Genomic |
| 7592 | 7592G > A | Genomic |
| 7669 | 7669T > C | Genomic |
| 7728 | 7728T > C | Genomic |
| 7915 | 7915C > T | Genomic |
| 7934 | 7934C > T | Genomic |
| 7935 | 7935A > G | Genomic |
| 8449 | 8449G > A | Genomic |
| 8526 | 8526C > T | Genomic |
| 8610 | 8610C > T | Genomic |
| 8619 | 8619A > G | Genomic |
| 8835 | 8835A > G | Genomic |
| 8841 | 8841C > A | Genomic |
| 8857 | 8857G > T | Genomic |
| 8873 | 8873G > A | Genomic |
| 9638 | 9638A > G | Genomic |
| 9787 | 9787T > C | Genomic |
| 9940 | 9940G > A | Genomic |
| 10101 | 10101T > C | Genomic |
| 10171 | 10171C > T | Genomic |
| 10456 | 10456T > A | Genomic |
| 10491 | 10491A > G | Genomic |
| 10622 | 10622C > T | Genomic |
| 10698 | 10698C > T | Genomic |
| 10869 | 10869C > A | Genomic |
| 11138 | 11138C > A | Genomic |
| 11195 | 11195A > G | Genomic |
| 11279 | 11279G > A | Genomic |
| 11449 | 11449G > A | Genomic |
| 12454 | 12454G > T | Genomic |
| 12569 | 12569C > T | Genomic |
| 12720 | 12720C > G | Genomic |
| 12757 | 12757A > T | Genomic |
| 12811 | 12811C > G | Genomic |
| 12945 | 12945C > T | Genomic |
| 13369 | 13369A > G | Genomic |
| 13593 | 13593T > C | Genomic |
| 13656 | 13656G > A | Genomic |
| 13680 | 13680C > T | Genomic |
| 13733 | 13733G > A | Genomic |
| 14070 | 14070C > T | Genomic |
| 14089 | 14089A > T | Genomic |
| 14094 | 14094G > A | Genomic |

| | | | | |
|---|---|---|---|---|
| X17059 | X17059 | None | GEN-MWB | N-acetyltransferase 1, genomic |

sequence (not including promoter)

| | | | | | | | |
|---|---|---|---|---|---|---|---|
| 163T | 401A | 405T | | | | | |
| 163T | 401A | 405A | 885G | 899G | 1000G | 1528A | 1535A |
| 163A | 401A | 885G | 899G | 1000G | 1528A | 1535A | |
| 163T | 401A | 405A | 885G | 899G | 1000A | 1528T | 1535C |
| 163T | 401A | 405A | 885G | 899G | 1000G | 1528T | 1535A |
| 163T | 401T | 405A | 1000G | 1528T | 1535A | | |
| 163T | 401A | 405A | 885G | 899G | 1000G | 1528T | 1535C |
| 163T | 401A | 405T | | | | | |
| 163A | 401A | 405T | 885G | 899G | 1000G | 1528A | 1535A |
| 163T | 401T | 405A | 885A | 899A | 1000G | 1528T | 1535A |

| | | |
|---|---|---|
| 163 | (−278)T > A | Genomic |
| 401 | (−40)A > T | Genomic |
| 405 | (−36)A > T | Genomic |
| 885 | 445G > A | Genomic |
| 899 | 459G > A | Genomic |
| 1000 | 560G > A | Genomic |
| 1528 | 1088T > A | Genomic |
| 1535 | 1095A > C | Genomic |

| | | | | |
|---|---|---|---|---|
| U22027 | U22027 | None | GEN-MWD | Cytochrome P450 CYP2A6, promoter |

and genomic

| | | | | | |
|---|---|---|---|---|---|
| 841G | 2936A | 5090G | 5262A | | |
| 841A | 934G | 2936G | 5090G | 5262G | |
| 841G | 934G | 2612C | 2936G | 5090G | 5262G |
| 841A | 934G | 2612C | 2936A | 5090G | 5262G |
| 841G | 934G | 2612C | 2936A | 5090G | 5262G |
| 841G | 934G | 2612A | 2936A | 5090G | 5262G |
| 841G | 934G | 2612C | 2936A | 5090A | 5262G |
| 841G | 934A | 2612C | 2936A | 5090G | 5262A |
| 841G | 2612C | 2936G | 5090G | 5262G | |
| 5090G | 5262G | | | | |
| 841G | 2612A | 2936A | 5090A | 5262G | |
| 841G | 934G | 2612C | 2936A | 5090A | 5262A |
| 2612C | 2936A | 5090G | 5262A | | |
| 841A | 934G | 2612C | 2936G | 5090G | 5262G |
| 841G | 934A | 2612A | 2936A | 5090G | 5262A |
| 841G | 2612A | 2936G | 5090G | 5262G | |
| 841A | 2612A | | | | |
| 841G | 934G | 2612C | 2936A | 5090G | 5262A |
| 841G | 934G | 2612A | 2936G | 5090G | 5262G |

| | | |
|---|---|---|
| 841 | 841G > A | Genomic |
| 934 | 934G > A | Genomic |
| 2612 | 2612C > A | Genomic |
| 2936 | 2936G > A | Genomic |
| 3900 | 3900A > C | Genomic |
| 4416 | 4416C > T | Genomic |
| 5090 | 5090G > A | Genomic |
| 5262 | 5262G > A | Genomic |

| | | | | |
|---|---|---|---|---|
| NM_004695 | NM_004695 | None | GEN-MWE | Homo sapiens solute carrier |

family 16 (monocarboxylic acid transporters), member 5 (SLC16A5) mRNA

| | | |
|---|---|---|
| 913 | 853A > G | I28EV |

| | | | | |
|---|---|---|---|---|
| NM_004211 | NM_004211 | None | GEN-MWG | Homo sapiens solute carrier |

family 6 (neurotransmitter transporter, glycine), member 5 (SLC6A5) mRNA

| | | |
|---|---|---|
| 2678 | 23970 > T | 3′ |

| | | | | |
|---|---|---|---|---|
| D13305 | D13305 | None | GEN-MWW | Human mRNA for brain |

cholecystokinin receptor

| | |
|---|---|
| 1945A | 1973G |
| 1945C | 1973G |
| 1945C | 1973A |
| 1945A | 1973A |

| | | |
|---|---|---|
| 1945 | 1753C > A | 3′ |
| 1973 | 1781A > G | 3′ |

| | | | | |
|---|---|---|---|---|
| NM_000966 | NM_000966 | None | GEN-MX8 | Homo sapiens retinoic acid |

receptor, gamma (RARG) mRNA

| | | | |
|---|---|---|---|
| 1426C | 1627A | 2202T | 2328G |
| 1426C | 1627A | 2202C | 2328C |
| 1426C | 1627G | 2202T | 2328C |
| 1426C | 1627A | 2202T | 2328C |
| 1426T | 2202T | 2328C | |
| 1426C | 2202T | 2328C | |
| 1426C | 1627G | 2202T | 2328G |

| | | |
|---|---|---|
| 1426 | 1280C > T | S427L |
| 1627 | 1481A > G | 3′ |
| 2202 | 2056T > C | 3′ |
| 2328 | 21820 > G | 3′ |

| | | | | |
|---|---|---|---|---|
| NM_000176 | NM_000176 | None | GEN-MXL | Homo sapiens nuclear receptor |

subfamily 3, group C, member 1 (NR3C1), mRNA

| | | | | | |
|---|---|---|---|---|---|
| 1220A | 1896C | 2430T | 3642C | 4346G | |
| 1220A | 1896T | 2430T | 3642C | 4346A | 4654A |
| 1220A | 1896C | 2430T | 3642C | 4346A | 4654A |
| 1220A | 1896C | 2430C | 3642C | 4346A | 4654A |
| 1220G | 1896C | 2430T | 3642C | 4346A | 4654A |
| 1220A | 1896C | 2430T | 3642T | 4346A | 4654A |
| 1220A | 1896C | 2430T | 3642C | 4346G | 4654G |

| | | |
|---|---|---|
| 1220 | 1088A > G | N363S |
| 1896 | 1764C > T | Silent |
| 2430 | 2298T > C | Silent |
| 3642 | 3510C > T | 3′ |
| 4346 | 4214A > G | 3′ |
| 4654 | 4522A > G | 3′ |

| | | | | |
|---|---|---|---|---|
| NM_000367 | NM_000367 | None | GEN-MXO | Homo sapiens thiopurine 5- |

methyltransferase (TPMT) mRNA

| | | |
|---|---|---|
| 784A | 1074T | 2108C |
| 784G | 1074T | 2108C |
| 784A | 1074T | 2108G |
| 784A | 1074C | 2108C |

| | | |
|---|---|---|
| 784 | 719A > G | Y240C |
| 1074 | 1009T > C | 3′ |
| 2108 | 2043C > G | 3′ |

| | | | | |
|---|---|---|---|---|
| NM_001085 | NM_001085 | None | GEN-MXS | Homo sapiens alpha-1- |

antichymotrypsin (AACT) mRNA

| | | |
|---|---|---|
| 401 | 390C > T | Silent |

| | | | | |
|---|---|---|---|---|
| NM_001045 | NM_001045 | None | GEN-MXT | Homo sapiens solute carrier |

family 6 (neurotransmitter transporter, serotonin), member 4 (SLC6A4) mRNA

| | | |
|---|---|---|
| 2293G | 2406T | 2428G |
| 2293A | 2406C | 2428G |
| 2293A | 2406T | 2428T |
| 2293A | 2406T | 2428G |

| | | |
|---|---|---|
| 2293 | 2221A > G | 3′ |
| 2406 | 2334T > C | 3′ |
| 2428 | 2356G > T | 3′ |

| | | | | |
|---|---|---|---|---|
| M83181 | M83181 | None | GEN-MY7 | Serotonin receptor 5HT-1A, |

complete cds

| | | |
|---|---|---|
| 661G | 685G | 778C |
| 661G | 685G | 778A |
| 661T | 685G | 778C |
| 661G | 685A | 778C |

| | | |
|---|---|---|
| 661 | 270G > T | Silent |
| 685 | 294G > A | Silent |
| 778 | 387C > A | Silent |

| | | | | |
|---|---|---|---|---|
| NM_000054 | NM_000054 | None | GEN-MYY | Homo sapiens arginine |

vasopressin receptor 2 (nephrogenic diabetes insipidus) (AVPR2) mRNA

| | | |
|---|---|---|
| 675C | 878G | 1162G |
| 675T | 878G | 1162G |
| 675C | 878A | 1162G |
| 675C | 878G | 1162A |

| | | |
|---|---|---|
| 675 | 440C > T | A147V |
| 878 | 643G > A | V215M |
| 1162 | 927A > G | Silent |

| | | | | |
|---|---|---|---|---|
| NM_001080 | NM_001080 | None | GEN-MZO | Homo sapiens Succinic |

semialdehyde dehydrogenase (SSADH) mPLNA

| | | |
|---|---|---|
| 1664 | 1664C > T | 3′ |
| 1861 | 1861C > A | 3′ |

| | | | | |
|---|---|---|---|---|
| AC000111 | AC000111 | None | GEN-MZM | Human BAC clone 068P20 from |

7q31-q32, complete sequence

| | | | | | | | | | | |
|---|---|---|---|---|---|---|---|---|---|---|
| 1287C | 1517A | 2330A | 2349G | 2406G | 2582G | 2616A | 3354G | 3383G | 3772C | 4283A |
| 1287C | 2349G | 2406A | 2582A | 2616A | 3383G | 4283T | | | | |
| 1287C | 1517A | 2349G | 2582G | 2616A | 3354C | 3383G | 3772C | 4283T | | |
| 1517A | 2349G | 2406G | 2582G | 2616A | 3772T | 4283T | | | | |
| 1287T | 1517A | 2330G | 2349G | 2406A | 2582G | 2616A | 3354C | 3383T | 4283T | |
| 1287C | 1517T | 2330A | 2349G | 2406A | 2582G | 3354G | 3383G | 3772C | 4283T | |
| 1287C | 2349G | 2406A | 2582G | 2616A | 3383G | 3772T | 4283T | | | |
| 1287C | 1517A | 2349A | 2582G | 2616A | 3383T | 3772C | 4283T | | | |
| 1287C | 1517A | 2330A | 2349G | 2406G | 2582G | 2616A | 3354G | 3383G | 3772C | 4283T |
| 1287C | 2330G | 2349G | 2582G | 2616A | 3354G | 3383G | 3772C | 4283T | | |
| 1287C | 1517T | 2330A | 2349G | 2406A | 2582G | 2616A | 3354G | 3383T | 3772C | 4283T |
| 1287C | 1517A | 2330A | 2349G | 2406A | 2582G | 2616A | 3354G | 3383T | 3772C | 4283T |
| 1287C | 1517A | 2330G | 2349G | 2406A | 2582G | 2616A | 3354C | 3383T | 3772T | 4283T |
| 1287C | 1517A | 2330G | 2349G | 2406A | 2582G | 2616A | 3354C | 3383T | 3772C | 4283T |
| 1287T | 1517A | 2349G | 2406G | 2582G | 2616A | 3772C | 4283T | | | |
| 1287C | 1517A | 2330A | 2349G | 2406A | 2582G | 2616A | 3354G | 3383G | 3772C | 4283T |
| 1287C | 1517A | 2330G | 2349G | 2406G | 2582G | 2616A | 3354G | 3383T | 3772C | 4283T |
| 1287C | 1517A | 2330G | 2349G | 2406G | 2582G | 2616A | 3354C | 3383T | 3772C | 4283T |
| 1287T | 1517A | 2330A | 2349G | 2406G | 2582G | 2616A | 3354G | 3383G | 3772T | 4283T |
| 1287C | 1517T | 2330A | 2349G | 2406A | 2582G | 2616A | 3354G | 3383G | 3772T | 4283T |
| 2330G | 2349G | 2406A | 2582G | 2616A | 3354C | 3383T | 3772T | 4283T | | |
| 1287C | 1517A | 2330G | 2349G | 2406G | 2582G | 2616A | 3354C | 3383T | 3772C | 4283A |
| 1287C | 1517A | 2330A | 2349G | 2406G | 2582G | 2616A | 3354C | 3383T | 3772C | 4283T |
| 1287C | 1517T | 2330G | 2349G | 2406G | 2582G | 2616A | 3354C | 3383G | 3772T | 4283T |
| 1287C | 1517T | 2330G | 2349G | 2406G | 2582G | 2616A | 3354G | 3383G | 3772C | 4283T |
| 1287C | 1517A | 2330G | 2349G | 2406G | 2582G | 2616A | 3354C | 3383G | 3772C | 4283T |
| 1517A | 2330G | 2349G | 2406A | 2582G | 3772T | | | | | |
| 1287C | 1517A | 2330A | 2349A | 2406G | 2582G | 2616A | 3354G | 3383T | 3772C | 4283T |
| 1287C | 1517T | 2330G | 2349G | 2406A | 2582A | 2616A | 3354C | 3383G | 3772T | 4283T |
| 2330G | 2349G | 3383G | 4283T | | | | | | | |
| 1287T | 1517A | 2330G | 2349G | 2406A | 2582G | 2616A | 3354C | 3383T | 3772C | 4283T |
| 1287C | 1517T | 2330A | 2349G | 2406A | 2582G | 2616G | 3354G | 3383G | 3772C | 4283T |

| 472 472-486delTTTGCTTCAAOAATA Genomic | | |
|---|---|---|
| 1287 | 1287C > T | Genomic |
| 1517 | 1517A > T | Genomic |
| 2330 | 2330A > G | Genomic |
| 2349 | 2349G > A | Genomic |
| 2406 | 2406G > A | Genomic |

| | | |
|---|---|---|
| 2519 | 2519-2521delCTT | Genomic |

| | | |
|---|---|---|
| 2582 | 2582G > A | Genomic |
| 2616 | 2616A > G | Genomic |
| 3354 | 3354G > C | Genomic |
| 3383 | 3383G > T | Genomic |
| 3772 | 3772C > T | Genomic |
| 4283 | 4283T > A | Genomic |

| | | | | |
|---|---|---|---|---|
| GEN-199-NT_001649_6 | NT_001649 | None | GEN-N08 | Multidrug resistance |

protein 3 exon 7

| | | |
|---|---|---|
| 84 | 84T > C | Genomic |

| | | | | |
|---|---|---|---|---|
| GEN-199-NT_001649_0 | NT_001649 | None | GEN-N0A | Multidrug resistance |

protein 3 exon 1

| | | |
|---|---|---|
| 311 | 311G > A | Genomic |

| | | | | |
|---|---|---|---|---|
| GEN-LV1-NT_000648_11 | NT_000648 | None | GEN-N0E | histidine |

decarboxylase (HDC) exon 12

| | | |
|---|---|---|
| 471 | 471T > C | Genomic |

| | | | | |
|---|---|---|---|---|
| GEN-4CS-NT_000568_11 | NT_000568 | None | GEN-N12 | prostate-specific |

membrane antigen (PSM) exon 12

| | | |
|---|---|---|
| 41 | 41T > G | Genomic |

| | | | | |
|---|---|---|---|---|
| GEN-2O6-NT_001987_0 | NT_001987 | None | GEN-N1R | Integral membrane |

protein (Nramp2) exon 1

| | | |
|---|---|---|
| 229C | 236C | 1176C |
| 229C | 236T | 1176A |
| 229C | 236C | 1176A |
| 229T | 236C | |
| 229T | 236C | 1176A |
| 229T | 236C | |

| | | |
|---|---|---|
| 229 | 229C > T | Genomic |
| 236 | 236C > T | Genomic |
| 1176 | 1176C > A | Genomic |

| | | | | |
|---|---|---|---|---|
| GEN-3L1-NT_000876_6 | NT_000876 | None | GEN-N1X | phospholipid |

hydroperoxide glutathione peroxidase exon 7

| | | | | |
|---|---|---|---|---|
| 38C | 117G | 176C | 293G | 380C |
| 38C | 117G | 176C | 293G | 380T |
| 38G | 117G | 176T | 293A | 380T |
| 38G | 117A | 293G | 380T | |
| 38G | 117G | 176T | 293G | 380T |
| 38G | 117G | 176C | 293G | 380T |
| 38G | 117A | 176C | 293G | 380T |

| | | |
|---|---|---|
| 38 | 38G > C | Genomic |
| 117 | 117G > A | Genomic |
| 176 | 176T > C | Genomic |
| 293 | 293G > A | Genomic |
| 380 | 380T > C | Genomic |

| | | | | |
|---|---|---|---|---|
| GEN-3L1-NT_000876_2 | NT_000876 | None | GEN-N20 | phospholipid |

hydroperoxide glutathione peroxidase exon 3

| | | |
|---|---|---|
| 45 | 45T > C | Genomic |

| | | | | |
|---|---|---|---|---|
| GEN-3L1-NT_000876_0 | NT_000876 | None | GEN-N22 | phospholipid |

hydroperoxide glutathione peroxidase exon 1

| | | | | | |
|---|---|---|---|---|---|
| 805A | 822C | 1008T | 1084G | 1115G | |
| 805A | 819A | 822C | 1008C | 1084G | 1115A |
| 805A | 822C | 1008C | 1084A | 1115G | |
| 805A | 819G | 822C | 1008C | 1084G | 1115G |
| 805T | 819A | 1008C | 1084G | 1115G | |
| 805A | 819A | 822C | 1008C | 1084G | 1115G |
| 805T | 819A | 822T | 1008C | 1084G | 1115G |
| 805A | 819G | 822C | 1008T | 1084G | 1115G |
| 819A | 1008T | 1084G | 1115G | | |
| 805A | 819G | 822C | 1008C | 1084A | 1115G |

| | | |
|---|---|---|
| 805 | 805A > T | Genomic |
| 819 | 819A > G | Genomic |
| 822 | 822C > T | Genomic |
| 1008 | 1008C > T | Genomic |
| 1084 | 1084G > A | Genomic |
| 1115 | 1115G > A | Genomic |

| | | | | |
|---|---|---|---|---|
| GEN-3L1-NT_000876_1 | NT_000876 | None | GEN-N23 | phospholipid |

hydroperoxide glutathione peroxidase exon 2

| | | |
|---|---|---|
| 225 | 225T > C | Genomic |

| | | | | |
|---|---|---|---|---|
| GEN-KV0-NT_000562_0 | NT_000562 | None | GEN-N2A | Tryptophan hydroxylase |
| exon 1 | | | | |

| | | |
|---|---|---|
| 827 | 827A > T | Genomic |

| | | | | |
|---|---|---|---|---|
| GEN-LV1-NT_000648_8 | NT_000648 | None | GEN-N2Y | histidine decarboxylase |
| (HDC) exon 9 | | | | |

| | | |
|---|---|---|
| 160 | 160T > C | Genomic |

| | | | | |
|---|---|---|---|---|
| GEN-LV1-NT_000648_2 | NT_000648 | None | GEN-N30 | histidine decarboxylase |
| (HDC) exon 3 | | | | |

| | | |
|---|---|---|
| 253 | 253T > C | Genomic |

| | | | | |
|---|---|---|---|---|
| GEN-LV-NT_000648_3 | NT_000648 | None | GEN-N31 | histidine decarboxylase |
| (HDC) exon 4 | | | | |

| | | |
|---|---|---|
| 256 | 256G > C | Genomic |

| | | | | |
|---|---|---|---|---|
| GEN-LV1-NT_000648_0 | NT_000648 | None | GEN-N32 | histidine decarboxylase |
| (HDC) exon 1 | | | | |

| | | |
|---|---|---|
| 1133 | 1133C > T | Genomic |

| | | | | |
|---|---|---|---|---|
| GEN-LV1-NT_000648_7 | NT_000648 | None | GEN-N35 | histidine decarboxylase |
| (HDC) exon 8 | | | | |

| | | |
|---|---|---|
| 200 | 200C > T | Genomic |

| | | | | |
|---|---|---|---|---|
| GEN-LV1-NT_000648_4 | NT_000648 | None | GEN-N36 | histidine decarboxylase |
| (HDC) exon 5 | | | | |

| | | |
|---|---|---|
| 287 | 287T > C | Genomic |

| | | | | |
|---|---|---|---|---|
| GEN-LU5-NT_001817_17 | NT_001817 | None | GEN-N3G | Cytosolic |

phospholipase A2 exon 18

| | |
|---|---|
| 446C | 587A |
| 446T | 587G |
| 446C | 587G |

| | | |
|---|---|---|
| 446 | 446C > T | Genomic |
| 587 | 587G > A | Genomic |

| | | | | |
|---|---|---|---|---|
| GEN-MJW-NT_002940_5 | NT_002940 | None | GEN-N3M | Chloride channel 5 exon |
| 6 (complement) | | | | |

| | | |
|---|---|---|
| 215 | 215C > T | Genomic |

| | | | | |
|---|---|---|---|---|
| GEN-MJW-NT_002940_0 | NT_002940 | None | GEN-N3P | Chloride channel 5 exon |
| 1 (complement) | | | | |

| | | |
|---|---|---|
| 218 | 218G > C | Genomic |

| | | | | |
|---|---|---|---|---|
| GEN-PS-NT_000840_8 | NT_000840 | None | GEN-N3S | myeloperoxidase exon 9 |
| (complement) | | | | |

| | | |
|---|---|---|
| 47 | 47C > T | Genomic |

| | | | | |
|---|---|---|---|---|
| GEN-PS-NT_000840_7 | NT_000840 | None | GEN-N3T | myeloperoxidase exon 8 |
| (complement) | | | | |

| | | |
|---|---|---|
| 81 | 81C > T | Genomic |

| | | | | |
|---|---|---|---|---|
| GEN-PS-NT_000840_1 | NT_000840 | None | GEN-N3Z | myeloperoxidase exon 2 |
| (complement) | | | | |

| | |
|---|---|
| 192C | 207A |
| 192A | 207G |
| 192C | 207G |
| 192A | 207A |

| | | |
|---|---|---|
| 192 | 192C > A | Genomic |
| 207 | 207G > A | Genomic |

| | | | | |
|---|---|---|---|---|
| GEN-PS-NT_000840_0 | NT_000840 | None | GEN-N40 | myeloperoxidase exon 1 |
| (complement) | | | | |

| | | | |
|---|---|---|---|
| 123C | 319A | 507C | 897C |
| 123C | 319A | 507T | 897T |
| 123C | 319A | 507T | 897C |
| 123T | 319A | 507T | 897T |
| 123C | 319G | 507T | |
| 123C | 319G | 507T | 897T |

| | | |
|---|---|---|
| 123 | 123C > T | Genomic |
| 319 | 319A > G | Genomic |
| 507 | 507T > C | Genomic |
| 897 | 897C > T | Genomic |

| | | | | |
|---|---|---|---|---|
| GEN-5Q-NT_000564_1 | NT_000564 | None | GEN-N48 | Cell surface receptor |

for sulfonylureas exon 2

| | | |
|---|---|---|
| 158 | 158T > C | Genomic |

| | | | | |
|---|---|---|---|---|
| GEN-5Q-NT_000564_3 | NT_000564 | None | GEN-N4A | Cell surface receptor |

for sulfonylureas exon 4

| | | |
|---|---|---|
| 280 | 280C > T | Genomic |

| | | | | |
|---|---|---|---|---|
| GEN-PS-NT_000840_11 | NT_000840 | None | GEN-N4D | myeloperoxidase exon 12 |
| (complement) | | | | |

| | | |
|---|---|---|
| 1005T | 1021G | 1129T |
| 1005T | 1021G | 1129G |
| 1005C | 1021G | 1129T |
| 1005T | 1021A | 1129T |

| | | |
|---|---|---|
| 1005 | 1005T > C | Genomic |
| 1021 | 1021G > A | Genomic |
| 1129 | 1129T > G | Genomic |

| | | | | |
|---|---|---|---|---|
| GEN-PS-NT_000840_10 | NT_000840 | None | GEN-N4E | myeloperoxidase exon 11 |
| (complement) | | | | |

| | | |
|---|---|---|
| 82 | 82G > A | Genomic |

| | | | | |
|---|---|---|---|---|
| GEN-LU5-NT_001817_8 | NT_001817 | None | GEN-N4X | Cytosolic phospholipase |
| A2 exon 9 | | | | |

| | | |
|---|---|---|
| 80 | 80G > A | Genomic |

| | | | | |
|---|---|---|---|---|
| GEN-2DF-NT_000476_26 | NT_000476 | None | GEN-N4Z | Cystic fibrosis exon |
| 27 | | | | |

| | | | |
|---|---|---|---|
| 1111A | 1594A | 1792A | |
| 1111A | 1551C | 1594G | 1792G |
| 1111A | 1551T | 1594G | 1792A |
| 1111G | 1551C | 1594G | 1792A |
| 1111A | 1551C | 1594G | 1792A |
| 1111A | 1551C | 1594A | 1792A |

| | | |
|---|---|---|
| 1111 | 1111A > G | Genomic |
| 1551 | 1551C > T | Genomic |
| 1594 | 1594G > A | Genomic |
| 1792 | 1792A > G | Genomic |

| | | | | |
|---|---|---|---|---|
| GEN-2DF-NT_000476_16 | NT_000476 | None | GEN-N59 | Cystic fibrosis exon |
| 17 | | | | |

| | | |
|---|---|---|
| 219 | 219T > C | Genomic |

| | | | | |
|---|---|---|---|---|
| GEN-2DF-NT_000476_10 | NT_000476 | None | GEN-N5F | Cystic fibrosis exon |
| 11 | | | | |

| | | |
|---|---|---|
| 115A | 291G | 325A |
| 115G | 291G | 325A |
| 115A | 291A | 325A |
| 115A | 291G | 325G |

| | | |
|---|---|---|
| 115 | 115G > A | Genomic |
| 291 | 291G > A | Genomic |
| 325 | 325A > G | Genomic |

| | | | | |
|---|---|---|---|---|
| GEN-106-NT_000744 _9 | NT_000744 | None | GEN-N5R | “Platelet-activating |

factor acetylhydrolase, isoform Ib, alpha subunit (45kD) exon 10”

| | |
|---|---|
| 190C | 1306A |
| 190T | 1306G |
| 190C | 1306G |

| | | |
|---|---|---|
| 190 | 190C > T | Genomic |
| 1306 | 1306A > G | Genomic |

| | | | | |
|---|---|---|---|---|
| GEN-106-NT_000744_4 | NT_000744 | None | GEN-N5U | “Platelet-activating |

factor acetylhydrolase, isoform Ib, alpha subunit (45kD) exon 5”

| | | |
|---|---|---|
| 295 | 295C > T | Genomic |

| | | | | |
|---|---|---|---|---|
| GEN-5Q-NT_000564_13 | NT_000564 | None | GEN-N6M | Cell surface receptor |

for sulfonylureas exon 14

| | | |
|---|---|---|
| 123 | 123G > A | Genomic |

| | | | | |
|---|---|---|---|---|
| GEN-5Q-NT_000564_17 | NT_000564 | None | GEN-N6Q | Cell surface receptor |

for sulfonylureas exon 18

| | |
|---|---|
| 35G | 50T |
| 35A | 50C |
| 35G | 50C |

| | | |
|---|---|---|
| 35 | 35G > A | Genomic |
| 50 | 50T > C | Genomic |

| | | | | |
|---|---|---|---|---|
| GEN-SQ-NT_000564_15 | NT_000564 | None | GEN-N6S | Cell surface receptor |

for sulfonylureas exon 16

| | | |
|---|---|---|
| 97 | 97C > T | Genomic |

| | | | | |
|---|---|---|---|---|
| GEN-SQ-NT_000564_22 | NT_000564 | None | GEN-N6X | Cell surface receptor |

for sulfonylureas exon 23

| | | |
|---|---|---|
| 292 | 292C > T | Genomic |

| | | | | |
|---|---|---|---|---|
| GEN-SQ-NT_000564_32 | NT_000564 | None | GEN-N77 | Cell surface receptor |

for sulfonylureas exon 33

| | | |
|---|---|---|
| 216 | 216G > T | Genomic |

| | | | | |
|---|---|---|---|---|
| GEN-SQ-NT_000564_30 | NT_000564 | None | GEN-N79 | Cell surface receptor |

for sulfonylureas exon 31

| | | |
|---|---|---|
| 162 | 162G > A | Genomic |

| | | | | |
|---|---|---|---|---|
| GEN-SQ-NT_000564_37 | NT_000564 | None | GEN-N7A | Cell surface receptor |

for sulfonylureas exon 38

| | | |
|---|---|---|
| 216 | 216G > C | Genomic |

| | | | | |
|---|---|---|---|---|
| GEN-3DE-NT_001504_0 | NT_001504 | None | GEN-N7W | platelet activating |

factor acetylhydrolase IB gamma-subunit exon 1 (compliment)

| | | |
|---|---|---|
| 483 | 483G > C | Genomic |

| | | | | |
|---|---|---|---|---|
| GEN-2DF-NT_000476_3 | NT_000476 | None | GEN-N89 | Cystic fibrosis exon 4 |

| | | |
|---|---|---|
| 184 | 184G > A | Genomic |

| | | | | |
|---|---|---|---|---|
| GEN-2DF-NT_000476_6 | NT_000476 | None | GEN-N8C | Cystic fibrosis exon 7 |

| | | |
|---|---|---|
| 236 | 236C > T | Genomic |

| | | | | |
|---|---|---|---|---|
| GEN-2O6-NT_001987_11 | NT_001987 | None | GEN-N8O | Integral membrane |

protein (Nramp2) exon 12

| | | |
|---|---|---|
| 82 | 82T > A | Genomic |

| | | | | |
|---|---|---|---|---|
| GEN-3S-NT_001039_3 | NT_001039 | None | GEN-N8W | Catechol-O |

methyltransferase exon 4

| | | | |
|---|---|---|---|
| 183T | 211G | 216G | 296A |
| 183C | 211G | 216G | 296G |
| 183C | 211G | 216A | 296G |
| 183T | 211G | 216G | 296G |
| 211T | 216G | 296G | |
| 183C | 211T | 216G | 296G |

| | | |
|---|---|---|
| 183 | 183T > C | Genomic |
| 211 | 211G > T | Genomic |
| 216 | 216G > A | Genomic |
| 296 | 296G > A | Genomic |

| | | | | |
|---|---|---|---|---|
| GEN-4CS-NT_000568_9 | NT_000568 | None | GEN-N8Y | prostate-specific |

membrane antigen (PSM) exon 10

| | |
|---|---|
| 38G | 234T |
| 38A | 234C |
| 38G | 234C |
| 38A | 234T |

| | | |
|---|---|---|
| 38 | 38G > A | Genomic |
| 234 | 234T > C | Genomic |

| | | | | |
|---|---|---|---|---|
| GEN-4CS-NT_000568_0 | NT_000568 | None | GEN-N91 | prostate-specific |

membrane antigen (PSM) exon 1

| | | |
|---|---|---|
| 195A | 476C | 595G |
| 195A | 476C | 595A |
| 195G | 476T | 595A |
| 195G | 476C | 595A |
| 476C | 595A | |

| | | |
|---|---|---|
| 195 | 195A > G | Genomic |
| 476 | 476C > T | Genomic |
| 595 | 595A > G | Genomic |

| | | | | |
|---|---|---|---|---|
| GEN-4CS-NT_000568_7 | NT_000568 | None | GEN-N94 | prostate-specific |

membrane antigen (PSM) exon 8

| | | |
|---|---|---|
| 84 | 84T > G | Genomic |
| 214 | 214T > C | Genomic |

| | | | | |
|---|---|---|---|---|
| GEN-4CS-NT_000568_5 | NT_000568 | None | GEN-N96 | prostate-specific |

membraneantigen (PSM) exon 6

| | | |
|---|---|---|
| 147A | 192C | 193G |
| 147C | 192T | 193G |
| 147C | l92C | 193G |
| 147A | 192C | 193A |

| | | |
|---|---|---|
| 147 | 147C > A | Genomic |
| 192 | 192C > T | Genomic |
| 193 | 193G > A | Genomic |

| | | | | |
|---|---|---|---|---|
| GEN-3MB-NT_001220_1 | NT_001220 | None | GEN-N9M | short-chain alcohol |

dehydrogenase (XH98G2) exon 2

| | | |
|---|---|---|
| 791 | 791G > C | Genomic |

| | | | | |
|---|---|---|---|---|
| GEN-21W-AC068647_4 | AC068647 | None | GEN-N16 | Homo sapiens chromosome |

3 clone RP11-64D22, WORKING DRAFT SEQUENCE, 8 unordered pieces

| | | |
|---|---|---|
| 334 | 334C > T | Genomic |
| 774 | 774C > T | Genomic |

| | | | | |
|---|---|---|---|---|
| GEN-1P5-AL356796_0 | AL356796 | None | GEN-NKT | Homo sapiens chromosome |

9 clone RP11-82I1, *** SEQUENCING IN PROGRESS ***, 28 unordered pieces

| | | |
|---|---|---|
| 723 | 723A > G | Genomic |
| 773 | 773T > A | Genomic |

| | | | | |
|---|---|---|---|---|
| GEN-GO-AC006994_3 | AC006994 | None | GEN-O1O | Homo sapiens BAC clone |

RP11-396J8 from 2, complete sequence

| | | |
|---|---|---|
| 173 | 173G > A | Genomic |

| | | | | |
|---|---|---|---|---|
| GEN-1MN-L10641_3 | L10641 | None | GEN-O2H | Human vitamin D-binding |

protein (GC) gene, complete cds

| | | |
|---|---|---|
| 315 | 315C > T | Genomic |

| | | | | |
|---|---|---|---|---|
| GEN-1MN-L10641_0 | L10641 | None | GEN-O2I | Human vitamin D-binding |

protein (GC) gene, complete cds

| | | |
|---|---|---|
| 238 | 1238C > T | Genomic |

| | | | | |
|---|---|---|---|---|
| GEN-1MN-L10641_1 | L10641 | None | GEN-O2J | Human vitamin D-binding |

protein (GC) gene, complete cds

| | | | |
|---|---|---|---|
| 1359 | 1359C > G | | Genomic |
| 1733 | 1733C > T | Genomic | |
| 1755 | 1755G > A | Genomic | |

| | | | | |
|---|---|---|---|---|
| GEN-1MN-L10641_6 | L10641 | None | GEN-O2K | Human vitamin D-binding |

protein (GC) gene, complete cds

| | | |
|---|---|---|
| 169 | 169A > G | Genomic |

| | | | | |
|---|---|---|---|---|
| GEN-1MN-L10641_7 | L10641 | None | GEN-O2L | Human vitamin D-binding |

protein (GC) gene, complete cds

| | | |
|---|---|---|
| 146 | 146A > G | Genomic |

| | | | | |
|---|---|---|---|---|
| GEN-1MN-L10641_4 | L10641 | None | GEN-O2M | Human vitamin D-binding |

protein (GC) gene, complete cds

| | | |
|---|---|---|
| 193 | 193A > G | Genomic |
| 434 | 434T > C | Genomic |

| | | | | |
|---|---|---|---|---|
| GEN-1MN-L10641_9 | L10641 | None | GEN-O2P | Human vitamin D-binding |

protein (GC) gene, complete cds

| | | |
|---|---|---|
| 455 | 455T > G | Genomic |

| | | | | |
|---|---|---|---|---|
| GEN-3B-AC003982_1 | AC003982 | None | GEN-O5V | Homo sapiens PAC clone |

166H1 from 12q, complete sequence

| | | |
|---|---|---|
| 240 | 240C > T | Genomic |

| | | | | |
|---|---|---|---|---|
| GEN-22Q-AC024085_10 | AC024085 | None | GEN-O5W | Human Chromosome 7 |

clone RP11-190G13, complete sequence

| | | |
|---|---|---|
| 472 | 472A > G | Genomic |

| | | | | |
|---|---|---|---|---|
| GEN-1ET-AP002027_1 | AP002027 | None | GEN-OTL | Homo sapiens genomic |

DNA, chromosome 4q22-q24, clone:496L13, complete sequence

| | | |
|---|---|---|
| 337 | 337G > A | Genomic |

| | | | | |
|---|---|---|---|---|
| GEN-3B6-AC008063_4 | AC008063 | None | GEN-OU5 | Homo sapiens BAC clone |

RP11-178A14 from 2, complete sequence

| | | |
|---|---|---|
| 170 | 170A > G | Genomic |

| | | | | |
|---|---|---|---|---|
| GEN-3B6-AC008063_1 | AC008063 | None | GEN-OUA | Homo sapiens BAC clone |

RP11-178A14 from 2, complete sequence

| | | |
|---|---|---|
| 417 | 417C > A | Genomic |

| | | | | |
|---|---|---|---|---|
| GEN-3AX-AC005006_8 | AC005006 | None | GEN-OUO | Homo sapiens clone RP1- |

56J10, complete sequence

| | | |
|---|---|---|
| 243 | 243C > T | Genomic |
| 288 | 288C > T | Genomic |
| 605 | 605C > T | Genomic |

| | | | | |
|---|---|---|---|---|
| GEN-MQ1-AL021068_0 | AL021068 | None | GEN-OXZ | Homo sapiens DNA |

sequence from PAC 206D15 on chromosome 1q24. contains a Reduced Folate Carrier
protein (RFC) LIKE gene, a mitochondrial ATP Synthetase protein 8 (ATP8, MTATP8)
LIKE pseudogene, an unknown gene and the last exon of the JEM1 gene coding for

| | | |
|---|---|---|
| 179 | 179T > C | Genomic |
| 243 | 243G > T | Genomic |
| 680 | 680T > C | Genomic |
| 790 | 790C > G | Genomic |

| | | | | |
|---|---|---|---|---|
| GEN-MQY-AL122002_5 | AL122002 | None | GEN-OY2 | Human DNA sequence from |

clone RP4-651E10 on chromosome 1p22.3-31.1, complete sequence

| | | |
|---|---|---|
| 45 | 45C > A | Genomic |
| 273 | 273A > T | Genomic |
| 467 | 467T > C | Genomic |

| | | | | |
|---|---|---|---|---|
| GEN-MQY-AL122002_7 | AL122002 | None | GEN-OY4 | Human DNA sequence from |

clone RP4-651E10 on chromosome 1p22.3-31.1, complete sequence

| | | |
|---|---|---|
| 276 | 276A > G | Genomic |

| | | | | |
|---|---|---|---|---|
| GEN-MQY-AL122002_3 | AL122002 | None | GEN-OY8 | Human DNA sequence from |

clone RP4-651E10 on chromosome 1p22.3-31.1, complete sequence

| | | |
|---|---|---|
| 179 | 179G > A | Genomic |

| | | | | |
|---|---|---|---|---|
| GEN-PB-Z84814_0 | Z84814 | None | GEN-OZA | Human DNA sequence from PAC |

172K2 on chromosome 6 contains HLA CLASS II DRA pseudogene, DRB3*01012 genes,
DRB9 pseudogene butyrophilin precursor and ESTs

| | | |
|---|---|---|
| 2093 | 2093A > G | Genomic |

| | | | | |
|---|---|---|---|---|
| GEN-PB-Z84814_1 | Z84814 | None | GEN-OZB | Human DNA sequence from PAC |

172K2 on chromosome 6 contains HLA CLASS II DRA pseudogene, DRB3*01012 genes,
DRB9 pseudogene butyrophilin precursor and ESTs

| | | |
|---|---|---|
| 361 | 361G > A | Genomic |
| 434 | 434G > A | Genomic |
| 618 | 618T > C | Genomic |
| 727 | 727C > T | Genomic |
| 784 | 784A > G | Genomic |
| 790 | 790G > A | Genomic |
| 874 | 874G > T | Genomic |

| | | | | |
|---|---|---|---|---|
| GEN-PB-Z84814_2 | Z84814 | None | GEN-OZC | Human DNA sequence from PAC |

172K2 on chromosome 6 contains HLA CLASS II DRA pseudogene, DRB3*01012 genes,
DRB9 pseudogene butyrophilin precursor and ESTs

| | | |
|---|---|---|
| 165 | 165T > C | Genomic |

| | | | | |
|---|---|---|---|---|
| GEN-QB-AC034228_0 | AC034228 | None | GEN-OZF | Homo sapiens chromosome 5 |

clone CTD-2198K16, WORKING DRAFT SEQUENCE, 19 unordered pieces

| | | |
|---|---|---|
| 1228 | 1228T > G | Genomic |
| 1608 | 1608C > T | Genomic |
| 1728 | 1728G > A | Genomic |
| 2396 | 2396A > G | Genomic |
| 2469 | 2469A > G | Genomic |

| | | | | |
|---|---|---|---|---|
| GEN-KYP-AL356218_1 | AL356218 | None | GEN-P1K | Homo sapiens chromosome |

9 clone RP11-311H10, *** SEQUENCING IN PROGRESS ***, 20 unordered pieces

| | | |
|---|---|---|
| 102 | 102C > G | Genomic |

| | | | | |
|---|---|---|---|---|
| GEN-9K-L44140_3 | L44140 | None | GEN-P22 | Homo sapiens chromosome X |

region from filamin (FLN) gene to glucose-6-phosphate dehydrogenase (G6PD) gene,
complete cds's

| | | |
|---|---|---|
| 276 | 276C > T | Genomic |

| | | | | |
|---|---|---|---|---|
| GEN-9K-L44140_5 | L44140 | None | GEN-P24 | Homo sapiens chromosome X |

region from filamin (FLN) gene to glucose-6-phosphate dehydrogenase (G6PD) gene,
complete cds's

| | | |
|---|---|---|
| 1149 | 1149G > A | Genomic |

| | | | | |
|---|---|---|---|---|
| GEN-EY-AC003043_0 | AC003043 | None | GEN-P92 | Homo sapiens chromosome |

17, clone HRPC1067MG, complete sequence

| | | |
|---|---|---|
| 1488 | 1488T > C | Genomic |

| | | | | |
|---|---|---|---|---|
| GEN-PH-AL132708_1 | AL132708 | None | GEN-PM6 | Human chromosome 14 DNA |

sequence *** IN PROGRESS *** BAC R-34911 of library RPCI-11 from chromosome 14
of Homo sapiens (Human), complete sequence

| | | |
|---|---|---|
| 1632 | 1632C > T | Genomic |

| | | | | |
|---|---|---|---|---|
| GEN-2KB-Z80898_4 | Z80898 | None | GEN-PND | Human DNA sequence from |

cosmid E1448 on chromosome 6p21.3 contains NRC class II HLA-DQB1

| | | |
|---|---|---|
| 73 | 73C > T | Genomic |
| 114 | 114G > A | Genomic |

| | | | | |
|---|---|---|---|---|
| GEN-2KB-Z80898_0 | Z80898 | None | GEN-PNE | Human DNA sequence from |

cosmid E1448 on chromosome 6p21.3 contains NRC class II HLA-DQB1

| | | |
|---|---|---|
| 207 | 207T > C | Genomic |

| | | | | |
|---|---|---|---|---|
| GEN-2KB-Z80898_3 | Z80898 | None | GEN-PNH | Human DNA sequence from |

cosmid E1448 on chromosome 6p21.3 contains MHC class II HLA-DQB1

| | | |
|---|---|---|
| 371 | 371C > A | Genomic |

| | | | | |
|---|---|---|---|---|
| GEN-KV6-AP001725_1 | AP001725 | None | GEN-PNI | Homo sapiens genomic |

DNA, chromosome 21q, section 69/105

| | | |
|---|---|---|
| 93 | 93C > T | Genomic |
| 180 | 180A > G | Genomic |
| 352 | 352G > A | Genomic |

| | | | | |
|---|---|---|---|---|
| GEN-9J-AC011780_0 | AC011780 | None | GEN-PPJ | Homo sapiens clone RP11 |

15H8, WORKING DRAFT SEQUENCE, 31 unordered pieces

| | | |
|---|---|---|
| 421 | 421A > G | Genomic |
| 702 | 702T > C | Genomic |

| | | | | |
|---|---|---|---|---|
| GEN-9J-AC011780_6 | AC011780 | None | GEN-PPL | Homo sapiens clone RP11- |

15H8, WORKING DRAFT SEQUENCE, 31 unordered pieces

| | | |
|---|---|---|
| 299 | 299C > T | Genomic |
| 303 | 303T > C | Genomic |

| | | | | |
|---|---|---|---|---|
| GEN-4R-AL133553_10 | AL133553 | None | GEN-PQM | Human DNA sequence from |

clone GS1-174L6 on chromosome 1, complete sequence

| | | |
|---|---|---|
| 175 | 175A > G | Genomic |
| 362 | 362C > T | Genomic |
| 365 | 365G > A | Genomic |

| | | | | |
|---|---|---|---|---|
| GEN-170-AL158847_2 | AL158847 | None | GEN-PQR | Homo sapiens chromosome |

1 clone RP4-735C1, *** SEQUENCING IN PROGRESS ***, 20 unordered pieces

| | | |
|---|---|---|
| 417 | 417C > T | Genomic |

| | | | | |
|---|---|---|---|---|
| GEN-1QL-AL157871_2 | AC011450 | None | GEN-PRN | Homo sapiens chromosome |

19 clone CTC-30107, complete sequence

| | | |
|---|---|---|
| 99 | 99G > C | Genomic |

| | | | | |
|---|---|---|---|---|
| GEN-25R-AC018988_7 | AC018988 | None | GEN-PS1 | Homo sapiens chromosome |

15 clone RP11-233C13 map 15, WORKING DRAFT SEQUENCE, 23 unordered pieces

| | | |
|---|---|---|
| 335 | 335G > A | Genomic |
| 367 | 367G > C | Genomic |

| | | | | |
|---|---|---|---|---|
| GEN-25R-AC018988_5 | AC018988 | None | GEN-PS3 | Homo sapiens chromosome |

15 clone RP11-233C13 map 15, WORKING DRAFT SEQUENCE, 23 unordered pieces

| | | |
|---|---|---|
| 115 | 115G > A | Genomic |
| 121 | 121A > C | Genomic |
| 346 | 346G > C | Genomic |

| | | | | |
|---|---|---|---|---|
| GEN-25R-AC018988_2 | AC018988 | None | GEN-PS4 | Homo sapiens chromosome |

15 clone RP11-233C13 map 15, WORKING DRAFT SEQUENCE, 23 unordered pieces

| | | |
|---|---|---|
| 1104 | 1104G > A | Genomic |
| 1111 | 1111T > C | Genomic |

| | | | | |
|---|---|---|---|---|
| GEN-2SV-AL096870_0 | AL096870 | None | GEN-Q9E | Human chromosome 14 DNA |

sequence *** IN PROGRESS *** BAC R-93459 of library RPCI-11 from chromosome 14
of Homo sapiens (Human), complete sequence

| | | |
|---|---|---|
| 932 | 932G > C | Genomic |

| | | | | |
|---|---|---|---|---|
| GEN-3G-AC013599_1 | AC013599 | None | GEN-QAN | Homo sapiens clone RP11- |

9N17, WORKING DRAFT SEQUENCE, 27 unordered pieces

| | | |
|---|---|---|
| 168 | 168T > C | Genomic |
| 191 | 191G > A | Genomic |
| 222 | 222G > T | Genomic |

| | | | | |
|---|---|---|---|---|
| GEN-1HZ--AC005519_7 | AC005519 | None | GEN-QBH | Homo sapiens PAC clone |

RP5-919J22 from 14q24.3, complete sequence

| | | |
|---|---|---|
| 180 | 150T > C | Genomic |

TABLE 4

| | | | | |
|---|---|---|---|---|
| AAC2 | D90040 | 243400 | GEN-465 | Human mRNA for |

arylamine N-acetyltransferase (EC 2.3.1.5)

| | | |
|---|---|---|
| 231 | 190C>T | R64W |
| 232 | 191G>A | R64Q |
| 382 | 341T>C | I114T |
| 522 | 481C>T | Silent |
| 631 | 590G>A | R197Q |
| 844 | 803A>G | K268R |
| 898 | 857A>G | E286G |
| 1062 | 1021T>C | 3′ |

| | | | | |
|---|---|---|---|---|
| AB000410 | AB000410 | 601982 | GEN-9O | Human hOGG1 |

mRNA, complete cds

| | | |
|---|---|---|
| 246 | (−23)A>G | 5′ |
| 251 | (−18)G>T | 5′ |
| 562 | 294G>A | Silent |

| | | | | |
|---|---|---|---|---|
| AB005659 | AB005659 | None | GEN-VR | Homo sapiens SMRP |

mRNA, complete cds

| | | |
|---|---|---|
| 4820 | 4084G>A | 3′ |

| | | | | |
|---|---|---|---|---|
| AB017546 | AB017546 | 601791 | GEN-L3J | Homo sapiens Pex14 |

mRNA for peroxisomal membrane anchor protein, complete cds

| | | |
|---|---|---|
| 161 | 156T>C | Silent |

| | | | | |
|---|---|---|---|---|
| ADH3 | M12272 | 103730 | GEN-1LU | Homo sapiens alcohol |

dehydrogenase class I gamma subunit (ADH3) mRNA, complete cds

| | | |
|---|---|---|
| 1128 | 1048A>G | I350V |

| | | | | |
|---|---|---|---|---|
| AF001437 | AF001437 | 245349 | GEN-9T | Dihydrolipoamide S- |

acetyltransferase (E2 component of pyruvate dehydrogenase complex)

| | | |
|---|---|---|
| 2000 | 1992G>T | 3′ |

| | | | | |
|---|---|---|---|---|
| AF001945 | AF001945 | 601691 | GEN-17Z | Homo sapiens rim |

ABC transporter (ABCR) mRNA, complete cds

| | | |
|---|---|---|
| 1492 | 1411G>A | E471K |
| 2336 | 2255G>T | S752I |
| 2646 | 2565G>A | Frame |
| 2669 | 2588G>C | G863A |
| 2872 | 2791G>A | V931M |
| 3164 | 3083C>T | A1028V |
| 3187 | 3106G>A | E1036K |
| 3292 | 3211ˆ 3212insGT | Frame |
| 4284 | 4203C>A | Silent |
| 4364 | 4283C>T | T1428M |
| 4813 | 4732G>A | G1578R |
| 5684 | 5603A>T | N18681 |
| 5763 | 5682G>C | Silent |
| 5963 | 5882G>A | G1961E |
| 6160 | 6079C>T | L2027F |
| 6229 | 6148G>C | V2050L |
| 6330 | 6249C>T | Silent |
| 6366 | 6285T>C | Silent |
| 6610 | 6529G>A | D2177N |
| 6774 | 6693C>T | Silent |

| | | | | |
|---|---|---|---|---|
| AF007216 | AF007216 | 603345 | GEN-13L | Homo sapiens sodium |

bicarbonate cotransporter (HNBC1) mRNA, complete cds

| | | |
|---|---|---|
| 1043 | 894A>C | R298S |
| 1678 | 1529G>A | R510H |

| | | | | |
|---|---|---|---|---|
| AF009746 | AF009746 | 603214 | GEN-1HZ | Homo sapiens |

peroxisomal membrane protein 69 (PMP69) mRNA, complete cds

| | | |
|---|---|---|
| 2060 | 2009T>C | 3′ |

| | | | | |
|---|---|---|---|---|
| AF027302 | AF027302 | 603429 | GEN-27T | Homo sapiens TNF- |

alpha stimulated ABC protein (ABC50) mRNA, complete cds

| | | |
|---|---|---|
| 3075 | 2981T>C | 3′ |

| | | | | |
|---|---|---|---|---|
| AF038007 | AF038007 | 602397 | GEN-2QG | Homo sapiens P-type |

ATPase FIC1 mRNA, partial cds

| | | |
|---|---|---|
| 941 | 941G>T | G314V |

| | | | | |
|---|---|---|---|---|
| AF044206 | AF044206 | 600262 | GEN-MVP | Homo sapiens |

cyclooxygenase (COX-2) gene, promoter and exon 1

| | | |
|---|---|---|
| 6978 | 6978C>G | Genomic |
| 7150 | 7150T>G | Genomic |

| | | | | |
|---|---|---|---|---|
| AF055025 | AF055025 | 300095 | GEN-32U | Homo sapiens clone |

24776 mRNA sequence

| | | |
|---|---|---|
| 1579 | 1579A>G | 3′ |

| | | | | |
|---|---|---|---|---|
| AF058921 | AF058921 | 603602 | GEN-LJY | Homo sapiens |

cytosolic phospholipase A2-gamma mRNA, complete cds

| | | |
|---|---|---|
| 1989 | 1680A>T | 3′ |

| | | | | |
|---|---|---|---|---|
| AF185589 | AF185589 | 124010 | GEN-MVA | Homo sapiens |

cytochrome P450 3A4 (CYP3A4) gene, promoter region

| | | |
|---|---|---|
| 10282 | 10282A>G | Genomic |

| | | | | |
|---|---|---|---|---|
| AJ001838 | AJ001838 | 603758 | GEN-17S | Homo sapiens mRNA |

for maleylacetoacetate isomerase

| | | |
|---|---|---|
| 197 | 94A>G | K32E |

| | | | | |
|---|---|---|---|---|
| ARSB | M32373 | 253200 | GEN-2J0 | Human arylsulfatase B |

(ASB) mRNA, complete cds

| | | |
|---|---|---|
| 182 | (−378)G>C | 5′ |
| 182 | (−378)G>T | 5′ |

| | | | | |
|---|---|---|---|---|
| ATM | U26455 | 208900 | GEN-2AT | Human |

phosphatidylinositol 3-kinase homolog (ATM) mRNA, complete cds

| | | |
|---|---|---|
| 1286 | 1027A>C | S343R |
| 1772 | 1513G>A | D505N |
| 1773 | 1514A>T | D505V |
| 2450 | 2191G>A | V731I |
| 3075 | 2816G>C | G939A |

| | | | | |
|---|---|---|---|---|
| ATP1A1 | D00099 | 182310 | GEN-4E8 | Homo sapiens mRNA |

for Na,K-ATPase alpha-subunit, complete cds

| | | |
|---|---|---|
| 3375 | 3057G>A | Silent |

| | | | | |
|---|---|---|---|---|
| AVPR2 | AF030626 | 304800 | GEN-2LO | Vasopressin receptor |

V2

| | | |
|---|---|---|
| 137 | 105G>A | Silent |
| 157 | 125C>T | A42V |
| 212 | 180G>T | Silent |
| 472 | 440C>T | A147V |
| 1025 | 993C>T | Silent |
| 1145 | 1113G>A | Silent |
| 1165 | 1133T>C | 3′ |

| | | | | |
|---|---|---|---|---|
| CAT | X04076 | 115500 | GEN-13P | Human kidney mRNA |

for catalase

| | | |
|---|---|---|
| 51 | (−20)T>C | 5′ |
| 1325 | 1255C>T | Silent |

| | | | | |
|---|---|---|---|---|
| CBG | J02943 | 122500 | GEN-Y2 | Human corticosteroid |

binding globulin mRNA, complete cds

| | | |
|---|---|---|
| 1229 | 1194G>A | Silent |

| | | | | |
|---|---|---|---|---|
| CBR | J04056 | 114830 | GEN-13O | Human carbonyl |

reductase mRNA, complete cds

| | | |
|---|---|---|
| 1060 | 967G>A | 3′ |

| | | | | |
|---|---|---|---|---|
| CBS | L00972 | 236200 | GEN-UV | Human cystathionine- |

beta synthase (CBS) mRNA

| | | |
|---|---|---|
| 1022 | 1022T>C | 3′ |
| 1403 | 1403T>C | 3′ |

| | | | | |
|---|---|---|---|---|
| CFTR | M28668 | 602421 | GEN-2DF | Human cystic fibrosis |

mRNA, encoding a presumed transmembrane conductance regulator
(CFTR)

| | | |
|---|---|---|
| 125 | (−8)G>C | 5′ |
| 156 | 24G>A | Silent |
| 223 | 91C>T | R31C |
| 263 | 131A>T | D44V |
| 345 | 213T>C | Silent |
| 356 | 224G>A | R75Q |
| 492 | 360G>A | Silent |
| 545 | 413T>C | L138P |
| 676 | 544A>G | S182G |
| 741 | 609C>T | Silent |
| 1047 | 915C>T | Silent |
| 1059 | 927C>G | Silent |
| 1096 | 964G>A | V322M |
| 1104 | 972C>G | Silent |
| 1184 | 1052C>G | T351S |
| 1191 | 1059A>C | Q353H |
| 1296 | 1164G>T | Silent |
| 1572 | 1440T>C | Silent |
| 1650 | 1518C>G | I506M |
| 1651 | 1519A>G | I507V |
| 1655 | 1523T>G | F508C |
| 1773 | 1641A>T | Silent |
| 1859 | 1727G>C | G576A |
| 2092 | 1960A>G | S654G |
| 2134 | 2002C>T | R668C |
| 2209 | 2077T>C | F693L |
| 2238 | 2106C>G | Silent |
| 2553 | 2421A>G | I807M |
| 2691 | 2559T>C | Silent |
| 2694 | 2562T>G | Silent |
| 2839 | 2707T>C | Y903H |
| 2858 | 2726G>T | S909I |
| 2901 | 2769C>T | Silent |
| 3212 | 3080T>C | I1027T |
| 3309 | 3177A>G | Silent |
| 3332 | 3200C>T | A1067V |
| 3333 | 3201C>T | Silent |
| 3336 | 3204C>T | Silent |
| 3384 | 3252A>G | Silent |
| 3417 | 3285A>T | Silent |
| 3471 | 3339T>C | Silent |
| 3690 | 3558A>G | Silent |
| 3726 | 3594G>T | Silent |
| 3791 | 3659C>T | T12201 |
| 3939 | 3807C>T | Silent |
| 4029 | 3897A>G | Silent |
| 4050 | 3918C>T | Silent |
| 4404 | 4272C>T | Silent |
| 4521 | 4389G>A | Silent |

| | | | | |
|---|---|---|---|---|
| CYP11B2 | D13752 | 124080 | GEN-CCD | Human CYP11B2 |

gene for steroid 18-hydroxylase, complete cds

| | | |
|---|---|---|
| 295 | 288T>C | Silent |
| 511 | 504C>T | Silent |
| 525 | 518A>G | K173R |
| 672 | 665A>C | N222T |
| 750 | 743T>C | I248T |
| 778 | 771T>G | F257L |
| 832 | 825C>T | Silent |
| 849 | 842A>G | N281S |
| 880 | 873G>A | Silent |
| 898 | 891G>A | Silent |
| 1023 | 1016T>C | I339T |
| 1155 | 1148A>T | E383V |
| 1164 | 1157T>C | V386A |
| 1177 | 1170G>A | Silent |
| 1310 | 1303G>A | G435S |
| 1322 | 1315C>T | H439Y |
| 1360 | 1353C>T | Silent |
| 1466 | 1459T>G | F487V |
| 1600 | 1593G>A | 3′ |

| | | | | |
|---|---|---|---|---|
| CYP1B1 | U03688 | 601771 | GEN-11Y | Human dioxin- |

inducible cytochrome P450 (CYP1B1) mRNA, complete cds

| | | |
|---|---|---|
| 1640 | 1294G>C | V432L |
| 1693 | 1347T>A | D449E |
| 1704 | 1358A>G | N453S |
| 2096 | 1750C>G | 3′ |
| 2316 | 1970T>A | 3′ |

| | | | | |
|---|---|---|---|---|
| CYP51 | U23942 | 601637 | GEN-27K | Human lanosterol 14- |

demethylase cytochrome P450 (CYP51) mRNA, complete cds

| | | |
|---|---|---|
| 2283 | 2161G>T | 3′ |

| | | | | |
|---|---|---|---|---|
| D12614 | D12614 | 153440 | GEN-QD | Human mRNA for |

lymphotoxin (TNF-beta), complete cds

| | | |
|---|---|---|
| 319 | 179C>A | T60N |

| | | | | |
|---|---|---|---|---|
| D13811 | D13811 | 238310 | GEN-AA | Glycine cleavage |

system: Protein T

| | | |
|---|---|---|
| 254 | 125A>G | H42R |
| 268 | 139G>A | G47R |
| 312 | 183delC | Frame |
| 935 | 806G>A | G269D |
| 955 | 826G>C | D276H |
| 1088 | 959G>A | R320H |

| | | | | |
|---|---|---|---|---|
| D17793 | D17793 | 603966 | GEN-20Q | Human mRNA for |

KIAA0119 gene, complete cds

| | | |
|---|---|---|
| 980 | 929G>C | S310T |

| | | | | |
|---|---|---|---|---|
| D26480 | D26480 | None | GEN-LBX | Human mRNA for |

leukotriene B4 omega-hydroxylase, complete cds

| | | |
|---|---|---|
| 2147 | 2106C>G | 3′ |

| | | | | |
|---|---|---|---|---|
| D49737 | D49737 | 602413 | GEN-2Z7 | Homo sapiens mRNA |

for cytochrome b large subunit of complex II, complete cds

| | | |
|---|---|---|
| 908 | 784G>A | 3′ |

| | | | | |
|---|---|---|---|---|
| D87030 | D87030 | None | GEN-4EZ | Serotonin receptor |

5HT-2A, 5′ upstream region

| | | |
|---|---|---|
| 178 | 178G>A | 3′ |

| | | | | |
|---|---|---|---|---|
| DDH1 | U05598 | 600450 | GEN-184 | Human dihydrodiol |

dehydrogenase mRNA, complete cds

| | | |
|---|---|---|
| 126 | 103C>T | Silent |
| 149 | 126A>T | Silent |
| 179 | 156A>T | Silent |
| 1020 | 997G>A | 3′ |

| | | | | |
|---|---|---|---|---|
| ESD | M13450 | 133280 | GEN-1O7 | Human esterase D |

mRNA, 3′ end

| | | |
|---|---|---|
| 614 | 614G>A | G205E |

| | | | | |
|---|---|---|---|---|
| FABP2 | M10050 | 134640 | GEN-1IE | Human liver fatty acid |

binding protein (FABP) mRNA, complete cds

| | | |
|---|---|---|
| 202 | 160G>A | A54T |

| | | | | |
|---|---|---|---|---|
| FACL1 | L09229 | 152425 | GEN-1GI | Human long-chain |

acyl-coenzyme A synthetase (FACL1) mRNA, complete cds

| | | |
|---|---|---|
| 3026 | 2953G>A | 3′ |

| | | | | |
|---|---|---|---|---|
| GC | M12654 | 139200 | GEN-1MN | Human serum vitamin |

D-binding protein (hDBP) mRNA, complete cds

| | | |
|---|---|---|
| 45 | 17T>C | V6A |

| | | | | |
|---|---|---|---|---|
| GPX1 | Y00433 | 138320 | GEN-TJ | Human mRNA for |

glutathione peroxidase (EC 1.11.1.9.)

| | | |
|---|---|---|
| 273 | (−46)C>T | 5′ |
| 911 | 593C>T | P198L |

| | | | | |
|---|---|---|---|---|
| GPX3 | X58295 | 138321 | GEN-38S | Human GPx-3 mRNA |

for plasma glutathione peroxidase

| | | |
|---|---|---|
| 1354 | 1306C>T | 3′ |

| | | | | |
|---|---|---|---|---|
| GPX4 | X71973 | 138322 | GEN-3L1 | H.sapiens GPx-4 |

mRNA for phospholipid hydroperoxide glutathione peroxidase

| | | |
|---|---|---|
| 738 | 658C>T | 3′ |

| | | | | |
|---|---|---|---|---|
| GSS | U34683 | 601002 | GEN-2LF | Human glutathione |

synthetase mRNA, complete cds

| | | |
|---|---|---|
| 1737 | 1697C>T | 3′ |

| | | | | |
|---|---|---|---|---|
| GSTM3 | J05459 | 138390 | GEN-17O | Human glutathione |

transferase M3 (GSTM3) mRNA, complete cds

| | | |
|---|---|---|
| 687 | 670G>A | V224I |

| | | | | |
|---|---|---|---|---|
| GSTP1 | X06547 | 134660 | GEN-19N | Human mRNA for |

class Pi glutathione S-transferase (GST-Pi; E.C.2.5.1.18)

| | | |
|---|---|---|
| 319 | 313A>G | I105V |
| 561 | 555C>T | Silent |

| | | | | |
|---|---|---|---|---|
| GSTT2 | L38503 | 600437 | GEN-2PC | Homo sapiens |

glutathione S-transferase theta 2 (GSTT2) mRNA, complete cds

| | | |
|---|---|---|
| 888 | 888G>T | 3′ |

| | | | | |
|---|---|---|---|---|
| GUSB | M15182 | 253220 | GEN-1TH | Endo-beta-D- |

glucuronidase

| | | |
|---|---|---|
| 698 | 672C>T | Silent |
| 1170 | 1144C>T | R382C |
| 1882 | 1856C>T | A619V |
| 1972 | 1946C>T | P649L |

| | | | | |
|---|---|---|---|---|
| HADHB | D16481 | 143450 | GEN-1Y5 | Human mRNA for |

mitochondrial 3-ketoacyl-CoA thiolase beta-subunit of trifunctional
protein, complete cds

| | | |
|---|---|---|
| 228 | 182G>A | R61H |
| 786 | 740G>A | R247H |
| 834 | 788A>G | D263G |

| | | | | |
|---|---|---|---|---|
| HLA- | X00532 | 142858 | GEN-U2 | Human mRNA for |
| DPB1 | | | | |

SB beta-chain (clone pII-beta-7)

| | | |
|---|---|---|
| 568 | 568A>G | R190G |

| | | | | |
|---|---|---|---|---|
| HTR1E | M91467 | 182132 | GEN-4EE | Serotonin 5-HT |

receptors 5-HT1E

| | | |
|---|---|---|
| 1097 | 531C>T | Silent |
| 1351 | 785C>T | S262F |

| | | | | |
|---|---|---|---|---|
| ID1 | X77956 | 600349 | GEN-3QL | H.sapiens Id1 mRNA |

| | | |
|---|---|---|
| 851 | 816G>A | 3′ |

| | | | | |
|---|---|---|---|---|
| J03037 | J03037 | 259730 | GEN-2I | Carbonic anhydrase II |

| | | |
|---|---|---|
| 627 | 562C>T | Silent |

| | | | | |
|---|---|---|---|---|
| J03143 | J03143 | 107470 | GEN-ZK | Human interferon- |

gamma receptor mRNA, complete cds

| | | |
|---|---|---|
| 395 | 347C>A | Frame |

| | | | | |
|---|---|---|---|---|
| J03490 | J03490 | 246900 | GEN-C5 | Dihydrolipoamide |

dehydrogenase (E3 component of pyruvate dehydrogenase complex,
2-oxo-glutarate complex, branched chain keto acid dehydrogenase
complex)

| | | |
|---|---|---|
| 1624 | 1548T>A | 3′ |
| 2088 | 2012T>C | 3′ |
| 2096 | 2020T>C | 3′ |
| 2142 | 2066G>T | 3′ |

| | | | | |
|---|---|---|---|---|
| J03571 | J03571 | 152390 | GEN-9 | Lipoxygenases: |

5-lipoxygenase (leukocytes)

| | | |
|---|---|---|
| 55 | 21C>T | Silent |
| 304 | 270G>A | Silent |
| 1762 | 1728A>T | Silent |

| | | | | |
|---|---|---|---|---|
| J03810 | J03810 | 138160 | GEN-C9 | Solute carrier family 2 |

(facilitated glucose transporter), member 2

| | | |
|---|---|---|
| 339 | 301G>A | V101I |
| 367 | 329C>T | T110I |
| 699 | 661C>T | Silent |
| 1475 | 1437C>T | Silent |
| 1544 | 1506G>A | Silent |

| | | | | |
|---|---|---|---|---|
| J03817 | J03817 | 138350 | GEN-9D | Glutathione S- |

transferase M1

| | | |
|---|---|---|
| 1008 | 993C>T | 3′ |

| | | | | |
|---|---|---|---|---|
| J04031 | J04031 | 172460 | GEN-CB | Methenyltetrahydro- |

folate cyclohydrolase

| | | |
|---|---|---|
| 931 | 878G>A | R293H |
| 3009 | 2956A>C | 3′ |

| | | | | |
|---|---|---|---|---|
| J05176 | J05176 | 107280 | GEN-PT | Human alpha-1- |

antichymotrypsin mRNA, 3′ end

| | | |
|---|---|---|
| 170 | 170T>C | L57P |

| | | | | |
|---|---|---|---|---|
| J05594 | J05594 | 601688 | GEN-E | Prostaglandin 15-OH |

dehydrogenase (PGDH)

| | | |
|---|---|---|
| 1448 | 1431G>A | 3′ |

| | | | | |
|---|---|---|---|---|
| K01171 | K01171 | 142860 | GEN-PB | Human HLA-DR |

alpha-chain mRNA

| | | |
|---|---|---|
| 416 | 402C>A | Silent |

| | | | | |
|---|---|---|---|---|
| K03191 | K03191 | 108330 | GEN-9E | Cytochrome P450, |

subfamily I (aromatic compound-inducible), polypeptide 1

| | | |
|---|---|---|
| 1470 | 1384G>A | V462I |
| 2220 | 2134T>C | 3′ |

| | | | | |
|---|---|---|---|---|
| K03195 | K03195 | 138140 | GEN-ZT | Human (HepG2) |

glucose transporter gene mRNA, complete cds

| | | |
|---|---|---|
| 943 | 764A>C | K255T |
| 2120 | 1941G>C | 3′ |

| | | | | |
|---|---|---|---|---|
| L02932 | L02932 | 170998 | GEN-KW4 | Human peroxisome |

proliferator activated receptor mRNA, complete cds

| | | |
|---|---|---|
| 896 | 680T>C | V227A |
| 978 | 762G>C | Silent |
| 1019 | 803T>C | V268A |
| 1442 | 1226G>C | R409T |
| 1651 | 1435C>T | 3′ |

| | | | | |
|---|---|---|---|---|
| L05597 | L05597 | 182134 | GEN-4EV | Serotonin 5-HT |

receptors 5-HT1F

| | | |
|---|---|---|
| 147 | (−78)C>T | 5′ |
| 752 | 528C>T | Silent |
| 1007 | 783T>A | Silent |
| 1330 | 1106A>T | 3′ |

| | | | | |
|---|---|---|---|---|
| L10819 | L10819 | 171150 | GEN-LVD | Homo sapiens aryl |

sulfotransferase mRNA, complete cds

| | | |
|---|---|---|
| 676 | 638A>G | H213R |
| 705 | 667A>G | M223V |

| | | | | |
|---|---|---|---|---|
| L11695 | L11695 | 190181 | GEN-MDJ | Human activin |

receptor-like kinase (ALK-5) mRNA, complete cds

| | | |
|---|---|---|
| 1644 | 1568A>G | 3′ |
| 1657 | 1581G>A | 3′ |

| | | | | |
|---|---|---|---|---|
| L11696 | L11696 | 104614 | GEN-D6 | Solute carrier family 3 |

(cystine, dibasic and neutral amino acid transporters, activator of cystine,
dibasic and neutral amino acid transport), member 1

| | | |
|---|---|---|
| 2232 | 2189T>C | 3′ |

| | | | | |
|---|---|---|---|---|
| L14754 | L14754 | 600502 | GEN-D9 | DNA-binding protein |

(SMBP2)

| | | |
|---|---|---|
| 244 | 195C>A | Silent |
| 244 | 195C>G | Silent |
| 390 | 341T>C | V114A |
| 390 | 341T>G | V114G |

| | | | | |
|---|---|---|---|---|
| L19067 | L19067 | 164014 | GEN-DE | TRANSCRIPTION |

FACTOR P65

| | | |
|---|---|---|
| 2024 | 1986C>T | 3′ |
| 2310 | 2272G>T | 3′ |

| | | | | |
|---|---|---|---|---|
| L24470 | L24470 | 600563 | GEN-O | PROSTAGLANDIN F |

RECEPTOR

| | | |
|---|---|---|
| 2203 | 1966A>C | 3′ |
| 2299 | 2062A>G | 3′ |

| | | | | |
|---|---|---|---|---|
| L38928 | L38928 | 604197 | GEN-2PT | Homo sapiens 5,10- |

methenyltetrahydrofolate synthetase mRNA, complete cds

| | | |
|---|---|---|
| 617 | 604A>G | T202A |

| | | | | |
|---|---|---|---|---|
| L40904 | L40904 | 601487 | GEN-2SK | H. sapiens peroxisome |

proliferator activated receptor gamma, complete cds

| | | |
|---|---|---|
| 206 | 34C>G | P12A |
| 426 | 254C>A | P85Q |

| | | | | |
|---|---|---|---|---|
| L42812 | L42812 | 100740 | GEN-LUN | Homo sapiens |

acetylcholinesterase (ACHE) gene, exons 2-6

| | | |
|---|---|---|
| 1092 | 1092T>A | Genomic |
| 1151 | 1151C>A | Genomic |
| 1871 | 1871C>T | Genomic |
| 3290 | 3290C>G | Genomic |

| | | | | |
|---|---|---|---|---|
| L48513 | L48513 | 602447 | GEN-2YD | Homo sapiens |

paraoxonase 2 (PON2) mRNA, complete cds

| | | |
|---|---|---|
| 460 | 443C>G | A148G |
| 949 | 932G>C | C311S |

| | | | | |
|---|---|---|---|---|
| L78207 | L78207 | 600509 | GEN-5Q | Cell surface receptor |

for sulfonylureas on pancreatic b cells

| | | |
|---|---|---|
| 245 | 207T>C | Silent |
| 2315 | 2277C>T | Silent |
| 3857 | 3819G>A | Silent |

| | | | | |
|---|---|---|---|---|
| LIG1 | M36067 | 126391 | GEN-2MS | Human DNA ligase I |

mRNA, complete cds

| | | |
|---|---|---|
| 630 | 510A>C | Silent |

| | | | | |
|---|---|---|---|---|
| LIPC | J03540 | 151670 | GEN-11J | Human hepatic lipase |

mRNA, complete cds

| | | |
|---|---|---|
| 648 | 644G>A | S215N |
| 676 | 672C>G | Silent |
| 1323 | 1319G>A | S440N |
| 1441 | 1437C>A | Silent |

| | | | | |
|---|---|---|---|---|
| M10901 | M10901 | 138040 | GEN-2W | Corticosteroid nuclear |

receptor b

| | | |
|---|---|---|
| 198 | 66G>A | Silent |
| 200 | 68G>A | R23K |
| 325 | 193T>G | F65V |
| 936 | 804C>T | Silent |
| 1220 | 1088A>G | N363S |
| 1226 | 1094A>G | N365S |
| 2024 | 1892-1893delAG | Frame |
| 2054 | 1922A>T | D641V |
| 2372 | 2240T>G | I747S |
| 2391 | 2259A>C | L753F |
| 2430 | 2298T>C | Silent |
| 3691 | 3559T>C | 3′ |
| 4172 | 4040G>T | 3′ |
| 4654 | 4522A>G | 3′ |

| | | | | |
|---|---|---|---|---|
| M11050 | M11050 | 138040 | GEN-7Y | Glucocorticoid |

receptor

| | | |
|---|---|---|
| 198 | 66G>A | Silent |
| 200 | 68G>A | R23K |
| 325 | 193T>G | F65V |
| 936 | 804C>T | Silent |
| 1220 | 1088A>G | N363S |
| 1226 | 1094A>G | N365S |
| 3134 | 3002G>T | 3′ |
| 3669 | 3537A>G | 3′ |

| | | | | |
|---|---|---|---|---|
| M12959 | M12959 | 186880 | GEN-S | CD3 glycoprotein on T |

lymphocytes

| | | |
|---|---|---|
| 1249 | 1113C>G | 3′ |
| 1343 | 1207T>C | 3′ |
| 1345 | 1209G>C | 3′ |
| 1394 | 1258T>G | 3′ |
| 1463 | 1327G>A | 3′ |

| | | | | |
|---|---|---|---|---|
| M14565 | M14565 | 118485 | GEN-30 | “Cytochrome P450, |

subfamily XIA (cholesterol side chain cleavage)”

| | | |
|---|---|---|
| 984 | 940G>A | E314K |

| | | | | |
|---|---|---|---|---|
| M14758 | M14758 | 171050 | GEN-1S6 | P glycoprotein 1 |

| | | |
|---|---|---|
| 978 | 554T>G | V185G |
| 979 | 555T>A | Silent |
| 4460 | 4036A>G | 3′ |

| | | | | |
|---|---|---|---|---|
| M15856 | M15856 | 238600 | GEN-33 | Lipoprotein lipase |

| | | |
|---|---|---|
| 136 | (−39)T>C | 5′ |
| 280 | 106G>A | D36N |
| 438 | 264T>A | Frame |
| 447 | 273G>A | Frame |
| 474 | 300C>A | Frame |
| 480 | 306A>C | R102S |
| 511 | 337T>C | W113R |
| 571 | 397C>T | Frame |
| 680 | 506G>A | G169E |
| 722 | 548A>G | D183G |
| 770 | 596C>G | S199C |
| 781 | 607G>A | A203T |
| 795 | 621C>G | D207E |
| 818 | 644G>A | G215E |
| 836 | 662T>C | I221T |
| 839 | 665G>A | G222E |
| 867 | 693C>G | D231E |
| 875 | 701C>T | P234L |
| 916 | 742delG | Frame |
| 983 | 809G>A | R270H |
| 985 | 811T>A | S271T |
| 1003 | 829G>A | D277N |
| 1036 | 862G>A | A288T |
| 1127 | 953A>G | N318S |
| 1255 | 1081G>A | A361T |
| 1282 | 1108G>A | V370M |
| 1348 | 1174C>G | L392V |
| 1401 | 1227G>A | Frame |
| 1453 | 1279G>A | A427T |
| 1508 | 1334G>A | C445Y |
| 1595 | 1421C>G | Frame |
| 1973 | 1799T>C | 3′ |

| | | | | |
|---|---|---|---|---|
| M15872 | M15872 | 138360 | GEN-QS | Human glutathione S- |

transferase 2 (GST) mRNA, complete cds

| | | |
|---|---|---|
| 170 | 115G>T | Frame |

| | | | | |
|---|---|---|---|---|
| M16541 | M16541 | 177400 | GEN-35 | Butyrylcholinesterase |

| | | |
|---|---|---|
| 422 | 293A>G | D98G |
| 557 | 428G>A | G143D |
| 568 | 439C>T | Frame |
| 596 | 467A>G | Y156C |
| 961 | 832A>C | T278P |
| 1201 | 1072T>A | L358I |
| 1306 | 1177G>A | G393R |
| 1382 | 1253G>T | G418V |
| 1549 | 1420T>G | F474V |
| 1564 | 1435G>T | Frame |
| 1703 | 1574A>T | E525V |
| 1756 | 1627C>T | R543C |
| 1828 | 1699G>A | A567T |
| 2127 | 1998A>G | 3′ |

| | | | | |
|---|---|---|---|---|
| M16827 | M16827 | 201450 | GEN-EI | Acyl-Coenzyme A |

dehydrogenase, C-4 to C-12 straight chain

| | | |
|---|---|---|
| 1179 | 1161A>G | Silent |
| 1956 | 1938T>C | 3′ |

| | | | | |
|---|---|---|---|---|
| M19154 | M19154 | 190220 | GEN-R0 | Human transforming |

growth factor-beta-2 mRNA, complete cds

| | | |
|---|---|---|
| 1990 | 1523C>A | 3′ |
| 1990 | 1523C>T | 3′ |
| 1997 | 1530A>T | 3′ |

| | | | | |
|---|---|---|---|---|
| M21054 | M21054 | 172410 | GEN-3B | Phospholipase A-2 |

(PLA-2) lung

| | | |
|---|---|---|
| 160 | 123C>T | Silent |
| 259 | 222T>C | Silent |
| 303 | 266A>C | N89T |
| 304 | 267C>A | N89K |
| 331 | 294G>A | Silent |

| | | | | |
|---|---|---|---|---|
| M22324 | M22324 | 151530 | GEN-25R | Human amino- |

peptidase N/CD13 mRNA encoding aminopeptidase N, complete cds

| | | |
|---|---|---|
| 3053 | 2933G>C | 3′ |

| | | | | |
|---|---|---|---|---|
| M26393 | M26393 | 201470 | GEN-EW | Acyl-Coenzyme A |

dehydrogenase, C-2 to C-3 short chain

| | | |
|---|---|---|
| 353 | 321T>C | Silent |
| 543 | 511C>T | R171W |
| 1022 | 990C>T | Silent |
| 1292 | 1260G>C | 3′ |
| 1797 | 1765A>G | 3′ |

| | | | | |
|---|---|---|---|---|
| M27819 | M27819 | 109270 | GEN-EY | “Solute carrier family |

4, anion exchanger, member 1 (MEDIATES EXCHANGE OF
INORGANIC ANIONS ACROSS THE MEMBRANE)”

| | | |
|---|---|---|
| 1038 | 924G>A | Silent |
| 1353 | 1239C>T | Silent |
| 1363 | 1249C>T | Silent |
| 1437 | 1323G>A | Silent |
| 1438 | 1324A>T | I442F |
| 1870 | 1756A>T | M586L |
| 2067 | 1953C>T | Silent |
| 2214 | 2100C>T | Silent |
| 2698 | 2584G>A | V862I |
| 3119 | 3005G>A | 3′ |
| 3158 | 3044C>A | 3′ |

| | | | | |
|---|---|---|---|---|
| M29474 | M29474 | 179615 | GEN-MIU | Human recombination |

activating protein (RAG-1) gene, complete cds

| | | |
|---|---|---|
| 579 | 467C>T | A156V |

| | | | | |
|---|---|---|---|---|
| M29882 | M29882 | 107670 | GEN-6R | Apolipoprotein A-II |

| | | |
|---|---|---|
| 256 | 247C>T | Silent |

| | | | | |
|---|---|---|---|---|
| M30938 | M30938 | 194364 | GEN-F5 | ATP-DEPENDENT |

DNA HELICASE II, 86 KD SUBUNIT

| | | |
|---|---|---|
| 3150 | 3123T>A | 3′ |

| | | | | |
|---|---|---|---|---|
| M31523 | M31523 | 147141 | GEN-F7 | Transcription factor 3 |

(E2A immunoglobulin enhancer binding factors E12/E47)

| | | |
|---|---|---|
| 1332 | 1302G>A | Silent |

| | | | | |
|---|---|---|---|---|
| M33195 | M33195 | 147139 | GEN-2JR | Human Fc-epsilon- |

receptor gamma-chain mRNA, complete cds

| | | |
|---|---|---|
| 446 | 421T>G | 3′ |

| | | | | |
|---|---|---|---|---|
| M34479 | M34479 | 179060 | GEN-F9 | Pyruvate |

dehydrogenase (lipoamide) beta

| | | |
|---|---|---|
| 1323 | 1323C>A | 3′ |

| | | | | |
|---|---|---|---|---|
| M55040 | M55040 | 100740 | GEN-3Q | acetylcholinesterase |

| | | |
|---|---|---|
| 1154 | 998T>A | V333E |
| 1213 | 1057C>A | H353N |
| 1587 | 1431C>T | Silent |

| | | | | |
|---|---|---|---|---|
| M55531 | M55531 | 138230 | GEN-FF | Solute carrier family 2 |

(facilitated glucose transporter), member 5

| | | |
|---|---|---|
| 51 | (−25)G>A | 5′ |

| | | | | |
|---|---|---|---|---|
| M57899 | M57899 | 191740 | GEN-38A | Human bilirubin UDP- |

glucuronosyltransferase isozyme 1 mRNA, complete cds

| | | |
|---|---|---|
| 2057 | 2042C>G | 3′ |

| | | | | |
|---|---|---|---|---|
| M58525 | M58525 | 116790 | GEN-3S | Catechol-o- |

methyltransferase

| | | |
|---|---|---|
| 390 | 186T>C | Silent |
| 418 | 214G>T | A72S |
| 612 | 408C>G | Silent |
| 676 | 472A>G | M158V |

| | | | | |
|---|---|---|---|---|
| M58664 | M58664 | 103000 | GEN-395 | Homo sapiens CD24 |

signal transducer mRNA, complete cds

| | | |
|---|---|---|
| 226 | 170C>T | A57V |

| | | | | |
|---|---|---|---|---|
| M59941 | M59941 | 138981 | GEN-62 | “Granulocyte- |

macrophage (Colony stimulating factor 2 receptor, beta, low-affinity)”

| | | |
|---|---|---|
| 730 | 702C>T | Silent |
| 773 | 745G>C | E249Q |
| 1306 | 1278C>T | Silent |
| 1835 | 1807C>A | P603T |
| 1968 | 1940G>T | G647V |
| 1972 | 1944G>A | Silent |
| 1982 | 1954G>A | V652M |
| 2428 | 2400G>A | Silent |

| | | | | |
|---|---|---|---|---|
| M59979 | M59979 | 176805 | GEN-Z | Cyclooxygenase 1 |

COX1

| | | |
|---|---|---|
| 328 | 323G>A | R108Q |
| 644 | 639C>A | Silent |
| 714 | 709C>A | L237M |
| 956 | 951T>C | Silent |
| 1081 | 1076A>G | K359R |
| 1332 | 1327A>G | I443V |
| 1404 | 1399A>G | K467E |
| 1446 | 1441G>A | V481I |
| 1924 | 1919T>C | 3′ |
| 1966 | 1961T>C | 3′ |
| 2055 | 2050T>G | 3′ |

| | | | | |
|---|---|---|---|---|
| M60761 | M60761 | 156569 | GEN-FL | O-6-methylguanine- |

DNA methyltransferase

| | | |
|---|---|---|
| 174 | 159C>T | Silent |
| 210 | 195G>C | W65C |
| 265 | 250C>T | L84F |
| 442 | 427A>G | I143V |
| 493 | 478G>A | G160R |
| 548 | 533A>G | K178R |
| 582 | 567G>A | Silent |

| | | | | |
|---|---|---|---|---|
| M61855 | M61855 | 601130 | GEN-3C1 | Human cytochrome |

P4502C9 (CYP2C9) mRNA, clone 25

| | | |
|---|---|---|
| 442 | 442C>T | 3′ |
| 1087 | 1087C>A | 3′ |

| | | | | |
|---|---|---|---|---|
| M63012 | M63012 | 168820 | GEN-9F | Paraoxonase 1 |

| | | |
|---|---|---|
| 584 | 575A>G | Q192R |

| | | | | |
|---|---|---|---|---|
| M64799 | M64799 | 162020 | GEN-4DN | Histamine receptor H2 |

| | | |
|---|---|---|
| 398 | 398T>C | V133A |
| 525 | 525A>T | K175N |
| 620 | 620A>G | K207R |
| 649 | 649A>G | N217D |
| 692 | 692A>G | K231R |
| 802 | 802G>A | V268M |

| | | | | |
|---|---|---|---|---|
| M65105 | M65105 | 163970 | GEN-14 | Norepinephrine |

transporter

| | | |
|---|---|---|
| 897 | 837C>G | Silent |
| 935 | 875A>C | N292T |
| 1126 | 1066G>C | V356L |
| 1165 | 1105G>C | A369P |
| 1347 | 1287G>A | Silent |
| 1429 | 1369G>C | A457P |
| 1702 | 1642T>C | Y548H |

| | | | | |
|---|---|---|---|---|
| M69043 | M69043 | 164008 | GEN-3IZ | Homo sapiens MAD-3 |

mRNA encoding IkB-like activity, complete cds

| | | |
|---|---|---|
| 1050 | 956T>C | 3′ |
| 1174 | 1080A>G | 3′ |

| | | | | |
|---|---|---|---|---|
| M69177 | M69177 | 309860 | GEN-3Y | Monoamine oxidase B |

| | | |
|---|---|---|
| 1538 | 1461C>T | Silent |

| | | | | |
|---|---|---|---|---|
| M69226 | M69226 | 309850 | GEN-3Z | Monoamine oxidase A |

| | | |
|---|---|---|
| 435 | 385A>C | Silent |
| 936 | 886C>T | Frame |
| 941 | 891T>G | Silent |
| 1076 | 1026A>T | Silent |
| 1460 | 1410C>T | Silent |
| 1609 | 1559A>G | K520R |

| | | | | |
|---|---|---|---|---|
| M74096 | M74096 | 201460 | GEN-G0 | Acyl-Coenzyme A |

dehydrogenase, long chain

| | | |
|---|---|---|
| 913 | 908G>C | S303T |

| | | | | |
|---|---|---|---|---|
| M76180 | M76180 | 107930 | GEN-16 | L-aromatic amino acid |

decarboxylase

| | | |
|---|---|---|
| 118 | 49G>A | V17M |
| 698 | 629C>T | P210L |
| 718 | 649A>G | M217V |

| | | | | |
|---|---|---|---|---|
| M80646 | M80646 | 274180 | GEN-40 | Thromboxane synthase |

| | | |
|---|---|---|
| 654 | 483C>A | D161E |
| 658 | 487C>A | L163I |
| 943 | 772A>G | K258E |
| 952 | 781A>G | R261G |
| 1120 | 949C>A | Q317K |
| 1166 | 995T>C | I332T |
| 1240 | 1069C>G | L357V |
| 1340 | 1169G>T | G390V |
| 1444 | 1273C>T | R425C |
| 1459 | 1288G>A | A430T |
| 1476 | 1305C>T | Silent |

| | | | | |
|---|---|---|---|---|
| M81590 | M81590 | 182131 | GEN-3VZ | Serotonin receptor |

5HT-1B, cDNA

| | | |
|---|---|---|
| 190 | 129C>T | Silent |
| 922 | 861G>C | Silent |

| | | | | |
|---|---|---|---|---|
| M81757 | M81757 | 603474 | GEN-3W6 | H.sapiens S19 |

ribosomal protein mRNA, complete cds

| | | |
|---|---|---|
| 1 | (−22)C>A | 5′ |
| 45 | 23A>C | D8A |
| 45 | 23A>T | D8V |

| | | | | |
|---|---|---|---|---|
| M81768 | M81768 | 107310 | GEN-G6 | Solute carrier family 9 |

(sodium/hydrogen exchanger)

| | | |
|---|---|---|
| 3080 | 3027T>C | 3′ |

| | | | | |
|---|---|---|---|---|
| M90100 | M90100 | 600262 | GEN-1A | Cyclooxygenase 2 |

COX2

| | | |
|---|---|---|
| 1306 | 1209T>C | Silent |
| 1560 | 1463A>G | E488G |
| 1629 | 1532T>C | V511A |
| 1852 | 1755C>A | Silent |
| 2409 | 2312G>A | 3′ |

| | | | | |
|---|---|---|---|---|
| M94859 | M94859 | 114217 | GEN-GP | Calnexin |

| | | |
|---|---|---|
| 3011 | 2916G>T | 3′ |

| | | | | |
|---|---|---|---|---|
| MBL | X15422 | 154545 | GEN-1U2 | Human mRNA for |

mannose-binding protein C

| | | |
|---|---|---|
| 226 | 161G>A | G54D |
| 235 | 170G>A | G57E |

| | | | | |
|---|---|---|---|---|
| MDCR | L13385 | 601545 | GEN-1O6 | Homo sapiens (clone |

71) Miller-Dieker lissencephaly protein (LIS1) mRNA, complete cds

| | | |
|---|---|---|
| 239 | 22C>T | Frame |
| 275 | 58C>T | R20C |
| 663 | 446A>G | H149R |
| 716 | 499T>C | S167P |
| 1034 | 817C>T | Frame |
| 5175 | 4958C>A | 3′ |

| | | | | |
|---|---|---|---|---|
| MHC2TA | X74301 | 600005 | GEN-3N5 | H.sapiens mRNA for |

MHC class II transactivator

| | | |
|---|---|---|
| 1614 | 1499C>G | A500G |

| | | | | |
|---|---|---|---|---|
| NMOR1 | J03934 | 125860 | GEN-12L | Human, NAD(P)H: |

menadione oxidoreductase mRNA, complete cds

| | | |
|---|---|---|
| 609 | 559C>T | P187S |

| | | | | |
|---|---|---|---|---|
| PDHA1 | X52709 | 312170 | GEN-33Y | Human mRNA for |

brain pyruvate dehydrogenase (EC 1.2.4.1)

| | | |
|---|---|---|
| 1337 | 1283C>T | 3′ |

| | | | | |
|---|---|---|---|---|
| PLA2G2A | M22430 | 172411 | GEN-25V | Human RASF-A |

PLA2 mRNA, complete cds

| | | |
|---|---|---|
| 231 | 96G>C | Silent |
| 267 | 132C>T | Silent |
| 278 | 143-144delGT | Frame |
| 643 | 508C>T | 3′ |
| 700 | 565G>C | 3′ |

| | | | | |
|---|---|---|---|---|
| PNMT | J03727 | 171190 | GEN-120 | Human |

phenylethanolamine N-methyltransferase mRNA, complete cds

| | | |
|---|---|---|
| 462 | 456A>G | Silent |
| 568 | 562A>T | S188C |
| 638 | 632T>A | L211H |
| 656 | 650T>A | L217Q |
| 767 | 761G>A | R254H |
| 832 | 826T>A | W276R |

| | | | | |
|---|---|---|---|---|
| PTAFR | M76674 | 173393 | GEN-3P9 | Platelet-activating |

factor receptor

| | | |
|---|---|---|
| 696 | 671C>A | A224D |

| | | | | |
|---|---|---|---|---|
| S77127 | S77127 | None | GEN-MTU | Superoxide dismutase |

2 (manganese), promoter and genomic

| | | |
|---|---|---|
| 1183 | 1183C>T | Genomic |
| 1735 | 1735T>C | Genomic |
| 4903 | 4903G>A | Genomic |
| 7939 | 7939G>A | Genomic |

| | | | | |
|---|---|---|---|---|
| SLC12A3 | U44128 | 600968 | GEN-CCX | Human thiazide- |

sensitive Na-Cl cotransporter (hTSC) mRNA, complete cds

| | | |
|---|---|---|
| 1884 | 1884G>A | Silent |
| 2142 | 2142C>T | Silent |
| 2625 | 2625C>T | Silent |

| | | | | |
|---|---|---|---|---|
| SLC2A4 | M20747 | 138190 | GEN-23Q | Human insulin- |

responsive glucose transporter (GLUT4) mRNA, complete cds

| | | |
|---|---|---|
| 378 | 233C>G | T78S |
| 535 | 390C>T | Silent |

| | | | | |
|---|---|---|---|---|
| SLC6A1 | X54673 | 137165 | GEN-358 | H.sapiens GAT1 |

mRNA for GABA transporter

| | | |
|---|---|---|
| 240 | 6G>A | Silent |
| 885 | 651G>T | Silent |

| | | | | |
|---|---|---|---|---|
| SLC6A3 | L24178 | 126455 | GEN-283 | Homo sapiens |

dopamine transporter mRNA, complete cds

| | | |
|---|---|---|
| 133 | 114C>T | Silent |
| 169 | 150G>T | Silent |
| 181 | 162C>T | Silent |
| 729 | 710G>A | R237Q |
| 1234 | 1215G>A | Silent |
| 1750 | 1731C>T | Silent |

| | | | | |
|---|---|---|---|---|
| SOD3 | J02947 | 185490 | GEN-Y3 | Human extracellular- |

superoxide dismutase (SOD3) mRNA, complete cds

| | | |
|---|---|---|
| 760 | 691C>G | R231G |

| | | | | |
|---|---|---|---|---|
| TAP2 | Z22935 | 170261 | GEN-26P | H.sapiens TAP2B |

mRNA, complete CDS

| | | |
|---|---|---|
| 1690 | 1662G>A | Silent |
| 1746 | 1718A>G | D573G |
| 2021 | 1993G>A | A665T |

| | | | | |
|---|---|---|---|---|
| TCN2 | M60396 | 275350 | GEN-3AX | Human transcobalamin |

II (TCII) mRNA, complete cds

| | | |
|---|---|---|
| 813 | 776C>G | P259R |
| 1623 | 1586C>A | 3′ |
| 1635 | 1598C>A | 3′ |

| | | | | |
|---|---|---|---|---|
| TGFBR3 | L07594 | 600742 | GEN-1EA | Human transforming |

growth factor-beta type III receptor (TGF-beta) mRNA, complete cds

| | | |
|---|---|---|
| 150 | (−199)G>A | 5′ |
| 150 | (−199)G>C | 5′ |
| 3957 | 3609A>C | 3′ |
| 3966 | 3618G>C | 3′ |

| | | | | |
|---|---|---|---|---|
| TPMT | U12387 | 187680 | GEN-1LY | Human thiopurine |

methyltransferase (TPMT) mRNA, complete cds

| | | |
|---|---|---|
| 314 | 238G>C | A80P |
| 536 | 460G>A | A154T |
| 720 | 644G>A | R215H |
| 795 | 719A>G | Y240C |

| | | | | |
|---|---|---|---|---|
| TRP2 | M55169 | 190470 | GEN-35U | Homo sapiens |

tripeptidyl peptidase II mRNA, 3′ end

| | | |
|---|---|---|
| 3637 | 3637G>A | 3′ |

| | | | | |
|---|---|---|---|---|
| U00672 | U00672 | 146933 | GEN-4A | Interleukin 10 receptor |

| | | |
|---|---|---|
| 3524 | 3463A>G | 3′ |

| | | | | |
|---|---|---|---|---|
| U04636 | U04636 | 600262 | GEN-MVG | Cyclooxygenase 2, |

genomic sequence (not including promoter)

| | | |
|---|---|---|
| 671 | 671C>G | Genomic |
| 841 | 841T>G | Genomic |
| 2191 | 2191C>G | Genomic |
| 4719 | 4719T>C | Genomic |
| 5310 | 5310T>C | Genomic |
| 6551 | 6551A>G | Genomic |
| 6620 | 6620T>C | Genomic |
| 6843 | 6843C>A | Genomic |
| 7330 | 7330T>C | Genomic |
| 7401 | 7401G>A | Genomic |

| | | | | |
|---|---|---|---|---|
| U06088 | U06088 | 253000 | GEN-MP3 | Human N- |

acetylgalactosamine 6-sulphatase (GALNS) gene

| | | |
|---|---|---|
| 708 | 708T>C | Silent |

| | | | | |
|---|---|---|---|---|
| U08092 | U08092 | None | GEN-4C | Histamine |

N-methyltransferase

| | | |
|---|---|---|
| 353 | 314T>C | I105T |
| 978 | 939G>A | 3′ |

| | | | | |
|---|---|---|---|---|
| U09178 | U09178 | 274270 | GEN-HA | Dihydropyrimidine |

Dehydrogenase

| | | |
|---|---|---|
| 143 | 62G>A | R21Q |
| 166 | 85T>C | C29R |
| 784 | 703C>T | R235W |
| 1084 | 1003G>C | V335L |
| 1237 | 1156G>T | Frame |
| 1682 | 1601G>A | S534N |
| 1708 | 1627A>G | I543V |
| 2275 | 2194G>A | V7321I |
| 2738 | 2657G>A | R886H |
| 3002 | 2921A>T | D974V |
| 3064 | 2983G>T | V995F |

| | | | | |
|---|---|---|---|---|
| U09806 | U09806 | 236250 | GEN-4FZ | Human |

methylenetetrahydrofolate reductase mRNA, partial cds

| | | |
|---|---|---|
| 120 | 120T>C | Silent |
| 473 | 473G>A | R158Q |
| 550 | 550C>T | Frame |
| 668 | 668C>T | A223V |
| 1059 | 1059T>C | Silent |
| 1289 | 1289C>A | 3′ |
| 1308 | 1308T>C | 3′ |

| | | | | |
|---|---|---|---|---|
| U10417 | U10417 | 601295 | GEN-1IX | Homo sapiens ileal |

sodium-dependent bile acid transporter (SLC10-A2) mRNA, complete cds

| | | |
|---|---|---|
| 1109 | 511G>T | A171S |
| 1326 | 728T>C | L243P |
| 1383 | 785C>T | T262M |

| | | | | |
|---|---|---|---|---|
| U14510 | U14510 | 602698 | GEN-1RD | Human transcription |

factor NFATx mRNA, complete cds

| | | |
|---|---|---|
| 3564 | 3540A>C | 3′ |

| | | | | |
|---|---|---|---|---|
| U14650 | U14650 | 602243 | GEN-1RL | Human platelet- |

endothelial tetraspan antigen 3 mRNA, complete cds

| | | |
|---|---|---|
| 1263 | 1204G>C | 3′ |

| | | | | |
|---|---|---|---|---|
| U16660 | U16660 | 600696 | GEN-1YD | Human peroxisomal |

enoyl-CoA hydratase-like protein (HPXEL) mRNA, complete cds

| | | |
|---|---|---|
| 149 | 122A>C | E41A |

| | | | | |
|---|---|---|---|---|
| U19487 | U19487 | 176804 | GEN-4I | “PROSTAGLANDIN |

E2 RECEPTOR, EP2 SUBTYPE”

| | | |
|---|---|---|
| 1442 | 1286A>G | 3′ |

| | | | | |
|---|---|---|---|---|
| U36601 | U36601 | 603268 | GEN-IR | Heparan N- |

deacetylase/N-sulfotransferase-2

| | | |
|---|---|---|
| 2727 | 2700T>G | 3′ |
| 2972 | 2945A>G | 3′ |

| | | | | |
|---|---|---|---|---|
| U37519 | U37519 | 601917 | GEN-2OF | Human aldehyde |

dehydrogenase (ALDH8) mRNA, complete cds

| | | |
|---|---|---|
| 1871 | 1255C>T | 3′ |

| | | | | |
|---|---|---|---|---|
| U49516 | U49516 | 312861 | GEN-1Q | Serotonin 5-HT |

receptors 5-HT2C

| | | |
|---|---|---|
| 796 | 68G>C | C23S |
| 2831 | 2103T>G | 3′ |

| | | | | |
|---|---|---|---|---|
| U50040 | U50040 | 601582 | GEN-2ZR | Human signaling |

inositol polyphosphate 5 phosphatase SIP-110 mRNA, complete cds

| | | |
|---|---|---|
| 2882 | 2866C>T | H956Y |

| | | | | |
|---|---|---|---|---|
| U68162 | U68162 | 159530 | GEN-MJM | Human thrombopoietin |

receptor (MPL) gene

| | | |
|---|---|---|
| 218 | 152C>T | A51V |
| 547 | 481G>A | E161K |

| | | | | |
|---|---|---|---|---|
| U73338 | U73338 | 156570 | GEN-69 | Methionine Synthase |

| | | |
|---|---|---|
| 6750 | 6356G>A | 3′ |

| | | | | |
|---|---|---|---|---|
| U83411 | U83411 | 603105 | GEN-3Y1 | Homo sapiens |

carboxypeptidase Z precursor, mRNA, complete cds

| | | |
|---|---|---|
| 1788 | 1749G>A | Silent |

| | | | | |
|---|---|---|---|---|
| X02612 | X02612 | None | GEN-MW2 | Cytochrome P450 |

CYP1A1, promoter and genomic

| | | |
|---|---|---|
| 6819 | 6819G>A | Genomic |
| 7569 | 7569T>C | Genomic |

| | | | | |
|---|---|---|---|---|
| X02812 | X02812 | 190180 | GEN-XR | Human mRNA for |

transforming growth factor-beta (TGF-beta)

| | | |
|---|---|---|
| 870 | 29C>T | P10L |
| 915 | 74C>G | P25R |
| 1632 | 791C>T | T264I |

| | | | | |
|---|---|---|---|---|
| X02920 | X02920 | 107400 | GEN-PH | Human mRNA for |

alpha 1-antitrypsin carboxyterminal region (aa 268-394)

| | | |
|---|---|---|
| 195 | 195C>T | Silent |
| 327 | 327A>C | E109D |

| | | | | |
|---|---|---|---|---|
| X03348 | X03348 | 138040 | GEN-PL | Human mRNA for |

beta-glucocorticoid receptor (clone OB10)

| | | |
|---|---|---|
| 198 | 66G>A | Silent |
| 200 | 68G>A | R23K |
| 325 | 193T>G | F65V |
| 936 | 804C>T | Silent |
| 1220 | 1088A>G | N363S |
| 1226 | 1094A>G | N365S |
| 3134 | 3002G>T | 3′ |
| 3669 | 3537A>G | 3′ |

| | | | | |
|---|---|---|---|---|
| X03438 | X03438 | 138970 | GEN-PM | Human mRNA for |

granulocyte colony-stimulating factor (G-CSF)

| | | |
|---|---|---|
| 1180 | 1149C>T | 3′ |

| | | | | |
|---|---|---|---|---|
| X03663 | X03663 | 164770 | GEN-51 | Colony stimulating |

factor 1 receptor

| | | |
|---|---|---|
| 3206 | 2906A>G | Y969C |
| 3807 | 3507G>C | 3′ |

| | | | | |
|---|---|---|---|---|
| X03747 | X03747 | 182330 | GEN-KR | ATPase, Na+/K+ |

transporting, beta 1 polypeptide

| | | |
|---|---|---|
| 1773 | 1647C>T | 3′ |

| | | | | |
|---|---|---|---|---|
| X08006 | X08006 | 124030 | GEN-1FE | Human mRNA for |

cytochrome P450 db1

| | | |
|---|---|---|
| 100 | 100C>T | P34S |
| 124 | 124G>A | G42R |
| 137 | 137ˆ 138insT | Frame |
| 271 | 271C>A | L91M |
| 281 | 281A>G | H94R |
| 294 | 294C>G | Silent |
| 336 | 336C>T | Silent |
| 408 | 408G>C | Silent |
| 454 | 454delT | Frame |
| 505 | 505G>T | Frame |
| 635 | 635G>A | G212E |
| 775 | 775delA | Frame |
| 839 | 839-841delAGA | K281del |
| 840 | 840-842delGAA | K281del |
| 886 | 886C>T | R296C |
| 971 | 971A>C | H324P |
| 1203 | 1203G>A | Silent |
| 1262 | 1262T>C | L421P |
| 1457 | 1457G>C | S486T |

| | | | | |
|---|---|---|---|---|
| X13561 | X13561 | 147910 | GEN-1OH | Human mRNA for |

preprokallikrein (EC 3.4.21)

| | | |
|---|---|---|
| 469 | 433G>C | E145Q |
| 592 | 556A>G | K186E |
| 614 | 578T>A | V193E |

| | | | | |
|---|---|---|---|---|
| X13589 | X13589 | 107910 | GEN-56 | Aromatase (CYP19), |

cDNA

| | | |
|---|---|---|
| 914 | 790C>T | R264C |
| 1218 | 1094G>A | R365Q |
| 1247 | 1123C>T | R375C |
| 1347 | 1223delC | Frame |
| 1427 | 1303C>T | R435C |
| 1434 | 1310G>A | C437Y |

| | | | | |
|---|---|---|---|---|
| X13930 | X13930 | 122720 | GEN-1Q3 | Human CYP2A4 |

mRNA for P-450 IIA4 protein

| | | |
|---|---|---|
| 488 | 479A>T | H160L |

| | | | | |
|---|---|---|---|---|
| X14583 | X14583 | 147240 | GEN-1RJ | Human mRNA for Ig |

lambda-chain

| | | |
|---|---|---|
| 611 | 587C>T | A196V |

| | | | | |
|---|---|---|---|---|
| X52079 | X52079 | 602272 | GEN-33B | H.sapiens transcription |

factor (ITF-2) mRNA, 3′ end

| | | |
|---|---|---|
| 1794 | 1794G>A | Silent |

| | | | | |
|---|---|---|---|---|
| X52425 | X52425 | 147781 | GEN-59 | Interleukin 4 receptor |

| | | |
|---|---|---|
| 1902 | 1727A>G | Q576R |
| 3289 | 3114A>G | 3′ |
| 3391 | 3216C>T | 3′ |

| | | | | |
|---|---|---|---|---|
| X54199 | X54199 | 138440 | GEN-LS | Phosphoribosylglycin- |

amide formyltransferase, phosphoribosylglycinamide synthetase,
phosphoribosylaminoimidazole synthetase

| | | |
|---|---|---|
| 1339 | 1261G>A | V421I |
| 2333 | 2255A>G | D752G |

| | | | | |
|---|---|---|---|---|
| X57522 | X57522 | 170260 | GEN-37W | H.sapiens RING4 |

cDNA

| | | |
|---|---|---|
| 1207 | 1177A>G | I393V |

| | | | | |
|---|---|---|---|---|
| X57829 | X57829 | 112203 | GEN-4EW | Serotonin receptor |

5HT-1A, coding sequence except for stop codon

| | | |
|---|---|---|
| 47 | 47C>T | P16L |
| 64 | 64G>A | G22S |
| 82 | 82A>G | I28V |
| 294 | 294G>A | Silent |
| 551 | 551C>T | P184L |
| 659 | 659G>T | R220L |
| 818 | 818G>A | G273D |

| | | | | |
|---|---|---|---|---|
| X57830 | X57830 | 182135 | GEN-7V | Serotonin receptor |

5HT-2A, cDNA

| | | |
|---|---|---|
| 247 | 102T>C | Silent |
| 661 | 516C>T | Silent |
| 734 | 589A>G | I197V |
| 1485 | 1340C>T | A447V |
| 1499 | 1354C>T | H452Y |
| 1681 | 1536G>C | 3′ |

| | | | | |
|---|---|---|---|---|
| X60592 | X60592 | 109535 | GEN-3B0 | Human CDw40 |

mRNA for nerve growth factor receptor-related B-lymphocyte activation
molecule

| | | |
|---|---|---|
| 437 | 390G>T | Silent |

| | | | | |
|---|---|---|---|---|
| X62572 | X62572 | 146790 | GEN-3CL | H.sapiens RNA for Fc |

receptor, PC23

| | | |
|---|---|---|
| 487 | 487G>A | 3′ |
| 1240 | 1240A>G | 3′ |

| | | | | |
|---|---|---|---|---|
| X63359 | X63359 | 600070 | GEN-3DC | H.sapiens UGT2BIO |

mRNA for udp glucuronosyltransferase

| | | |
|---|---|---|
| 2714 | 2704G>A | 3′ |

| | | | | |
|---|---|---|---|---|
| X70697 | X70697 | 182138 | GEN-4DI | H.sapiens mRNA for |

serotonin transporter

| | | |
|---|---|---|
| 277 | 167G>C | G56A |

| | | | | |
|---|---|---|---|---|
| X75535 | X75535 | 600279 | GEN-3O8 | H.sapiens mRNA for |

PxF protein

| | | |
|---|---|---|
| 1808 | 1798A>G | 3′ |

| | | | | |
|---|---|---|---|---|
| X79389 | X79389 | 600436 | GEN-3T7 | H.sapiens GSTT1 |

mRNA

| | | |
|---|---|---|
| 824 | 824T>C | 3′ |

| | | | | |
|---|---|---|---|---|
| X83861 | X83861 | 176806 | GEN-5H | Prostaglandin E |

receptor 3 (subtype EP3) {alternative products}

| | | |
|---|---|---|
| 179 | (−29)C>T | 5′ |
| 318 | 111C>G | Silent |
| 712 | 505A>T | M169L |
| 825 | 618G>T | Silent |
| 1518 | 1311T>C | 3′ |
| 1593 | 1386A>G | 3′ |

| | | | | |
|---|---|---|---|---|
| X92106 | X92106 | 602403 | GEN-47S | H.sapiens mRNA for |

bleomycin hydrolase

| | | |
|---|---|---|
| 1405 | 1327A>G | I443V |

| | | | | |
|---|---|---|---|---|
| XDH | U06117 | 278300 | GEN-194 | Human xanthine |

dehydrogenase (XDH) mRNA, complete cds

| | | |
|---|---|---|
| 745 | 682C>T | Frame |
| 2630 | 2567delC | Frame |

| | | | | |
|---|---|---|---|---|
| Y10387 | Y10387 | None | GEN-1IU | H.sapiens mRNA for |

PAPS synthetase

| | | |
|---|---|---|
| 1981 | 1945G>A | 3′ |

| | | | | |
|---|---|---|---|---|
| Z30643 | Z30643 | 602024 | GEN-MMQ | H.sapiens mRNA for |

chloride channel (putative) 2139bp

| | | |
|---|---|---|
| 1711 | 1689G>A | Silent |

What is claimed is:

1. A method for selecting a treatment for a patient suffering from a disease disorder or condition, comprising
determining whether cells of said patient contain at least one variance in a gene from Tables 1, 3 and 4, wherein the presence or the absence of said variance in said gene is indicative of the effectiveness or safety of said treatment for said disease, disorder, or condition.

2. The method of claim 1, wherein said disease, disorder, or condition is selected from the group consisting of drug-induced diseases, disorders, or toxicities consisting of blood dyscrasias, cutaneous toxicities, systemic toxicities, central nervous system toxicities, hepatic toxicities, cardiovascular toxicities, pulmonary toxicities, and renal toxicities.

3. The method of claim 1, wherein the presence of said at least one variance is indicative that said treatment will be effective for said patient.

4. The method of claim 1, wherein the presence of said variance is indicative that said treatment will be ineffective or contra-indicated for said patient.

5. The method of claim 1, wherein said at least one variance comprises a plurality of variances.

6. The method of claim 5, wherein said plurality of variances comprise a haplotype or haplotypes.

7. The method of claim 1, wherein said selecting a treatment further comprises identifying a compound differentially active in a patient bearing a form of said gene containing said at least one variance.

8. The method of claim 7, wherein said compound is a compound listed in a Table herein or that belongs to the same chemical class as a compound listed in said Table.

9. The method of claim 1, wherein said selecting a treatment further comprises
eliminating or excluding a treatment, wherein said presence or absence of said at least one variance is indicative that said treatment will be ineffective or contra-indicated.

10. The method of claim 1, wherein said treatment comprises a first treatment and a second treatment, said method comprising the steps of:
identifying a said first treatment effective to treat said disease, disorder, or condition; and
identifying a said second treatment which reduces a deleterious effect or promotes efficacy of said first treatment.

11. The method of claim 1, wherein said selecting a treatment further comprises selecting a method of administration of a compound effective to treat said disease, disorder or condition, wherein said presence or absence of said at least one variance is indicative of the appropriate method of administration for said compound.

12. The method of claim 11, wherein said selecting a method of administration comprises selecting a suitable dosage level or frequency of administration of a compound.

13. The method of claim 1, further comprising determining the level of expression of said gene or the level of activity of a protein containing a polypeptide expressed from said gene,
wherein the combination of the determination of the presence or absence of said at least one variance and the determination of the level of activity or the level of expression provides a further indication of the effectiveness of said treatment.

14. The method of claim 1, further comprising determining the at least one of sex, age, racial origin, ethnic origin, and geopraphic origin of said patient,
wherein the combination of the determination of the presence or absence of said at least one variance and the determination of the sex, age, racial origin, ethnic origin, and geopraphic origin of said patient provides a further indication of the effectiveness of said treatment.

15. The method of claim 1, wherein said disease, disorder, or condition is selected from the group consisting of drug-induced diseases, disorders, or toxicities consisting of blood dyscrasias, cutaneous toxicities, systemic toxicities, central nervous system toxicities, hepatic toxicities, cardiovascular toxicities, pulmonary toxicities, and renal toxicities.

16. The method of claim 1, wherein the detection of the presence or absence of said at least one variance comprises amplifying a segment of nucleic acid including at least one of said variances.

17. The method of claim 16, wherein said segment of nucleic acid is 500 nucleotides or less in length.

18. The method of claim 16, wherein said segment of nucleic acid is 100 nucleotides or less in length.

19. The method of claim 16, wherein said segment of nucleic acid is 45 nucleotides or less in length.

20. The method of claim 16, wherein said segment includes a plurality of variances.

21. The method of claim 17, wherein amplification preferentially occurs from one of the two strands of a chromosome.

22. The method of claim 17, wherein said segment of nucleic acid is at least 500 nucleotides in length.

23. The method of claim 1, wherein the detection of the presence or absence of said at least one variance comprises contacting nucleic acid comprising a variance site with at least one nucleic acid probe, wherein said at least one probe preferentially hybridizes with a nucleic acid sequence including said variance site and containing a complementary base at said variance site under selective hybridization conditions.

24. The method of claim 1, wherein the detection of the presence or absence of said at least one variance comprises sequencing at least one nucleic acid sequence.

25. The method of claim 1, wherein the detection of the presence or absence of said at least one variance comprises mass spectrometric determination of at least one nucleic acid sequence.

26. The method of claim 1, wherein the detection of the presence or absence of said at least one variance comprises determining the haplotype of a plurality of variances in a gene.

27. A method for selecting a method of treatment, comprising
comparing at least one variance in at least one gene from Tables 1, 3 and 4 in a patient suffering from a disease or condition with a list of variances in said at least one gene indicative of the effectiveness of at least one method of treatment.

28. The method of claim 27, wherein said list comprises at least 5 variances.

29. The method of claim 27, wherein said at least one variance comprises a plurality of variances.

30. The method of claim 27, wherein said list of variances comprises a plurality of variances.

31. The method of claim 27, wherein at least one said method of treatment comprises the administration of a compound effective against said disease or condition to a patient.

32. The method of claim 31, wherein said compound is selected from the group consisting of agonists, antagonists, blockers, partial agonists, partial antagonists, inhibitors, activators, modulators, negative antagonists, inverse agonists, mimetics, or factors that elicit pharmacological activity on a gene product of at least one gene or gene pathway listed in Table 1.

33. The method of claim 27, wherein the presence or absence of at least one variance or haplotype in said gene is indicative that said treatment will be effective in said patient.

34. The method of claim 27, wherein the presence or absence of at least one variance in said gene is indicative that said treatment will be ineffective or contra-indicated.

35. The method of claim 27, wherein said treatment is a first treatment and the presence or absence of at least one variance in said gene is indicative that a second treatment will be beneficial to reduce a deleterious effect or promotes efficacy of said first treatment.

36. The method of claim 27, wherein said at least one method of treatment is a plurality of methods of treatment.

37. The method of claim 36, wherein said selecting comprises determining whether any of said plurality of methods of treatment will be more effective than at least one other of said plurality of methods of treatment.

38. The method of claim 27, wherein said disease is from the group consisting of drug-induced diseases, disorders, or toxicities consisting of blood dyscrasias, cutaneous toxicities, systemic toxicities, central nervous system toxicities, hepatic toxicities, cardiovascular toxicities, pulmonary toxicities, and renal toxicities.

39. A method for selecting a method of administration of to a patient suffering from a condition or disease for a compound or compounds effective to treat said condition or disease, comprising
determining whether at least one variance in a gene from Tables 1, 3 and 4 is present or absent in cells of said patient, wherein said presence or absence of said at least one variance is indicative of an appropriate method of administration for said compound.

40. The method of claim 39, wherein said at least one variance is a plurality of variances.

41. The method of claim 39, wherein said selecting a method of administration comprises selecting a dosage level or frequency or frequency of administration of said compound.

42. The method of claim 39, wherein said compound is selected from the group consisting of agonists, antagonists, blockers, partial agonists, partial antagonists, inhibitors, activators, modulators, negative antagonists, inverse agonists, mimetics, or factors that elicit pharmacological activity on a gene product of at least one gene or gene pathway listed in Table 1.

43. The method of claim 39, wherein said disease is from the group consisting of drug-induced diseases, disorders, or toxicities consisting of blood dyscrasias, cutaneous toxicities, systemic toxicities, central nervous system toxicities, hepatic toxicities, cardiovascular toxicities, pulmonary toxicities, and renal toxicities.

44. A method for selecting a patient for administration of a method of treatment, comprising
comparing the presence or absence of at least one variance or haplotype in a gene from Tables 1, 3 and 4 in cells of a patient suffering from a disease or condition with a list of variances in said at least one gene, wherein the presence or absence of said at least one variance or haplotype in said cells is indicative that said treatment will be effective, more effective, less effective, ineffective, or contra-indicated in said patient; and
determining whether said patient will receive said method of treatment based on the presence or absence of said at least one variance in said cells.

45. The method of claim 44, wherein said list comprises at least 5 variances.

46. The method of claim 44, wherein said method of treatment comprises administration of a compound effective against said disease or condition.

47. The method of claim 46, wherein said compound is selected from the group consisting of agonists, antagonists, blockers, partial agonists, partial antagonists, inhibitors, activators, modulators, negative antagonists, inverse agonists, mimetics, or factors that elicit pharmacological activity on a gene product of at least one gene or gene pathway listed in Table 1.

48. A method for identifying the presence or absence of at least one form of a gene from Tables 1, 3 and 4 in cells of an individual, comprising:
determining the presence or absence of at least one variance in said gene in said cells.

49. The method of claim 48, wherein said said at least one variance is a plurality of variances.

50. The method of claim 49, wherein said plurality of variances comprises a haplotype.

51. The method of claim 50, wherein said individual suffers from a disease or condition.

52. The method of claim 50, wherein the presence or absence of said at least one variance is indicative of the effectiveness of a therapeutic treatment in a patient having cells containing said at least one variance.

53. The method of claim 50, wherein said determining comprises amplifying a segment of nucleic acid including a site of at least one variance.

54. The method of claim 50, wherein said determining comprises contacting a nucleic acid sequence containing a variance site corresponding to a said variance with a probe which specifically binds under selective binding conditions to a nucleic acid sequence comprising at least one said variance.

55. The method of claim 50, wherein the detection of the presence or absence of said at least one variance comprises sequencing at least one nucleic acid sequence.

56. The method of claim 50, wherein the detection of the presence or absence of said at least one variance comprises mass spectrometric determination of at least one nucleic acid sequence.

57. The method of claim 50, wherein the detection of the presence or absence of said at least one variance comprises determining the haplotype of a plurality of variances in a gene.

58. A pharmaceutical composition comprising
a compound which has a differential effect in patients having at least one copy of a particular form of an identified gene from Tables 1, 3 and 4; and
a pharmaceutically acceptable carrier or excipient or diluent,
wherein said composition is preferentially effective to treat a patient with cells comprising a form of said gene comprising at least one variance.

59. The method of claim 58, wherein said composition is adapted to be preferentially effective based on the unit dosage, presence of additional active components, complexing of said compound with stabilizing components, or inclusion of components enhancing delivery or slowing excretion of said compound.

60. The composition of claim 58, wherein said compound is deleterious to patients having said at least one copy or in patients not having said at least one copy, but not in both.

61. The composition of claim 58, wherein said patient suffers from a disease or condition selected from the group consisting of drug-induced diseases, disorders, or toxicities consisting of blood dyscrasias, cutaneous toxicities, systemic toxicities, central nervous system toxicities, hepatic toxicities, cardiovascular toxicities, pulmonary toxicities, and renal toxicities.

62. The pharmaceutical composition of claim 58, wherein said composition is subject to a regulatory restriction or recommendation for use of a diagnostic test determining the presence or absence of at least one variance or haplotype in said gene.

63. The pharmaceutical composition of claim 58, wherein said pharmaceutical composition is subject to a regulatory limitation or recommendation restricting or recommending restriction of the use of said pharmaceutical composition to patients having at least one copy of a form of a gene comprising at least one variance.

64. The pharmaceutical composition of claim 58, wherein said pharmaceutical composition is subject to a regulatory limitation or recommendation indicating said pharmaceutical composition is not to be used in patients having at least one copy of a form of a gene comprising at least one variance.

65. The pharmaceutical composition of claim 58, wherein said pharmaceutical composition is packaged, and the packaging includes a label or insert restricting or recommending the restriction of the use of said pharmaceutical composition to patients having at least one copy of a form of a gene comprising at least one variance or haplotype.

66. The pharmaceutical composition of claim 58, wherein said pharmaceutical composition is packaged, and said packaging includes a label or insert requiring or recommending the use of a test to determine the presence or absence of at least one variance in cells of a said patient.

67. A nucleic acid probe comprising a nucleic acid sequence 7 to 200 nucleotide bases in length that specifically binds under selective binding conditions to a nucleic acid sequence comprising at least one variance in a gene from Tables 1, 3 and 4, or a sequence complementary thereto or an RNA equivalent.

68. The probe of claim 67, wherein said probe comprises a nucleic acid sequence 500 nucleotide bases or fewer in length.

69. The probe of claim 67, wherein said nucleic acid sequence is 100 or fewer nucleotide bases in length.

70. The probe of claim 67, wherein said nucleic acid sequence is 25 or fewer nucleotide bases in length.

71. The probe of claim 67, wherein said probe comprises DNA.

72. The probe of claim 67, wherein said probe comprises DNA and at least one nucleic acid analog.

73. The probe of claim 67, wherein said probe comprises peptide nucleic acid (PNA).

74. The probe of claim 67, further comprising a detectable label.

75. The probe of claim 74, wherein said detectable label is a fluorescent label.

76. A method for determining a genotype of an individual, comprising analyzing at least one nucleic acid sequence from cells of said individual using mass spectrometric analysis,
wherein said nucleic acid sequence is a portion of a gene from Tables 1, 3 and 4 or a sequence complementary thereto.

77. The method of claim 76, wherein said analyzing a nucleic acid sequence comprises determining the presence or absence of a variance in said gene.

78. The method of claim 76, wherein said analyzing a nucleic acid sequence comprises determining the nucleotide sequence of said at least one nucleic acid sequence.

79. The method of claim 76, wherein said at least one nucleic acid sequence is 500 nucleotides or less in length.

80. The method of claim 76, wherein said at least one nucleic acid sequence comprises at least one variance site in said gene.

81. An isolated, purified or enriched nucleic acid sequence of 15 to 500 nucleotides in length, comprising at least one variance site, wherein said sequence has the base sequence of a portion of an allele of a gene from Tables 1, 3 and 4.

82. The nucleic acid sequence of claim 81, wherein said nucleic acid sequence is 15 to 100 nucleotide bases in length.

83. The nucleic acid sequence of claim 81, wherein said nucleic acid sequence is 15 to 25 nucleotide bases in length.

84. A method for determining whether a compound has differential effects on cells containing at least one different form of a gene from Tables 1, 3 and 4, comprising:
contacting a first cell and a second cell with said compound, wherein said first cell and said second cell differ in the presence or absence of at least one variance in said gene; and
determining whether the responses of said first cell and said second cell to said compound differ, wherein the difference in said response is due to the presence or absence of said at least one variance.

85. The method of claim 84, wherein said at least one variance comprises a haplotype.

86. The method of claim 84, wherein at least one of said first cell and said second cell are contacted in vivo.

87. The method of claim 85, wherein at least one of said first cell and said second cell are contacted in vitro.

88. The method of claim 87, wherein at least one of said first cell and said second cell is contacted in vivo in a plurality of patients suffering from a disease or condition.

89. A method of treating a patient suffering from a condition or disease, comprising:
a) determining whether cells of said patient contain a form of a gene from Tables 1, 3 and 4 which comprises at least one variance, wherein the presence or absence of said at least one variance is indicative that a treatment will be effective in said patient; and
b) administering said treatment to said patient.

90. The method of claim 89, wherein said gene is listed in Table 1 or is a gene in a pathway listed in Table 1 herein.

91. The method of claim 89, wherein said disease or condition is selected from the group consisting of drug-induced diseases, disorders, or toxicities consisting of blood dyscrasias, cutaneous toxicities, systemic toxicities, central nervous system toxicities, hepatic toxicities, cardiovascular toxicities, pulmonary toxicities, and renal toxicities.

92. The method of claim 89, wherein said at least one variance is a plurality of variances.

93. The method of claim 89, wherein the presence of said at least one variance is indicative that said treatment will be effective in said patient.

94. The method of claim 93, wherein said treatment comprises the administration of a compound preferentially active for said condition or disease in a said patient having said at least one variance in said gene.

95. The method of claim 94, wherein said compound is selected from the group consisting of agonists, antagonists, blockers, partial agonists, partial antagonists, inhibitors, activators, modulators, negative antagonists, inverse agonists, mimetics, or factors that elicit pharmacological activity on a gene product of at least one gene or gene pathway listed in Table 1.

96. The method of claim 89, wherein the presence of said at least one variance in said gene is indicative of an appropriate dosage or frequency of administration of a compound in said treatment.

97. A method of treating a patient suffering from a disease or condition, comprising:
a) comparing the presence or absence of at least one variance in a gene from Tables 1, 3 and 4 in cells of a patient suffering from said disease or condition with a list of variances in said gene indicative of the effectiveness of at least one method of treatment;
b) selecting a method of treatment from said at least one method of treatment, wherein the presence or absence of at least one of said at least one variance is indicative that said method of treatment will be effective in said patient; and
c) administering said method of treatment to said patient.

98. The method of claim 97, wherein said at least one gene comprises a gene listed in Table 1 or comprises a gene in a pathway listed in Table 1 herein.

99. The method of claim 97, wherein said condition or disease is a condition or disease listed in the Detailed Description, Examples, or Tables herein.

100. The method of claim 97, further comprising determining the presence or absence of said at least one variance in cells of said patient.

101. The method of claim 97, wherein said at least one variance comprises a plurality of variances.

102. The method of claim 97, wherein said list of variances comprises a plurality of variances.

103. The method of claim 102, wherein said plurality of variances comprises a haplotype or haplotypes.

104. The method of claim 97, wherein said method of treatment comprises the administration of a compound effective against said disease or condition.

105. The method of claim 97, wherein said treatment is a first treatment and the presence or absence of at least one variance in said gene is indicative that a second treatment will be beneficial to reduce a deleterious effect or promotes efficacy of said first treatment.

106. The method of claim 97, wherein said at least one method of treatment is a plurality of methods of treatment.

107. The method of claim 97, wherein said disease or condition is selected from the group consisting of drug-induced diseases, disorders, or toxicities consisting of blood dyscrasias, cutaneous toxicities, systemic toxicities, central nervous system toxicities, hepatic toxicities, cardiovascular toxicities, pulmonary toxicities, and renal toxicities.

108. A method of treating a patient suffering from a disease or condition, comprising:
a) comparing the presence or absence of at least one variance in a gene from Tables 1, 3 and 4 in cells of a patient suffering from said disease or condition with a list of variances in said gene indicative of the effectiveness of at least one method of treatment;
b) eliminating or excluding a method of treatment from said at least one method of treatment, wherein the presence or absence of at least one of said at least one variance is indicative that said method of treatment will be ineffective or contra-indicated in said patient;
c) selecting an alternative method of treatment effective to treat said cardiovascular or renal disease or condition; and
d) administering said alternative method of treatment to said patient.

109. The method of claim 108, further comprising determining the presence or absence of said at least one variance in cells of said patient.

110. The method of claim 108, wherein said gene is listed in Table 1 or is a gene in a pathway listed in Table 1 herein.

111. The method of claim 108, wherein said disease or condition is a disease or condition listed in the Detailed Description, Examples, or Tables herein.

112. A method for producing a pharmaceutical composition, comprising:
a) identifying a compound which has differential activity against a disease or condition in patients having at least one variance in a gene from Tables 1, 3 and 4;
b) compounding said pharmaceutical composition by combining said compound and a pharmaceutically acceptable carrier or excipient or diluent in a manner adapted to be preferentially effective in patients having said at least one variance.

113. A method for producing a pharmaceutical agent, comprising:
a) identifying a compound which has differential activity against a disease or condition in patients having at least one variance in a gene from Tables 1, 3 and 4; and
b) synthesizing said compound in an amount sufficient to provide a pharmaceutical effect in a patient suffering from said cardiovascular or renal disease or condition.

114. A method for determining whether a variance in a gene from Tables 1, 3 and 4 provides variable patient response to a method of treatment for a disease or condition, comprising:
determining whether the response of a first patient or set of patients suffering from said disease or condition differs from the response of a second patient or set of patients suffering from said disease or condition; and
determining whether the presence or absence of at least one variance in said gene differs between said first patient or set of patient and said second patient or set of patients,
wherein correlation of said presence or absence of at least one variance and the response of said patient to said treatment is indicative that said at least one variance provides variable patient response.

115. The method of claim 114, further comprising identifying at least one variance in a said gene.

116. The method of claim 114, wherein a plurality of pairwise comparisons of treatment response and the presence or absence of at least one variance are performed for a plurality of patients.

117. The method of claim 114, wherein said determining whether the presence or absence of at least one variance in at least one gene comprises comparing the response of at least one patient homozygous for said at least one variance with at least one patient homozygous for the alternative form of said at least one variance.

118. The method of claim 114, wherein said determining whether the presence or absence of said at least one variance in at least one gene comprises comparing the response of at least one patient heterozygous for said at least one variance with the response of at least one patient homozygous for said at least one variance.

119. The method of claim 114, wherein it is previously known that patient response to said method of treatment is variable.

120. The method of claim 114, wherein said disease or condition is a disease or condition listed in the Detailed Description, Examples, or Tables herein.

121. The method of claim 114, wherein said disease or condition is selected from the group consisting of drug-induced diseases, disorders, or toxicities consisting of blood dyscrasias, cutaneous toxicities, systemic toxicities, central nervous system toxicities, hepatic toxicities, cardiovascular toxicities, pulmonary toxicities, and renal toxicities.

122. The method of claim 114, wherein said method of treatment comprises administration of a compound effective to treat said disease or condition.

123. A method of treating a disease, condition, or a drug-induced disease in a patient, comprising
a) selecting a patient whose cells comprise an allele of a gene from Tables 1, 3 and 4, wherein said allele comprises at least one variance correlated with more effective treatment of said disease or condition; and
b) altering the level of activity in cells of said patient of a product of said allele, wherein said altering provides a therapeutic effect.

124. A method for determining a method of treatment effective to treat a disease or condition in a sub-population of patients, comprising
altering the level of activity of a product of an allele of a gene from Tables 1, 3 and 4; and
determining whether said alteration provides a differential effect related to reducing or alleviating a disease or condition as compared to at least one alternative allele, wherein the presence of a said differential effect is indicative that said altering the level of activity comprises an effective treatment for said disease or condition in said sub-population.

125. A method for performing a clinical trial or study, comprising
selecting or stratifying subjects using a variance or variances or haplotypes from one or more genes specified in Tables 1, 3 or 4.

126. The method of claim 125, wherein differential efficacy, tolerance, or safety of a treatment in a subset of patients who have a particular variance, variances, or haplotype in a gene or genes from Tables 1, 3 and 4, comprising conducting a clinical trial and using a statistical test to assess whether a relationship exists between efficacy, tolerance, or safety with the presence or absence of any of said variances or haplotype in one or more of said genes,
wherein results of said clinical trial or study are indicative whether a higher or lower efficacy, tolerance, or safety of said treatment in said subset of patients is associated with any of said variance or variances or haplotype in one or more of said gene.

127. The method of claim 125 wherein normal subjects or patients are prospectively stratified by genotype in different genotype-defined groups, including the use of genotype as a enrollment criterion, using a variance, variances or haplotypes from Tables 1, 3 or 4, and subsequently a biological or clinical response variable is compared between the different genotype-defined groups.

128. The method of claim 125 wherein the normal subjects or patients in a clinical trial or study are stratified by a biological or clinical response variable in different biologically or clinically-defined groups, and subsequently the frequency of a variance, variances or haplotypes from Tables 1, 3 or 4 is measured in the different biologically or clinically defined groups.

129. The method of claim 127 or 128 where the normal subjects or patients in a clinical trial or study are stratified by at least one demographic characteristic selected from the groups consisting of sex, age, racial origin, ethnic origin, or geographic origin.

130. The method of claim 125, wherein said determining comprises assigning said patient to a group to receive said method of treatment or to a control group.

131. A method for determining whether a variance in a gene provides variable patient response to a method of treatment for a disease or condition, comprising:
determining whether the response of a first patient or set of patients suffering from a disease, condition, or drug-induced disease differs from the response of a second patient or set of patients suffering from said disease or condition;
determining whether the presence or absence of at least one variance in a gene from Tables 1, 3 and 4 differs between said first patient or set of patients and said second patient or set of patients;
wherein correlation of said presence or absence of at least one variance and the response of said patient to said treatment is indicative that said at least one variance provides variable patient response.

132. A method for treating a patient at risk for a disease or diagnosed with a disease or disorder or a drug induced disease, comprising
identifying a said patient and determining the patient's genotype allele status for a gene from Tables 1, 3 and 4;
determining a treatment protocol using the patient's genotype status to provide a prediction of the efficacy and safety of a therapy in light of said disease or an associated condition.

133. A method for identifying a patient for participation in a clinical trial of a therapy for the treatment of a disease or a drug-associated disease or disorder, comprising
identifying a patient with a disease risk and determining the patient's genotype, allele status for an identified gene from Tables 1, 3 and 4.

134. The method of claim 123, further comprising
determining the patient's allele status and selecting those patients having at least one wild type allele of said gene as candidates likely to be affected by a drug-induced disease or condition.

135. A method for treating a patient at risk for a disease condition, comprising
identifying a patient with a risk for said disease;
determining the genotypic allele status of the patient for at least one gene from Tables 1, 3 and 4; and
converting the genotypic allele status into a treatment protocol that comprises a comparison of the genotypic allele status determination with the allele frequency of a control population, thereby allowing a statistical calculation of the patient's risk for having said disease or condition.

136. A method for treating a patient at risk for or diagnosed with having a disease or condition, comprising
identifying a said patient;
determining the gene allele load status of the patient for at least one gene from Tables 1, 3 and 4 and converting the gene allele load status into a treatment protocol that includes a comparison of the allele status determinations with the allele frequency of a control population, thereby allowing a statistical calculation of the patient's risk for having having said disease or condition.

137. A method for improving the safety of candidate therapies associated with having a disease or condition, comprising
comparing the relative safety of the candidate therapeutic intervention in patients having different alleles in one or more than one of the genes listed in Tables 1, 3 and 4, thereby identifying subsets of patients with differing safety of the candidate therapeutic intervention.

138. A kit for determination of the presence or absence of at least one sequence variance in a gene identified in any of Tables 1, 3, and 4, comprising
at least one probe that preferentially hybridizes with a nucleic acid sequence corresponding to a portion of said gene or at least one primer comprising a nucleic acid sequence corresponding to a portion of said gene or a sequence complementary thereto or both said at least one probe and said at least one primer.

139. A method for determining whether there is a genetic component to intersubject variation in a surrogate treatment response, comprising:
a. administering said treatment to a group of related normal subjects and a group of unrelated normal subjects;
b. measuring a surrogate pharmacodynamic or pharmacokinetic drug response variable in said subjects;
c. performing a statistical test measuring the variation in response in said group of related normal subjects and, separately in said group or unrelated normal subjects; and
d. comparing the magnitude or pattern of variation in response or both between said groups to determine if the responses of said groups are different, using a predetermined statistical measure of difference,
wherein a difference in response between said groups is indicative that there is a genetic component to intersubject variation in said surrogate treatment response.

140. The method of claim 129, wherein the size of the related and unrelated groups is set in order to achieve a predetermined degree of statistical power.

141. A method for evaluating the combined contribution of two or more variances to a surrogate drug response phenotype in subjects, comprising:
a. genotyping a set of unrelated subjects participating in a clinical trial or study of a compound for two or more variances to identify subjects with specific genotypes, wherein said two or more specific genotypes define two or more genotype-defined groups;
b. administering a drug to subjects with two or more of said specific genotypes;
c. measuring a surrogate pharmacodynamic or pharmacokinetic drug response variable in said subjects;
d. performing statistical tests to measure response in said groups separately, wherein said statistical tests provide a measurement of variation in response with each said group; and
e. comparing the magnitude or pattern of variation in response or both between said groups to determine if said groups are different using a predetermined statistical measure of difference.

142. The method of claim 141, wherein said clinical trial or study is a Phase I clinical trial or study.

143. The method of claim 141, wherein said specific genotypes are homozygous genotypes for two variances.

144. The method of claim 141, wherein the comparison is between groups of subjects differing in three or more variances.

145. A method for providing contract research services to a client, comprising:
a. enrolling subjects in a clinical drug trial or study unit for the purpose of genotyping said subjects in order to assess the contribution of one or more variances or haplotypes to variation in drug response;
b. genotyping said subjects to determine the status of one or more variances in said subjects;
c. administering a compound to said subjects and measuring a surrogate drug response variable;
d. comparing responses between two or more genotype-defined groups of said subjects to determine whether there is a genetic component to the interperson variability in response to said compound; and
e. reporting the results of said clinical drug trial or study unit to a contracting entity.

146. The method of claim 145, wherein said clinical drug trial or study unit is a Phase I drug trial or study unit.

147. The method of claim 145, wherein at least some of the subjects have disclosed that they are related to each other and said comparing includes comparison of groups of related individuals.

148. The method of claim 147, wherein the related individuals are encouraged to participate by compensation in proportion to the number of their relatives participating.

149. A method for recruiting a clinical trial or study population for studies of the influence of one or more variances or haplotypes on drug response, comprising
soliciting subjects to participate in said clinical trial or study;
obtaining consent of said subjects for participation in said clinical trial or study; and
obtaining additional related subjects for participation in said clinical trial by compensating one or more of the related subjects for said participation at a level based on the number of related subjects participating or based on participation of at least a minimum specified number of related subjects.
